(19) United States
(12) Patent Application Publication
(10) Pub. No.: US 2025/0263408 A1
(43) Pub. Date: Aug. 21, 2025

(54) SPLEEN TYROSINE KINASE INHIBITORS

(71) Applicant: Uniquest Pty Ltd, St. Lucia (AU)

(72) Inventors: Eun Jung Ko, St. Lucia, Queensland (AU); Andrew Harvey, St. Lucia, Queensland (AU); Brian Dymock, St. Lucia, Queensland (AU); Jacek Mikolaj Kwiatkowski, St. Lucia, Queensland (AU); David Michael Pinkerton, St. Lucia, Queensland (AU); Therese Eliza Johnson, St. Lucia, Queensland (AU); Nicholas John Matovic, St. Lucia, Queensland (AU); Malika Dhananjaya Kumarasiri, St. Lucia, Queensland (AU); Richard Gordon, St. Lucia, Queensland (AU)

(21) Appl. No.: 18/854,221

(22) PCT Filed: Apr. 6, 2023
(86) PCT No.: PCT/AU2023/050275

§ 371 Date: Oct. 4, 2024

(30) Foreign Application Priority Data
Apr. 7, 2022 (AU) 2022900917

(51) Int. Cl. C07D 471/04; C07D 413/14; C07D 519/00

(52) U.S. Cl. CPC C07D 471/04; C07D 413/14; C07D 519/00

(57) ABSTRACT

The present invention relates to compounds of formula (I), or a pharmaceutically acceptable salt or prodrug thereof:

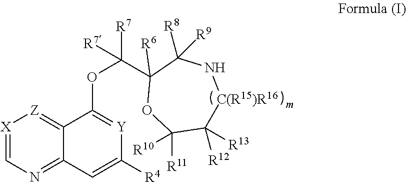

in which R⁴ is 5-membered cycloalkene or 5-membered heteroaryl, each of which is optionally fused to form a 5:6, or 5:5 aromatic or heteroaromatic bicycle; wherein each R⁴ is optionally substituted. The present invention also relates to a pharmaceutical composition comprising the compounds, and to uses of the compounds, especially in the treatment of a disease, disorder or condition associated with spleen tyrosine kinase activity.

### TECHNICAL FIELD

The present invention relates, inter alica, to compounds, pharmaceutical compositions of said compounds, and uses of said compounds. The compounds are especially for inhibition of spleen tyrosine kinase.

### BACKGROUND ART

It will be clearly understood that, if a prior art publication is referred to herein, this reference does not constitute an admission that the publication forms part of the common general knowledge in the art in Australia or in any other country.

Spleen tyrosine kinase (Syk) is a cytoplasmic non-receptor kinase that plays a central role in mediating inflammatory responses. Upon activation by membrane-bound receptors, Syk phosphorylates numerous downstream targets which are primarily responsible for the development and function of immune cells including B-cells, T-cells, dendritic cells, Natural killer (NK) cells, mast cells, basophils, macrophages and microglia (Turner et al. 2000; Sedlik et al. 2003; Yi et al. 2014; Lee and Suk 2018).

Syk is known to be upregulated and plays an important role in neuroinflammatory diseases, autoimmune diseases, allergies and B-cell malignancies. Syk is a potential target for the treatment of glioblastoma (Moncayo et al. 2018), ovarian cancer (Yu et al. 2019), 13-cell and T-cell lymphomas (Geahlen 2014), Type I diabetes (Geahlen 2014), cutaneous and systemic lupus erythematosus (Braegelmann et al. 2016; Grammatikos et al. 2013; Wong et al. 2004), rheumatoid arthritis (Pine et al. 2007; Coffey et al. 2012; Wong et al. 2004), gout (Mócsai, Ruland, and Tybulewicz 2010), multiple sclerosis (Wong et al. 2004), type I hypersensitivity reactions including allergic rhinitis, urticaria, asthma and anaphylaxis asthma, and allergic rhinitis (Wong et al. 2004), different liver diseases including liver fibrosis, viral hepatitis, alcoholic liver disease, non-alcoholic steatohepatitis and hepatocellular carcinoma (Kurniawan et al. 2020; Bukong et al. 2016; Qu et al 2018), retinoblastoma (Zhang et al. 2012), peritoneal fibrosis (Liu et al. 2019), Lipopolysaccharide/Cigarette Smoke-Induced Airway Inflammation (Fan et al. 2019), head and neck cancer (Black et al. 2020), periodontal diseases (Kittaka et al. 2020), Graves' disease, hantavirus pulmonary syndrome, rapidly progressive glomerulonephritis, macroglobulinemia, epidermolysis bullosa acquisita, Wiskott-Aldrich syndrome, agammaglobulinemia, polycystic lipomembranous osteodysplasia with sclerosing leukoencephalopathy (Nasu-Hakola disease) (Mócsai, Ruland, and Tybulewicz 2010), co-trimoxazole allergy, fasciitis, Mycobacterium abscessus infection, autoimmune hypersensitivity disease, bleeding disorders, chromoblastomycosis, carbapenem allergy, Waldenstroem's macroglobulinemia (Munshi et al. 2020), thrombocytopenia (Bussel et al. 2018), Melnick-needles syndrome, nail disease, otopalatodigital syndrome spectrum disorders, abnormal bone metabolism (Shao et al. 2021), fungal infectious disease/mycosis, chronic mucocutaneous candidiasis and dermatitis (Pavel et al. 2019) and thrombotic cardiovascular diseases (Andre et al. 2011).

Syk inhibitors are currently marketed (Fostamnatinib) or being advanced in the clinic (Entospletinib) for peripheral indications, including inflammatory diseases and oncology, providing strong evidence for the suitability of Syk inhibitors as potential pharmaceuticals. Syk is highly conserved across species and current clinical compounds have displayed comparable in vitro activity against Syk orthologs from human, mouse and rat (Lamb et al. 2016; Currie et al. 2014), confirming the conservation of Syk structure and the ability to investigate using Multiple species.

Marketed and clinical Syk inhibitors (Fostamatinib and Entospletinib) support the safety profile of Syk inhibition. Fostamatinib has been tested extensively in long term studies for several disease indications, and adverse events were mild or manageable with dose reduction, interruption or secondary medication (Bussel et al. 2018; Kang et al. 2019). While Syk is expressed in a majority of cell types, including neurons, astrocytes and microglia (Flatterer et al. 2011; Xu et al. 2019; Lee and Suk 2018), high levels of expression are predominantly restricted to haematopoietic cells, including B-Cells, T-Cells, Mast Cells, Macrophages and Neutrophils. The human safety data is supported in mice with an inducible knockout of Syk, which displayed some reduced inflammatory responses but otherwise had no overt effects on basic body functions (Wex et al. 2011).

Clinical trials with antibodies directed at Syk-related pathways have also been studied further supporting the safety and efficacy of a direct Syk inhibitor. For example, TREM2 is a receptor that is associated with increased risk for Alzheimer's Disease (AD) and is also known to signal via Syk. Phase II trials with antibodies directed at TREM2 are being initiated. Phase 1 clinical trials of antibodies directed at two receptors (TREM2 and Siglec-3) in the same inflammatory pathway as Syk are ongoing (Alector, AL002 and AL003). An advantage of a direct Syk inhibitor is that multiple pathways are targeted, as opposed to one or two receptors. Coupled with the positive clinical safety profile of peripherally restricted Syk inhibitors, established with marketed drugs (Fostamatinib) and clinical inhibitors such as Entospletinib, direct Syk inhibition is highly desirable and may provide a significant advantage over other untested preclinical drug targets.

### SUMMARY OF INVENTION

With the foregoing in view, the present invention in one aspect is directed towards small molecules which inhibit Spleen Tyrosine Kinase (Syk).

In one aspect, the present invention is directed, inter alia, to compounds or a pharmaceutically acceptable salt or prodrug thereof which are Syk inhibitors.

In a first aspect, the present invention provides a compound of formula (I) or a pharmaceutically acceptable salt or prodrug thereof:

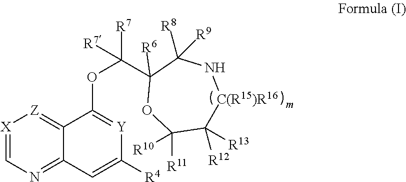

wherein:
Z is CR¹ or N;
Y is CH or N;
X is CR² or N; and
no more than one of X, Y or Z is N;
   wherein:
R¹ is selected from the group consisting of: hydrogen, C₁₋₆alkyl, C₁₋₆fluoroalkyl, C₂₋₆alkenyl, C₂₋₆fluoroalkenyl, C₂₋₆alkynyl, C₂₋₆fluoroalkynyl, C₃₋₆cycloalkyl, halo, —O—C₁₋₆alkyl, —O—C₁₋₆fluoroalkyl, —O—C₂₋₆alkenyl, —O—C₂₋₆fluoroalkenyl, —O—C₂₋₆alkynyl, —O—C₂₋₆fluoroalkynyl and cyano;
R² is selected from the group consisting of: hydrogen, C₁₋₆alkyl, C₁₋₆fluoroalkyl, C₂₋₆alkenyl, C₂₋₆fluoroalkenyl, C₂₋₆alkynyl, C₂₋₆fluoroalkynyl, halo, —O—C₁₋₆alkyl, —O—C₁₋₆fluoroalkyl, —O—C₂₋₆alkenyl, —O—C₂₋₆fluoroalkenyl, —O—C₂₋₆alkynyl, —O—C₂₋₆fluoroalkynyl and cyano;
R⁴ is 5-membered cycloalkene or 5-membered heteroaryl, each of which is optionally fused to form a 5:6, or 5:5 aromatic or heteroaromatic bicycle; wherein each R⁴ is optionally substituted;
n is 0 or 1;
R⁶ is selected from the group consisting of: H, C₁₋₆alkyl, C₁₋₆fluoroalkyl, C₃₋₆cycloalkyl, C₃₋₆fluorocycloalkyl, C₂₋₆alkenyl, C₂₋₆fluoroalkenyl, C₂₋₆alkynyl and C₂₋₆fluoroalkynyl;
R⁷ and R^{7′} are independently selected from the group consisting of: H, fluoro, C₁₋₆alkyl, C₁₋₆fluoroalkyl, C₃₋₆cycloalkyl, C₃₋₆fluorocycloalkyl, C₂₋₆alkenyl, C₂₋₆fluoroalkenyl, C₂₋₆alkynyl and C₂₋₆fluoroalkynyl; or R⁷ and R^{7′} together form a 3 to 6-membered cycloalkyl ring, a 3 to 6-membered fluorocycloalkyl ring, or a 4 to 6-membered oxygen containing heterocyclic ring;
R⁸ and R⁹ are independently selected from the group consisting of: H, C₁₋₆alkyl, C₁₋₆fluoroalkyl, C₃₋₆cycloalkyl, C₃₋₆fluorocycloalkyl, C₂₋₆alkenyl, C₂₋₆fluoroalkenyl, C₂₋₆alkynyl and C₂₋₆fluoroalkynyl; or R⁸ and R⁹ together form a 3 to 6-membered cycloalkyl ring, a 3 to 6-membered fluorocycloalkyl ring, or a 4 to 6-membered oxygen containing heterocyclic ring;
R¹⁰ and R¹¹ are independently selected from the group consisting of: H, C₁₋₆alkyl, fluoro, C₁₋₆fluoroalkyl, C₃₋₆cycloalkyl, C₃₋₆fluorocycloalkyl, C₂₋₆alkenyl, C₂₋₆fluoroalkenyl, C₂₋₆alkynyl and C₂₋₆fluoroalkynyl; or R¹⁰ and R¹¹ together form a 3 to 6-membered cycloalkyl ring, a 3 to 6-membered fluorocycloalkyl ring, or a 4 to 6-membered oxygen containing heterocyclic ring;
R¹² and R¹³ are independently selected from the group consisting of: H, C₁₋₆alkyl, fluoro, C₁₋₆fluoroalkyl, C₃₋₆cycloalkyl, C₃₋₆fluorocycloalkyl, C₂₋₆alkenyl, C₂₋₆fluoroalkenyl, C₂₋₆alkynyl and C₂₋₆fluoroalkynyl; or R¹² and R¹³ together form a 3 to 6-membered cycloalkyl ring, a 3 to 6-membered fluorocycloalkyl ring, or a 4 to 6-membered oxygen containing heterocyclic ring;
R¹⁵ and R¹⁶ are independently selected from the group consisting of: H, C₁₋₆alkyl, C₁₋₆fluoroalkyl, C₃₋₆cycloalkyl, C₃₋₆fluorocycloalkyl, C₂₋₆alkenyl, C₂₋₆fluoroalkenyl, C₂₋₆alkynyl and C₂₋₆fluoroalkynyl; or R¹⁵ and R¹⁶ together form a 3 to 6-membered cycloalkyl ring, a 3 to 6-membered fluorocycloalkyl ring, or a 4 to 6-membered oxygen containing heterocyclic ring; or wherein:
   one of R⁷ or R^{7′} and one of R⁸ or R⁹ together form a 5 or 6-membered cycloalkyl ring, a 5 or 6-membered fluorocycloalkyl ring, or a 5 or 6-membered oxygen containing heterocyclic ring;
   one of R⁷ or R^{7′} and R⁶ together form a 3 to 6-membered cycloalkyl ring, a 3 to 6-membered fluorocycloalkyl ring, or a 4 to 6-membered oxygen containing heterocyclic ring;
   R⁶ and one of R⁸ or R⁹ together form a 5 or 6-membered cycloalkyl ring, a 5 or 6-membered fluorocycloalkyl ring, or a 5 or 6-membered oxygen containing heterocyclic ring;
   R⁶ and one of R¹⁰ or R¹¹ together form a 4 to 6-membered heterocyclyl ring or a 4 to 6-membered fluoroheterocyclyl ring;
   one of R⁸ or R⁹ and one of R¹² or R¹³ together form a 4 to 7-membered heterocyclyl ring or a 4 to 7-membered fluoroheterocyclyl ring;
   one of R⁸ or R⁹ and one of R¹⁵ or R¹⁶ together form a 5 to 7-membered heterocyclyl ring or a 5 to 7-membered fluoroheterocyclyl ring;
   one of R¹⁰ or R¹¹ and one of R¹² or R¹³ together form a 3 to 6-membered cycloalkyl ring, a 3 to 6-membered fluorocycloalkyl ring, or a 5 or 6-membered oxygen containing heterocyclic ring;
   one of R¹⁰ or R¹¹ and one of R¹⁵ or R¹⁶ together form a 5 or 6-membered cycloalkyl ring or a 5 or 6-membered fluorocycloalkyl ring;
   one of R⁸ or R⁹ and one of R¹⁰ or R¹¹ together form a 5 to 7-membered heterocyclyl ring or a 5 to 7-membered fluoroheterocyclyl ring; and/or
   one of R¹² or R¹³ and one of R¹⁵ or R¹⁶ together form a 3 to 6-membered cycloalkyl ring, a 3 to 6-membered fluorocycloalkyl ring, or a 5 or 6-membered oxygen containing heterocyclic ring.

In one embodiment of the first aspect, the present invention provides a compound of formula (I) or a pharmaceutically acceptable salt or prodrug thereof:

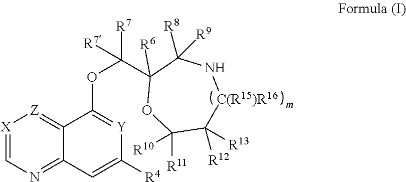

wherein:
Z is CR¹ or N;
Y is CH or N;
X is CR² or N; and
no more than one of X, Y or Z is N;
   wherein:
R¹ is selected from the group consisting of: hydrogen, C₁₋₆alkyl, C₁₋₆fluoroalkyl, C₂₋₆alkenyl, C₂₋₆fluoroalkenyl, C₂₋₆alkynyl, C₂₋₆fluoroalkynyl, C₃₋₆cycloalkyl, halo, —O—C₁₋₆alkyl, —O—C₁₋₆fluoroalkyl, —O—C₂₋₆alkenyl, —O—C₂₋₆fluoroalkenyl, —O—C₂₋₆alkynyl, —O—C₂₋₆fluoroalkynyl and cyano;
R² is selected from the group consisting of: hydrogen, C₁₋₆alkyl, C₁₋₆fluoroalkyl, C₂₋₆alkenyl, C₂₋₆fluoroalkenyl, C₂₋₆alkynyl, C₂₋₆fluoroalkynyl, halo, —O—C₁₋₆alkyl, —O—C₁₋₆fluoroalkyl, —O—C₂₋₆alkenyl, —O—C₂₋₆fluoroalkenyl, —O—C₂₋₆alkynyl, —O—C₂₋₆fluoroalkynyl and cyano;
R⁴ is 5-membered cycloalkene or 5-membered heteroaryl, each of which is optionally fused to form a 5:6, or 5:5 aromatic or heteroaromatic bicycle; wherein each R⁴ is optionally substituted by one or more R⁵; wherein each R⁵ is independently selected from the group consisting of: —R¹⁴, —R¹⁴-cycloalkyl-R¹⁹, —R¹⁴-cyclofluoroalkyl-R¹⁹, —R¹⁴-heterocyclyl-R¹⁹, —R¹⁴-fluoroheterocyclyl-R¹⁹, —R¹⁴-heteroaryl-R¹⁹, —R¹⁴-aryl-R¹⁹, -cycloalkyl-R¹⁹, -cyclofluoroalkyl-R¹⁹, -heterocyclyl-R¹⁹, -fluoroheterocyclyl-R¹⁹, -heteroaryl-R¹⁹, -aryl-R¹⁹, —R¹⁴—O—R⁹, Cl, F, cyano, —OR¹⁹, —SR¹⁹, —SOR¹⁹, —SO₂R¹⁹, —N(R¹⁹)₂, —N(R¹⁹)COR¹⁹, —CON(R¹⁹)₂, —N(R¹⁹)CON(R¹⁹)₂, —N(R¹⁹)COOR¹⁹, OCON(R¹⁹)₂, —N(R¹⁹)SO₂R¹⁹, —SO₂N(R¹⁹)₂, and ═O; wherein each R¹⁴ is independently selected from the group consisting of C₁₋₆alkyl, C₂₋₆alkenyl, C₂₋₆alkynyl, C₁₋₆fluoroalkyl, C₂₋₆fluoroalkenyl, C₂₋₆fluoroalkynyl and C₃₋₆cycloalkyl; wherein each R¹⁹ is independently selected from the group consisting of H, C₁₋₆alkyl, C₂₋₆alkenyl, C₂₋₆alkynyl, C₁₋₆fluoroalkyl, C₂₋₆fluoroalkenyl, C₂₋₆fluoroalkynyl and C₃₋₆cycloalkyl;
m is 0 or 1;
R⁶ is selected from the group consisting of: H, C₁₋₆alkyl, C₁₋₆fluoroalkyl, C₃₋₆cycloalkyl, C₃₋₆fluorocycloalkyl, C₂₋₆alkenyl, C₂₋₆fluoroalkenyl, C₂₋₆alkynyl and C₂₋₆fluoroalkynyl;
R⁷ and R^{7′} are independently selected from the group consisting of: H, fluoro, C₁₋₆alkyl, C₁₋₆fluoroalkyl, C₃₋₆cycloalkyl, C₃₋₆ fluorocycloalkyl, C₂₋₆alkenyl, C₂₋₆fluoroalkenyl, C₂₋₆alkynyl and C₂₋₆fluoroalkynyl; or R⁷ and R^{7′} together form a 3 to 6-membered cycloalkyl ring, a 3 to 6-membered fluorocycloalkyl ring, or a 4 to 6-membered oxygen containing heterocyclic ring;
R⁸ and R⁹ are independently selected from the group consisting of: H, C₁₋₆alkyl, C₁₋₆fluoroalkyl, C₃₋₆cycloalkyl, C₃₋₆fluorocycloalkyl, C₂₋₆alkenyl, C₂₋₆fluoroalkenyl, C₂₋₆alkynyl and C₂₋₆fluoroalkynyl; or R⁸ and R⁹ together form a 3 to 6-membered cycloalkyl ring, a 3 to 6-membered fluorocycloalkyl ring, or a 4 to 6-membered oxygen containing heterocyclic ring;
R¹⁰ and R¹¹ are independently selected from the group consisting of: H, C₁₋₆alkyl, fluoro, C₁₋₆fluoroalkyl, C₃₋₆cycloalkyl, C₃₋₆fluorocycloalkyl, C₂₋₆alkenyl, C₂₋₆fluoroalkenyl, C₂₋₆alkynyl and C₂₋₆fluoroalkynyl; or R¹⁰ and R¹¹ together form a 3 to 6-membered cycloalkyl ring, a 3 to 6-membered fluorocycloalkyl ring, or a 4 to 6-membered oxygen containing heterocyclic ring;
R¹² and R¹³ are independently selected from the group consisting of: H, C₁₋₆alkyl, fluoro, C₁₋₆fluoroalkyl, C₃₋₆cycloalkyl, C₃₋₆fluorocycloalkyl, C₂₋₆alkenyl, C₂₋₆fluoroalkenyl, C₂₋₆alkynyl and C₂₋₆fluoroalkynyl; or R¹² and R¹³ together form a 3 to 6-membered cycloalkyl ring, a 3 to 6-membered fluorocycloalkyl ring, or a 4 to 6-membered oxygen containing heterocyclic ring;
R¹⁵ and R¹⁶ are independently selected from the group consisting of: H, C₁₋₆alkyl, C₁₋₆fluoroalkyl, C₃₋₆cycloalkyl, C₃₋₆fluorocycloalkyl, C₂₋₆alkenyl, C₂₋₆fluoroalkenyl, C₂₋₆alkynyl and C₂₋₆fluoroalkynyl; or R¹⁵ and R¹⁶ together form a 3 to 6-membered cycloalkyl ring, a 3 to 6-membered fluorocycloalkyl ring, or a 4 to 6-membered oxygen containing heterocyclic ring; or wherein:
   one of R⁷ or R^{7′} and one of R⁸ or R⁹ together form a 5 or 6-membered cycloalkyl ring, a 5 or 6-membered fluorocycloalkyl ring, or a 5 or 6-membered oxygen containing heterocyclic ring;
   one of R⁷ or R^{7′} and R⁶ together form a 3 to 6-membered cycloalkyl ring, a 3 to 6-membered fluorocycloalkyl ring, or a 4 to 6-membered oxygen containing heterocyclic ring;
   R⁶ and one of R⁸ or R⁹ together form a 5 or 6-membered cycloalkyl ring, a 5 or 6-membered fluorocycloalkyl ring, or a 5 or 6-membered oxygen containing heterocyclic ring;
   R⁶ and one of R¹⁰ or R¹¹ together form a 4 to 6-membered heterocyclyl ring or a 4 to 6-membered fluoroheterocyclyl ring;
   one of R⁸ or R⁹ and one of R¹⁰ or R¹¹ together form a 4 to 7-membered heterocyclyl ring or a 4 to 7-membered fluoroheterocyclyl ring;
   one of R⁸ or R⁹ and one of R¹⁵ or R¹⁶ together form a 5 to 7-membered heterocyclyl ring or a 5 to 7-membered fluoroheterocyclyl ring;
   one of R¹⁰ or R¹¹ and one of R¹² or R¹³ together form a 3 to 6-membered cycloalkyl ring, a 3 to 6-membered fluorocycloalkyl ring, or a 5 or 6-membered oxygen containing heterocyclic ring;
   one of R¹⁰ or R¹¹ and one of R¹⁵ or R¹⁶ together form a 5 or 6-membered cycloalkyl ring or a 5 or 6-membered fluorocycloalkyl ring;
   one of R⁸ or R⁹ and one of R¹⁰ or R¹¹ together form a 5 to 7-membered heterocyclyl ring or a 5 to 7-membered fluoroheterocyclyl ring; and/or
   one of R¹² or R¹³ and one of R¹⁵ or R¹⁶ together form a 3 to 6-membered cycloalkyl ring, a 3 to 6-membered fluorocycloalkyl ring, or a 5 or 6-membered oxygen containing heterocyclic ring.

In one embodiment, the compound of Formula (I) is a compound of Formula (II):

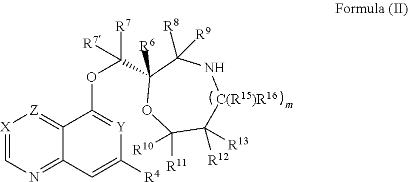

In a further embodiment, the compound of Formula (I) is a compound of Formula (III):

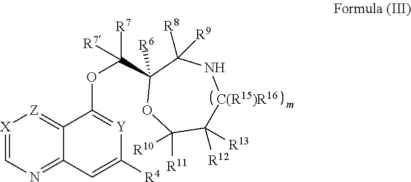

Advantageously, the inventors have found that compounds of Formula (I) may provide potent Syk inhibitors.

Syk is known to play multiple roles in Alzheimer's Disease (AD) pathologies, primarily in microglia, the immune cells of the brain. Neuroinflammation is a key driver of AD pathology. Syk promotes neuroinflammation (Ye et al. 2020; Zeng et al. 2014) and acts as a pro-inflammatory signalling mediator for receptors such as TREM2, DAP12, Toll-like receptors (TLR) and Fe-receptors, which have all been correlated to, or are upregulated in AD brains (Fuller, Stavenhagen, and Teeling 2014; Nizami et al. 2019; Mielcarska et al. 2019; Guerreiro et al. 2013). β-amyloid plaques and Tau hyperphosphorylation in neurons are the primary hallmarks of AD and both are affected by Syk modulation in vitro and in mouse models (Schweig et al. 2017; Paris et al. 2014; Schweig et al. 2019). Syk mediates the chronic pro-inflammatory microglial response to β-amyloid and can also increase the production of β-amyloid in neurons. Syk directly phosphorylates Tau and co-localises with Tau in mouse neurons. Thus, in addition to its key role in neuroinflammation, Syk is involved in the generation and activation of β-amyloid and Tau, which lead to the archetypal AD pathologies.

Syk is also implicated in other neuroinflammatory-driven diseases including other types of dementia, Parkinson's disease (PD), multiple sclerosis (MS), stroke, traumatic brain injury (TBI) and subarachnoid hemorrhage (SAH). In MS, Syk has a dual effect of modulating both the peripheral (T-cells, B-cells) and CNS (B-cells, microglia, macrophages, T-cells) immune responses, integral in the pathology of MS (Baecher-Allan, Kaskow, and Weiner 2018). Syk has been identified as a key mediator of neuroinflammation following stroke and its inhibition has been shown to reduce early post-reperfusion inflammation, resulting in improved recovery post-infarction (Ye et al. 2020). Syk inhibition has also been shown to improve neurological function in a rat model of TBI (He et al. 2015). Syk is implicated in both TBI (Morin, Front Aging Neurosci, 2018) and SAH (He, Stroke, 2015) in animal models.

Syk is correlated to AD, vascular dementia and other neuroinflammatory related diseases such as multiple sclerosis via genome-wide association studies (Sierksma et al. 2020; Disanto et al. 2014; Kim, Kong, and Lee 2013; Ryu et al. 2014; International Multiple Sclerosis Genetics Consortium et al. 2011), highlighting the fundamental role of Syk in neuroinflammation. Further validating Syk as a target for AD, the expression and activation of Syk are increased in AD patient brains, particularly in degenerating neurites associated with β-amyloid plaques (Ghosh and Geahlen 2015; Schweig et al. 2017). Importantly, the up-regulation of Syk expression and activation that is observed in the brains of human AD patients is reflected in mouse models of the disease. Three mouse models of AD (one that focusses on Tau pathology and two that are based on β-amyloid pathology) display an age-dependent increase in both the expression and activation of Syk in neurons, dystrophic neurites or microglia in the brain (Schweig et al. 2017; Sierksma et al. 2020), which correlates to the human condition.

It has been found that Syk kinase signalling regulates neuroinflammatory responses and immune activation in response to pathological protein aggregates found in PD and AD. Selective inhibition of the Syk kinase pathway could therefore offer a multiple-pronged approach to treating PD and AD; targeting the disease-modifying pathways of 1) tau phosphorylation, 2) amyloid beta production and 3) neuroinflammation. The potential of inhibition of the Syk kinase pathway may be of therapeutic benefit to other neuroinflammatory diseases such as stroke, and multiple sclerosis.

PD and AD are extremely debilitating due to significant disability, dysfunction and duration and there is an overwhelming unmet need for therapeutics that can alter disease progression. The current approved treatments for PD and AD provide only symptomatic benefit and there are no disease-modifying drugs available for patients. There has been a high failure rate from agents targeting a single pathway in AD, most notably the β-amyloid-targeting antibodies and β-secretase (BACE) inhibitors. Due to the heterogeneity of these diseases, a mechanism of action that addresses multiple pathogenic pathways in a targeted population has higher potential for translation.

Accordingly, Syk inhibitors that can penetrate the blood brain barrier and have effect in the Central Nervous System could be potentially used to treat neurological disorders including AD, PD and MS.

However, to the inventors' knowledge Syk has not been pursued for Central Nervous System (CNS) conditions and based on their extensive benchmarking, the current clinical compounds do not reach adequate concentrations in the brain to inhibit Syk. Furthermore, to the inventors' knowledge small molecule Syk inhibitors which are sufficiently potent and brain penetrant have not been described in the literature.

To the inventors' knowledge there are very few, if any, disclosures in the literature of brain penetrant small molecule Syk inhibitors to treat AD. Relative to other largely unsuccessful targets tested in AD, such as β-amyloid or Tau-targeting biologics, Syk has compelling advantages. Rather than targeting only one pathologic process involved in AD, Syk is involved in multiple disease pathways. In addition to directly reducing neuroinflammation, Syk inhibition reduces β-amyloid production and Tau hyperphosphorylation.

In one embodiment, compounds of Formula (I) may be capable of penetrating the blood brain barrier. In one embodiment, compounds of Formula (I) may be capable of acting on the Central Nervous System in vivo.

Oral administration of a small molecule Syk inhibitor that can cross the blood-brain barrier would have clear advantages in its direct mechanism of action against AD, PD and MS pathologies, ease of use, patient compliance and cost. However, it has been traditionally very difficult to achieve the design and synthesis of brain-penetrating kinase inhibitors because of the nature of the types of molecules that bind potently to the active site of a kinase enzyme. The types of molecules that are typically found to be potent kinase inhibitors have high molecular weights, high polar surface areas and too many H-bond donors or acceptors. For a good CNS drug it is necessary to have a low molecular weight (lower than 500, preferably lower than 450 and most preferably less than 400 Daltons), a low polar surface area (less than 120 Å², preferably less than 100 Å² and most preferably less than 80 Å²), a log P between 2 and 5 (most preferably between 2 and 4) and three or less H-bond donors (preferably two or less, more preferably one or less H-bond donors) and less than 10H-bond acceptors (preferably less than 8 and most preferably less than 6H-bond acceptors) (Hitchcock et. al, 2006). To the inventors' knowledge, a Syk inhibitor meeting the above criteria which achieves sufficient brain penetration of free drug is not described in the literature. In some embodiments of compounds of Formula (I), (II) or (III), the inventors have surprisingly been able to achieve potent brain-penetrant small molecule Syk inhibitors that have direct applicability to treat AD and many other neurological diseases and peripheral conditions.

In one embodiment, compounds of the present invention provide a CNS penetrant, selective antagonist that is suitable for oral administration, which may be used to treat a neurological disease or disorder such as AD, PD or MS.

By inhibiting Syk, the present invention may offer the advantage of simultaneously modulating multiple pathways in AD, with reduction of neuroinflammation at the forefront and additional effects on Tau activation and β-amyloid formation. The proposed therapy is intended to reduce the likelihood that the condition will progress to a more advanced stage of disease. In AD, differentiation over approved agents, e.g. Aricept and Exelon, which only address symptoms, is increased efficacy and disease-modifying potential. Differentiation over β-amyloid and Tau targeting agents in clinical development is increased efficacy, by targeting multiple disease pathologies. Oral administration also provides an advantage over biologics.

In some embodiments of compounds of Formula (I), Formula (II) or Formula (III), one or more of the features of paragraphs [0028] to [0059] may apply (the features of paragraphs [0028] to [0059] may apply alone or in combination with features of any others of paragraphs [0028] to [0059]). For the avoidance of doubt, any of the definitions of Z, Y, X, R¹, R², R⁴, R⁵, R⁶, R⁷, R^{7′}, R⁸, R⁹, R¹⁰, R¹¹, R¹², R¹³, R¹⁵ and R¹⁶ may be combined with any other definitions of Z, Y, X, R¹, R², R⁴, R⁵, R⁶, R⁷, R^{7′}, R⁸, R⁹, R¹⁰, R¹¹, R¹², R¹³, R¹⁵ and R¹⁶ described herein.

In one embodiment, Z is CR¹. In another embodiment, Z is N.

In another embodiment, Y is CH. In a further embodiment. Y is N.

In one embodiment, X is CH. In another embodiment, X is N.

In one embodiment, R is selected from the group consisting of: hydrogen, C₁₋₆alkyl, —O C₁₋₆alkyl and C₁₋₆fluoroalkyl; especially hydrogen, C₁₋₆alkyl and C₁₋₆fluoroalkyl. In one embodiment R¹ is H or C₁₋₆alkyl; especially R¹ is H or CH₃; more especially R¹ is H.

In one embodiment, R² is selected from the group consisting of: hydrogen, C₁₋₆alkyl, C₁₋₆fluoroalkyl, C₂₋₆alkenyl and C₂₋₆fluoroalkenyl; especially R² is H, C₁₋₆alkyl or C₂₋₆alkenyl; more especially R is H, CH₃ or —CF=CH₂; most especially R² is H.

In one embodiment, Z is N, Y is CH and X is CR²; especially Z is N, Y is CH, and X is CH, C—C₁₋₆alkyl or C—C₂₋₆alkenyl; more especially Z is N, Y is CH, and X is CH.

In another embodiment, Y is N, Z is CR¹ and X is CR²; especially Y is N, X is CH, C—C₁₋₆alkyl or C—C₂₋₆alkenyl, and Z is CH, C—C₁₋₆fluoroalkyl, —C—O—C₁₋₆alkyl or C—C₁₋₆alkyl; more especially Y is N, X is CH, C—CH₃ or C—CH═CH₂, and Z is CH, C—CF, C—CH₃, C—CH₂—CH₃, or C—O—CH₃; most especially Y is N, X is CH, and Z is CH.

In a further embodiment, X is N, Y is CH and Z is CR¹; especially X is N, Y is CH, and Z is CH or C—C₁₋₆alkyl; more especially X is N, Y is CH, and Z is CH or C—CH₃; most especially X is N, Y is CH, and Z is CH.

In another embodiment, X is CR², Y is CH and Z is CR¹; especially X is CH, C—C₁₋₆alkyl or C—C₂₋₆alkenyl, Y is CH, and Z is CH or C—C₁₋₆alkyl; more especially X is CH, C—CH₃ or C—CH═CH₂, Y is CH, and Z is CH or C—CH₃; most especially X is CH, Y is CH, and Z is CH.

In one embodiment, R⁴ is 5-membered heteroaryl, which is optionally fused to form a 5:6, or 5:5 aromatic bicycle; wherein R⁴ is optionally substituted by one or more R.

In one embodiment, R⁴ is 5-membered cycloalkene or 5-membered heteroaryl, each of which is optionally fused to form a 5:6, or 5:5 aromatic or heteroaromatic bicycle; wherein each R⁴ is optionally substituted by one or more R.

In a further embodiment, R⁴ is selected from the group consisting of: cyclopentenyl, pyrrolyl, 2,3-dihydro-pyrrolizinyl, pyrazolyl, 4,5,6,7-tetrahydropyrazolo[1,5-a]pyridinyl, thiophenyl, 1,2-oxazolyl, 1,3-thiazolyl, and 1,2-thiazolyl; wherein said R⁴ groups are optionally substituted by one or more R⁵. In a further embodiment, R⁴ is selected from the group consisting of: pyrrolyl and pyrazolyl; wherein said R⁴ groups are optionally substituted by one or more R⁵.

In another embodiment, R⁴ is selected from the group consisting of:

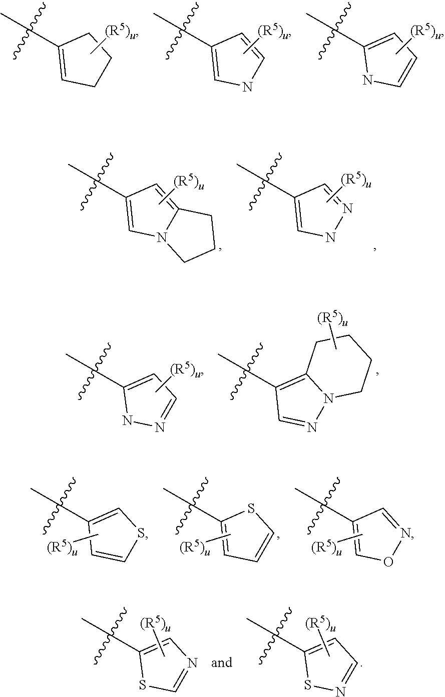

In a further embodiment, R⁴ is selected from the group consisting of:

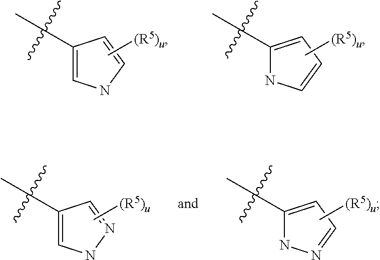

especially R⁴ is selected from the group consisting of:

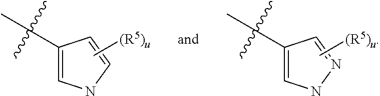

In the aforementioned embodiments, u is an integer from 0 to the maximum number of substitution positions on said group (especially 0, 1 or 2; more especially 0 or 1).

In one embodiment, each R⁵ is independently selected from the group consisting of: —R¹⁴, —R¹⁴-cycloalkyl-R¹⁹, —R¹⁴-cyclofluoroalkyl-R¹⁹, —R¹⁴-heterocyclyl-R¹⁹, —R¹⁴-fluoroheterocyclyl-R¹⁹, —R¹⁴-heteroaryl-R¹⁹, —R¹⁴-aryl-R¹⁹, -cycloalkyl-R¹⁹, -cyclofluoroalkyl-R¹⁹, -heterocyclyl-R¹⁹, -fluoroheterocyclyl-R¹⁹, -heteroaryl-R¹⁹, -aryl-R¹⁹, —R¹⁴—O—R¹⁹, Cl, F, cyano, —OR¹⁹, —SR¹⁹, —SOR¹⁹, —SO₂R¹⁹, —N(R¹⁹)₂, —N(R¹⁹)COR¹⁹, —CON(R¹⁹)₂, —N(R¹⁹)CON(R¹⁹)₂, —N(R¹⁹)COOR¹⁹, —OCON(R¹⁹)₂, —N(R¹⁹)SO₂R¹⁹, —SO₂N(R¹⁹)₂, and ═O; wherein each R⁴ is independently selected from the group consisting of C₁₋₆alkyl, C₂₋₆alkenyl, C₂₋₆alkynyl, C₁₋₆fluoroalkyl, C₂₋₆fluoroalkenyl, C₂₋₆fluoroalkynyl and C₃₋₆cycloalkyl; wherein each R¹⁹ is independently selected from the group consisting of H, C₁₋₆alkyl, C₂₋₆alkenyl, C₂₋₆alkynyl, C₁₋₆fluoroalkyl, C₂₋₆fluoroalkenyl, C₂₋₆fluoroalkynyl and C₃₋₆cycloalkyl. In another embodiment, each R⁵ is independently selected from the group consisting of: —R¹⁴, —R¹⁴-cycloalkyl-R¹⁹, —R¹⁴-cyclofluoroalkyl-R¹⁹, —R¹⁴-heterocyclyl-R¹⁹, —R¹⁴-fluoroheterocyclyl-R¹⁹, —R¹⁴-heteroaryl-R¹⁹, —R¹⁴-aryl-R¹⁹, -cycloalkyl-R¹⁹, -cyclofluoroalkyl-R¹⁹, -heterocyclyl-R¹⁹, -fluoroheterocyclyl-R¹⁹, -heteroaryl-R¹⁹, -aryl-R¹⁹, —R¹⁴—O—R¹⁹, Cl, F, cyano, —OR¹⁹, —SR¹⁹ and ═O; wherein each R¹⁴ is independently selected from the group consisting of C₁₋₆alkyl, C₂₋₆alkenyl, C₂₋₆alkynyl, C₁₋₆fluoroalkyl, C₂₋₆fluoroalkenyl and C₂₋₆fluoroalkynyl; wherein each R¹⁹ is independently selected from the group consisting of H, C₁₋₆alkyl, C₂₋₆alkenyl, C₂₋₆alkynyl, C₁₋₆fluoroalkyl, C₂₋₆fluoroalkenyl and C₂₋₆fluoroalkynyl.

In another embodiment, each R⁵ is independently selected from the group consisting of: —R¹⁴, —R¹⁴-cycloalkyl-R¹⁹, —R¹⁴-cyclofluoroalkyl-R¹⁹, —R¹⁴-heterocyclyl-R¹⁹, —R¹⁴-fluoroheterocyclyl-R¹⁹, -cycloalkyl-R¹⁹, -cyclofluoroalkyl-R¹⁹, -heterocyclyl-R¹⁹, -fluoroheterocyclyl-R¹⁹, —R¹⁴—O—R¹⁹, Cl, F, cyano, —OR¹⁹, —SR¹⁹ and ═O; wherein each R¹⁴ is independently selected from the group consisting of C₁₋₆alkyl, C₂₋₆alkenyl, C₂₋₆alkynyl, C₁₋₆fluoroalkyl, C₂₋₆fluoroalkenyl and C₂₋₆fluoroalkynyl; wherein each R¹⁹ is independently selected from the group consisting of: H, C₁₋₆alkyl, C₂₋₆alkenyl, C₂₋₆alkynyl, C₁₋₆fluoroalkyl, C₂₋₆fluoroalkenyl and C₂₋₆fluoroalkynyl. In a further embodiment, each R⁵ is independently selected from the group consisting of: —R¹⁴, -R¹⁴cycloalkyl-R¹⁹, —R¹⁴-cyclofluoroalkyl-R¹⁹, —R¹⁴-heterocyclyl-R¹⁹, -cycloalkyl-R¹⁹, -cyclofluoroalkyl-R¹⁹, -heterocyclyl-R¹⁹, —R¹⁴—O—R¹⁹, Cl and ═O; wherein each R¹⁴ is independently selected from the group consisting of C₁₋₆alkyl, C₂₋₆alkenyl, C₂₋₆alkynyl, C₁₋₆fluoroalkyl, C₂₋₆fluoroalkenyl and C₂₋₆fluoroalkynyl; wherein each R¹⁹ is independently selected from the group consisting of H, C₁₋₆alkyl, C₂₋₆alkenyl, C₂₋₆alkynyl, C₁₋₆fluoroalkyl, C₂₋₆fluoroalkenyl and C₂₋₆fluoroalkynyl. In yet another embodiment, each R⁵ is independently selected from the group consisting of: —R¹⁴, —R¹⁴-cycloalkyl-R¹⁹, —R¹⁴-cyclofluoroalkyl-R¹⁹, —R¹⁴-heterocyclyl-R¹⁹, -cycloalkyl-R¹⁹, -cyclofluoroalkyl-R¹⁹, -heterocyclyl-R¹⁹, —R¹⁴—O—R¹⁹, Cl and ═O; wherein each R¹⁴ is independently selected from the group consisting of C₁₋₆alkyl, C₂₋₆alkenyl, C₁₋₆fluoroalkyl and C₂₋₆fluoroalkenyl; wherein each R¹⁹ is independently selected from the group consisting of H, C₁₋₆alkyl, C₂₋₆alkenyl, C₁₋₆fluoroalkyl and C₂₋₆fluoroalkenyl.

In one embodiment, each R⁵ is independently selected from the group consisting of: —C₁₋₆alkyl, —C₁₋₆fluoroalkyl, —C₂₋₆fluoroalkenyl, —C₁₋₆alkyl-cycloalkyl, —C₁₋₆alkyl-cyclofluoroalkyl, —C₁₋₆alkyl-heterocyclyl, -cycloalkyl, -cyclofluoroalkyl, -cycloalkyl-C₁₋₆alkyl, —C₁₋₆alkyl-O—C₁₋₆alkyl, -heterocyclyl, -heterocyclyl-C₂₋₆alkyl, ═O and Cl. In a further embodiment, each R⁵ is independently selected from the group consisting of: —C₁₋₆alkyl and -heterocyclyl.

In one embodiment, R⁴ is selected from the group consisting of:

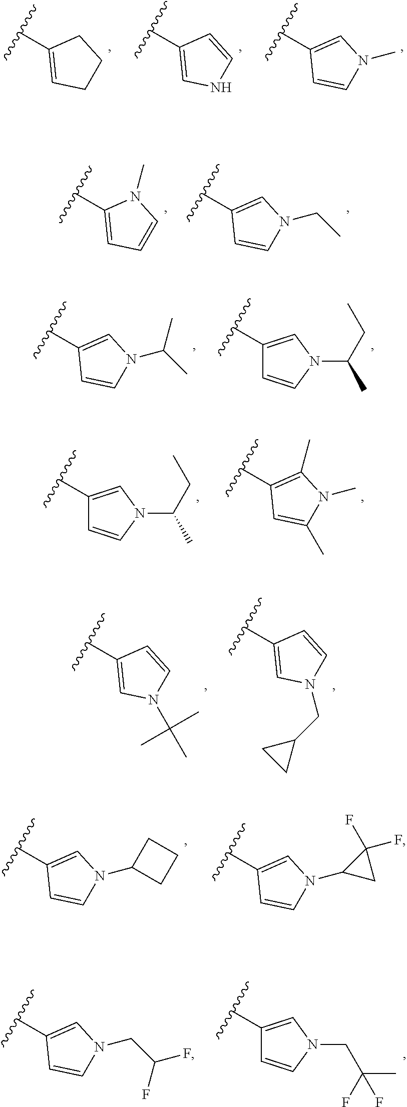

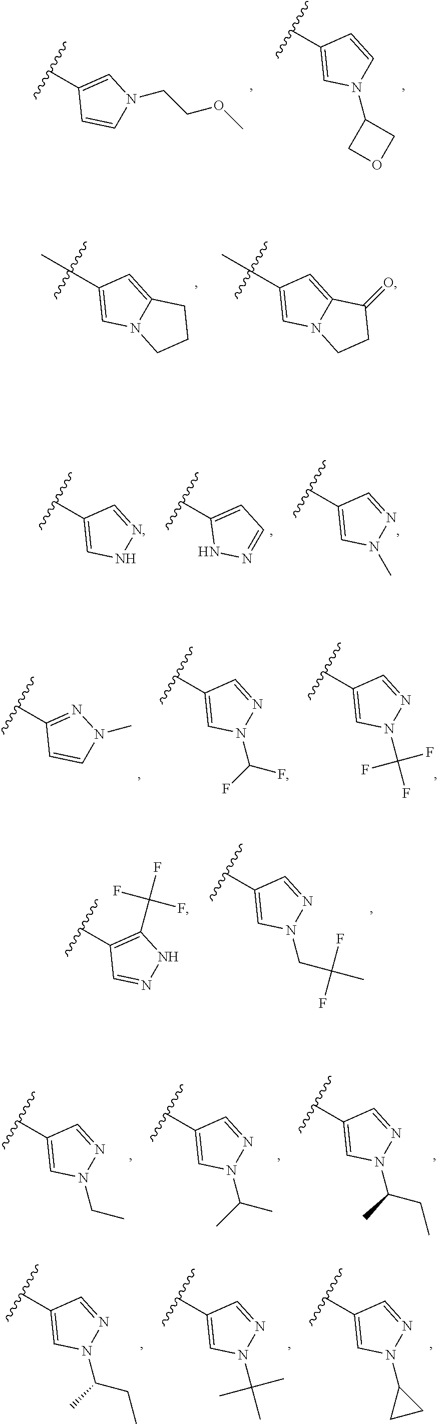

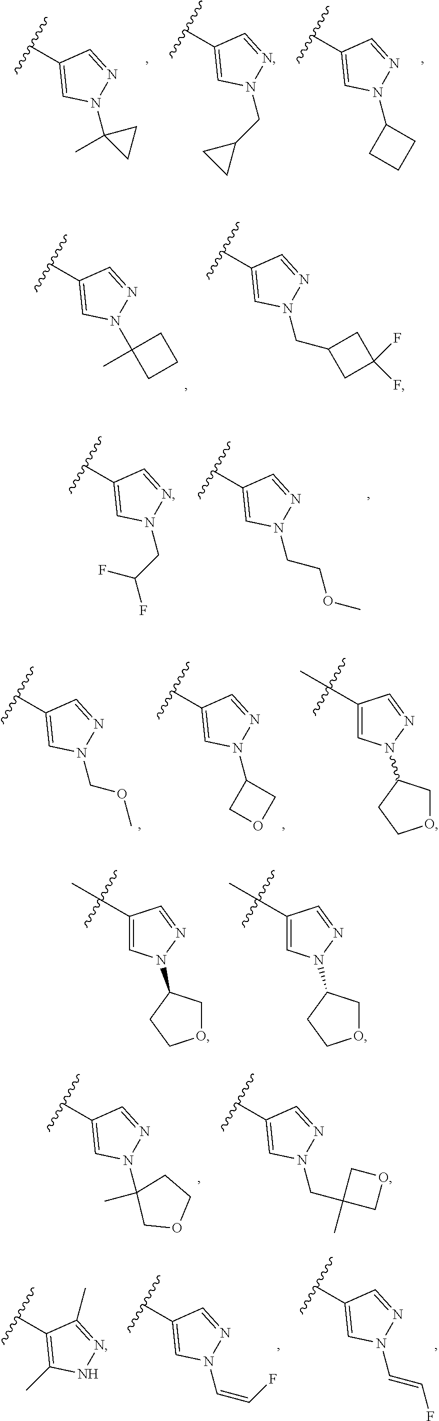

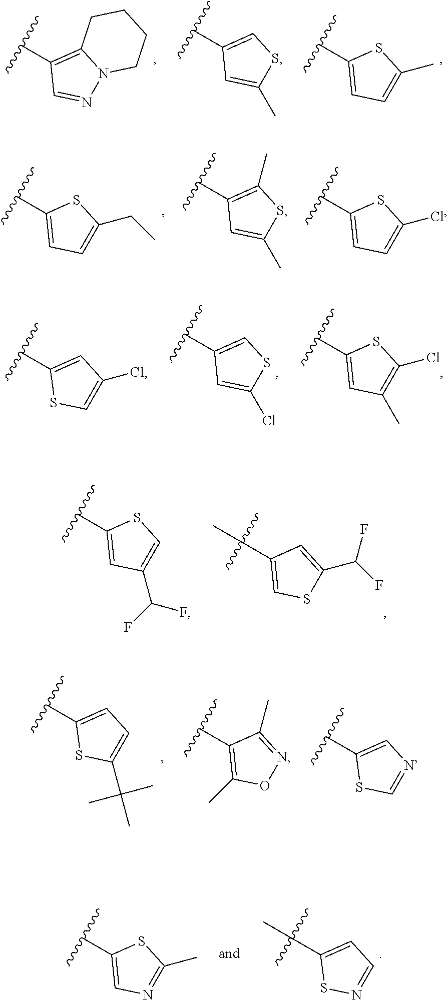

In another embodiment, R⁴ is selected from the group consisting of:

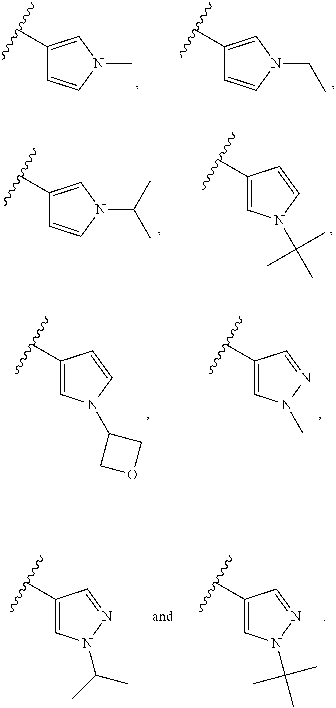

In one embodiment, R⁶ is selected from the group consisting of H, C₁₋₆alkyl, C₁₋₆fluoroalkyl, C₂₋₆alkenyl, C₂₋₆fluoroalkenyl, C₂₋₆alkynyl and C₂₋₆fluoroalkynyl; especially selected from the group consisting of H, C₁₋₆alkyl and C₁₋₆fluoroalkyl; most especially selected from the group consisting of H and C₁₋₆alkyl. In one embodiment, R⁶ is selected from the group consisting of H, CH₃ and CH₂—CH₃. In another embodiment, R⁶ is H.

In one embodiment, R⁷ and R^{7′} are independently selected from the group consisting of: H, C₂₋₆alkyl, C₂₋₆fluoroalkyl, C₂₋₆alkenyl, C₂₋₆fluoroalkenyl, C₂₋₆alkynyl and C₂₋₆fluoroalkynyl. In another embodiment, R⁷ and R are independently selected from the group consisting of: H, fluoro, C₁₋₆alkyl, C₁₋₆fluoroalkyl, C₃₋₆cycloalkyl, C₃₋₆fluorocycloalkyl, C₂₋₆alkenyl, C₂₋₆fluoroalkenyl, C₂₋₆alkynyl and C₂₋₆fluoroalkynyl; especially H, C₁₋₆alkyl and C₁₋₆fluoroalkyl; more especially H and C₁₋₆alkyl. In a further embodiment, R⁷ and R^{7′} are independently selected from the group consisting of H, CH₃ and CF₃; more especially H and CH₃; most especially H. In one embodiment, at least one of R⁷ and R^{7′} are H.

In one embodiment, R¹ and R⁹ are independently selected from the group consisting of: H, C₁₋₆alkyl, C₁₋₆fluoroalkyl, C₃₋₆cycloalkyl, C₃₋₆fluorocycloalkyl, C₂₋₆alkenyl, C₂₋₆fluoroalkenyl, C₂₋₆alkynyl and C₁₋₆fluoroalkynyl; especially H, C₁₋₆alkyl, C₁₋₆fluoroalkyl, C₂₋₆alkenyl, C₂₋₆fluoroalkenyl, C₂₋₆alkynyl and C₂₋₆fluoroalkynyl. In a further embodiment, R⁸ and R⁹ are independently selected from the group consisting of: H, C₁₋₆alkyl and C₁₋₆fluoroalkyl; especially H and C₁₋₆alkyl. In one embodiment, R⁸ and R⁹ are independently selected from the group consisting of: H, CH₃ and CF₃; more especially H and CH₃; most especially H. In one embodiment, at least one of R⁸ and R⁹ are H.

In one embodiment, R¹⁰ and R¹¹ are independently selected from the group consisting of: H, C₁₋₆alkyl, fluoro, C₁₋₆fluoroalkyl, C₃₋₆cycloalkyl, and C₃₋₆fluorocycloalkyl; or R¹⁰ and R¹¹ together form a 3 to 6-membered cycloalkyl ring, a 3 to 6-membered fluorocycloalkyl ring, or a 4 to 6-membered oxygen containing heterocyclic ring (such as oxetane, tetrahydrofuran or pyran). In another embodiment, R¹⁰ and R¹¹ are independently selected from the group consisting of: H, C₁₋₆alkyl, fluoro and C₁₋₆fluoroalkyl; or R¹⁰ and R¹¹ together form a 3 to 6-membered cycloalkyl ring, a 3 to 6-membered fluorocycloalkyl ring, or a 4 to 6-membered oxygen containing heterocyclic ring. In a further embodiment, R¹⁰ and R¹¹ are independently selected from the group consisting of: H, C₁₋₆alkyl and fluoro; or R¹⁰ and R¹¹ together form a 3 to 6-membered cycloalkyl ring, or a oxetane, tetrahydrofuran or pyran ring. In another embodiment, R¹⁰ and R¹¹ are independently selected from the group consisting of: H, CH₃, —CH₂—CH₃, F, CH₂F, CHF₂ and CF₃; or R¹⁰ and R¹¹ together form a cyclopropyl, cyclobutyl or oxetanyl ring. In another embodiment, R¹⁰ and R¹¹ are independently selected from the group consisting of: H, CH₃, —CH₂—CH₃, —CH—(CH₃)₂, —C—(CH₃)₃, F, cyclopropyl, CHF₂ and CF₃; or R¹⁰ and R¹¹ together form a cyclopropyl, cyclobutyl or oxetanyl ring. In a further embodiment, R¹⁰ and R¹¹ are independently selected from the group consisting of: H, CH₃, —CH₂—CH₃ and CHF₂; or R¹⁰ and R¹¹ together form a cyclopropyl or cyclobutyl ring. In one embodiment, both R¹⁰ and R¹¹ are CH₃. In another embodiment, both R¹⁰ and R¹¹ are H.

In one embodiment, R¹² and R¹³ are independently selected from the group consisting of: H, C₁₋₆alkyl, fluoro, C₁₋₆fluoroalkyl, C₃₋₆cycloalkyl, C₃₋₆fluorocycloalkyl, C₂₋₆alkenyl, C₂₋₆fluoroalkenyl, C₂₋₆alkynyl and C₂₋₆fluoroalkynyl; especially independently selected from the group consisting of: H, C₁₋₆alkyl, fluoro, C₁₋₆fluoroalkyl, C₂₋₆alkenyl, C₂₋₆fluoroalkenyl, C₂₋₆alkynyl and C₂₋₆fluoroalkynyl; more especially independently selected from the group consisting of: H, C₁₋₆alkyl, fluoro and C₁₋₆fluoroalkyl; most especially independently selected from the group consisting of: H, C₁₋₆alkyl and fluoro. In one embodiment, R¹² and R¹³ are independently selected from the group consisting of: H, CH₃ and F. In another embodiment both R¹² and R¹³ are H.

In one embodiment, m is 0. In another embodiment, m is 1.

In one embodiment, R¹⁵ and R¹⁶ are independently selected from the group consisting of: H, C₁₋₆alkyl, C₁₋₆fluoroalkyl, C₃₋₆cycloalkyl, C₃₋₆fluorocycloalkyl, C₂₋₆alkenyl, C₂₋₆fluoroalkenyl, C₂₋₆alkynyl and C₂₋₆fluoroalkynyl; especially independently selected from the group consisting of: H, C₁₋₆alkyl, C₁₋₆fluoroalkyl, C₂₋₆alkenyl, C₂₋₆fluoroalkenyl, C₂₋₆alkynyl and C₂₋₆fluoroalkynyl; more especially independently selected from the group consisting of: H, C₁₋₆alkyl and C₁₋₆fluoroalkyl; most especially independently selected from the group consisting of: H and C₁₋₆alkyl. In one embodiment, R¹⁵ aid R¹⁶ are independently selected from the group consisting of: H and CH₃. In another embodiment both R¹⁵ and R¹⁶ are H.

In one embodiment, one of R¹⁰ or R¹¹ and one of R¹² or R¹³ together may form a 3 to 6-membered cycloalkyl ring or fluorocycloalkyl ring. In one embodiment, one of R¹⁰ or R¹¹ and one of R¹² or R¹³ together may form a cyclopropyl, cyclobutyl or cyclopentyl ring; especially a cyclopropyl or cyclopentyl ring.

In one embodiment, one of R⁸ or R⁹ and one of R¹⁰ or R¹¹ together may form a 5 to 7-membered heterocyclyl ring or fluoroheterocyclyl ring. In one embodiment, one of R⁸ or R⁹ and one of R¹⁰ or R¹¹ together may be —CH₂—, —CH₂—CH₂—, or —CH₂—CH₂—CH₂—.

In one embodiment, one of R⁸ or R⁹ and one of R¹² or R¹³ together may form a 4 to 7-membered heterocyclyl ring or fluoroheterocyclyl ring. In one embodiment, one of R⁸ or R⁹ and one of R¹² or R¹³ together may be —CH₂—, —CH₂—CH₂—, or —CH₂—CH₂—CH₂—; especially —CH₂—. In one embodiment, if m is 1, then one of R⁸ or R⁹ and one of R¹² or R¹³ together may be a bond (this would form a four membered ring).

In one embodiment, one of R⁷ or R^{7′} and one of R⁸ or R⁹ together may form a 5 or 6-membered cycloalkyl ring or fluorocycloalkyl ring. In one embodiment, one of R⁷ or R^{7′} and one of R⁸ or R⁹ together may form a cyclopentyl or cyclohexyl ring; especially a cyclopentyl ring.

In one embodiment:
one of R⁷ or R^{7′} and one of R⁸ or R⁹ together form a 5 or 6-membered cycloalkyl ring, a 5 or 6-membered fluorocycloalkyl ring, or a 5 or 6-membered oxygen containing heterocyclic ring;
one of R⁷ or R^{7′} and R⁶ together form a 3 to 6-membered cycloalkyl ring, a 3 to 6-membered fluorocycloalkyl ring, or a 4 to 6-membered oxygen containing heterocyclic ring;
R⁶ and one of R⁸ or R⁹ together form a 5 or 6-membered cycloalkyl ring, a 5 or 6-membered fluorocycloalkyl ring, or a 5 or 6-membered oxygen containing heterocyclic ring;
R⁶ and one of R¹⁰ or R¹¹ together form a 4 to 6-membered heterocyclyl ring or a 4 to 6-membered fluoroheterocyclyl ring;
one of R⁸ or R⁹ and one of R¹² or R¹³ together form a 4 to 7-membered heterocyclyl ring or a 4 to 7-membered fluoroheterocyclyl ring;
one of R⁸ or R⁹ and one of R¹⁵ or R¹⁶ together form a 5 to 7-membered heterocyclyl ring or a 5 to 7-membered fluoroheterocyclyl ring;
one of R¹⁰ or R¹¹ and one of R¹² or R¹³ together form a 3 to 6-membered cycloalkyl ring, a 3 to 6-membered fluorocycloalkyl ring, or a 5 or 6-membered oxygen containing heterocyclic ring;
one of R¹⁰ or R¹¹ and one of R¹⁵ or R¹⁶ together form a 5 or 6-membered cycloalkyl ring or a 5 or 6-membered fluorocycloalkyl ring;
one of R⁸ or R⁹ and one of R¹⁰ or R¹¹ together form a 5 to 7-membered heterocyclyl ring or a 5 to 7-membered fluoroheterocyclyl ring; or
one of R¹² or R¹³ and one of R¹⁵ or R¹⁶ together form a 3 to 6-membered cycloalkyl ring, a 3 to 6-membered fluorocycloalkyl ring, or a 5 or 6-membered oxygen containing heterocyclic ring.

In one embodiment of the compound of Formula (T),

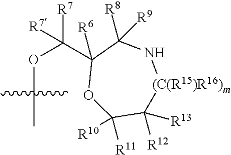

is selected from the group consisting of:

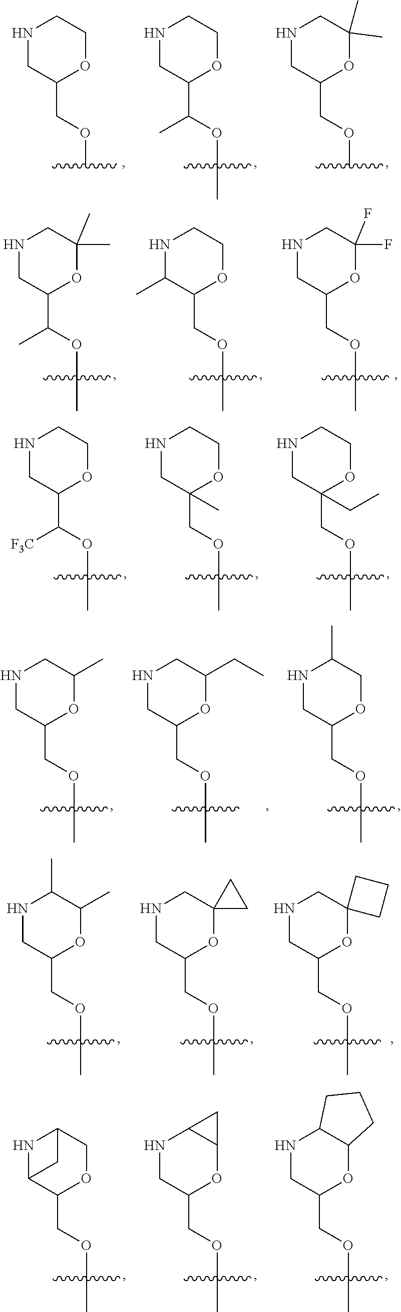

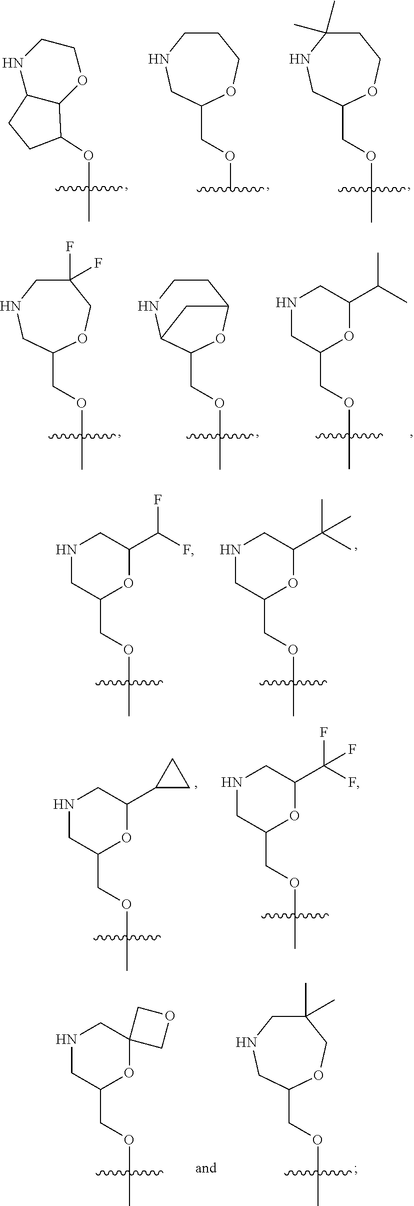

especially selected from the group consisting of:

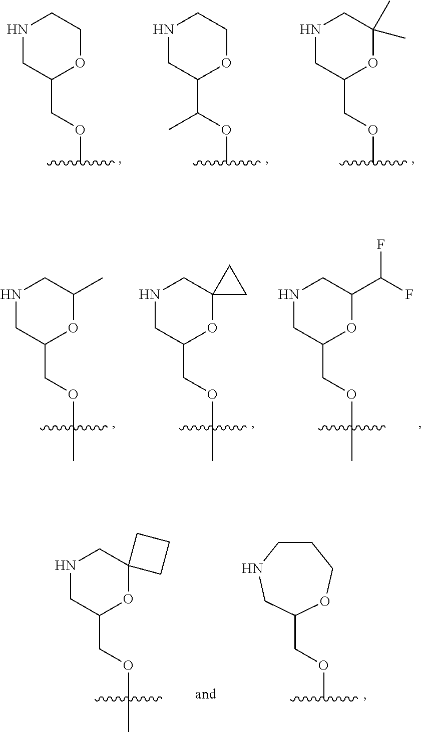

In one embodiment of the compound of Formula (I),

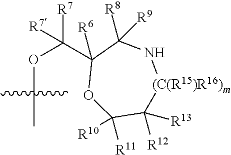

is selected from the group consisting of:

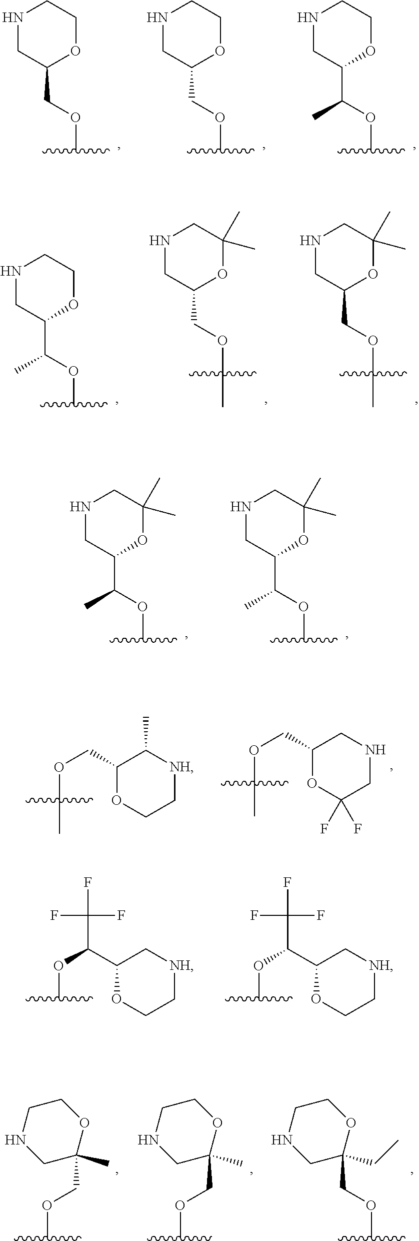

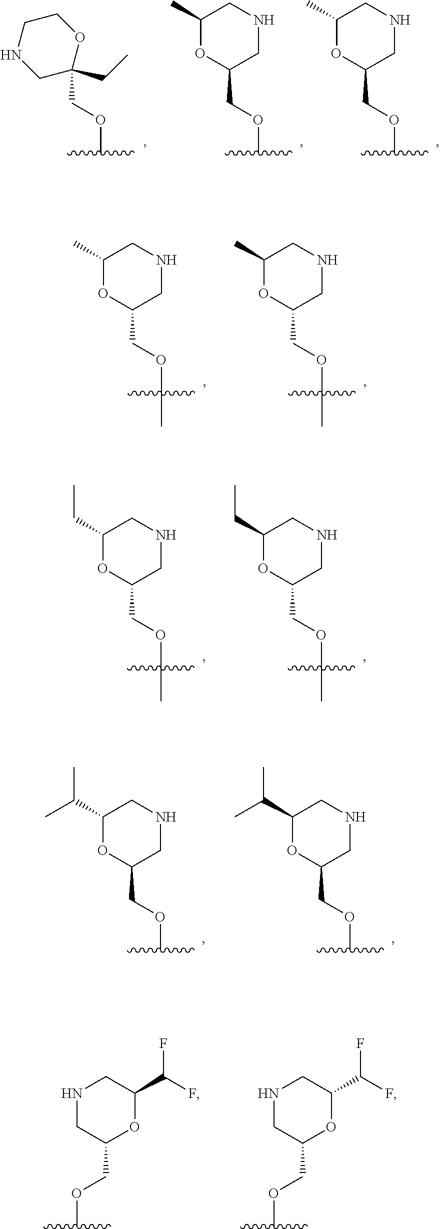

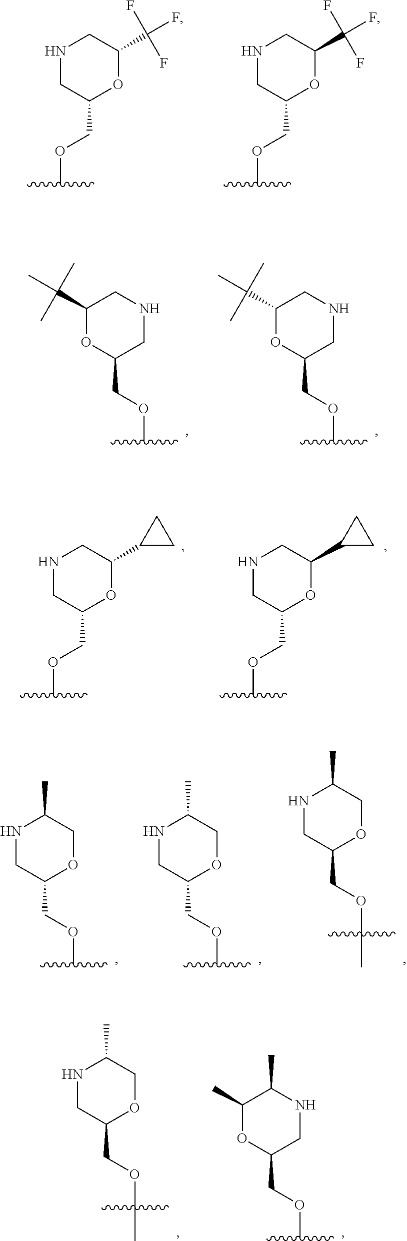

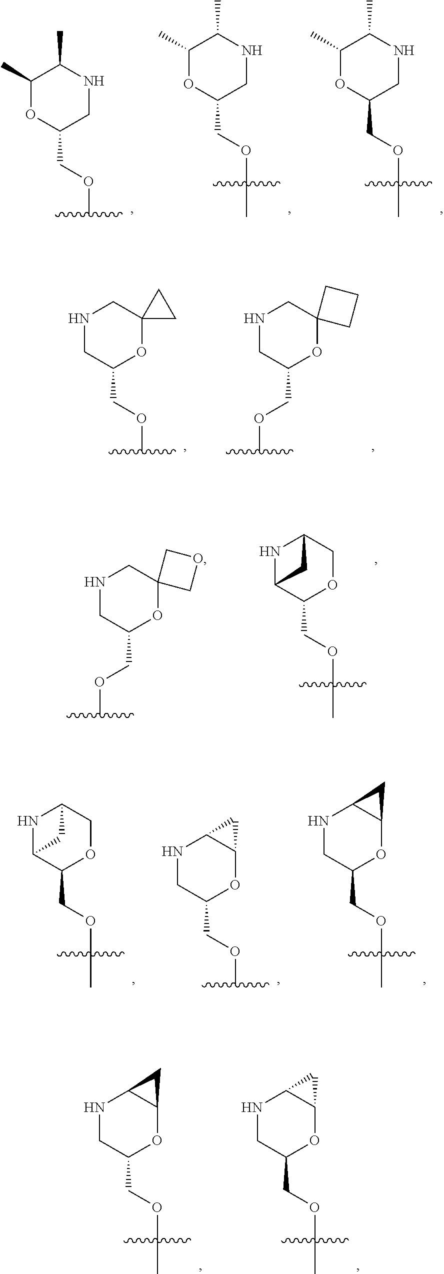

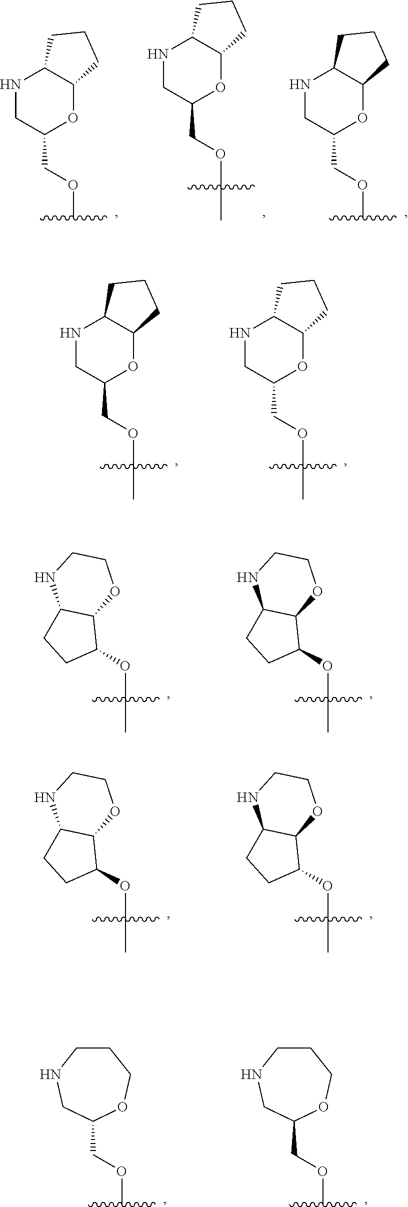

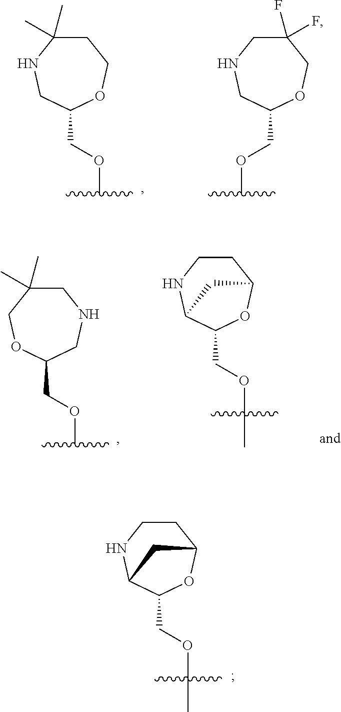 [SMILES: *OC[C@@H]1CNC(C)(C)CCO1 ; *OC[C@@H]1CNCC(F)(F)CO1 ; *OC[C@@H]1CNCC(C)(C)CO1 ; *OC[C@H]1O[C@@H]2CCN[C@H]1C2 ; *OC[C@H]1O[C@H]2CCN[C@@H]1C2]

and especially selected from the group consisting of:

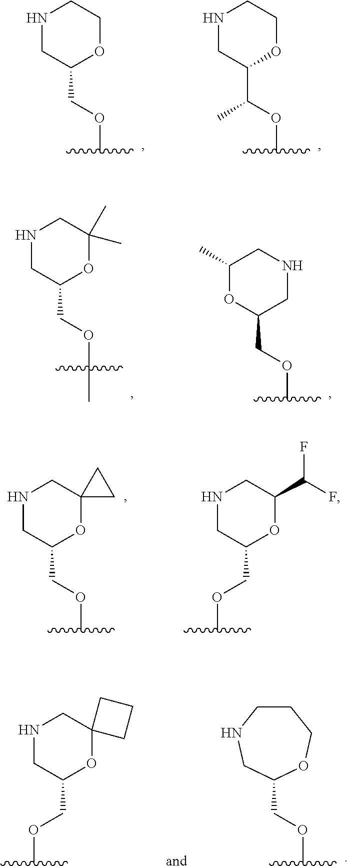

In one embodiment, the compound of Formula (I) is selected from the group consisting of a compound in one of tables 2-4, 6-8 and 10-15.

In another embodiment, the compound of Formula (I) is selected from the group consisting of one of the following compounds, or a pharmaceutically acceptable salt thereof:

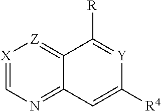

| No | X | Y | Z | R | R⁴ |
|---|---|---|---|---|---|
| 2.44 | CH | N | CH | 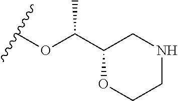 [SMILES: *O[C@H](C)[C@@H]1CNCCO1] | 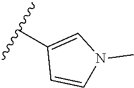 [SMILES: *c1ccn(C)c1] |
| 2.51 | CH | N | CH | 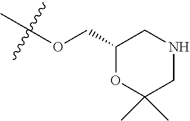 [SMILES: *OC[C@@H]1CNCC(C)(C)O1] | 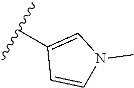 [SMILES: *c1ccn(C)c1] |
| 2.20 | CH | N | CH | 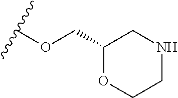 [SMILES: *OC[C@@H]1CNCCO1] | 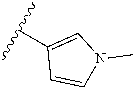 [SMILES: *c1ccn(C)c1] |
| 3.10 | CH | CH | CH | 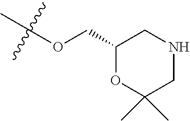 [SMILES: *OC[C@@H]1CNCC(C)(C)O1] | 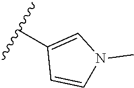 [SMILES: *c1ccn(C)c1] |
| 6.28 | CH | N | CH | 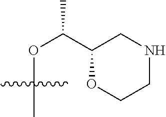 [SMILES: *O[C@H](C)[C@@H]1CNCCO1] | 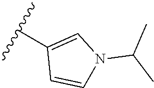 [SMILES: *c1ccn(C(C)C)c1] |
| 6.26 | CH | N | CH | 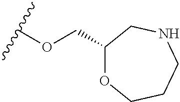 [SMILES: *OC[C@@H]1CNCCCO1] | 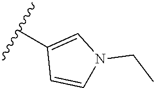 [SMILES: *c1ccn(CC)c1] |
| 3.1 | CH | CH | CH | 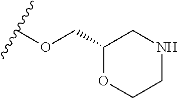 [SMILES: *OC[C@@H]1CNCCO1] | 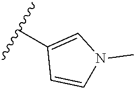 [SMILES: *c1ccn(C)c1] |
| 2.42 | CH | N | CH | 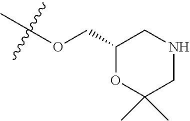 [SMILES: *OC[C@@H]1CNCC(C)(C)O1] | 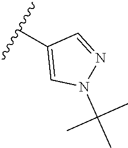 [SMILES: *c1cnn(C(C)(C)C)c1] |
| 6.9 | CH | N | CH | 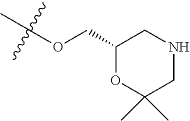 [SMILES: *OC[C@@H]1CNCC(C)(C)O1] | 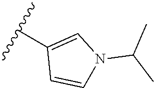 [SMILES: *c1ccn(C(C)C)c1] |
| 6.19 | CH | N | CH | 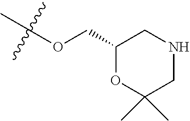 [SMILES: *OC[C@@H]1CNCC(C)(C)O1] | 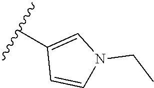 [SMILES: *c1ccn(CC)c1] |
| 2.67 | CH | N | CH | 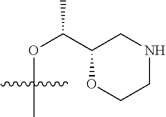 [SMILES: *O[C@H](C)[C@@H]1CNCCO1] | 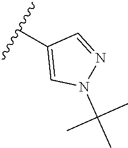 [SMILES: *c1cnn(C(C)(C)C)c1] |
| 6.2 | CH | N | CH | 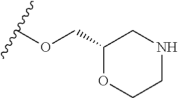 [SMILES: *OC[C@@H]1CNCCO1] | 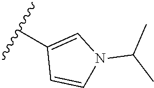 [SMILES: *c1ccn(C(C)C)c1] |
| 14.6 | CH | N | C—CH₃ | 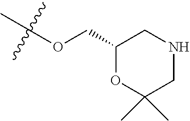 [SMILES: *OC[C@@H]1CNCC(C)(C)O1] | 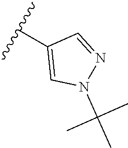 [SMILES: *c1cnn(C(C)(C)C)c1] |
| 3.6 | CH | CH | CH | 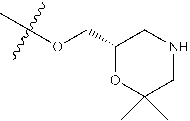 [SMILES: *OC[C@@H]1CNCC(C)(C)O1] | 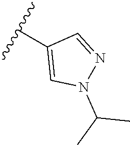 [SMILES: *c1cnn(C(C)C)c1] |
| 7.11 | CH | CH | CH | 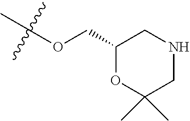 [SMILES: *OC[C@@H]1CNCC(C)(C)O1] | 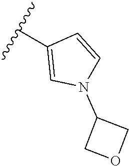 [SMILES: *c1ccn(C2COC2)c1] |
| 2.55 | CH | N | CH | 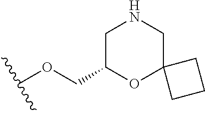 [SMILES: *OC[C@@H]1CNCC2(CCC2)O1] | 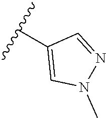 [SMILES: *c1cnn(C)c1] |
| 2.66 | CH | N | CH | 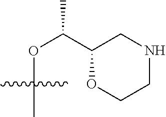 [SMILES: *O[C@H](C)[C@@H]1CNCCO1] | 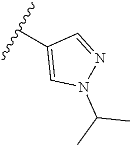 [SMILES: *c1cnn(C(C)C)c1] |
| 12.8 | CH | CH | N | 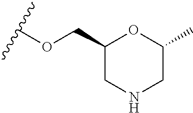 [SMILES: *OC[C@@H]1CNC[C@@H](C)O1] | 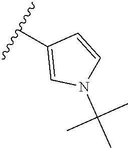 [SMILES: *c1ccn(C(C)(C)C)c1] |
| 4 | CH | CH | CH | 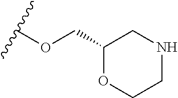 [SMILES: *OC[C@@H]1CNCCO1] | 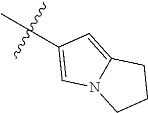 [SMILES: *c1cc2n(c1)CCC2] |
| 14.2 | CH | CH | CH | 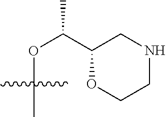 [SMILES: *O[C@H](C)[C@@H]1CNCCO1] | 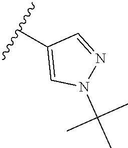 [SMILES: *c1cnn(C(C)(C)C)c1] |
| 1 | CH | CH | CH | 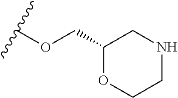 [SMILES: *OC[C@@H]1CNCCO1] | 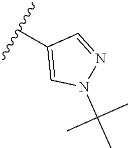 [SMILES: *c1cnn(C(C)(C)C)c1] |
| 6.22 | CH | N | CH | 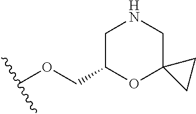 [SMILES: *OC[C@@H]1CNCC2(CC2)O1] | 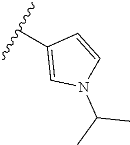 [SMILES: *c1ccn(C(C)C)c1] |
| 2.99 | CH | N | CH | 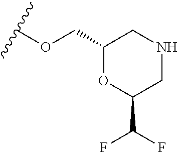 [SMILES: *OC[C@@H]1CNC[C@@H](C(F)F)O1] | 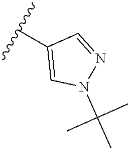 [SMILES: *c1cnn(C(C)(C)C)c1] |

In another embodiment, the compound of Formula (I) is selected from the group consisting of one of the following compounds, or a pharmaceutically acceptable salt thereof:
7-(1-methyl-1H-pyrrol-3-yl)-5-[(1R)-1-[(2S)-morpholin-2-yl]ethoxy]-1,6-naphthyridine
5-{[(2S)-6,6-dimethylmorpholin-2-yl]methoxy}-7-(1-methyl-1H-pyrrol-3-yl)-1,6-naphthyridine
7-(1-methyl-1H-pyrrol-3-yl)-5-{[(2S)-morpholin-2-yl]methoxy}-1,6-naphthyridine
5-{[(2S)-6,6-dimethylmorpholin-2-yl]methoxy}-7-(1-methyl-1H-pyrrol-3-yl)quinoline
5-[(1R)-1-[(2S)-morpholin-2-yl]ethoxy]-7-[1-(propan-2-yl)-1H-pyrrol-3-yl]-1,6-naphthyridine
7-(1-ethyl-1H-pyrrol-3-yl)-5-{[(2S)-1,4-oxazepan-2-yl]methoxy}-1,6-naphthyridine
7-(1-methyl-1H-pyrrol-3-yl)-5{-[(2S)-morpholin-2-yl]methoxy}quinoline
7-(1-tert-butyl-1-pyrazol-4-yl)-5-{[(2S)-6,6-dimethylmorpholin-2-yl]methoxy}-1,6-naphthyridine
5-{[(2S)-6,6-dimethylmorpholin-2-yl]methoxy}-7-[1-(propan-2-yl)-1H-pyrrol-3-yl]-1,6-naphthyridine
5-{[(2S)-6,6-dimethylmorpholin-2-yl]methoxy}-7-(1-ethyl-1H-pyrrol-3-yl)-1,6-naphthyridine
7-(1-tert-butyl-1H-pyrazol-4-yl)-5-[(1R)-1-[(2S)-morpholin-2-yl]ethoxy]-1,6-naphthyridine
5-{[(2S)-morpholin-2-yl]methoxy}-7-[1-(propan-2-yl)-1H-pyrrol-3-yl]-1,6-naphthyridine
7-(1-tert-butyl-1H-pyrazol-4-yl)-5-[(6,6-dimethylmorpholin-2-yl)methoxy]-4-methyl-1,6-naphthyridine
5-{[(2S)-6,6-dimethylmorpholin-2-yl]methoxy}-7-[1-(propan-2-yl)-1H-pyrazol-4-yl]quinoline
5-{[(2S)-6,6-dimethylmorpholin-2-yl]methoxy}-7-[1-(oxetan-3-yl)-1H-pyrrol-3-yl]quinoline
7-(1-methyl-1H-pyrazol-4-yl)-5-{[(6S)-5-oxa-8-azaspiro[3.5]nonan-6-yl]methoxy}-1,6-naphthyridine
5-[(1R)-1-[(2S)-morpholin-2-yl]ethoxy]-7-[1-(propan-2-yl)-1H-pyrazol-4-yl]-1,6-naphthyridine
7-(1-tert-butyl-1H-pyrrol-3-yl)-5-{[(2S,6R)-6-methylmorpholin-2-yl]methoxy}quinoxaline
7-(2,3-dihydro-1H-pyrrolizin-6-yl)-5-{[(2S)-morpholin-2-yl]methoxy}quinoline
7-(1-tert-butyl-1H-pyrazol-4-yl)-5-[(1R)-1-[(2S)-morpholin-2-yl]ethoxy]quinoline
7-(1-tert-butyl-1H-pyrazol-4-yl)-5-{[(2S)-morpholin-2-yl]methoxy}quinoline
5-{[(5S)-4-oxa-7-azaspiro[2.5]octan-5-yl]methoxy}-7-[1-(propan-2-yl)-1-H-pyrrol-3-yl]-1,6-naphthyridine
7-(1-tert-butyl-1-pyrazol-4-yl)-5-{[(2S,6S)-6-(difluoromethyl)morpholin-2-yl]methoxy}-1,6-naphthyridine.

The compound names in the preceding paragraph were derived using ChemAxon Instant JChem 19.8.0.

In one embodiment, the compound of the first aspect, or pharmaceutically acceptable salt or prodrug thereof, is an inhibitor of spleen tyrosine kinase (Syk).

The compound of the first aspect, or a pharmaceutically acceptable salt or prodrug thereof, may have an EC₅₀ for Syk that is less than 500 nM, especially less than 250 nM, more especially less than 100 nM, most especially less than 50 nM.

In one embodiment, the compound of the first aspect, or a pharmaceutically acceptable salt or prodrug thereof, may have a permeability of PₐₚₚA-B of more than 10×10⁻⁶ cm/s, especially more than 15×10⁻⁶ cm/s, most especially more than 20×10⁶ cm/s. In another embodiment, the compound of the first aspect, or a pharmaceutically acceptable salt or prodrug thereof, may have an efflux ratio of less than 2.0, especially less than 1.5 and most especially less than 1.0.

The compound of the first aspect, or a pharmaceutically acceptable salt or prodrug thereof, may have a K_{pu,u} of more than 0.05, especially more than 0.1, more especially more than 0.2, most especially more than 0.3. The compound of the first aspect, or a pharmaceutically acceptable salt or prodrug thereof, post an oral dose of 10 mg/kg may have free brain levels of greater than 10 nM, especially more than 25 nM, or greater than 50 nM.

As used herein, terminology such as

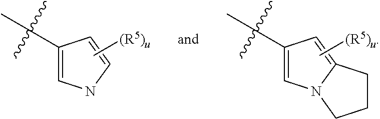

and means that u R⁵ substituents may be appended to the cyclic system, and at any position, including where appropriate on a nitrogen atom (such as the pyrrole N) or on either ring (for example in the 2,3-dihydro pyrrolizine, R⁵ may be appended on the pyrrole portion, or on the pyrrolidine portion). In

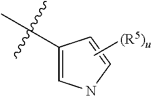

for example, if the pyrrole N is not substituted by R⁵ then the pyrrole N is NH.

Unless defined otherwise, all technical and scientific terms used herein have the same meaning as would be commonly understood by those of ordinary skill in the art to which this invention belongs.

Reference throughout this specification to ‘one embodiment’ or ‘an embodiment’ means that a particular feature, structure, or characteristic described in connection with the embodiment is included in at least one embodiment of the present invention. Thus, the appearance of the phrases ‘in one embodiment’ or ‘in an embodiment’ in various places throughout this specification are not necessarily all referring to the same embodiment. Furthermore, the particular features, structures, or characteristics may be combined in any suitable manner in one or more combinations.

The term “alkyl” refers to a straight-chain or branched alkyl substituent containing from, for example, 1 to about 12 carbon atoms, preferably 1 to about 8 carbon atoms, more preferably 1 to about 6 carbon atoms, even more preferably from 1 to about 4 carbon atoms. Examples of suitable alkyl groups include, but are not limited to, methyl, ethyl, propyl, isopropyl, n-butyl, sec-butyl, isobutyl, tert-butyl, pentyl, isoamyl, 2-methylbutyl, 3-methylbutyl, hexyl, heptyl, 2-methylpentyl, 3-methylpentyl, 4-methylpentyl, 2-ethylbutyl, 3-ethylbutyl, octyl, nonyl, decyl, undecyl, dodecyl and the like. The number of carbons referred to relates to the carbon backbone and carbon branching but does not include carbon atoms belonging to any substituents, for example the carbon atoms of an alkoxy substituent branching off the main carbon chain.

The term “fluoroalkyl”, “cyclofluoroalkyl”, “fluoroalkenyl”, “fluoroalkynyl”, “fluoroheterocyclyl” and the like refers to an alkyl, cycloalkyl, alkenyl, alkynyl or heterocyclyl group in which one or more of the hydrogen atoms have been replaced with fluorine. In one embodiment, less than 10%, 20%, 30%, 40%, 50%, 60%, 70%, 80% or 90% of the hydrogen atoms in the relevant group have been replaced with fluorine. In another embodiment, more than 10%, 20%, 30%, 40%, 50%, 60%, 70%, 80% or 90% of the hydrogen atoms in the relevant group have been replaced with fluorine. A fluoroalkyl group may include, for example, only one fluorine atom, or may be a perfluoroalkyl group. For example, a cyclofluoroalkyl group may be a 3 to 8 membered cyclofluoroalkyl ring; especially a 3 to 7 membered cyclofluoroalkyl ring.

The term “alkenyl” refers to a straight-chain or branched alkenyl substituent containing from, for example, 2 to about 12 carbon atoms, preferably 2 to about 8 carbon atoms, more preferably 2 to about 6 carbon atoms. Examples of suitable alkenyl groups include, but are not limited to, ethenyl, propenyl, isopropenyl, butenyl, butadienyl, pentenyl, pentadienyl, hexenyl, hexadienyl, heptenyl, octenyl, nonenyl, decenyl, undecenyl, dodecenyl and the like. Branched alkenyl groups may be branched at any suitable position, and exemplary branched alkenyl groups may include, for example, 2-methyl-1-pentenyl, 3-methyl-1-pentenyl, 2-methyl-2-pentenyl, 2-methyl-3-pentenyl, 2-methyl-4-pentenyl and the like. The number of carbons referred to relates to the carbon backbone and carbon branching but does not include carbon atoms belonging to any substituents, for example the carbon atoms of an alkoxy substituent branching off the main carbon chain.

The term “alkynyl” refers to a straight-chain or branched alkynyl substituent containing from, for example, 2 to about 12 carbon atoms, preferably 2 to about 8 carbon atoms, more preferably 2 to about 6 carbon atoms. Examples of suitable alkynyl groups include, but are not limited to, ethynyl, propynyl (such as prop-2-ynyl or prop-1-ynyl), butynyl, butadiynyl, pentynyl, hexynyl, heptynyl, octynyl, nonynyl, decynyl, undecynyl, dodecynyl and the like. Branched alkynyl groups may be branched at any suitable position, and exemplary branched alkynyl groups may include, for example, 3-methyl-1-pentynyl, 2-methyl-3-pentynyl, 2-methyl-4-pentynyl and the like. The number of carbons referred to relates to the carbon backbone and carbon branching but does not include carbon atoms belonging to any substituents, for example the carbon atoms of an alkoxy substituent branching off the main carbon chain.

The term “cycloalkyl” refers to a saturated non-aromatic cyclic hydrocarbon. The cycloalkyl ring may include a specified number of carbon atoms. For example, a 3 to 8 membered cycloalkyl group includes 3, 4, 5, 6, 7 or 8 carbon atoms. The cycloalkyl group may be monocyclic, bicyclic or tricyclic. When more than one ring is present the rings are fused together (for example, a bicyclic ring is fused if two atoms are common to both rings) or linked by a common atom (for example, a spiro compound). Non-limiting examples may include cyclopropyl, cyclobutyl, cyclopentyl, cyclohexyl and the like. A cycloalkyl group may be, for example, a 3 to 8 membered cycloalkyl ring; especially a 3 to 7 membered cycloalkyl ring.

The term “cycloalkenyl” or “cycloalkene” refers to a cyclic hydrocarbon having at least one double bond, which is not aromatic. The cycloalkenyl ring may include a specified number of carbon atoms. For example, a 5 membered cycloalkenyl group includes 5 carbon atoms. The cycloalkenyl group may be monocyclic, bicyclic or tricyclic. When more than one ring is present the rings are fused together (for example, a bicyclic ring is fused if two atoms are common to both rings) or linked by a common atom (for example, a spiro compound). Non-limiting examples may include cyclopentenyl and cyclopenta-1,3-dienyl.

The term “aryl” or “aromatic” refers to an aromatic carbocyclic substituent, as commonly understood in the art. It is understood that the term aryl applies to cyclic substituents in which at least one ring is planar and comprises 4n+2 π electrons, according to Hückel's Rule. Aryl groups may be monocyclic, bicyclic or tricyclic. Examples of aryl groups include, but are not limited to, phenyl and naphthyl. Aryl groups do not encompass cycloalkyl groups, and aryl groups have a ring system (for example monocyclic, bicyclic or tricyclic rings) in which at least one ring is aromatic. For example, both naphthyl and 1,2,3,4-tetrahydronaphthyl groups would be aryl or aromatic groups. When more than one ring is present the rings are fused together (for example, a bicyclic ring is fused if two atoms are common to both rings) or linked by a common atom (for example, a spiro compound which may be present in a non-aromatic ring).

The term “heterocyclic” or “heterocyclyl” as used herein, refers to a cycloalkyl or cycloalkenyl group in which one or more carbon atoms have been replaced by heteroatoms independently selected from N, S and O. For example, between 1 and 4 carbon atoms in each ring may be replaced by heteroatoms independently selected from N, S and O. The heterocyclyl group may be monocylic, bicyclic or tricyclic in which at least one ring includes a heteroatom. When more than one ring is present the rings are fused together (for example, a bicyclic ring is fused if two atoms are common to both rings) or linked by a common atom (for example, a spiro compound). Each of the rings of a heterocyclyl group may include, for example, between 5 and 7 atoms. Examples of heterocyclyl groups include tetrahydrofuranyl, tetrahydrothiophenyl, pyrrolidinyl, pyrrolinyl, dithiolyl, 1,3-dioxanyl, dioxinyl, piperidinyl, piperazinyl, morpholinyl, thiomorpholinyl, pyranyl, 1,4-dithianyl, and decahydroisoquinolyl. In a bicyclic or tricyclic heterocyclyl group, none of the rings are aromatic. In one embodiment, heterocyclyl may be optionally substituted by ═O.

The term “heteroaryl” or “heteroaromatic”, as used herein, refers to a monocyclic, bicyclic or tricyclic ring of up to 7 atoms in each ring, wherein at least one ring is aromatic and at least one ring contains from 1 to 4 heteroatoms selected from the group consisting of O, N and S. When more than one ring is present the rings are fused together (for example, a bicyclic ring is fused if two atoms are common to both rings) or linked by a common atom (for example, a spiro compound which may be present in a non-aromatic ring). Consideration must be provided to tautomers of heteroatom containing ring systems containing carbonyl groups, for example, when determining if a ring is a heterocyclyl or heteroaryl ring. Heteroaryl includes, but is not limited to, 5-membered heteroaryls having one hetero atom (e.g., thiophenes, pyrroles, furans); 5 membered heteroaryls having two heteroatoms in 1,2 or 1,3 positions (e.g., oxazoles, pyrazoles, imidazoles, thiazoles); 5-membered heteroaryls having three heteroatoms (e.g., triazoles, thiadiazoles, oxadiazoles, furazanes); 5-membered heteroaryls having four heteroatoms (e.g., tetrazoles); 6-membered heteroaryls with one heteroatom (e.g., pyridine); 6-membered heteroaryls with two heteroatoms (e.g., pyridazines, cinnolines, phthalazines, pyrazines, pyrimidines, quinazolines, quinoxalines); 6-membered heteroaryls with three heteroatoms (e.g., 1,3,5-triazine); and 6-membered heteroaryls with four heteroatoms. Examples of heteroaryl include thiophene, benzothiophene, benzofuran, benzimidazole, benzoxazole, benzothiazole, benzisothiazole, furan, pyrrole, imidazole, pyrazole, triazole, triazine, thiadiazole, oxadiazole, tetrazole, furazane, pyridine, pyrazine, pyrimidine, pyridazine, indole, isoindole, 1H-indazole, purine, quinoline, isoquinoline, phthalazine, naphthyridine, quinoxaline, quinazoline, cinnoline, carbazole, phenanthridine, acridine, phenazine, thiazole, isothiazole, phenothiazine, oxazole, isooxazole, furazane, and phenoxazine. Further exemplary heteroaryl groups may include, for example, indoline or 2,3-dihydrobenzofuran. In one embodiment, heteroaryl may be optionally substituted by ═O.

Whenever a range of the number of atoms in a structure is indicated (e.g., a C₁₋₁₂, C₁₋₆ alkyl, etc.), it is specifically contemplated that any sub-range or individual number of carbon atoms falling within the indicated range also can be used. Thus, for instance, the recitation of a range of 1-12 carbon atoms (e.g., C₁₋₁₂), 1-6 carbon atoms (e.g., C₁₋₆) as used with respect to any chemical group (e.g., alkyl, etc.) referenced herein encompasses and specifically describes 1, 2, 3, 4, 5, 6, 7, 8, 9, 10, 11, and/or 12 carbon atoms, as appropriate, as well as any sub-range thereof (e.g., 1-2 carbon atoms, 1-3 carbon atoms, 1-4 carbon atoms, 1-5 carbon atoms, 1-6 carbon atoms, 1-7 carbon atoms, 1-8 carbon atoms, 1-9 carbon atoms, 1-10 carbon atoms, 1-11 carbon atoms, 1-12 carbon atoms, 2-3 carbon atoms, 2-4 carbon atoms, 2-5 carbon atoms, 2-6 carbon atoms, 2-7 carbon atoms, 2-8 carbon atoms, 2-9 carbon atoms, 2-10 carbon atoms, 2-11 carbon atoms, 2-12 carbon atoms, 3-4 carbon atoms, 3-5 carbon atoms, 3-6 carbon atoms, 3-7 carbon atoms, 3-8 carbon atoms, 3-9 carbon atoms, 3-10 carbon atoms, 3-11 carbon atoms, 3-12 carbon atoms, 4-5 carbon atoms, 4-6 carbon atoms, 4-7 carbon atoms, 4-8 carbon atoms, 4-9 carbon atoms, 4-10 carbon atoms, 4-11 carbon atoms, and/or 4-12 carbon atoms, etc., as appropriate).

As used herein, “halo” refers to a halogen atom, especially F, Cl or Br; more especially F or Cl; most especially F.

As used herein, the term “optionally substituted” means that any number of hydrogen atoms on the optionally substituted group are replaced with another moiety. Exemplary optional substituents are discussed above, for example in R⁵.

The term “pharmaceutically acceptable salt”, as used herein, refers to salts which are toxicologically safe for systemic or localised administration such as salts prepared from pharmaceutically acceptable non-toxic bases or acids including inorganic or organic bases and inorganic or organic acids; especially a salt prepared from a pharmaceutically acceptable inorganic or organic acid.

The term “a 4 to 6-membered oxygen containing heterocyclic ring”, as used herein may include, for example, oxetanyl, tetrahydrofuranyl or pyranyl ring systems. An oxetanyl ring system may be preferred.

The prodrug form of the above compounds may include compounds of Formula (I) derivatised at the nitrogen atom of the morpholine or homomorpholine group. The prodrug form of the above compounds may be also considered to include a C₁-C₂₀ ester or ester comprising a cycloalkyl, or aryl moiety, for example from an OH group in substituent R₄. The aryl moiety may include substituted phenyl or fused 2-3 cyclic aromatic rings. Suitable prodrugs may include those defined in Simplicio, A. L. et al., 2008. Prodrugs for amines. Molecules, 13(3), pp. 519-547 or Safadi, M. et al., 1993. Phosphoryloxymethyl carbamates and carbonates—novel water-soluble prodrugs for amines and hindered alcohols. Pharmaceutical research 10(9), pp. 1350-1355, and may include N-alkyl, amides, carbamates or carbonates (such as phosphoryloxymethyl carbamates and carbonates).

According to a second aspect of the present invention, there is provided a pharmaceutical composition comprising an effective amount of the compound of the first aspect, or a pharmaceutically acceptable salt or prodrug thereof. The composition may further comprise a pharmaceutically acceptable carrier, diluent and/or excipient.

While it is possible that the compound of Formula (I) (or a pharmaceutical salt or prodrug thereof) may be administered as a neat chemical, it also may be administered as part of a pharmaceutical composition which includes at least one carrier or excipient.

The type of pharmaceutical composition may depend upon the Absorption, Distribution, Metabolism and Excretion (ADME) profile of the compound of Formula (I) (or a pharmaceutical salt or prodrug thereof). For example, it may be most appropriate for compounds of Formula (I) (or a pharmaceutical salt or prodrug thereof) to be administered parenterally, especially intravenously, and consequently the pharmaceutical composition may be formulated for parenteral or intravenous administration. However, and preferably, the pharmaceutical composition may include those suitable for oral or rectal administration, or for administration by non-intravenous routes. An oral composition for oral administration may be preferred.

Parenteral administration may include administration by one or more of the following routes: intravenously, intrathecally, cutaneously, subcutaneously, nasally, intramuscularly, intraocularly, transepithelially, vaginally, intraperitoneally and topically. Topical administration includes buccal, sub-lingual, dermal, ocular, rectal, nasal, as well as administration by inhalation or by aerosol means. For intravenous, cutaneous or subcutaneous injection, or injection at a site where treatment is desired, the active agent may be in the form of a parenterally acceptable aqueous solution which is pyrogen-free and has suitable pH, isotonicity and stability. Those of skill in the art would be able to prepare suitable solutions.

The nature of the pharmaceutical composition and the carrier or excipient will depend on the route of administration and the nature of the condition and the patient being treated. It is believed that the choice of a particular carrier, excipient or delivery system, and route of administration could be readily determined by a person skilled in the art. In some circumstances it may be necessary to protect the compound of Formula (I) (or a pharmaceutical salt or prodrug thereof) by means known in the art, for example, by micro encapsulation. The route of administration should also be chosen such that the active agent reaches its site of action. The pharmaceutical composition may include any suitable effective amount of the active agent commensurate with the intended dosage range to be employed.

The pharmaceutical composition may be in the form of a solid (including tablets, filled capsules, powders, cachets, capsules, troches, suppositories, wafers, dispersible granules and pessaries), or a liquid (including solutions, suspensions, syrups, emulsions, colloids, elixirs, creams, gels and foams). In one embodiment, the pharmaceutical composition may be in the form of a sterile injectable solution for parenteral use.

The pharmaceutically acceptable carrier(s) or excipient(s) must be acceptable in the sense of being compatible with the other components in the composition and not being deleterious to the patient. The pharmaceutically acceptable carrier or excipient may be either a solid or a liquid. The carrier or excipient may act as a diluent, buffer, stabiliser, isotonicising agent, flavouring agent, anti-oxidant, solubilizer, lubricant, suspending agent, binder, preservative, tablet disintegrating agent or an encapsulating material. Suitable carriers and excipients would be known to a skilled person. With regard to buffers, aqueous compositions may include buffers for maintaining the composition at close to physiological pH1 or at least within a range of about pH 6.0 to 9.0.

If the pharmaceutical composition is a powder, the active agent (the compound of Formula (I) or a pharmaceutically acceptable salt thereof) and a carrier or excipient may both be finely divided powders which are mixed together, for example using processes known in the art such as dry blending or wet granulation.

If the pharmaceutical composition is a tablet, the active agent may be mixed with a suitable amount of a carrier or excipient which has the necessary binding capacity before compaction into a tablet of the desired shape and size.

Powders or tablets may include any suitable amount of the active agent, and exemplary amounts of the active agent in the powder or tablet may range from about five or ten percent to about seventy percent. Exemplary carriers or excipients for powders and tablets may include, for example, magnesium carbonate, talc, sugar, lactose, pectin, dextrin, starch, gelatin, tragacanth, methylcellulose, a low melting wax, cocoa butter and the like.

Liquid form preparations may include, for example, water, saline, water-dextrose, water-propylene glycol, petroleum, or oil (including animal, vegetable mineral or synthetic oil) solutions. For example, parenteral injection liquid preparations may be formulated as solutions in aqueous polyethylene glycol solution. Such liquid form preparations may contain at least 0.1 wt % of the active compound.

Liquid pharmaceutical compositions may be formulated in unit dose form. For example, the compositions may be presented in ampoules, pre-filled syringes, small volume infusions or in multi-dose containers. Such compositions may include a preservative. The compositions may also include formulatory agents such as suspending, stabilising and/or dispersing agents. The composition may also be in powder form for constitution with a suitable vehicle (such as sterile water) before use. Liquid carriers and excipients may include colorants, flavours, stabilizers, buffers, artificial and natural sweeteners, dispersants, thickeners, solubilizing agents, suspending agents and the like.

Aqueous solutions for oral use may be prepared by dissolving the active agent in water and adding colourants, thickeners, flavours, and stabilizing agents, as necessary. Aqueous suspensions for oral use may be prepared by dispersing the active agent in water with viscous material, such as natural or synthetic gums, resins, methyl cellulose or other suspending agents.

For topical administration to the epidermis the compounds may be formulated as an ointment, cream or lotion, or as a transdermal patch.

The compositions may also be administered by inhalation in the form of an aerosol spray from a pressurised dispenser or container, which contains a propellant such as carbon dioxide gas, a hydrofluoroalkane, nitrogen, propane or other suitable gas or gas combination. The pharmaceutical composition may be in a form suitable for administration by inhalation or insufflation.

The pharmaceutical composition may be adapted to provide sustained release of the active agent.

The pharmaceutical composition may be in unit dosage form. In such form, the pharmaceutical composition may be prepared as unit doses containing appropriate quantities of the active agent. The unit dosage form may be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

According to a third aspect of the present invention, there is provided a method of treating or preventing a disease, disorder or condition associated with spleen tyrosine kinase activity in a subject, the method comprising administering to the subject an effective amount of the compound of the first aspect or a pharmaceutically acceptable salt or prodrug thereof, or the pharmaceutical composition of the second aspect. The disease, disorder or condition associated with spleen tyrosine kinase activity may affect or be in the Central Nervous System. In another embodiment, the disease, disorder or condition associated with spleen tyrosine kinase activity may affect or be in the Peripheral Nervous System The disease, disorder or condition associated with spleen tyrosine kinase activity may be associated with the brain of a subject. The disease, disorder or condition associated with spleen tyrosine kinase activity may be associated with a region of the subject outside of the brain.

According to a fourth aspect of the present invention, there is provided a method of treating or preventing one or more of: glioblastoma, cancer (especially ovarian cancer, head and neck cancer, eye cancer (retinoblastoma), leukaemia (especially B-cell and T-cell lymphoma), lymphoma (including Waldenstroem's macroglobulinemia), bone cancer, liver cancer, lung cancer (especially small cell lung cancer), blood cancer (including macroglobulinemia)), osteoporosis, rheumatoid arthritis, liver disease (including liver fibrosis, viral hepatitis, alcoholic liver disease, non-alcoholic steatohepatitis and hepatocellular carcinoma), fibrosis (especially peritoneal fibrosis), periodontal diseases (for example diseases associated with alveolar bone resorption), diabetes (especially Type I diabetes), inflammation (especially dermatitis, fasciitis or pulmonary inflammation), Graves' disease, lung diseases or disorders (including hantavirus pulmonary syndrome), kidney disease (including glomerulonephritis), epidermolysis bullosa acquisita, Wiskott-Aldrich syndrome, agammaglobulinemia, Nasu-Hakola disease, allergy (including pharmaceutical allergy, especially co-trimoxazole allergy and carbapenem allergy), microbial infection (especially bacterial infection, more especially Mycobacterium abscessus), fungal infection (including Chromoblastomycosis, and mycosis), autoimmune hypersensitivity disease, bleeding disorders, thrombocytopenia, bone or skeletal disorders (including Melnick-Needles syndrome and otopalatodigital syndrome spectrum disorder), nail disease, chronic mucocutaneous candidiasis, a neurological disease or disorder (including Alzheimer's disease, dementia, multiple sclerosis and Parkinson's disease), a neuroinflammatory disease, stroke, traumatic brain injury, and subarachnoid haemorrhage; the method comprising administering to the subject an effective amount of the compound of the first aspect or a pharmaceutically acceptable salt or prodrug thereof, or the pharmaceutical composition of the second aspect.

According to a fifth aspect of the present invention, there is provided a use of the compound of the first aspect, or a pharmaceutically acceptable salt or prodrug thereof, in the manufacture of a medicament for the treatment or prevention of a disease, disorder or condition associated with spleen tyrosine kinase activity in a subject. The disease, disorder or condition associated with spleen tyrosine kinase activity may affect or be in the Central Nervous System. In another embodiment, the disease, disorder or condition associated with spleen tyrosine kinase activity may affect or be in the Peripheral Nervous System. The disease, disorder or condition associated with spleen tyrosine kinase activity may be associated with the brain of a subject. The disease, disorder or condition associated with spleen tyrosine kinase activity may be associated with a region of the subject outside of the brain.

According to a sixth aspect of the present invention, there is provided a use of the compound of the first aspect, or a pharmaceutically acceptable salt or prodrug thereof, in the manufacture of a medicament for the treatment or prevention of one or more of: glioblastoma, cancer (especially ovarian cancer, head and neck cancer, eye cancer (retinoblastoma), leukaemia (especially B-cell and T-cell lymphoma), lymphoma (including Waldenstroem's macroglobulinemia), bone cancer, liver cancer, lung cancer (especially small cell lung cancer), blood cancer (including macroglobulinemia)), osteoporosis, rheumatoid arthritis, liver disease (including liver fibrosis, viral hepatitis, alcoholic liver disease, non-alcoholic steatohepatitis and hepatocellular carcinoma), fibrosis (especially peritoneal fibrosis), periodontal diseases (for example diseases associated with alveolar bone resorption), diabetes (especially Type I diabetes), inflammation (especially dermatitis, fasciitis or pulmonary inflammation), Graves' disease, lung diseases or disorders (including hantavirus pulmonary syndrome), kidney disease (including glomerulonephritis), epidermolysis bullosa acquisita, Wiskott-Aldrich syndrome, agammaglobulinemia, Nasu-Hakola disease, allergy (including pharmaceutical allergy, especially co-trimoxazole allergy and carbapenem allergy), microbial infection (especially bacterial infection, more especially Mycobacterium abscessus), fungal infection (including Chromoblastomycosis, and mycosis), autoimmune hypersensitivity disease, bleeding disorders, thrombocytopenia, bone or skeletal disorders (including Melnick-Needles syndrome and otopalatodigital syndrome spectrum disorder), nail disease, chronic mucocutaneous candidiasis, a neurological disease or disorder (including Alzheimer's disease, dementia and Parkinson's disease), a neuroinflammatory disease, stroke, traumatic brain injury, and subarachnoid haemorrhage in a subject.

According to a seventh aspect of the present invention, there is provided the compound of the first aspect or a pharmaceutically acceptable salt or prodrug thereof, or the pharmaceutical composition of the second aspect, for use in the treatment or prevention of a disease, disorder or condition associated with spleen tyrosine kinase activity. The disease, disorder or condition associated with spleen tyrosine kinase activity may affect or be in the Central Nervous System. In another embodiment, the disease, disorder or condition associated with spleen tyrosine kinase activity may affect or be in the Peripheral Nervous System The disease, disorder or condition associated with spleen tyrosine kinase activity may be associated with the brain of a subject. The disease, disorder or condition associated with spleen tyrosine kinase activity may be associated with a region of the subject outside of the brain.

According to an eighth aspect of the present invention, there is provided the compound of the first aspect or a pharmaceutically acceptable salt or prodrug thereof, or the pharmaceutical composition of the second aspect, for use in the treatment or prevention of one or more of glioblastoma, cancer (especially ovarian cancer, head and neck cancer, eye cancer (retinoblastoma), leukaemia (especially B-cell and T-cell lymphoma), lymphoma (including Waldenstroem's macroglobulinemia), bone cancer, liver cancer, lung cancer (especially small cell lung cancer), blood cancer (including macroglobulinemia)), osteoporosis, rheumatoid arthritis, liver disease (including liver fibrosis, viral hepatitis, alcoholic liver disease, non-alcoholic steatohepatitis and hepatocellular carcinoma), fibrosis (especially peritoneal fibrosis), periodontal diseases (for example diseases associated with alveolar bone resorption), diabetes (especially Type I diabetes), inflammation (especially dermatitis, fasciitis or pulmonary inflammation), Graves' disease, lung diseases or disorders (including hantavirus pulmonary syndrome), kidney disease (including glomerulonephritis), epidermolysis bullosa acquisita, Wiskott-Aldrich syndrome, agammaglobulinemia, Nasu-Hakola disease, allergy (including pharmaceutical allergy, especially co-trimoxazole allergy and carbapenem allergy), microbial infection (especially bacterial infection, more especially Mycobacterium abscessus), fungal infection (including Chromoblastomycosis, and mycosis), autoimmune hypersensitivity disease, bleeding disorders, thrombocytopenia, bone or skeletal disorders (including Melnick-Needles syndrome and otopalatodigital syndrome spectrum disorder), nail disease, chronic mucocutaneous candidiasis, a neurological disease or disorder (including Alzheimer's disease, dementia and Parkinson's disease), a neuroinflammatory disease, stroke, traumatic brain injury, and subarachnoid haemorrhage.

The disease, disorder or condition associated with spleen tyrosine kinase activity may be selected from one or more of the group consisting of: glioblastoma, cancer (especially ovarian cancer, head and neck cancer, eye cancer (retinoblastoma), leukaemia (especially B-cell and T-cell lymphoma), lymphoma (including Waldenstroem's macroglobulinemia), bone cancer, liver cancer, lung cancer (especially small cell lung cancer), blood cancer (including macroglobulinemia)), osteoporosis, rheumatoid arthritis, liver disease (including liver fibrosis, viral hepatitis, alcoholic liver disease, non-alcoholic steatohepatitis and hepatocellular carcinoma), fibrosis (especially peritoneal fibrosis), periodontal diseases (for example diseases associated with alveolar bone resorption), diabetes (especially Type I diabetes), inflammation (especially dermatitis, fasciitis or pulmonary inflammation), Graves' disease, lung diseases or disorders (including hantavirus pulmonary syndrome), kidney disease (including glomerulonephritis), epidermolysis bullosa acquisita, Wiskott-Aldrich syndrome, agammaglobulinemia, Nasu-Hakola disease, allergy (including pharmaceutical allergy, especially co-trimoxazole allergy and carbapenem allergy), microbial infection (especially bacterial infection, more especially Mycobacterium abscessus), fungal infection (including Chromoblastomycosis, and mycosis), autoimmune hypersensitivity disease, bleeding disorders, thrombocytopenia, bone or skeletal disorders (including Melnick-Needles syndrome and otopalatodigital syndrome spectrum disorder), nail disease, chronic mucocutaneous candidiasis, a neurological disease or disorder (including Alzheimer's disease, dementia and Parkinson's disease), a neuroinflammatory disease, stroke, traumatic brain injury, and subarachnoid haemorrhage. The disease, disorder or condition associated with spleen tyrosine kinase activity may affect or be in the Central Nervous System.

In the present specification and claims, the word ‘comprising’ and its derivatives including ‘comprises’ and ‘comprise’ include each of the stated integers but does not exclude the inclusion of one or more further integers.

As used herein, the terms “treatment” (or “treating”) and “prevention” (or “preventing”) are to be considered in their broadest contexts. For example, the term “treatment” does not necessarily imply that a patient is treated until full recovery. The term “treatment” includes amelioration of the symptoms of a disease, disorder or condition, or reducing the severity of a disease, disorder or condition. Similarly, “prevention” does not necessarily imply that a subject will never contract a disease, disorder or condition. “Prevention” may be considered as reducing the likelihood of onset of a disease, disorder or condition, or preventing or otherwise reducing the risk of developing a disease, disorder or condition.

As used herein, the terms “subject” or “individual” or “patient” may refer to any subject, particularly a vertebrate subject, and even more particularly a mammalian subject, for whom therapy is desired. Suitable vertebrate animals include, but are not restricted to, primates, avians, livestock animals (e.g., sheep, cows, horses, donkeys, pigs), laboratory test animals (e.g., rabbits, mice, rats, guinea pigs, hamsters), companion animals (e.g., cats, dogs) and captive wild animals (e.g., foxes, deer, dingoes) A preferred subject is a human.

As used herein, “effective amount” refers to the administration of an amount of the relevant active agent sufficient to at least partially attain the desired response, or to prevent the occurrence of symptoms of the disease, disorder or condition being treated, or to bring about a halt in the worsening of symptoms or to treat and alleviate or at least reduce the severity of the symptoms. The amount may vary depending on factors such as: the health and physical condition of the individual to whom the compound is administered, the taxonomic group of the individual to whom the compound is administered, the extent of treatment/prevention desired, the formulation of the composition, and the assessment of the medical situation. It is expected that the “effective amount” will fall within a broad range that can be determined through routine trials. An effective amount in relation to a human patient, for example, may lie in the range of about 0.1 ng per kg of body weight to 1 g per kg of body weight per dosage, or in the range of about 100 ng to 100 mg per kg of body weight per dosage. Dosage regimes may be adjusted to provide the optimum therapeutic response. For example, several doses may be administered daily, bi-weekly or weekly, or at other suitable time intervals, or the dose may be proportionally reduced as indicated by the circumstances. Decisions on dosage and the like would be within the skill of the medical practitioner or veterinarian responsible for the care of the patient.

The compound of Formula (I) (or a pharmaceutically acceptable salt or prodrug thereof) may be administered with a further active agent. For example, if the disease, disorder or condition being treated or prevented is cancer, then the compound of Formula (I) can be administered with other cancer drugs (such as docetaxel, 5-fluorouracil and the like).

In a ninth aspect, the present invention relates to a method of synthesizing a compound of Formula (I) of the first aspect, the method comprising the steps of:
(a) Coupling

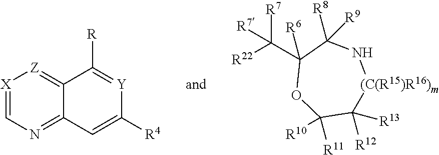

to provide

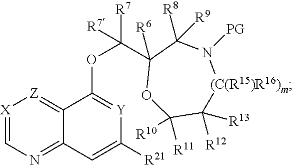

(b) Coupling

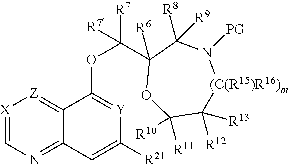

and R²³—R⁴ in the presence of a catalyst to provide

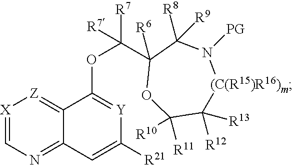

(c) Removing PG from

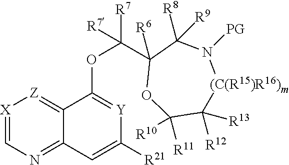

to provide the compound of Formula (I);
wherein:
X, Y, Z, R⁴, R⁶, R⁷, R^{7′}, R⁸, R⁹, R¹⁰, R¹¹, R¹², R¹³, R¹⁵ and R¹⁶ are as defined in the first aspect;
one of R²⁰ and R²² is OH and the other is a leaving group;
PG is a protecting group; and:
   R²¹ is a leaving group; and R²³ is a group that provides an activated carbon at the carbon to which R²³ is attached in R⁴; or
   R²³ is a leaving group; and R²¹ is a group that provides an activated carbon at the carbon to which R²¹ is attached.

As used herein, the term “leaving group” may refer to, for example, halo (such as F, Cl, Br or I) or an activated oxygen group (such as a sulfonyloxy group, including a toluenesulfonyloxy group, a trifluoromethylsulfonyloxy group or a methylsulfonyloxy group).

As used herein, the term “protecting group” in relation to PG above refers to a grouping of atoms that masks, reduces or prevents reactivity of the nitrogen atom to which PG is attached. Examples of protecting groups may be found in “Greene's Protective Groups in Organic Synthesis”, (Wiley, 4^{th} ed. 2007). In one embodiment, PG may be a Boc or Cbz group.

In one embodiment, one of R²⁰ and R²² is OH and the other is Cl, Br, I, or an activated oxygen group (such as a sulfonyloxy group, including a toluenesulfonyloxy group, a trifluoromethylsulfonyloxy group or a methylsulfonyloxy group).

In one embodiment of step (b) the catalyst is a palladium catalyst. Exemplary palladium catalysts may comprise a palladium catalyst having a phosphine ligand. Exemplary catalysts may comprise tetrakis(triphenylphosphine)palladium, [1,1′ bis(diphenylphosphino)ferrocene]palladium(II) or (2-dicyclohexylphosphino-2′,4′,6′-triisopropyl-1,1′-biphenyl)[2-(2′-amino-1,1′-biphenyl)]palladium(II).

In one embodiment, step (b) provides a Suzuki coupling, a Negishi coupling, a Kumada coupling, a Stille coupling, a Heck coupling, or variants thereof; especially a Suzuki coupling, or a Heck coupling. Step (b) may provide a sp2-sp2 coupling reaction.

In one embodiment, R²³ is a metal or metalloid connected to a carbon atom in R⁴. The metal or metalloid may be selected from a boron, zinc or tin group or a Grignard reagent, especially a boron group (such as a boronic acid or ester). In one embodiment, R²¹ is a metal or metalloid connected to a carbon atom. The metal or metalloid may be selected from a boron, zinc or tin group or a Grignard reagent, especially a boron group (such as a boronic acid or ester).

In step (c) a skilled person would be able to identify appropriate conditions for removal of a protecting group. For example, if PG is Boc, then step (c) may comprise treatment with trifluoroacetic acid. If PG is Cbz, then step (c) may comprise treatment with hydrobromic acid.

In one embodiment, step (b) may comprise coupling R²⁴—R⁵ and

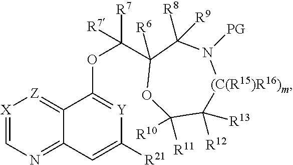

wherein R²⁴ is a leaving group (such as a halo (including fluoro, chloro, bromo or iodo) or an activated oxygen group (such as a sulfonyloxy group, including a toluenesulfonyloxy group, a trifluoromethylsulfonyloxy group or a methylsulfonyloxy group)), and wherein R⁵ is coupled to R⁴ via a nitrogen atom in R⁴. In this embodiment, R⁴ in step (c) is R⁴ which is substituted by R⁵.

Features of the second to ninth aspects of the present invention may be as described for the first aspect of the present invention. The medicament of the fifth and sixth aspects of the present invention may be a pharmaceutical composition, as described above.

Any of the features described herein can be combined in any combination with any one or more of the other features described herein within the scope of the invention.

Preferred features, embodiments and variations of the invention may be discerned from the following Examples which provides sufficient information for those skilled in the art to perform the invention. The following Examples are not to be regarded as limiting the scope of the preceding Summary of the Invention in any way.

### EXAMPLES

### Compound Synthesis

### Abbreviations

Throughout the Examples section, various abbreviations are used. While most would be understood by a person skilled in the art, an explanation of some of the abbreviations follow.
Bn: benzyl
Boc: t-butyloxycarbonyl
Cbz: carboxybenzyl
DMSO: dimethyl sulfoxide
HPLC: high performance liquid chromatography
H₂O: water
Hz: hertz
LCMS: liquid chromatography mass spectrometry
MeCN: acetonitrile
PG: protecting group
Prep: preparative
Rac: racemic
Rel: relative
UPLC: ultra performance liquid chromatography

### General Methods:

### Purification Methods:

### Method 1:

Silica gel chromatography techniques include either automated techniques or manual chromatography on pre-packed cartridges, manually packed flash columns or ionic solid phase extraction cartridges.

### Method 2:

Prep-HPLC was performed using the following conditions: Shimadzu UFLC XR Column: Xterra Prep MS C18 OBD, 19×150 mm, 10 microns. Column temperature: ambient temperature. Mobile Phase A: H₂O+0.05% formic acid. Mobile Phase B: MeCN. Flow rate: 15 mL/min. Mobile phase gradient and run time varied depending on the compound.

### Method 3:

Prep-HPLC was performed using the following conditions: Shimadzu UFLC XR. Column: Xterra Prep MS C18 OBD, 19×150 mm, 10 microns. Column temperature: ambient temperature. Mobile Phase A: H₂O+10 mM ammonium bicarbonate. Mobile Phase B: MeCN. Flow rate: 15 mL/min. Mobile phase gradient and run time varied depending on the compound.

### ¹H NMR Methods:

1H NMR spectra were recorded on Bruker AVANCE III HD 600 MHz or Varian 400 MHz spectrometers at 298 K in deuterated solvents indicated and referenced to residual solvent signals (1H: δ 7.26 for chloroform-d; 1H: δ 2.50 for DMSO-d6; 1H: δ 3.31 for methanol-d4, 1H: δ 4.79 for deuterium oxide). Abbreviations for the NMR data are as follows: s singlet, d=doublet, t=triplet, q=quartet, p=pentet, hept=heptet, m=multiplet, app=apparent, br=broad.

### LC MS Methods:

### Method 1:

Shimadzu LCMS-2020 Nexera UHPLC, Column: Xterra MS-C18, 2.1×50 mm, 2.5 micron. Column temperature: 40° C. Mobile Phase A: 1-1200.05% formic acid, Mobile Phase B: MeCN. Mobile phase gradient details: T=0 minutes (95% A, 5% B); T=0.3 minutes (95% A, 5% B); gradient to T:=3 minutes (5% A, 95% B); end of run at T=4 minutes (5% A, 95% B). Flow rate: 0.5 mL/min, analysis time 5.5 minutes. Detection method was UV at 254 nm as well as positive/negative mode electrospray ionisation on a Shimadzu LCMS-2020.

### Method 2:

Shimadzu LCMS-2020 Nexera UHPLC, Column: Xterra MS-C18, 2.1×50 mm, 3.5 micron. Column temperature: 40° C. Mobile Phase A: H₂O+0.05% formic acid, Mobile Phase B: MeCN. Mobile phase gradient details: T=0 minutes (95% A, 5% B); T=0.3 minutes (95% A, 5% B); gradient to T=3 minutes (5% A, 95% B); end of run at T=4 minutes (5% A, 95% B). Flow rate: 0.5 mL/min, analysis time 5.5 minutes. Detection method was UV at 254 nm as well as positive/negative mode electrospray ionisation on a Shimadzu LCMS-2020.

### Method 3:

Shimadzu LCM S-2020 Nexera UHPLC. Column: X-Bridge BEH C18, 2.1×50 mm, 2.5 micron. Column temperature: 40° C. Mobile Phase A: 10 mM ammonium bicarbonate. Mobile Phase B: MeCN. Mobile phase gradient details: T=0 minutes (95% A, 5% B); T=0.3 minutes (95% A, 5% B); gradient to T=3 minutes (5% A, 95% B); end of run at T=4 minutes (5% A, 95% B). Flow rate: 0.5 mL/min, analysis time 5.5 min. Detection method was UV at 254 nm as well as positive/negative mode electrospray ionisation on a Shimadzu LCMS-2020.

### Method 4:

Water Acquity UPLC with binary solvent manager with PDA detector and Acquity QDA performance mass detector. Column temperature: 35° C., auto sampler temperature: 5° C. Mobile Phase A: 0.1% Formic acid in Milli Q water (pH=2.70), Mobile Phase B: 0.1% Formic acid in water:MeCN (10:90). Mobile phase gradient details: T=0 min (97% A, 3% B) flow: 0.8 mL/min; T=0.75 min (97% A, 3% B) flow: 0.8 mL/min; gradient to T=2.7 min (2% A, 98% B) flow: 0.8 mL/min; gradient to T:=3 min (0% A, 100% B) flow: 1 mL/min; T=3.5 min (0% A, 100% B) flow: 1 mL/min; gradient to T=3.51 min (97% A, 3% B) flow: 0.8 mL/min; end of run at T=4 min (97% A, 3% B), Flow rate: 0.8 mL/min, analysis time 4 min. Column 1: X-Bridge C18 50×2.1 mm, 2.5 micron, Column 2: YMC tri-art C18 50×2.0 mm, 1.9 micron; Column 3: X-Bridge C18 50×4.6 mm, 3.5 micron; Column 4: Sunfire C18 150×4.6 mm, 3.5 micron; Column 5: YMC C18 50×2.0 mm, 1.9 micron; Column 6: X-Bridge C18 250×4.6 mm, 5.0 micron; Column 7: X-Bridge BEH C18 50×2.1 mm, 2.5 micron; Column 8: X-Bridge C18 50×2.5 mm, 2.5 micron; Column 9: Xtimate C18 50×2.1, 1.8 micron.

### Method 5:

Agilent 1200 LCMS 6130, Column: Atlantis dC18, 4.6×50 mm, 5 micron. Column temperature: 25° C. Mobile Phase A: H₂O+0.1% formic acid, Mobile Phase B: MeCN. Mobile phase gradient details: T=0 minutes (95% A, 5% B); T=2.5 minutes (5% A, 95% B); gradient to T=4 minutes (5% A, 95% B); end of run at T=4.5 minutes (95% A, 5% B). Flow rate: 1.5 mL/min, analysis time 6.0 min. UV detection: maximum absorption.

### Method 6:

Agilent 1290 Infinity II LCMS 6130, Column: X-Bridge C8, 4.6×50 mm, 3.5 micron. Column temperature: 25° C. Mobile Phase A: 10 mM ammonium bicarbonate in water, Mobile Phase B: MeCN. Mobile phase gradient details: T=0 minutes (95% A, 5% B); T=8.0 minutes (0% A, 100% B); gradient to T=8.1 minutes (0% A, 100% B); end of run at T=8.5 minutes (95% A, 5% B). Flow rate: 1.0 mL/min, analysis time 10.0 minutes. UV detection: maximum absorption.

### Method 7:

Agilent 1200 series. Column: X-Bridge C18 50×4.6 mm, 3.5 micron. Column temperature: 25° C. Mobile Phase A: 0.1% Formic acid in water, Mobile Phase B: MeCN. Mobile phase gradient details: T=0 minutes (95% A, 5% B); T:=8.0 minutes (0% A, 100% B); gradient to T=8.1 minutes (0% A, 100% B); end of run at T=8.5 minutes (95% A, 5% B). Flow rate: 1.0 mL/min, analysis time 10 minutes. UV detection: maximum absorption.

### Method 8:

Waters Alliance 2690 and 996 PDA detector with Micromass ZQ. Column Temperature: 25° C. Mobile Phase A: 5 mM ammonium acetate+0.1% formic acid in water (pH=3.5). Mobile Phase B: methanol. Gradient details: T=0 minutes (90% A, 10% B); T=7.0 minutes (10% A, 90% B); gradient to T=9 minutes (0% A, 100% B gradient to T=14 minutes (0% A, 100% B); gradient to T=14.1 minutes (90% A, 10% B); T=17.0 minutes (90% A, 10% B). Flow rate: 1 mL/min, analysis time 17 minutes. Column 1: Welch C18 4.6×150 mm, 5 micron; Column 2: Sunfire C18, 150×4.6 mm, 3.5 micron; Column 3: X-Bridge C18, 250×4.6 mm, 5 micron.

### Method 9:

Aquity with PDA detector and SQ Detector. Column: X-Bridge C18, 50×2.1 mm, 2.5 micron. Column temperature: 35° C., auto sampler temperature: 25° C. Mobile Phase A: 5 mM ammonium bicarbonate in water (pH=7.35). Mobile Phase B: acetonitrile. Mobile phase gradient details: T=0 min (97% A, 3% B); T=0.20 minutes (97% A, 3% B); gradient to T=2.7 minutes (2% A, 98% B); gradient to T=3 minutes (0% A, 100% B); T=3.5 minutes (0% A, 100% B); gradient to T=3.51 minutes (97% A, 3% B); end of run at T=4 minutes (97% A, 3% B). Flow rate: 0.5 mL/min, analysis time 4 minutes.

### HPLC Method:

Waters HPLC-e2695 with Waters 2998-PDA detector. Column: X-Bridge C18, 150×4.6 mm, 3.5 micron. Column temperature: room temperature, auto sampler temperature: 15° C. Mobile Phase A: 0.1% ammonia solution (25%) in Milli-Q water (pH˜9), Mobile Phase B: 100% acetonitrile. Mobile phase gradient details: T=0 minutes (90% A, 10% B; gradient to T=7 minutes (10% A, 90% B), gradient to T=9 minutes (0% A, 100% B); T=14 minutes (0% A, 100% B); gradient to T=14.01 minutes (90% A, 10% B); end of run at T=17 minutes (90% A, 10% B), Flow rate: 1 mL/min, analysis time 17 minutes.

### General Schemes

### General Scheme Overview

Compounds of Formula (I) may be prepared by the general process described below. The methods of preparation may involve substitution or alkylation of a bicyclic compound to provide an intermediate which is then coupled in a sp2-sp2 coupling reaction to install the R⁴ group. The R⁴ group may be modified (for example to provide an R⁵ group) before removing the protecting group (PG) on the nitrogen.

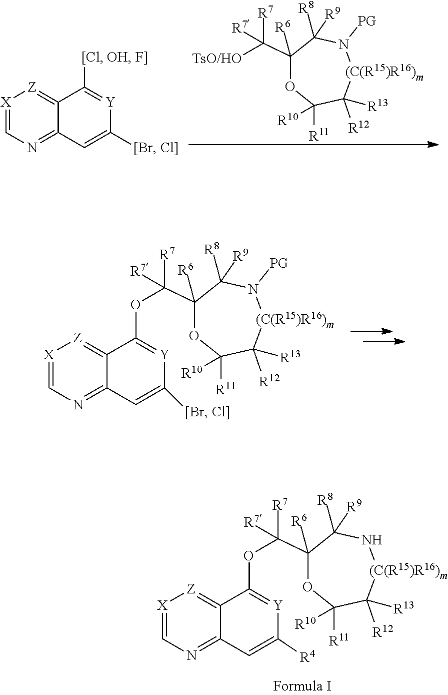

Various compounds of Formula (I) may be prepared using the synthetic schemes described below, employing techniques available in the art using readily available starting materials. The methods described may be readily adapted to provide other compounds which fall within the scope of Formula (I).

The following examples are intended to illustrate embodiments and should not be construed to be limiting in any way. Additional compounds may be prepared using similar reaction schemes and methods.

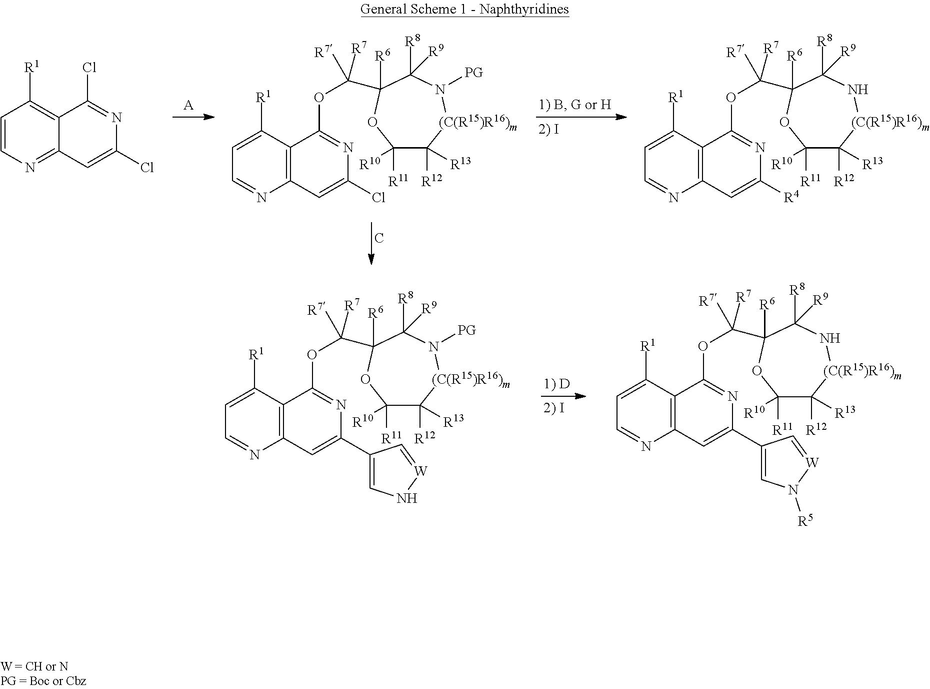

In the above scheme, the step marked “A”, for example, is General Procedure A below. Similarly, the step marked “B’ is General Procedure B below, and so on. Substituents are as defined above. PG is a protecting group.

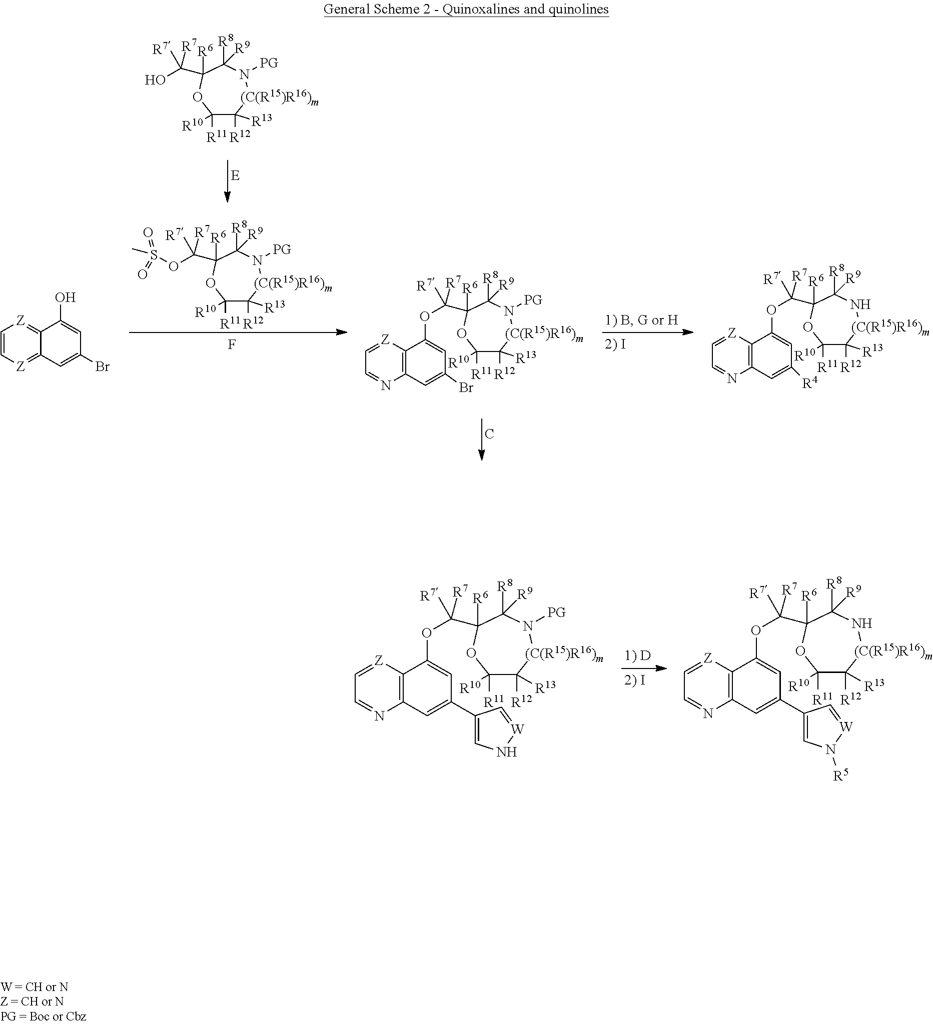

In the above scheme, the step marked “E”, for example, is General Procedure E below. Similarly, the step marked “F’ is General Procedure F below, and so on. Substituents are as defined above. PG is a protecting group.

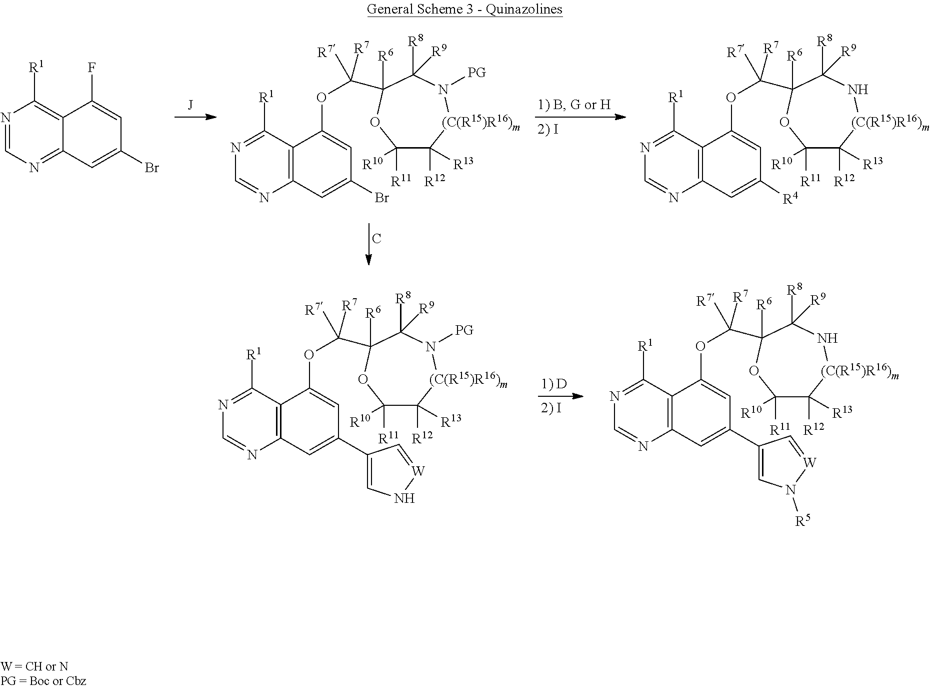

In the above scheme, the step marked “J”, for example, is General Procedure J below. Substituents are as defined above. PG is a protecting group.

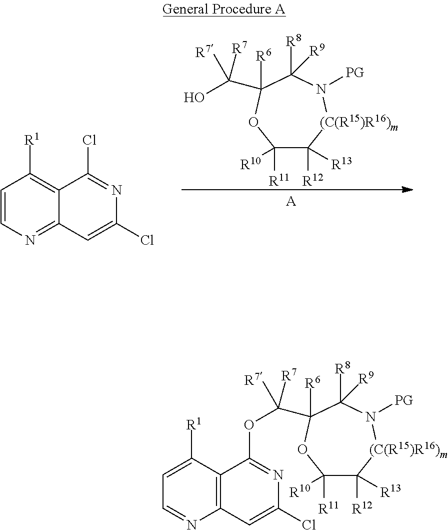

To a solution of alcohol (1.2 eq) in acetonitrile (0.05-0.1 M) cooled to 0° C. was added sodium hydride, 57-63% oil dispersion (2.0 eq) portion-wise and the reaction was stirred for 5 minutes. After this time, the appropriate heteroaryl chloride (1.0 eq) was added and the reaction was stirred at room temperature for 1-5 hours.

The reaction mixture was quenched by slow addition of water and the products were extracted with ethyl acetate. The combined organics were washed with brine, dried over anhydrous sodium/magnesium sulfate and concentrated. The crude material was purified using purification method 1.

TABLE 1
Analytical data for intermediates synthesised by general method A

| | | | | LC |
|---|---|---|---|---|
| | | | | MS |
| | | | | Meth- |
| No | Structure | 1H NMR | LC MS RT, m/z | od |
| 1.1 | 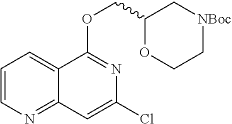 [SMILES: CC(C)(C)OC(=O)N1CCOC(COc2nc(Cl)cc3ncccc23)C1] | 1H NMR (400 MHz, Methanol-d4) δ 9.01 (dd, 1H), 8.66 (d, 1H), 7.61 (dd, 1H), 7.49 (d, 1H), 4.64-4.53 (m, 2H), 4.08 (d, 1H), 3.97-3.78 (m, 3H), 3.63-3.55 (m, 1H), 3.11-2.84 (m, 2H), 1.46 (s, 9H). | 2.98 min, [MH]+ = 380 | 1 |
| 1.2 | 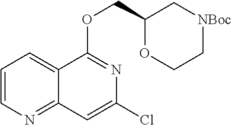 [SMILES: CC(C)(C)OC(=O)N1CCO[C@@H](COc2nc(Cl)cc3ncccc23)C1] | 1H NMR (400 MHz, Methanol-d4) δ 9.00 (dd, 1H), 8.65 (d, 1H), 7.61 (dd, 1H), 7.48 (d, 1H), 4.62-4.54 (m, 2H), 4.08 (d, 1H), 3.96-3.82 (m, 3H), 3.58 (td, 1H), 3.14-2.84 (m, 2H), 1.46 (s, 9H). | 2.97 min, [MH]+ = 380 | 1 |
| 1.3 | 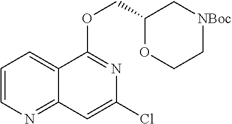 [SMILES: CC(C)(C)OC(=O)N1CCO[C@H](COc2nc(Cl)cc3ncccc23)C1] | 1H NMR (400 MHz, Methanol-d4) δ 8.99 (dd, 1H), 8.63 (d, 1H), 7.60 (dd, 1H), 7.47 (d, 1H), 4.64-4.51 (m, 2H), 4.08 (d, 1H), 3.98-3.79 (m, 3H), 3.58 (td, 1H), 3.03 (s, 2H), 1.46 (s, 9H). | 2.96 min, [MH]+ = 380 | 1 |
| 1.4 | 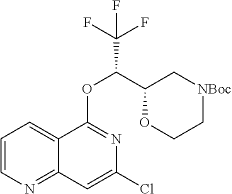 [SMILES: CC(C)(C)OC(=O)N1CCO[C@H]([C@H](Oc2nc(Cl)cc3ncccc23)C(F)(F)F)C1] | No NMR recorded. | 3.21 min, [MH]+ = 448 | 2 |
| 1.5 | 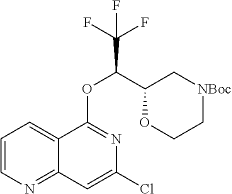 [SMILES: CC(C)(C)OC(=O)N1CCO[C@H]([C@@H](Oc2nc(Cl)cc3ncccc23)C(F)(F)F)C1] | No NMR recorded | 3.25 min, [MH]+ = 448 | 2 |
| 1.6 | 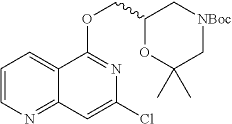 [SMILES: CC(C)(C)OC(=O)N1CC(COc2nc(Cl)cc3ncccc23)OC(C)(C)C1] | 1H NMR (400 MHz, Methanol-d4) δ 9.01 (dd, 1H), 8.65 (s, 1H), 7.62 (dd, 1H), 7.49 (d, 1H), 4.62-4.41 (m, 2H), 4.29-4.06 (m, 2H), 3.79 (d, 1H), 3.01-2.56 (m, 2H), 1.47 (s, 9H), 1.26 (s, 3H), 1.22 (s, 3H). | 3.30 min, [MH]+ = 408 | 2 |
| 1.7 | 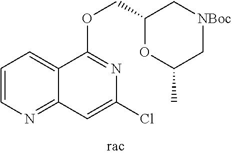 [SMILES: C[C@H]1CN(C(=O)OC(C)(C)C)C[C@@H](COc2nc(Cl)cc3ncccc23)O1] | 1H NMR (400 MHz, Methanol-d4) δ 9.00 (ddd, 1H), 8.64 (d, 1H), 7.60 (ddd, 1H), 7.49-7.45 (m, 1H), 4.63-4.49 (m, 2H), 4.12 (d, 1H), 4.00-3.89 (m, 2H), 3.68-3.56 (m, 1H), 2.83 (br s, 1H), 2.57 (br s, 1H), 1.46 (s, 9H), 1.19 (d, 3H). | 3.19 min, [MH]+ = 394 | 2 |
| 1.8 | 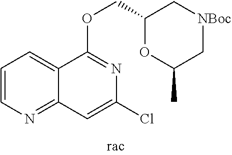 [SMILES: C[C@@H]1CN(C(=O)OC(C)(C)C)C[C@@H](COc2nc(Cl)cc3ncccc23)O1] | 1H NMR (400 MHz, Methanol-d4) δ 9.01 (dd, 1H), 8.66 (d, 1H), 7.61 (dd, 1H), 7.49 (s, 1H), 4.82-4.52 (m, 2H), 4.33-4.24 (m, 1H), 4.11-3.99 (m, 1H), 3.88-3.43 (m, 3H), 3.16-2.80 (m, 1H), 1.58-1.23 (m, 9H), 1.15 (d, 3H). | 3.08 min, [MH]+ = 394 | 2 |
| 1.9 | 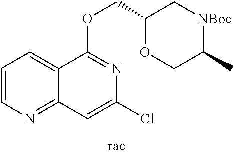 [SMILES: C[C@H]1CO[C@H](COc2nc(Cl)cc3ncccc23)CN1C(=O)OC(C)(C)C] | 1H NMR (400 MHz, Methanol-d4) δ 9.06-8.92 (m, 1H), 8.69-8.55 (m, 1H), 7.64-7.57 (m, 1H), 7.51-7.43 (m, 1H), 4.59 (s, 2H), 4.13-4.05 (m, 1H), 4.01-3.83 (m, 2H), 3.82-3.67 (m, 2H), 3.09 (br s, 1H), 1.47 (s, 9H), 1.27 (d, 3H). | 3.15 min, [MH]+ = 394 | 2 |
| 1.10 | 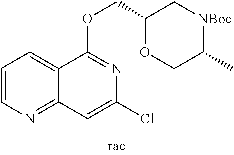 [SMILES: C[C@@H]1CO[C@H](COc2nc(Cl)cc3ncccc23)CN1C(=O)OC(C)(C)C] | 1H NMR (400 MHz, Methanol-d4) δ 9.06-8.95 (m, 1H), 8.68-8.60 (m, 1H), 7.60 (ddd, 1H), 7.50-7.45 (m, 1H), 4.76 (ddd, 1H), 4.60 (ddd, 1H), 4.31 (q, 1H), 4.16-4.06 (m, 1H), 4.01 (dd, 1H), 3.89 (dd, 1H), 3.55-3.43 (m, 2H), 1.37 (s, 9H), 1.26 (d, 3H). | 3.04 min, [MH]+ = 394 | 2 |
| 1.11 | 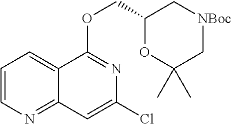 [SMILES: CC(C)(C)OC(=O)N1C[C@@H](COc2nc(Cl)cc3ncccc23)OC(C)(C)C1] | 1H NMR (400 MHz, Methanol-d4) δ 9.00 (dd, 1H), 8.64 (d, 1H), 7.61 (dd, 1H), 7.48 (d, 1H), 4.58-4.42 (m, 2H), 4.27-4.08 (m, 2H), 3.79 (dd, 1H), 2.80 (br s, 2H), 1.46 (s, 9H), 1.26 (s, 3H), 1.22 (s, 3H). | 3.30 min, [MH]+ = 408 | 2 |
| 1.12 | 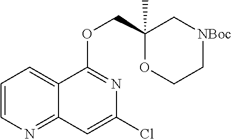 [SMILES: CC(C)(C)OC(=O)N1CCO[C@@](C)(COc2nc(Cl)cc3ncccc23)C1] | No NMR recorded. | 3.02 min, [MH]+ = 394 | 2 |
| 1.13 | 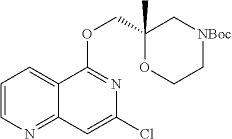 [SMILES: CC(C)(C)OC(=O)N1CCO[C@](C)(COc2nc(Cl)cc3ncccc23)C1] | No NMR recorded. | 3.04 min, [MH]+ = 394 | 2 |
| 1.14 | 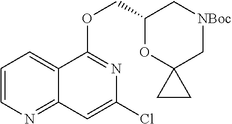 [SMILES: CC(C)(C)OC(=O)N1C[C@@H](COc2nc(Cl)cc3ncccc23)OC2(CC2)C1] | 1H NMR (400 MHz, Chloroform-d) δ 9.01 (dd, 1H), 8.49 (ddd, 1H), 7.54 (d, 1H), 7.44 (dd, 1H), 4.72-4.45 (m, 2H), 4.37-3.94 (m, 2H), 3.65-3.23 (m, 2H), 3.03 (s, 1H), 1.47 (s, 9H), 1.06-0.94 (m, 1H), 0.80-0.53 (m, 3H). | 3.28 min, [MH]+ = 406 | 2 |
| 1.15 | 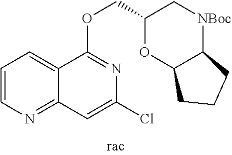 [SMILES: CC(C)(C)OC(=O)N1C[C@@H](COc2nc(Cl)cc3ncccc23)O[C@@H]2CCC[C@@H]21] | 1H NMR (400 MHz, Methanol-d4) δ 9.00 (dd, 1H), 8.69-8.60 (m, 1H), 7.61 (ddd, 1H), 7.48 (s, 1H), 4.92-4.64 (m, 2H), 4.28 (t, 2H), 4.19 (s, 1H), 3.87 (d, 1H), 3.40 (d, 1H), 1.96-1.78 (m, 4H), 1.78-1.57 (m, 2H), 1.36 (br s, 9H). | 3.23 min, [MH]+ = 420 | 2 |
| 1.16 | 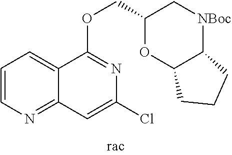 [SMILES: CC(C)(C)OC(=O)N1C[C@@H](COc2nc(Cl)cc3ncccc23)O[C@H]2CCC[C@H]21] | 1H NMR (400 MHz, Methanol-d4) δ 9.04-8.96 (m, 1H), 8.70-8.55 (m, 1H), 7.65-7.57 (m, 1H), 7.51-7.44 (m, 1H), 4.65-4.50 (m, 2H), 4.18- 4.02 (m, 1H), 3.99-3.85 (m, 3H), 2.99 (dt, 1H), 1.97-1.70 (m, 5H), 1.69- 1.56 (m, 1H), 1.47 (s, 9H). | 3.35 min, [MH]+ = 420 | 2 |
| 1.17 | 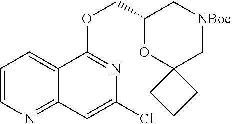 [SMILES: CC(C)(C)OC(=O)N1C[C@@H](COc2nc(Cl)cc3ncccc23)OC2(CCC2)C1] | No NMR recorded. | 3.34 min, [MH]+ = 420 | 2 |
| 1.18 | 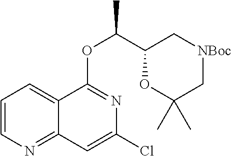 [SMILES: C[C@H](Oc1nc(Cl)cc2ncccc12)[C@@H]1CN(C(=O)OC(C)(C)C)CC(C)(C)O1] | 1H NMR (400 MHz, Methanol-d4) δ 8.99 (dd, 1H), 8.62 (d, 1H), 7.60 (dd, 1H), 7.45 (d, 1H), 5.48 (qd, 1H), 4.13-3.93 (m, 2H), 3.76 (d, 1H), 2.93-2.53 (m, 2H), 1.53-1.34 (m, 12H), 1.19 (d, 6H). | 3.43 min, [MH]+ = 422 | 2 |
| 1.19 | 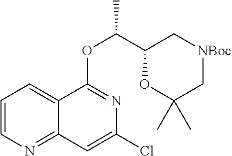 [SMILES: C[C@@H](Oc1nc(Cl)cc2ncccc12)[C@@H]1CN(C(=O)OC(C)(C)C)CC(C)(C)O1] | 1H NMR (400 MHz, Methanol-d4) δ 9.00 (dd, 1H), 8.64 (s, 1H), 7.64- 7.57 (m, 1H), 7.46 (d, 1H), 5.35 (p, 1H), 4.32-4.07 (m, 1H), 3.92 (ddd, 1H), 3.76 (dd, 1H), 2.94-2.56 (m, 2H), 1.48-1.42 (m, 12H), 1.23 (s, 3H), 1.18 (s, 3H). | 3.43 min, [MH]+ = 422 | 2 |
| 1.20 | 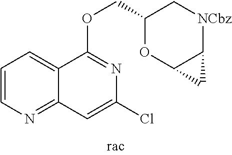 [SMILES: O=C(OCc1ccccc1)N1C[C@@H](COc2nc(Cl)cc3ncccc23)O[C@H]2C[C@H]21] | 1H NMR (400 MHz, Chloroform-d) δ 9.01 (dd, 1H), 8.56-8.43 (m, 1H), 7.55 (s, 1H), 7.49-7.29 (m, 6H), 5.30-5.12 (m, 2H), 4.72 (dt, 1H), 4.61- 4.51 (m, 1H), 4.15-4.06 (m, 1H), 3.97-3.86 (m, 1H), 3.85-3.74 (m, 1H), 3.25-3.01 (m, 1H), 2.99-2.83 (m, 1H), 1.01-0.84 (m, 2H). | 3.04 min, [MH]+ = 426 | 2 |
| 1.21 | 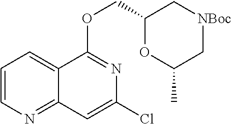 [SMILES: C[C@H]1CN(C(=O)OC(C)(C)C)C[C@@H](COc2nc(Cl)cc3ncccc23)O1] | 1H NMR (400 MHz, Methanol-d4) δ 9.00 (dd, 1H), 8.65 (d, 1H), 7.61 (dd, 1H), 7.48 (d, 1H), 4.65-4.50 (m, 2H), 4.12 (d, 1H), 4.00-3.86 (m, 2H), 3.68-3.57 (m, 1H), 2.83 (br s, 1H), 2.58 (br s, 1H), 1.46 (s, 9H), 1.19 (d, 3H). | 3.19 min, [MH]+ = 394 | 2 |
| 1.22 | 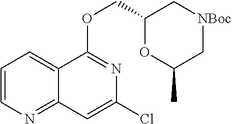 [SMILES: C[C@@H]1CN(C(=O)OC(C)(C)C)C[C@@H](COc2nc(Cl)cc3ncccc23)O1] | 1H NMR (400 MHz, Methanol-d4) δ 9.01 (dd, 1H), 8.66 (d, 1H), 7.61 (dd, 1H), 7.49 (s, 1H), 4.80-4.46 (m, 2H), 4.34-4.25 (m, 1H), 4.13- 3.98 (m, 1H), 3.91-3.42 (m, 3H), 3.20-2.71 (m, 1H), 1.68-1.24 (m, 9H), 1.16 (d, 3H). | 3.08 min, [MH]+ = 394 | 2 |
| 1.23 | 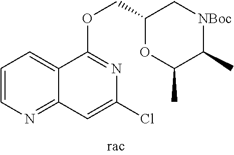 [SMILES: C[C@H]1O[C@H](COc2nc(Cl)cc3ncccc23)CN(C(=O)OC(C)(C)C)[C@H]1C] | 1H NMR (400 MHz, Methanol-d4) δ 8.99 (dd, 1H), 8.67-8.59 (m, 1H), 7.60 (dd, 1H), 7.47 (s, 1H), 4.95-4.84 (m, 1H), 4.70-4.59 (m, 1H), 4.35- 4.25 (m, 1H), 4.19 (qd, 1H), 4.05-3.96 (m, 1H), 3.88 (ddd, 1H), 3.40 (dd, 1H), 1.38 (s, 9H), 1.16 (d, 3H), 1.03 (d, 3H). | 3.20 min, [MH]+ = 408 | 2 |
| 1.24 | 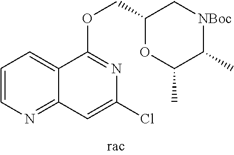 [SMILES: C[C@@H]1O[C@H](COc2nc(Cl)cc3ncccc23)CN(C(=O)OC(C)(C)C)[C@@H]1C] | 1H NMR (400 MHz, Methanol-d4) mixture of rotamers: δ 9.01 (dd, 1H), 8.68-8.58 (m, 1H), 7.62 (dd, 1H), 7.49 (s, 1H), 4.66-4.48 (m, 2H), 4.03-3.87 (m, 3H), 3.83-3.72 (m, 1H), 3.12-2.91 (m, 1H), 1.52-1.43 (m, 9H), 1.20-1.08 (m, 6H). | 3.30 min, [MH]+ = 408 | 2 |
| 1.25 | 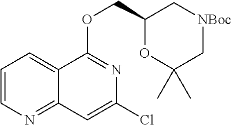 [SMILES: CC(C)(C)OC(=O)N1C[C@H](COc2nc(Cl)cc3ncccc23)OC(C)(C)C1] | 1H NMR (400 MHz, Methanol-d4) δ 9.00 (dd, 1H), 8.64 (d, 1H), 7.61 (dd, 1H), 7.48 (d, 1H), 4.59-4.43 (m, 2H), 4.26-4.07 (m, 2H), 3.79 (d, 1H), 2.93-2.64 (m, 2H), 1.47 (s, 9H), 1.26 (s, 3H), 1.22 (s, 3H). | 3.29 min, [MH]+ = 408 | 2 |
| 1.26 | 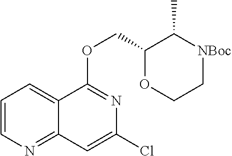 [SMILES: C[C@H]1[C@@H](COc2nc(Cl)cc3ncccc23)OCCN1C(=O)OC(C)(C)C] | 1H NMR (400 MHz, Chloroform-d) δ 9.01 (dd, 1H), 8.52 (ddd, 1H), 7.54 (s, 1H), 7.44 (dd, 1H), 4.65-4.35 (m, 2H), 4.23-3.88 (m, 2H), 3.87-3.67 (m, 1H), 3.60 (t, 1H), 3.49 (d, 1H), 3.34-3.06 (m, 1H), 1.48 (s, 9H), 1.22 (d, 3H). | 3.14 min, [MH]+ = 394 | 2 |
| 1.27 | 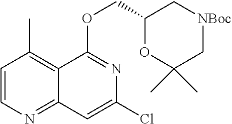 [SMILES: Cc1ccnc2cc(Cl)nc(OC[C@@H]3CN(C(=O)OC(C)(C)C)CC(C)(C)O3)c12] | 1H NMR (400 MHz, Methanol-d4) δ 8.74 (d, 1H), 7.41 (s, 1H), 7.40- 7.37 (m, 1H), 4.52-4.38 (m, 2H), 4.25-4.11 (m, 2H), 3.81 (d, 1H), 2.92 (d, 3H), 2.89-2.59 (m, 2H), 1.47 (s, 9H), 1.26 (s, 3H), 1.23 (s, 3H). | 3.14 min, [MH]+ = 394 | 2 |
| 1.28 | 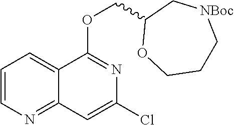 [SMILES: CC(C)(C)OC(=O)N1CCCOC(COc2nc(Cl)cc3ncccc23)C1] | 1H NMR (600 MHz, Chloroform-d) mixture of rotamers: δ 9.06-8.96 (m, 1H), 8.54 (dd, 1H), 7.54 (d, 1H), 7.48-7.41 (m, 1H), 4.64- 4.50 (m, 2H), 4.20-3.88 (m, 3H), 3.81-3.72 (m, 1H), 3.63-3.53 (m, 1H), 3.43-3.35 (m, 0.5H), 3.34-3.22 (m, 1H), 3.14 (dd, 0.5H), 2.03-1.89 (m, 2H), 1.52-1.44 (m, 9H). | 3.03 min, [MH]+ = 394 | 2 |
| 1.29 | 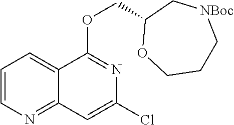 [SMILES: CC(C)(C)OC(=O)N1CCCO[C@H](COc2nc(Cl)cc3ncccc23)C1] | 1H NMR (400 MHz, Methanol-d4) mixture of rotamers: δ 9.00 (d, 1H), 8.67 (dd, 1H), 7.61 (dd, 1H), 7.48 (d, 1H), 4.67-4.57 (m, 1H), 4.57- 4.43 (m, 1H), 4.19-4.04 (m, 2H), 4.01-3.83 (m, 1H), 3.74-3.52 (m, 2H), 3.52-3.32 (m, 2H), 2.00-1.79 (m, 2H), 1.51-1.39 (m, 9H). | 3.02 min, [MH]+ = 394 | 2 |
| 1.30 | 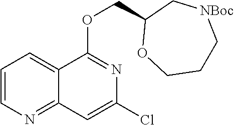 [SMILES: CC(C)(C)OC(=O)N1CCCO[C@@H](COc2nc(Cl)cc3ncccc23)C1] | 1H NMR (400 MHz, Methanol-d4) mixture of rotamers δ 9.00 (d, 1H), 8.66 (dd, 1H), 7.61 (dd, 1H), 7.48 (d, 1H), 4.68-4.55 (m, 1H), 4.55- 4.45 (m, 1H), 4.20-4.04 (m, 2H), 3.96 (dd, 0.5H), 3.88 (dd, 0.5H), 3.74- 3.53 (m, 2H), 3.53-3.32 (m, 2H), 1.97-1.82 (m, 2H), 1.48 (s, 4.5H), 1.43 (s, 4.5H). | 3.08 min, [MH]+ = 394 | 2 |
| 1.31 | 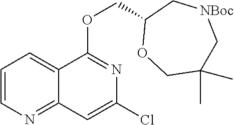 [SMILES: CC1(C)CO[C@H](COc2nc(Cl)cc3ncccc23)CN(C(=O)OC(C)(C)C)C1] | 1H NMR (600 MHz, Methanol-d4) mixture of rotamers δ 9.04-8.96 (m, 1H), 8.66 (dd, 1H), 7.61 (ddd, 1H), 7.48-7.44 (m, 1H), 4.64-4.58 (m, 1H), 4.52-4.44 (m, 1H), 4.27-4.17 (m, 1H), 3.92-3.84 (m, 0.5H), 3.79- 3.70 (m, 0.5H), 3.69-3.57 (m, 1.5H), 3.50-3.33 (m, 2.5H), 3.25- 3.16 (m, 1H), 1.48 (s, 4.5H), 1.46 (s, 4.5H), 0.98 (s, 3H), 0.92 (s, 1.5H), 0.88 (s, 1.5H). | 3.33 min, [MH]+ = 422 | 2 |
| 1.32 | 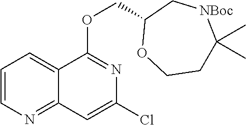 [SMILES: CC(C)(C)OC(=O)N1C[C@@H](COc2nc(Cl)cc3ncccc23)OCCC1(C)C] | 1H NMR (400 MHz, Chloroform-d) δ 9.00 (dd, 1H), 8.57 (ddd, 1H), 7.52 (d, 1H), 7.43 (dd, 1H), 4.63 (dd, 1H), 4.55 (dd, 1H), 4.05 (ddd, 1H), 4.00-391 (m, 1H), 3.82 (ddd, 1H), 3.39 (dd, 1H), 2.15 (dd, 1H), 1.88-1.78 (m, 1H), 1.55 (s, 3H), 1.46 (s, 9H), 1.41 (s, 3H). | 3.36 min, [MH]+ = 422 | 2 |
| 1.33 | 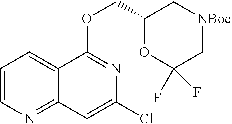 [SMILES: CC(C)(C)OC(=O)N1C[C@@H](COc2nc(Cl)cc3ncccc23)OC(F)(F)C1] | 1H NMR (400 MHz, Chloroform-d) δ 9.02 (dd, 1H), 8.50 (ddd, 1H), 7.56 (d, 1H), 7.45 (dd, 1H), 4.77-4.62 (m, 2H), 4.62-4.49 (m, 1H), 4.41-3.90 (m, 2H), 3.33-2.87 (m, 2H), 1.48 (s, 9H). | 3.18 min, [MH]+ = 416 | 2 |
| 1.34 | 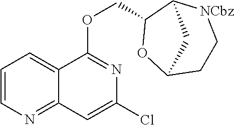 [SMILES: O=C(OCc1ccccc1)N1CC[C@@H]2C[C@H]1[C@@H](COc1nc(Cl)cc3ncccc13)O2] | 1H NMR (400 MHz, Chloroform-d) mixture of rotamers δ 9.02-8.94 (m, 1H), 8.58-8.51 (m, 0.5H), 8.42 (ddd, 0.5H), 7.57-7.51 (m, 1H), 7.46- 7.37 (m, 1H), 7.33-7.20 (m, 5H), 5.14-5.03 (m, 2.5H), 4.95-4.88 (m, 0.5H), 4.81 (dd, 0.5H), 4.75-4.56 (m, 2.5H), 4.45 (ddd, 0.5H), 4.39 (ddd, 0.5H), 4.02-3.87 (m, 1H), 3.54-3.41 (m, 1H), 2.22-2.10 (m, 1H), 2.00-1.89 (m, 1H), 1.89-1.83 (m, 1H), 1.79-1.66 (m, 1H). | 2.96 min, [MH]+ = 440 | 2 |
| | or | | | |
| | 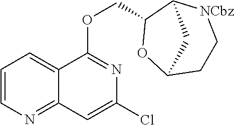 [SMILES: O=C(OCc1ccccc1)N1CC[C@@H]2C[C@H]1[C@@H](COc1nc(Cl)cc3ncccc13)O2] | | | |
| | (Absolute stereochemistry | | | |
| | not determined) | | | |
| 1.35 | 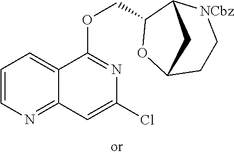 [SMILES: O=C(OCc1ccccc1)N1CC[C@H]2C[C@@H]1[C@@H](COc1nc(Cl)cc3ncccc13)O2] | 1H NMR (400 MHz, Chloroform-d) mixture of rotamers δ 9.00 (d, 1H), 8.55 (d, 0.5H), 8.40 (d, 0.5H), 7.54 (d, 0.5H), 7.50-7.41 (m, 0.5H), 7.41- 7.24 (m, 6H), 5.21-5.08 (m, 2H), 5.04-4.91 (m, 0.5H), 4.91-4.78 (m, 0.5H), 4.69-4.62 (m, 1H), 4.60-4.45 (m, 2H), 4.45-4.33 (m, 1H), 4.14-3.97 (m, 1H), 3.44-3.27 (m, 1H), 2.18-2.02 (m, 1H), 1.93-1.75 (m, 2H), 1.75-1.63 (m, 1H). | 3.02 min, [MH]+ = 440 | 2 |
| | 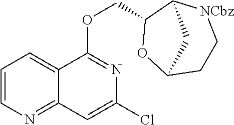 [SMILES: O=C(OCc1ccccc1)N1CC[C@@H]2C[C@H]1[C@@H](COc1nc(Cl)cc3ncccc13)O2] | | | |
| | (Absolute stereochemistry | | | |
| | not determined) | | | |
| 1.36 | 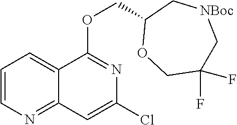 [SMILES: CC(C)(C)OC(=O)N1C[C@@H](COc2nc(Cl)cc3ncccc23)OCC(F)(F)C1] | 1H NMR (400 MHz, Methanol-d4) δ 9.00 (dd, 1H), 8.70-8.58 (m, 1H), 7.61 (dd, 1H), 7.48 (s, 1H), 4.62 (dd, 1H), 4.53 (dd, 1H), 4.30-4.04 (m, 4H), 3.92-3.74 (m, 1H), 3.74-3.54 (m, 1H), 3.49-3.18 (m, 1H), 1.53- 1.41 (m, 9H). | 3.16 min, [MH]+ = 430 | 2 |
| 1.37 | 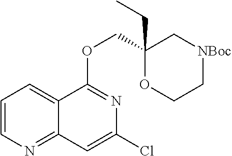 [SMILES: CC[C@@]1(COc2nc(Cl)cc3ncccc23)CN(C(=O)OC(C)(C)C)CCO1] | 1H NMR (400 MHz, Chloroform-d) δ 9.01 (dd, 1H), 8.50 (d, 1H), 7.54 (s, 1H), 7.44 (dd, 1H), 4.75-4.30 (m, 2H), 3.91-3.71 (m, 2H), 3.63-3.35 (m, 4H), 2.03-1.83 (m, 1H), 1.83-1.64 (m, 1H), 1.51-1.33 (m, 9H), 0.98 (t, 3H). | 3.22 min, [MH]+ = 408 | 2 |
| 1.38 | 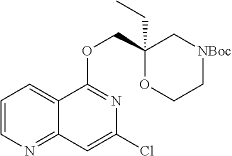 [SMILES: CC[C@]1(COc2nc(Cl)cc3ncccc23)CN(C(=O)OC(C)(C)C)CCO1] | 1H NMR (400 MHz, Chloroform-d) δ 9.01 (dd, 1H), 8.50 (d, 1H), 7.54 (s, 1H), 7.44 (dd, 1H), 4.72-4.39 (m, 2H), 3.86-3.72 (m, 2H), 3.62- 3.38 (m, 4H), 1.98-1.81 (m, 1H), 1.81-1.65 (m, 1H), 1.52-1.30 (m, 9H), 0.98 (t, 3H). | 3.22 min, [MH]+ = 408 | 2 |
| 1.39 | 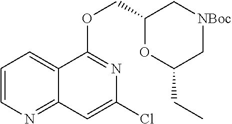 [SMILES: CC[C@H]1CN(C(=O)OC(C)(C)C)C[C@@H](COc2nc(Cl)cc3ncccc23)O1] | 1H NMR (400 MHz, Methanol-d4) δ 9.01 (ddd, 1H), 8.64 (d, J = 8.3 Hz, 1H), 7.65-7.57 (m, 1H), 7.52-7.44 (m, 1H), 4.66-4.52 (m, 2H), 4.13 (d, 1H), 4.01-3.88 (m, 2H), 3.46-3.36 (m, 1H), 2.98-2.73 (m, 1H), 2.73-2.47 (m, 1H), 1.60-1.49 (m, 2H), 0.97 (t, 3H). | 3.37 min, [MH]+ = 408 | 2 |
| 1.40 | 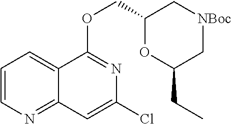 [SMILES: CC[C@@H]1CN(C(=O)OC(C)(C)C)C[C@@H](COc2nc(Cl)cc3ncccc23)O1] | 1H NMR (400 MHz, Methanol-d4) δ 9.01 (dd, 1H), 8.65 (ddd, 1H), 7.62 (dd, 1H), 7.49 (d, 1H), 4.82-4.70 (m, 1H), 4.70-4.49 (m, 1H), 4.32- 4.23 (m, 1H), 3.82-3.45 (m, 4H), 3.26-2.89 (m, 1H), 1.65-1.22 (m, 13H), 0.90 (t, 3H). | 3.24 min, [MH]+ = 408 | 2 |
| 1.41 | 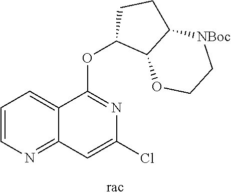 | 1H NMR (400 MHz, Methanol-d4) δ 9.00 (dd, 1H), 8.68-8.62 (m, 1H), 7.61 (ddd, 1H), 7.50-7.43 (m, 1H), 5.47-5.35 (m, 1H), 4.03 (ddd, 1H), 3.89 (ddd, 1H), 3.80 (dd, 1H), 3.72 (td, 1H), 3.16-3.05 (m, 1H), 2.97 (ddd, 1H), 2.63-2.48 (m, 2H), 2.17-2.03 (m, 1H), 1.78-1.61 (m, 1H), 1.48 (s, 9H). | 3.22 min, [MH]+ = 406 | 2 |
| 1.42 | 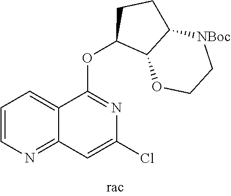 | 1H NMR (400 MHz, Chloroform-d) δ 9.00 (dd, 1H), 8.44 (ddd, 1H), 7.52 (d, 1H), 7.43 (dd, 1H), 5.58-5.44 (m, 1H), 4.68-4.24 (m, 1H), 3.96 (d, 1H), 3.94-3.78 (m, 1H), 3.78-3.62 (m, 1H), 3.51 (td, 1H), 3.24-3.01 (m, 1H), 2.66-2.52 (m, 1H), 2.14-1.96 (m, 1H), 1.96-1.74 (m, 2H), 1.49 (s, 9H). | 3.24 min, [MH]+ = 406 | 2 |
| 1.43 | 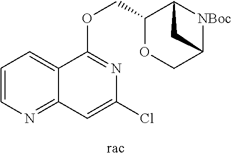 [SMILES: CC(C)(C)OC(=O)N1[C@@H]2CO[C@H](COc3nc(Cl)cc4ncccc34)[C@H]1C2] | 1H NMR (600 MHz, Methanol-d4) δ 9.00 (dd, 1H), 8.67 (ddd, 1H), 7.61 (dd, 1H), 7.48 (s, 1H), 4.85-4.79 (m, 1H), 4.68-4.56 (m, 1H), 4.43-4.33 (m, 2H), 4.30 (d, 1H), 4.24-4.15 (m, 1H), 3.87 (d, 1H), 2.86-2.78 (m, 1H), 1.81-1.71 (m, 1H), 1.47 (s, 9H). | 2.91 min, [MH]+ = 392 | 2 |
| 1.44 | 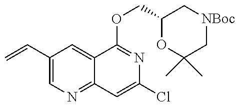 [SMILES: C=Cc1cnc2cc(Cl)nc(OC[C@@H]3CN(C(=O)OC(C)(C)C)CC(C)(C)O3)c2c1] | 1H NMR (400 MHz, Chloroform-d) δ 9.09 (d, 1H), 8.38 (d, 1H), 7.50 (d, 1H), 6.85 (dd, 1H), 5.99 (d, 1H), 5.51 (d, 1H), 4.56 (dd, 1H), 4.48 (dd, 1H), 4.34-4.03 (m, 2H), 3.95-3.67 (m, 1H), 2.85-2.56 (m, 2H), 1.48 (s, 9H), 1.28 (s, 3H), 1.25 (s, 3H). | 3.49 min, [MH]+ = 434 | 2 |
| 1.45 | 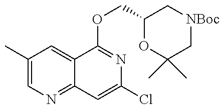 [SMILES: Cc1cnc2cc(Cl)nc(OC[C@@H]3CN(C(=O)OC(C)(C)C)CC(C)(C)O3)c2c1] | 1H NMR (600 MHz, Chloroform-d) δ 8.85 (d, 1H), 8.27-8.21 (m, 1H), 7.49 (s, 1H), 4.62-4.52 (m, 1H), 4.52-4.40 (m, 1H), 4.37-4.02 (m, 2H), 3.94-3.67 (m, 1H), 2.86-2.62 (m, 2H), 2.53 (s, 3H), 1.48 (s, 9H), 1.28 (s, 3H), 1.25 (s, 3H). | No LC MS recorded | — |
| 1.46 | 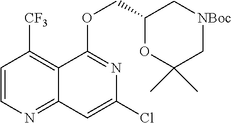 [SMILES: CC(C)(C)OC(=O)N1C[C@@H](COc2nc(Cl)cc3nccc(C(F)(F)F)c23)OC(C)(C)C1] | 1H NMR (400 MHz, Methanol-d4) δ 9.19 (d, 1H), 7.98 (dd, 1H), 7.68 (s, 1H), 4.65 (dd, 1H), 4.55-4.41 (m, 1H), 4.25-4.08 (m, 1H), 3.98-3.83 (m, 2H), 3.58 (td, 1H), 3.07-2.80 (m, 1H), 1.47 (s, 9H). | 3.31 min, [MH]+ = 448 | 2 |
| 1.47 | 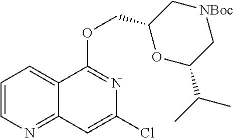 [SMILES: CC(C)[C@H]1CN(C(=O)OC(C)(C)C)C[C@@H](COc2nc(Cl)cc3ncccc23)O1] | 1H NMR (400 MHz, Methanol-d4) δ 9.01 (dd, 1H), 8.65 (ddd, 1H), 7.62 (dd, 1H), 7.50 (s, 1H), 4.83-4.72 (m, 2H), 4.69-4.44 (m, 1H), 4.34-4.23 (m, 1H), 3.87-3.38 (m, 4H), 1.89-1.68 (m, 1H), 1.57-1.30 (m, 9H), 0.98-0.82 (m, 6H) | 3.44 min, [MH]+ = 422 | 2 |
| 1.48 | 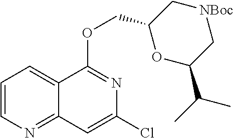 [SMILES: CC(C)[C@@H]1CN(C(=O)OC(C)(C)C)C[C@@H](COc2nc(Cl)cc3ncccc23)O1] | 1H NMR (400 MHz, Methanol-d4) δ 9.01 (dd, 1H), 8.64 (d, 1H), 7.62 (dd, 1H), 7.49 (d, 1H), 4.68-4.53 (m, 2H), 4.20-4.06 (m, 1H), 4.06-3.95 (m, 1H), 3.95-3.86 (m, 1H), 3.16 (ddd, 1H), 2.93-2.50 (m, 2H), 1.71 (h, 1H), 1.47 (s, 9H), 0.99-0.91 (m, 6H). | 3.56 min, [MH]+ = 422 | 2 |
| 1.49 | 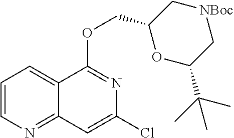 [SMILES: CC(C)(C)OC(=O)N1C[C@@H](COc2nc(Cl)cc3ncccc23)O[C@@H](C(C)(C)C)C1] | 1H NMR (400 MHz, Methanol-d4) δ 9.01 (dd, 1H), 8.66 (d, 1H), 7.62 (dd, 1H), 7.50 (s, 1H), 4.71-4.53 (m, 1H), 4.45-4.28 (m, 1H), 4.12- 3.94 (m, 2H), 3.54-3.42 (m, 1H), 3.28-3.11 (m, 2H), 2.97-2.66 (m, 1H), 1.62-1.25 (m, 9H), 1.00-0.71 (m, 9H). | 3.02 min, [MH]+ = 436 | Meth- od 4 - Col- umn 7 |
| 1.50 | 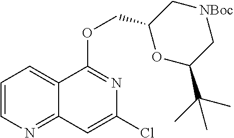 [SMILES: CC(C)(C)OC(=O)N1C[C@@H](COc2nc(Cl)cc3ncccc23)O[C@H](C(C)(C)C)C1] | 1H NMR (400 MHz, Methanol-d4) δ 8.94 (dd, 1H), 8.56 (d, 1H), 7.55 (dd, 1H), 7.42 (s, 1H), 4.64-4.42 (m, 2H), 4.11-3.98 (m, 1H), 3.98-3.88 (m, 1H), 3.88-3.77 (m, 1H), 3.03 (dd, 1H), 2.89-2.41 (m, 2H), 1.40 (s, 9H), 0.84 (s, 9H). | 3.16 mins, [MH]+ =436 | Meth- od 4- Col- umn 7 |
| 1.51 | 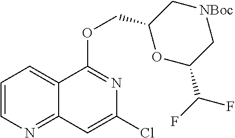 [SMILES: CC(C)(C)OC(=O)N1C[C@@H](COc2nc(Cl)cc3ncccc23)O[C@@H](C(F)F)C1] | 1H NMR (400 MHz, Methanol-d4) δ 9.02 (dd, 1H), 8.70-8.60 (m, 1H), 7.63 (dd, 1H), 7.51 (d, 1H), 5.94 (t, 1H), 4.78 (dd, 1H), 4.71-4.53 (m, 1H), 4.44-4.34 (m, 1H), 4.18-4.02 (m, 1H), 3.89-3.38 (m, 4H), 1.44 (s, 9H). | 3.13 min, [MH]+ = 430 | 2 |
| 1.52 | 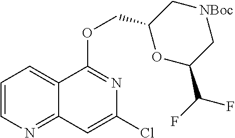 [SMILES: CC(C)(C)OC(=O)N1C[C@@H](COc2nc(Cl)cc3ncccc23)O[C@H](C(F)F)C1] | 1H NMR (400 MHz, Methanol-d4) δ 9.01 (dd, 1H), 8.64 (d, 1H), 7.62 (dd, 1H), 7.49 (d, 1H), 5.89 (td, 1H), 4.69-4.56 (m, 2H), 4.23-4.12 (m, 1H), 4.12-3.98 (m, 2H), 3.90-3.75 (m, 1H), 3.10-2.74 (m, 2H), 1.48 (s, 9H). | 3.17 min, [MH]+ = 430 | 2 |
| 1.53 | 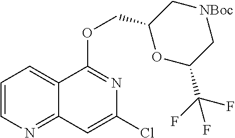 [SMILES: CC(C)(C)OC(=O)N1C[C@@H](COc2nc(Cl)cc3ncccc23)O[C@@H](C(F)(F)F)C1] | 1H NMR (400 MHz, Chloroform-d) δ 9.02 (dd, 1H), 8.53-8.47 (m, 1H), 7.56 (s, 1H), 7.45 (dd, 1H), 4.73 (dd, 1H), 4.68-4.51 (m, 1H), 4.50-4.41 (m, 1H), 4.32-4.13 (m, 1H), 3.99-3.30 (m, 4H), 1.51-1.41 (m, 9H). | 3.25 min, [MH]+ = 448 | 2 |
| 1.54 | 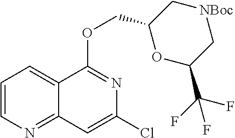 [SMILES: CC(C)(C)OC(=O)N1C[C@@H](COc2nc(Cl)cc3ncccc23)O[C@H](C(F)(F)F)C1] | 1H NMR (400 MHz, Chloroform-d) δ 9.02 (dd, 1H), 8.50 (ddd, 1H), 7.56 (d, 1H), 7.46 (dd, 1H), 4.76-4.54 (m, 2H), 4.35-4.08 (m, 2H), 4.07-3.93 (m, 2H), 3.04-2.74 (m, 2H), 1.49 (s, 9H). | 3.31 min, [MH]+ = 448 | 2 |
| 1.55 | 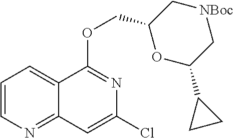 [SMILES: CC(C)(C)OC(=O)N1C[C@@H](COc2nc(Cl)cc3ncccc23)O[C@@H](C2CC2)C1] | 1H NMR (400 MHz, Methanol-d4) δ 9.02 (dd, 1H), 8.67 (d, 1H), 7.63 (dd, 1H), 7.50 (s, 1H), 4.88-4.70 (m, 2H), 4.67-4.45 (m, 1H), 4.44-4.30 (m, 1H), 3.90-3.51 (m, 3H), 3.18-3.05 (m, 1H), 1.52-1.32 (m, 9H), 0.92-0.80 (m, 1H), 0.57-0.34 (m, 2H), 0.33-0.09 (m, 2H). | 2.83 min, [MH]+ = 420 | Meth- od 4- Col- umn 7 |
| 1.56 | 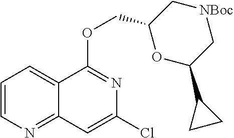 [SMILES: CC(C)(C)OC(=O)N1C[C@@H](COc2nc(Cl)cc3ncccc23)O[C@H](C2CC2)C1] | 1H NMR (400 MHz, Methanol-d4) δ 9.02 (dd, 1H), 8.66 (d, 1H), 7.63 (dd, 1H), 7.50 (s, 1H), 4.72-4.50 (m, 2H), 4.20-3.97 (m, 2H), 3.95-3.83 (m, 1H), 3.02-2.61 (m, 3H), 1.47 (s, 9H), 1.01-0.75 (m, 1H), 0.95-0.46 (m, 2H), 0.46-0.35 (m, 1H), 0.35-0.23 (m, 1H). | 2.92 min, [MH]+ = 420 | Meth- od 4- Col- umn 7 |
| 1.57 | 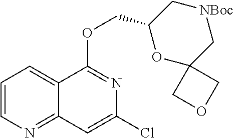 [SMILES: CC(C)(C)OC(=O)N1C[C@@H](COc2nc(Cl)cc3ncccc23)OC2(COC2)C1] | 1H NMR (400 MHz, Chloroform-d) δ 9.02 (dd, 1H), 8.54-8.48 (m, 1H), 7.56 (d, 1H), 7.46 (dd, 1H), 4.71-4.48 (m, 5H), 4.48-4.35 (m, 2H), 4.26- 3.99 (m, 1H), 3.95-3.86 (m, 1H), 3.07-2.92 (m, 1H), 2.92-2.75 (m, 1H), 1.49 (s, 9H). | 2.90 min, [MH]+ = 422 | 2 |
| 1.58 | 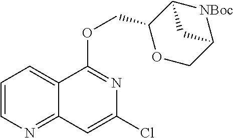 [SMILES: CC(C)(C)OC(=O)N1[C@H]2CO[C@H](COc3nc(Cl)cc4ncccc34)[C@@H]1C2] | 1H NMR (400 MHz, Methanol-d4) δ 9.01 (dd, 1H), 8.62 (s, 1H), 7.61 (ddd, 1H), 7.48 (d, 1H), 4.69-4.58 (m, 2H), 4.57-4.49 (m, 1H), 4.31- 4.18 (m, 2H), 4.18-4.11 (m, 1H), 3.88 (d, 1H), 2.63-2.54 (m, 1H), 2.02 (d, 1H), 1.45 (s, 9H). | 3.03 min, [MH]+ = 392 | 2 |
| 1.59 | 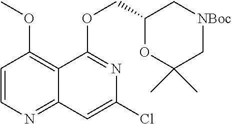 [SMILES: COc1ccnc2cc(Cl)nc(OC[C@@H]3CN(C(=O)OC(C)(C)C)CC(C)(C)O3)c12] | 1H NMR (400 MHz, Methanol-d4) δ 8.73 (d, 1H), 7.34 (s, 1H), 7.09 (d, 1H), 4.50 (dd, 1H), 4.43-4.22 (m, 2H), 4.19-4.10 (m, 4H), 3.85-3.80 (m, 1H), 3.01-2.64 (m, 2H), 1.49 (s, 9H), 1.28 (s, 3H), 1.23 (s, 3H). | 3.07 min, [MH]+ = 438 | 2 |
| 1.60 | 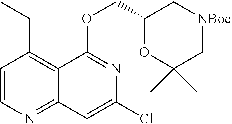 [SMILES: CCc1ccnc2cc(Cl)nc(OC[C@@H]3CN(C(=O)OC(C)(C)C)CC(C)(C)O3)c12] | 1H NMR (400 MHz, Methanol-d4) δ 8.81 (d, 1H), 7.45 (s, 1H), 7.44 (dd, 1H), 4.54-4.44 (m, 2H), 4.27-4.19 (m, 1H), 4.20-4.09 (m, 1H), 3.86-3.78 (m, 1H), 3.43-3.33 (m, 2H), 2.96-2.63 (m, 2H), 1.49 (s, 9H), 1.37 (t, 3H), 1.28 (s, 3H), 1.24 (s, 3H). | 3.55 min, [MH]+ = 436 | 2 |

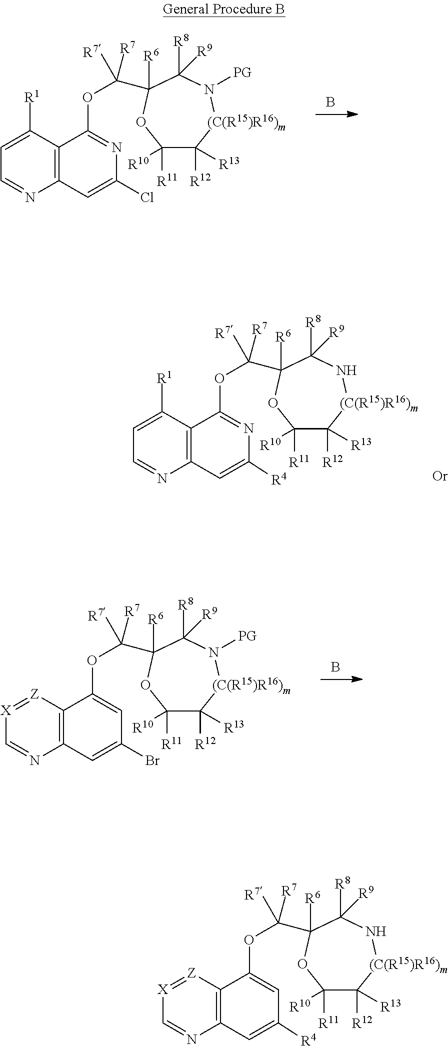

To a microwave vial was added tetrakis(triphenylphosphine)palladium (0.1 eq), sodium carbonate (3.0 eq), the appropriate boronic acid/ester (1.2-1.5 eq) and the appropriately substituted heteroaryl halide (1.0 eq). The vial was evacuated and back filled with nitrogen. This was repeated twice more before addition of a degassed solution of 1,4-dioxane/water (0.1-0.4 M in a 10:1 ratio). The reaction was heated at 135° C. under microwave irradiation for between 30 minutes to 1 hour. The cooled reaction mixture was partitioned between water and ethyl acetate. The aqueous layer was further extracted with ethyl acetate and the combined organics were washed with brine, dried over anhydrous sodium/magnesium sulfate and concentrated. The residue was either purified by standard purification method 1, 2 or 3 or taken to the next step as the crude product where the protecting group was removed using either of the following conditions:

### Boc Deprotection

Conditions 1: To a solution of protected intermediate (1.0 eq) in dichloromethane (0.05-0.2 M) was added trifluoroacetic acid (6-60 eq). The reaction mixture was stirred at room temperature for 1-24 hours. On consumption of starting materials, the reaction mixture was purified using one of the standard purification methods.

Conditions 2: A solution of intermediate (1.0 eg) in 1,4-dioxane/water (1:3 ratio, 0.05-0.2 M) was heated at 140-170° C. by microwave irradiation for 1-2 hours. The solvents were removed under reduced pressure and the reaction mixture was purified using one of the standard purification methods.

### Cbz Deprotection

To the protected intermediate (1.0 eq) was added hydrobromic acid solution (30% wt in acetic acid, 25-50 eq) and the ensuing solution was stirred at room temperature for 10 minutes. After this time, the reaction mixture was treated with hydrochloric acid (1 M aqueous solution) and the resulting mixture was extracted with dichloromethane. The aqueous phase was neutralised with sodium hydroxide and the products were extracted with ethyl acetate. The combined organic extracts were washed with brine, dried anhydrous sodium/magnesium sulfate and concentrated to give the deprotected product.

TABLE 2
Analytical data for naphthyridines synthesised by general method B:

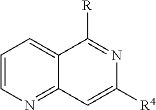

| | | | | LC MS RT, | LC MS | |
|---|---|---|---|---|---|---|
| No | R | R⁴ | 1H NMR | m/z | Method | Name |
| 2.1 | 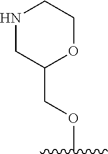 [SMILES: *OCC1CNCCO1] | 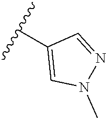 [SMILES: *c1cnn(C)c1] | 1H NMR (400 MHz, Methanol-d4) δ 8.90 (dd, 1H), 8.56 (ddd, 1H), 8.21 (s, 1H), 8.06 (d, 1H), 7.55 (d, 1H), 7.48 (dd, 1H), 4.58 (d, 2H), 4.02 (dtd, 1H), 3.95-3.90 (m, 4H), 3.69 (ddd, 1H), 3.08 (dd, 1H), 2.94-2.73 (m, 3H). | 1.51 min, [MH]+ = 326 | 1 | 7-(1-methyl-1H-pyrazol-4- yl)-5-[(morpholin-2- yl)methoxy]-1,6- naphthyridine |
| 2.2 | 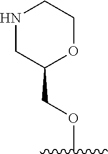 [SMILES: *OC[C@H]1CNCCO1] | 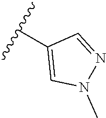 [SMILES: *c1cnn(C)c1] | 1H NMR (400 MHz, Methanol-d4) δ 8.92 (dd, 1H), 8.59 (ddd, 1H), 8.24 (s, 1H), 8.08 (d, 1H), 7.58 (d, 1H), 7.50 (dd, 1H), 4.60 (d, 2H), 4.02 (dtd, 1H), 3.96 (s, 3H), 3.94-3.88 (m, 1H), 3.74-3.63 (m, 1H), 3.11-3.03 (m, 1H), 2.92- 2.74 (m, 3H). | 1.51 min, [MH]+ = 326 | 1 | 7-(1-methyl-1H-pyrazol-4- yl)-5-{[(2R)-morpholin-2- yl]methoxy}-1,6- naphthyridine |
| 2.3 | 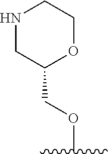 [SMILES: *OC[C@@H]1CNCCO1] | 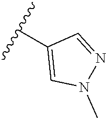 [SMILES: *c1cnn(C)c1] | 1H NMR (400 MHz, Methanol-d4) δ 8.92 (dd, 1H), 8.60 (ddd, 1H), 8.24 (s, 1H), 8.08 (d, 1H), 7.59 (d, 1H), 7.50 (dd, 1H), 4.60 (d, 2H), 4.06-3.99 (m, 1H), 3.96 (s, 3H), 3.92 (ddd, 1H), 3.68 (ddd, 1H), 3.07 (dd, 1H), 2.91-2.74 (m, 3H). | 1.45 min, [MH]+ = 326 | 2 | 7-(1-methyl-1H-pyrazol-4- yl)-5-{[(2S)-morpholin-2- yl]methoxy}-1,6- naphthyridine |
| 2.4 | 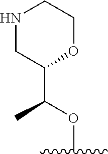 [SMILES: *O[C@@H](C)[C@@H]1CNCCO1] | 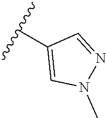 [SMILES: *c1cnn(C)c1] | 1H NMR (400 MHz, Methanol-d4) δ 8.91 (ddd, 1H), 8.64- 8.55 (m, 1H), 8.23 (s, 1H), 8.09-8.03 (m, 1H), 7.59-7.54 (m, 1H), 7.49 (ddd, 1H), 5.79-5.57 (m, 1H), 3.99-3.87 (m, 4H), 3.80 (ddd, 1H), 3.66 (ddd, 1H), 2.96 (dd, 1H), 2.89 2.69 (m, 3H), 1.46 (d, 3H). | 1.65 min, [MH]+ = 340 | 1 | 7-(1-methyl-1H-pyrazol-4- yl)-5-[(1S)-1-[(2S)- morpholin-2-yl]ethoxy]-1,6- naphthyridine |
| 2.5 | 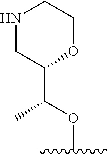 [SMILES: *O[C@H](C)[C@@H]1CNCCO1] | 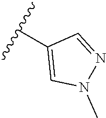 [SMILES: *c1cnn(C)c1] | 1H NMR (400 MHz, Methanol-d4) δ 8.91 (dd, 1H), 8.57 (ddd, 1H), 8.23 (app t, 1H), 8.07 (d, 1H), 7.57 (d, 1H), 7.49 (dd, 1H), 5.61-5.52 (m, 1H), 3.96 (s, 3H), 3.95-3.88 (m, 1H), 3.77 (ddd, 1H), 3.65 (ddd, 1H), 3.10 (dd, 1H), 2.88- 2.72 (m, 3H), 1.49 (d, 3H). | 1.54 min, [MH]+ = 340 | 2 | 7-(1-methyl-1H-pyrazol-4- yl)-5-[(1R)-1-[(2S)- morpholin-2-yl]ethoxy]-1,6- naphthyridine |
| 2.6 | 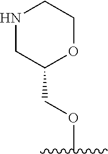 [SMILES: *OC[C@@H]1CNCCO1] | 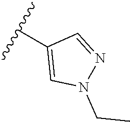 [SMILES: *c1cnn(CC)c1] | 1H NMR (400 MHz, Methanol-d4) δ 8.91 (dd, 1H), 8.59 (ddd, 1H), 8.28 (d, 1H), 8.09 (d, 1H), 7.58 (d, 1H), 7.49 (dd, 1H), 4.61 (d, 2H), 4.25 (q, 2H), 4.11-4.00 (m, 1H), 3.94 (d, 1H), 3.77-3.66 (m, 1H), 3.18-3.09 (m, 1H), 2.96 2.78 (m, 3H), 1.51 (t, 3H). | 1.62 min, [MH]+ = 340 | 1 | 7-(1-ethyl-1H-pyrazol-4-yl)- 5-{[(2S)-morpholin-2- yl]methoxy}-1,6- naphthyridine |
| 2.7 | 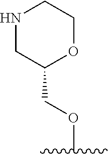 [SMILES: *OC[C@@H]1CNCCO1] | 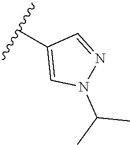 [SMILES: *c1cnn(C(C)C)c1] | 1H NMR (400 MHz, Methanol-d4) δ 8.92 (dd, 1H), 8.60 (ddd, 1H), 8.31 (d, 1H), 8.10 (d, 1H), 7.60 (d, 1H), 7.50 (dd, 1H), 4.67-4.52 (m, 3H), 4.08-4.02 (m, 1H), 3.98- 3.90 (m, 1H), 3.71 (ddd, 1H), 3.12 (dd, 1H), 2.96-2.78 (m, 3H), 1.55 (d, 6H) | 1.69 min, [MH]+ = 354 | 1 | 5-{[(2S)-morpholin-2- yl]methoxy}-7-[1-(propan-2- yl)-1H-pyrazol-4-yl]-1,6- naphthyridine |
| 2.8 | 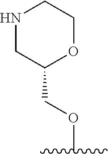 [SMILES: *OC[C@@H]1CNCCO1] | 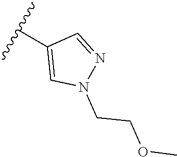 [SMILES: *c1cnn(CCOC)c1] | 1H NMR (400 MHz, Methanol-d4) δ 8.91 (dd, 1H), 8.58 (ddd, 1H), 8.26 (d, 1H), 8.10 (d, 1H), 7.58 (d, 1H), 7.49 (dd, 1H), 4.60 (d, 2H), 4.40-4.32 (m, 2H), 4.05-3.99 (m, 1H), 3.95-3.90 (m, 1H), 3.78 (t, 2H), 3.69 (ddd, 1H), 3.34 (s, 3H), 3.08 (dd, 1H), 2.92-2.75 (m, 3H). | 1.56 min, [MH]+ = 370 | 1 | 7-[1-(2-methoxyethyl)-1H- pyrazol-4-yl]-5-{[(2S)- morpholin-2-yl]methoxy}- 1,6-naphthyridine |
| 2.9 | 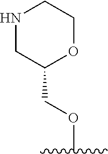 [SMILES: *OC[C@@H]1CNCCO1] | 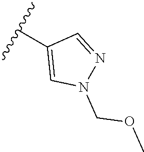 [SMILES: *c1cnn(COC)c1] | 1H NMR (400 MHz, Methanol-d4) δ 8.92 (dd, 1H), 8.59 (ddd, 1H), 8.44 (d, 1H), 8.18 (d, 1H), 7.63 (d, 1H), 7.51 (dd, 1H), 5.46 (s, 2H), 4.61 (d, 2H), 4.07-4.01 (m, 1H), 3.98-3.89 (m, 1H), 3.70 (ddd, 1H), 3.36 (s, 3H), 3.10 (dd, 1H), 2.93-2.76 (m, 3H) | 1.54 min, [MH]+ = 356 | 1 | 7-[1-(methoxymethyl)-1H- pyrazol-4-yl]-5-{[(2S)- morpholin-2-yl]methoxy}- 1,6-naphthyridine |
| 2.10 | 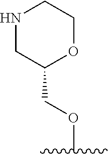 [SMILES: *OC[C@@H]1CNCCO1] | 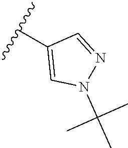 [SMILES: *c1cnn(C(C)(C)C)c1] | 1H NMR (400 MHz, Methanol-d4) δ 8.92 (dd, 1H), 8.60 (ddd, 1H), 8.36 (d, 1H), 8.11 (d, 1H), 7.61 (d, 1H), 7.50 (dd, 1H), 4.62 (d, 2H), 4.06-4.00 (m, 1H), 3.96-3.89 (m, 1H), 3.75-3.64 (m, 1H), 3.09 (dd, 1H), 2.93-2.75 (m, 3H), 1.65 (s, 9H). | 1.66 min, [MH]+ = 368 | 2 | 7-(1-tert-butyl-1H-pyrazol- 4-yl)-5-{[(2S)-morpholin-2- yl]methoxy}-1,6- naphthyridine |
| 2.11 | 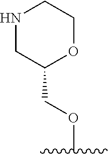 [SMILES: *OC[C@@H]1CNCCO1] | 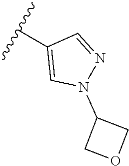 [SMILES: *c1cnn(C2COC2)c1] | 1H NMR (400 MHz, Methanol-d4) δ 8.93 (dd, 1H), 8.60 (ddd, 1H), 8.42 (d, 1H), 8.22 (s, 1H), 7.63 (d, 1H), 7.51 (dd, 1H), 5.69-5.59 (m, 1H), 5.15-5.04 (m, 4H), 4.62 (d, 2H), 4.07-3.99 (m, 1H), 3.96-3.89 (m, 1H), 3.74-3.64 (m, 1H), 3.09 (dd, 1H), 2.92-2.75 (m, 3H). | 1.48 min, [MH]+ = 368 | 2 | 5-{[(2S)-morpholin-2- yl]methoxy}-7-[1-(oxetan-3- yl)-1H-pyrazol-4-yl]-1,6- naphthyridine |
| 2.12 | 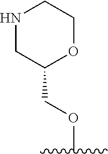 [SMILES: *OC[C@@H]1CNCCO1] | 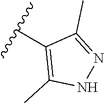 [SMILES: *c1c(C)n[nH]c1C] | 1H NMR (400 MHz, Methanol-d4) δ 8.92 (dd, 1H), 8.65- 8.57 (m, 1H), 7.52 (dd, 1H), 7.45 (d, 1H), 4.63-4.47 (m, 2H), 4.07-3.96 (m, 1H), 3.92 (d, 1H), 3.75-3.62 (m, 1H), 3.08 (d, 1H), 2.94-2.73 (m, 3H), 2.52 (s, 6H). | 1.45 min, [MH]+ = 340 | 2 | 7-(3,5-dimethyl-1H-pyrazol- 4-yl)-5-{[(2S)-morpholin-2- yl]methoxy}-1,6- naphthyridine |
| 2.13 | 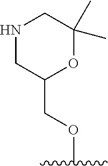 [SMILES: *OCC1CNCC(C)(C)O1] | 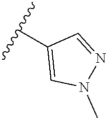 [SMILES: *c1cnn(C)c1] | 1H NMR (400 MHz, Methanol-d4) δ 8.91 (dd, 1H), 8.57 (ddd, 1H), 8.23 (s, 1H), 8.08 (d, 1H), 7.58 (d, 1H), 7.50 (dd, 1H), 4.60-4.47 (m, 2H), 4.26 (ddt, 1H), 3.96 (s, 3H), 3.08 (ddd, 1H), 2.75-2.54 (m, 3H), 1.36 (s, 3H), 1.19 (s, 3H). | 1.60 min, [MH]+ = 354 | 2 | 5-[(6,6-dimethylmorpholin- yl)methoxy]-7-(1-methyl- 1H-pyrazol-4-yl)-1,6- naphthyridine |
| 2.14 | 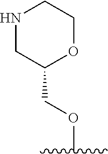 [SMILES: *OC[C@@H]1CNCCO1] | 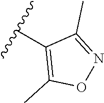 [SMILES: *c1c(C)noc1C] | 1H NMR (400 MHz, Chloroform-d) δ 9.01 (dd, 1H), 8.56 (ddd, 1H), 7.52 (d, 1H), 7.45 (dd, 1H), 4.58 (dd, 1H), 4.51 (dd, 1H), 4.02 (dddd, 1H), 3.99-3.94 (m, 1H), 3.77-3.68 (m, 1H), 3.13 (dd, 1H), 2.97 (ddd, 1H), 2.93-2.83 (m, 2H), 2.69 (s, 3H), 2.54 (s, 3H) | 1.62 min, [MH]+ = 341 | 2 | 7-(3,5-dimethyl-1,2-oxazol- 4-yl)-5-{[(2S)-morpholin-2- yl]methoxy}-1,6- naphthyridine |
| 2.15 | 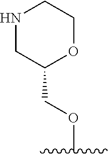 [SMILES: *OC[C@@H]1CNCCO1] | 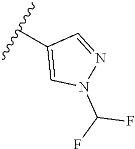 [SMILES: *c1cnn(C(F)F)c1] | 1H NMR (400 MHz, Chloroform-d) δ 8.98 (dd, 1H), 8.53 (ddd, 1H), 8.38 (d, 1H), 8.18 (d, 1H), 7.67 (d, 1H), 7.40 (dd, 1H), 7.25 (t, 1H), 4.63 (dd, 1H), 4.57 (dd, 1H), 4.03 (dddd, 1H), 3.98 (ddd, 1H), 3.77-3.68 (m, 1H), 3.14 (dd, 1H), 2.97 (ddd, 1H), 2.92-2.83 (m, 2H) | 1.61 min, [MH]+ = 362 | 2 | 7-[1-(difluoromethyl)-1H- pyrazol-4-yl]-5-{[(2S)- morpholin-2-yl]methoxy}- 1,6-naphthyridine |
| 2.16 | 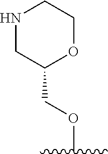 [SMILES: *OC[C@@H]1CNCCO1] | 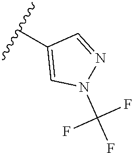 [SMILES: *c1cnn(C(F)(F)F)c1] | 1H NMR (400 MHz, Chloroform-d) δ 9.00 (dd, 1H), 8.53 (ddd, 1H), 8.36 (s, 1H), 8.29-8.26 (m, 1H), 7.69 (d, 1H), 7.42 (dd, 1H), 4.62 (dd, 1H), 4.56 (dd, 1H), 4.04 (dddd, 1H), 3.98 (ddd, 1H), 3.79-3.69 (m, 1H), 3.14 (dd, 1H), 2.98 (ddd, 1H), 2.93-2.84 (m, 2H). | 1.74 min, [MH]+ = 380 | 2 | 5-{[(2S)-morpholin-2- yl]methoxy}-7-[1- (trifluoromethyl)-1H- pyrazol-4-yl]-1,6- naphthyridine |
| 2.17 | 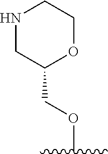 [SMILES: *OC[C@@H]1CNCCO1] | 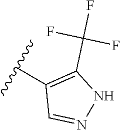 [SMILES: *c1cn[nH]c1C(F)(F)F] | 1H NMR (400 MHz, Chloroform-d) δ 9.02 (dd, 1H), 8.57 (ddd, 1H), 8.15 (d, 1H), 7.74 (d, 1H), 7.44 (dd, 1H), 4.64 (dd, 1H), 4.57 (dd, 1H), 4.03 (dddd, 1H), 4.02-3.94 (m, 1H), 3.77-3.66 (m, 1H), 3.12 (dd, 1H), 2.98 (ddd, 1H), 2.93-2.82 (m, 2H). | 1.61 min, [MH]+ = 380 | 2 | 5-{[(2S)-morpholin-2- yl]methoxy}-7-[5- (trifluoromethyl)-1H- pyrazol-4-yl]-1,6- naphthyridine |
| 2.18 | 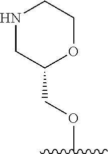 [SMILES: *OC[C@@H]1CNCCO1] | 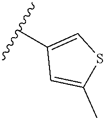 [SMILES: *c1csc(C)c1] | 1H NMR (600 MHz, Methanol-d4) δ 8.90 (dd, 1H), 8.54 (dd, 1H), 7.87 (d, 1H), 7.60 (s, 1H), 7.49 (dd, 1H), 7.38 (s, 1H), 4.57 (d, 2H), 4.04-3.97 (m, 1H), 3.92 (dd, 1H), 3.72- 3.64 (m, 1H), 3.06 (dd, 1H), 2.90-2.74 (m, 3H), 2.53 (s, 3H). | 1.81 min, [MH]+ = 342 | 2 | 7-(5-methylthiophen-3-yl)-5- {[(2S)-morpholin-2- yl]methoxy}-1,6- naphthyridine |
| 2.19 | 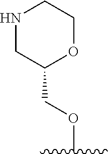 [SMILES: *OC[C@@H]1CNCCO1] | 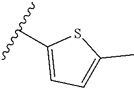 [SMILES: *c1ccc(C)s1] | 1H NMR (600 MHz, Methanol-d4) δ 8.85 (dd, 1H), 8.47 (dd, 1H), 7.53 (s, 1H), 7.50 (d, 1H), 7.42 (dd, 1H), 6.77 (d, 1H), 4.54-4.45 (m, 2H), 4.02-3.95 (m, 1H), 3.91 (dd, 1H), 3.72-3.63 (m, 1H), 3.06 (dd, 1H), 2.90-2.78 (m, 2H), 2.75 (dd, 1H), 2.49 (s, 3H). | 1.81 min, [MH]+ = 342 | 2 | 7-(5-methylthiophen-2-yl)-5- {[(2S)-morpholin-2- yl]methoxy}-1,6- naphthyridine |
| 2.20 | 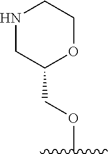 [SMILES: *OC[C@@H]1CNCCO1] | 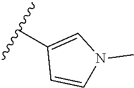 [SMILES: *c1ccn(C)c1] | 1H NMR (400 MHz, Methanol-d4) δ 8.84 (ddd, 1H), 8.57- 8.51 (m, 1H), 7.45 (d, 1H), 7.44-7.38 (m, 2H), 6.70 (app t, 1H), 6.65 (ddd, 1H), 4.61-4.56 (m, 2H), 4.06-3.97 (m, 1H), 3.95-3.88 (m, 1H), 3.72 (s, 3H), 3.71-3.63 (m, 1H), 3.07 (dd, 1H), 2.91-2.73 (m, 3H). | 1.58 min, [MH]+ = 325 | 2 | 7-(1-methyl-1H-pyrrol-3-yl)- 5-{[(2S)-morpholin-2- yl]methoxy}-1,6- naphthyridine |
| 2.21 | 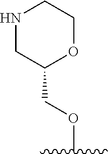 [SMILES: *OC[C@@H]1CNCCO1] | 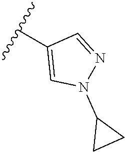 [SMILES: *c1cnn(C2CC2)c1] | 1H NMR (400 MHz, Methanol-d4) δ 8.89 (dd, 1H), 8.55 (ddd, 1H), 8.27 (s, 1H), 8.04 (d, 1H), 7.54 (d, 1H), 7.47 (dd, 1H), 4.66-4.44 (m, 2H), 4.06-3.97 (m, 1H), 3.93 (ddd, 1H), 3.78-3.63 (m, 2H), 3.07 (dd, 1H), 2.92-2.74 (m, 3H), 1.22-1.14 (m, 2H), 1.14-1.05 (m, 2H). | 1.58 min, [MH]+ = 352 | 2 | 7-(1-cyclopropyl-1H- pyrazol-4-yl)-5-{[(2S)- morpholin-2-yl]methoxy}- 1,6-naphthyridine |
| 2.22 | 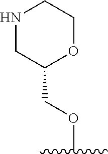 [SMILES: *OC[C@@H]1CNCCO1] | 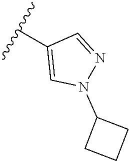 [SMILES: *c1cnn(C2CCC2)c1] | 1H NMR (600 MHz, Methanol-d4) δ 8.89 (d, 1H), 8.53 (d, 1H), 8.29 (s, 1H), 8.08 (s, 1H), 7.54 (s, 1H), 7.46 (dd, 1H), 4.93-4.84 (m, 1H), 4.60-4.54 (m, 2H), 4.04-3.97 (m, 1H), 3.93 (dd, 1H), 3.73-3.65 (m, 1H), 3.07 (dd, 1H), 2.92- 2.74 (m, 3H), 2.68-2.56 (m, 2H), 2.55-2.45 (m, 2H), 1.97-1.87 (m, 2H). | 1.67 min, [MH]+ = 366 | 2 | 7-(1-cyclobutyl-1H-pyrazol- 4-yl)-5-{[(2S)-morpholin-2- yl]methoxy}-1,6- naphthyridine |
| 2.23 | 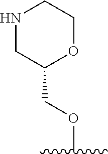 [SMILES: *OC[C@@H]1CNCCO1] | 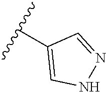 [SMILES: *c1cn[nH]c1] | 1H NMR (600 MHz, Methanol-d4) δ 8.91 (dd, 1H), 8.57 (dd, 1H), 8.22 (br s, 2H), 7.60 (s, 1H), 7.49 (dd, 1H), 4.59 (d, 2H), 4.05-3.99 (m, 1H), 3.93 (dd, 1H), 3.73-3.65 (m, 1H), 3.08 (dd, 1H), 2.91-2.75 (m, 3H). | 1.38 min, [MH]+ = 312 | 2 | 5-{[(2S)-morpholin-2- yl]methoxy}-7-(1H-pyrazol- 4-yl)-1,6-naphthyridine |
| 2.24 | 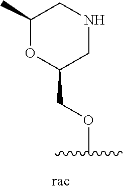 [SMILES: *OC[C@@H]1CNC[C@H](C)O1] | 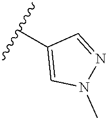 [SMILES: *c1cnn(C)c1] | 1H NMR (400 MHz, Methanol-d4) δ 8.91 (dd, 1H), 8.58 (d, 1H), 8.23 (s, 1H), 8.07 (s, 1H), 7.57 (s, 1H), 7.50 (dd, 1H), 4.67-4.49 (m, 2H), 4.12-3.99 (m, 1H), 3.96 (s, 3H), 3.76- 3.65 (m, 1H), 3.05 (d, 1H), 2.85 (d, 1H), 2.72-2.62 (m, 1H), 2.43 (dd, 1H), 1.16 (d, 3H). | 1.51 min, [MH]+ = 340 | 2 | 7-(1-methyl-1H-pyrazol-4- yl)-5-{[(rel-2S,6S)-6- methylmorpholin-2- yl]methoxy}-1,6- naphthyridine |
| 2.25 | 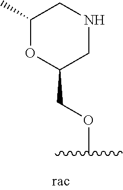 [SMILES: *OC[C@@H]1CNC[C@@H](C)O1] | 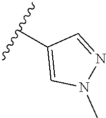 [SMILES: *c1cnn(C)c1] | 1H NMR (400 MHz, Methanol-d4) δ 8.91 (dd, 1H), 8.59 (ddd, 1H), 8.27 (app t, 1H), 8.10 (d, 1H), 7.58 (d, 1H), 7.49 (dd, 1H), 5.05 (dd, 1H), 4.65 (dd, 1H), 4.32-4.23 (m, 1H), 4.05 (ddt, 1H), 3.96 (s, 3H), 3.04 (dd, 1H), 2.97-2.88 (m, 2H), 2.54 (dd, 1H), 1.14 (d, 3H). | 1.50 min, [MH]+ = 340 | 2 | 7-(1-methyl-1H-pyrazol-4- yl)-5-{[(rel-2S,6R)-6- methylmorpholin-2- yl]methoxy}-1,6- naphthyridine |
| 2.26 | 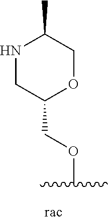 [SMILES: *OC[C@@H]1CN[C@@H](C)CO1] | 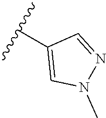 [SMILES: *c1cnn(C)c1] | 1H NMR (400 MHz, Methanol-d4) δ 8.92 (dd, 1H), 8.59 (ddd, 1H), 8.24 (s, 1H), 8.08 (d, 1H), 7.58 (d, 1H), 7.50 (dd, 1H), 4.66-4.54 (m, 2H), 4.00-3.92 (m, 4H), 3.87 (dd, 1H), 3.23 (dd, 1H), 3.10 (dd, 1H), 2.92-2.78 (m, 2H), 1.00 (d, 3H). | 1.50 min, [MH]+ = 340 | 2 | 7-(1-methyl-1H-pyrazol-4- yl)-5-{[(rel-2S,5S)-5- methylmorpholin-2- yl]methoxy}-1,6- naphthyridine |
| 2.27 | 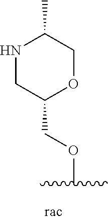 [SMILES: *OC[C@@H]1CN[C@H](C)CO1] | 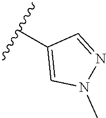 [SMILES: *c1cnn(C)c1] | 1H NMR (400 MHz, Methanol-d4) δ 8.92 (dd, 1H), 8.60 (ddd, 1H), 8.27 (d, 1H), 8.10 (d, 1H), 7.59 (d, 1H), 7.50 (dd, 1H), 4.88 (dd, 1H), 4.65 (dd, 1H), 4.12-4.04 (m, 1H), 3.96 (s, 3H), 3.73 (dd, 1H), 3.65 (dd, 1H), 3.11 (dd, 1H), 3.00-2.91 (m, 2H), 1.23 (d, 3H). | 1.47 min, [MH]+ = 340 | 2 | 7-(1-methyl-1H-pyrazol-4- yl)-5-{[(rel-2S,5R)-5- methylmorpholin-2- yl]methoxy}-1,6- naphthyridine |
| 2.28 | 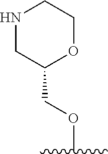 [SMILES: *OC[C@@H]1CNCCO1] | 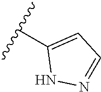 [SMILES: *c1ccn[nH]1] | 1H NMR (400 MHz, Methanol-d4) δ 8.97 (dd, 1H), 8.65 (ddd, 1H), 7.90 (br s, 1H), 7.69 (br s, 1H), 7.57 (dd, 1H), 6.97 (s, 1H), 4.68 (d, 2H), 4.05-4.00 (m, 1H), 3.93 (ddd, 1H), 3.69 (ddd, 1H), 3.08 (dd, 1H), 2.93-2.74 (m, 3H). | 1.51 min, [MNa]+ = 334 | 2 | 5-{[(2S)-morpholin-2- yl]methoxy}-7-(1H-pyrazol- 5-yl)-1,6-naphthyridine |
| 2.29 | 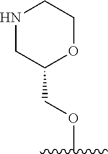 [SMILES: *OC[C@@H]1CNCCO1] | 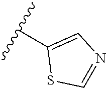 [SMILES: *c1cncs1] | 1H NMR (400 MHz, DMSO-d6) δ 9.17 (s, 1H), 9.08 (dd, 1H), 8.73 (s, 1H), 8.52 (dd, 1H), 8.06 (s, 1H), 7.62 (dd, 1H), 4.52 (d, 2H), 3.92 (ddd, 1H), 3.81 (d, 1H), 3.57-3.49 (m, 1H), 3.04 (d, 1H), 2.85-2.62 (m, 3H). | 1.53 min, [MH]+ = 329 | 2 | 5-{[(2S)-morpholin-2- yl]methoxy}-7-(1,3-thiazol- 5-yl)-1,6-naphthyridine |
| 2.30 | 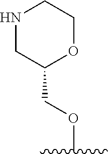 [SMILES: *OC[C@@H]1CNCCO1] | 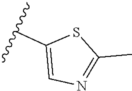 [SMILES: *c1cnc(C)s1] | 1H NMR (400 MHz, Methanol-d4) δ 8.98 (dd, 1H), 8.65- 8.59 (m, 1H), 8.27 (s, 1H), 7.76 (s, 1H), 7.57 (dd, 1H), 4.57 (d, 2H), 4.07-3.98 (m, 1H), 3.92 (d, 1H), 3.72-3.65 (m, 1H), 3.08 (d, 1H), 2.93-2.75 (m, 3H), 2.74 (s, 3H). | 1.63 min, [MH]+ = 343 | 2 | 7-(2-methyl-1,3-thiazol-5- yl)-5-{[(2S)-morpholin-2- yl]methoxy}-1,6- naphthyridine |
| 2.31 | 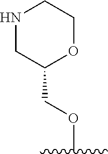 [SMILES: *OC[C@@H]1CNCCO1] | 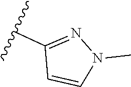 [SMILES: *c1ccn(C)n1] | 1H NMR (400 MHz, Methanol-d4) δ 8.99-8.91 (m, 1H), 8.63 (d, 1H), 7.95 (s, 1H), 7.65 (d, 1H), 7.55 (dd, 1H), 6.93 (d, 1H), 4.67 (d, 2H), 4.15-4.04 (m, 1H), 4.04-3.91 (m, 4H), 3.82-3.67 (m, 1H), 3.26-3.16 (m, 1H), 3.04-2.85 (m, 3H). | 1.48 min, [MH]+ = 326 | 2 | 7-(1-methyl-1H-pyrazol-3- yl)-5-{[(2S)-morpholin-2- yl]methoxy}-1,6- naphthyridine |
| 2.32 | 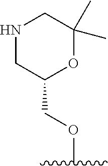 [SMILES: *OC[C@@H]1CNCC(C)(C)O1] | 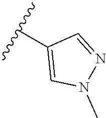 [SMILES: *c1cnn(C)c1] | 1H NMR (600 MHz, Methanol-d4) δ 8.94 (dd, 1H), 8.59 (d, 1H), 8.25 (s, 1H), 8.10 (s, 1H), 7.59 (s, 1H), 7.52 (dd, 1H), 4.58 (dd, 1H), 4.52 (dd, 1H), 4.29 (dtd, 1H), 3.99 (s, 3H), 3.11 (dd, 1H), 2.74 (d, 1H), 2.68-2.61 (m, 2H), 1.39 (s, 3H), 1.22 (s, 3H). | 1.60 min, [MH]+ = 354 | 2 | 5-{[(2S)-6,6- dimethylmorpholin-2- yl]methoxy}-7-(1-methyl- 1H-pyrazol-4-yl)-1,6- naphthyridine |
| 2.33 | 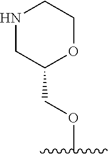 [SMILES: *OC[C@@H]1CNCCO1] | 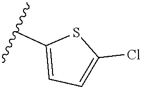 [SMILES: *c1ccc(Cl)s1] | 1H NMR (400 MHz, DMSO-d6) δ 9.06 (dd, 1H), 8.50 (ddd, 1H), 7.99 (d, 1H), 7.86 (d, 1H), 7.59 (dd, 1H), 7.23 (d, 1H), 4.50 (d, 2H), 3.94-3.83 (m, 1H), 3.83-3.74 (m, 1H), 3.50 (ddd, 1H), 2.98 (dd, 1H), 2.76-2.57 (m, 3H). | 1.91 min, [MH]+ = 362/364 | 2 | 7-(5-chlorothiophen-2-yl)-5- {[(2S)-morpholin-2- yl]methoxy}-1,6- naphthyridine |
| 2.34 | 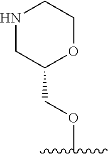 [SMILES: *OC[C@@H]1CNCCO1] | 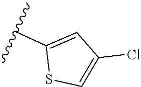 [SMILES: *c1cc(Cl)cs1] | 1H NMR (400 MHz, DMSO-d6) δ 9.07 (dd, 1H), 8.51 (ddd, 1H), 8.04 (d, 1H), 8.01 (d, 1H), 7.70 (d, 1H), 7.60 (dd, 1H), 4.52 (d, 2H), 3.95-3.85 (m, 1H), 3.84-3.74 (m, 1H), 3.51 (ddd, 1H), 3.01 (dd, 1H), 2.81-2.57 (m, 3H). | 1.91 min, [MH]+ = 362/364 | 2 | 7-(4-chlorothiophen-2-yl)-5- {[(2S)-morpholin-2- yl]methoxy}-1,6- naphthyridine |
| 2.35 | 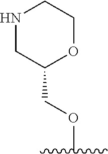 [SMILES: *OC[C@@H]1CNCCO1] | 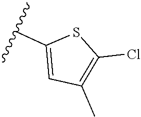 [SMILES: *c1cc(C)c(Cl)s1] | 1H NMR (600 MHz, DMSO-d6) δ 9.05 (dd, 1H), 8.49 (dd, 1H), 7.91 (s, 1H), 7.81 (s, 1H), 7.58 (dd, 1H), 4.49 (d, 2H), 3.92-3.83 (m, 1H), 3.78 (d, 1H), 3.53-3.46 (m, 1H), 2.98 (dd, 1H), 2.76-2.59 (m, 3H), 2.21 (s, 3H). | 2.00 min, [MH]+ = 376 | 2 | 7-(5-chloro-4- methylthiophen-2-yl)-5- {[(2S)-morpholin-2- yl]methoxy}-1,6- naphthyridine |
| 2.36 | 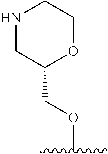 [SMILES: *OC[C@@H]1CNCCO1] | 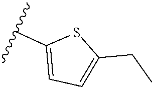 [SMILES: *c1ccc(CC)s1] | 1H NMR (400 MHz, Methanol-d4) δ 8.92 (dd, 1H), 8.58 (ddd, 1H), 7.67 (d, 1H), 7.61 (d, 1H), 7.50 (dd, 1H), 6.86 (app dt, 1H), 4.66-4.54 (m, 2H), 4.08-3.98 (m, 1H), 3.97- 3.87 (m, 1H), 3.68 (ddd, 1H), 3.09 (dd, 1H), 2.95-2.71 (m, 5H), 1.35 (t, 3H) | 1.95 min, [MH]+ = 356 | 2 | 7-(5-ethylthiophen-2-yl)-5- {[(2S)-morpholin-2- yl]methoxy}-1,6- naphthyridine |
| 2.37 | 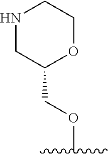 [SMILES: *OC[C@@H]1CNCCO1] | 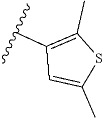 [SMILES: *c1cc(C)sc1C] | 1H NMR (400 MHz, Methanol-d4) δ 8.94 (dd, 1H), 8.60 (ddd, 1H), 7.59-7.49 (m, 2H), 7.17-7.11 (m, 1H), 4.63- 4.50 (m, 2H), 4.00 (dddd, 1H), 3.91 (ddd, 1H), 3.68 (ddd, 1H), 3.06 (dd, 1H), 2.91-2.73 (m, 3H), 2.71 (s, 3H), 2.47- 2.38 (m, 3H). | 1.90 min, [MH]+ = 356 | 2 | 7-(2,5-dimethylthiophen-3- yl)-5-{[(2S)-morpholin-2- yl]methoxy}-1,6- naphthyridine |
| 2.38 | 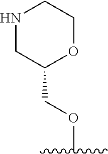 [SMILES: *OC[C@@H]1CNCCO1] | 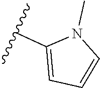 [SMILES: *c1cccn1C] | 1H NMR (400 MHz, Methanol-d4) δ 8.89 (dd, 1H), 8.56 (ddd, 1H), 7.57 (d, 1H), 7.46 (dd, 1H), 6.84-6.77 (m, 1H), 6.70 (dd, 1H), 6.13 (dd, 1H), 4.60-4.46 (m, 2H), 4.04 (s, 3H), 3.99 (dddd, 1H), 3.90 (ddd, 1H), 3.67 (ddd, 1H), 3.04 (dd, 1H), 2.91-2.71 (m, 3H). | 1.64 min, [MH]+ = 325 | 2 | 7-(1-methyl-1H-pyrrol-2-yl)- 5-{[(2S)-morpholin-2- yl]methoxy}-1,6- naphthyridine |
| 2.39 | 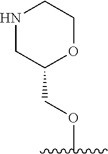 [SMILES: *OC[C@@H]1CNCCO1] | 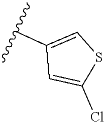 [SMILES: *c1csc(Cl)c1] | 1H NMR (400 MHz, Methanol-d4) δ 8.94 (dd, 1H), 8.58 (ddd, 1H), 7.98 (d, 1H), 7.65 (d, 1H), 7.63 (d, 1H), 7.53 (dd, 1H), 4.59 (d, 2H), 4.05-3.99 (m, 1H), 3.92 (ddd, 1H), 3.69 (ddd, 1H), 3.07 (dd, 1H), 2.93-2.74 (m, 3H). | 1.91 min, [MH]+ = 362 | 2 | 7-(5-chlorothiophen-3-yl)-5- {[(2S)-morpholin-2- yl]methoxy}-1,6- naphthyridine |
| 2.40 | 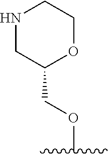 [SMILES: *OC[C@@H]1CNCCO1] | 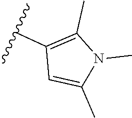 [SMILES: *c1cc(C)n(C)c1C] | 1H NMR (400 MHz, Methanol-d4) δ 8.83 (dd, 1H), 8.53 (ddd, 1H), 7.48-7.34 (m, 2H), 6.28 (d, 1H), 4.64-4.50 (m, 2H), 4.05-3.97 (m, 1H), 3.95-3.87 (m, 2H), 3.72- 3.63 (m, 1H), 3.47 (s, 3H), 3.06 (dd, 1H), 2.90-2.72 (m, 3H), 2.68 (s, 3H), 2.24 (s, 3H). | 1.63 min, [MH]+ = 353 | 2 | 5-{[(2S)-morpholin-2- yl]methoxy}-7-(1,2,5- trimethyl-1H-pyrrol-3-yl)- 1,6-naphthyridine |
| 2.41 | 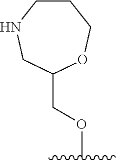 [SMILES: *OCC1CNCCCO1] | 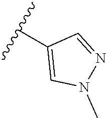 [SMILES: *c1cnn(C)c1] | 1H NMR (400 MHz, Methanol-d4) δ 8.90 (dd, 1H), 8.56 (ddd, 1H), 8.22 (s, 1H), 8.06 (d, 1H), 7.55 (d, 1H), 7.48 (dd, 1H), 4.61 (dd, 1H), 4.53 (dd, 1H), 4.14 (dddd, 1H), 4.04 (ddd, 1H), 3.95 (s, 3H), 3.79 (ddd, 1H), 3.23 (dd, 1H), 3.05- 2.97 (m, 1H), 2.96-2.84 (m, 2H), 2.02-1.81 (m, 2H). | 1.50 min, [MH]+ = 340 | 2 | 7-(1-methyl-1H-pyrazol-4- yl)-5-[(1,4-oxazepan-2- yl)methoxy]-1,6- naphthyridine |
| 2.42 | 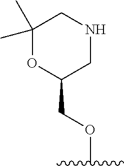 [SMILES: *OC[C@@H]1CNCC(C)(C)O1] | 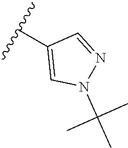 [SMILES: *c1cnn(C(C)(C)C)c1] | 1H NMR (400 MHz, Methanol-d4) δ 8.91 (dd, 1H), 8.57 (ddd, 1H), 8.35 (d, 1H), 8.11 (d, 1H), 7.60 (d, 1H), 7.49 (dd, 1H), 4.59 (dd, 1H), 4.48 (dd, 1H), 4.31-4.22 (m, 1H), 3.08 (ddd, 1H), 2.71 (dd, 1H), 2.67-2.56 (m, 2H), 1.65 (s, 9H), 1.36 (s, 3H), 1.19 (s, 3H). | 1.82 min, [MH]+ = 396 | 2 | 7-(1-tert-butyl-1H-pyrazol- 4-yl)-5-{[(2S)-6,6- dimethylmorpholin-2- yl]methoxy}-1,6- naphthyridine |
| 2.43 | 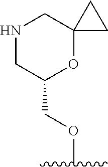 [SMILES: *OC[C@@H]1CNCC2(CC2)O1] | 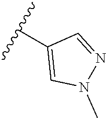 [SMILES: *c1cnn(C)c1] | 1H NMR (400 MHz, Methanol-d4) δ 8.92-8.86 (m, 1H), 8.56-8.47 (m, 1H), 8.18 (s, 1H), 8.04 (s, 1H), 7.57-7.51 (m, 1H), 7.47 (ddd, 1H), 4.62-4.48 (m, 2H), 4.21-4.11 (m, 1H), 3.95 (s, 3H), 3.30-3.25 (m, 1H), 3.13 (dd, 1H), 2.84 (ddd, 1H), 2.33 (d, 1H), 0.92-0.80 (m, 1H), 0.75 (d, 1H), 0.67-0.56 (m, 2H). | 1.68 min, [MH]+ = 352 | 2 | 7-(1-methyl-1H-pyrazol-4- yl)-5-{[(5S)-4-oxa-7- azaspiro[2.5]octan-5- yl]methoxy}-1,6- naphthyridine |
| 2.44 | 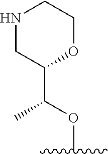 [SMILES: *O[C@H](C)[C@@H]1CNCCO1] | 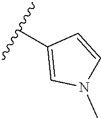 [SMILES: *c1ccn(C)c1] | 1H NMR (400 MHz, Methanol-d4) δ 8.83 (dd, 1H), 8.50 (ddd, 1H), 7.44-7.36 (m, 3H), 6.70 (app t, 1H), 6.64 (dd, 1H), 5.60-5.49 (m, 1H), 3.91 (ddd, 1H), 3.75 (ddd, 1H), 3.72 (s, 3H), 3.69-3.57 (m, 1H), 3.09 (dd, 1H), 2.88-2.67 (m, 3H), 1.48 (d, 3H). | 1.64 min, [MH]+ = 339 | 2 | 7-(1-methyl-1H-pyrrol-3-yl)- 5-[(1R)-1-[(2S)-morpholin- 2-yl]ethoxy]-1,6- naphthyridine |
| 2.45 | 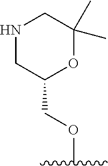 [SMILES: *OC[C@@H]1CNCC(C)(C)O1] | 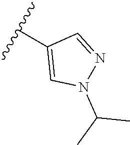 [SMILES: *c1cnn(C(C)C)c1] | 1H NMR (400 MHz, Methanol-d4) δ 8.92 (dd, 1H), 8.59 (ddd, 1H), 8.31 (d, 1H), 8.11 (d, 1H), 7.60 (d, 1H), 7.51 (dd, 1H), 4.67-4.54 (m, 2H), 4.50 (dd, 1H), 4.31-4.20 (m, 1H), 3.11-3.03 (m, 1H), 2.79-2.53 (m, 3H), 1.55 (d, 6H), 1.36 (s, 3H), 1.19 (s, 3H). | 1.74 min, [MH]+ = 382 | 2 | 5-{[(2S)-6,6- dimethylmorpholin-2- yl]methoxy}-7-[1-(propan-2- yl)-1H-pyrazol-4-yl]-1,6- naphthyridine |
| 2.46 | 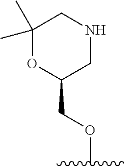 [SMILES: *OC[C@@H]1CNCC(C)(C)O1] | 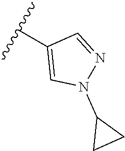 [SMILES: *c1cnn(C2CC2)c1] | 1H NMR (400 MHz, Methanol-d4) δ 8.92 (dd, 1H), 8.58 (ddd, 1H), 8.31 (app t, 1H), 8.08 (d, 1H), 7.60 (d, 1H), 7.51 (dd, 1H), 4.63-4.44 (m, 2H), 4.31-4.19 (m, 1H), 3.79- 3.68 (m, 1H), 3.08 (dd, 1H), 2.77-2.53 (m, 3H), 1.36 (s, 3H), 1.19 (s, 3H), 1.18-1.00 (m, 4H). | 1.71 min, [MH]+ = 380 | 2 | 7-(1-cyclopropyl-1H- pyrazol-4-yl)-5-{[(2S)-6,6- dimethylmorpholin-2- yl]methoxy}-1,6- naphthyridine |
| 2.47 | 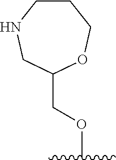 [SMILES: *OCC1CNCCCO1] | 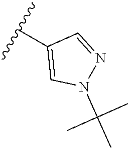 [SMILES: *c1cnn(C(C)(C)C)c1] | 1H NMR (400 MHz, Methanol-d4) δ 8.92 (dd, 1H), 8.60 (ddd, 1H), 8.36 (d, 1H), 8.12 (d, 1H), 7.61 (d, 1H), 7.50 (dd, 1H), 4.65 (dd, 1H), 4.56 (dd, 1H), 4.21-4.12 (m, 1H), 4.09-4.01 (m, 1H), 3.80 (ddd, 1H), 3.24 (dd, 1H), 3.02 (ddd, 1H), 2.97-2.87 (m, 2H), 2.01-1.84 (m, 2H), 1.65 (s, 9H). | 1.73 min, [MH]+ = 382 | 2 | 7-(1-tert-butyl-1H-pyrazol- 4-yl)-5-[(1,4-oxazepan-2- yl)methoxy]-1,6- naphthyridine |
| 2.48 | 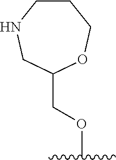 [SMILES: *OCC1CNCCCO1] | 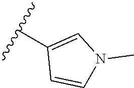 [SMILES: *c1ccn(C)c1] | 1H NMR (400 MHz, Methanol-d4) δ 8.84 (dd, 1H), 8.54 (ddd, 1H), 7.45 (d, 1H), 7.44-7.39 (m, 2H), 6.70 (app t, 1H), 6.65 (dd, 1H), 4.61 (dd, 1H), 4.53 (dd, 1H), 4.19- 4.11 (m, 1H), 4.04 (ddd, 1H), 3.79 (ddd, 1H), 3.72 (s, 3H), 3.24 (dd, 1H), 3.02 (ddd, 1H), 2.95-2.85 (m, 2H), 2.00- 1.80 (m, 2H). | 1.55 min, [MH]+ = 339 | 2 | 7-(1-methyl-1H-pyrrol-3-yl)- 5-[(1,4-oxazepan-2- yl)methoxy]-1,6- naphthyridine |
| 2.49 | 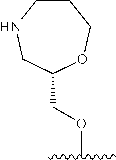 [SMILES: *OC[C@@H]1CNCCCO1] | 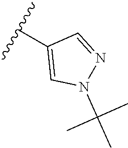 [SMILES: *c1cnn(C(C)(C)C)c1] | 1H NMR (400 MHz, Methanol-d4) δ 8.92 (dd, 1H), 8.59 (ddd, 1H), 8.36 (d, 1H), 8.11 (d, 1H), 7.61 (d, 1H), 7.49 (dd, 1H), 4.65 (dd, 1H), 4.56 (dd, 1H), 4.17 (dddd, 1H), 4.05 (ddd, 1H), 3.80 (ddd, 1H), 3.24 (dd, 1H), 3.02 (ddd, 1H), 2.97-2.88 (m, 2H), 2.02-1.83 (m, 2H), 1.65 (s, 9H). | 1.72 min, [MH]+ = 382 | 2 | 7-(1-tert-butyl-1H-pyrazol- 4-yl)-5-{[(2S)-1,4-oxazepan- 2-yl]methoxy}-1,6- naphthyridine |
| 2.50 | 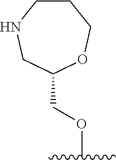 [SMILES: *OC[C@@H]1CNCCCO1] | 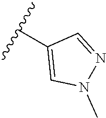 [SMILES: *c1cnn(C)c1] | 1H NMR (400 MHz, Methanol-d4) δ 8.91 (dd, 1H), 8.59 (ddd, 1H), 8.24 (s, 1H), 8.08 (d, 1H), 7.58 (d, 1H), 7.50 (dd, 1H), 4.63 (dd, 1H), 4.55 (dd, 1H), 4.16 (dddd, 1H), 4.04 (ddd, 1H), 3.96 (s, 3H), 3.79 (ddd, 1H), 3.24 (dd, 1H), 3.03 (ddd, 1H), 2.97-2.86 (m, 2H), 2.01-1.83 (m, 2H). | 1.50 min, [MH]+ = 340 | 2 | 7-(1-methyl-1H-pyrazol-4- yl)-5-{[(2S)-1,4-oxazepan-2- yl]methoxy}-1,6- naphthyridine |
| 2.51 | 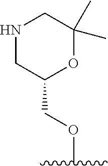 [SMILES: *OC[C@@H]1CNCC(C)(C)O1] | 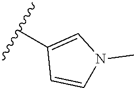 [SMILES: *c1ccn(C)c1] | 1H NMR (400 MHz, Methanol-d4) δ 8.84 (dd, 1H), 8.52 (ddd, 1H), 7.44 (d, 1H), 7.43-7.38 (m, 2H), 6.70 (app t, 1H), 6.64 (dd, 1H), 4.55 (dd, 1H), 4.47 (dd, 1H), 4.32- 4.22 (m, 1H), 3.72 (s, 3H), 3.08 (ddd, 1H), 2.71 (dd, 1H), 2.65-2.54 (m, 2H), 1.36 (s, 3H), 1.19 (s, 3H). | 1.68 min, [MH]+ = 353 | 2 | 5-{[(2S)-6,6- dimethylmorpholin-2- yl]methoxy}-7-(1-methyl- 1H-pyrrol-3-yl)-1,6- naphthyridine |
| 2.52 | 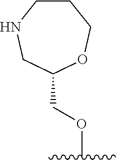 [SMILES: *OC[C@@H]1CNCCCO1] | 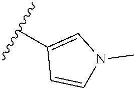 [SMILES: *c1ccn(C)c1] | 1H NMR (400 MHz, Methanol-d4) δ 8.84 (dd, 1H), 8.54 (ddd, 1H), 7.45 (d, 1H), 7.44-7.38 (m, 2H), 6.70 (app t, 1H), 6.65 (dd, 1H), 4.62 (dd, 1H), 4.54 (dd, 1H), 4.16 (dddd, 1H), 4.04 (ddd, 1H), 3.79 (ddd, 1H), 3.72 (s, 3H), 3.24 (dd, 1H), 3.02 (ddd, 1H), 2.97-2.85 (m, 2H), 2.02- 1.83 (m, 2H). | 1.56 min, [MH]+ = 339 | 2 | 7-(1-methyl-1H-pyrrol-3-yl)- 5-{[(2S)-1,4-oxazepan-2- yl]methoxy}-1,6- naphthyridine |
| 2.53 | 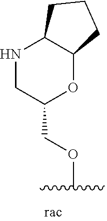 [SMILES: *OC[C@@H]1CN[C@H]2CCC[C@H]2O1] | 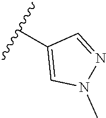 [SMILES: *c1cnn(C)c1] | 1H NMR (400 MHz, Methanol-d4) δ 8.92 (dd, 1H), 8.59 (ddd, 1H), 8.25 (s, 1H), 8.09 (d, 1H), 7.58 (d, 1H), 7.50 (dd, 1H), 4.75 (dd, 1H), 4.61 (dd, 1H), 4.30-4.18 (m, 2H), 3.96 (s, 3H), 3.16-3.06 (m, 2H), 2.74 (dd, 1H), 2.09-1.98 (m, 1H), 1.94-1.79 (m, 2H), 1.77-1.66 (m, 1H), 1.65-1.54 (m, 2H). | 1.62 min, [MH]+ = 366 | 2 | 5-{[rel-(2R,4aR,7aS)- octahydrocyclopenta[b][1,4] oxazin-2-yl]methoxy}-7-(1- methyl-1H-pyrazol-4-yl)- 1,6-naphthyridine |
| 2.54 | 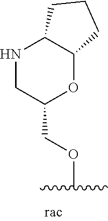 [SMILES: *OC[C@@H]1CN[C@@H]2CCC[C@@H]2O1] | 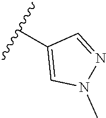 [SMILES: *c1cnn(C)c1] | 1H NMR (400 MHz, Methanol-d4) δ 8.92 (dd, 1H), 8.58 (ddd, 1H), 8.24 (s, 1H), 8.08 (d, 1H), 7.58 (d, 1H), 7.50 (dd, 1H), 4.63-4.52 (m, 2H), 4.02-3.87 (m, 5H), 3.12 (ddd, 1H), 2.96 (dd, 1H), 2.84 (dd, 1H), 2.09-1.93 (m, 1H), 1.94 1.78 (m, 2H), 1.78-1.56 (m, 3H). | 1.67 min, [MH]+ = 366 | 2 | 5-{[rel-(2S,4aR,7aS)- octahydrocyclopenta[b][1,4] oxazin-2-yl]methoxy}-7-(1- methyl-1H-pyrazol-4-yl)- 1,6-naphthyridine |
| 2.55 | 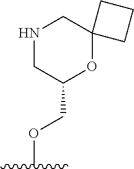 [SMILES: *OC[C@@H]1CNCC2(CCC2)O1] | 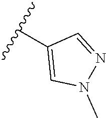 [SMILES: *c1cnn(C)c1] | 1H NMR (400 MHz, DMSO-d6) δ 9.00 (dd, 1H), 8.46 (ddd, 1H), 8.37 (d, 1H), 8.12 (d, 1H), 7.69 (d, 1H), 7.52 (dd, 1H), 4.55-4.41 (m, 2H), 3.91 (s, 3H), 3.89-3.79 (m, 1H), 2.92 (dd, 1H), 2.85 (d, 1H), 2.57-2.52 (m, 1H), 2.48- 2.43 (m, 1H), 2.27-2.14 (m, 1H), 2.01-1.84 (m, 2H), 1.84-1.65 (m, 2H), 1.63-1.47 (m, 1H). | 1.68 min, [MH]+ = 366 | 2 | 7-(1-methyl-1H-pyrazol-4- yl)-5-{[(6S)-5-oxa-8- azaspiro[3.5]nonan-6- yl]methoxy}-1,6- naphthyridine |
| 2.56 | 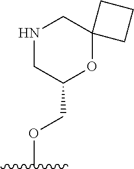 [SMILES: *OC[C@@H]1CNCC2(CCC2)O1] | 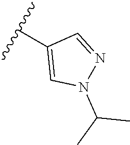 [SMILES: *c1cnn(C(C)C)c1] | 1H NMR (400 MHz, DMSO-d6) δ 8.99 (dd, 1H), 8.48- 8.41 (m, 2H), 8.12 (d, 1H), 7.70 (d, 1H), 7.51 (dd, 1H), 4.63- 4.39 (m, 3H), 3.89-3.79 (m, 1H), 2.92 (dd, 1H), 2.85 (d, 1H), 2.61-2.52 (m, 1H), 2.50-2.43 (m, 1H), 2.25-2.15 (m, 1H), 2.01-1.84 (m, 2H), 1.84-1.63 (m, 2H), 1.64- 1.49 (m, 1H), 1.47 (d, 6H). | 1.82 min, [MH]+ = 394 | 2 | 5-{[(6S)-5-oxa-8- azaspiro[3.5]nonan-6- yl]methoxy}-7-[1-(propan-2- yl)-1H-pyrazol-4-yl]-1,6- naphthyridine |
| 2.57 | 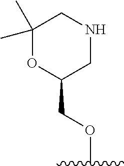 [SMILES: *OC[C@@H]1CNCC(C)(C)O1] | 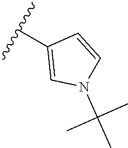 [SMILES: *c1ccn(C(C)(C)C)c1] | 1H NMR (400 MHz, Methanol-d4) δ 8.85 (dd, 1H), 8.53 (ddd, 1H), 7.64 (dd, 1H), 7.47 (d, 1H), 7.42 (dd, 1H), 6.97 (dd, 1H), 6.68 (dd, 1H), 4.59 (dd, 1H), 4.47 (dd, 1H), 4.33 4.22 (m, 1H), 3.09 (ddd, 1H), 2.71 (dd, 1H), 2.65-2.56 (m, 2H), 1.60 (s, 9H), 1.37 (s, 3H), 1.20 (s, 3H). | 1.94 min, [MH]+ = 395 | 2 | 7-(1-tert-butyl-1H-pyrrol-3- yl)-5-{[(2S)-6,6- dimethylmorpholin-2- yl]methoxy}-1,6- naphthyridine |
| 2.58 | 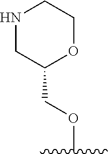 [SMILES: *OC[C@@H]1CNCCO1] | 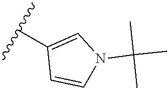 [SMILES: *c1ccn(C(C)(C)C)c1] | 1H NMR (400 MHz, Methanol-d4) δ 8.85 (dd, 1H), 8.55 (ddd, 1H), 7.63 (dd, 1H), 7.47 (d, 1H), 7.42 (dd, 1H), 6.97 (dd, 1H), 6.68 (dd, 1H), 4.61 (d, 1H), 4.60-4.59 (m, 1H), 4.06-3.99 (m, 1H), 3.97-3.89 (m, 1H), 3.69 (ddd, 1H), 3.09 (dd, 1H), 2.92-2.76 (m, 3H), 1.60 (s, 9H). | 1.80 min, [MH]+ = 367 | 2 | 7-(1-tert-butyl-1H-pyrrol-3- yl)-5-{[(2S)-morpholin-2- yl]methoxy}-1,6- naphthyridine |
| 2.59 | 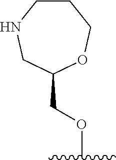 [SMILES: *OC[C@H]1CNCCCO1] | 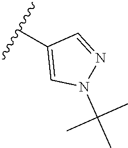 [SMILES: *c1cnn(C(C)(C)C)c1] | H NMR (400 MHz, Methanol-d4) δ 8.91 (dd, 1H), 8.58 (dddd, 1H), 8.35 (d, 1H), 8.11 (d, 1H), 7.60 (d, 1H), 7.48 (dd, 1H), 4.64 (dd, 1H), 4.55 (dd, 1H), 4.16 (dddd, 1H), 4.05 (ddd, 1H), 3.80 (ddd, 1H), 3.24 (dd, 1H), 3.02 (ddd, 1H), 2.97-2.88 (m, 2H), 2.01-1.83 (m, 2H), 1.65 (s, 9H). | 1.78 min, [MH]+ = 382 | 2 | 7-(1-tert-butyl-1H-pyrazol- 4-yl)-5-{[(2R)-1,4- oxazepan-2-yl]methoxy}- 1,6-naphthyridine |
| 2.60 | 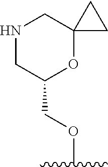 [SMILES: *OC[C@@H]1CNCC2(CC2)O1] | 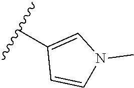 [SMILES: *c1ccn(C)c1] | 1H NMR (400 MHz, Methanol-d4) δ 8.81 (dd, 1H), 8.44 (ddd, 1H), 7.40 (d, 1H), 7.39-7.29 (m, 2H), 6.69 (app t, 1H), 6.62 (dd, 1H), 4.54 (dd, 1H), 4.48 (dd, 1H), 4.18- 4.08 (m, 1H), 3.70 (s, 3H), 3.27 (dd, 1H), 3.11 (ddd, 1H), 2.81 (dd, 1H), 2.31 (dd, 1H), 0.93-0.81 (m, 1H), 0.79- 0.68 (m, 1H), 0.65-0.54 (m, 2H). | 1.73 min, [MH]+ = 351 | 2 | 7-(1-methyl-1H-pyrrol-3-yl)- 5-{[(5S)-4-oxa-7- azaspiro[2.5]octan-5- yl]methoxy}-1,6- naphthyridine |
| 2.61 | 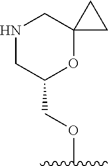 [SMILES: *OC[C@@H]1CNCC2(CC2)O1] | 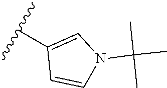 [SMILES: *c1ccn(C(C)(C)C)c1] | 1H NMR (400 MHz, Methanol-d4) δ 8.85 (dd, 1H), 8.52 (ddd, 1H), 7.62 (dd, 1H), 7.47 (d, 1H), 7.41 (dd, 1H), 6.97 (dd, 1H), 6.68 (dd, 1H), 4.61 (dd, 1H), 4.54 (dd, 1H), 4.23- 4.13 (m, 1H), 3.34-3.25 (m, 1H), 3.15 (ddd, 1H), 2.85 (dd, 1H), 2.32 (dd, 1H), 1.60 (s, 9H), 0.92-0.81 (m, 1H), 0.79- 0.72 (m, 1H), 0.67-0.56 (m, 2H). | 1.96 min, [MH]+ = 393 | 2 | 7-(1-tert-butyl-1H-pyrrol-3- yl)-5-{[(5S)-4-oxa-7- azaspiro[2.5]octan-5- yl]methoxy}-1,6- naphthyridine |
| 2.62 | 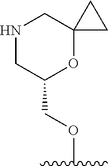 [SMILES: *OC[C@@H]1CNCC2(CC2)O1] | 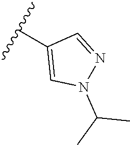 [SMILES: *c1cnn(C(C)C)c1] | 1H NMR (400 MHz, Methanol-d4) mixture of rotamers & 8.96-8.80 (m, 1H), 8.63-8.44 (m, 1H), 8.34-8.21 (m, 1H), 8.16-8.02 (m, 1H), 7.61-7.53 (m, 1H), 7.53-7.35 (m, 1H), 4.76-4.03 (m, 4H), 3.69-3.17 (m, 2H), 3.15- 2.65 (m, 1H), 2.84-2.27 (m, 1H), 1.59-1.50 (m, 6H), 1.08-0.55 (m, 4H). | 1.73 min, [MH]+ = 380 | 2 | 5-{[(5S)-4-oxa-7- azaspiro[2.5]octan-5- yl]methoxy}-7-[1-(propan-2- yl)-1H-pyrazol-4-yl]-1,6- naphthyridine |
| 2.63 | 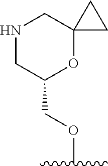 [SMILES: *OC[C@@H]1CNCC2(CC2)O1] | 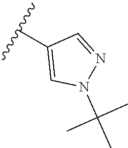 [SMILES: *c1cnn(C(C)(C)C)c1] | 1H NMR (400 MHz, Methanol-d4) δ 8.88 (dd, 1H), 8.51 (ddd, 1H), 8.32 (d, 1H), 8.08 (d, 1H), 7.55 (d, 1H), 7.45 (dd, 1H), 4.58 (dd, 1H), 4.52 (dd, 1H), 4.21-4.11 (m, 1H), 3.31-3.26 (m, 1H), 3.17-3.10 (m, 1H), 2.85 (dd, 1H), 2.32 (d, 1H), 1.64 (s, 9H), 0.97-0.81 (m, 1H), 0.78-0.69 (m, 1H), 0.66-0.55 (m, 2H) | 1.81 min, [MH]+ = 394 | 2 | 7-(1-tert-butyl-1H-pyrazol- 4-yl)-5-{[(5S)-4-oxa-7- azaspiro[2.5]octan-5- yl]methoxy}-1,6- naphthyridine |
| 2.64 | 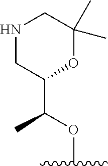 [SMILES: *O[C@@H](C)[C@@H]1CNCC(C)(C)O1] | 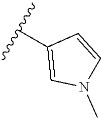 [SMILES: *c1ccn(C)c1] | 1H NMR (400 MHz, Methanol-d4) δ 8.83 (dd, 1H), 8.52 (ddd, 1H), 7.48-7.33 (m, 3H), 6.74-6.66 (m, 1H), 6.64 (dd, 1H), 5.56 (qd, 1H), 4.09 (ddd, 1H), 3.72 (s, 3H), 2.94 (ddd, 1H), 2.70-2.50 (m, 3H), 1.44 (d, 3H), 1.34 (s, 3H), 1.17 (s, 3H). | 1.77 min, [MH]+ = 367 | 2 | 5-[(1S)-1-[(2S)-6,6- dimethylmorpholin-2- yl]ethoxy]-7-(1-methyl-1H- pyrrol-3-yl)-1,6- naphthyridine |
| 2.65 | 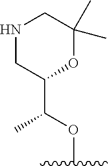 [SMILES: *O[C@H](C)[C@@H]1CNCC(C)(C)O1] | 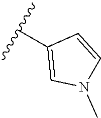 [SMILES: *c1ccn(C)c1] | 1H NMR (400 MHz, Methanol-d4) δ 8.83 (dd, 1H), 8.50 (ddd, 1H), 7.48-7.35 (m, 3H), 6.73-6.68 (m, 1H), 6.64 (dd, 1H), 5.45 (p, 1H), 4.01-3.88 (m, 1H), 3.72 (s, 3H), 3.08 (ddd, 1H), 2.72-2.65 (m, 1H), 2.60-2.50 (m, 2H), 1.46 (d, 3H), 1.33 (s, 3H), 1.16 (s, 3H). | 1.77 min, [MH]+ = 367 | 2 | 5-[(1R)-1-[(2S)-6,6- dimethylmorpholin-2- yl]ethoxy]-7-(1-methyl-1H- pyrrol-3-yl)-1,6- naphthyridine |
| 2.66 | 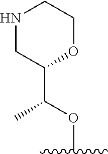 [SMILES: *O[C@H](C)[C@@H]1CNCCO1] | 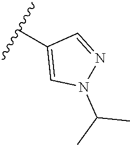 [SMILES: *c1cnn(C(C)C)c1] | 1H NMR (400 MHz, Methanol-d4) d 8.93 (dd, 1H), 8.58 (ddd, 1H), 8.30 (d, 1H), 8.10 (d, 1H), 7.60 (d, 1H), 7.50 (dd, 1H), 5.64-5.56 (m, 1H), 4.62 (hept, 1H), 3.97-3.89 (m, 1H), 3.79 (ddd, 1H), 3.67 (ddd, 1H), 3.13 (dd, 1H), 2.91 2.74 (m, 3H), 1.56 (d, 6H), 1.50 (d, 3H). | 1.69 min, [MH]+ = 368 | 2 | 5-[(1R)-1-[(2S)-morpholin- 2-yl]ethoxy]-7-[1-(propan-2- yl)-1H-pyrazol-4-yl]-1,6- naphthyridine |
| 2.67 | 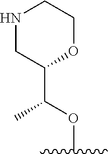 [SMILES: *O[C@H](C)[C@@H]1CNCCO1] | 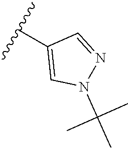 [SMILES: *c1cnn(C(C)(C)C)c1] | 1H NMR (400 MHz, Methanol-d4) δ 8.91 (ddd, 1H), 8.56 (dddd, 1H), 8.34 (d, 1H), 8.10 (d, 1H), 7.59 (d, 1H), 7.48 (ddd, 1H), 5.60-5.53 (m, 1H), 3.95-3.87 (m, 1H), 3.77 (ddd, 1H), 3.65 (ddd, 1H), 3.10 (dd, 1H), 2.88-2.71 (m, 3H), 1.65 (s, 9H), 1.49 (d, 3H). | 1.79 min, [MH]+ = 382 | 2 | 7-(1-tert-butyl-1H-pyrazol- 4-yl)-5-[(1R)-1-[(2S)- morpholin-2-yl]ethoxy]-1,6- naphthyridine |
| 2.68 | 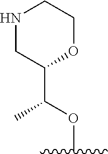 [SMILES: *O[C@H](C)[C@@H]1CNCCO1] | 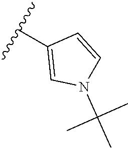 [SMILES: *c1ccn(C(C)(C)C)c1] | 1H NMR (400 MHz, Methanol-d4) δ 8.85 (ddd, 1H), 8.52 (ddd, 1H), 7.61 (dd, 1H), 7.46 (d, 1H), 7.41 (ddd, 1H), 6.97 (app td, 1H), 6.68 (dd, 1H), 5.56 (dq, 1H), 3.96-3.89 (m, 1H), 3.77 (ddd, 1H), 3.71-3.61 (m, 1H), 3.10 (dd, 1H), 2.89-2.71 (m, 3H), 1.60 (s, 9H), 1.50 (d, 3H). | 1.86 min, [MH]+ = 381 | 2 | 7-(1-tert-butyl-1H-pyrrol-3- yl)-5-[(1R)-1-[(2S)- morpholin-2-yl]ethoxy]-1,6- naphthyridine |
| 2.69 | 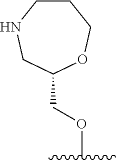 [SMILES: *OC[C@@H]1CNCCCO1] | 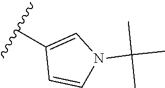 [SMILES: *c1ccn(C(C)(C)C)c1] | 1H NMR (400 MHz, Methanol-d4) δ 8.85 (dd, 1H), 8.54 (ddd, 1H), 7.62 (dd, 1H), 7.47 (d, 1H), 7.41 (dd, 1H), 6.96 (dd, 1H), 6.67 (dd, 1H), 4.63 (dd, 1H), 4.54 (dd, 1H), 4.21- 4.13 (m, 1H), 4.05 (ddd, 1H), 3.80 (ddd, 1H), 3.24 (dd, 1H), 3.02 (ddd, 1H), 2.97-2.86 (m, 2H), 2.02-1.81 (m, 2H), 1.59 (s, 9H). | 1.79 min, [MH]+ = 381 | 2 | 7-(1-tert-butyl-1H-pyrrol-3- yl)-5-{[(2S)-1,4-oxazepan-2- yl]methoxy}-1,6- naphthyridine |
| 2.70 | 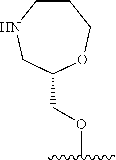 [SMILES: *OC[C@@H]1CNCCCO1] | 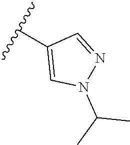 [SMILES: *c1cnn(C(C)C)c1] | 1H NMR (400 MHz, Methanol-d4) δ 8.91 (dd, 1H), 8.59 (ddd, 1H), 8.31 (d, 1H), 8.10 (d, 1H), 7.59 (d, 1H), 7.49 (dd, 1H), 4.67-4.51 (m, 3H), 4.16 (dddd, 1H), 4.05 (ddd, 1H), 3.80 (ddd, 1H), 3.24 (dd, 1H), 3.02 (ddd, 1H), 2.96- 2.86 (m, 2H), 2.00-1.83 (m, 2H), 1.55 (d, 6H). | 1.65 min, [MH]+ = 368 | 2 | 5-{[(2S)-1,4-oxazepan-2- yl]methoxy}-7-[1-(propan-2- yl)-1H-pyrazol-4-yl]-1,6- naphthyridine |
| 2.71 | 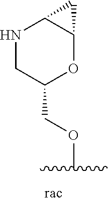 [SMILES: *OC[C@@H]1CN[C@@H]2C[C@@H]2O1] | 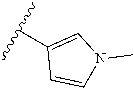 [SMILES: *c1ccn(C)c1] | 1H NMR (400 MHz, Methanol-d4) δ 8.82 (dd, 1H), 8.52 (ddd, 1H), 7.43 (d, 1H), 7.41-7.36 (m, 2H), 6.69 (app t, 1H), 6.64 (dd, 1H), 4.78 (dd, 1H), 4.56 (dd, 1H), 4.09- 3.99 (m, 1H), 3.71 (s, 3H), 3.49 (td, 1H), 2.95 (dd, 1H), 2.72 (dd, 1H), 2.37 (ddd, 1H), 0.85-0.76 (m, 1H), 0.85- 0.76 (m, 1H). | 1.55 min, [MH]+ = 337 | 2 | 7-(1-methyl-1H-pyrrol-3-yl)- 5-{[(rel-1S,3S,6R)-2-oxa-5- azabicyclo[4.1.0]heptan-3- yl]methoxy}-1,6- naphthyridine |
| 2.72 | 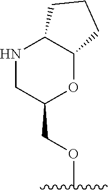 [SMILES: *OC[C@H]1CN[C@@H]2CCC[C@@H]2O1] | 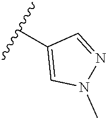 [SMILES: *c1cnn(C)c1] | 1H NMR (400 MHz, Methanol-d4) δ 8.92 (dd, 1H), 8.63- 8.58 (m, 1H), 8.26 (s, 1H), 8.10 (d, 1H), 7.60 (d, 1H), 7.51 (dd, 1H), 4.75 (dd, 1H), 4.62 (dd, 1H), 4.30-4.18 (m, 2H), 3.96 (s, 3H), 3.15-3.09 (m, 2H), 2.74 (dd, 1H), 2.11-1.97 (m, 1H), 1.96-1.82 (m, 2H), 1.76-1.67 (m, 1H), 1.65- 1.53 (m, 2H), | 1.58 min, [MH]+ = 366 | 2 | 5-{[(2R,4aR,7aS)- octahydrocyclopenta[b][1,4] oxazin-2-yl]methoxy}-7-(1- methyl-1H-pyrazol-4-yl)- 1,6-naphthyridine |
| 2.73 | 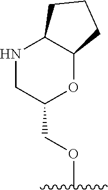 [SMILES: *OC[C@@H]1CN[C@H]2CCC[C@H]2O1] | 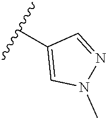 [SMILES: *c1cnn(C)c1] | 1H NMR (400 MHz, Methanol-d4) δ 8.92 (dd, 1H), 8.61 (ddd, 1H), 8.26 (d, 1H), 8.09 (d, 1H), 7.60 (d, 1H), 7.51 (dd, 1H), 4.75 (dd, 1H), 4.61 (dd, 1H), 4.31-4.20 (m, 2H), 3.96 (s, 3H), 3.15-3.06 (m, 2H), 2.74 (dd, 1H), 2.10-1.96 (m, 1H), 1.92-1.81 (m, 2H), 1.78-1.67 (m, 1H), 1.66- 1.51 (m, 2H). | 1.58 min, [MH]+ = 366 | 2 | 5-{[(2S,4aS,7aR)- octahydrocyclopenta[b][1,4] oxazin-2-yl]methoxy}-7-(1- methyl-1H-pyrazol-4-yl)- 1,6-naphthyridine |
| 2.74 | 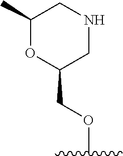 [SMILES: *OC[C@@H]1CNC[C@H](C)O1] | 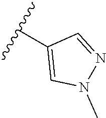 [SMILES: *c1cnn(C)c1] | 1H NMR (400 MHz, Methanol-d4) δ 8.86 (dd, 1H), 8.48 (ddd, 1H), 8.14 (s, 1H), 8.01 (d, 1H), 7.47 (d, 1H), 7.43 (dd, 1H), 4.59-4.46 (m, 2H), 4.08-3.98 (m, 1H), 3.93 (s, 3H), 3.76-3.63 (m, 1H), 3.05 (ddd, 1H), 2.86 (ddd, 1H), 2.66 (dd, 1H), 2.43 (dd, 1H), 1.16 (d, 3H). | 1.53 min, [MH]+ = 340 | 2 | 7-(1-methyl-1H-pyrazol-4- yl)-5-{[(25,6S)-6- methylmorpholin-2- yl]methoxy}-1,6- naphthyridine |
| 2.75 | 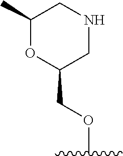 [SMILES: *OC[C@@H]1CNC[C@H](C)O1] | 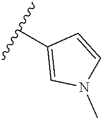 [SMILES: *c1ccn(C)c1] | 1H NMR (400 MHz, Methanol-d4) δ 8.83 (dd, 1H), 8.51 (ddd, 1H), 7.43 (d, 1H), 7.43-7.36 (m, 3H), 6.69 (app t, 1H), 6.64 (dd, 1H), 4.63-4.50 (m, 2H), 4.09-3.99 (m, 1H), 3.75-3.63 (m, 4H), 3.05 (ddd, 1H), 2.85 (ddd, 1H), 2.66 (dd, 1H), 2.42 (dd, 1H), 1.15 (d, 3H). | 1.59 min, [MH]+ = 339 | 2 | 7-(1-methyl-1H-pyrrol-3-yl)- 5-{[(2,6S)-6- methylmorpholin-2- yl]methoxy}-1,6- naphthyridine |
| 2.76 | 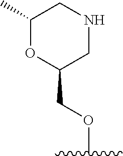 [SMILES: *OC[C@@H]1CNC[C@@H](C)O1] | 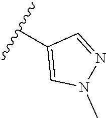 [SMILES: *c1cnn(C)c1] | 1H NMR (400 MHz, Methanol-d4) δ 8.92 (dd, 1H), 8.61 (ddd, 1H), 8.28 (s, 1H), 8.11 (d, 1H), 7.59 (d, 1H), 7.50 (dd, 1H), 5.06 (dd, 1H), 4.66 (dd, 1H), 4.32-4.23 (m, 1H), 4.11-4.02 (m, 1H), 3.96 (s, 3H), 3.05 (dd, 1H), 2.98-2.89 (m, 2H), 2.54 (dd, 1H), 1.14 (d, 3H). | 1.51 min, [MH]+ = 340 | 2 | 7-(1-methyl-1H-pyrazol-4- yl)-5-{[(2S,6R)-6- methylmorpholin-2- yl]methoxy}-1,6- naphthyridine |
| 2.77 | 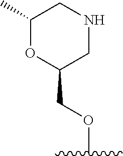 [SMILES: *OC[C@@H]1CNC[C@@H](C)O1] | 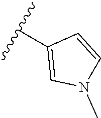 [SMILES: *c1ccn(C)c1] | 1H NMR (400 MHz, Methanol-d4) δ 8.84 (dd, 1H), 8.55 (ddd, 1H), 7.48-7.43 (m, 2H), 7.41 (dd, 1H), 6.71 (app t, 1H), 6.66 (dd, 1H), 5.01 (dd, 1H), 4.67 (dd, 1H), 4.33- 4.23 (m, 1H), 4.13-4.02 (m, 1H), 3.72 (s, 3H), 3.05 (dd, 1H), 2.99-2.89 (m, 2H), 2.55 (dd, 1H), 1.16 (d, 3H). | 1.56 min, [MH]+ = 339 | 2 | 7-(1-methyl-1H-pyrrol-3-yl)- 5-{[(2S,6R)-6- methylmorpholin-2- yl]methoxy}-1,6- naphthyridine |
| 2.78 | 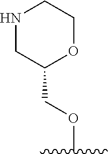 [SMILES: *OC[C@@H]1CNCCO1] | 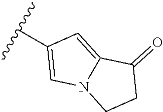 [SMILES: *c1cc2n(c1)CCC2=O] | 1H NMR (600 MHz, Methanol-d4) δ 8.93 (dd, 1H), 8.61 (d, 1H), 7.99 (s, 1H), 7.66 (s, 1H), 7.52 (dd, 1H), 7.30 (s, 1H), 4.63 (d, 2H), 4.45 (t, 2H), 4.07-4.00 (m, 1H), 3.93 (dd, 1H), 3.70 (td, 1H), 3.14 (t, 2H), 3.11-3.07 (m, 1H), 2.91- 2.76 (m, 3H) | 1.53 min, [MH]+ = 365 | 2 | 6-(5-{[(2S)-morpholin-2- yl]methoxy}-1,6- naphthyridin-7-yl)-2,3- dihydro-1H-pyrrolizin-1-one |
| 2.79 | 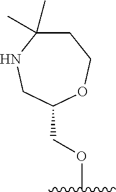 [SMILES: *OC[C@@H]1CNC(C)(C)CCO1] | 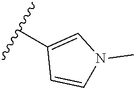 [SMILES: *c1ccn(C)c1] | 1H NMR (400 MHz, Methanol-d4) δ 8.84 (dd, 1H), 8.53 (ddd, 1H), 7.45 (d, 1H), 7.43-7.38 (m, 2H), 6.71 (app t, 1H), 6.65 (dd, 1H), 4.61 (dd, 1H), 4.51 (dd, 1H), 4.18- 4.08 (m, 1H), 3.93 (ddd, 1H), 3.81 (ddd, 1H), 3.72 (s, 3H), 3.07 (dd, 1H), 2.99 (dd, 1H), 1.96-1.79 (m, 2H), 1.22 (s, 3H), 1.16 (s, 3H). | 1.62 min, [MH]+ = 367 | 2 | 5-{[(2S)-5,5-dimethyl-1,4- oxazepan-2-yl]methoxy}-7- (1-methyl-1H-pyrrol-3-yl)- 1,6-naphthyridine |
| 2.80 | 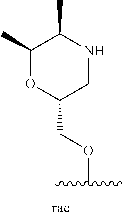 [SMILES: *OC[C@H]1CN[C@H](C)[C@H](C)O1] | 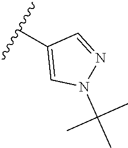 [SMILES: *c1cnn(C(C)(C)C)c1] | 1H NMR (400 MHz, Methanol-d4) δ 8.92 (dd, 1H), 8.59 (ddd, 1H), 8.36 (d, 1H), 8.11 (d, 1H), 7.61 (d, 1H), 7.50 (dd, 1H), 4.63 (dd, 1H), 4.54 (dd, 1H), 4.29-4.16 (m, 1H), 4.00-3.88 (m, 1H), 3.14-3.05 (m, 2H), 2.80 (dd, 1H), 1.65 (s, 9H), 1.27 (d, 3H), 0.98 (d, 3H). | 1.78 min, [MH]+ = 396 | 2 | 7-(1-tert-butyl-1H-pyrazol- 4-yl)-5-{[rel-(2S,5S,6R)-5,6- dimethylmorpholin-2- yl]methoxy}-1,6- naphthyridine |
| 2.81 | 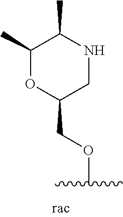 [SMILES: *OC[C@@H]1CN[C@H](C)[C@H](C)O1] | 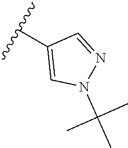 [SMILES: *c1cnn(C(C)(C)C)c1] | 1H NMR (400 MHz, Methanol-d4) δ 8.92 (dd, 1H), 8.58 (ddd, 1H), 8.36 (d, 1H), 8.11 (d, 1H), 7.61 (d, 1H), 7.50 (dd, 1H), 4.67-4.55 (m, 2H), 4.13-4.02 (m, 1H), 3.94- 3.84 (m, 1H), 3.02 (dd, 1H), 2.89-2.74 (m, 2H), 1.65 (s, 9H), 1.21 (d, 3H), 1.11 (d, 3H). | 1.82 min, [MH]+ = 396 | 2 | 7-(1-tert-butyl-1H-pyrazol- 4-yl)-5-{[rel-(2S,5R,6S)-5,6- dimethylmorpholin-2- yl]methoxy}-1,6- naphthyridine |
| 2.82 | 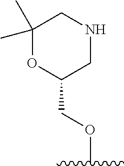 [SMILES: *OC[C@H]1CNCC(C)(C)O1] | 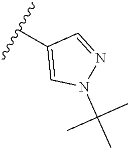 [SMILES: *c1cnn(C(C)(C)C)c1] | 1H NMR (400 MHz, Methanol-d4) δ 8.91 (dd, 1H), 8.57 (ddd, 1H), 8.36 (d, 1H), 8.11 (d, 1H), 7.60 (d, 1H), 7.49 (dd, 1H), 4.59 (dd, 1H), 4.49 (dd, 1H), 4.32-4.20 (m, 1H), 3.08 (ddd, 1H), 2.74-2.57 (m, 3H), 1.65 (s, 9H), 1.36 (s, 3H), 1.19 (s, 3H). | 1.82 min, [MH]+ = 396 | 2 | 7-(1-tert-butyl-1H-pyrazol- 4-yl)-5-{[(2R)-6,6- dimethylmorpholin-2- yl]methoxy}-1,6- naphthyridine |
| 2.83 | 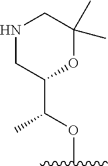 [SMILES: *O[C@H](C)[C@@H]1CNCC(C)(C)O1] | 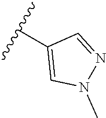 [SMILES: *c1cnn(C)c1] | 1H NMR (400 MHz, Methanol-d4) δ 8.91 (dd, 1H), 8.56 (ddd, 1H), 8.23 (s, 1H), 8.07 (d, 1H), 7.57 (d, 1H), 7.49 (dd, 1H), 5.48 (p, 1H), 4.01-3.92 (m, 4H), 3.08 (ddd, 1H), 2.72 2.52 (m, 3H), 1.47 (d, 3H), 1.33 (s, 3H), 1.16 (s, 3H). | 1.71 min, [MH]+ = 368 | 2 | 5-[(1R)-1-[(2S)-6,6- dimethylmorpholin-2- yl]ethoxy]-7-(1-methyl-1H- pyrazol-4-yl)-1,6- naphthyridine |
| 2.84 | 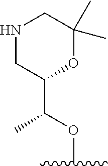 [SMILES: *O[C@H](C)[C@@H]1CNCC(C)(C)O1] | 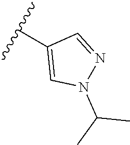 [SMILES: *c1cnn(C(C)C)c1] | 1H NMR (400 MHz, Methanol-d4) δ 8.91 (dd, 1H), 8.60- 8.54 (m, 1H), 8.29 (d, 1H), 8.09 (d, 1H), 7.58 (d, 1H), 7.49 (dd, 1H), 5.49 (p, 1H), 4.61 (hept, 1H), 4.01-3.92 (m, 1H), 3.09 (ddd, 1H), 2.69 (dd, 1H), 2.62-2.53 (m, 2H), 1.55 (d, 6H), 1.47 (d, 3H), 1.33 (s, 3H), 1.16 (s, 3H). | 1.85 min, [MH]+ = 396 | 2 | 5-[(1R)-1-[(2S)-6,6- dimethylmorpholin-2- yl]ethoxy]-7-[1-(propan-2- yl)-1H-pyrazol-4-yl]-1,6- naphthyridine |
| 2.85 | 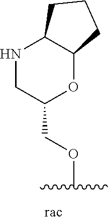 [SMILES: *OC[C@@H]1CN[C@H]2CCC[C@H]2O1] | 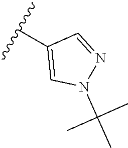 [SMILES: *c1cnn(C(C)(C)C)c1] | 1H NMR (400 MHz, Methanol-d4) δ 8.91 (dd, 1H), 8.59 (ddd, 1H), 8.36 (d, 1H), 8.12 (d, 1H), 7.60 (d, 1H), 7.49 (dd, 1H), 4.76 (dd, 1H), 4.60 (dd, 1H), 4.30-4.18 (m, 2H), 3.15-3.05 (m, 2H), 2.75 (dd, 1H), 2.12-1.99 (m, 1H), 1.94-1.80 (m, 2H), 1.79-1.68 (m, 1H), 1.65 (s, 9H), 1.63- 1.52 (m, 2H). | 1.82 min, [MH]+ = 408 | 2 | 5-{[rel-(2S,4aS,7aR)- octahydrocyclopenta[b][1,4] oxazin-2-yl]methoxy}-7-(1- tert-butyl-1H-pyrazol-4-yl)- 1,6-naphthyridine |
| 2.86 | 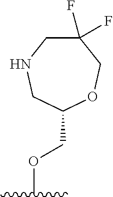 [SMILES: *OC[C@@H]1CNCC(F)(F)CO1] | 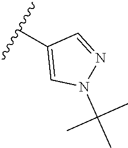 [SMILES: *c1cnn(C(C)(C)C)c1] | 1H NMR (400 MHz, Methanol-d4) δ 8.92 (dd, 1H), 8.59 (ddd, 1H), 8.36 (d, 1H), 8.12 (d, 1H), 7.62 (d, 1H), 7.51 (dd, 1H), 4.67-4.55 (m, 2H), 4.22-4.10 (m, 2H), 4.02 (ddd, 1H), 3.39-3.31 (m, 2H), 3.25-3.09 (m, 1H), 2.90 (dd, 1H), 1.65 (s, 9H). | 2.02 min, [MH]+ = 418 | 2 | 7-(1-tert-butyl-1H-pyrazol- 4-yl)-5-{[(2S)-6,6-difluoro- 1,4-oxazepan-2- yl]methoxy}-1,6- naphthyridine |
| 2.87 | 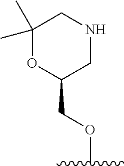 [SMILES: *OC[C@@H]1CNCC(C)(C)O1] | 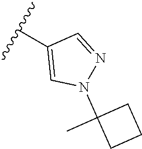 [SMILES: *c1cnn(C2(C)CCC2)c1] | 1H NMR (400 MHz, Methanol-d4) δ 8.93 (dd, 1H), 8.58 (ddd, 1H), 8.31 (d, 1H), 8.14 (d, 1H), 7.62 (d, 1H), 7.51 (dd, 1H), 4.60 (dd, 1H), 4.50 (dd, 1H), 4.31-4.24 (m, 1H), 3.09 (ddd, 1H), 2.84-2.69 (m, 3H), 2.67-2.57 (m, 2H), 2.35-2.25 (m, 2H), 2.13-1.89 (m, 2H), 1.75 (s, 3H), 1.37 (s, 3H), 1.20 (s, 3H). | 1.89 min, [MH]+ = 408 | 2 | 5-{[(2S)-6,6- dimethylmorpholin-2- yl]methoxy}-7-[1-(1- methylcyclobutyl)-1H- pyrazol-4-yl]-1,6- naphthyridine |
| 2.88 | 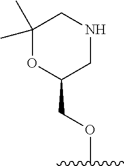 [SMILES: *OC[C@@H]1CNCC(C)(C)O1] | 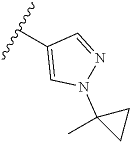 [SMILES: *c1cnn(C2(C)CC2)c1] | 1H NMR (400 MHz, Chloroform-d) δ 8.94 (dd, 1H), 8.45 (ddd, 1H), 8.06 (d, 1H), 8.01 (d, 1H), 7.58 (d, 1H), 7.34 (dd, 1H), 4.60 (dd, 1H), 4.43 (dd, 1H), 4.32-4.14 (m, 1H), 3.38-2.97 (m, 1H), 2.94-2.43 (m, 3H), 1.68 (s, 3H), 1.38 (s, 3H), 1.38-1.30 (m, 2H), 1.24 (s, 3H), 1.01-0.91 (m, 2H). | 1.77 min, [MH]+ = 394 | 2 | 5-{[(2S)-6,6- dimethylmorpholin-2- yl]methoxy}-7-[1-(1- methylcyclopropyl)-1H- pyrazol-4-yl]-1,6- naphthyridine |
| 2.89 | 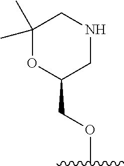 [SMILES: *OC[C@@H]1CNCC(C)(C)O1] | 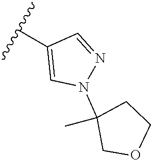 [SMILES: *c1cnn(C2(C)CCOC2)c1] | 1H NMR (400 MHz, Methanol-d4) δ 8.95-8.88 (m, 1H), 8.60-8.53 (m, 1H), 8.42 (app t, 1H), 8.16-8.11 (m, 1H), 7.60 (dd, 1H), 7.50 (dd, 1H), 4.63-4.55 (m, 1H), 4.54- 4.43 (m, 1H), 4.38 (d, 1H), 4.33-4.23 (m, 1H), 4.11-3.98 (m, 2H), 3.87 (d, 1H), 3.11 (ddd, 1H), 2.85-2.71 (m, 2H), 2.71-2.60 (m, 2H), 2.31 (ddd, 1H), 1.75 (s, 3H), 1.38 (s, 3H), 1.21 (s, 3H). | 1.73 min, [MH]+ = 424 | 2 | 7-{1-[(3,3- difluorocyclobutyl)methyl]- 1H-pyrazol-4-yl}-5-{[(2S)- morpholin-2-yl]methoxy}- 1,6-naphthyridine |
| 2.90 | 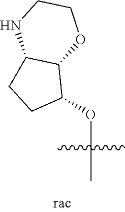 [SMILES: *O[C@@H]1CC[C@@H]2NCCO[C@@H]21] | 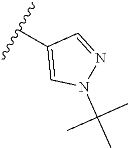 [SMILES: *c1cnn(C(C)(C)C)c1] | 1H NMR (400 MHz, Methanol-d4) δ 8.91 (dd, 1H), 8.59 (ddd, 1H), 8.36 (d, 1H), 8.11 (d, 1H), 7.59 (d, 1H), 7.49 (dd, 1H), 5.58-5.49 (m, 1H), 3.98 (ddd, 1H), 3.79-3.68 (m, 2H), 3.00-2.89 (m, 2H), 2.89-2.78 (m, 1H), 2.73- 2.60 (m, 1H), 2.02-1.89 (m, 1H), 1.80-1.68 (m, 2H), 1.67 (s, 9H). | 1.77 min, [MH]+ = 394 | 2 | 5-[rel-(4aS,7R,7aS)- octahydrocyclopenta[b][1,4] oxazin-7- yloxy]-7-(1-tert-butyl-1H- pyrazol-4-yl)-1,6- naphthyridine |
| 2.91 | 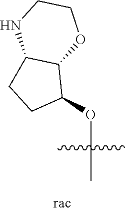 [SMILES: *O[C@H]1CC[C@@H]2NCCO[C@@H]21] | 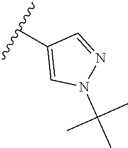 [SMILES: *c1cnn(C(C)(C)C)c1] | 1H NMR (600 MHz, Methanol-d4) δ 8.92 (dd, 1H), 8.55 (ddd, 1H), 8.41 (d, 1H), 8.16 (d, 1H), 7.62 (d, 1H), 7.50 (dd, 1H), 5.64 (ddd, 1H), 4.13 (dd, 1H), 3.96-3.90 (m, 1H), 3.62 (ddd, 1H), 3.51-3.45 (m, 1H), 3.13 (ddd, 1H), 2.75-2.69 (m, 1H), 2.65-2.57 (m, 1H), 2.17-2.10 (m, 1H), 2.03-1.96 (m, 1H), 1.96-1.89 (m, 1H), 1.66 (s, 9H). | 1.78 mins, [MH]+ = 394 | 2 | 5-[rel-(4aS,7S,7aS)- octahydrocyclopenta[b][1,4] oxazin-7- yloxy]-7-(1-tert-butyl-1H- pyrazol-4-yl)-1,6- naphthyridine |
| 2.92 | 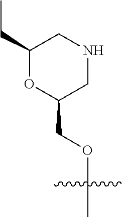 [SMILES: *OC[C@@H]1CNC[C@H](CC)O1] | 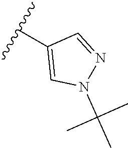 [SMILES: *c1cnn(C(C)(C)C)c1] | 1H NMR (400 MHz, Methanol-d4) δ 8.93 (dd, 1H), 8.60 (ddd, 1H), 8.37 (d, 1H), 8.13 (d, 1H), 7.63 (d, 1H), 7.51 (dd, 1H), 4.68-4.58 (m, 2H), 4.10-4.01 (m, 1H), 3.53- 3.44 (m, 1H), 3.08 (ddd, 1H), 2.93-2.85 (m, 1H), 2.70 (dd, 1H), 2.45 (dd, 1H), 1.66 (s, 9H), 1.59-1.42 (m, 2H), 0.97 (t, 3H) | 1.88 min, [MH]+ = 396. | 2 | 7-(1-tert-butyl-1H-pyrazol- 4-yl)-5-{[(2S,6S)-6- ethylmorpholin-2- yl]methoxy}-1,6- naphthyridine |
| 2.93 | 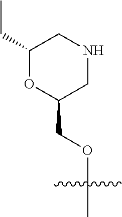 [SMILES: *OC[C@@H]1CNC[C@@H](CC)O1] | 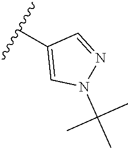 [SMILES: *c1cnn(C(C)(C)C)c1] | 1H NMR (400 MHz, Methanol-d4) δ 8.92 (dd, 1H), 8.61 (ddd, 1H), 8.43-8.39 (m, 1H), 8.15 (s, 1H), 7.62 (d, 1H), 7.50 (dd, 1H), 5.13 (dd, 1H), 4.61 (dd, 1H), 4.30-4.23 (m, 1H), 3.84-3.74 (m, 1H), 3.06 (dd, 1H), 3.00-2.89 (m, 2H), 2.60 (dd, 1H), 1.73-1.58 (m, 10H), 1.49-1.37 (m, 1H), 0.84 (t, 3H). | 1.83 min, [MH]+ = 396 | 2 | 7-(1-tert-butyl-1H-pyrazol- 4-yl)-5-{[(2S,6R)-6- ethylmorpholin-2- yl]methoxy}-1,6- naphthyridine |
| 2.94 | 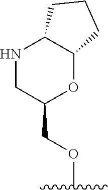 [SMILES: *OC[C@H]1CN[C@@H]2CCC[C@@H]2O1] | 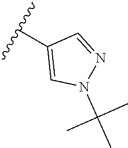 [SMILES: *c1cnn(C(C)(C)C)c1] | 1H NMR (400 MHz, Methanol-d4) δ 8.93 (dd, 1H), 8.61 (ddd, 1H), 8.38 (d, 1H), 8.14 (d, 1H), 7.63 (d, 1H), 7.51 (dd, 1H), 4.78 (dd, 1H), 4.63 (dd, 1H), 4.34-4.20 (m, 2H), 3.18-3.08 (m, 2H), 2.78 (dd, 1H), 2.11-1.99 (m, 1H), 1.96-1.81 (m, 2H), 1.78-1.69 (m, 1H), 1.66 (s, 9H), 1.64- 1.54 (m, 2H). | 1.78 min, [MH]+ = 408 | 2 | 5-{[(2S,4aS,7aR)- octahydrocyclopenta[b][1,4] oxazin-2-yl]methoxy}-7-(1- tert-butyl-1H-pyrazol-4-yl)- 1,6-naphthyridine |
| 2.95 | 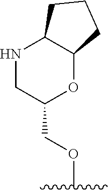 [SMILES: *OC[C@@H]1CN[C@H]2CCC[C@H]2O1] | 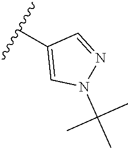 [SMILES: *c1cnn(C(C)(C)C)c1] | 1H NMR (400 MHz, Methanol-d4) δ 8.93 (dd, 1H), 8.61 (ddd, 1H), 8.38 (d, 1H), 8.13 (d, 1H), 7.62 (d, 1H), 7.51 (dd, 1H), 4.78 (dd, 1H), 4.62 (dd, , 1H), 4.33-4.22 (m, 2H), 3.19-3.09 (m, 2H), 2.78 (dd, 1H), 2.10-1.99 (m, 1H), 1.96-1.80 (m, 2H), 1.80-1.70 (m, 1H), 1.66 (s, 9H), 1.64-1.53 (m, 2H). | 1.80 min, [MH]+ = 408 | 2 | 5-{[(2R,4aR,7aS)- octahydrocyclopenta[b][1,4] oxazin-2-yl]methoxy}-7-(1- tert-butyl-1H-pyrazol-4-yl)- 1,6-naphthyridine |
| 2.96 | 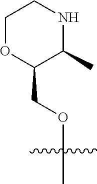 [SMILES: *OC[C@H]1OCCN[C@H]1C] | 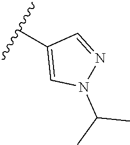 [SMILES: *c1cnn(C(C)C)c1] | 1H NMR (600 MHz, Methanol-d4) δ 8.92 (dd, 1H), 8.58 (ddd, 1H), 8.32 (d, 1H), 8.11 (d, 1H), 7.61 (d, 1H), 7.50 (dd, 1H), 4.68 (dd, 1H), 4.66-4.56 (m, 2H), 4.22 (ddd, 1H), 3.94 (dt, 1H), 3.69-3.63 (m, 1H), 3.23 (qd, 1H), 3.13 (ddd, 1H), 2.70-2.66 (m, 1H), 1.56 (d, 6H), 1.31 (d, 3H). | 1.65 min, [MH]+ = 368 | 2 | 5-{[(2S,3S)-3- methylmorpholin-2- yl]methoxy}-7-[1-(propan-2- yl)-1H-pyrazol-4-yl]-1,6- naphthyridine |
| 2.97 | 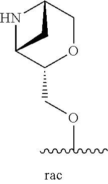 [SMILES: *OC[C@H]1OC[C@@H]2C[C@H]1N2] | 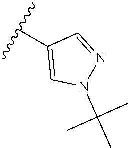 [SMILES: *c1cnn(C(C)(C)C)c1] | 1H NMR (600 MHz, Methanol-d4) δ 8.90 (dd, 1H), 8.58- 8.52 (m, 1H), 8.37 (s, 1H), 8.12 (s, 1H), 7.59 (s, 1H), 7.48 (dd, 1H), 4.83-4.75 (m, 2H), 4.39 (t, 1H), 4.24-4.18 (m, 1H), 4.00 (d, 1H), 3.91-3.85 (m, 1H), 3.75-3.68 (m, 1H), 2.91-2.85 (m, 1H), 1.79 (d, 1H), 1.65 (s, 9H). | 1.69 min, [MH]+ = 380 | 2 | 7-(1-tert-butyl-1H-pyrazol-4- yl)-5-{[rel-(1R,2S,5S)-3-oxa-6- azabicyclo[3.1.1]heptan-2- yl]methoxy}-1,6-naphthyridine- relative stereochemistry tentatively assigned |
| 2.98 | 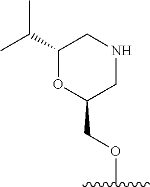 [SMILES: *OC[C@@H]1CNC[C@@H](C(C)C)O1] | 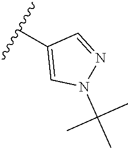 [SMILES: *c1cnn(C(C)(C)C)c1] | 1H NMR (400 MHz, Methanol-d4) δ 8.92 (dd, 1H), 8.58 (ddd, 1H), 8.37 (d, 1H), 8.13 (d, 1H), 7.62 (d, 1H), 7.50 (dd, 1H), 4.68-4.58 (m, 2H), 4.07-3.99 (m, 1H), 3.26 (ddd, 1H), 3.06 (dd, 1H), 2.93 (dd, 1H), 2.68 (dd, 1H), 2.50 (dd, 1H), 1.73-1.63 (m, 10H), 0.97 (d, 3H), 0.92 (d, 3H) | 1.93 min, [MH]+ = 410 | 2 | 7-(1-tert-butyl-1H-pyrazol- 4-yl)-5-{[(2S,6R)-6-(propan- 2-yl)morpholin-2- yl]methoxy}-1,6- naphthyridine |
| 2.99 | 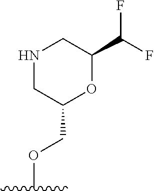 [SMILES: *OC[C@@H]1CNC[C@@H](C(F)F)O1] | 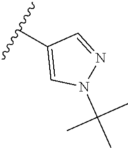 [SMILES: *c1cnn(C(C)(C)C)c1] | 1H NMR (400 MHz, Methanol-d4) δ 8.93 (dd, 1H), 8.59 (ddd, 1H), 8.37 (d, 1H), 8.13 (d, 1H), 7.63 (d, 1H), 7.51 (dd, 1H), 5.81 (td, 1H), 4.72-4.61 (m, 2H), 4.16-4.08 (m, 1H), 3.91-3.79 (m, 1H), 3.15-3.08 (m, 1H), 3.01-2.94 (m, 1H), 2.81-2.69 (m, 2H), 1.66 (s, 9H) | 1.80 min, [MH]+ = 418 | 2 | 7-(1-tert-butyl-1H-pyrazol- 4-yl)-5-{[(2S,6S)-6- (difluoromethyl)morpholin- 2-yl]methoxy}-1,6- naphthyridine |
| 2.100 | 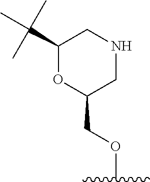 [SMILES: *OC[C@@H]1CNC[C@H](C(C)(C)C)O1] | 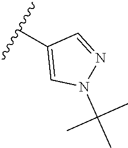 [SMILES: *c1cnn(C(C)(C)C)c1] | 1H NMR (400 MHz, Methanol-d4) δ 8.91 (dd, 1H), 8.61 (ddd, 1H), 8.45 (d, 1H), 8.18 (d, 1H), 7.62 (d, 1H), 7.49 (dd, 1H), 5.61 (dd, 1H), 4.55 (dd, 1H), 4.39-4.28 (m, 1H), 3.60 (dd, 1H), 3.07-2.97 (m, 2H), 2.94 (dd, 1H), 2.62 (dd, 1H), 1.67 (s, 9H), 0.74 (s, 9H) | 1.96 min, [MH]+ = 424 | 2 | 7-(1-tert-butyl-1H-pyrazol- 4-yl)-5-{[(2S,6S)-6-tert- butylmorpholin-2- yl]methoxy}-1,6- naphthyridine |
| 2.101 | 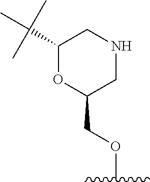 [SMILES: *OC[C@@H]1CNC[C@@H](C(C)(C)C)O1] | 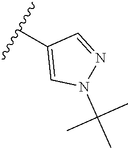 [SMILES: *c1cnn(C(C)(C)C)c1] | 1H NMR (600 MHz, Methanol-d4) δ 8.92 (dd, 1H), 8.56 (ddd, 1H), 8.37 (d, 1H), 8.12 (s, 1H), 7.61 (d, 1H), 7.50 (dd, 1H), 4.70-4.60 (m, 2H), 4.09-4.03 (m, 1H), 3.24 (dd, 1H), 3.11 (dd, 1H), 2.97 (dd, 1H), 2.72 (dd, 1H), 2.61 (dd, 1H), 1.66 (s, 9H), 0.92 (s, 9H) | 2.02 min, [MH]+ = 424 | 2 | 7-(1-tert-butyl-1H-pyrazol- 4-yl)-5-{[(2S,6R)-6-tert- butylmorpholin-2- yl]methoxy}-1,6- naphthyridine |
| 2.102 | 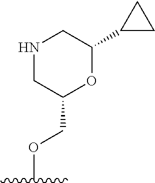 [SMILES: *OC[C@@H]1CNC[C@H](C2CC2)O1] | 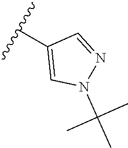 [SMILES: *c1cnn(C(C)(C)C)c1] | 1H NMR (400 MHz, Methanol-d4) δ 8.93 (dd, 1H), 8.61 (ddd, 1H), 8.40 (d, 1H), 8.15 (d, 1H), 7.63 (d, 1H), 7.51 (dd, 1H), 5.18 (dd, 1H), 4.58 (dd, 1H), 4.52-4.43 (m, 1H), 3.29-3.09 (m, 4H), 3.02-2.94 (m, 1H), 1.67 (s, 9H), 1.19 1.06 (m, 1H), 0.57-0.43 (m, 2H), 0.24-0.11 (m, 2H) | 1.80 min, [MH]+ = 408 | 2 | 7-(1-tert-butyl-1H-pyrazol- 4-yl)-5-{[(2S,6S)-6- cyclopropylmorpholin-2- yl]methoxy}-1,6- naphthyridine |
| 2.103 | 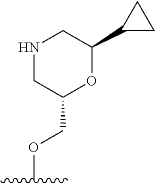 [SMILES: *OC[C@@H]1CNC[C@@H](C2CC2)O1] | 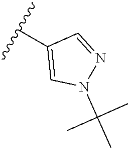 [SMILES: *c1cnn(C(C)(C)C)c1] | 1H NMR (600 MHz, Methanol-d4) δ 8.93 (dd, 1H), 8.60 (ddd, 1H), 8.37 (d, 1H), 8.13 (d, 1H), 7.62 (d, 1H), 7.51 (dd, 1H), 4.69 (dd, 1H), 4.64 (dd, 1H), 4.09-4.01 (m, 1H), 3.16 (dd, 1H), 3.06 (dd, 1H), 2.97 (ddd, 1H), 2.81 (dd, 1H), 2.71 (dd, 1H), 1.66 (s, 9H), 0.92-0.83 (m, 1H), 0.58-0.50 (m, 2H), 0.45-0.37 (m, 1H), 0.34-0.23 (m, 1H) | 1.84 min, [MH]+ = 408 | 2 | 7-(1-tert-butyl-1H-pyrazol- 4-yl)-5-{[(2S,6R)-6- cyclopropylmorpholin-2- yl]methoxy}-1,6- naphthyridine |
| 2.104 | 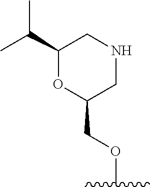 [SMILES: *OC[C@@H]1CNC[C@H](C(C)C)O1] | 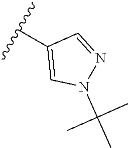 [SMILES: *c1cnn(C(C)(C)C)c1] | 1H NMR (400 MHz, Methanol-d4) δ 8.92 (dd, 1H), 8.60 (ddd, 1H), 8.42 (d, 1H), 8.16 (d, 1H), 7.62 (d, 1H), 7.50 (dd, 1H), 5.23 (dd, 1H), 4.57 (dd, 1H), 4.30-4.21 (m, 1H), 3.53-3.46 (m, 1H), 3.04 (dd, 1H), 2.98-2.87 (m, 2H), 2.70 (dd, 1H), 1.89-1.79 (m, 1H), 1.67 (s, 9H), 0.86-0.77 m, 6H) | 1.91 min, [MH]+ = 410 | 2 | 7-(1-tert-butyl-1H-pyrazol- 4-yl)-5-{[(2S,6S)-6-(propan- 2-yl)morpholin-2- yl]methoxy}-1,6- naphthyridine |
| 2.105 | 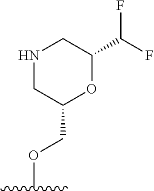 [SMILES: *OC[C@@H]1CNC[C@H](C(F)F)O1] | 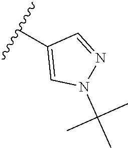 [SMILES: *c1cnn(C(C)(C)C)c1] | 1H NMR (400 MHz, Methanol-d4) δ 8.93 (dd, 1H), 8.60 (ddd, 1H), 8.40 (d, 1H), 8.14 (d, 1H), 7.63 (d, 1H), 7.50 (dd, 1H), 6.01 (td, 1H), 5.03 (dd, 1H), 4.64 (dd, 1H), 4.40- 4.30 (m, 1H), 4.10-3.94 (m, 1H), 3.12 (dd, 1H), 3.07- 2.88 (m, 3H), 1.66 (s, 9H) | 1.83 min, [MH]+ = 418. | 2 | 7-(1-tert-butyl-1H-pyrazol- 4-yl)-5-{[(2S,6R)-6- (difluoromethyl)morpholin- 2-yl]methoxy}-1,6- naphthyridine |
| 2.106 | 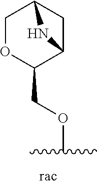 [SMILES: *OC[C@H]1OC[C@@H]2C[C@H]1N2] | 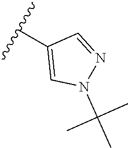 [SMILES: *c1cnn(C(C)(C)C)c1] | 1H NMR (400 MHz, Methanol-d4) δ 8.92 (dd, 1H), 8.57 (d, 1H), 8.37 (s, 1H), 8.12 (s, 1H), 7.62 (s, 1H), 7.50 (dd, 1H), 4.70 (dd, 1H), 4.67-4.60 (m, 1H), 4.60-4.55 (m, 1H), 4.16 (d, 1H), 4.05 (dd, 1H), 3.81-3.73 (m, 1H), 3.68- 3.61 (m, 1H), 2.72-2.64 (m, 1H), 2.14 (d, 1H), 1.66 (s, 9H) | 1.75 min, [MH]+ = 380 | 2 | 7-(1-tert-butyl-1H-pyrazol-4- yl)-5-{[rel-(1R,2S,5S)-3-oxa-6- azabicyclo[3.1.1 ]heptan-2- yl]methoxy}-1,6-naphthyridine- relative stereochemistry tentatively assigned |

TABLE 3
Analytical data for quinolines synthesised by general method B:

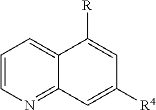

| | | | | LC MS RT, | LC MS | |
|---|---|---|---|---|---|---|
| No | R | R⁴ | 1H NMR | m/z | Method | Name |
| 3.1 | 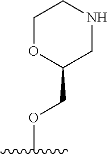 [SMILES: *OC[C@@H]1CNCCO1] | 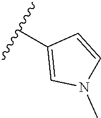 [SMILES: *c1ccn(C)c1] | 1H NMR (400 MHz, Methanol-d4) δ 8.71 (dd, 1H), 8.56 (ddd, 1H), 7.65 (dd, 1H), 7.35 (dd, 1H), 7.29-7.23 (m, 1H), 7.18 (d, 1H), 6.72 (dd, 1H), 6.56 (dd, 1H), 4.25 (dd, 1H), 4.20 (dd, 1H) 4.00 (dddd, 1H), 3.93 (ddd, 1H), 3.76- 3.65 (m, 4H), 3.08 (dd, 1H), 2.93-2.78 (m, 3H). | 1.33 min, [MH]+ = 324 | 2 | 7-(1-methyl-1H- pyrrol-3-yl)-5- {[(2S)-morpholin- 2-yl]methoxy} quinoline |
| 3.2 | 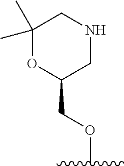 [SMILES: *OC[C@@H]1CNCC(C)(C)O1] | 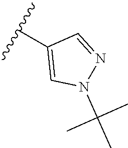 [SMILES: *c1cnn(C(C)(C)C)c1] | 1H NMR (400 MHz, DMSO-d6) δ 8.83 (dd, 1H), 8.57 (d, 1H), 8.41 (ddd, 1H), 8.12 (d, 1H), 7.81 (app t, 1H), 7.45- 7.37 (m, 1H), 7.34 (d, 1H), 4.23-4.16 (m, 1H), 4.13-4.04 (m, 2H), 3.11-3.03 (m, 1H), 2.65-2.58 (m, 1H), 2.46 (d, 2H), 1.59 (s, 9H), 1.32 (s, 3H), 1.10 (s, 3H). | 1.64 min, [MH]+ = 395 | 2 | 7-(1-tert-butyl-1H- pyrazol-4-yl)-5- {[(2S)-6,6- dimethylmorpholin- 2-yl]methoxy} quinoline |
| 3.3 | 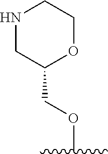 [SMILES: *OC[C@@H]1CNCCO1] | 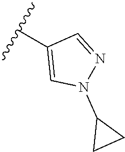 [SMILES: *c1cnn(C2CC2)c1] | 1H NMR (400 MHz, Chloroform-d) δ 8.86 (dd, 1H), 8.51 (ddd, 1H), 7.87 (d, 1H), 7.84 (dd, 1H), 7.78 (dd, 1H), 7.31 (dd, 1H), 6.96 (d, 1H), 4.26 (dd, 1H), 4.15 (dd, 1H), 4.07 (dtd, 1H), 4.04-3.95 (m, 1H), 3.78 (td, 1H), 3.67 (tt, 1H), 3.22 (dd, 1H), 3.06-2.89 (m, 3H), 1.25-1.16 (m, 2H), 1.13-1.02 (m, 2H). | 1.54 min, [MH]+ = 351 | 2 | 7-(1-cyclopropyl- 1H-pyrazol-4-yl)- 5-{[(2S)- morpholin-2- yl]methoxy} quinoline |
| 3.4 | 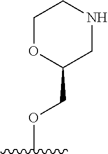 [SMILES: *OC[C@@H]1CNCCO1] | 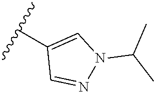 [SMILES: *c1cnn(C(C)C)c1] | 1H NMR (400 MHz, Chloroform-d) δ 8.89-8.81 (m, 1H), 8.54-8.47 (m, 1H), 7.90 (app t, 1H), 7.82-7.79 (m, 2H), 7.31 (ddd, 1H), 6.98 (d, 1H), 4.65-4.48 (m, 1H), 4.27 (dd, 1H), 4.16 (dd, 1H), 4.13-4.03 (m, 1H), 4.04-3.95 (m, 1H), 3.79 (td, 1H), 3.27-3.19 (m, 1H), 3.07-2.87 (m, 3H), 1.58 (dd, 6H). | 1.42 min, [MH]+ = 353 | 2 | 5-{[(2S)-morpholin-2- yl]methoxy}-7-[1-(propan- 2-yl)-1H-pyrazol-4- yl]quinoline |
| 3.5 | 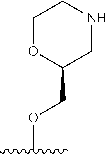 [SMILES: *OC[C@@H]1CNCCO1] | 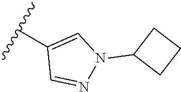 [SMILES: *c1cnn(C2CCC2)c1] | 1H NMR (400 MHz, Chloroform-d) δ 8.85 (dd, 1H), 8.50 (ddd, 1H), 7.91 (d, 1H), 7.82 (d, 1H), 7.78 (dd, 1H), 7.30 (dd, 1H), 6.97 (d, 1H), 4.81 (ttd, 1H), 4.25 (dd, 1H), 4.14 (dd, 1H), 4.04 (dtd, 1H), 3.98 (ddd, 1H), 3.75 (td, 1H) , 3.23-3.16 (m, 1H), 2.98-2.84 (m, 3H), 2.67-2.45 (m, 4H), 2.00-1.80 (m, 2H). | 1.48 min, [MH]+ = 365 | 2 | 7-(1-cyclobutyl-1H- pyrazol-4-yl)-5-{[(2S)- morpholin-2- yl]methoxy}quinoline |
| 3.6 | 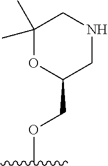 [SMILES: *OC[C@@H]1CNCC(C)(C)O1] | 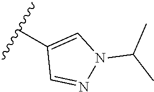 [SMILES: *c1cnn(C(C)C)c1] | 1H NMR (400 MHz, Methanol-d4) δ 8.77 (dd, 1H), 8.59 (ddd, 1H), 8.27 (d, 1H), 8.02 (d, 1H), 7.73 (dd, 1H), 7.42 (dd, 1H), 7.25 (d, 1H), 4.59 (hept, 1H), 4.29-4.13 (m, 3H), 3.09 (ddd, 1H), 2.76-2.58 (m, 3H), 1.55 (d, 6H), 1.38 (s, 3H), 1.19 (s, 3H). | 1.53 min, [MH]+ = 381 | 2 | 5-{[(2S)-6,6- dimethylmorpholin-2- yl]methoxy}-7-[1-(propan- 2-yl)-1H-pyrazol-4- yl]quinoline |
| 3.7 | 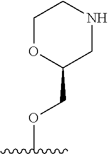 [SMILES: *OC[C@@H]1CNCCO1] | 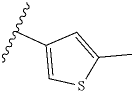 [SMILES: *c1csc(C)c1] | 1H NMR (400 MHz, Methanol-d4) δ 8.79 (dd, 1H), 8.62 (ddd, 1H), 7.77 (app t, 1H), 7.61 (d, 1H), 7.45 (dd, 1H), 7.32-7.26 (m, 2H), 4.31-4.21 (m, 2H), 4.06-3.98 (m, 1H), 3.94 (dd, 1H), 3.72 (ddd, 1H), 3.10 (dd, 1H), 2.94- 2.80 (m, 3H), 2.54 (d, 3H). | 1.62 min, [MH]+ = 341 | 2 | 7-(5-methylthiophen-3-yl)- 5-{[(2S)-morpholin-2- yl]methoxy}quinoline |
| 3.8 | 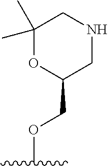 [SMILES: *OC[C@@H]1CNCC(C)(C)O1] | 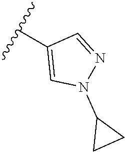 [SMILES: *c1cnn(C2CC2)c1] | 1H NMR (400 MHz, DMSO-d6) δ 8.84 (dd, 1H), 8.52 (d, 1H), 8.41 (ddd, 1H), 8.10 (d, 1H), 7.79 (app t, 1H), 7.42 (dd, 1H), 7.30 (d, 1H), 4.22-4.15 (m, 1H), 4.15-4.02 (m, 2H), 3.81-3.70 (m, 1H), 3.09-3.00 (m, 1H), 2.61 (dd, 1H), 2.48-2.40 (m, 2H), 1.31 (s, 3H), 1.14-1.10 (m, 2H), 1.10 (s, 3H), 1.05-0.96 (m, 2H). | 1.51 min, [MH]+ = 379 | 2 | 7-(1-cyclopropyl-1H- pyrazol-4-yl)-5-{[(2S)-6,6- dimethylmorpholin-2- yl]methoxy}quinoline |
| 3.9 | 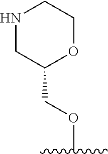 [SMILES: *OC[C@@H]1CNCCO1] | 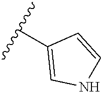 [SMILES: *c1cc[nH]c1] | 1H NMR (400 MHz, Methanol-d4) δ 8.72 (dd, 1H), 8.58 (ddd, 1H), 7.70 (dd, 1H), 7.39-7.35 (m, 2H), 7.24 (d, 1H), 6.84 (dd, 1H), 6.63 (dd, 1H), 4.27 (dd, 1H), 4.22 (dd, 1H), 4.02 (dddd, 1H), 3.95 (ddd, 1H), 3.73 (ddd, 1H), 3.10 (dd, 1H), 2.97-2.79 (m, 3H). | 0.42 min, [MH]+ = 310 | 2 | 5-{[(2S)-morpholin-2- yl]methoxy}-7-(1H-pyrrol- 3-yl)quinoline |
| 3.10 | 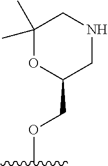 [SMILES: *OC[C@@H]1CNCC(C)(C)O1] | 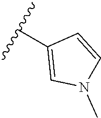 [SMILES: *c1ccn(C)c1] | 1H NMR (400 MHz, Methanol-d4) δ 8.71 (dd, IH), 8.54 (ddd, 1H), 7.65 (dd, 1H), 7.35 (dd, 1H), 7.25 (app t, 1H), 7.19 (d, 1H), 6.75-6.68 (m, 1H), 6.56 (dd, 1H), 4.27- 4.16 (m, 2H), 4.12 (dd, 1H), 3.71 (s, 3H), 3.13-3.04 (m, 1H), 2.74-2.56 (m, 3H), 1.38 (s, 3H), 1.19 (s, 3H). | 1.48 min, [MH]+ = 352 | 2 | 5-{[(2S)-6,6- dimethylmorpholin-2- yl]methoxy}-7-(1-methyl- 1H-pyrrol-3-yl)quinoline |
| 3.11 | 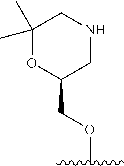 [SMILES: *OC[C@@H]1CNCC(C)(C)O1] | 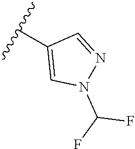 [SMILES: *c1cnn(C(F)F)c1] | 1H NMR (400 MHz, Methanol-d4) δ 8.82 (dd, 1H), 8.67 (d, 1H), 8.62 (ddd, 1H), 8.28 (d, 1H), 7.81 (dd, 1H), 7.55 (t, 1H), 7.47 (dd, 1H), 7.29 (d, 1H), 4.31-4.14 (m, 3H), 3.10 (ddd, 1H), 2.78-2.57 (m, 3H), 1.39 (s, 3H), 1.20 (d, 3H). | 1.60 min, [MH]+ = 389 | 2 | 7-[1-(difluoromethyl)-1H- pyrazol-4-yl]-5-{[(2S)-6,6- dimethylmorpholin-2- yl]methoxy}quinoline |
| 3.12 | 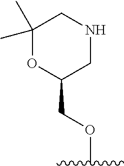 [SMILES: *OC[C@@H]1CNCC(C)(C)O1] | 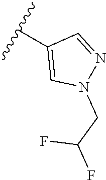 [SMILES: *c1cnn(CC(F)F)c1] | 1H NMR (400 MHz, DMSO-d6) δ 8.85 (dd, 1H), 8.52 (d, 1H), 8.43 (ddd, 1H), 8.24 (d, 1H), 7.80 (app t, 1H), 7.44 (dd, 1H), 7.31 (d, 1H), 6.43 (tt, 1H), 4.67 (td, 2H), 4.27- 4.00 (m, 3H), 3.14-2.96 (m, 1H), 2.61 (d, 1H), 2.48-2.42 (m, 2H), 1.31 (s, 3H), 1.10 (s, 3H). | 1.53 min, [MH]+ = 403 | 2 | 7-[1-(2,2-difluoroethyl)- 1H-pyrazol-4-yl]-5-{[(2S)- 6,6-dimethylmorpholin-2- yl]methoxy}quinoline |
| 3.13 | 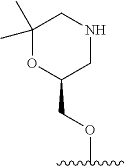 [SMILES: *OC[C@@H]1CNCC(C)(C)O1] | 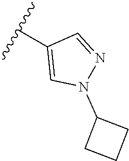 [SMILES: *c1cnn(C2CCC2)c1] | 1H NMR (400 MHz, DMSO-d6) δ 8.84 (dd, 1H), 8.59 (d, 1H), 8.42 (ddd, 1H), 8.14 (d, 1H), 7.79 (app t, 1H), 7.42 (dd, 1H), 7.31 (d, 1H), 4.92-4.78 (m, 1H), 4.24-4.15 (m, 1H), 4.14-4.02 (m, 2H), 3.11-3.00 (m, 1H), 2.65-2.51 (m, 3H), 2.48-2.40 (m, 4H), 1.90-1.75 (m, 2H), 1.31 (s, 3H), 1.10 (s, 3H). | 1.62 min, [MH]+ = 393 | 2 | 7-(1-cyclobutyl-1H- pyrazol-4-yl)-5-{[(2S)-6,6- dimethylmorpholin-2- yl]methoxy}quinoline |
| 3.14 | 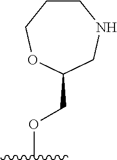 [SMILES: *OC[C@@H]1CNCCCO1] | 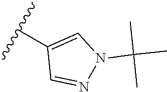 [SMILES: *c1cnn(C(C)(C)C)c1] | 1H NMR (400 MHz, Methanol-d4) δ 8.77 (dd, 1H), 8.60 (ddd, 1H), 8.35 (d, 1H), 8.03 (d, 1H), 7.73 (dd, 1H), 7.41 (dd, 1H), 7.27 (d, 1H), 4.32-4.20 (m, 2H), 4.20-4.11 (m, 1H), 4.06 (ddd, 1H), 3.88-3.80 (m, 1H), 3.27 (dd, 1H), 3.09-2.90 (m, 3H), 2.03-1.85 (m, 2H), 1.65 (s, 9H). | 1.54 min, [MH]+ = 381 | 2 | 7-(1-tert-butyl-1H-pyrazol- 4-yl)-5-{[(2S)-1,4- oxazepan-2- yl]methoxy}quinoline |
| 3.15 | 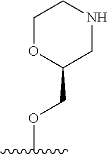 [SMILES: *OC[C@@H]1CNCCO1] | 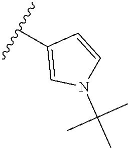 [SMILES: *c1ccn(C(C)(C)C)c1] | 1H NMR (400 MHz, Methanol-d4) δ 8.74-8.68 (m, 1H), 8.56 (app ddt, 1H), 7.69-7.64 (m, 1H), 7.54-7.48 (m, 1H), 7.35 (ddd, 1H), 7.23 (d, 1H), 6.97 (ddd, 1H), 6.58 (ddd, IH), 4.31-4.18 (m, 2H), 4.06-3.96 (m, 1H), 3.98- 3.89 (m, 1H), 3.72 (ddd, 1H), 3.09 (dd, 1H), 2.94-2.72 (m, 3H), 1.59 (s, 9H). | 1.59 min, [MH]+ = 366 | 2 | 7-(1-tert-butyl-1H-pyrrol- 3-yl)-5-{[(2S)-morpholin- 2-yl]methoxy}quinoline |
| 3.16 | 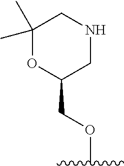 [SMILES: *OC[C@@H]1CNCC(C)(C)O1] | 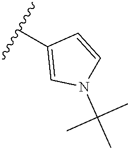 [SMILES: *c1ccn(C(C)(C)C)c1] | 1H NMR (600 MHz, Chloroform-d) δ 8.82 (dd, 1H), 8.44 (ddd, 1H), 7.81 (app t, 1H), 7.27 (app t, 1H), 7.25 (dd, 1H), 7.06 (d, 1H), 6.90 (dd, 1H), 6.60 (dd, 1H), 4.27 (dddd, 1H), 4.23 (dd, 1H), 4.05 (dd, 1H), 3.29 (ddd, 1H), 2.78-2.70 (m, 2H), 2.68 (dd, 1H), 1.60 (s, 9H), 1.41 (s, 3H), 1.24 (s, 3H). | 1.69 min, [MH]+ = 394 | 2 | 7-(1-tert-butyl-1H-pyrrol- 3-yl)-5-{[(28)-6,6- dimethylmorpholin-2- yl]methoxy}quinoline |
| 3.17 | 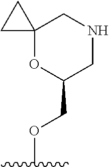 [SMILES: *OC[C@@H]1CNCC2(CC2)O1] | 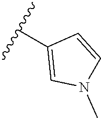 [SMILES: *c1ccn(C)c1] | 1H NMR (600 MHz, Methanol-d4) δ 8.71 (dd, 1H), 8.52 (dd, 1H), 7.65 (s, 1H), 7.35 (dd, 1H), 7.25 (app t, 1H), 7.17 (s, 1H), 6.72 (app t, 1H), 6.56 (app t, 1H), 4.24-4.16 (m, 2H), 4.16-4.10 (m, 1H), 3.72 (s, 3H), 3.33-3.27 (m, 1H), 3.14 (d, 1H), 2.88 (ddd, 1H), 2.35 (d, 1H), 0.94-0.83 (m, 1H), 0.82-0.72 (m, 1H), 0.68-0.56 (m, 2H). | 1.49 min, [MH]+ = 350 | 2 | 7-(1-methyl-1H-pyrrol-3- yl)-5-{[(5S)-4-oxa-7- azaspiro[2.5]octan-5- yl]methoxy}quinoline |
| 3.18 | 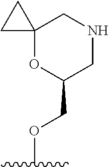 [SMILES: *OC[C@@H]1CNCC2(CC2)O1] | 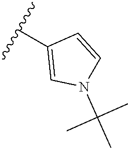 [SMILES: *c1ccn(C(C)(C)C)c1] | 1H NMR (600 MHz, Methanol-d4) δ 8.72 (dd, 1H), 8.54 (dd, 1H), 7.67 (s, 1H), 7.51 (app t, 1H), 7.36 (dd, 1H), 7.23 (s, 1H), 6.98 (app t, 1H), 6.58 (t, 1H), 4.26 (dd, 1H), 4.22 (dd, 1H), 4.19-4.14 (m, 1H), 3.30-3.29 (m, 1H), 3.19- 3.11 (m, 1H), 2.89 (dd, 1H), 2.35 (d, 1H), 1.60 (s, 9H), 0.91-0.85 (m, 1H), 0.82-0.74 (m, 1H), 0.66-0.60 (m, 2H). | 1.71 min, [MH]+ = 392 | 2 | 7-(1-tert-butyl-1H-pyrrol- 3-yl)-5-{[(5S)-4-oxa-7- azaspiro[2.5]octan-5- yl]methoxy}quinoline |
| 3.19 | 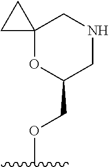 [SMILES: *OC[C@@H]1CNCC2(CC2)O1] | 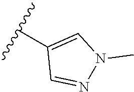 [SMILES: *c1cnn(C)c1] | 1H NMR (600 MHz, Methanol-d4) δ 8.76 (dd, 1H), 8.55 (dd, 1H), 8.14 (s, 1H), 7.99 (s, 1H), 7.69 (s, 1H), 7.41 (dd, 1H), 7.16 (s, 1H), 4.23 (dd, 1H), 4.19 (dd, 1H), 4.17- 4.12 (m, 1H), 3.96 (s, 3H), 3.32-3.28 (m, 1H), 3.17-3.12 (m, 1H), 2.89 (dd, 1H), 2.35 (d, 1H), 0.91-0.83 (m, 1H), 0.82-0.73 (m, 1H), 0.68-0.58 (m, 2H). | 1.46 min, [MH]+ = 351 | 2 | 7-(1-methyl-1H-pyrazol-4- yl)-5-{[(5S)-4-oxa-7- azaspiro[2.5]octan-5- yl]methoxy}quinoline |
| 3.20 | 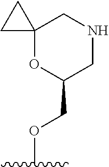 [SMILES: *OC[C@@H]1CNCC2(CC2)O1] | 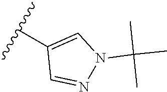 [SMILES: *c1cnn(C(C)(C)C)c1] | 1H NMR (600 MHz, Methanol-d4) δ 8.77 (d, 1H), 8.57 (d, 1H), 8.35 (s, 1H), 8.03 (s, 1H), 7.73 (s, 1H), 7.42 (dd, 1H), 7.25 (s, 1H), 4.27 (dd, 1H), 4.23 (dd, 1H), 4.18- 4.14 (m, 1H), 3.30-3.27 (m, 1H), 3.15 (dd, 1H), 2.92- 2.85 (m, 1H), 2.35 (d, 1H), 1.66 (s, 9H), 0,91-0.83 (m, 1H), 0.83-0.73 (m, 1H), 0.68-0.58 (m, 2H). | 1.67 min, [MH]+ = 393 | 2 | 7-(1-tert-butyl-1H-pyrazol- 4-yl)-5-{[(5S)-4-oxa-7- azaspiro[2.5]octan-5- yl]methoxy}quinoline |
| 3.21 | 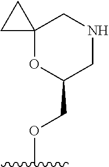 [SMILES: *OC[C@@H]1CNCC2(CC2)O1] | 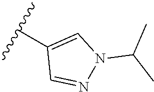 [SMILES: *c1cnn(C(C)C)c1] | 1H NMR (600 MHz, Methanol-d4) δ 8.78 (dd, 1H), 8.57 (dd, 1H), 8.28 (s, 1H), 8.02 (s, 1H), 7.73 (s, 1H), 7.43 (dd, 1H), 7.23 (s, 1H), 4.59 (hept, 1H), 4.26 (dd, 1H), 4.23 (dd, 1H), 4.19-4.14 (m, 1H), 3.32-3.28 (m, 1H), 3.15 (d, 1H), 2.89 (dd, 1H), 2.35 (d, 1H), 1.56 (d, 6H), 0.91-0.83 (m, 1H), 0.82-0.74 (m, 1H), 0,67-0.59 (m, 2H). | 1.60 min, [MH]+ = 379 | 2 | 5-{[(SS)-4-oxa-7- azaspiro[2.5]octan-5- yl]methoxy}-7-[1-(propan- 2-yl)-1H-pyrazol-4- yl]quinoline |
| 3.22 | 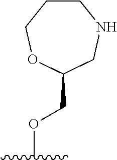 [SMILES: *OC[C@@H]1CNCCCO1] | 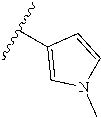 [SMILES: *c1ccn(C)c1] | 1H NMR (400 MHz, Methanol-d4) δ 8.72 (dd, 1H), 8.57 (ddd, 1H), 7.67-7.65 (m, 1H), 7.36 (dd, 1H), 7.27 (t, 1H), 7.20 (d, 1H), 6.76-6.70 (m, 1H), 6.57 (dd, 1H), 4.29- 4.12 (m, 3H), 4.11-4.02 (m, 1H), 3.87-3.78 (m, 1H), 3.73 (s, 3H), 3.27 (dd, 1H), 3.09-2.91 (m, 3H), 2.04-1.87 (m, 2H). | 1.34 min, [MH]+ = 338 | 2 | 7-(1-methyl-1H-pyrrol-3- yl)-5-{[(2S)-1,4-oxazepan- 2-yl]methoxy}quinoline |
| 3.23 | 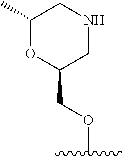 [SMILES: *OC[C@@H]1CNC[C@@H](C)O1] | 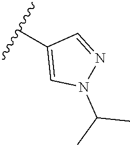 [SMILES: *c1cnn(C(C)C)c1] | 1H NMR (400 MHz, Methanol-d4) δ 8.79 (dd, 1H), 8.63 (ddd, 1H), 8.30 (d, 1H), 8.04 (d, 1H), 7.79-7.75 (m, 1H), 7.44 (dd, 1H), 7.31 (d, 1H), 4.66-4.50 (m, 2H), 4.50- 4.40 (m, 2H), 4.22-4.13 (m, 1H), 3.29-3.13 (m, 3H), 2.83 (dd, 1H), 1.57 (d, 6H), 1.30 (d, 3H) | 1.46 min, [MH]+ = 367 | 2 | 5-{[(2S,6R)-6- methylmorpholin-2- yl]methoxy}-7-[1-(propan- 2-yl)-1H-pyrazol-4- yl]quinoline |
| 3.24 | 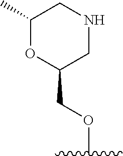 [SMILES: *OC[C@@H]1CNC[C@@H](C)O1] | 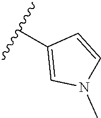 [SMILES: *c1ccn(C)c1] | 1H NMR (400 MHz, Methanol-d4) δ 8.80 (dd, 1H), 8.76- 8.70 (m, 1H), 7.72-7.68 (m, 1H), 7.48 (dd, 1H), 7.37- 7.31 (m, 2H), 6.77 (app t, 1H), 6.61 (dd, 1H), 4.65-4.52 (m, 2H), 4.52-4.44 (m, 1H), 4.38-4.28 (m, 1H), 3.74 (s, 3H), 3.55-3.41 (m, 2H), 3.37 (dd, 1H), 3.08 (dd, 1H), 1.38 (d, 3H). | 1.39 min, [MH]+ = 338 | 2 | 7-(1-methyl-1H-pyrrol-3- yl)-5-{[(2S,6R)-6- methylmorpholin-2- yl]methoxy}quinoline |

TABLE 4
Analytical data for quinazolines synthesised by general method B:

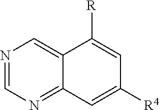

| | | | | LC MS RT, | LC MS | |
|---|---|---|---|---|---|---|
| No | R | R⁴ | 1H NMR | m/z | Method | Name |
| 4.1 | 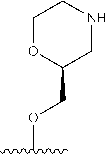 [SMILES: *OC[C@@H]1CNCCO1] | 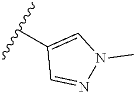 [SMILES: *c1cnn(C)c1] | 1H NMR (400 MHz, Methanol-d4) δ 9.64- 9.60 (m, 1H), 9.14 (s, IH), 8.31-8.27 (m, 1H), 8.10 (d, 1H), 7.72-7.68 (m, 1H), 7.37 (d, 1H), 4.38-4.32 (m, 1H), 4.31 (dd, 1H), 4.08-4.00 (m, 1H), 3.98 (s, 3H), 3.97-3.92 (m, 1H), 3.73 (ddd, 1H), 3.10 (dd, 1H), 2.94-2.80 (m, 3H), | 1.49 min, [MH]+ = 326 | 2 | 7-(1-methyl-1H- pyrazol-4- yl)-5-{[(2S)- morpholin-2- yl]methoxy} quinazoline |
| 4.2 | 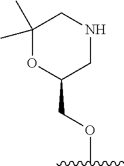 [SMILES: *OC[C@@H]1CNCC(C)(C)O1] | 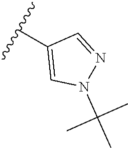 [SMILES: *c1cnn(C(C)(C)C)c1] | 1H NMR (400 MHz, Methanol-d4) δ 9.52 (app t, 1H), 9.10 (s, 1H), 8.42 (d, 1H), 8.08 (d, 1H), 7.65 (app t, 1H), 7.35 (d, 1H), 4.31-4.16 (m, 3H), 3.13-3.05 (m, 1H), 2.73 (dd, IH), 2.69-2.58 (m, 2H), 1.66 (s, 9H), 1.39 (s, 3H), 1.20 (s, 3H). | 1.81 min, [MH]+ = 396 | 2 | 7-(1-tert-butyl- 1H-pyrazol- 4-yl)-5-{[(2S)-6,6- dimethyl- morpholin- 2-yl]methoxy} quinazoline |
| 4.3 | 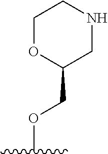 [SMILES: *OC[C@@H]1CNCCO1] | 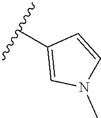 [SMILES: *c1ccn(C)c1] | 1H NMR (600 MHz, Methanol-d4) δ 9.49 (s, 1H), 9.04 (s, 1H), 7.55 (s, 1H), 7.36 (app t, 1H), 7.26 (s, 1H), 6.75 (app t, J = 2.4 Hz, 1H), 6.61 (app t, 1H), 4.30-4.22 (m, 2H), 4.04-3.99 (m, 1H), 3.95 (dd, 1H), 3.78-3.67 (m, 4H), 3.09 (dd, 1H), 2.93-2.80 (m, 3H). | 1.60 min, [MH]+ = 325 | 2 | 7-(1-methyl-1H- pyrrol-3- yl)-5-{[(2S)- morpholin-2- yl]methoxy} quinazoline |

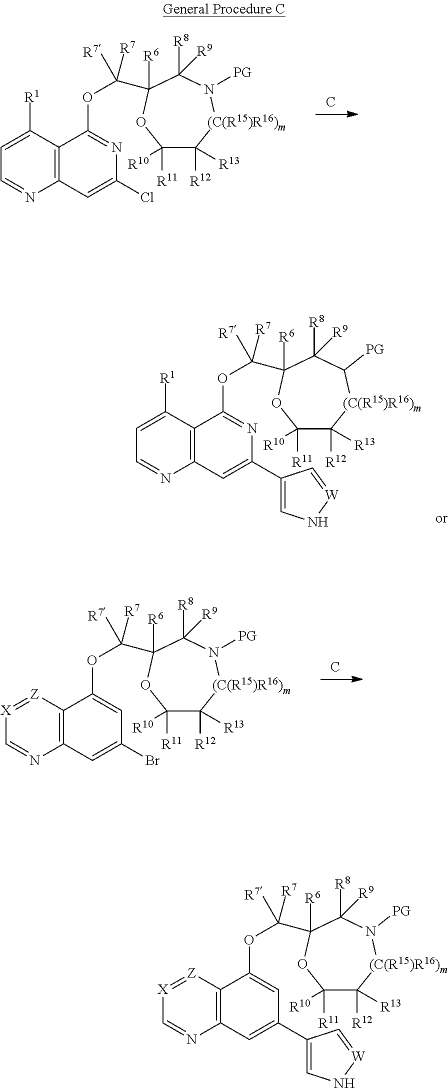

A solution of the appropriate heteroaryl halide (1.0 eq) in 1,4-dioxane/water (0.1-0.4 M, ratio 1:5-1:10) in a microwave vial was degassed with nitrogen for 5 minutes. Sodium carbonate (3.0 eq), tetrakis(triphenylphosphine)palladium (0.1 eq) and the appropriate pinacol boronic ester (1.5 eq) were added. The reaction was heated at 130-135° C. under microwave irradiation for 1 hour. Water was added and the products were extracted with ethyl acetate. The combined organics were washed with brine, dried over anhydrous sodium/magnesium sulfate and concentrated. The crude material was purified using purification method 1.

TABLE 5
Analytical data for intermediates synthesised by general method C

| | | | LC MS RT, | LCMS |
|---|---|---|---|---|
| No | Structure | 1H NMR | m/z | Method |
| 5.1 | 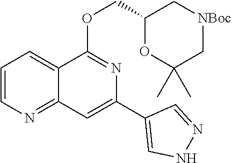 [SMILES: CC(C)(C)OC(=O)N1C[C@@H](COc2nc(-c3cn[nH]c3)cc3ncccc23)OC(C)(C)C1] | 1H NMR (400 MHz, Methanol-d4) δ 8.93 (dd, 1H), 8.66-8.52 (m, 1H), 8.42-8.08 (m, 2H), 7.64 (d, 1H), 7.51 (dd, 1H), 4.65 (dd, 1H), 4.55 (dd, 1H), 4.33-4.13 (m, 2H), 3.80 (dd, 1H), 3.00-2.60 (m, 2H), 1.45 (s, 9H), 1.26 (s, 3H), 1.24 (s, 3H). | 2.72 min, [MH]+ = 440 | 2 |
| 5.2 | 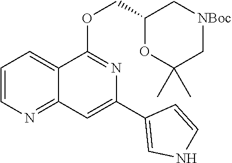 [SMILES: CC(C)(C)OC(=O)N1C[C@@H](COc2nc(-c3cc[nH]c3)cc3ncccc23)OC(C)(C)C1] | 1H NMR (400 MHz, Methanol-d4) δ 8.86 (dd, 1H), 8.60-8.50 (m, 1H), 7.55-7.47 (m, 2H), 7.43 (dd, 1H), 6.81 (dd, 1H), 6.71 (dd, 1H), 4.63 (dd, 1H), 4.53 (dd, 1H), 4.31-4.11 (m, 2H), 3.80 (dd, 1H), 2.94-2.64 (m, 2H), 1.44 (s, 9H), 1.25 (d, 6H). | 2.87 min, [MH]+ = 439 | 2 |
| 5.3 | 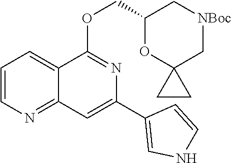 [SMILES: CC(C)(C)OC(=O)N1C[C@@H](COc2nc(-c3cc[nH]c3)cc3ncccc23)OC2(CC2)C1] | 1H NMR (600 MHz, Methanol-d4) δ 8.85 (dd, 1H), 8.53 (d, 1H), 7.52-7.47 (m, 2H), 7.42 (dd, 1H), 6.86-6.79 (m, 1H), 6.73-6.68 (m, 1H), 4.65 (dd, 1H), 4.62-4.54 (m, 1H), 4.31-4.07 (m, 2H), 3.58-3.37 (m, 2H), 3,20- 2.96 (m, 1H), 1.44 (s, 9H), 0.98-0.87 (m, 1H), 0.78-0.68 (m, 2H), 0.64- 0.56 (m, 1H). | 2.78 min, [MH]+ = 437 | 2 |
| 5.4 | 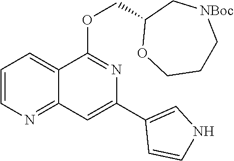 [SMILES: CC(C)(C)OC(=O)N1CCCO[C@H](COc2nc(-c3cc[nH]c3)cc3ncccc23)C1] | 1H NMR (400 MHz, Methanol-d4) mixture of rotamers: δ 8.84 (dd, 1H), 8.57 (d, 0.5H), 8.53 (d, 0.5H), 7.50 (dd, 2H), 7.41 (dd, 1H), 6.81 (t, 1H), 6.70 (dd, 1H), 4.73-4.48 (m, 2H), 4.21-4.08 (m, 2H), 4.02 (dd, 0.5H), 3.95 (dd, 0.5H), 3.75-3.55 (m, 2H), 3.47-3.35 (m, 2H), 1.98-1.83 (m, 2H), 1.48 (s, 4.5H), 1.35 (s, 4.5H). | 2.59 min, [MH]+ = 425 | 2 |
| 5.5 | 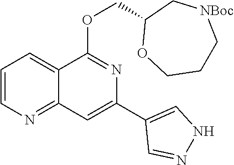 [SMILES: CC(C)(C)OC(=O)N1CCCO[C@H](COc2nc(-c3cn[nH]c3)cc3ncccc23)C1] | 1H NMR (400 MHz, Methanol-d4) mixture of rotamers: δ 8.95-8.88 (m, 1H), 8.64-8.55 (m, 1H), 8.38-8.10 (m, 2H), 7.62 (d, 1H), 7.49 (dd, 1H), 4.75-4.49 (m, 2H), 4.19-4.10 (m, 2H), 4.02 (dd, 0.5H), 3.95 (dd, 0.5H), 3.75-3.55 (m, 2H), 3.50-3.33 (m, 2H), 1.98-1.85 (m, 2H), 1.48 (s, 4.5H), 1.36 (s, 4.5H). | 2.50 min, [MH]+ = 426 | 2 |
| 5.6 | 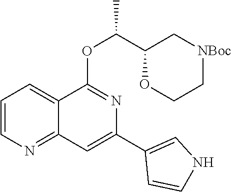 [SMILES: C[C@@H](Oc1nc(-c2cc[nH]c2)cc2ncccc12)[C@@H]1CN(C(=O)OC(C)(C)C)CCO1] | 1H NMR (600 MHz, Methanol-d4) δ 8.86 (dd, J = 4.5, 1.8 Hz, 1H), 8.54 (d, 1H), 7.50 (s, 2H), 7.42 (dd, 1H), 6.82 (app t, 1H), 6.71 (d, 1H), 5.58 (p, 1H), 4.26-4.03 (m, 1H), 3.94 (d, 1H), 3.88-3.76 (m, 1H), 3.70 (ddd, 1H), 3.57 (td, 1H), 3.07-2.97 (m, 1H), 2.97-2.79 (m, 1H), 1.53 (d, 3H), 1.50-1.28 (m, 9H). | 2.73 min, [MH]+ = 425 | 2 |
| 5.7 | 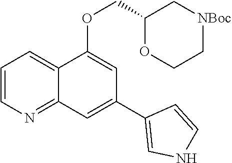 [SMILES: CC(C)(C)OC(=O)N1CCO[C@H](COc2cc(-c3cc[nH]c3)cc3ncccc23)C1] | 1H NMR (400 MHz, Methanol-d4) δ 8.73 (dd, 1H), 8.58 (d, 1H), 7.74-7.69 (m, 1H), 7.40-7.34 (m, 2H), 7.26 (d, 1H), 6.84 (dd, 1H), 6.64 (dd, 1H), 4.34 (dd, 1H), 4.28 (dd, 1H), 4.18 (d, 1H), 4.02-3.85 (m, 3H), 3.64 (td, 1H), 3.16-2.91 (m, 2H), 1.48 (s, 9H). | 2.13 min, [MH]+ = 410 | 2 |
| 5.8 | 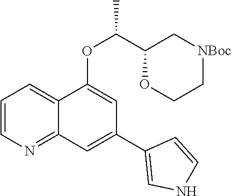 [SMILES: C[C@@H](Oc1cc(-c2cc[nH]c2)cc2ncccc12)[C@@H]1CN(C(=O)OC(C)(C)C)CCO1] | No NMR recorded. | 2.17 min, [MH]+ = 424 | 2 |
| 5.9 | 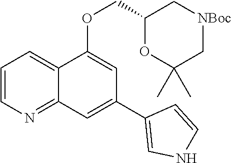 [SMILES: CC(C)(C)OC(=O)N1C[C@@H](COc2cc(-c3cc[nH]c3)cc3ncccc23)OC(C)(C)C1] | 1H NMR (400 MHz, Methanol-d4) δ 8.72 (dd, 1H), 8.64-8.49 (m, 1H), 7.70 (app t, 1H), 7.43-7.31 (m, 2H), 7.25 (d, 1H), 6.83 (dd, 1H), 6.62 (dd, 1H), 4.35-4.13 (m, 4H), 3.82 (d, 1H), 3.04-2.61 (m, 2H), 1.49 (s, 9H), 1.29 (s, 3H), 1.25 (s, 3H). | 2.31 min, [MH]+ = 438 | 2 |
| 5.10 | 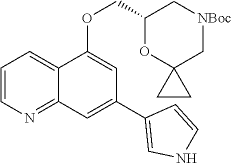 [SMILES: CC(C)(C)OC(=O)N1C[C@@H](COc2cc(-c3cc[nH]c3)cc3ncccc23)OC2(CC2)C1] | 1H NMR (400 MHz, Methanol-d4) δ 8.72 (dd, 1H), 8.55 (d, 1H), 7.72-7.67 (m, 1H), 7.39-7.32 (m, 2H), 7.23 (d, 1H), 6.83 (dd, 1H), 6.62 (dd, 1H), 4.34-4.17 (m, 3H), 4.15-4.07 (m, 1H), 3.63-3.38 (m, 2H), 3.27-2.98 (m, 1H), 1.47 (s, 9H), 0.97-0.88 (m, 1H), 0.83-0.68 (m, 2H), 0.67-0.57 (m, 1H). | 2.27 min, [MH]+ = 436 | 2 |
| 5.11 | 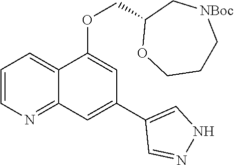 [SMILES: CC(C)(C)OC(=O)N1CCCO[C@H](COc2cc(-c3cn[nH]c3)cc3ncccc23)C1] | 1H NMR (400 MHz, Chloroform-d) mixture of rotamers: δ 8.94-8.81 (m, 1H), 8.60-8.45 (m, 1H), 8.01 (s, 2H), 7.85 (d, 1H), 7.33 (dd, 1H), 7.01 (s, 1H), 4.40-3.99 (m, 5H), 3.89-3.74 (m, 1H), 3.68-3.57 (m, 1H), 3.47- 3.19 (m, 2H), 2.10-1.90 (m, 2H), 1.50 (s, 4.5H), 1.47 (s, 4.5H). | 2.10 min, [MH]+ = 425 | 2 |
| 5.12 | 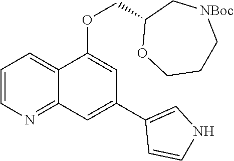 [SMILES: CC(C)(C)OC(=O)N1CCCO[C@H](COc2cc(-c3cc[nH]c3)cc3ncccc23)C1] | 1H NMR (400 MHz, Chloroform-d) mixture of rotamers: δ 8.88-8.80 (m, 1H), 8.66-8.38 (m, 2H), 7.86 (s, 1H), 7.31-7.27 (m, 2H), 7.08 (s, 1H), 6.92-6.87 (m, 1H), 6.72-6.66 (m, 1H), 4.35-4.00 (m, 5H), 3.86-3.74 (m, 1H), 3.70-3.56 (m, 1H), 3.46-3.15 (m, 2H), 2.10-1.90 (m, 2H), 1.50 (s, 4.5H), 1.47 (s, 4.5H). | 2.14 min, [MH]+ = 424 | 2 |
| 5.13 | 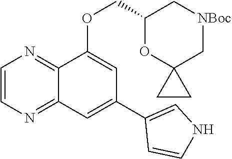 [SMILES: CC(C)(C)OC(=O)N1C[C@@H](COc2cc(-c3cc[nH]c3)cc3nccnc23)OC2(CC2)C1] | 1H NMR (600 MHz, Methanol-d4) δ 8.79 (s, 1H), 8.68 (s, 1H), 7.73 (s, 1H), 7.51 (s, 1H), 7.40 (s, 1H), 6.86 (d, 1H), 6.65 (d, 1H), 4.45-4.27 (m, 2H), 4.25-4.05 (m, 2H), 3.60-3.35 (m, 2H), 3.28-2.99 (m, 1H), 1.46 (s, 9H), 0.99-0.88 (m, 1H), 0.88-0.79 (m, 1H), 0.76-0.65 (m, 1H), 0.65-0.52 (m, 1H). | 2.81 min, [MH]+ = 437 | 2 |
| 5.14 | 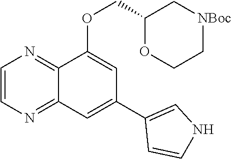 [SMILES: CC(C)(C)OC(=O)N1CCO[C@H](COc2cc(-c3cc[nH]c3)cc3nccnc23)C1] | 1H NMR (400 MHz, Methanol-d4) δ 8.78 (d, 1H), 8.67 (d, 1H), 7.72 (d, 1H), 7.52 (d, 1H), 7.40 (app t, 1H), 6.85 (dd, 1H), 6.65 (dd, 1H), 4.41-4.28 (m, 2H), 4.14 (d, 1H), 4.04-3.90 (m, 2H), 3.90-3.82 (m, 1H), 3.61 (td, 1H), 3.16-2.89 (m, 2H), 1.46 (s, 9H). | 2.56 min, [MH]+ = 411 | 2 |
| 5.15 | 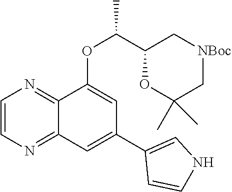 [SMILES: C[C@@H](Oc1cc(-c2cc[nH]c2)cc2nccnc12)[C@@H]1CN(C(=O)OC(C)(C)C)CC(C)(C)O1] | 1H NMR (400 MHz, Methanol-d4) δ 8.77 (d, 1H), 8.68 (d, 1H), 7.73 (d, 1H), 7.59 (s, 1H), 7.39 (app t, 1H), 6.85 (dd, 1H), 6.64 (dd, 1H), 4.74 (br s, 1H), 4.32 (br d, 1H), 4.02-3.89 (m, 1H), 3.75 (d, 1H), 2.80 (dd, 1H), 2.75- 2.57 (m, 1H), 1.47 (d, 3H), 1.44 (s, 9H), 1.25 (s, 3H), 1.18 (br s, 3H). | 2.94 min, [MH]+ = 453 | 2 |
| 5.16 | 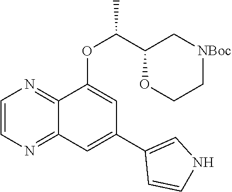 [SMILES: C[C@@H](Oc1cc(-c2cc[nH]c2)cc2nccnc12)[C@@H]1CN(C(=O)OC(C)(C)C)CCO1] | 1H NMR (400 MHz, Methanol-d4) δ 8.78 (d, 1H), 8.67 (d, 1H), 7.73 (d, 1H), 7.58 (s, 1H), 7.40 (d, 1H), 6.85 (app t, 1H), 6.64 (app t, 1H), 4.90-4.79 (m, 1H), 4.36-4,13 (m, 1H), 3.93 (d, 1H), 3.85-3.64 (m, 2H), 3.62-3.53 (m, 1H), 3.17-2.85 (m, 2H), 1.49 (d, 3H), 1.36 (br s, 9H). | 2.66 min, [MH]+ = 425 | 2 |
| 5.17 | 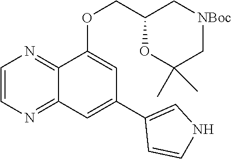 [SMILES: CC(C)(C)OC(=O)N1C[C@@H](COc2cc(-c3cc[nH]c3)cc3nccnc23)OC(C)(C)C1] | 1H NMR (400 MHz, Methanol-d4) δ 8.78 (d, 1H), 8.67 (d, 1H), 7.72 (d, 1H), 7.55-7.54 (m, 1H), 7.39 (app t, 1H), 6.85 (dd, 1H), 6.64 (dd, 1H), 4.38- 4.16 (m, 4H), 3.78 (d, 1H), 3.03-2.55 (m, 2H), 1.46 (s, 9H), 1.28 (s, 3H), 1.22 (s, 3H). | 2.86 min, [MH]+ = 439 | 2 |
| 5.18 | 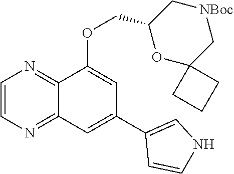 [SMILES: CC(C)(C)OC(=O)N1C[C@@H](COc2cc(-c3cc[nH]c3)cc3nccnc23)OC2(CCC2)C1] | 1H NMR (600 MHz, Methanol-d4) δ 8.73-8.68 (m, 1H), 8.60 (d, 1H), 7.63 (s, 1H), 7.44 (s, 1H), 7.37 (app t, 1H), 6.84 (app t, 1H), 6.61 (dd, 1H), 4.31- 4.24 (m, 1H), 4.24-4.18 (m, 1H), 4.17-4.01 (m, 2H), 4.01-3.94 (m, 1H), 3.03-2.70 (m, 2H), 2.14-2.02 (m, 3H), 1.96-1.86 (m, 1H), 1.86-1.76 (m, 1H), 1.72-1.58 (m, 1H), 1.46 (s, 9H). | 2.855 min, [MH]+ = 451 | 2 |
| 5.19 | 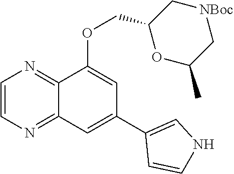 [SMILES: C[C@@H]1CN(C(=O)OC(C)(C)C)C[C@@H](COc2cc(-c3cc[nH]c3)cc3nccnc23)O1] | No 1H NMR recorded | 2.58 min, [MH]+ = 425 | 2 |
| 5.20 | 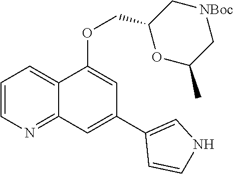 [SMILES: C[C@@H]1CN(C(=O)OC(C)(C)C)C[C@@H](COc2cc(-c3cc[nH]c3)cc3ncccc23)O1] | 1H NMR (400 MHz, Chloroform-d) δ 8.90 (dd, 1H), 8.66 (s, 1H), 7.94 (s, 1H), 7.37-7.32 (m, 1H), 7.32-7.29 (m, 1H), 7.11 (d, 1H), 6.92-6.88 (m, 1H), 6.70-6.66 (m, 1H), 4.41-4.18 (m, 3H), 4.11-3.99 (m, 1H), 3.86- 3.41 (m, 3H), 3.30-2.94 (m, 1H), 1.51-1.28 (m, 9H), 1.27-1.22 (m, 3H). | 2.13 min, [MH]+ = 424 | 2 |

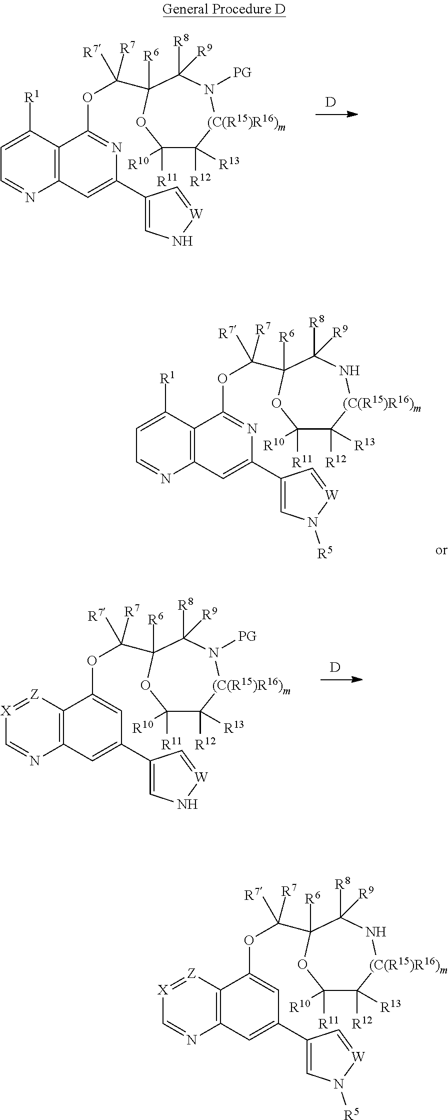

### When Using Alkyl Halide Starting Material:

To a solution of heterocycle starting material (1.0 eq) and the appropriate alkyl halide (5.0-10.0 eq) in N,N-dimethyformamide (0.05-0.3 M) was added sodium hydride (57-63% oil dispersion) (1.0-3.0) eq) and the reaction mixture was stirred at room temperature for 1-24 hours. Water was added and the products were extracted with ethyl acetate. The combined organics were washed with brine, dried over anhydrous sodium/magnesium sulfate and concentrated. The residue was either purified by standard purification method 1, 2 or 3 or taken through crude to the deprotection step.

### When Using Alkyl Tosylates or Alkyl Mesylate Starting Material:

To a solution of heterocycle starting material (1.0 eq) and the appropriate alkyl tosylate or alkyl mesylate (1.0-10.0 eq) in dimethylsulfoxide (0.05-0.3 M) was added cesium carbonate (1.0-3.0 eq). The reaction was heated to 110° C. for 1-24 hours. Water was added and the products were extracted with ethyl acetate. The combined organics were washed with brine, dried over anhydrous sodium/magnesium sulfate and concentrated. The residue was either purified by standard purification method 1, 2 or 3 or taken through crude and the protecting group removed using either of the following conditions:

Boc deprotection conditions 1: To a solution of protected intermediate (1.0 eq) in dichloromethane (0.05-0.2 M) was added trifluoroacetic acid (6-60 eq). The reaction mixture was stirred at room temperature for 1-24 hours. On consumption of starting materials the reaction mixture was purified using one of the standard purification methods.

Boc deprotection conditions 2: A solution of intermediate (1.0 eq) in 1,4-dioxane:water (1:3 ratio, 0.05-0.2 M) was heated at 140-170° C. by microwave irradiation for 1-2 hours. The solvents were removed under reduced pressure and the reaction mixture was purified using one of the standard purification methods.

TABLE 6
Analytical data for naphthyridines synthesised by general method D:

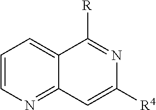

| | | | | LC MS | | |
|---|---|---|---|---|---|---|
| | | | | RT, | LC MS | |
| No | R | R⁴ | 1H NMR | m/z | Method | Name |
| 6.1 | 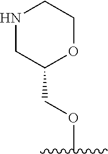 [SMILES: *OC[C@@H]1CNCCO1] | 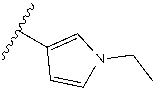 [SMILES: *c1ccn(CC)c1] | 1H NMR (400 MHz, Methanol- d4) δ 8.84 (dd, 1H), 8.53 (ddd, 1H), 7.47 (dd, 1H), 7.45 (d, 2H), 7.40 (dd, 1H), 6.77 (dd, 1H), 6.65 (dd, 1H), 4.63-4.54 (m, 2H), 4.06-3.96 (m, 3H), 3.92 (ddd, 1H), 3.68 (ddd, 1H), 3.07 (dd, 1H), 2.91-2.72 (m, 3H), 1.45 (t, 3H) | 1.61 min, [MH]+ = 339 | 2 | 7-(1-ethyl-1H-pyrrol-3-yl)- 5-{[(2S)-morpholin-2- yl]methoxy}-1,6- naphthyridine |
| 6.2 | 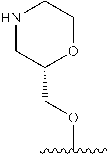 [SMILES: *OC[C@@H]1CNCCO1] | 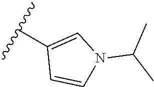 [SMILES: *c1ccn(C(C)C)c1] | 1H NMR (400 MHz, Methanol- d4) δ 8.84 (dd, 1H), 8.54 (ddd, 1H), 7.52 (app t, 1H), 7.46 (d, 1H), 7.41 (dd, 1H), 6.84 (dd, 1H), 6.66 (dd, 1H), 4.64-4.55 (m, 2H), 4.34 (hept, 1H), 4.07-3.96 (m, 1H), 3.92 (ddd, 1H), 3.68 (ddd, 1H), 3.07 (dd, 1H), 2.92- 2.73 (m, 3H), 1.49 (d, 6H). | 1.69 min, [MH]+ = 353 | 2 | 5-{[(2S)-morpholin-2- yl]methoxy}-7-[1-(propan- 2-yl)-1H-pyrrol-3-yl]-1,6- naphthyridine |
| 6.3 | 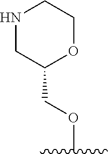 [SMILES: *OC[C@@H]1CNCCO1] | 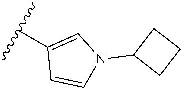 [SMILES: *c1ccn(C2CCC2)c1] | 1H NMR (400 MHz, Methanol- d4) δ 8.85 (dd, 1H), 8.55 (ddd, 1H), 7.55 (app t, 1H), 7.47 (d, 1H), 7.42 (dd, 1H), 6.90-6.84 (m, 1H), 6.67 (dd, 1H), 4.70-4.55 (m, 3H), 4.08-3.98 (m, 1H), 3.96- 3.88 (m, 1H), 3.69 (ddd, 1H), 3.08 (dd, 1H), 2.91-2.74 (m, 3H), 2.54-2.35 (m, 4H), 1.94-1.79 (m, 2H). | 1.79 min, [MH]+ = 365 | 2 | 7-(1-cyclobutyl-1H-pyrrol- 3-yl)-5-{[(2S)-morpholin- 2-yl]methoxy}-1,6- naphthyridine |
| 6.4 | 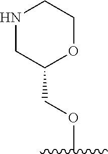 [SMILES: *OC[C@@H]1CNCCO1] | 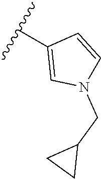 [SMILES: *c1ccn(CC2CC2)c1] | 1H NMR (400 MHz, Methanol- d4) δ 8.84 (dd, 1H), 8.53 (ddd, 1H), 7.53-7.48 (m, 1H), 7.46 (d, 1H), 7.41 (dd, 1H), 6.82 (dd, 1H), 6.66 (dd, 1H), 4.62-4.54 (m, 2H), 4.07-3.97 (m, 1H), 3.96-3.89 (m, 1H), 3.82 (d, 2H), 3.68 (ddd, 1H), 3.09 (dd, 1H), 2.93-2.72 (m, 3H), 1.29- 1.19 (m, 1H), 0.66-0.59 (m, 2H), 0.42-0.34 (m, 2H). | 1.77 min, [MH]+ = 365 | 2 | 7-[1- (cyclopropylmethyl)-1H- pyrrol-3-yl]-5-{[(2S)- morpholin-2- yl]methoxy}-1,6- naphthyridine |
| 6.5 | 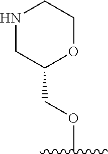 [SMILES: *OC[C@@H]1CNCCO1] | 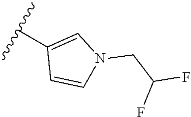 [SMILES: *c1ccn(CC(F)F)c1] | 1H NMR (400 MHz, Methanol- d4) δ 8.86 (dd, 1H), 8.56 (ddd, 1H), 7.56-7.46 (m, 2H), 7.44 (dd, 1H), 6.82 (app t, 1H), 6.72 (dd, 1H), 6.11 (tt, 1H), 4.64-4.55 (m, 2H), 4.37 (td, 2H), 4.06-3.98 (m, 1H), 3.96-3.88 (m, 1H), 3.68 (ddd, 1H), 3.08 (dd, 1H), 2.93-2.72 (m, 3H). | 1.63 min, [MH]+ = 375 | 2 | 7-[1-(2,2-difluoroethyl)- 1H-pyrrol-3-yl]-5-{[(2S)- morpholin-2-yl]methoxy}- 1,6-naphthyridine |
| 6.6 | 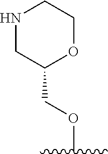 [SMILES: *OC[C@@H]1CNCCO1] | 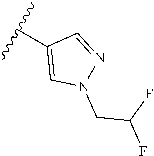 [SMILES: *c1cnn(CC(F)F)c1] | 1H NMR (400 MHz, Methanol- d4) δ 8.93 (dd, 1H), 8.60 (ddd, 1H), 8.35 (s, 1H), 8.17 (d, 1H), 7.62 (d, 1H), 7.52 (dd, 1H), 6.24 (tt, 1H), 4.70- 4.55 (m, 4H), 4.06-3.98 (m, 1H), 3,96-3.86 (m, 1H), 3.73-3.64 (m, 1H), 3.07 (dd, 1H), 2.93-2.73 (m, 3H). | 1.59 min, [MH]+ = 376 | 2 | 7-[1-(2,2-difluoroethyl)- 1H-pyrazol-4-yl]-5- {[(2S)-morpholin-2- yl]methoxy}-1,6- naphthyridine |
| 6.7 | 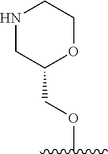 [SMILES: *OC[C@@H]1CNCCO1] | 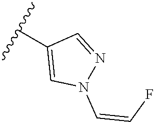 [SMILES: *c1cnn(/C=C\F)c1] | 1H NMR (400 MHz, Methanol- d4) δ 8.92 (dd, 1H), 8.56 (ddd, 1H), 8.53 (app t, 1H), 8.20 (s, 1H), 7.61 (d, 1H), 7.50 (dd, 1H), 6.83 (dd, 1H), 6.73-6.65 (m, 1H), 4.58 (d, 2H), 4.09-3.99 (m, 1H), 3.94 (ddd, 1H), 3.70 (ddd, 1H), 3.11 (dd, 1H), 2.96- 2.76 (m, 3H). | 1.63 min, [MH]+ = 356 | 2 | 7-{1-[(1Z)-2- fluoroethenyl]-1H- pyrazol-4-yl}-5-{[(2S)- morpholin-2- yl]methoxy}-1,6- naphthyridine |
| 6.8 | 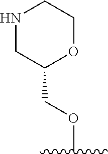 [SMILES: *OC[C@@H]1CNCCO1] | 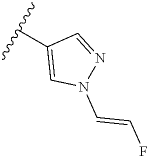 [SMILES: *c1cnn(/C=C/F)c1] | 1H NMR (400 MHz, Methanol- d4) δ 8.93 (dd, 1H), 8.58 (ddd, 1H), 8.36 (d, 1H), 8.20 (s, 1H), 7.80 (dd, 1H), 7.61 (d, 1H), 7.51 (dd, 1H), 7.46 (dd, 1H), 4.64 (d, 2H), 4.16- 4.05 (m, 1H), 4.05-3.96 (m, 1H), 3.83-3.69 (m, 1H), 3.23 (dd, 1H), 3.02-2.87 (m, 3H). | 1.66 min, [MH]+ = 356 | 2 | 7-{1-[(1E)-2- fluoroethenyl]-1H- pyrazol-4-yl}-5-{[(2S)- morpholin-2- yl]methoxy}-1,6- naphthyridine |
| 6.9 | 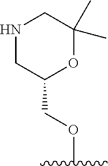 [SMILES: *OC[C@@H]1CNCC(C)(C)O1] | 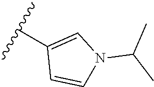 [SMILES: *c1ccn(C(C)C)c1] | 1H NMR (400 MHz, Methanol- d4) δ 8.84 (dd, 1H), 8.52 (ddd, 1H), 7.52 (app t, 1H), 7.45 (d, 1H), 7.40 (dd, 1H), 6.83 (dd, 1H), 6.65 (dd, 1H), 4.57 (dd, 1H), 4.46 (dd, 1H), 4.39-4.30 (m, 1H), 4.30- 4.22 (m, 1H), 3.08 (ddd, 1H), 2.71 (dd, 1H), 2.60 (ddd, 2H), 1.49 (d, 1.36 (s, 3H), 1.19 (s, 3H). | 1.85 min, [MH]+ = 381 | 2 | 5-{[(2S)-6,6- dimethylmorpholin-2- yl]methoxy}-7-[1- (propan-2-yl)-1H-pyrrol- 3-yl]-1,6-naphthyridine |
| 6.10 | 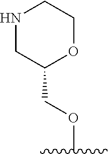 [SMILES: *OC[C@@H]1CNCCO1] | 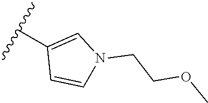 [SMILES: *c1ccn(CCOC)c1] | 1H NMR (400 MHz, Methanol- d4) δ 8.84 (dd, 1H), 8.54 (ddd, 1H), 7.48 (app t, 1H), 7.46 (d, 1H), 7.41 (dd, 1H), 6.79 (dd, 1H), 6.65 (dd, 1H), 4.59 (d, 2H), 4.11 (t, 2H), 4.06-3.98 (m, 1H), 3.92 (dd, 1H), 3.74-3.63 (m, 3H), 3.34 (s, 3H), 3.08 (dd, 1H), 2.92-2.74 (m, 3H). | 1.57 min, [MH]+ = 369 | 2 | 7-[1-(2-methoxyethyl)- 1H-pyrrol-3-yl]-5- {[(2S)-morpholin-2- yl]methoxy}-1,6- naphthyridine |
| 6.11 | 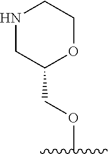 [SMILES: *OC[C@@H]1CNCCO1] | 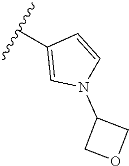 [SMILES: *c1ccn(C2COC2)c1] | 1H NMR (400 MHz, Methanol- d4) δ 8.86 (dd, 1H), 8.58- 8.51 (m, 1H), 7.74 (app t, 1H), 7.50 (s, 1H), 7.43 (dd, 1H), 7.06 (app t, 1H), 6.76 (dd, 1H), 5.38 (tt, 1H), 5.14-5.06 (m, 2H), 4,96-4.88 (m, 2H), 4.66-4.54 (m, 2H), 4.05- 3.98 (m, 1H), 3.92 (ddd, 1H), 3.72-3.65 (m, 1H), 3.08 (dd, 1H), 2.93-2.74 (m, 3H). | 1.55 min, [MH]+ = 367 | 2 | 5-{[(2S)-morpholin-2- yl]methoxy}-7-[1- (oxetan-3-yl)-1H-pyrrol- 3-yl]-1,6-naphthyridine |
| 6.12 | 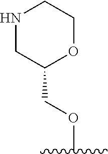 [SMILES: *OC[C@@H]1CNCCO1] | 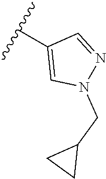 [SMILES: *c1cnn(CC2CC2)c1] | 1H NMR (400 MHz, Methanol- d4) δ 8.92 (dd, 1H), 8.60 (ddd, 1H), 8.32 (d, 1H), 8.10 (d, 1H), 7.60 (d, 1H), 7.50 (dd, 1H), 4.61 (d, 2H), 4.06 (d, 2H), 4.02 (ddd, 1H), 3.96 3.88 (m, 1H), 3.75-3.63 (m, 1H), 3.14-3.04 (m, 1H), 2.94-2.73 (m, 3H), 1.42-1.29 (m, 1H), 0.70-0.61 (m, 2H), 0.49-0.41 (m, 2H). | 1.68 min, [MH]+ = 366 | 2 | 7-[1- (cyclopropylmethyl)-1H- pyrazol-4-yl]-5-{[(2S)- morpholin-2- yl]methoxy}-1,6- naphthyridine |
| 6.13 | 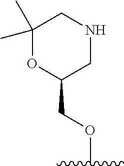 [SMILES: *OC[C@@H]1CNCC(C)(C)O1] | 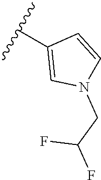 [SMILES: *c1ccn(CC(F)F)c1] | 1H NMR (400 MHz, Methanol- d4) δ 8.86 (dd, 1H), 8.54 (ddd, 1H), 7.52 (app t, 1H), 7.49 (d, 1H), 7.44 (dd, 1H), 6.82 (app t, 1H), 6.73 (dd, 1H), 6.11 (tt, 1H), 4.62-4.44 (m, 2H), 4.37 (td, 2H), 4.31- 4.21 (m, 1H), 3.08 (dd, 1H), 2.71 (d, 1H), 2.66-2.54 (m, 2H), 1.36 (s, 3H), 1.19 (s, 3H) | 1.75 min, [MH]+ = 403 | 2 | 7-[1-(2,2-difluoroethyl)- 1H-pyrrol-3-yl]-5- {[(2S)-6,6- dimethylmorpholin-2- yl]methoxy}-1,6- naphthyridine |
| 6.14 | 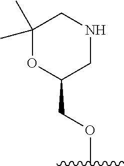 [SMILES: *OC[C@@H]1CNCC(C)(C)O1] | 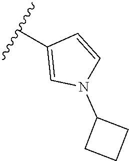 [SMILES: *c1ccn(C2CCC2)c1] | 1H NMR (400 MHz, Methanol- d4) δ 8.85 (dd, 1H), 8.53 (ddd, 1H), 7.55 (app t, 1H), 7.46 (d, 1H), 7.42 (dd, 1H), 6.91-6.82 (m, 1H), 6.67 (dd, 1H), 4.70-4.55 (m, 2H), 4.47 (dd, 1H), 4.34-4.22 (m, 1H), 3.16-3.05 (m, 1H), 2.73 (d, 1H), 2.68-2.57 (m, 2H), 2.56-2.35 (m, 4H), 1.93-1.80 (m, 2H), 1.37 (s, 3H), 1.20 (s, 3H). | 1.93 min, [MH]+ = 393 | 2 | 7-(1-cyclobutyl-1H- pyrrol-3-yl)-5-{[(2S)- 6,6-dimethylmorpholin- 2-yl]methoxy}-1,6- naphthyridine |
| 6.15 | 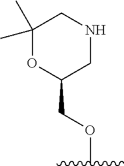 [SMILES: *OC[C@@H]1CNCC(C)(C)O1] | 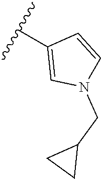 [SMILES: *c1ccn(CC2CC2)c1] | 1H NMR (400 MHz, Methanol- d4) δ 8.82 (dd, 1H), 8.48 (ddd, 1H), 7.49 (app t, 1H), 7.43 (d, 1H), 7.38 (dd, 1H), 6.81 (dd, , 1H), 6.65 (dd, 1H), 4.54 (dd, 1H), 4.45 (dd, 1H), 4.29-4.20 (m, 1H), 3.80 (d, 2H), 3.07 (ddd, 1H), 2.71 (dd, 1H), 2.65-2.55 (m, 2H), 1.35 (s, 3H), 1.32- 1.21 (m, 1H), 1.21 (s, 3H), 0.69-0.54 (m, 2H), 0.45- 0.29 (m, 2H). | 1.90 min, [MH]+ = 393 | 2 | 7-[1- (cyclopropylmethyl)-1H- pyrrol-3-yl]-5-{[(2S)- 6,6-dimethylmorpholin- 2-yl]methoxy}-1,6- naphthyridine |
| 6.16 | 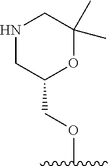 [SMILES: *OC[C@@H]1CNCC(C)(C)O1] | 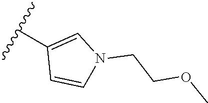 [SMILES: *c1ccn(CCOC)c1] | 1H NMR (400 MHz, Methanol- d4) δ 8.81 (dd, 1H), 8.48 (ddd, 1H), 7.46 (app t, 1H), 7.42 (d, 1H), 7.38 (dd, 1H), 6.78 (dd, 1H), 6.64 (dd, 1H), 4.53 (dd, 1H), 4.44 (dd, 1H), 4.30-4.18 (m, 1H), 4.10 (t, 2H), 3.68 (t, 2H), 3.33 (s, 3H), 3.07 (ddd, 1H), 2.77- 2.66 (m, 1H), 2.66-2.52 (m, 2H), 1.35 (s, 3H), 1.19 (s, 3H). | 1.70 min, [MH]+ = 397 | 2 | 5-{[(2S)-6,6- dimethylmorpholin-2- yl]methoxy}-7-[1-(2- methoxyethyl)-1H- pyrrol-3-yl]-1,6- naphthyridine |
| 6.17 | 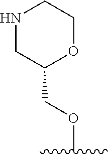 [SMILES: *OC[C@@H]1CNCCO1] | 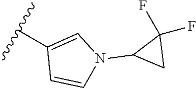 [SMILES: *c1ccn(C2CC2(F)F)c1] | 1H NMR (400 MHz, Methanol- d4) δ 8.86 (dd, 1H), 8.55 (ddd, 1H), 7.52 (app t, 1H), 7.49 (d, 1H), 7.44 (dd, 1H), 6.85 (app td, 1H), 6.72 (dd, 1H), 4.66-4.55 (m, 2H), 4.18- 4.08 (m, 1H), 4.07-3.99 (m, 1H), 3.98-3.90 (m, 1H), 3.70 (ddd, 1H), 3.12 (dd, 1H), 2.96-2.75 (m, 3H), 2.23- 2.04 (m, 2H). | 1.74 min, [MH]+ = 387 | 2 | 7-[1-(2,2- difluorocyclopropyl)- 1H-pyrrol-3-yl]-5- {[(2S)-morpholin-2- yl]methoxy}-1,6- naphthyridine |
| 6.18 | 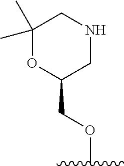 [SMILES: *OC[C@@H]1CNCC(C)(C)O1] | 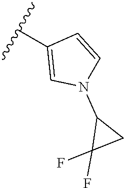 [SMILES: *c1ccn(C2CC2(F)F)c1] | 1H NMR (400 MHz, Methanol- d4) δ 8.86 (dd, 1H), 8.53 (ddd, 1H), 7.52 (app t, 1H), 7.48 (d, 1H), 7.44 (dd, 1H), 6.87-6.82 (m, 1H), 6.72 (dd, 1H), 4.58 (ddd, 1H), 4.47 (ddd, 1H), 4.32-4.22 (m, 1H), 4.17-4.07 (m, 1H), 3.15- 3.04 (m, 1H), 2.73 (d, 1H), 2.68-2.57 (m, 2H), 2.22- 2.04 (m, 2H), 1.36 (s, 3H), 1.20 (s, 3H). | 1.87 min, [MH]+ = 415 | 2 | 7-[1-(2,2- difluorocyclopropyl)- 1H-pyrrol-3-yl]-5- {[(2S)-6,6- dimethylmorpholin-2- yl]methoxy}-1,6- naphthyridine |
| 6.19 | 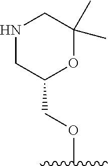 [SMILES: *OC[C@@H]1CNCC(C)(C)O1] | 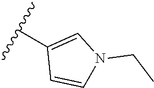 [SMILES: *c1ccn(CC)c1] | 1H NMR (400 MHz, Methanol- d4) δ 8.83 (dd, 1H), 8.51 (ddd, 1H), 7.47 (app t, 1H), 7.44 (d, 1H), 7.40 (dd, 1H), 6.77 (dd, 1H), 6.65 (dd, 1H), 4.55 (dd, 1H), 4.46 (dd, 1H), 4.30-4.20 (m, 1H), 4.00 (q, 2H), 3.07 (ddd, 1H), 2.70 (dd, 1H), 2.64-2.54 (m, 2H), 1.45 (t, 3H), 1.36 (s, 3H), 1.19 (s, 3H). | 1.79 min, [MH]+ = 367 | 2 | 5-{[(2S)-6,6- dimethylmorpholin-2- yl]methoxy}-7-(1-ethyl- 1H-pyrrol-3-yl)-1,6- naphthyridine |
| 6.20 | 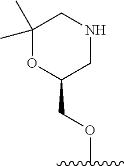 [SMILES: *OC[C@@H]1CNCC(C)(C)O1] | 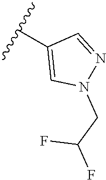 [SMILES: *c1cnn(CC(F)F)c1] | 1H NMR (400 MHz, Methanol- d4) δ 8.93 (dd, 1H), 8.58 (ddd, 1H), 8.34 (d, 1H), 8.17 (d, 1H), 7.61 (d, 1H), 7.51 (dd, 1H), 6.24 (tt, 1H), 4.73- 4.43 (m, 4H), 4.32-4.19 (m, 1H), 3.08 (ddd, 1H), 2.72 (dd, 1H), 2.68-2.56 (m, 2H), 1.36 (s, 3H), 1.19 (s, 3H). | 1.72 min, [MH]+ = 404 | 2 | 7-[1-(2,2-difluoroethyl)- 1H-pyrazol-4-yl]-5- {[(2S)-6,6- dimethylmorpholin-2- yl]methoxy}-1,6- naphthyridine |
| 6.21 | 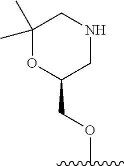 [SMILES: *OC[C@@H]1CNCC(C)(C)O1] | 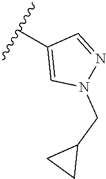 [SMILES: *c1cnn(CC2CC2)c1] | 1H NMR (400 MHz, Methanol- d4) δ 8.92 (dd, 1H), 8.58 (ddd, 1H), 8.32 (d, 1H), 8.10 (d, 1H), 7.60 (d, 1H), 7.50 (dd, 1H), 4.58 (dd, 1H), 4.50 (dd, 1H), 4.33-4.19 (m, 1H), 4.06 (d, 2H), 3.08 (ddd, 1H), 2.77-2.68 (m, 1H), 2.67-2.54 (m, 2H), 1.43- 1.30 (m, 4H), 1.19 (s, 3H), 0.70-0.60 (m, 2H), 0.49-0.42 (m, 2H). | 1.80 min, [MH]+ = 394 | 2 | 7-[1- (cyclopropylmethyl)-1H- pyrazol-4-yl]-5-{[(2S)- 6,6-dimethylmorpholin- 2-yl]methoxy}-1,6- naphthyridine |
| 6.22 | 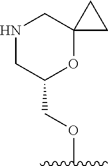 [SMILES: *OC[C@@H]1CNCC2(CC2)O1] | 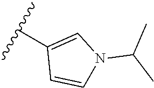 [SMILES: *c1ccn(C(C)C)c1] | 1H NMR (400 MHz, Methanol- d4) δ 8.84 (dd, 1H), 8.50 (ddd, 1H), 7.52 (app t, 1H), 7.45 (d, 1H), 7.40 (dd, 1H), 6.84 (dd, 1H), 6.66 (dd, 1H), 4.60 (dd, 1H), 4.53 (dd, 1H), 4.34 (hept, 1H), 4.20-4.13 (m, 1H), 3.35-3.22 (m, 1H), 3.14 (ddd, 1H), 2.84 (dd, 1H), 2.32 (dd, 1H), 1.50 (d, 6H), 0.94-0.82 (m, 1H), 0.81- 0.69 (m, 1H), 0.66-0.57 (m, 2H). | 1.82 min, [MH]+ = 379 | 2 | 5-{[(5S)-4-oxa-7- azaspiro[2.5]octan-5- yl]methoxy}-7-[1- (propan-2-yl)-1H-pyrrol- 3-yl]-1,6-naphthyridine |
| 6.23 | 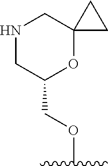 [SMILES: *OC[C@@H]1CNCC2(CC2)O1] | 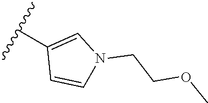 [SMILES: *c1ccn(CCOC)c1] | 1H NMR (400 MHz, Methanol- d4) δ 8.85 (dd, 1H), 8.51 (ddd, 1H), 7.48 (dd, 1H), 7.46 (d, 1H), 7.41 (dd, 1H), 6.80 (dd, 1H), 6.66 (dd, 1H), 4.59 (dd, 1H), 4.54 (dd, 1H), 4.20- 4.14 (m, 1H), 4.12 (t, 2H), 3.70 (t, 2H), 3.35 (s, 3H), 3.32-3.25 (m, 1H), 3.14 (ddd, 1H), 2.83 (dd, 1H), 2.32 (dd, 1H), 0.95-0.82 (m, 1H), 0.81-0.70 (m, 1H), 0.66- 0.57 (m, 2H). | 1.68 min, [MH]+ = 395 | 2 | 7-[1-(2-methoxyethyl)- 1H-pyrrol-3-yl]-5- {[(5S)-4-oxa-7- azaspiro[2.5]octan-5- yl]methoxy}-1,6- naphthyridine |
| 6.24 | 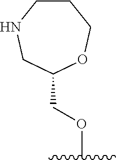 [SMILES: *OC[C@@H]1CNCCCO1] | 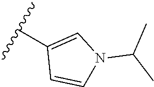 [SMILES: *c1ccn(C(C)C)c1] | 1H NMR (400 MHz, Methanol- d4) δ 8.84 (dd, 1H), 8.54 (ddd, 1H), 7.53 (app t, 1H), 7.46 (d, 1H), 7.41 (dd, 1H), 6.84 (dd, 1H), 6.66 (dd, 1H), 4.63 (dd, 1H), 4.54 (dd, 1H), 4.34 (hept, 1H), 4.17 (dddd, 1H), 4.05 (ddd, 1H), 3.80 (ddd, 1H), 3.25 (dd, 1H), 3.03 (ddd, 1H), 2.96-2.88 (m, 2H), 2.01-1.83 (m, 2H), 1.49 (d, 6H). | 1.74 min [MH]+ = 367 | 2 | 5-{[(2S)-1,4-oxazepan- 2-yl]methoxy}-7-[1- (propan-2-yl)-1H-pyrrol- 3-yl]-1,6-naphthyridine |
| 6.25 | 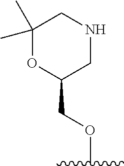 [SMILES: *OC[C@@H]1CNCC(C)(C)O1] | 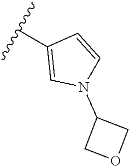 [SMILES: *c1ccn(C2COC2)c1] | 1H NMR (400 MHz, Methanol- d4) δ 8.86 (dd, 1H), 8.53 (ddd, 1H), 7.75 (dd, 1H), 7.50 (d, 1H), 7.43 (dd, 1H), 7.06 (dd, 1H), 6.76 (dd, 1H), 5.43- 5.32 (m, 1H), 5.10 (t, 2H), 4.96-4.88 (m, 2H), 4.58 (dd, 1H), 4.47 (dd, 1H), 4.31- 4.21 (m, 1H), 3.08 (ddd, 1H), 2.70 (dd, 1H), 2.65-2.55 (m, 2H), 1.36 (s, 3H), 1.19 (s, 3H). | 1.65 min, [MH]+ = 395 | 2 | 5-{[(2S)-6,6- dimethylmorpholin-2- yl]methoxy}-7-[1- (oxetan-3-yl)-1H-pyrrol- 3-yl]-1,6-naphthyridine |
| 6.26 | 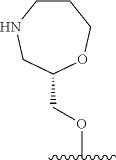 [SMILES: *OC[C@@H]1CNCCCO1] | 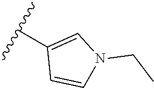 [SMILES: *c1ccn(CC)c1] | 1H NMR (400 MHz, Methanol- d4) δ 8.84 (dd, 1H), 8.54 (ddd, 1H), 7.49-7.46 (m, 1H), 7.45 (d, 1H), 7.41 (dd, 1H), 6.77 (dd, 1H), 6.65 (dd, 1H), 4.62 (dd, 1H), 4.54 (dd, 1H), 4.15 (dddd, 1H), 4.09- 3.96 (m, 3H), 3.79 (ddd, 1H), 3.24 (dd, 1H), 3.01 (ddd, 1H), 2.95-2.82 (m, 2H), 2.02- 1.82 (m, 2H), 1.45 (t, 3H). | 1.64 min, [MH]+ = 353 | 2 | 7-(1-ethyl-1H-pyrrol-3- yl)-5-{[(2S)-1,4- oxazepan-2- yl]methoxy}-1,6- naphthyridine |
| 6.27 | 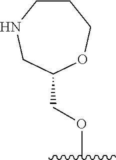 [SMILES: *OC[C@@H]1CNCCCO1] | 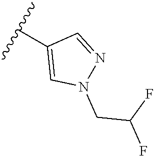 [SMILES: *c1cnn(CC(F)F)c1] | 1H NMR (400 MHz, Methanol- d4) δ 8.93 (dd, 1H), 8.61 (ddd, 1H), 8.35 (d, 1H), 8.18 (d, 1H), 7.63 (d, 1H), 7.52 (dd, 1H), 6.24 (tt, 1H), 4.70- 4.52 (m, 4H), 4.17 (dddd, 1H), 4.05 (ddd, 1H), 3.80 (ddd, 1H), 3.25 (dd, 1H), 3.03 (ddd, 1H), 2.97-2.88 (m, 2H), 2.01-1.83 (m, 2H). | 1.61 min, [MH]+ = 390 | 2 | 7-[1-(2,2-difluoroethyl)- 1H-pyrazol-4-yl]-5- {[(2S)-1,4-oxazepan-2- yl]methoxy}-1,6- naphthyridine |
| 6.28 | 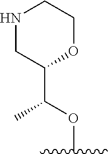 [SMILES: *O[C@H](C)[C@@H]1CNCCO1] | 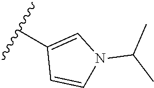 [SMILES: *c1ccn(C(C)C)c1] | 1H NMR (400 MHz, Methanol- d4) δ 8.81 (dd, 1H), 8.47 (ddd, 1H), 7.49 (app t, 1H), 7.43 (d, 1H), 7.37 (dd, 1H), 6.82 (dd, 1H), 6.65 (dd, 1H), 5.59-5.48 (m, 1H), 4.31 (hept, 1H), 3.89 (ddd, 1H), 3.74 (ddd, 1H), 3.63 (ddd, 1H), 3.07 (dd, 1H), 2.84-2.70 (m, 3H), 1.49-1.45 (m, 9H). | 1.78 min, [MH]+ = 367 | 2 | 5-[(1R)-1-[(2S)- morpholin-2-yl]ethoxy]- 7-[1-(propan-2-yl)-1H- pyrrol-3-yl]-1,6- naphthyridine |
| 6.29 | 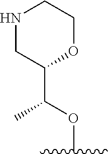 [SMILES: *O[C@H](C)[C@@H]1CNCCO1] | 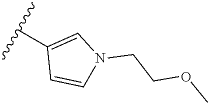 [SMILES: *c1ccn(CCOC)c1] | 1H NMR (400 MHz, Methanol- d4) δ 8.82 (dd, 1H), 8.49 (ddd, 1H), 7.47-7.44 (m, 1H), 7.43 (d, 1H), 7.38 (dd, 1H), 6.78 (dd, 1H), 6.64 (dd, 1H), 5.59-5.48 (m, 1H), 4.10 (t, 2H), 3.94-3.86 (m, 1H), 3.74 (ddd, 1H), 3.70- 3.65 (m, 2H), 3.65-3.59 (m, 1H), 3.34 (s, 3H), 3.08 (dd, 1H), 2.87-2.68 (m, 3H), 1.47 (d, 3H). | 1.65 min, [MH]+ = 383 | 2 | 7-[1-(2-methoxyethyl)- 1H-pyrrol-3-yl]-5-[(1R)- 1-[(2S)-morpholin-2- yl]ethoxy]-1,6- naphthyridine |
| 6.30 | 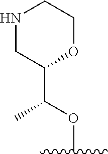 [SMILES: *O[C@H](C)[C@@H]1CNCCO1] | 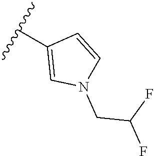 [SMILES: *c1ccn(CC(F)F)c1] | 1H NMR (400 MHz, Methanol- d4) δ 8.87 (dd, 1H), 8.54 (ddd, 1H), 7.51 (app t, 1H), 7.49 (d, 1H), 7.44 (dd, 1H), 6.83 (app t, 1H), 6.73 (dd, 1H), 6.12 (tt, 1H), 5.62-5.52 (m, 1H), 4.38 (td, 2H), 3.97- 3.87 (m, 1H), 3.81-3.75 (m, 1H), 3.72-3.62 (m, 1H), 3.10 (d, 1H), 2.90-2.71 (m, 3H), 1.49 (d, 3H). | 1.72 min, [MH]+ = 389 | 2 | 7-[1-(2,2-difluoroethyl)- 1H-pyrrol-3-yl]-5-[(1R)- 1-[(2S)-morpholin-2- yl]ethoxy]-1,6- naphthyridine |
| 6.31 | 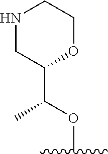 [SMILES: *O[C@H](C)[C@@H]1CNCCO1] | 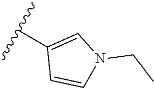 [SMILES: *c1ccn(CC)c1] | 1H NMR (400 MHz, Chloroform- d) δ 8.89 (dd, 1H), 8.41 (ddd, 1H), 7.55 (d, 1H), 7.38 (app t, 1H), 7.29-7.23 (m, 1H), 6.74-6.66 (m, 2H), 5.56 (p, 1H), 4.04-3.92 (m, 3H), 3.77-3.63 (m, 2H), 3.16 (dd, 1H), 2.92 (ddd, 1H), 2.88-2.74 (m, 2H), 1.53-1.45 (m, 6H). | 1.69 min, [MH]+ = 353 | 2 | 7-(1-ethyl-1H-pyrrol-3- yl)-5-[(1R)-1-[(2S)- morpholin-2-yl]ethoxy]- 1,6-naphthyridine |
| 6.32 | 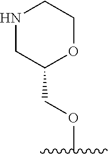 [SMILES: *OC[C@@H]1CNCCO1] | 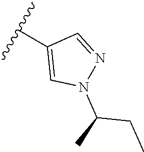 [SMILES: *c1cnn([C@H](C)CC)c1] | 1H NMR (400 MHz, Methanol- d4) δ 8.91 (dd, 1H), 8.59 (ddd, 1H), 8.30 (d, 1H), 8.11 (d, 1H), 7.60 (d, 1H), 7.50 (dd, 1H), 4.61 (d, 2H), 4.39- 4.27 (m, 1H), 4.07-3.97 (m, 1H), 3.92 (ddd, 1H), 3.69 (ddd, 1H), 3.08 (dd, 1H), 2.93-2.74 (m, 3H), 2.04- 1.76 (m, 2H), 1.54 (d, 3H), 0.84 (t, 3H) | 1.70 min, [MH]+ = 368 | 2 | 7-{1-[(2R)-butan-2-yl]- 1H-pyrazol-4-yl}-5- {[(2S)-morpholin-2- yl]methoxy}-1,6- naphthyridine |
| 6.33 | 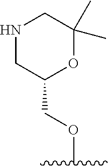 [SMILES: *OC[C@@H]1CNCC(C)(C)O1] | 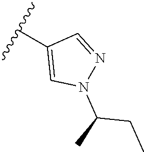 [SMILES: *c1cnn([C@H](C)CC)c1] | 1H NMR (400 MHz, Methanol- d4) δ 8.92 (dd, 1H), 8.59 (ddd, 1H), 8.30 (d, 1H), 8.12 (d, 1H), 7.61 (d, 1H), 7.51 (dd, 1H), 4.59 (dd, 1H), 4.50 (dd, 1H), 4.41-4.30 (m, 1H), 4.30-4.22 (m, 1H), 3.12- 3.04 (m, 1H), 2.75-2.56 (m, 3H), 2.04-1.78 (m, 2H), 1.54 (d, 3H), 1.36 (s, 3H), 1.19 (s, 3H), 0.84 (t, 3H). | 1.83 min, [MH]+ = 396 | 2 | 7-{1-[(2R)-butan-2-yl]- 1H-pyrazol-4-yl}-5- {[(2S)-6,6- dimethylmorpholin-2- yl]methoxy}-1,6- naphthyridine |
| 6.34 | 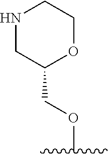 [SMILES: *OC[C@@H]1CNCCO1] | 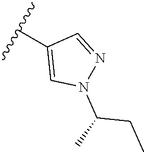 [SMILES: *c1cnn([C@@H](C)CC)c1] | 1H NMR (400 MHz, Methanol- d4) δ 8.91 (dd, 1H), 8.59 (ddd, 1H), 8.30 (d, 1H), 8.11 (d, 1H), 7.59 (d, 1H), 7.49 (dd, 1H), 4.61 (d, 2H), 4.40- 4.27 (m, 1H), 4.09-3.99 (m, 1H), 3.93 (dd, 1H), 3.70 (td, 1H), 3.10 (dd, 1H), 2.93- 2.76 (m, 3H), 2.04-1.78 (m, 2H), 1.53 (d, 3H), 0.84 (t, 3H). | 1.71 min, [MH]+ = 368 | 2 | 7-{1-[(2S)-butan-2-yl]- 1H-pyrazol-4-yl}-5- {[(2S)-morpholin-2- yl]methoxy}-1,6- naphthyridine |
| 6.35 | 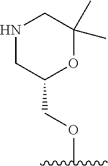 [SMILES: *OC[C@@H]1CNCC(C)(C)O1] | 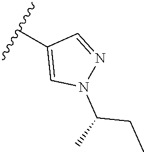 [SMILES: *c1cnn([C@@H](C)CC)c1] | 1H NMR (400 MHz, Methanol- d4) δ 8.92 (dd, 1H), 8.58 (ddd, 1H), 8.30 (d, 1H), 8.11 (d, 1H), 7.60 (d, 1H), 7.50 (dd, 1H), 4.59 (dd, 1H), 4.50 (dd, 1H), 4.40-4.30 (m, 1H), 4.30-4.22 (m, 1H), 3.08 (ddd, 1H), 2.77- 2.55 (m, 3H), 2.05-1.76 (m, 2H), 1.54 (d, 3H), 1.36 (s, 3H), 1.19 (s, 3H), 0.84 (t, 3H). | 1.83 min, [MH]+ = 396 | 2 | 7-{1-[(2S)-butan-2-yl]- 1H-pyrazol-4-yl}-5- {[(2S)-6,6- dimethylmorpholin-2- yl]methoxy}-1,6- naphthyridine |
| 6.36 | 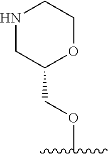 [SMILES: *OC[C@@H]1CNCCO1] | 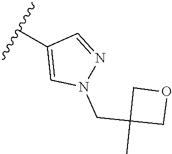 [SMILES: *c1cnn(CC2(C)COC2)c1] | 1H NMR (400 MHz, Methanol- d4) δ 8.87 (dd, 1H), 8.51 (ddd, 1H), 8.24 (d, 1H), 8.06 (d, 1H), 7.52 (d, 1H), 7.44 (dd, 1H), 4.76 (d, 2H), 4.61-4.50 (m, 2H), 4.41 (s, 2H), 4.38 (d, 2H), 4.06-3.95 (m, 1H), 3.95-3.87 (m, 1H), 3.73-3.62 (m, 1H), 3.06 (dd, 1H), 2.92-2.72 (m, 3H), 1.26 (s, 3H). | 1.58 min, [MH]+ = 396 | 2 | 5-{[(2S)-6,6- difluoromorpholin-2- yl]methoxy}-7-(1- methyl-1H-pyrrol-3- yl)quinoline |
| 6.37 | 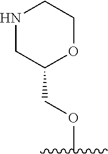 [SMILES: *OC[C@@H]1CNCCO1] | 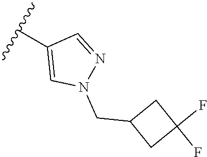 [SMILES: *c1cnn(CC2CC(F)(F)C2)c1] | 1H NMR (400 MHz, Chloroform- d) δ 8.96 (dd, 1H), 8.50 (ddd, 1H), 8.05 (d, 1H), 7.95 (d, 1H), 7.60 (d, 1H), 7.36 (dd, 1H), 4.62 (dd, 1H), 4.56 (dd, 1H), 4.28 (d, 2H), 4.08-4.00 (m, 1H), 3.98 (ddd, 1H), 3.78-3.67 (m, 1H), 3.18-3.10 (m, 1H), 2.97 (ddd, 1H), 2.92- 2.83 (m, 2H), 2.83-2.66 (m, 3H), 2.51-2.36 (m, 2H). | 1.77 min, [MH]+ = 416 | 2 | 7-{1-[(3,3- difluorocyclobutyl)methyl]- 1H-pyrazol-4-yl}-5- {[(2S)-6,6- dimethylmorpholin-2- yl]methoxy}-1,6- naphthyridine |
| 6.38 | 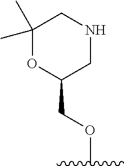 [SMILES: *OC[C@@H]1CNCC(C)(C)O1] | 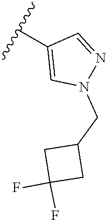 [SMILES: *c1cnn(CC2CC(F)(F)C2)c1] | 1H NMR (600 MHz, Chloroform- d) δ 8.96 (dd, 1H), 8.47 (ddd, 1H), 8.05 (d, 1H), 7.97 (d, 1H), 7.60 (d, 1H), 7.36 (dd, 1H), 4.59 (dd, 1H), 4.44 (dd, 1H), 4.28 (d, 2H), 4.26-4.22 (m, 1H), 3.18 (br s, 1H), 2.80-2.68 (m, 6H), 2.49-2.38 (m, 2H), 1.39 (s, 3H), 1.24 (s, 3H). | 1.88 min, [MH]+ = 444 | 2 | 7-{1-[(3,3- difluorocyclobutyl)methyl]- 1H-pyrazol-4-yl}-5- {[(2S)-6,6- dimethylmorpholin-2- yl]methoxy}-1,6- naphthyridine |
| 6.39 | 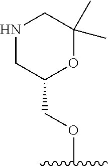 [SMILES: *OC[C@@H]1CNCC(C)(C)O1] | 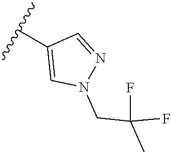 [SMILES: *c1cnn(CC(C)(F)F)c1] | 1H NMR (600 MHz, Chloroform- d) δ 8.96 (dd, 1H), 8.48 (ddd, 1H), 8.09 (s, 1H), 8.08 (s, 1H), 7.62 (d, 1H), 7.37 (dd, 1H), 4.59 (dd, 1H), 4.52 (t, 2H), 4.44 (dd, 1H), 4.24 (dddd, 1H), 3.16 (dd, 1H), 2.75-2.65 (m, 3H), 1.63 (t, 3H), 1.38 (s, 3H), 1.23 (s, 3H). | 1.77 min, [MH]+ = 418 | 2 | 7-[1-(2,2- difluoropropyl)-1H- pyrazol-4-yl]-5-{[(2S)- 6,6-dimethylmorpholin- 2-yl]methoxy}-1,6- naphthyridine |
| 6.40 | 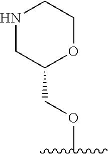 [SMILES: *OC[C@@H]1CNCCO1] | 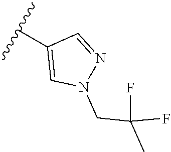 [SMILES: *c1cnn(CC(C)(F)F)c1] | 1H NMR (600 MHz, Chloroform- d) δ 8.98-8.94 (m, 1H), 8.53-8.48 (m, 1H), 8.09 (s, 1H), 8.06 (s, 1H), 7.62 (s, 1H), 7.37 (dd, 1H), 7.26 (d, 1H), 4.61 (dd, 1H), 4.57 (dd, 1H), 4.52 (t, 2H), 4.05-3.99 (m, 1H), 3.99- 3.94 (m, 1H), 3.72 (td, 1H), 3.13 (dd, 1H), 2.97 (td, 1H), 2.90-2.83 (m, 2H), 1.63 (t, 3H). | 1.66 min, [MH]+ = 390 | 2 | 7-[1-(2,2- difluoropropyl)-1H- pyrazol-4-yl]-5-{[(2S)- morpholin-2- yl]methoxy}-1,6- naphthyridine |
| 6.41 | 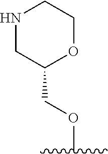 [SMILES: *OC[C@@H]1CNCCO1] | 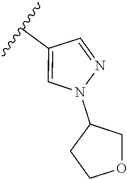 [SMILES: *c1cnn(C2CCOC2)c1] | 1H NMR (400 MHz, Methanol- d4) δ 8.92 (dd, 1H), 8.59 (ddd, 1H), 8.32 (d, 1H), 8.12 (d, 1H), 7.60 (d, 1H), 7.50 (dd, 1H), 5.11 (ddt, 1H), 4.61 (s, 1H), 4.60 (s, 1H), 4.22-4,13 (m, 1H), 4.11- 4.07 (m, 2H), 4.03 (dddd, 1H), 3.98-3.90 (m, 2H), 3.69 (ddd, 1H), 3.08 (dd, 1H), 2.93-2.74 (m, 3H), 2.54 (dtd, 1H), 2.39 (dddd, 1H). | 1.54 min, [MH]+ = 382 | 2 | 5-{[(2S)-morpholin-2- yl]methoxy}-7-{1-[(3S)- oxolan-3-yl]-1H- pyrazol-4-yl}-1,6- naphthyridine |
| 6.42 | 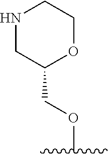 [SMILES: *OC[C@@H]1CNCCO1] | 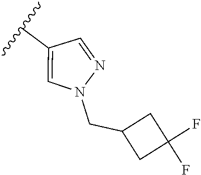 [SMILES: *c1cnn(CC2CC(F)(F)C2)c1] | 1H NMR (400 MHz, Methanol- d4) δ 8.86 (dd, 1H), 8.55 (ddd, 1H), 7.49 (dd, 1H), 7.48 (d, 1H), 7.43 (dd, 1H), 6.80 (dd, 1H), 6.69 (dd, 1H), 4.67-4.54 (m, 2H), 4.12-4.06 (m, 2H), 4.06- 3.99 (m, 1H), 3.96-3.91 (m, 1H), 3.70 (ddd, 1H), 3.10 (dd, 1H), 2.94-2.75 (m, 3H), 2.73-2.57 (m, 3H), 2.47-2.32 (m, 2H). | 1.82 min, [MH]+ = 415 | 2 | 7-{1-[(3,3- difluorocyclobutyl)methyl]- 1H-pyrrol-3-yl}-5- {[(2S)-morpholin-2- yl]methoxy}-1,6- naphthyridine |
| 6.43 | 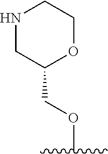 [SMILES: *OC[C@@H]1CNCCO1] | 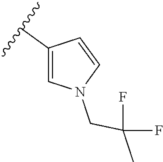 [SMILES: *c1ccn(CC(C)(F)F)c1] | 1H NMR (400 MHz, Methanol- d4) δ 8.88 (ddd, 1H), 8.57 (ddd, 1H), 7.55-7.48 (m, 2H), 7.45 (ddd, 1H), 6.82 (app t, 1H), 6.73 (ddd, 1H), 4.65-4.56 (m, 2H), 4.37 (t, 2H), 4.07-3.99 (m, 1H), 3.97-3.89 (m, 1H), 3.70 (td, 1H), 3.09 (dd, 1H), 2.93-2.75 (m, 3H), 1.59 (t, 3H). | 1.72 min, [MH]+ = 389 | 2 | 7-[1-(2,2- difluoropropyl)-1H- pyrrol-3-yl]-5-{[(2S)- morpholin-2- yl]methoxy}-1,6- naphthyridine |
| 6.44 | 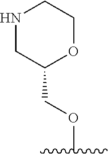 [SMILES: *OC[C@@H]1CNCCO1] | 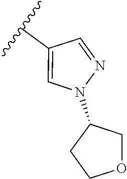 [SMILES: *c1cnn([C@H]2CCOC2)c1] | 1H NMR (400 MHz, Methanol- d4) δ 8.95 (dd, 1H), 8.61 (ddd, 1H), 8.34 (d, 1H), 8.14 (d, 1H), 7.64 (d, 1H), 7.52 (dd, 1H), 5.16-5.07 (m, 1H), 4.79-4.68 (m, 2H), 4.30-4.22 (m, 1H), 4.22- 4.05 (m, 4H), 3.99-3.81 (m, 2H), 3.49 (dd, 1H), 3.28- 3.15 (m, 3H), 2.60-2.49 (m, 1H), 2.43-2.35 (m, 1H). | 1.54 min, [MH]+ = 382 | 2 | 5-{[(2S)-morpholin-2- yl]methoxy}-7-{1-[(3S)- oxolan-3-yl]-1H- pyrazol-4-yl}-1,6- naphthyridine |
| 6.45 | 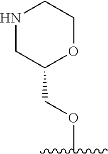 [SMILES: *OC[C@@H]1CNCCO1] | 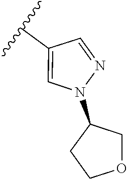 [SMILES: *c1cnn([C@@H]2CCOC2)c1] | 1H NMR (400 MHz, Methanol- d4) δ 8.96 (dd, 1H), 8.62 (ddd, 1H), 8.34 (d, 1H), 8.14 (d, 1H), 7.65 (d, 1H), 7.53 (dd, 1H), 5.15-5.08 (m, 1H), 4.78-4.73 (m, 2H), 4.32-4.25 (m, 1H), 4.22- 4.14 (m, 2H), 4.13-4.05 (m, 2H), 3.99-3.87 (m, 2H), 3.56 (dd, 1H), 3.36-3.20 (m, 3H), 2.61-2.49 (m, 1H), 2.44-2.34 (m, 1H). | 1.53 min, [MH]+ = 382 | 2 | 5-{[(2S)-morpholin-2- yl]methoxy}-7-{1-[(3R)- oxolan-3-yl]-1H- pyrazol-4-yl}-1,6- naphthyridine |

TABLE 7
Analytical data for quinolines synthesised by general method D:

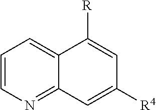

| | | | | LC MS RT, | LC MS | |
|---|---|---|---|---|---|---|
| No | R | R⁴ | 1H NMR | m/z | Method | Name |
| 7.1 | 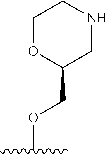 [SMILES: *OC[C@@H]1CNCCO1] | 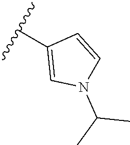 [SMILES: *c1ccn(C(C)C)c1] | 1H NMR (400 MHz, Methanol- d4) δ 8.71 (dd, 1H), 8.56 (ddd, 1H), 7.67 (app t, 1H), 7.41 (app t, 1H), 7.35 (dd, 1H), 7.21 (d, 1H), 6.86 (dd, 1H), 6.58 (dd, 1H), 4.33 (hept, 1H), 4.29-4.19 (m, 2H), 4.03- 3.98 (m, 1H), 3.98-3.91 (m, 1H), 3.77-3.68 (m, 1H), 3.10 (dd, 1H), 2.94-2.80 (m, 3H), 1.50 (d, 6H). | 1.50 min, [MH]+ = 352 | 2 | 5-{[(2S)-morpholin-2- yl]methoxy}-7-[1-(propan- 2-yl)-1H-pyrrol-3- yl]quinoline |
| 7.2 | 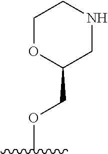 [SMILES: *OC[C@@H]1CNCCO1] | 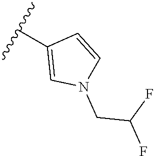 [SMILES: *c1ccn(CC(F)F)c1] | 1H NMR (400 MHz, Methanol- d4) δ 8.73 (dd, 1H), 8.57 (ddd, 1H), 7.68 (app t, 1H), 7.40-7.35 (m, 2H), 7.19 (d, 1H), 6.85 (app t, 1H), 6.65 (dd, 1H), 6.12 (tt, 1H), 4.37 (td, 2H), 4.26 (dd, 1H), 4.21 (dd, 1H), 4.01 (dddd, 1H), 3.97-3.91 (m, 1H), 3.72 (ddd, 1H), 3.10 (dd, 1H), 2.96- 2.79 (m, 3H). | 1.40 min, [MH]+ = 374 | 2 | 7-[1-(2,2-difluoroethyl)- 1H-pyrrol-3-yl]-5-{[(2S)- morpholin-2- yl]methoxy}quinoline |
| 7.3 | 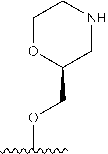 [SMILES: *OC[C@@H]1CNCCO1] | 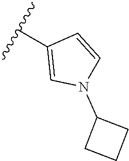 [SMILES: *c1ccn(C2CCC2)c1] | 1H NMR (600 MHz, Methanol- d4) δ 8.73 (dd, 1H), 8.61- 8.54 (m, 1H), 7.68 (s, 1H), 7.45 (app t, 1H), 7.37 (dd, 1H), 7.23 (s, 1H), 6.89 (app t, 1H), 6.59 (app t, 1H), 4.63 (p, 1H), 4.28 (dd, 1H), 4.24 (dd, 1H), 4.08-3.99 (m, 1H), 3.99-3.93 (m, 1H), 3.74 (td, 1H), 3.16-3.09 (m, 1H), 2.95-2.83 (m, 3H), 2.53- 2.39 (m, 4H), 1.97-1.80 (m, 2H) | 1.55 min, [MH]+ = 364 | 2 | 7-(1-cyclobutyl-1H-pyrrol- 3-yl)-5-{[(2S)-morpholin- 2-yl]methoxy}quinoline |
| 7.4 | 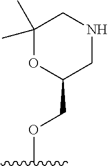 [SMILES: *OC[C@@H]1CNCC(C)(C)O1] | 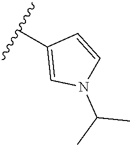 [SMILES: *c1ccn(C(C)C)c1] | 1H NMR (600 MHz, DMSO- d6) δ 8.79 (dd, 1H), 8.37 (ddd, 1H), 7.67-7.62 (m, 2H), 7.35 (dd, 1H), 7.25 (d, 1H), 6.92 (t, 1H), 6.60 (dd, 1H), 4.31 (hept, 1H), 4.17 (dd, 1H), 4.14-4.04 (m, 2H), 3.05 (dd, 1H), 2.61 (d, 1H), 2.49- 2.44 (m, 2H), 1.44 (d, 6H), 1.31 (s, 3H), 1.10 (s, 3H). | 1.65 min, [MH]+ = 380 | 2 | 5-{[(2S)-6,6- dimethylmorpholin-2- yl]methoxy}-7-[1-(propan- 2-yl)-1H-pyrrol-3- yl]quinoline |
| 7.5 | 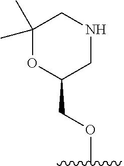 [SMILES: *OC[C@@H]1CNCC(C)(C)O1] | 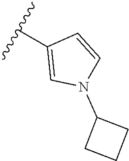 [SMILES: *c1ccn(C2CCC2)c1] | 1H NMR (600 MHz, DMSO- d6) δ 8.80 (dd, 1H), 8.38 (dd, 1H), 7.72 (app t, 1H), 7.66 (s, 1H), 7.36 (dd, 1H), 7.27 (d, 1H), 6.95 (app t, 1H), 6.62 (dd, 1H), 4.61 (p, 1H), 4.18 (dd, 1H), 4.14-4.02 (m, 2H), 3.06 (dd, 1H), 2.61 (d, 1H), 2.48-2.44 (m, 2H), 2.43-2.36 (m, 4H), 1.85-1.71 (m, 2H), 1.31 (s, 3H), 1.11 (s, 3H). | 1.71 min, [MH]+ = 392 | 2 | 7-(1-cyclobutyl-1H-pyrrol- 3-yl)-5-{[(2S)-6,6- dimethylmorpholin-2- yl]methoxy}quinoline |
| 7.6 | 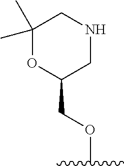 [SMILES: *OC[C@@H]1CNCC(C)(C)O1] | 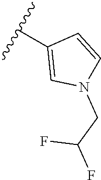 [SMILES: *c1ccn(CC(F)F)c1] | 1H NMR (600 MHz, DMSO- d6) δ 8.81 (dd, 1H), 8.40 (dd, 1H), 7.67 (app t, 1H), 7.57 (app t, 1H), 7.38 (dd, 1H), 7.23 (d, 1H), 6.91 (app t, 1H), 6.71 (dd, 1H), 6.35 (tt, 1H), 4.43 (td, 2H), 4.21-4.14 (m, 1H), 4.13-4.04 (m, 2H), 3.05 (dd, 1H), 2.62 (d, 1H), 2.49-2.45 (m, 2H), 1.31 (s, 3H), 1.10 (s, 3H). | 1.55 min, [MH]+ = 402 | 2 | 7-[1-(2,2-difluoroethyl)- 1H-pyrrol-3-yl]-5-{[(2S)- 6,6-dimethylmorpholin-2- yl]methoxy}quinoline |
| 7.7 | 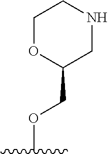 [SMILES: *OC[C@@H]1CNCCO1] | 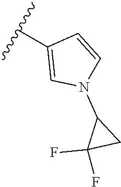 [SMILES: *c1ccn(C2CC2(F)F)c1] | 1H NMR (400 MHz, Methanol- d4) δ 8.73 (dd, 1H), 8.56 (ddd, 1H), 7.67 (dd, 1H), 7.40 (app t, 1H), 7.37 (dd, 1H), 7.20 (d, 1H), 6.86 (app t, 1H), 6.64 (dd, 1H), 4.33-4.17 (m, 2H), 4.17-4.06 (m, 1H), 4.01 (dddd, 1H), 3.98-3.90 (m, 1H), 3.72 (ddd, 1H), 3.11 (dd, 1H), 2.96-2.77 (m, 3H), 2.21-2.05 (m, 2H). | 1.51 min, [MH]+ = 386 | 2 | 7-[1-(2,2- difluorocyclopropyl)-1H- pyrrol-3-yl]-5-{[(2S)- morpholin-2- yl]methoxy}quinoline |
| 7.8 | 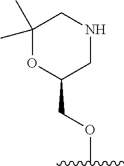 [SMILES: *OC[C@@H]1CNCC(C)(C)O1] | 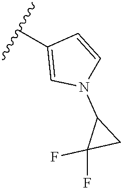 [SMILES: *c1ccn(C2CC2(F)F)c1] | 1H NMR (400 MHz, Methanol- d4) δ 8.72 (dd, 1H), 8.54 (ddd, 1H), 7.67 (app t, 1H), 7.39 (app t, 1H), 7.37 (dd, 1H), 7.20 (d, 1H), 6.86 (app t, 1H), 6.64 (dd, 1H), 4.31- 4.02 (m, 4H), 3.11 (dd, 1H), 2.80-2.56 (m, 3H), 2.21- 2.05 (m, 2H), 1.38 (s, 3H), 1.20 (s, 3H). | 1.64 min, [MH]+ = 414 | 2 | 7-[1-(2,2- difluorocyclopropyl)-1H- pyrrol-3-yl]-5-{[(2S)-6,6- dimethylmorpholin-2- yl]methoxy}quinoline |
| 7.9 | 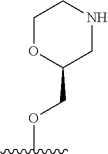 [SMILES: *OC[C@@H]1CNCCO1] | 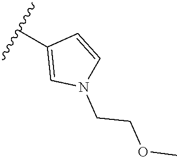 [SMILES: *c1ccn(CCOC)c1] | 1H NMR (400 MHz, Methanol- d4) δ 8.74 (dd, 1H), 8.57 (ddd, 1H), 7.69 (dd, 1H), 7.37 (dd, 1H), 7.34 (app t, 1H), 7.22 (d, 1H), 6.83 (dd, 1H), 6.59 (dd, 1H), 4.42-4.30 (m, 2H), 4.28-4.20 (m, 1H), 4.19-4.14 (m, 1H), 4.12 (t, 2H), 3.91 (ddd, 1H), 3.70 (dd, 2H), 3.52 (dd, 1H), 3.35 (s, 3H), 3.29-3.21 (m, 3H). | 1.39 min, [MH]+ = 368 | 2 | 7-[1-(2-methoxyethyl)-1H- pyrrol-3-yl]-5-{[(2S)- morpholin-2- yl]methoxy}quinoline |
| 7.10 | 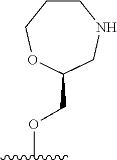 [SMILES: *OC[C@@H]1CNCCCO1] | 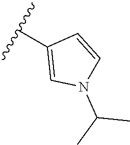 [SMILES: *c1ccn(C(C)C)c1] | 1H NMR (600 MHz, Methanol- d4) δ 8.74 (dd, 1H), 8.59 (ddd, 1H), 7.69 (app t, 1H), 7.43 (app t, 1H), 7.38 (dd, 1H), 7.25 (d, 1H), 6.88 (app t, 1H), 6.60 (dd, 1H), 4.36 (hept, 1H), 4.28 (dd, 1H), 4.23 (dd, 1H), 4.20-4.15 (m, 1H), 4.09 (ddd, 1H), 3.86 (ddd, 1H), 3.29 (dd, 1H), 3.06 (ddd, 1H), 3.02-2.94 (m, 3H), 2.03-1.90 (m, 2H), 1.53 (d, 6H). | 1.53 min, [MH]+ = 366 | 2 | 5-{[(2S)-1,4-oxazepan-2- yl]methoxy}-7-[1-(propan- 2-yl)-1H-pyrrol-3- yl]quinoline |
| 7.11 | 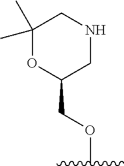 [SMILES: *OC[C@@H]1CNCC(C)(C)O1] | 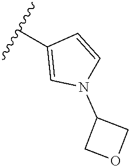 [SMILES: *c1ccn(C2COC2)c1] | 1H NMR (400 MHz, Methanol- d4) δ 8.73 (dd, 1H), 8.57 (ddd, 1H), 7.70 (dd, 1H), 7.64 (app t, 1H), 7.38 (dd, 1H), 7.25 (d, 1H), 7.06 (dd, 1H), 6.68 (dd, 1H), 5.37 (tt, 1H), 5.14-5.06 (m, 2H), 4.98- 4.88 (m, 2H), 4.32-4.10 (m, 3H), 3.10 (ddd, 1H), 2.76- 2.57 (m, 3H), 1.38 (s, 3H), 1.20 (s, 3H). | 1.46 min, [MH]+ = 394 | 2 | 5-{[(2S)-6,6- dimethylmorpholin-2- yl]methoxy}-7-[1-(oxetan- 3-yl)-1H-pyrrol-3- yl]quinoline |
| 7.12 | 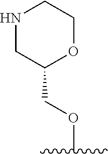 [SMILES: *OC[C@@H]1CNCCO1] | 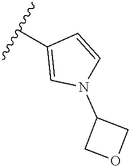 [SMILES: *c1ccn(C2COC2)c1] | 1H NMR (400 MHz, Methanol- d4) δ 8.73 (dd, 1H), 8.58 (ddd, 1H), 7.70 (dd, 1H), 7.65 (app t, 1H), 7.38 (dd, 1H), 7.25 (d, 1H), 7.07 (dd, 1H), 6.68 (dd, 1H), 5.37 (tt, 1H), 5.14-5.06 (m, 2H), 4.98-4.90 (m, 2H), 4.28 (dd, 1H), 4.28 (dd, 1H), 4.01 (dddd, 1H), 3.97-3.90 (m, 1H), 3.72 (ddd, 1H), 3.09 (dd, 1H), 2.94-2.78 (m, 3H). | 1.36 min, [MH]+ = 366 | 2 | 5-{[(2S)-morpholin-2- yl]methoxy}-7-[1-(oxetan- 3-yl)-1H-pyrrol-3- yl]quinoline |
| 7.13 | 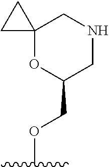 [SMILES: *OC[C@@H]1CNCC2(CC2)O1] | 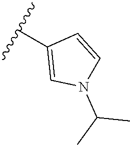 [SMILES: *c1ccn(C(C)C)c1] | 1H NMR (400 MHz, Methanol- d4) δ 8.72 (dd, 1H), 8.57- 8.50 (m, 1H), 7.67 (app t, 1H), 7.40 (app t, 1H), 7.36 (dd, 1H), 7.21 (d, 1H), 6.86 (app t, 1H), 6.57 (dd, 1H), 4.33 (hept, 1H), 4.28-4.13 (m, 3H), 3.37-3.28 (m, 1H), 3.17 (dd, 1H), 2.91 (dd, 1H), 2.37 (d, 1H), 1.50 (d, 6H), 0.92- 0.85 (m, 1H), 0.82-0.76 (m, 1H), 0.69-0.57 (m, 2H). | 1.69 min, [MH]+ = 378 | 2 | 5-{[(5S)-4-oxa-7- azaspiro[2.5]octan-5- yl]methoxy}-7-[1-(propan- 2-yl)-1H-pyrrol-3- yl]quinoline |
| 7.14 | 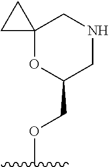 [SMILES: *OC[C@@H]1CNCC2(CC2)O1] | 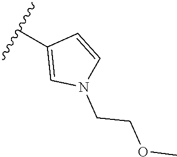 [SMILES: *c1ccn(CCOC)c1] | 1H NMR (400 MHz, Methanol- d4) δ 8.72 (dd, 1H), 8.53 (ddd, 1H), 7.67 (app t, 1H), 7.36 (dd, 1H), 7.33 (app t, 1H), 7.19 (d, 1H), 6.82 (dd, 1H), 6.57 (dd, 1H), 4.27- 4.14 (m, 3H), 4.11 (t, 2H), 3.70 (t, 2H), 3.39-3.32 (m, 4H), 3.17 (dd, 1H), 2.91 (dd, 1H), 2.37 (d, 1H), 0.95- 0.83 (m, 1H), 0.83-0.72 (m, 1H), 0.70-0.57 (m, 2H). | 1.52 min, [MH]+ = 394 | 2 | 7-[1-(2-methoxyethyl)-1H- pyrrol-3-yl]-5-{[(5S)-4- oxa-7-azaspiro[2.5]octan- 5-yl]methoxy }quinoline |
| 7.15 | 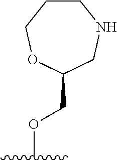 [SMILES: *OC[C@@H]1CNCCCO1] | 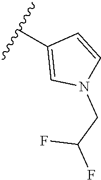 [SMILES: *c1ccn(CC(F)F)c1] | 1H NMR (400 MHz, Methanol- d4) δ 8.78 (dd, 1H), 8.60 (ddd, 1H), 8.28 (s, 1H), 8.10 (d, 1H), 7.73 (t, 1H), 7.43 (dd, 1H), 7.22 (d, 1H), 6.25 (tt, 1H), 4.63 (td, 2H), 4.31- 4.14 (m, 3H), 4.12-4.00 (m, 1H), 3.83 (ddd, 1H), 3.37- 3.32 (m, 1H), 3.16-2.96 (m, 3H), 2.07-1.90 (m, 2H). | 1.38 min, [MH]+ = 389 | 2 | 7-[1-(2,2-difluoroethyl)- 1H-pyrazol-4-yl]-5-{[(2S)- 1,4-oxazepan-2- yl]methoxy}quinoline |
| 7.16 | 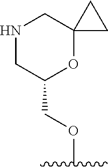 [SMILES: *OC[C@@H]1CNCC2(CC2)O1] | 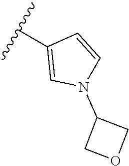 [SMILES: *c1ccn(C2COC2)c1] | 1H NMR (400 MHz, Methanol- d4) δ 8.74 (dd, 1H), 8.58 8.54 (m, 1H), 7.71 (s, 1H), 7.65 (app t, 1H), 7.39 (ddd, 1H), 7.25 (d, 1H), 7.07 (dd, 1H), 6.69 (dd, 1H), 5.44- 5.33 (m, 1H), 5.11 (t, 2H), 4.95 (t, 1H), 4.30-4.21 (m, 2H), 4.20-4.13 (m, 1H), 3.38- 3.27 (m, 1H), 3.16 (dd, 1H), 2.90 (dd, 1H), 2.35 (dd, 1H), 0.93-0.82 (m, 1H), 0.84-0.72 (m, 1H), 0.69-0.58 (m, 2H). | 1.44 min, [MH]+ = 392 | 2 | 5-{[(5S)-4-oxa-7- azaspiro[2.5]octan-5- yl]methoxy}-7-[1-(oxetan- 3-yl)-1H-pyrrol-3- yl]quinoline |
| 7.17 | 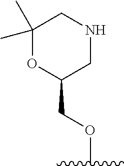 [SMILES: *OC[C@@H]1CNCC(C)(C)O1] | 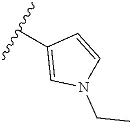 [SMILES: *c1ccn(CC)c1] | 1H NMR (400 MHz, Methanol- d4) δ 8.71 (dd, 1H), 8.55 (ddd, 1H), 7.68-7.63 (m, 1H), 7.39-7.30 (m, 2H), 7.21 (d, 1H), 6.79 (dd, 1H), 6.56 (dd, 1H), 4.29-4.09 (m, 3H), 4.00 (q, 2H), 3.09 (ddd, 1H), 2.78-2.56 (m, 3H), 1.45 (t, 3H), 1.38 (s, 3H), 1.19 (s, 3H). | 1.52 min, [MH]+ = 366 | 2 | 5-{[(2S)-6,6- dimethylmorpholin-2- yl]methoxy}-7-(1-ethyl- 1H-pyrrol-3-yl)quinoline |
| 7.18 | 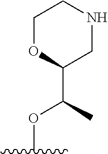 [SMILES: *O[C@H](C)[C@@H]1CNCCO1] | 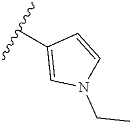 [SMILES: *c1ccn(CC)c1] | 1H NMR (400 MHz, Methanol- d4) δ 8.70 (dd, 1H), 8.52 (ddd, 1H), 7.66 (app t, 1H), 7.36-7.29 (m, 2H), 7.25 (d, 1H), 6.78 (dd, 1H), 6.56 (dd, 1H), 4.74-4.64 (m, 1H), 3.98 (q, 2H), 3.90 (ddd, 1H), 3.74-3.59 (m, 2H), 3.13 (dd, 1H), 2.86-2.69 (m, 3H), 1.46-1.41 (m, 6H). | 1.52 min, [MH]+ = 352 | 2 | 7-(1-ethyl-1H-pyrrol-3-yl)- 5-[(1R)-1-[(2S)- morpholin-2- yl]ethoxy]quinoline |
| 7.19 | 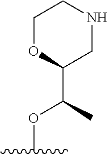 [SMILES: *O[C@H](C)[C@@H]1CNCCO1] | 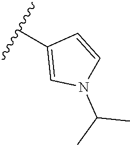 [SMILES: *c1ccn(C(C)C)c1] | 1H NMR (400 MHz, Methanol- d4) δ 8.71 (dd, 1H), 8.54 (ddd, 1H), 7.66 (dd, 1H), 7.39 (app t, 1H), 7.34 (dd, 1H), 7.29-7.24 (m, 1H), 6.85 (dd, 1H), 6.56 (dd, 1H), 4.76- 4.67 (m, 1H), 4.33 (hept, 1H), 3.96-3.88 (m, 1H), 3.72 (ddd, 1H), 3.66 (ddd, 1H), 3.15 (dd, 1H), 2.83-2.74 (m, 3H), 1.50 (d, 6H), 1.44 (d, 3H). | 1.58 min, [MH]+ = 366 | 2 | 5-[(1R)-1-[(2S)- morpholin-2-yl]ethoxy]-7- [1-(propan-2-yl)-1H- pyrrol-3-yl]quinoline |
| 7.20 | 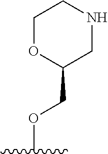 [SMILES: *OC[C@@H]1CNCCO1] | 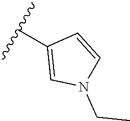 [SMILES: *c1ccn(CC)c1] | 1H NMR (400 MHz, Methanol- d4) δ 8.71 (dd, 1H), 8.55 (ddd, 1H), 7.66 (dd, 1H), 7.38-7.30 (m, 2H), 7.19 (d, 1H), 6.79 (dd, 1H), 6.56 (dd, 1H), 4.25 (dd, 1H), 4.20 (dd, 1H), 4.05-3.90 (m, 4H), 3.71 (ddd, 1H), 3.09 (dd, 1H), 2.94-2.77 (m, 3H), 1.45 (t, 3H). | 1.42 min, [MH]+ = 338 | 2 | 7-(1-ethyl-1H-pyrrol-3-yl)- 5-{[(2S)-morpholin-2- yl]methoxy}quinoline |
| 7.21 | 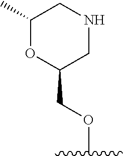 [SMILES: *OC[C@@H]1CNC[C@@H](C)O1] | 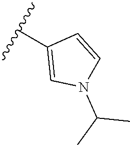 [SMILES: *c1ccn(C(C)C)c1] | 1H NMR (400 MHz, Methanol- d4) δ 8.76 (dd, 1H), 8.62 (d, 1H), 7.71 (d, 1H), 7.46- 7.37 (m, 2H), 7.31 (s, 1H), 6.88 (app t, 1H), 6.60 (app t, 1H), 4.63-4.51 (m, 2H), 4.47 (dd, 1H), 4.39-4.28 (m, 2H), 3.53-3.41 (m, 2H), 3.36 (dd, 1H), 3.12-3.04 (m, 1H), 1.51 (d, 6H), 1.38 (d, 3H) | 1.62 min, [MH]+ = 423 | 2 | 5-{[(2S,6R)-6- methylmorpholin-2- yl]methoxy}-7-[1-(propan- 2-yl)-1H-pyrrol-3- yl]quinoline |

TABLE 8
Analytical data for quinoxalines synthesised by general method D:

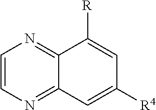

| | | | | LC MS RT, |
|---|---|---|---|---|
| No | R | R⁴ | 1H NMR | m/z |
| 8.1 | 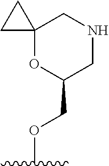 [SMILES: *OC[C@@H]1CNCC2(CC2)O1] | 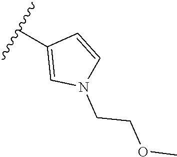 [SMILES: *c1ccn(CCOC)c1] | 1H NMR (400 MHz, Methanol-d4) δ 8.75 (d, 1H), 8.64 (d, 1H), 7.65 (d, 1H), 7.42 (d, 1H), 7.36 (app t, 1H), 6.82 (dd, 1H), 6.57 (dd, 1H), 4.32 − 4.23 (m, 1H), 4.23 − 4.15 (m, 2H), 4.11 (t, 2H), 3.69 (dd, 2H), 3.34 (s, 3H), 3.32 − 3.28 (m, 1H-overlapping with methanol peak), 3.24 − 3.18 (m, 1H), 2.87 (dd, 1H), 2.38 − 2.30 (m, 1H), 0.89 − 0.82 (m, 2H), 0.63 − 0.56 (m, 2H). | 1.75 min, [MH]+ = 395 |
| 8.2 | 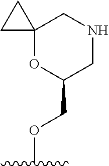 [SMILES: *OC[C@@H]1CNCC2(CC2)O1] | 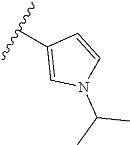 [SMILES: *c1ccn(C(C)C)c1] | 1H NMR (400 MHz, Methanol-d4) δ 8.80 (d, 1H), 8.67 (d, 1H), 7.71 (d, 1H), 7.48 (d, 1H), 7.46 (app t, 1H), 6.87 (dd, 1H), 6.59 (dd, 1H), 4.47 − 4.28 (m, 4H), 3.68 (t, 2H), 3.49 (dd, 1H), 2.94 (dd, 1H), 1.50 (d, 6H), 1.04 (d, 2H), 0.88 − 0.75 (m, 2H). | 1.91 min, [MH]+ = 379 |
| 8.3 | 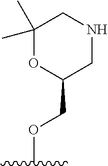 [SMILES: *OC[C@@H]1CNCC(C)(C)O1] | 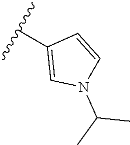 [SMILES: *c1ccn(C(C)C)c1] | 1H NMR (400 MHz, Methanol-d4) δ 8.78 (d, 1H), 8.66 (d, 1H), 7.69 (d, 1H), 7.52 (d, 1H), 7.46 (app t, 1H), 6.87 (dd, 1H), 6.59 (dd, 1H), 4.42 − 4.24 (m, 3H), 4.23 − 4.14 (m, 1H), 3.21 − 3.12 (m, 1H), 2.74 − 2.57 (m, 3H), 1.50 (d, 6H), 1.37 (s, 3H), 1.18 (s, 3H). | 1.91 min, [MH]+ = 381 |
| 8.4 | 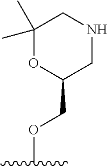 [SMILES: *OC[C@@H]1CNCC(C)(C)O1] | 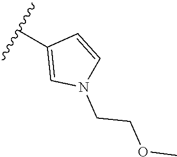 [SMILES: *c1ccn(CCOC)c1] | 1H NMR (400 MHz, Methanol-d4) δ 8.77 (d, 1H), 8.66 (dd, 1H), 7.68 (dd, 1H), 7.49 (d, 1H), 7.38 (app t, 1H), 6.83 (dd, 1H), 6.62 − 6.56 (m, 1H), 4.33 − 4.22 (m, 2H), 4.20 − 4.14 (m, 1H), 4.12 (t, 2H), 3.73 − 3.66 (m, 2H), 3.35 (s, 3H), 3.16 (ddd, 1H), 2.74 − 2.56 (m, 3H), 1.37 (s, 3H), 1.18 (s, 3H). | 1.78 min, [MH]+ = 397 |
| 8.5 | 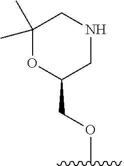 [SMILES: *OC[C@@H]1CNCC(C)(C)O1] | 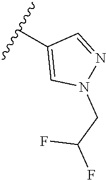 [SMILES: *c1cnn(CC(F)F)c1] | 1H NMR (400 MHz, Methanol-d4) δ 8.77 (dd, 1H), 8.66 (dd, 1H), 7.68 (app t, 1H), 7.46 (app t, 1H), 7.39 (app t, 1H), 6.85 (app t, 1H), 6.65 (ddd, 1H), 6.12 (tt, 1H), 4.37 (td, 2H), 4.31 − 4.21 (m, 2H), 4.20 − 4.11 (m, 1H), 3.16 (ddd, 1H), 2.74 − 2.56 (m, 3H), 1.37 (s, 3H), 1.17 (s, 3H). | 1.82 min, [MH]+ = 403 |
| 8.6 | 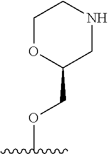 [SMILES: *OC[C@@H]1CNCCO1] | 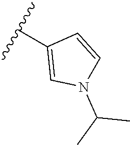 [SMILES: *c1ccn(C(C)C)c1] | 1H NMR (400 MHz, Methanol-d4) δ 8.77 (d, 1H), 8.66 (d, 1H), 7.69 (d, 1H), 7.49 (d, 1H), 7.47 (app t, 1H), 6.87 (dd, 1H), 6.60 (dd, 1H), 4.40 − 4.29 (m, 2H), 4.24 (dd, 1H), 4.12 − 4.01 (m, 1H), 3.95 − 3.87 (m, 1H), 3.71 (td, 1H), 3.17 − 3.09 (m, 1H), 2.93 − 2.76 (m, 3H), 1.50 (d, 6H). | 1.84 min, [MH]+ = 353 |
| 8.7 | 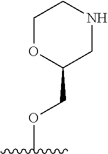 [SMILES: *OC[C@@H]1CNCCO1] | 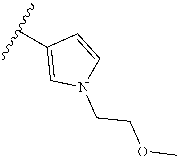 [SMILES: *c1ccn(CCOC)c1] | 1H NMR (400 MHz, Methanol-d4) δ 8.78 (d, 1H), 8.66 (d, 1H), 7.69 (d, 1H), 7.47 (d, 1H), 7.39 (app t, 1H), 6.83 (dd, 1H), 6.60 (dd, 1H), 4.31 (dd, 1H), 4.23 (dd, 1H), 4.12 (t, 2H), 4.06 (dddd, 1H), 3.95 − 3.87 (m, 1H), 3.76 − 3.65 (m, 3H), 3.35 (s, 3H), 3.13 (dd, 1H), 2.93 − 2.75 (m, 3H). | 1.68 min, [MH]+ = 369 |
| 8.8 | 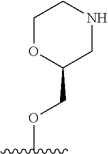 [SMILES: *OC[C@@H]1CNCCO1] | 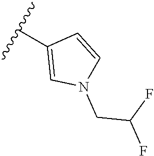 [SMILES: *c1ccn(CC(F)F)c1] | 1H NMR (400 MHz, Methanol-d4) δ 8.79 (d, 1H), 8.68 (d, 1H), 7.71 (d, 1H), 7.48 (d, 1H), 7.42 (app t, 1H), 6.86 (app t, 1H), 6.67 (dd, 1H), 6.12 (tt, 1H), 4.45 − 4.20 (m, 4H), 4.12 − 4.01 (m, 1H), 3.96 − 3.88 (m, 1H), 3.71 (td, 1H), 3.14 (dd, 1H), 2,94 − 2.76 (m, 3H). | 1.71 min, [MH]+ = 375 |
| 8.9 | 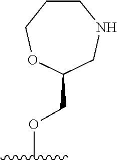 [SMILES: *OC[C@@H]1CNCCCO1] | 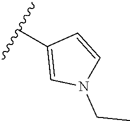 [SMILES: *c1ccn(CC)c1] | 1H NMR (400 MHz, Methanol-d4) δ 8.78 (d, 1H), 8.67 (d, 1H), 7.69 (d, 1H), 7.48 (d, 1H), 7.44 − 7.37 (m, 1H), 6.82 (dd, 1H), 6.60 (dd, 1H), 4.33 (dd, 1H), 4.29 − 4.14 (m, 2H), 4.08 − 3.96 (m, 3H), 3.83 (ddd, 1H), 3.35 − 3.31 (m, 1H), 3.09 − 2.94 (m, 3H), 2.02 − 1.82 (m, 2H), 1.46 (t, 3H). | 1.78 min, [MH]+ = 353 |
| 8.10 | 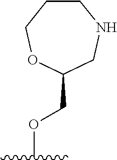 [SMILES: *OC[C@@H]1CNCCCO1] | 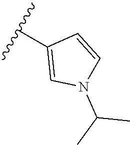 [SMILES: *c1ccn(C(C)C)c1] | 1H NMR (400 MHz, Methanol-d4) δ 8.77 (d, 1H), 8.67 (d, 1H), 7.69 (d, 1H), 7.52 − 7.44 (m, 2H), 6.87 (dd, 1H), 6.60 (dd, 1H), 4.41 − 4.29 (m, 2H), 4.26 (dd, 1H), 4.23 − 4.16 (m, 1H), 4.04 (dt, 1H), 3.83 (ddd, 1H), 3.28 − 3.27 (m, 1H), 3.05 − 2.95 (m, 3H), 2.01 − 1.84 (m, 2H), 1.50 (d, (6H). | 1.87 min, [MH]+ = 367 |
| 8.11 | 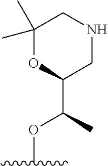 [SMILES: *O[C@H](C)[C@@H]1CNCC(C)(C)O1] | 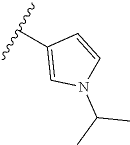 [SMILES: *c1ccn(C(C)C)c1] | 1H NMR (400 MHz, Methanol-d4) δ 8.76 (d, 1H), 8.66 (d, 1H), 7.69 (d, 1H), 7.56 (d, 1H), 7.45 (app t, 1H), 6.90 − 6.84 (m, 1H), 6.58 (dd, 1H), 4.68 (p, 1H), 4.34 (hept, 1H), 3.95 (ddd, 1H), 3.21 (dd, 1H), 2.67 (d, 1H), 2.60 − 2.48 (m, 2H), 1.50 (d, 6H), 1.44 (d, 3H), 1.34 (s, 3H), 1.14 (s, 3H). | 1.99 min, [MH]+ = 395 |
| 8.12 | 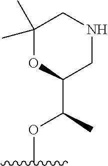 [SMILES: *O[C@H](C)[C@@H]1CNCC(C)(C)O1] | 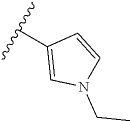 [SMILES: *c1ccn(CC)c1] | 1H NMR (400 MHz, Methanol-d4) δ 8.77 (d, 1H), 8.66 (d, 1H), 7.69 (d, 1H), 7.54 (d, 1H), 7.39 (app t, 1H), 6.81 (dd, 1H), 6.58 (dd, 1H), 4.66 (p, 1H), 4.01 (q, 2H), 3.95 (ddd, 1H), 3.21 (dd, 1H), 2.66 (d, 1H), 2.59 − 2.48 (m, 2H), 1.50 1.41 (m, 6H), 1.33 (s, 3H), 1.13 (s, 3H). | 1.92 min, [MH]+ = 381 |
| 8.13 | 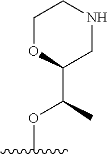 [SMILES: *O[C@H](C)[C@@H]1CNCCO1] | 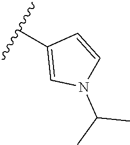 [SMILES: *c1ccn(C(C)C)c1] | 1H NMR (400 MHz, Methanol-d4) δ 8.77 (d, 1H), 8.66 (d, 1H), 7.69 (d, 1H), 7.54 (d, 1H), 7.48 − 7.43 (m, 1H), 6.88 (app t, 1H), 6.59 (app t, 1H), 4.79 (p, 1H), 4.35 (hept, 1H), 3.93 − 3.90 (m, 1H), 3.82 − 3.72 (m, 1H), 3.71 − 3.60 (m, 1H), 3.23 (dd, 1H), 2.87 − 2.71 (m, 3H), 1.50 (d, 6H), 1.46 (d, 3H). | 1.90 min, [MH]+ = 367 |
| 8.14 | 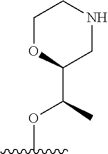 [SMILES: *O[C@H](C)[C@@H]1CNCCO1] | 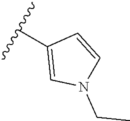 [SMILES: *c1ccn(CC)c1] | 1H NMR (400 MHz, Methanol-d4) δ 8.76 (d, 1H), 8.66 (d, 1H), 7.69 (d, 1H), 7.52 (d, 1H), 7.41 − 7.37 (m, 1H), 6.82 (app t, 1H), 6.58 (app t, 1H), 4.77 (p, 1H), 4.01 (q, 2H), 3.94 − 3.87 (m, 1H), 3.75 (ddd, 1H), 3.65 (ddd, 1H), 3.22 (dd, 1H), 2.87 − 2.69 (m, 3H), 1.54 − 1.39 (m, 6H). | 1.82 min, [MH]+ = 353 |
| 8.15 | 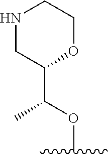 [SMILES: *O[C@H](C)[C@@H]1CNCCO1] | 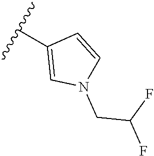 [SMILES: *c1ccn(CC(F)F)c1] | 1H NMR (400 MHz, Methanol-d4) δ 8.78 (d, 1H), 8.68 (d, 1H), 7.72 (d, 1H), 7.52 (d, 1H), 7.43 − 7.39 (m, 1H), 6.86 (app t, 1H), 6.66 (dd, 1H), 6.12 (tt, 1H), 4.78 (p, 1H), 4.38 (td, 2H), 3.91 (dt, 1H), 3.76 (ddd, 1H), 3.66 (ddd, 1H), 3.23 (dd, 1H), 2.88 − 2.71 (m, 3H), 1,46 (d, 3H). | 1.79 min, [MH]+ = 389 |
| 8.16 | 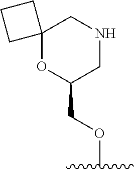 [SMILES: *OC[C@@H]1CNCC2(CCC2)O1] | 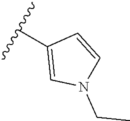 [SMILES: *c1ccn(CC)c1] | 1H NMR (400 MHz, Methanol-d4) δ 8.77 (d, 1H), 8.66 (dd, 1H), 7.68 (d, 1H), 7.50 (d, 1H), 7.40 (app t, 1H), 6.82 (app t, 1H), 6.60 (dd, 1H), 4.29 (dd, 1H), 4.21 (dd, 1H), 4.10 − 4.02 (m, 1H), 4.02 (q, 2H), 3.08 (dd, 1H), 2.99 (d, 1H), 2.70 (dd, 1H), 2.63 (dd, 1H), 2.34 − 2.24 (m, 1H), 2.19 − 2.03 (m, 2H), 1.94 − 1.76 (m, 2H), 1.72 − 1.55 (m, 1H), 1.46 (t, 3H). | 1.89 min, [MH]+ = 379 |
| 8.17 | 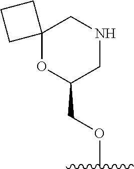 [SMILES: *OC[C@@H]1CNCC2(CCC2)O1] | 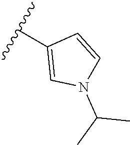 [SMILES: *c1ccn(C(C)C)c1] | 1H NMR (400 MHz, Methanol-d4) δ 8.78 (d, 1H), 8.67 (d, 1H), 7.70 (d, 1H), 7.52 (d, 1H), 7.47 (app t, 1H), 6.88 (dd, 1H), 6.60 (dd, 1H), 4.40 − 4.27 (m, 2H), 4.22 (dd, 1H), 4.06 (ddd, 1H), 3.08 (ddd, 1H), 2.99 (dd, 1H), 2.70 (dd, 1H), 2.63 (dd, 1H), 2.35 − 2.24 (m, 1H), 2.17 − 2.03 (m, 2H), 1.93 − 1.76 (m, 2H), 1.67 − 1.57 (m, 1H), 1.51 (d, (6H). | 1.96 min, [MH]+ = 393 |
| 8.18 | 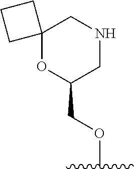 [SMILES: *OC[C@@H]1CNCC2(CCC2)O1] | 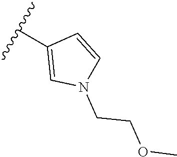 [SMILES: *c1ccn(CCOC)c1] | 1H NMR (400 MHz, Methanol-d4) δ 8.78 (d, 1H), 8.66 (d, 1H), 7.68 (d, 1H), 7.49 (d, 1H), 7.39 (app t, 1H), 6.84 (dd, 1H), 6.60 (dd, 1H), 4.29 (dd, 1H), 4.21 (dd, 1H), 4.13 (t, 2H), 4.06 (dddd, 1H), 3.71 (t, 2H), 3.35 (s, 3H), 3.07 (ddd, 1H), 2.98 (dd, 1H), 2.69 (dd, 1H), 2.63 (dd, 1H), 2.34 − 2.24 (m, 1H), 2.18 − 2.03 (m, 2H), 1.94 − 1.75 (m, 2H), 1.71 − 1.55 (m, 1H). | 1.82 min, [MH]+ = 409 |
| 8.19 | 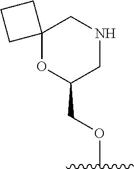 [SMILES: *OC[C@@H]1CNCC2(CCC2)O1] | 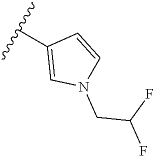 [SMILES: *c1ccn(CC(F)F)c1] | 1H NMR (400 MHz, Methanol-d4) δ 8.78 (d, 1H), 8.68 (d) 1H), 7.69 (d, 1H), 7.47 (d, 1H), 7.41 (app t, 1H), 6.87 (app t, 1H), 6.66 (dd, 1H), 6.13 (tt, 1H), 4.38 (td, 2H), 4.28 (dd, 1H), 4.20 (dd, 1H), 4.06 (dddd, 1H), 3.07 (ddd, 1H), 2.98 (dd, 1H), 2.69 (dd, 1H), 2.62 (dd, 1H), 2.34 − 2.23 (m, 1H), 2.17 − 2.02 (m, 2H), 1.93 − 1.75 (m, 2H), 1.70 − 1.50 (m, 1H). | 1.85 min, [MH]+ = 415 |
| 8.20 | 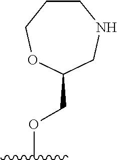 [SMILES: *OC[C@@H]1CNCCCO1] | 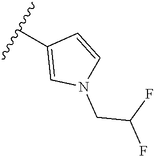 [SMILES: *c1ccn(CC(F)F)c1] | 1H NMR (400 MHz, Methanol-d4) δ 8.80 (d, 1H), 8.69 (d, 1H), 7.72 (d, 1H), 7.48 (d, 1H), 7.43 (app t, 1H), 6.87 (app t, 1H), 6.68 (dd, 1H), 6.12 (tt, 1H), 4.44 − 4.15 (m, 5H), 4.04 (ddd, 1H), 3.83 (ddd, 1H), 3.30 − 3.23 (m, 1H), 3.03 − 2.94 (m, 3H), 2.00 − 1.83 (m, 2H). | 1.40 min, [MH]+ = 389 |
| 8.21 | 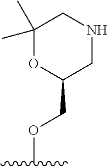 [SMILES: *OC[C@@H]1CNCC(C)(C)O1] | 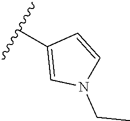 [SMILES: *c1ccn(CC)c1] | 1H NMR (400 MHz, Methanol-d4) δ 8.76 (d, 1H), 8.65 (d, 1H), 7.67 (d, 1H), 7.48 (d, 1H), 7.38 (app t, 1H), 6.81 (dd, 1H), 6.58 (dd, 1H), 4.33 − 4.22 (m, 2H), 4.21 − 4.11 (m, 1H), 4.01 (q, 2H), 3.16 (ddd, 1H), 2.74 − 2.55 (m, 3H), 1.45 (t, 3H), 1.37 (s, 3H), 1.18 (s, 3H). | 1.83 min, [MH]+ = 367 |
| 8.22 | 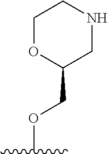 [SMILES: *OC[C@@H]1CNCCO1] | 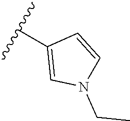 [SMILES: *c1ccn(CC)c1] | 1H NMR (400 MHz, Methanol-d4) δ 8.78 (d, 1H), 8.66 (d, 1H), 7.69 (d, 1H), 7.48 (d, 1H), 7.40 (app t, 1H), 6.82 (dd, 1H), 6.60 (dd, 1H), 4.32 (dd, 1H), 4.24 (dd, 1H), 4.10 − 4.03 (m, 1H), 4.01 (q, 2H), 3.91 (dd, 1H), 3.70 (td, 1H), 3.16 − 3.08 (m, 1H), 2.93 − 2.76 (m, 3H), 1.46 (t, 3H). | 1.72 min, [MH]+ = 339 |
| 8.23 | 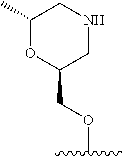 [SMILES: *OC[C@@H]1CNC[C@@H](C)O1] | 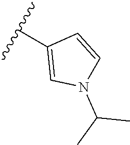 [SMILES: *c1ccn(C(C)C)c1] | 1H NMR (400 MHz, Methanol-d4) δ 8.71 (d, 1H), 8.59 (d, 1H), 7.63 (d, 1H), 7.45 − 7.42 (m, 2H), 6.85 (dd, 1H), 6.57 (dd, 1H), 4.53 (dd, 1H), 4.46 (dd, 1H), 4.31 (p, 1H), 4.26 4.18 (m, 1H), 4.10 − 3.98 (m, 1H), 3.06 (d, 2H), 2.96 (dd, 1H), 2.53 (dd, 1H), 1.48 (d, 6H), 1.15 (d, 3H). | 1.92 min, [MH]+ = 367 |
| 8.24 | 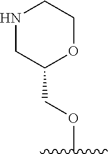 [SMILES: *OC[C@@H]1CNCCO1] | 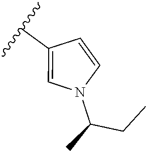 [SMILES: *c1ccn([C@H](C)CC)c1] | 1H NMR (400 MHz, Methanol-d4) δ 8.78 (d, 1H), 8.67 (d, 1H), 7.70 (d, 1H), 7.51 (d, 1H), 7.45 (app t, 1H), 6.85 (app t, 1H), 6.61 (dd, 1H), 4.33 (dd, 1H), 4.25 (dd, 1H), 4.12 − 3.99 (m, 2H), 3.96 − 3.87 (m, 1H), 3.71 (td, 1H), 3.14 (dd, 1H), 2.93 − 2.77 (m, 3H), 1.90 − 1.78 (m, 2H), 1.50 (d, 3H), 0.86 (t, 3H). | 1.91 min, [MH]+ = 367. |
| 8.25 | 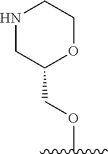 [SMILES: *OC[C@@H]1CNCCO1] | 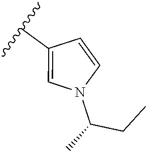 [SMILES: *c1ccn([C@@H](C)CC)c1] | 1H NMR (400 MHz, Methanol-d4) δ 8.79 (d, 1H), 8.67 (d, 1H), 7.71 (d, 1H), 7.51 (d, 1H), 7.46 (app t, 1H), 6.85 (dd, 1H), 6.62 (dd, 1H), 4.33 (dd, 1H), 4.26 (dd, 1H), 4.12 − 4.00 (m, 2H), 3.92 (dd, 1H), 3.72 (td, 1H), 3.14 (dd, 1H), 2.92 − 2.78 (m, 3H), 1.88 − 1.78 (m, 2H), 1.50 (d, 3H), 0.86 (t, 3H). | 1.91 min, [MH]+ = 367 |

Analytical data for quinoxalines synthesised by general method D:

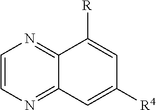

| | LC MS | |
|---|---|---|
| No | Method | Name |
| 8.1 | 2 | 7-[1-(2-methoxyethyl)-1H- |
| | | pyrrol-3-yl]-5-{[(5S)-4- |
| | | oxa-7-azaspiro[2.5]octan- |
| | | 5-yl]methoxy}quinoxaline |
| 8.2 | 2 | 5-{[(5S)-4-oxa-7- |
| | | azaspiro[2.5]octan-5- |
| | | yl]methoxy}- 7-[1-(propan- |
| | | 2-yl)-1H-pyrrol-3- |
| | | yl]quinoxaline |
| 8.3 | 2 | 5-{[(2S)-6,6- |
| | | dimethylmorpholin-2- |
| | | yl]methoxy}-7-[1-(propan- |
| | | 2-yl)-1H-pyrrol-3- |
| | | yl]quinoxaline |
| 8.4 | 2 | 5-{[(2S)-6,6- |
| | | dimethylmorpholin-2- |
| | | yl]methoxy}-7-[1-(2- |
| | | methoxyethyl)-1H-pyrrol- |
| | | 3-yl]quinoxaline |
| 8.5 | 2 | 7-[1-(2,2-difluoroethyl)- |
| | | 1H-pyrrol-3-yl]-5-{[(2S)- |
| | | 6,6-dimethylmorpholin-2- |
| | | yl]methoxy}quinoxaline |
| 8.6 | 2 | 5-{[(2S)-morpholin-2- |
| | | yl]methoxy}-7-[1-(propan- |
| | | 2-yl)-1H-pyrrol-3- |
| | | yl]quinoxaline |
| 8.7 | 2 | 7-[1-(2-methoxyethy])-1H- |
| | | pyrrol-3-yl]-5-{[(2S)- |
| | | morpholin-2- |
| | | yl]methoxy}quinoxaline |
| 8.8 | 2 | 7-[1-(2,2-difluoroethyl)- |
| | | 1H-pyrrol-3-yl]-5-{[(2S)- |
| | | morpholin-2- |
| | | yl]methoxy}quinoxaline |
| 8.9 | 2 | 7-(1-ethyl-1H-pyrrol-3-yl)- |
| | | 5-{[(2S)-1,4-oxazepan-2- |
| | | yl]methoxy}quinoxaline |
| 8.10 | 2 | 5-{[(2S)-1,4-oxazepan-2- |
| | | yl]methoxy}-7-[1-(propan- |
| | | 2-yl)-1H-pyrrol-3- |
| | | yl]quinoxaline |
| 8.11 | 2 | 5-[(1R)-1-[(2S)-6,6- |
| | | dimethylmorpholin-2- |
| | | yl]ethoxy]-7-[1-(propan- |
| | | 2-yl)-1H-pyrrol-3- |
| | | yl]quinoxaline |
| 8.12 | 2 | 5-[(1R)-1-[(2S)-6,6- |
| | | dimethylmorpholin-2- |
| | | yl]ethoxy]-7-(1-ethyl- |
| | | 1H-pyrrol-3- |
| | | yl)quinoxaline |
| 8.13 | 2 | 5-[(1R)-1-[(2S)- |
| | | morpholin-2-yl]ethoxy]-7- |
| | | [1-(propan-2-y])-1H- |
| | | pyrrol-3-yl]quinoxaline |
| 8.14 | 2 | 7-(1-ethyl-1H-pyrrol-3- |
| | | yl)-5-[(1R)-1-[(2S)- |
| | | morpholin-2- |
| | | yl]ethoxy]quinoxaline |
| 8.15 | 2 | 7-[1-(2,2-difluoroethyl)- |
| | | 1H-pyrrol-3-yl]-5-[(1R)- |
| | | 1-[(2S)-morpholin-2- |
| | | yl]ethoxy]quinoxaline |
| 8.16 | 2 | 7-(1-ethyl-1H-pyrrol-3- |
| | | yl)-5-{[(6S)-5-oxa-8- |
| | | azaspiro[3.5]nonan-6- |
| | | yl]methoxy}quinoxaline |
| 8.17 | 2 | 5-{[(6S)-5-oxa-8- |
| | | azaspiro[3.5]nonan-6- |
| | | yl]methoxy}-7-[1- |
| | | (propan-2-yl)-1H-pyrrol- |
| | | 3-yl]quinoxaline |
| 8.18 | 2 | 7-[1-(2-methoxyethyl)- |
| | | 1H-pyrrol-3-yl]-5- |
| | | {[(6S)-5-oxa-8- |
| | | azaspiro[3.5]nonan-6- |
| | | yl]methoxy}quinoxaline |
| 8.19 | 2 | 7-[1-(2,2-difluoroethyl)- |
| | | 1H-pyrrol-3-yl]-5- |
| | | {[(6S)-5-oxa-8- |
| | | azaspiro[3.5]nonan-6- |
| | | yl]methoxy}quinoxaline |
| 8.20 | 2 | 7-[1-(2,2-difluoroethyl)- |
| | | 1H-pyrrol-3-yl]-5- |
| | | {[(2S)-1,4-oxazepan-2- |
| | | yl]methoxy}quinoxaline |
| 8.21 | 2 | 5-{[(2S)-6,6- |
| | | dimethylmorpholin-2- |
| | | yl]methoxy}-7-(1-ethyl- |
| | | 1H-pyrrol-3- |
| | | yl)quinoxaline |
| 8.22 | 2 | 7-(1-ethyl-1H-pyrrol-3- |
| | | yl)-5-{[(2S)-morpholin- |
| | | 2- |
| | | yl]methoxy}quinoxaline |
| 8.23 | 2 | 5-{[(2S,6R)-6- |
| | | methylmorpholin-2- |
| | | yl]methoxy}-7-[1- |
| | | (propan-2-yl)-1H-pyrrol- |
| | | 3-yl]quinoxaline |
| 8.24 | 2 | 7-{1-[(2R)-butan-2-yl]- |
| | | 1H-pyrrol-3-yl}-5- |
| | | {[(2S)-morpholin-2- |
| | | yl]methoxy}quinoxaline |
| 8.25 | 2 | 7-{1-[(2S)-butan-2-yl]- |
| | | 1H-pyrrol-3-yl}-5- |
| | | {[(2S)-morpholin-2- |
| | | yl]methoxy}quinoxaline |

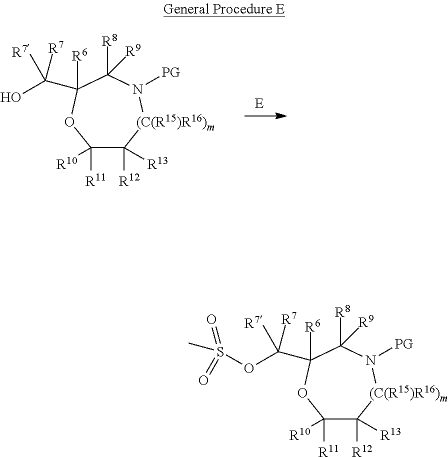

Triethylamine (2.5 eq) and methanesulfonyl chloride (1.2 eq) were added to a solution of the alcohol (1.0 eq) in dichloromethane (0.1-0.2 M) at room temperature. After 2-15 hours, the reaction mixture was partitioned between water and dichloromethane. The aqueous phase was extracted with dichloromethane and the combined organic phases were washed with brine, dried over anhydrous sodium/magnesium sulfate, and concentrated. The crude material was purified using purification method 1.

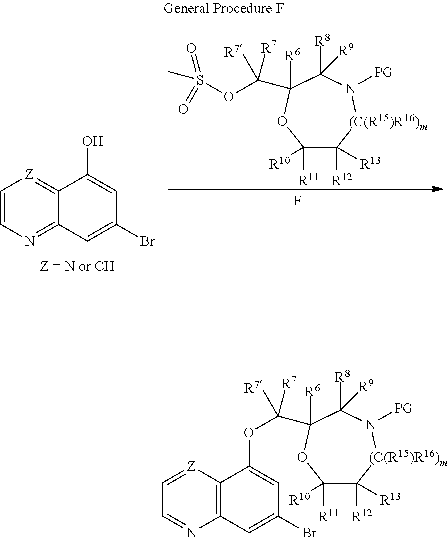

To a solution of heteroaryl bromide (7-bromoquinoxalin-5-ol or 7-bromoquinolin-5-ol) (1.0 eq) and mesylated alcohol (1.1 eq) in the appropriate solvent (dimethyl sulfoxide or N,N-dimethylformamide) (0.1-0.4 M) was added cesium carbonate (3.0 eq). The reaction mixture was heated at 100° C. for 4-16 hours. The cooled reaction mixture was partitioned between ethyl acetate and water. The aqueous layer was extracted with ethyl acetate and the combined organics were washed with brine, dried over anhydrous sodium/magnesium sulfate and concentrated in vacuo. The residue was purified using purification method 1.

TABLE 9
Analytical data for intermediates synthesised by general method F

| | | | LC MS | LC MS |
|---|---|---|---|---|
| No | Structure | 1H NMR | RT, m/z | Method |
| 9.1 | 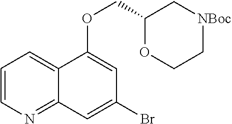 [SMILES: CC(C)(C)OC(=O)N1CCO[C@H](COc2cc(Br)cc3ncccc23)C1] | 1H NMR (400 MHz, Methanol-d4) δ 8.82 (dd, 1H), 8.61 (d, 1H), 7.77 (dd, 1H), 7.51 (dd, 1H), 7.16 (d, 1H), 4.30 − 4.18 (m, 2H), 4.17 − 4.08 (m, 1H), 4.01 − 3.80 (m, 3H), 3.61 (td, 1H), 3.17 − 2.82 (m, 2H) 1.47 (s, 9H). | 3.03 min, [MH]+ = 423/425 | 2 |
| 9.2 | 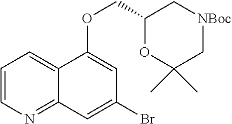 [SMILES: CC(C)(C)OC(=O)N1C[C@@H](COc2cc(Br)cc3ncccc23)OC(C)(C)C1] | 1H NMR (400 MHz, Methanol-d4) δ 8.83 (dd, 1H), 8.62 (br s, 1H), 7.78 (dd, 1H), 7.53 (dd, 1H), 7.18 (d, 1H), 4.31 − 4.11 (m, 4H), 3.81 (d, 1H), 3.03 − 2.59 (m, 2H), 1.48 (s, 9H), 1.28 (s, 3H), 1.23 (s, 3H). | 3.34 min, [MH]+ = 451/453 | 2 |
| 9.3 | 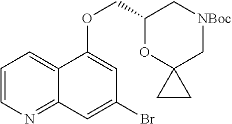 [SMILES: CC(C)(C)OC(=O)N1C[C@@H](COc2cc(Br)cc3ncccc23)OC2(CC2)C1] | 1H NMR (400 MHz, Methanol-d4) δ 8.82 (dd, 1H), 8.60 (d, 1H), 7.77 (dd, 1H), 7.52 (dd, 1H), 7.15 (d, 1H), 4.34 − 4.14 (m, 3H), 4.12 − 4.01 (m, 1H), 3.65 − 3.37 (m, 2H), 3.24 − 2.97 (m, 1H), 1.47 (s, 9H), 0.95 − 0.86 (m, 1H), 0.82 − 0.68 (m, 2H), 0.66 − 0.56 (m, 1H). | 3.26 min, [MH]+ = 449/451 | 2 |
| 9.4 | 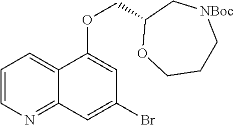 [SMILES: CC(C)(C)OC(=O)N1CCCO[C@H](COc2cc(Br)cc3ncccc23)C1] | 1H NMR (400 MHz, Methanol-d4) δ 8.88 − 8.79 (m, 1H), 8.65 (dd, 1H), 7.78 (s, 1H), 7.53 (dd, 1H), 7.17 (d, 1H), 4.28 − 4.05 (m, 4H), 3.97 (td, 1H), 3.75 − 3.57 (m, 2H), 3.52 − 3.33 (m, 2H), 2.00 − 1.84 (m, 2H), 1.46 (d, 9H). | 3.05 min, [MH]+ = 337/339 | 2 |
| 9.5 | 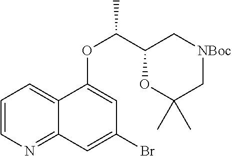 [SMILES: C[C@@H](Oc1cc(Br)cc2ncccc12)[C@@H]1CN(C(=O)OC(C)(C)C)CC(C)(C)O1] | 1H NMR (400 MHz, Chloroform-d) δ 8.86 (dd, 1H), 8.49 (d, 1H), 7.85 (d, 1H), 7.35 (ddd, 1H), 7.04 (br s, 1H), 4.52 − 4.18 (m, 3H), 3.92 − 3.59 (m, 2H), 2.66 (dd, 1H), 1.54 − 1.35 (m, 12H), 1.23 (s, 3H), 1.19 (s, 3H). | 3.53 min, [MH]+ = 465/467 | 2 |
| 9.6 | 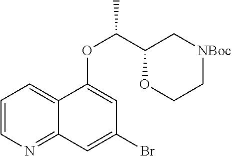 [SMILES: C[C@@H](Oc1cc(Br)cc2ncccc12)[C@@H]1CN(C(=O)OC(C)(C)C)CCO1] | 1H NMR (600 MHz, Methanol-d4) δ 8.83 (d, 1H), 8.63 (d, 1H), 7.78 (s, 1H), 7.52 (dd, 1H), 7.24 (s, 1H), 4.72 (p, 1H), 4.26 − 4.00 (m, 1H), 3.97 − 3.89 (m, 1H), 3.86 − 3.76 (m, 1H), 3.63 (ddd, 1H), 3.57 (td, 1H), 3.03 (ddd, 1H), 2.98 − 2.81 (m, 1H), 1.50 − 1.32 (m, 12H). | 3.16 min, [MH]+ = 437/439 | 2 |
| 9.7 | 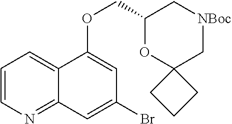 [SMILES: CC(C)(C)OC(=O)N1C[C@@H](COc2cc(Br)cc3ncccc23)OC2(CCC2)C1] | No NMR recorded. | 3.41 min, [MH]+ = 463/465 | 2 |
| 9.8 | 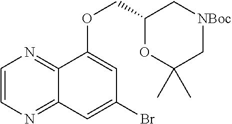 [SMILES: CC(C)(C)OC(=O)N1C[C@@H](COc2cc(Br)cc3nccnc23)OC(C)(C)C1] | 1H NMR (400 MHz, Methanol-d4) δ 8.90 (d, 1H), 8.86 (d, 1H), 7.87 (d, 1H), 7.49 (d, 1H), 4.33 − 4.16 (m, 4H), 3.79 (d, 1H), 3.02 − 2.55 (m, 2H), 1.48 (s, 9H), 1.28 (s, 3H), 1.23 − 1.21 (m, 3H). | 3.15 min, [MH]+ = 452/454 | 2 |
| 9.9 | 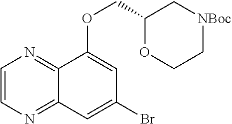 [SMILES: CC(C)(C)OC(=O)N1CCO[C@H](COc2cc(Br)cc3nccnc23)C1] | 1H NMR (400 MHz, Chloroform-d) δ 8.87 − 8.81 (m, 2H), 7.91 (d, 1H), 7.26 (s, 1H), 4.31 (dd, 1H), 4.23 (dd, 1H), 4.20 − 4.09 (m, 1H), 4.06 − 3.99 (m, 1H), 3,99 − 3.85 (m, 2H), 3.64 (td, 1H), 3.08 − 2.84 (m, 2H), 1.47 (s, 9H). | 2.85 min, [MH]+ = 424/426 | 2 |
| 9.10 | 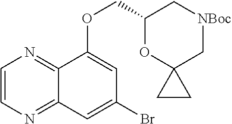 [SMILES: CC(C)(C)OC(=O)N1C[C@@H](COc2cc(Br)cc3nccnc23)OC2(CC2)C1] | 1H NMR (400 MHz, Chloroform-d) δ 8.84 − 8.82 (m, 2H), 7.90 (d, 1H), 7.25 (d, 1H), 4.36 − 4.10 (m, 4H), 3.52 − 3.21 (m, 2H), 3.15 − 2.96 (m, 1H), 1.45 (s, 9H), 1.01 − 0.88 (m, 1H), 0.78 − 0.61 (m, 3H). | 3.08 min, [MH]+ = 450/452 | 2 |
| 9.11 | 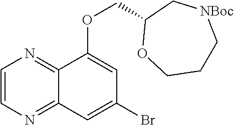 [SMILES: CC(C)(C)OC(=O)N1CCCO[C@H](COc2cc(Br)cc3nccnc23)C1] | 1H NMR (400 MHz, Methanol-d4) mixture of rotamers: δ 8.90 (d, 1H), 8.85 (d, 1H), 7.86 (s, 1H), 7.44 (s, 1H), 4.34 − 4.19 (m, 2H), 4.19 − 4.00 (m, 2H), 3.97 − 3.84 (m, 1H), 3.69 − 3.53 (m, 2H), 3.54 − 3.37 (m, 2H), 1.96 − 1.79 (m, 2H), 1.52 − 1.37 (m, 9H). | 2.88 min, [MH]+ = 438/440 | 2 |
| 9.12 | 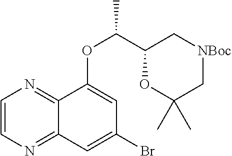 [SMILES: C[C@@H](Oc1cc(Br)cc2nccnc12)[C@@H]1CN(C(=O)OC(C)(C)C)CC(C)(C)O1] | 1H NMR (400 MHz, Methanol-d4) δ 8.88 (d, 1H), 8.85 (d, 1H), 7.85 (d, 1H), 7.54 (br s, 1H), 4.69 (br s, 1H), 4.27 (d, 1H), 3.93 (ddd, 1H), 3.74 (d, 1H), 2.80 (dd, 1H), 2.76 − 2.53 (m, 1H), 1.47 − 1.43 (m, 12H), 1.23 (s, 3H), 1.15 (s, 3H) | 3.36 min, [MH]+ = 466/468 | 2 |
| 9.13 | 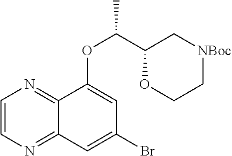 [SMILES: C[C@@H](Oc1cc(Br)cc2nccnc12)[C@@H]1CN(C(=O)OC(C)(C)C)CCO1] | 1H NMR (400 MHz, Methanol-d4) δ 8.88 (d, 1H), 8.84 (d, 1H), 7.83 (d, 1H), 7.49 (d, 1H), 4.78 (p, 1H), 4.13 (br s, 1H), 3.94 − 3.87 (m, 1H), 3.83 − 3.70 (m, 1H), 3.66 (ddd, 1H), 3.56 (td, 1H), 3.16 − 2.82 (m, 2H), 1.46 (d, 3H), 1.38 (br s, 9H). | 3.00 min, [MH]+ = 438/440 | 2 |
| 9.14 | 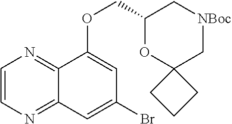 [SMILES: CC(C)(C)OC(=O)N1C[C@@H](COc2cc(Br)cc3nccnc23)OC2(CCC2)C1] | 1H NMR (600 MHz, Chloroform-d) δ 8.87 − 8.81 (m, 2H), 7.91 (s, 1H), 7.33 (s, 1H), 4.37 − 4.25 (m, 1H), 4.23 (dd, 1H), 4.19 − 4.07 (m, 1H), 4.07 − 3.93 (m, 2H), 2.91 − 2.59 (m, 2H), 2.20 − 2.05 (m, 2H), 2.03 − 1.88 (m, 2H), 1.88 − 1.79 (m, 1H), 1.73 − 1.63 (m, 1H), 1.50 − 1.42 (m, 9H). | 3.22 min, [MH]+ = 464/466 | |
| 9.15 | 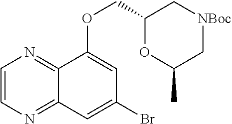 [SMILES: C[C@@H]1CN(C(=O)OC(C)(C)C)C[C@@H](COc2cc(Br)cc3nccnc23)O1] | 1H NMR (600 MHz, Methanol-d4) δ 8.88 (s, 1H), 8.83 (s, 1H), 7.80 (s, 1H), 7.42 (s, 1H), 4.45 − 4.26 (m, 3H), 4.08 − 3.83 (m, 1H), 3.83 − 3.43 (m, 3H), 3.25 − 2.81 (m, 1H), 1.52 − 1.38 (m, 3H), 1.31 − 1.10 (m, 9H). | 2.89 min, [MH]+ = 438/440 | 2 |
| 9.16 | 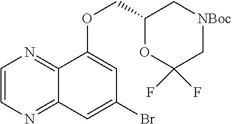 [SMILES: CC(C)(C)OC(=O)N1C[C@@H](COc2cc(Br)cc3nccnc23)OC(F)(F)C1] | 1H NMR (400 MHz, Chloroform-d) δ 8.88 − 8.79 (m, 2H), 7.94 (d, 1H), 7.26 (d, 1H), 4.70 − 4.59 (m, 1H), 4.49 − 4.29 (m, 3H), 4.26 − 4.18 (m, 1H), 3.41 − 3.13 (m, 2H), 1.46 (s, 9H). | 3.05 min, [MH]+ = 460/462 | 2 |
| 9.17 | 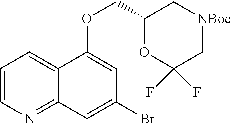 [SMILES: CC(C)(C)OC(=O)N1C[C@@H](COc2cc(Br)cc3ncccc23)OC(F)(F)C1] | 1H NMR (400 MHz, Chloroform-d) mixture of rotamers δ 8.95 − 8.87 (m, 1H), 8.54 − 8.44 (m, 1H), 8.03 − 7.90 (m, 1H), 7.48 − 7.39 (m, 1H), 6.96 (d, 1H), 4.65 − 4.55 (m, 1H), 4.42 − 4.20 (m, 4H), 3.37 − 3.11 (m, 2H), 1.56 − 1.41 (m, 9H). | 3.19 min, [MH]+ = 459/461 | 2 |
| 9.18 | 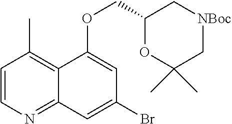 [SMILES: Cc1ccnc2cc(Br)cc(OC[C@@H]3CN(C(=O)OC(C)(C)C)CC(C)(C)O3)c12] | 1H NMR (400 MHz, Methanol-d4) δ 8.59 (d, 1H), 7.75 (d, 1H), 7.29 (dd, 1H), 7.13 (d, 1H), 4.27 − 4.08 (m, 4H), 3.83 (d, 1H), 2.96 (d, 3H), 2.93 − 2.62 (m, 2H), 1.49 (s, 9H), 1.28 (s, 3H), 1.24 (s, 3H). | 3.28 min, [MH]+ = 465/467 | 2 |
| 9.19 | 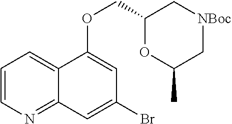 [SMILES: C[C@@H]1CN(C(=O)OC(C)(C)C)C[C@@H](COc2cc(Br)cc3ncccc23)O1] | 1H NMR (400 MHz, Chloroform-d) δ 8.89 (dd, 1H), 8.52 (d, 1H), 7.90 (s, 1H), 7.39 (dd, 1H), 6.97 (d, 1H), 4.39 − 4.15 (m, 3H), 4.04 − 3.89 (m, 1H), 3.86 − 3.37 (m, 3H), 3.25 − 2.88 (m, 1H), 1.54 − 1.31 (m, 9H), 1.27 − 1.22 (m, 3H). | 3.09 min, [MH]+ = 437/439 | 2 |

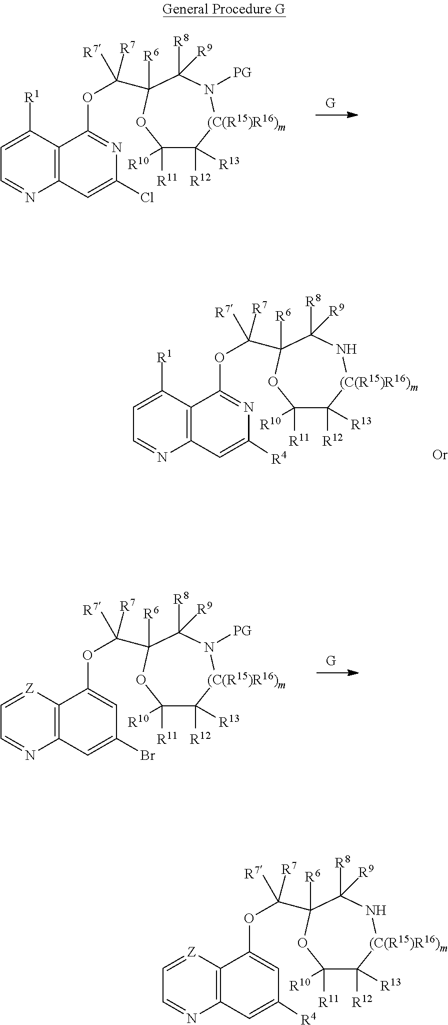

To a degassed solution of the appropriate heteroaryl halide (1 eq) and arylboronic acid/ester (1.1-1.25 eq) in 1,4-dioxane/water (0.1-0.5 M in a 4:1 ratio) was added potassium phosphate tribasic (3.0 eq) followed by [1,1′-bis(diphenylphosphino)ferrocene]dichloropalladium(II) complex with dichloromethane (Pd(dppf)Cl₂·CH₂Cl₂) (5-10 mol %) The reaction mixture was sealed and heated to 90-120° C. over 30 minutes to 41 hours. The cooled reaction mixture was diluted with 2 M aqueous sodium hydroxide solution or saturated brine and extracted with ethyl acetate. The organic layer was dried over anhydrous sodium/magnesium sulfate and concentrated. The residue was either purified by standard purification method 1, 2 or 3 or taken through crude and the protecting group removed using either of the following conditions:

To a solution of protected intermediate (1.0 eq) in dichloromethane (0.05-0.2 M) was added trifluoroacetic acid (6.0-60 eq). The reaction mixture was stirred at room temperature for 1-24 hours. On consumption of starting material the reaction mixture was purified using one of the standard purification methods.

TABLE 10
Analytical data for naphthyridines synthesised by general method G:

| | | | | LC MS RT, |
|---|---|---|---|---|
| No | R | R⁴ | 1H NMR | m/z |
| 10.1 | 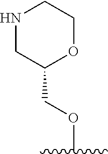 [SMILES: *OC[C@@H]1CNCCO1] | 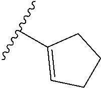 [SMILES: *C1=CCCC1] | 1H NMR (600 MHz, Methanol-d4) δ 8.94 (dd, 1H), 8.62 (d, 1H), 7.55 (dd, 1H), 7.29 (s, 1H), 6.87 (app t, 1H), 4.63 − 4.55 (m, 2H), 4.08 − 4.00 (m, 1H), 3.93 (ddd, 1H), 3.74 − 3.66 (m, 1H), 3.07 (dd, 1H), 2.92 − 2.74 (m, 5H), 2.62 (tt, 2H), 2.13 (p, 2H). | 1.76 min, [MH]+ = 312 |
| 10.2 | 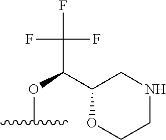 [SMILES: *O[C@H]([C@@H]1CNCCO1)C(F)(F)F] | 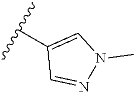 [SMILES: *c1cnn(C)c1] | 1H NMR (600 MHz, Methanol-d4) δ 8.99 (dd, 1H), 8.65 (dd, 1H), 8.31 (s, 1H), 8.12 (s, 1H), 7.70 (s, 1H), 7.57 (dd, 1H), 6.45 (qd, 1H), 4.19 − 4.14 (m, 1H), 3.97 (s, 3H), 3.94 (d, 1H), 3.68 (ddd, 1H), 3.05 (dd, 1H), 2.80 − 2.70 (m, 3H). | 1.77 min, [MH]+ = 394 |
| 10.3 | 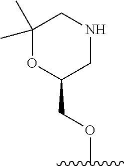 [SMILES: *OC[C@@H]1CNCC(C)(C)O1] | 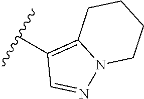 [SMILES: *c1cnn2c1CCCC2] | 1H NMR (600 MHz, Methanol-d4) δ 8.87 (dd, 1H), 8.45 (dd, 1H), 8.00 (s, 1H), 7.46 − 7.41 (m, 2H), 4.53 (dd, 1H), 4.38 (dd, 1H), 4.32 − 4.24 (m, 1H), 4.15 (t, 2H), 3.23 − 3.17 (m, 2H), 3.17 − 3.12 (m, 1H), 2.83 (d, 1H), 2.71 (dd, 2H), 2.11 − 2.04 (m, 2H), 1,97 − 1.92 (m, 3H), 1.40 (s, 3H), 1.24 (s, 3H). | 1.75 min, [MH]+ = 394 |
| 10.4 | 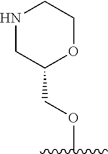 [SMILES: *OC[C@@H]1CNCCO1] | 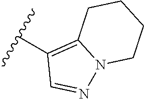 [SMILES: *c1cnn2c1CCCC2] | 1H NMR (600 MHz, Methanol-d4) δ 8.82 (dd, 1H), 8.40 (dd, 1H), 7.94 (s, 1H), 7.38 (dd, 1H), 7.35 (s, 1H), 4.46 (dd, 1H), 4.41 (dd, 1H), 4.12 (t, 2H), 4.00 − 3.90 (m, 2H), 3.74 − 3.65 (m, 1H), 3.14 (t, 2H), 3.06 (dd, 1H), 2.93 − 2.82 (m, 2H), 2.78 (dd, 1H), 2.08 − 2.01 (m, 2H), 1.95 − 1.86 (m, 2H). | 1.62 min, [MH]+ = 366 |
| 10.5 | 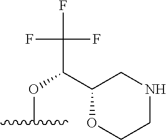 [SMILES: *O[C@@H]([C@@H]1CNCCO1)C(F)(F)F] | 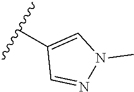 [SMILES: *c1cnn(C)c1] | 1H NMR (600 MHz, Methanol-d4) δ 8.99 (dd, 1H), 8.58 (dd, 1H), 8.28 (s, 1H), 8.10 (s, 1H), 7.72 (s, 1H), 7.57 (dd, 1H), 6.39 (p, 1H), 4.14 (ddd, 1H), 3.97 (s, 3H), 3.92 (d, 1H), 3.68 (td, 1H), 3.08 (d, 1H), 2.90 (dd, 1H), 2.87 − 2.77 (m, 2H). | 1.76 min, [MH]+ = 394 |
| 10.6 | 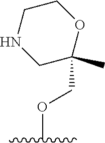 [SMILES: *OC[C@]1(C)CNCCO1] | 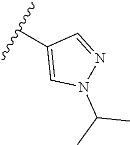 [SMILES: *c1cnn(C(C)C)c1] | 1H NMR (600 MHz, Methanol-d4) δ 8.93 (dd, 1H), 8.61 (dd, 1H), 8.37 (s, 1H), 8.15 (s, 1H), 7.60 (s, 1H), 7.51 (dd, 1H), 4.86 (d, 1H), 4.62 (p, 1H), 4.58 (d, 1H), 3.87 − 3.82 (m, 1H), 3.81 − 3.74 (m, 1H), 3.06 (d, 1H), 2.88 − 2.79 (m, 3H), 1.58 (d, 6H), 1.46 (s, 3H). | 1.66 min, [MH]+ = 368 |
| 10.7 | 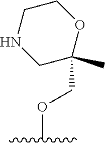 [SMILES: *OC[C@]1(C)CNCCO1] | 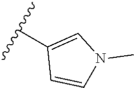 [SMILES: *c1ccn(C)c1] | 1H NMR (600 MHz, Methanol-d4) δ 8.86 (dd, 1H), 8.57 (dd, 1H), 7.49 − 7.45 (m, 2H), 7.43 (dd, 1H), 6.75 − 6.70 (m, 1H), 6.70 − 6.65 (m, 1H), 4.79 (d, 1H), 4.62 (d, 1H), 3.91 − 3.82 (m, 1H), 3.81 − 3.76 (m, 1H), 3.74 (s, 3H), 3.04 (d, 1H), 2.89 − 2.80 (m, 3H), 1.45 (s, 3H). | 1.58 min, [MH]+ = 368 |
| 10.8 | 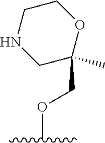 [SMILES: *OC[C@@]1(C)CNCCO1] | 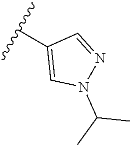 [SMILES: *c1cnn(C(C)C)c1] | 1H NMR (600 MHz, Methanol-d4) δ 8.92 (dd, 1H), 8.60 (d, 1H), 8.36 (s, 1H), 8.14 (s, 1H), 7.59 (s, 1H), 7.50 (dd, 1H), 4.85 (d, 1H), 4.65 − 4.61(m, 1H), 4.57 (d, 1H), 3.91 − 3.81 (m, 1H), 3.80 − 3.74 (m, 1H), 3.05 (d, 1H), 2.88 − 2.79 (m, 3H), 1.58 (d, 6H), 1.45 (s, 3H). | 1.68 min, [MH]+ = 368 |
| 10.9 | 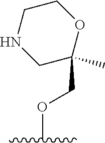 [SMILES: *OC[C@@]1(C)CNCCO1] | 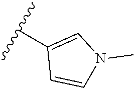 [SMILES: *c1ccn(C)c1] | 1H NMR (600 MHz, Methanol-d4) δ 8.87 (dd, 1H), 8.58 (dd, 1H), 7.49 − 7.46 (m, 2H), 7.44 (dd, 1H), 6.75 − 6.71 (m, 1H), 6.70 − 6.65 (m, 1H), 4.80 (d, 1H), 4.62 (d, 1H), 3,92 − 3.84 (m, 1H), 3.81 − 3.76 (m, 1H), 3.75 (s, 3H), 3,04 (d, 1H), 2.90 − 2.80 (m, 3H), 1.45 (s, 3H). | 1.58 min, [MH]+ = 339 |
| 10.10 | 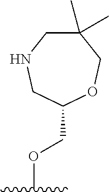 [SMILES: *OC[C@@H]1CNCC(C)(C)CO1] | 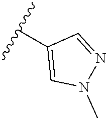 [SMILES: *c1cnn(C)c1] | 1H NMR (400 MHz, Methanol-d4) δ 8.85 (dd, 1H), 8.47 (ddd, 1H), 8.14 (app t, 1H), 8.00 (d, 1H), 7.47 (d, 1H), 7.43 (dd, 1H), 4.54 (dd, 1H), 4.47 (dd, 1H), 4.10 (ddt, 1H), 3.93 (s, 3H), 3.64 (d, 1H), 3.44 (d, 1H), 3.19 (dd, 1H), 2.86 (dd, 1H), 2.69 (d, 1H), 2.58 (d, 1H), 0.97 (s, 3H), 0.87 (s, 3H). | 1.65 min, [MH]+ = 368 |
| 10.11 | 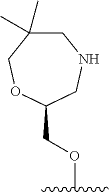 [SMILES: *OC[C@@H]1CNCC(C)(C)CO1] | 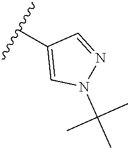 [SMILES: *c1cnn(C(C)(C)C)c1] | 1H NMR (400 MHz, Methanol-d4) δ 8.92 (dd, 1H), 8.59 (ddd, 1H), 8.37 (d, 1H), 8.12 (d, 1H), 7.62 (d, 1H), 7.51 (dd, 1H), 4.70 (dd, 1H), 4.60 (dd, 1H), 4.27 (ddt, 1H), 3.75 (d, 1H), 3.49 (d, 1H), 3.40 (dd, 1H), 3.14 (dd, 1H), 2.93 (d, 1H), 2.79 (d, 1H), 1.66 (s, 9H), 1.05 (s, 3H), 0.94 (s, 3H). | 1.86 min, [MH]+ = 410 |
| 10.12 | 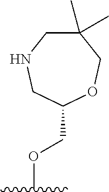 [SMILES: *OC[C@@H]1CNCC(C)(C)CO1] | 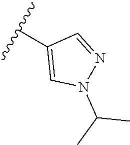 [SMILES: *c1cnn(C(C)C)c1] | 1H NMR (400 MHz, Chloroform-d) δ 8.93 (dd, 1H), 8.45 (ddd, 1H), 8.03 (d, 1H), 7.98 (d, 1H), 7.59 (d, 1H), 7.32 (dd, 1H), 4.64 − 4.43 (m, 3H), 4.12 − 4.04 (m, 1H), 3.65 (d, 1H), 3.47 (d, 1H), 3.29 − 3.22 (m, 1H), 2.97 − 2.80 (m, 1H), 2.73 2.61 (m, 2H), 1.57 (d, 6H), 0.99 (s, 3H), 0.86 (s, 3H). | 1.77 min, [MH]+ = 396 |
| 10.13 | 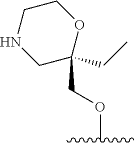 [SMILES: *OC[C@@]1(CC)CNCCO1] | 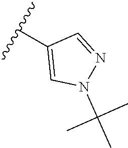 [SMILES: *c1cnn(C(C)(C)C)c1] | 1H NMR (400 MHz, Methanol-d4) δ 8.90 (dd, 1H), 8.54 (ddd, 1H), 8.39 (d, 1H), 8.14 (d, 1H), 7.59 (d, 1H), 7.47 (dd, 1H), 4.90 − 4.82 (m, 1H), 4.61 (d, 1H), 3.83 (ddd, 1H), 3.71 (ddd, 1H), 3.01 (d, 1H), 2.89 − 2.76 (m, 3H), 2.03 (dq, 1H), 1.76 (dq, 1H), 1.66 (s, 9H), 0.98 (t, 3H). | 1.83 min, [MH]+ = 396 |
| 10.14 | 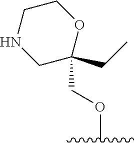 [SMILES: *OC[C@]1(CC)CNCCO1] | 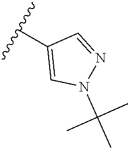 [SMILES: *c1cnn(C(C)(C)C)c1] | 1H NMR (400 MHz, Methanol-d4) δ 8.90 (dd, 1H), 8.55 (ddd, 1H), 8.39 (d, 1H), 8.14 (d, 1H), 7.60 (d, 1H), 7.48 (dd, 1H), 4.90 − 4.82 (m, 1H), 4.61 (d, 1H), 3.83 (ddd, 1H), 3.71 (ddd, 1H), 3.01 (d, 1H), 2.89 − 2.81 (m, 3H), 2.03 (dq, 1H), 1.76 (dq, 1H), 1.66 (s, 9H), 0.98 (t, 3H). | 1.83 min, [MH]+ = 396 |
| 10.15 | 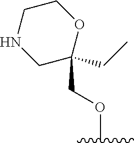 [SMILES: *OC[C@@]1(CC)CNCCO1] | 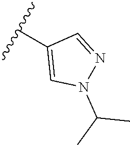 [SMILES: *c1cnn(C(C)C)c1] | 1H NMR (400 MHz, Methanol-d4) δ 8.91 (dd, 1H), 8.56 (ddd, 1H), 8.35 (d, 1H), 8.13 (d, 1H), 7.59 (d, 1H), 7.49 (dd, 1H), 4.91 − 4.84 (m, 1H), 4.68 − 4.54 (m, 2H), 3.83 (ddd, 1H), 3.71 (ddd, 1H), 3.02 (d, 1H), 2.90 − 2.76 (m, 3H), 2.10 − 1.96 (m, 1H), 1.76 (dq, 1H), 1.56 (d, 6H), 0.98 (t, 3H). | 1.76 min, [MH]+ = 382 |
| 10.16 | 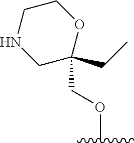 [SMILES: *OC[C@]1(CC)CNCCO1] | 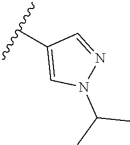 [SMILES: *c1cnn(C(C)C)c1] | 1H NMR (400 MHz, Methanol-d4) δ 8.90 (dd, 1H), 8.54 (ddd, 1H), 8.33 (d, 1H), 8.12 (d, 1H), 7.57 (d, 1H), 7.47 (dd, 1H), 4.90 − 4.83 (m, 1H), 4.68 − 4.53 (m, 2H), 3.83 (ddd, 1H), 3.70 (ddd, 1H), 3.01 (d, 1H), 2.88 − 2.80 (m, 3H), 2.10 − 1.96 (m, 1H), 1.76 (dq, 1H), 1.56 (d, 6H), 0.98 (t, 3H), | 1.75 min, [MH]+ = 382 |

Analytical data for naphthyridines synthesised by general method G:

| | LC MS | Name |
|---|---|---|
| No | Method | |
| 10.1 | 2 | 7-(cyclopent-1-en-1-yl)-5- |
| | | {[(2S)-morpholin-2- |
| | | yl]methoxy}-1,6- |
| | | naphthyridine |
| 10.2 | 2 | 7-(1-methyl-1H-pyrazol-4- |
| | | yl)-5-[(1R)-2,2,2-trifluoro- |
| | | 1-[(2S)-morpholin-2- |
| | | yl]ethoxy]-1,6- |
| | | naphthyridine |
| 10.3 | 2 | 5-{[(2S)-6,6- |
| | | dimethylmorpholin-2- |
| | | yl]methoxy}-7- |
| | | (4H,5H,6H,7H- |
| | | pyrazolo[1,5-a]pyridin-3- |
| | | yl}-1,6-naphthyridine |
| 10.4 | 2 | 5-{[(2S)-morpholin-2- |
| | | yl]methoxy}-7- |
| | | {4H,5H,6H,7H- |
| | | pyrazolo[1,5-a]pyridin-3- |
| | | yl}-1,6-naphthyridine |
| 10.5 | 2 | 7-(1-methyl-1H-pyrazol-4- |
| | | yl)-5-[(1S)-2,2,2-trifluoro- |
| | | 1-[(2S)-morpholin-2- |
| | | yl]ethoxy]-1,6- |
| | | naphthyridine |
| 10.6 | 2 | 5-{[(2S)-2- |
| | | methylmorpholin-2- |
| | | yl]methoxy}-7-[1-(propan- |
| | | 2-yl)-1H-pyrazol-4-yl]-1,6- |
| | | naphthyridine |
| 10.7 | 2 | 7-(1-methyl-1H-pyrrol~3- |
| | | yl)-5-{[(2S)-2- |
| | | methylmorpholin-2- |
| | | yl]methoxy}-1,6- |
| | | naphthyridine |
| 10.8 | 2 | 5-{[(2R)-2- |
| | | methylmorpholin-2- |
| | | yl]methoxy}-7-[1-(propan- |
| | | 2-yl)-1H-pyrazol-4-yl]-1,6- |
| | | naphthyridine |
| 10.9 | 2 | 7-(1-methyl-1H-pyrrol-3- |
| | | yl)-5-{[(2R)-2- |
| | | methylmorpholin-2- |
| | | yl]methoxy}-1,6- |
| | | naphthyridine |
| 10.10 | 2 | 5-{[(2S)-6,6-dimethyl-1,4- |
| | | oxazepan-2-yl]methoxy}- |
| | | 7-(1-methyl-1H-pyrazol-4- |
| | | yl)-1,6-naphthyridine |
| 10.11 | 2 | 7-(1-tert-butyl-1H-pyrazol- |
| | | 4-yl)-5-{[(2S)-6,6- |
| | | dimethyl-1,4-oxazepan-2- |
| | | yl]methoxy}-1,6- |
| | | naphthyridine |
| 10.12 | 2 | 5-{[(2S)-6,6-dimethyl-1,4- |
| | | oxazepan-2-yl]methoxy}- |
| | | 7-[1-(propan-2-yl)-1H- |
| | | pyrazol-4-yl]-1,6- |
| | | naphthyridine |
| 10.13 | 2 | 5-{[(2R)-6,6- |
| | | dimethylmorpholin-2- |
| | | yllmethoxy}-7-[1-(3- |
| | | methyloxolan-3-yl)-1H- |
| | | pyrazol-4-yl]-1,6- |
| | | naphthyridine |
| 10.14 | 2 | 7-(1-tert-butyl-1H-pyrazol- |
| | | 4-yl)-5-{[(2S)-2- |
| | | ethylmorpholin-2- |
| | | yl]methoxy}-1,6- |
| | | naphthyridine |
| 10.15 | 2 | 7-(1-tert-butyl-1H-pyrazol- |
| | | 4-yl)-5-{[(2R)-2- |
| | | ethylmorpholin-2- |
| | | yl]methoxy}-1,6- |
| | | naphthyridine |
| 10.16 | 2 | 5-{[(2S)-2- |
| | | ethylmorpholin-2- |
| | | yl]methoxy}-7-[1-(propan- |
| | | 2-yl)-1H-pyrazol-4-yl]-1,6- |
| | | naphthyridine |

TABLE 11
Analytical data for quinolines synthesised by general method G:

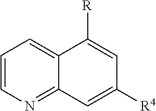

| | | | | LC MS | LC MS | |
|---|---|---|---|---|---|---|
| No | R | R⁴ | 1H NMR | RT, m/z | Method | Name |
| 11.1 | 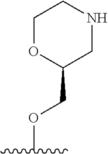 [SMILES: *OC[C@@H]1CNCCO1] | 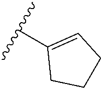 [SMILES: *C1=CCCC1] | 1H NMR (600 MHz, Methanol-d4) δ 8.77 (dd, 1H), 8.60 (d, 1H), 7.50 (s, 1H), 7.44 (dd, 1H), 7.22 (d, 1H), 6.51 (t, 1H), 4.25 (dd, 1H), 4.21 (dd, 1H), 4.08 − 4.02 (m, 1H), 3.97 (d, 1H), 3.75 (td, 1H), 3.16 (dd, 1H), 2.99 − 2.87 (m, 3H), 2.87 − 2.80 (m, 2H), 2.65 − 2.57 (m, 2H), 2.14 − 2.05 (m, 2H). | 1.56 min, [MH]+ = 311 | 2 | 7-(cyclopent-1-en- 1-yl)-5- {[(2S)-morpholin-2- yl]methoxy}quinoline |
| 11.2 | 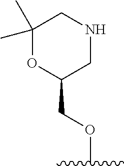 [SMILES: *OC[C@@H]1CNCC(C)(C)O1] | 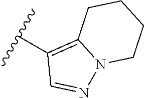 [SMILES: *c1cnn2c1CCCC2] | 1H NMR (600 MHz, Methanol-d4) δ 8.78 (dd, 1H), 8.60 (dd, 1H), 7.88 (s, 1H), 7.59 (s, 1H), 7.44 (dd, 1H), 7.13 (s, 1H), 4.27 − 4.22 (m, 1H), 4.22 − 4.17 (m, 3H), 4.14 (dd, 1H), 3.14 − 3.05 (m, 3H), 2.73 (d, 1H), 2.69 − 2.60 (m, 2H), 2.16 − 2.09 (m, 2H), 1.99 − 1.93 (m, 2H), 1.39 (s, 3H), 1.20 (s, 3H). | 1.57 min, [MH]+ = 393 | 2 | 5-{[(2S)-6,6- dimethylmorpholin-2- yl]methoxy}-7- {4H,5H,6H,7H- pyrazolo[1,5- a]pyridin-3- yl}quinoline |
| 11.3 | 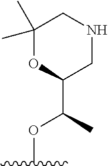 [SMILES: *O[C@H](C)[C@@H]1CNCC(C)(C)O1] | 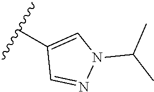 [SMILES: *c1cnn(C(C)C)c1] | 1H NMR (400 MHz, Methanol-d4) δ 8.77 (dd, 1H), 8.58 (ddd, 1H), 8.27 (d, 1H), 8.01 (d, 1H), 7.74 (app t, 1H), 7.41 (dd, 1H), 7.34 (d, 1H), 4.78 (p, 1H), 4.58 (hept, 1H), 4.08 (ddd, 1H), 3.42 − 3.34 (m, 1H), 3.03 − 2.95 (m, 1H), 2.85 (dd, 1H), 2.75 (d, 1H), 1.55 (d, 6H), 1.46 (d, 3H), 1.38 (s, 3H), 1.23 (s, 3H). | 1.71 min, [MH]+ = 395 | 2 | 5-[(1R)-1-[(2S)-6,6- dimethylmorpholin-2- yl]ethoxy]-7- [1-(propan-2- yl)-1H-pyrazol-4- yl]quinoline |
| 11.4 | 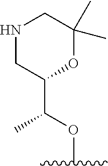 [SMILES: *O[C@H](C)[C@@H]1CNCC(C)(C)O1] | 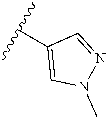 [SMILES: *c1cnn(C)c1] | 1H NMR (400 MHz, Methanol-d4) δ 8.80 − 8.75 (m, 1H), 8.64 − 8.57 (m, 1H), 8.20 − 8.14 (m, 1H), 8.02 − 7.98 (m, 1H), 7.74 − 7.69 (m, 1H), 7.45 − 7.40 (m, 1H), 7.32 − 7.27 (m, 1H), 4.67 (p, 1H), 3.98 − 3.90 (m, 4H), 3.14 (dd, 1H), 2.72 − 2.51 (m, 3H), 1.44 (d, 3H), 1.33 (s, 3H), 1.15 (s, 3H). | 1.49 min, [MH]+ = 367 | 2 | 5-[(1R)-1-[(2S)-6,6- dimethylmorpholin- 2- yl]ethoxy]-7- (1-methyl- 1H-pyrazol-4- yl)quinoline |
| 11.5 | 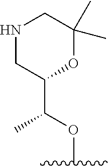 [SMILES: *O[C@H](C)[C@@H]1CNCC(C)(C)O1] | 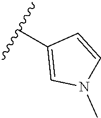 [SMILES: *c1ccn(C)c1] | 1H NMR (400 MHz, Methanol-d4) δ 8.71 (dd, 1H), 8.59 − 8.52 (m, 1H), 7.65 (app t, 1H), 7.35 (ddd, 1H), 7.29 − 7.21 (m, 2H), 6.73 (app t, 1H), 6.56 (dd, 1H), 4.60 (p, 1H), 3.93 (ddd, 1H), 3.72 (s, 3H), 3.14 (dd, 1H), 2.67 (d, 1H), 2.62 − 2.50 (m, 2H), 1.43 (d, 3H), 1.33 (s, 3H), 1.16 (s, 3H). | 1.55 min, [MH]+ = 366 | 2 | 5-[(1R)-1-[(2S)-6,6- dimethylmorpholin-2- yl]ethoxy]- 7-(1-methyl- 1H-pyrrol-3-yl) quinoline |
| 11.6 | 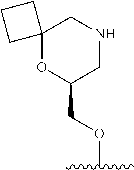 [SMILES: *OC[C@@H]1CNCC2(CCC2)O1] | 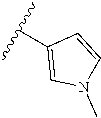 [SMILES: *c1ccn(C)c1] | 1H NMR (400 MHz, Methanol-d4) δ 8.71 (dd, 1H), 8.54 (ddd, 1H), 7.65 (app t, 1H), 7.35 (dd, 1H), 7.25 (app t, 1H), 7.18 (d, 1H), 6.74 − 6.70 (m, 1H), 6.56 (dd, 1H), 4.21 (dd, 1H), 4.15 (dd, 1H), 4.03 − 3.96 (m, 1H), 3.72 (s, 3H), 3.05 − 2.97 (m, 2H), 2.71 (dd, 1H), 2.63 (dd, 1H), 2.36 − 2.27 (m, 1H), 2.16 − 1.99 (m, 2H), 1.94 − 1.78 (m, 2H), 1.70 − 1.57 (m, 1H). | 1.52 min, [MH]+ = 364 | 2 | 7-(1-methyl- 1H-pyrrol-3- yl)-5-{[(6S)-5-oxa-8- azaspiro[3.5]nonan-6- yl]methoxy}quinoline |
| 11.7 | 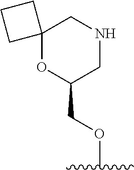 [SMILES: *OC[C@@H]1CNCC2(CCC2)O1] | 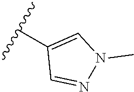 [SMILES: *c1cnn(C)c1] | 1H NMR (400 MHz, Methanol-d4) δ 8.76 (dd, 1H), 8.56 (ddd, 1H), 8.13 (d, 1H), 7.99 (d, 1H), 7.68 (dd, 1H), 7.41 (dd, 1H), 7.16 (d, 1H), 4.21 (dd, 1H), 4.16 (dd, 1H), 4.05 − 3.97 (m, 1H), 3.95 (s, 3H), 3.06 − 2.98 (m, 2H), 2.72 (dd, 1H), 2.64 (dd, 1H), 2.38 − 2.27 (m, 1H), 2.15 − 2.01 (m, 2H), 1.95 − 1.78 (m, 2H), 1.72 − 1.58 (m, 1H). | 1.46 min, [MH]+ = 365 | 2 | 7-(1-methyl- 1H-pyrazol-4- yl)-5-{[(6S)-5-oxa-8- azaspiro[3.5]nonan-6- yl]methoxy}quinoline |
| 11.8 | 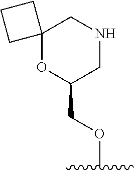 [SMILES: *OC[C@@H]1CNCC2(CCC2)O1] | 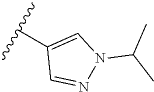 [SMILES: *c1cnn(C(C)C)c1] | 1H NMR (400 MHz, Methanol-d4) δ 8.76 (dd, 1H), 8.57 (ddd, 1H), 8.26 (d, 1H), 8.02 (d, 1H), 7.71 (dd, 1H), 7.41 (dd, 1H), 7.22 (d, 1H), 4.59 (hept, 1H), 4.23 (dd, 1H), 4.19 (dd, 1H), 4.06 − 3.98 (m, 1H), 3.06 − 2.98 (m, 2H), 2.73 (dd, 1H), 2.64 (dd, 1H), 2.37 − 2.27 (m, 1H), 2.16 − 2.00 (m, 2H), 1.95 − 1.77 (m, 2H), 1.70 − 1.58 (m, 1H), 1.56 (d, 6H). | 1.60 min, [MH]+ = 393 | 2 | 5-{[(6S)-5-oxa-8- azaspiro[3.5]nonan-6- yl]methoxy}- 7-[1-(propan- 2-yl)-1H-pyrazol-4- yl]quinoline |
| 11.9 | 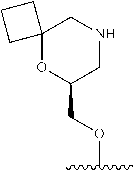 [SMILES: *OC[C@@H]1CNCC2(CCC2)O1] | 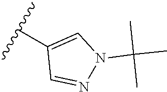 [SMILES: *c1cnn(C(C)(C)C)c1] | 1H NMR (400 MHz, Methanol-d4) δ 8.77 (dd, 1H), 8.58 (ddd, 1H), 8.34 (d, 1H), 8.03 (d, 1H), 7.73 (t, 1H), 7.44 − 7.38 (m, 1H), 7.25 (d, 1H), 4.25 (dd, 1H), 4.20 (dd, 1H), 4.06 − 3.98 (m, 1H), 3.06 − 2.97 (m, 2H), 2.73 (dd, 1H), 2.64 (dd, 1H), 2.38 − 2.27 (m, 1H), 2.15 − 2.01 (m, 2H), 1.95 − 1.78 (m, 2H), 1.70 − 1.58 (m, 10H). | 1.67 min, [MH]+ = 407 | 2 | 7-(1-tert-butyl-1H- pyrazol-4-yl)- 5-{[(6S)-5- oxa-8-azaspiro [3.5]nonan- 6-yl]methoxy} quinoline |
| 11.10 | 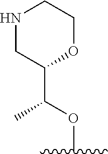 [SMILES: *O[C@H](C)[C@@H]1CNCCO1] | 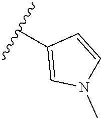 [SMILES: *c1ccn(C)c1] | 1H NMR (600 MHz, Methanol-d4) δ 8.70 (dd, 1H), 8.52 (dd, 1H), 7.65 (s, 1H), 7.33 (dd, 1H), 7.26 − 7.20 (m, 2H), 6.71 (app t, 1H), 6.55 (app t, 1H), 4.68 (p, 1H), 3.95 − 3.87 (m, 1H), 3.73 − 3.68 (m, 4H), 3.65 (td, 1H), 3.14 (dd, 1H), 2.87 − 2.71 (m, 3H), 1.43 (d, 3H). | 1.42 min, [MH]+ = 338 | 2 | 7-(1-methyl- 1H-pyrrol-3- yl)-5-[(1R)-1-[(2S)- morpholin-2- yl]ethoxy]quinoline |

TABLE 12
Analytical data for quinoxalines synthesised by general method G:

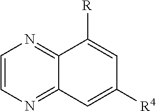

| | | | | LC MS RT, | LC MS | |
|---|---|---|---|---|---|---|
| No | R | R⁴ | 1H NMR | m/z | Method | Name |
| 12.1 | 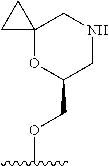 [SMILES: *OC[C@@H]1CNCC2(CC2)O1] | 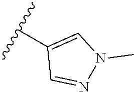 [SMILES: *c1cnn(C)c1] | 1H NMR (600 MHz, Methanol- d4) δ 8.91 − 8.85 (m, 1H), 8.79 − 8.76 (m, 1H), 8.25 (s, 1H), 8.07 (s, 1H), 7.83 (d, 1H), 7.51 (s, 1H), 4.39 − 4.34 (m, 1H), 4.34 − 4.26 (m, 2H), 3.99 (s, 3H), 3.46 (d, 1H), 3.41 − 3.35 (m, 2H), 3.11 (dd, 1H), 2.57 (d, 1H), 0.99 − 0.90 (m, 2H), 0.75 − 0.66 (m, 2H). | 1.63 min, [MH]+ = 352 | 2 | 7-(1-methyl-1H-pyrazol-4- yl)-5-{[(5S)-4-oxa-7- azaspiro[2.5]octan-5- yl]methoxy}quinoxaline |
| 12.2 | 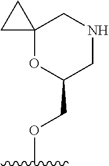 [SMILES: *OC[C@@H]1CNCC2(CC2)O1] | 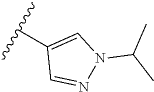 [SMILES: *c1cnn(C(C)C)c1] | 1H NMR (600 MHz, Methanol-d4) δ 8.87 (d, 1H), 8.78 − 8.75 (m, 1H), 8.36 (s, 1H), 8.09 (s, 1H), 7.83 (d, 1H), 7.54 (d, 1H), 4.62 (hept, 1H), 4.35 (dd, 1H), 4.31 − 4.23 (m, 2H), 3.37 − 3.33 (m, 1H), 3.24 (d, 1H), 2.91 (dd, 1H), 2.38 (d, 1H), 1.58 (d, 6H), 0.92 − 0.84 (m, 2H), 0.64 − 0.61 (m, 2H). | 1.75 min, [MH]+ = 380 | 2 | 5-{[(5S)-4-oxa-7- azaspiro[2.5]octan-5- yl]methoxy}-7-[1-(propan- 2-yl)-1H-pyrazol-4- yl]quinoxaline |
| 12.3 | 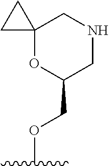 [SMILES: *OC[C@@H]1CNCC2(CC2)O1] | 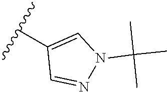 [SMILES: *c1cnn(C(C)(C)C)c1] | 1H NMR (600 MHz, Methanol-d4) δ 8.88 − 8.85 (m, 1H), 8.76 (d, 1H), 8.43 (s, 1H), 8.09 (s, 1H), 7.84 (d, 1H), 7.57 (d, 1H), 4.36 (dd, 1H), 4.31 − 4.19 (m, 2H), 3.37 − 3.33 (m, 1H), 3.26 − 3.21 (m, 1H), 2.93 (dd, 1H), 2.39 (d, 1H), 1.68 (s, 9H), 0.93 − 0.85 (m, 2H), 0.64 − 0.60 (m, 2H). | 1.85 min, [MH]+ = 394 | 2 | 7-(1-tert-butyl-1H-pyrazol- 4-yl)-5-{[(5S)-4-oxa-7- azaspiro[2.5]octan-5- yl]methoxy}quinoxaline |
| 12.4 | 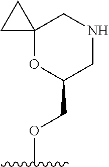 [SMILES: *OC[C@@H]1CNCC2(CC2)O1] | 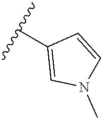 [SMILES: *c1ccn(C)c1] | 1H NMR (400 MHz, Methanol- d4) δ 8.78 (d, 1H), 8.66 (d, 1H), 7.67 (d, 1H), 7.44 (d, 1H), 7.30 (app t, 1H), 6.73 (dd, 1H), 6.58 (dd, 1H), 4.34 − 4.18 (m, 3H), 3.73 (s, 3H), 3.36 − 3.31 (m, 1H), 3.26 (dd, 1H), 2.99 − 2.88 (m, 1H), 2.45 − 2.37 (m, 1H), 0.92 − 0.85 (m, 2H), 0.71 − 0.58 (m, 2H). | 1.79 min, [MH]+ = 351 | 2 | 7-(1-methyl-1H-pyrrol-3- yl)-5-{[(5S)-4-oxa-7- azaspiro[2.5]octan-5- yl]methoxy}quinoxaline |
| 12.5 | 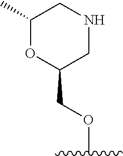 [SMILES: *OC[C@@H]1CNC[C@@H](C)O1] | 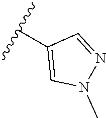 [SMILES: *c1cnn(C)c1] | 1H NMR (400 MHz, Methanol- d4) δ 8.79 (d, 1H), 8.69 (d, 1H), 8.18 (d, 1H), 8.01 (d, 1H), 7.71 (d, 1H), 7.43 (d, 1H), 4.56 (dd, 1H), 4.48 (dd, 1H), 4.29 − 4.20 (m, 1H), 4.11 − 3.99 (m, 1H), 3.95 (s, 3H), 3.08 (d, 2H), 2.97 (dd, 1H), 2.55 (dd, 1H), 1.16 (d, 3H) | 1.58 min, [MH]+ = 340 | 2 | 7-(1-methyl-1H-pyrazol-4- yl)-5-{[(2S,6R)-6- methylmorpholin-2- yl]methoxy}quinoxaline |
| 12.6 | 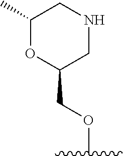 [SMILES: *OC[C@@H]1CNC[C@@H](C)O1] | 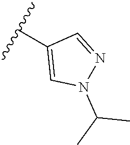 [SMILES: *c1cnn(C(C)C)c1] | 1H NMR (400 MHz, Methanol- d4) δ 8.77 (d, 1H), 8.66 (d, 1H), 8.29 (d, 1H), 8.02 (d, 1H), 7.71 (d, 1H), 7.44 (d, 1H), 4.63 − 4.54 (m, 2H), 4.48 (dd, 1H), 4.29 − 4.20 (m, 1H), 4.10 − 4.00 (m, 1H), 3.09 (d, 2H), 2.98 (dd, 1H), 2.56 (dd, 1H), 1,55 (d, 6H), 1.16 (d, 3H). | 1.73 min, [MH]+ = 368 | 2 | 5-{[(2S,6R)-6- methylmorpholin-2- yl]methoxy}-7-[1-(propan- 2-yl)-1H-pyrazol-4- yl]quinoxaline |
| 12.7 | 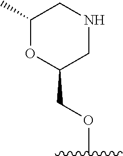 [SMILES: *OC[C@@H]1CNC[C@@H](C)O1] | 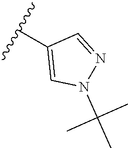 [SMILES: *c1cnn(C(C)(C)C)c1] | 1H NMR (400 MHz, Methanol- d4) δ 8.76 (d, 1H), 8.65 (d, 1H), 8.36 (d, 1H), 8.03 (d, 1H), 7.70 (d, 1H), 7.45 (d, 1H), 4.55 (dd, 1H), 4.47 (dd, 1H), 4.28 − 4.19 (m, 1H), 4.09 − 3.97 (m, 1H), 3.07 (d, 2H), 2.96 (dd, 1H), 2.53 (dd, 1H), 1.64 (s, 9H), 1.15 (d, 3H). | 1.81 min, [MH]+ = 382 | 2 | 7-(1-tert-butyl-1H-pyrazol- 4-yl)-5-{[(2S,6R)-6- methylmorpholin-2- yl]methoxy}quinoxaline |
| 12.8 | 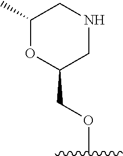 [SMILES: *OC[C@@H]1CNC[C@@H](C)O1] | 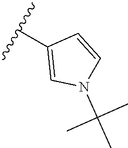 [SMILES: *c1ccn(C(C)(C)C)c1] | 1H NMR (400 MHz, Methanol- d4) δ 8.76 (d, 1H), 8.65 (d, 1H), 7.70 (d, 1H), 7.58 (dd, 1H), 7.54 (d, 1H), 6.99 (dd, 1H), 6.62 (dd, 1H), 4.58 (dd, 1H), 4.52 (dd, 1H), 4.28 − 4.23 (m, 1H), 4.10 − 3.98 (m, 1H), 3.06 (d, 2H), 2.95 (dd, 1H), 2.53 (dd, 1H), 1.60 (s, 9H), 1.16 (d, 3H). | 1.99 min, [MH]+ = 381 | 2 | 7-(1-tert-butyl-1H-pyrrol- 3-yl)-5-{[(2S,6R)-6- methylmorpholin-2- yl]methoxy}quinoxaline |

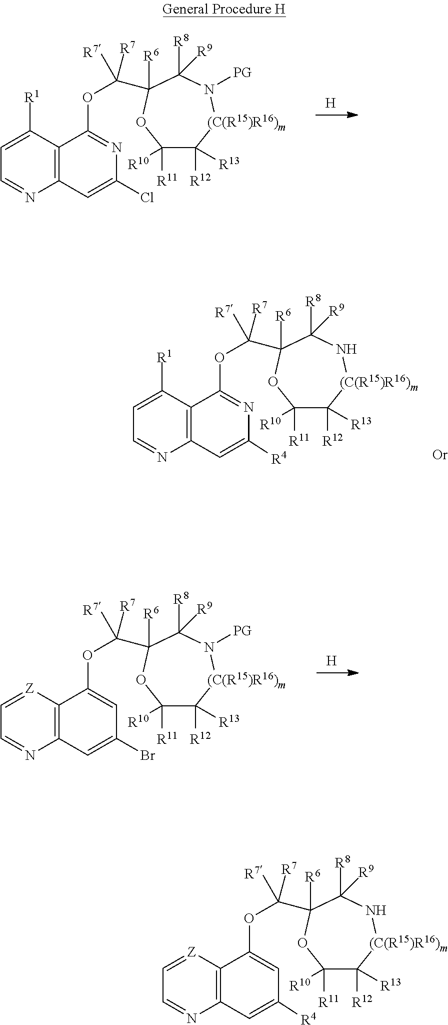

To a degassed solution of heteroaryl halide (1.0 eq) and arylboronic acid/ester (1.1-1.25 eq) in tetrahydrofuran/2 M aqueous sodium carbonate solution (0.1-0.5 M in a 10:1 ratio) was added (2-dicyclohexylphosphino-2′,4′,6′-triisopropyl-1,1′-biphenyl)[2-(2′-amino-1,1′-biphenyl)]palladium(II) methanesulfonate (XPhos Pd G3) (2-10 mol %). The reaction mixture was sealed and heated to 135° C. for 1 hour. The cooled reaction mixture was diluted with 2 M aqueous sodium hydroxide solution and extracted with ethyl acetate. The organic layer was washed with brine, dried over anhydrous sodium/magnesium sulfate and concentrated. The residue was either purified by standard purification method 1, 2 or 3 or taken through as the crude product and the protecting group removed using either of the following conditions:

Conditions 1: To a solution of protected intermediate (1.0 eq) in dichloromethane (0.05-0.2 M) was added trifluoroacetic acid (6.0-60 eq). The reaction mixture was stirred at room temperature for 1-24 hours. On consumption of starting material the reaction mixture was purified using one of the standard purification methods.

Conditions 2: A solution of intermediate (10.0 eq) in 1,4-dioxane/water (1:3 ratio, 0.05-0.2 M) was heated at 140-170° C. by microwave irradiation for 1-2 hours. The solvents were removed under reduced pressure and the reaction mixture was purified using one of the standard purification methods.

TABLE 13
Analytical data for compounds synthesised by general method H:

| | | | LC MS RT, | LC MS | Name |
|---|---|---|---|---|---|
| No | Structure | 1H NMR | m/z | Method | |
| 13.1 | 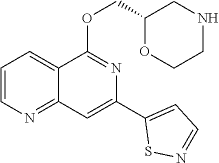 [SMILES: c1cnc2cc(-c3ccns3)nc(OC[C@@H]3CNCCO3)c2c1] | 1H NMR (400 MHz, DMSO-d6) δ 9.12 (dd, 1H), 8.66 (d, 1H), 8.56 (app dt, 1H), 8.22 − 8.15 (m, 2H), 7.68 (dd, 1H), 4.52 (d, 2H), 3.93 − 3.87 (m, 1H), 3.81 − 3.77 (m, 1H), 3.59 − 3.44 (m, 1H), 3.01 (dd, 1H), 2.77 − 2.60 (m, 3H). | 1.60 min, [MH]+ = 329 | 2 | 5-{[(2S)-morpholin-2- yl]methoxy}-7-(1,2- thiazol-5-yl)-1,6- naphthyridine |
| 13.2 | 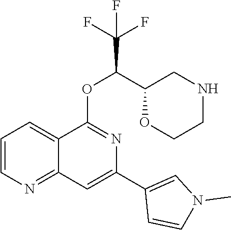 [SMILES: Cn1ccc(-c2cc3ncccc3c(O[C@H]([C@@H]3CNCCO3)C(F)(F)F)n2)c1] | 1H NMR (600 MHz, DMSO-d6) δ 9.00 (dd, 1H), 8.42 (dd, 1H), 7.65 − 7.59 (m, 2H), 7.50 (dd, 1H), 6.80 (app t, 1H), 6.72 (app t, 1H), 6.52 − 6.45 (m, 1H), 4.06 (dd, 1H), 3.84 (dd, 1H), 3.69 (s, 3H), 3.57 − 3.50 (m, 1H), 2.99 − 2.93 (m, 1H), 2.67 (d, 1H), 2.64 − 2.56 (m, 1H), 2.53 (d, 1H). | 1.88 min, [MH]+ = 393 | 2 | 7-(1-methyl-1H-pyrrol-3- yl)-5-[(1R)-2,2,2- trifluoro-1-[(2S)- morpholin-2-yl]ethoxy]- 1,6-naphthyridine |
| 13.3 | 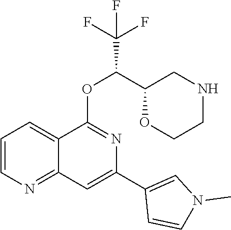 [SMILES: Cn1ccc(-c2cc3ncccc3c(O[C@@H]([C@@H]3CNCCO3)C(F)(F)F)n2)c1] | 1H NMR (600 MHz, DMSO-d6) δ 9.01 (dd, 1H), 8.43 (dd, 1H), 7.64 (s, 1H), 7.57 (d, 1H), 7.50 (dd, 1H), 6.81 (app t, 1H), 6.72 − 6.68 (m, 1H), 6.39 − 6.31 (m, 1H), 4.09 − 4.00 (m, 2H), 3.88 − 3.82 (m, 1H), 3.70 (s, 3H), 3.61 − 3.54 (m, 2H), 3.04 − 2.95 (m, 1H), 2.81 − 2.66 (m, 2H). | 1.86 min, [MH]+ = 393 | 2 | 7-(1-methyl-1H-pyrrol-3- yl)-5-[(1S)-2,2,2- trifluoro-1-[(2S)- morpholin-2-yl]ethoxy]- 1,6-naphthyridine |
| 13.4 | 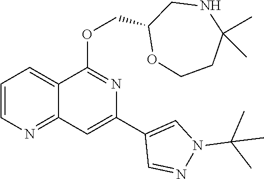 [SMILES: CC1(C)CCO[C@H](COc2nc(-c3cnn(C(C)(C)C)c3)cc3ncccc23)CN1] | 1H NMR (400 MHz, Methanol- d4) δ 8.92 (dd, 1H), 8.59 (ddd, 1H), 8.37 (d, 1H), 8.12 (d, 1H), 7.61 (d, 1H), 7.50 (dd, 1H), 4.65 (dd, 1H), 4.54 (dd, 1H), 4.20 − 4.11 (m, 1H), 3.94 (ddd, 1H), 3.82 (ddd, 1H), 3.12 − 2.96 (m, 2H), 1.96 − 1.79 (m, 2H), 1.23 (s, 3H), 1.16 (s, 3H). | 1.79 min, [MH]+ = 410 | 2 | 7-(1-tert-butyl-1H- pyrazol-4-yl)-5-{[(2S)- 5,5-dimethyl-1,4- oxazepan-2-yl]methoxy}- 1,6-naphthyridine |
| 13.5 | 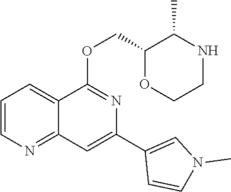 [SMILES: C[C@@H]1NCCO[C@@H]1COc1nc(-c2ccn(C)c2)cc2ncccc12] | 1H NMR (400 MHz, Chloroform- d) δ 8.89 (dd, 1H), 8.43 (ddd, 1H), 7.56 (d, 1H), 7.32 (app t, 1H), 7.27 (dd, 1H), 6.69 (dd, 1H), 6.64 (app t, 1H), 4.64 (dd, 1H), 4.58 (dd, 1H), 4.22 − 4.16 (m, 1H), 3.95 (dt, 1H), 3.71 (s, 3H), 3.71 − 3.63 (m, 1H), 3.28 − 3.21 (m, 1H), 3.18 (ddd, 1H), 2.72 (dt, 1H), 1.29 (d, 3H). | 1.59 min, [MH]+ = 339 | 2 | 7-(1-methyl-1H-pyrrol-3- yl)-5-{[(2S,3S)-3- methylmorpholin-2- yl]methoxy}-1,6- naphthyridine |
| 13.6 | 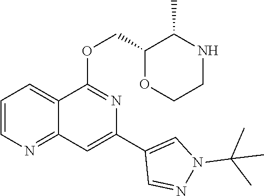 [SMILES: C[C@@H]1NCCO[C@@H]1COc1nc(-c2cnn(C(C)(C)C)c2)cc2ncccc12] | 1H NMR (400 MHz, Chloroform- d) δ 8.92 (dd, 1H), 8.46 (ddd, 1H), 8.09 − 8.04 (m, 2H), 7.60 (d, 1H), 7.32 (dd, 1H), 4.65 (dd, 1H), 4.57 (dd, 1H), 4.19 (ddd, 1H), 3.95 (dt, 1H), 3.67 (ddd, 1H), 3.24 (ddd, 1H), 3.18 (ddd, 1H), 2.71 (dt, 1H), 1.65 (s, 9H), 1.28 (d, 3H). | 1.77 min, [MH]+ = 382 | 2 | 7-(1-tert-butyl-1H- pyrazol-4-yl)-5- {[(2S,3S)-3- methylmorpholin-2- yl]methoxy}-1,6- naphthyridine |
| 13.7 | 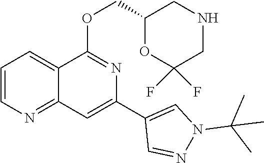 [SMILES: CC(C)(C)n1cc(-c2cc3ncccc3c(OC[C@@H]3CNCC(F)(F)O3)n2)cn1] | 1H NMR (600 MHz, Methanol- d4) δ 8.92 (dd, 1H), 8.60 − 8.55 (m, 1H), 8.37 (s, 1H), 8.12 (s, 1H), 7.61 (s, 1H), 7.50 (dd, 1H), 4.76 − 4.70 (m, 2H), 4.68 − 4.61 (m, 1H), 3.25 (dd, 1H), 3.16 (ddd, 1H), 3.01 − 2.88 (m, 2H), 1.66 (s, 9H). | 1.88 min, [MH]+ = 361 | 2 | 5-{[(2S)-2- ethylmorpholin-2- yl]methoxy}-7-[1- (propan-2-yl)-1H- pyrazol-4-yl]-1,6- naphthyridine |
| 13.8 | 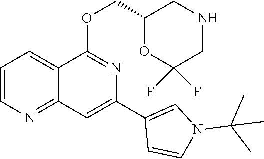 [SMILES: CC(C)(C)n1ccc(-c2cc3ncccc3c(OC[C@@H]3CNCC(F)(F)O3)n2)c1] | 1H NMR (600 MHz, Methanol- d4) δ 8.85 (dd, 1H), 8.54 (dd, 1H), 7.64 (app t, 1H), 7.48 (s, 1H), 7.42 (dd, 1H), 6.97 (app t, 1H), 6,68 (dd, 1H), 4.76 − 4.70 (m, 2H), 4.68 − 4.61 (m, 1H), 3.24 (dd, 1H), 3.18 − 3.13 (m, 1H), 2.99 − 2.86 (m, 2H), 1.60 (s, 9H). | 1.60 min, [MH]+ = 360 | 2 | 5-{[(2S)-6,6- difluoromorpholin-2- yl]methoxy}-7-(1-methyl- 1H-pyrrol-3- yl)quinoxaline |
| 13.9 | 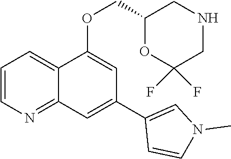 [SMILES: Cn1ccc(-c2cc(OC[C@@H]3CNCC(F)(F)O3)c3cccnc3c2)c1] | 1H NMR (400 MHz, Methanol- d4) δ 8.70 (dd, 1H), 8.55 − 8.48 (m, 1H), 7.65 (app t, 1H), 7.34 (dd, 1H), 7.24 (app t, 1H), 7.17 (d, 1H), 6.72 (app t, 1H), 6.56 (dd, 1H), 4.65 − 4.54 (m, 1H), 4.37 − 4.26 (m, 2H), 3.71 (s, 3H), 3.26 (dd, 1H), 3.15 (ddd, 1H), 3.04 − 2.89 (m, 2H). | 2.24 min, [MH]+ = 403 | 2 | 7-{1-[(3-methyloxetan-3- yl)methyl]-1H-pyrazol-4- yl}-5-{[(2S)-morpholin- 2-yl]methoxy}-1,6- naphthyridine |
| 13.10 | 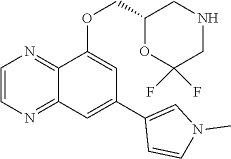 [SMILES: Cn1ccc(-c2cc(OC[C@@H]3CNCC(F)(F)O3)c3nccnc3c2)c1] | 1H NMR (400 MHz, Methanol- d4) δ 8.79 (d, 1H), 8.67 (d, 1H), 7.70 (d, 1H), 7.48 (d, 1H), 7.33 (t, 1H), 6.75 (dd, 1H), 6.60 (dd, 1H), 4.71 − 4.60 (m, 1H), 4.48 − 4.35 (m, 2H), 3.74 (s, 3H), 3.28 − 3.16 (m, 2H), 3.07 − 2.93 (m, 2H). | 2.10 min, [MH]+ = 404 | 2 | 7-(1-tert-butyl-1H-pyrrol- 3-yl)-5-{[(2S)-6,6- difluoromorpholin-2- yl]methoxy}-1,6- naphthyridine |
| 13.11 | 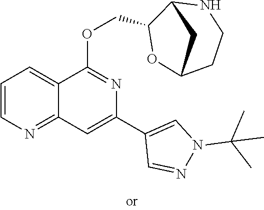 [SMILES: CC(C)(C)n1cc(-c2cc3ncccc3c(OC[C@H]3O[C@H]4CCN[C@@H]3C4)n2)cn1] | 1H NMR (400 MHz, Methanol- d4) δ 8.95 (dd, 1H), 8.65 (ddd, 1H), 8.42 (d, 1H), 8.16 (d, 1H), 7.66 (d, 1H), 7.52 (dd, 1H), 5.10 (dd, 1H), 5.00 (dd, 1H), 4.72 − 4.64 (m, 1H), 4.60 (ddd, 1H), 4.02 − 3.98 (m, 1H), 3.61 − 3.47 (m, 1H), 3.22 (dd, 1H), 2.37 − 2.25 (m, 1H), 2.08 (dd, 1H), 1.99 − 1.90 (m, 1H), 1.88 − 1.77 (m, 1H), 1.67 (s, 9H). | 1.72 min, [MH]+ = 394 | 2 | 7-(1-tert-butyl-1H- pyrazol-4-yl)-5- {[(1R,5S,7S)-6-oxa-2- azabicyclo[3.2.1]octan-7- yl]methoxy}-1,6- naphthyridine Or 7-(1-tert-butyl-1H-pyrazol- 4-yl)-5-{[(1S,5R, 7S)-6-oxa-2-azabicyclo[3. 2.1]octan-7-yl]methoxy}- 1,6-naphthyridine |
| | 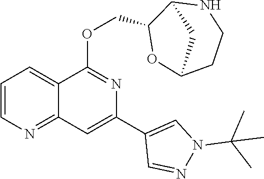 [SMILES: CC(C)(C)n1cc(-c2cc3ncccc3c(OC[C@H]3O[C@@H]4CCN[C@H]3C4)n2)cn1] | | | | |
| | (Absolute stereochemistry not determined) | | | | |
| 13.12 | 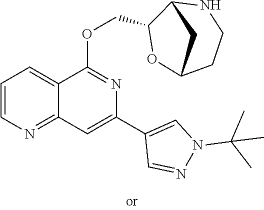 [SMILES: CC(C)(C)n1cc(-c2cc3ncccc3c(OC[C@H]3O[C@H]4CCN[C@@H]3C4)n2)cn1] | 1H NMR (400 MHz, Methanol- d4) δ 8.92 (dd, 1H), 8.63 (ddd, 1H), 8.37 (d, 1H), 8.13 (d, 1H), 7.62 (d, 1H), 7.50 (dd, 1H), 4.71 − 4.55 (m, 3H), 4.50 (dd, 1H), 3.68 (d, 1H), 3.17 (ddd, 1H), 2.91 (dd, 1H), 2.19 − 2.09 (m, 1H), 1.85 (dd, 1H), 1.74 − 1.62 (m, 2H), 1.66 (s, 9H). | 1.73 min, [MH]+ = 394 | 2 | 7-(1-tert-butyl-1H-pyrazol- 4-yl)-5-{[(1R,5S,7S)-6- oxa-2-azabicyclo[3.2.1] octan-7-yl]methoxy}- 1,6-naphthyridine Or 7-(1-tert-butyl-1H- pyrazol-4-yl)-5- {[(1S,5R, 7S)-6-oxa-2- azabicyclo[3.2.1]octan-7- yl]methoxy}-1,6- naphthyridine |
| | 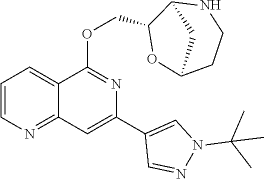 [SMILES: CC(C)(C)n1cc(-c2cc3ncccc3c(OC[C@H]3O[C@@H]4CCN[C@H]3C4)n2)cn1] | | | | |
| | (absolute stereochemistry not known) | | | | |
| 13.13 | 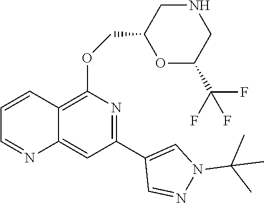 [SMILES: CC(C)(C)n1cc(-c2cc3ncccc3c(OC[C@@H]3CNC[C@H](C(F)(F)F)O3)n2)cn1] | 1H NMR (400 MHz, Methanol- d4) δ 8.92 (ddd, 1H), 8.61 − 8.56 (m, 1H), 8.40 (s, 1H), 8.15 (s, 1H), 7.62 (s, 1H), 7.53 − 7.46 (m, 1H), 5.17 (dd, 1H), 4.63 (dd, 1H), 4.45 − 4.31 (m, 2H), 3.17 − 3.06 (m, 2H), 3.03 − 2.90 (m, 2H), 1.66 (d, 9H) | 1.90 min, [MH]+ = 436 | 2 | 7-(1-tert-butyl-1H- pyrazol-4-yl)-5- ([(2S,6R)-6- (trifluoromethyl) morpholin-2-yl] methoxy}-1,6- naphthyridine |
| 13.14 | 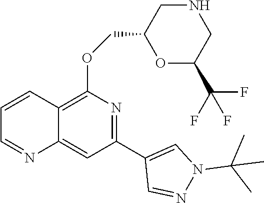 [SMILES: CC(C)(C)n1cc(-c2cc3ncccc3c(OC[C@@H]3CNC[C@@H](C(F)(F)F)O3)n2)cn1] | 1H NMR (400 MHz, Methanol-d4) δ 8.92 (dd, 1H), 8.60 − 8.55 (m, 1H), 8.37 (d, 1H), 8.12 (d, 1H), 7.62 (d, 1H), 7.50 (dd, 1H), 4.72 − 4.61 (m, 2H), 4.20 − 4.06 (m, 2H), 3.13 (ddd, 1H), 3.06 (dd, 1H), 2.83 − 2.73 (m, 2H), 1.66 (s, 9H) | 1.92 min, [MH]+ = 436 | 2 | 7-(1-tert-butyl-1H- pyrazol-4-yl)-5- {[(2S,6S)-6- (trifluoromethyl) morpholin-2-yl] methoxy}-1,6- naphthyridine |
| 13.15 | 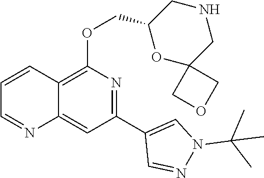 [SMILES: CC(C)(C)n1cc(-c2cc3ncccc3c(OC[C@@H]3CNCC4(COC4)O3)n2)cn1] | 1H NMR (400 MHz, Methanol- d4) δ 8.95 − 8.88 (m, 1H), 18.60 − 8.53 (m, 1H), 8.36 (d, 1H), 8.11 (d, 1H), 7.62 − 7.57 (m, 1H), 7.52 − 7.45 (m, 1H), 4.74 − 4.55 (m, 4H), 4.52 (d, 1H), 4.38 (d, 1H), 4.08 − 3.98 (m, 1H), 3.36 − 3.32 (m, 1H), 3.03 (ddd, 1H), 2.83 − 2.69 (m, 2H), 1.66 (s, 9H) | 1.74 min, [MH]+ = 410 | 2 | 7-(1-tert-butyl-1H- pyrazol-4-yl)-5-{[(6S)- 2,5-dioxa-8- azaspiro[3.5]nonan-6- yl]methoxy}-1,6- naphthyridine |
| 13.16 | 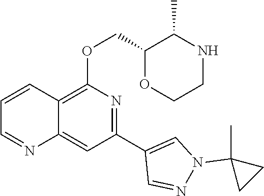 [SMILES: C[C@@H]1NCCO[C@@H]1COc1nc(-c2cnn(C3(C)CC3)c2)cc2ncccc12] | 1H NMR (400 MHz, Methanol- d4) δ 8.96 (dd, 1H), 8.59 (ddd, 1H), 8.39 (d, 1H), 8.12 (d, 1H), 7.66 (d, 1H), 7.53 (dd, 1H), 4.75 − 4.65 (m, 2H), 4.42 − 4.35 (m, 1H), 4.17 (dd, 1H), 3.92 − 3.79 (m, 2H), 3.45 (ddd, 1H), 3.11 (ddd, 1H), 1.67 (s, 3H), 1.53 (d, 3H), 1.38 − 1.26 (m, 2H), 1.06 − 1.00 (m, 2H) | 1.68 min, [MH]+ = 380 | 2 | 7-[1-(1- methylcyclopropyl)-1H- pyrazol-4-yl]-5- {[(2S,3S)-3- methylmorpholin-2- yl]methoxy}-1,6- naphthyridine |

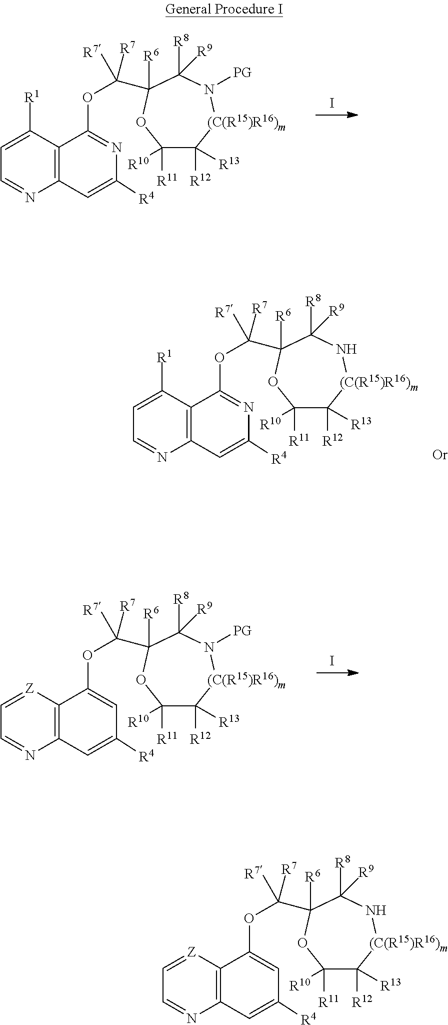

### Boc Deprotection

Conditions 1: To a solution of protected intermediate (1.0 eq) in dichloromethane (0.05-0.2 M) was added trifluoroacetic acid (6.0-60 eq). The reaction mixture was stirred at room temperature for 1-24 hours. On consumption of starting materials the reaction mixture was purified using one of the standard purification methods.

Conditions 2: A solution of intermediate (1.0 eq) in 1,4-dioxane/water (1:3 ratio, 0.05-0.2 M) was heated at 140-170° C. by microwave irradiation for 1-2 hours. The solvents were removed under reduced pressure and the reaction mixture was purified using one of the standard purification methods.

### Cbz Deprotection

To protected intermediate (1.0 eq) was added hydrobromic acid solution (30% wt, 25-50 eq) and the ensuing solution was stirred at 20° C. for 10 minutes. After this time, the reaction mixture was treated with hydrochloric acid (1 M aqueous solution) and the resulting mixture was extracted with dichloromethane. The aqueous phase was neutralised with aqueous sodium hydroxide solution and the products were extracted with ethyl acetate. The combined organic extracts were washed with brine, dried over anhydrous sodium/magnesium sulfate and concentrated to give the deprotected product.

TABLE 14
Analytical data for compounds synthesised by general method I:

| | | | LC MS | LC MS | |
|---|---|---|---|---|---|
| No | Structure | 1H NMR | RT, m/z | Method | Name |
| 14.1 | 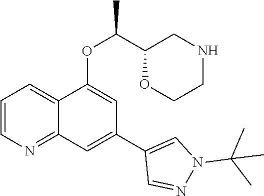 [SMILES: C[C@H](Oc1cc(-c2cnn(C(C)(C)C)c2)cc2ncccc12)[C@@H]1CNCCO1] | 1H NMR (400 MHz, Methanol- d4) δ 8.77 (dd, 1H), 8.62 (ddd, 1H), 8.35 (d, 1H), 8.03 (d, 1H), 7.76 − 7.72 (m, 1H), 7.43 − 7.38 (m, 1H), 7.33 (dd, 1H), 3.93 (dd, 1H), 3.78 (ddd, 1H), 3.66 (td, 1H), 2.99 (dd, 1H), 2.91 − 2.74 (m, 3H), 1.65 (s, 9H), 1.41 (d, 3H)- 1H not visible, hidden under the water peak. | 1.59 min, [MH]+ = 381 | 2 | 7-(1-tert-butyl-1H- pyrazol-4-yl)-5-[(1S)-1- [(2S)-morpholin-2- yl]ethoxy]quinoline |
| 14.2 | 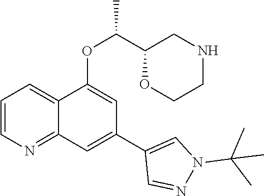 [SMILES: C[C@@H](Oc1cc(-c2cnn(C(C)(C)C)c2)cc2ncccc12)[C@@H]1CNCCO1] | 1H NMR (400 MHz, Methanol- d4) δ 8.77 (dd, 1H), 8.59 (dd, 1H), 8.35 (d, 1H), 8.03 (d, 1H), 7.74 (d, 1H), 7.41 (dd, 1H), 7.33 (d, 1H), 4.82 − 4.77 (m, 1H), 3.92 (d, 1H), 3.77 − 3.61 (m, 2H), 3.15 (d, 1H), 2.88 − 2.72 (m, 3H), 1.65 (s, 9H), 1.45 (d, 3H). | 1.60 min, [MH]+ = 381 | 2 | 7-(1-tert-butyl-1H- pyrazol-4-yl)-5-[(1R)-1- [(2S)-morpholin-2- yl]ethoxy]quinoline |
| 14.3 | 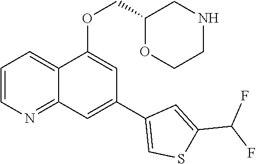 [SMILES: FC(F)c1cc(-c2cc(OC[C@@H]3CNCCO3)c3cccnc3c2)cs1] | 1H NMR (400 MHz, Methanol- d4) δ 8.83 (dd, 1H), 8.66 (ddd, 1H), 8.10 (app p, 1H), 7.92 (app dt, 1H), 7.85 (dd, 1H), 7.49 (dd, 1H), 7.34 (d, 1H), 7.09 (t, 1H), 4.34 − 4.24 (m, 2H), 4.02 (dtd, 1H), 3.97 − 3.91 (m, 1H), 3.72 (ddd, 1H), 3.09 (dd, 1H), 2.93 − 2.80 (m, 3H). | 1.70 min, [MH]+ = 377 | 2 | 7-[5- (difluoromethyl)thiophen- 3-yl]-5-{[(2S)-morpholin- 2-yl]methoxy}quinoline |
| 14.4 | 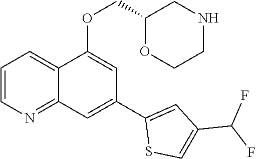 [SMILES: FC(F)c1csc(-c2cc(OC[C@@H]3CNCCO3)c3cccnc3c2)c1] | 1H NMR (400 MHz, Methanol- d4) δ 8.84 (dd, 1H), 8.66 (ddd, 1H), 7.84 (dd, 1H), 7.81 − 7.78 (m, 1H), 7.77 (s, 1H), 7.50 (dd, 1H), 7.33 (d, 1H), 6.83 (t, 1H), 4.34 − 4.24 (m, 2H), 4.06 − 3,98 (m, 1H), 3.97 − 3.90 (m, 1H), 3.72 (ddd, 1H), 3.08 (dd, 1H), 2.93 − 2.80 (m, 3H). | [MH]+ = 1.74 min, 377 | 2 | 7-[4- (difluoromethyl)thiophen- 2-yl]-5-{[(2S)-morpholin- 2-yl]methoxy}quinoline |
| 14.5 | 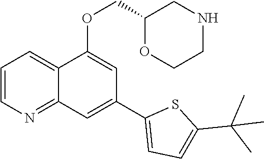 [SMILES: CC(C)(C)c1ccc(-c2cc(OC[C@@H]3CNCCO3)c3cccnc3c2)s1] | 1H NMR (400 MHz, Methanol- d4) δ 8.79 (dd, 1H), 8.62 (ddd, 1H), 7.75 (dd, 1H), 7.47 − 7.40 (m, 2H), 7.27 (d, 1H), 6.91 (d, 1H), 4.31 − 4.20 (m, 2H), 4.06 − 3.98 (m, 1H), 3.97 − 3.90 (m, 1H), 3.72 (td, 1H), 3.11 − 3.05 (m, 1H), 2.93 − 2.80 (m, 3H), 1.43 (s, 9H). | 1.97 min, [MH]+ = 383 | 2 | 7-(5-tert-butylthiophen-2- yl)-5-{[(2S)-morpholin-2- yl]methoxy}quinoline |
| 14.6 | 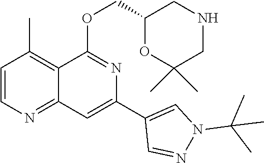 [SMILES: Cc1ccnc2cc(-c3cnn(C(C)(C)C)c3)nc(OC[C@@H]3CNCC(C)(C)O3)c12] | 1H NMR (400 MHz, Methanol- d4) δ 8.65 (d, 1H), 8.33 (d, 1H), 8.09 (d, 1H), 7.53 (s, 1H), 7.26 (dd, 1H), 4.58 − 4.42 (m, 2H), 4.26 (ddd, 1H), 3.05 (dd, 1H), 2.90 (s, 3H), 2.78 − 2.54 (m, 3H), 1.65 (s, 9H), 1.37 (s, 3H), 1.19 (s, 3H). | 1.78 min, [MH]+ = 410 | 2 | 7-(1-tert-butyl-1H- pyrazol-4-yl)-5-[(6,6- dimethylmorpholin-2- yl)methoxy]-4-methyl- 1,6-naphthyridine |
| 14.7 | 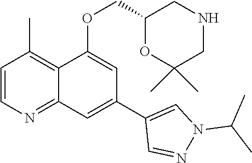 [SMILES: Cc1ccnc2cc(-c3cnn(C(C)C)c3)cc(OC[C@@H]3CNCC(C)(C)O3)c12] | 1H NMR (400 MHz, Methanol- d4) δ 8.54 (d, 1H), 8.27 (d, 1H) 8.01 (d, 1H), 7.70 (d, 1H), 7.21 − 7.13 (m, 2H), 4.59 (hept, 1H), 4.30 − 4.22 (m, 1H), 4.19 − 4.08 (m, 2H), 3.04 (dd, 1H), 2.96 (s, 3H), 2.78 − 2.58 (m, 3H), 1.56 (d, 6H), 1.38 (s, 3H), 1.20 (s, 3H). | 1.52 min, [MH]+ = 395 | 2 | 5-[(6,6- dimethylmorpholin-2- yl)methoxy]-4-methyl-7- [1-(propan-2-yl)-1H- pyrazol-4-yl]quinoline |
| 14.8 | 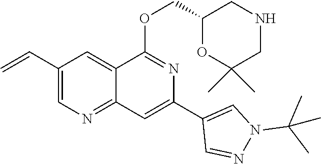 [SMILES: C=Cc1cnc2cc(-c3cnn(C(C)(C)C)c3)nc(OC[C@@H]3CNCC(C)(C)O3)c2c1] | 1H NMR (400 MHz, Methanol- d4) δ 9.11 (d, 1H), 8.57 − 8.50 (m, 1H), 8.39 (d, 1H), 8.14 (d, 1H), 7.66 (d, 1H), 6.96 (dd, 1H), 6.10 (d, 1H), 5.52 (d, 1H), 4.79 (dd, 1H), 4.68 (dd, 1H), 4.58 − 4.51 (m, 1H), 3.56 − 3.50 (m, 1H), 3.30 − 3.25 (m, 1H), 3.14 (dd, 1H), 3.02 (d, 1H), 1.66 (s, 9H), 1.47 (s, 3H), 1.37 (s, 3H). | 2.05 min, [MH]+ = 422 | 2 | 7-(1-tert-butyl-1H- pyrazol-4-yl)-5-[(6,6- dimethylmorpholin-2- yl)methoxy]-3-ethenyl- 1,6-naphthyridine |
| 14.9 | 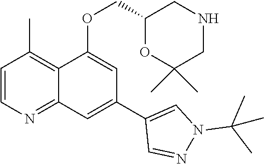 [SMILES: Cc1ccnc2cc(-c3cnn(C(C)(C)C)c3)cc(OC[C@@H]3CNCC(C)(C)O3)c12] | 1H NMR (600 MHz, Methanol- d4) δ 8.55 (d, 1H), 8.35 (d, 1H), 8.03 (d, 1H), 7.72 (d, 1H), 7.22 (d, 1H), 7.18 (dd, 1H), 4.29 − 4.24 (m, 1H), 4.18 (dd, 1H), 4.14 (dd, 1H), 3.04 (dd, 1H), 2.98 (s, 3H), 2.74 (d, 1H), 2.67 (dd, 1H), 2.61 (d, 1H), 1.66 (s, 9H), 1.39 (s, 3H), 1.20 (s, 3H). | 1.57 min, [MH]+ = 409 | | 7-(1-tert-butyl-1H- pyrazol-4-yl)-5-{[(2S)- 6,6-dimethylmorpholin-2- yl]methoxy}-4- methylquinoline |
| 14.10 | 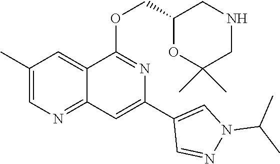 [SMILES: Cc1cnc2cc(-c3cnn(C(C)C)c3)nc(OC[C@@H]3CNCC(C)(C)O3)c2c1] | 1H NMR (600 MHz, Methanol- d4) δ 8.79 (d, 1H), 8.35 − 8.31 (m, 1H), 8.30 − 8.26 (m, 1H), 8.08 (d, 1H), 7.54 (d, 1H), 4.64 − 4.57 (m, 2H), 4.51 (dd, 1H), 4.36 − 4.29 (m, 1H), 3.20 (dd, 1H), 2.86 (dd, 1H), 2.77 − 2.69 (m, 2H), 2.52 (s, 3H), 1.56 (d, 6H), 1.40 (s, 3H), 1.25 (d, 3H). | 1.79 min, [MH]+ = 396 | 2 | 5-{[(2S)-6,6- dimethylmorpholin-2- yl]methoxy}-3-methyl-7- [1-(propan-2-yl)-1H- pyrazol-4-yl]-1,6- naphthyridine |
| 14.11 | 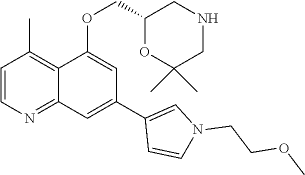 [SMILES: COCCn1ccc(-c2cc(OC[C@@H]3CNCC(C)(C)O3)c3c(C)ccnc3c2)c1] | 1H NMR (600 MHz, Methanol- d4) δ 8.49 (d, 1H), 7.64 (d, 1H), 7.33 (app t, 1H), 7.14 (d, 1H), 7.11 (dd, 1H), 6.84 − 6.79 (m, 1H), 6.57 (dd, 1H), 4.28 − 4.22 (m, 1H), 4.16 − 4.07 (m, 4H), 3.70 (t, 2H), 3.36 (s, 3H), 3.04 (dd, 1H), 2.95 (s, 3H), 2.73 (d, 1H), 2.66 (dd, 1H), 2.61 (d, 1H), 1.38 (s, 3H), 1.20 (s, 3H). | [MH]+ = 1.50 min, 410 | 2 | 5-{[(2S)-6,6- dimethylmorpholin-2- yl]methoxy}-7-[1-(2- methoxyethyl)-1H-pyrrol- 3-yl]-4-methylquinoline |
| 14.12 | 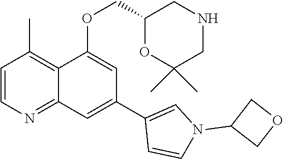 [SMILES: Cc1ccnc2cc(-c3ccn(C4COC4)c3)cc(OC[C@@H]3CNCC(C)(C)O3)c12] | 1H NMR (600 MHz, DMSO- d6) δ 8.56 (d, 1H), 7.88 (app t, 1H), 7.66 (d, 1H), 7.20 (d, 1H), 7.11 − 7.07 (m, 2H), 6.68 (dd, 1H), 5.43 − 5.35 (m, 1H), 4.95 (t, 2H), 4.86 (t, 2H), 4.12 − 4.07 (m, 2H), 4.05 (dd, 1H), 2.97 (dd, 1H), 2.85 (s, 3H), 2.61 (d, 1H), 2.49 2.42 (m, 2H), 1.30 (s, 3H), 1.09 (s, 3H). | 1.47 min, [MH]+ = 408 | 2 | 5-{[(2S)-6,6- dimethylmorpholin-2- yl]methoxy}-4-methyl-7- [1-(oxetan-3-yl)-1H- pyrrol-3-yl]quinoline |
| 14.13 | 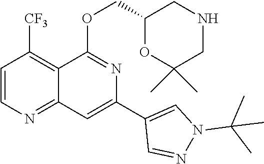 [SMILES: CC1(C)CNC[C@@H](COc2nc(-c3cnn(C(C)(C)C)c3)cc3nccc(C(F)(F)F)c23)O1] | 1H NMR (400 MHz, Methanol- d4) δ 9.09 (d, 1H), 8.41 (d, 1H), 8.15 (d, 1H), 7.84 (d, 1H), 7.78 (s, 1H), 4.68 (dd, 1H), 4.53 (dd, 1H), 4.09 − 4.01 (m, 1H), 3.96 − 3.89 (m, 1H), 3.69 (ddd, 1H), 3.11 (dd, 1H), 2.92 − 2.73 (m, 3H), 1.67 (s, 9H). | 1.93 min, [MH]+ = 436 | 2 | 7-(1-tert-butyl-1H- pyrazol-4-yl)-5-{[(2S)- 6,6-dimethylmorpholin-2- yl]methoxy}-4- (trifluoromethyl)-1,6- naphthyridine |
| 14.14 | 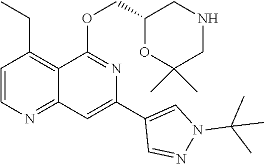 [SMILES: CCc1ccnc2cc(-c3cnn(C(C)(C)C)c3)nc(OC[C@@H]3CNCC(C)(C)O3)c12] | 1H NMR (400 MHz, Methanol- d4) δ 8.73 (d, 1H), 8.35 (d, 1H), 8.11 (d, 1H), 7.59 (s, 1H), 7.32 (d, 1H), 4.56 − 4.51 (m, 2H), 4.32 4.24 (m, 1H), 3.46 − 3.33 (m, 2H), 3.05 (dd, 1H), 2.76 − 2.59 (m, 3H), 1.66 (s, 9H), 1.41 − 1.35 (m, 6H), 1.21 (s, 3H) | 1.83 min, [MH]+ = 424 | 2 | 7-(1-tert-butyl-1H- pyrazol-4-yl)-5-{[(2S)- 6,6-dimethylmorpholin-2- yl]methoxy}-4-ethyl-1,6- naphthyridine |
| 14.15 | 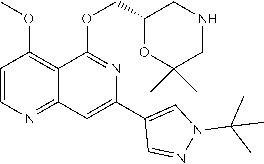 [SMILES: COc1ccnc2cc(-c3cnn(C(C)(C)C)c3)nc(OC[C@@H]3CNCC(C)(C)O3)c12] | 1H NMR (400 MHz, Methanol- d4) δ 8.67 (d, 1H), 8.33 (s, 1H), 8.09 (s, 1H), 7.49 (d, 1H), 6.99 (d, 1H), 4.56 (dd, 1H), 4.37 (dd, 1H), 4.27 − 4.19 (m, 1H), 4.08 (s, 3H), 3.14 (dd, 1H), 2.77 − 2.59 (m, 3H), 1.66 (s, 9H), 1.39 (s, 3H), 1.21 (s, 3H) | 1.57 min, [MH]+ = 426 | 2 | 7-(1-tert-butyl-1H- pyrazol-4-yl)-5-{[(2S)- 6,6-dimethylmorpholin-2- yl]methoxy}-4-methoxy- 1,6-naphthyridine |

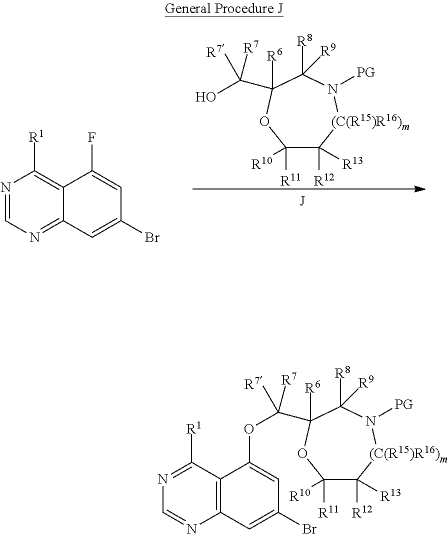

To a solution of alcohol (1.2 eq) in acetonitrile (0.05-0.1 M) cooled to 0° C. was added sodium hydride (57-63% oil dispersion, 2.0 eq) portion-wise and the reaction was stirred for 5 minutes. After this time, the appropriate heteroaryl chloride (1.0 eq) was added and the reaction was stirred at room temperature for 1-5 hours. The reaction mixture was quenched by slow addition of water and the products were extracted with ethyl acetate. The combined organics were washed with brine, dried over anhydrous sodium/magnesium sulfate and concentrated. The crude material was purified using purification method 1.

TABLE 15
Analytical data for compounds synthesised by general method J:

| | | | LC MS | LC MS |
|---|---|---|---|---|
| No | Structure | 1H NMR | RT, m/z | Method |
| 15.1 | 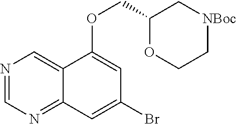 [SMILES: CC(C)(C)OC(=O)N1CCO[C@H](COc2cc(Br)cc3ncncc23)C1] | 1H NMR (400 MHz, Chloroform-d) δ 9.70 (s, 1H), 9.31 (s, 1H), 7.82 (dd, 1H), 7.06 (d, 1H), 4.25 (dd, 1H), 4.19 (dd, 1H), 4.17- 4.07 (m, 1H), 4.01-3.84 (m, 3H), 3.64 (td, 1H), 3.12-2.87 (m, 2H), 1.49 (s, 9H). | 2.86 min, [MH]+ = 424/426 | 2 |
| 15.2 | 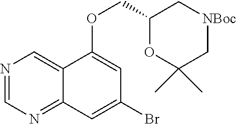 [SMILES: CC(C)(C)OC(=O)N1C[C@@H](COc2cc(Br)cc3ncncc23)OC(C)(C)C1] | No 1H NMR recorded | 3.16 min, [MH]+ = 452/454 | 2 |

### Synthesis of Key Building Blocks

### Synthesis of 1-[(dibenzylamino)methyl]cyclopropan-1-ol

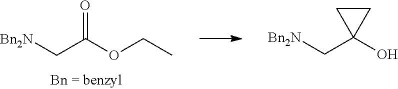

To a suspension of ethyl 2-(dibenzylamino)acetate (19.0 g, 67.1 mmol) in tetrahydrofuran (60 mL) cooled to 0° C. under nitrogen was added tetraisopropyl titanate (4.0 mL, 13.4 mmol) and bromo(ethyl)magnesium (2 M in tetrahydrofuran, 100 mL, 201.2 mmol) drop wise. The reaction was stirred at room temperature for 2 hours. The reaction mixture was quenched with saturated aqueous ammonium chloride solution and the products were extracted with ethyl acetate. The combined organics were dried over anhydrous sodium sulfate and concentrated. The crude material was purified by column chromatography on silica gel, eluting with 10-15% ethyl acetate in petroleum ether gradient to give 1-[(dibenzylamino)methyl]cyclopropan-1-ol (14.0 g, 52.4 mmol, 78% yield) as a colourless solid. LCMS (Method 5): Retention Time=1.44 minutes, [MH]+=268.

### Synthesis of 1-[(benzylamino)methyl]cyclopropan-1-ol

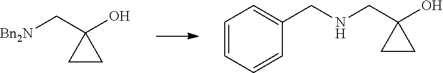

To a cooled suspension of 1-[(dibenzylamino)methyl]cyclopropan-1-ol (6.4 g, 23.9 mmol) in methanol (30 mL) and hydrochloric acid (6 N aqueous, 6.0 mL, 36.0 mmol) under nitrogen was added palladium(II) hydroxide (20% loading wet support, 640 mg, 0.46 mmol). A hydrogen bladder was attached, and the reaction was stirred at room temperature for 2 hours. The reaction mixture was filtered through Celite® using methanol and the filtrate was concentrated. Reaction was repeated at same scale, filtered and combined with the first reaction. The crude reaction mixture was basified with 28% aqueous ammonia solution and the products were extracted with diethyl ether. The combined organic layers were dried over anhydrous sodium sulfate and concentrated to give 1-[(benzylamino)methyl]cyclopropan-1-ol (8.0 g, 45.1 mmol, 94% yield) as a light yellow liquid. No purification was carried out and the product was used crude in the next step. LCMS (Method 5): Retention Time==0.92 minutes, [MH]+=178.

### Synthesis of [(5S)-7-benzyl-4-oxa-7-azaspiro[2.5]octan-5-yl]methanol

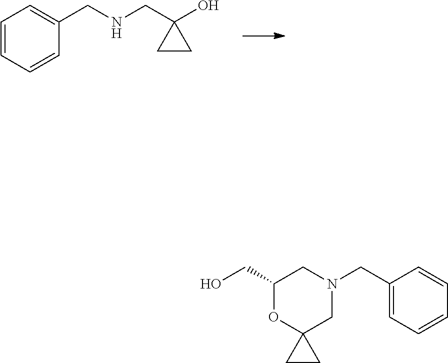 [SMILES: OC1(CNCc2ccccc2)CC1 ; OC[C@@H]1CN(Cc2ccccc2)CC2(CC2)O1]

To a suspension of 1-[(benzylamino)methyl]cyclopropan-1-ol (8.0 g, 45.1 mmol) in toluene (30 mL) was added (R)-(−)-epichlorohydrin (5.4 mL, 69.2 mmol) and lithium perchlorate (4.8 g, 45.1 mmol). The reaction was stirred at room temperature for 72 hours. Sodium methoxide (6.1 g, 112.8 mmol) was then added and the reaction was stirred at room temperature for a further 16 hours. The reaction mixture was quenched with saturated aqueous ammonium chloride solution and the products were extracted with ethyl acetate. The combined organic layers were dried over anhydrous sodium sulfate and concentrated. The crude material was purified by column chromatography on silica gel eluting with 40-50% ethyl acetate in petroleum ether gradient to give [(5S)-7-benzyl-4-oxa-7-azaspiro[2.5]octan-5-yl]methanol (4.2 g, 18.0 mmol, 40% yield) as a light brown gum. 1H NMR (400 MHz, DMSO-d6) δ 7.41-7.18 (m, 5H), 4.60 (t, 1H), 3.63-3.53 (m, 1H), 3.47 (s, 2H), 3.38-3.34 (m, 1H), 330-3.21 (m, 1H), 2.82 (dt, 1H), 2.12 (dd, 1H), 1.92-1.79 (m, 1H), 0.76-0.64 (m, 1H), 0.64-0.53 (m, 1H), 0.53-0.44 (m, 1H), 0.44-0.35 (m, 1H). LCMS (Method 5): Retention Time=1.18 minutes, [MH]+=234.

### Synthesis of [(5S)-4-oxa-7-azaspiro[2.5]octan-5-yl]methanol

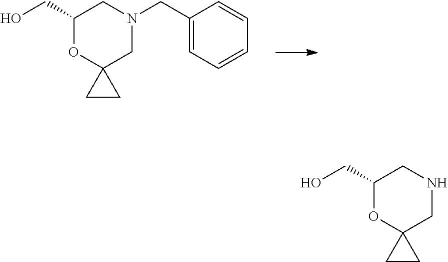 [SMILES: OC[C@@H]1CN(Cc2ccccc2)CC2(CC2)O1 ; OC[C@@H]1CNCC2(CC2)O1]

To a suspension of [(5S)-7-benzyl-4-oxa-7-azaspiro[2.5]octan-5-yl]methanol (4.2 g, 18.0 mmol) in methanol (30 mL) under nitrogen was added palladium(II) hydroxide (20% loading wet support, 400 mg, 0.29 mmol), a hydrogen bladder was attached and the reaction was stirred at room temperature for 2 hours. The reaction mixture was filtered through Celite® washing with methanol and the filtrate was concentrated to give [(5S)-4-oxa-7-azaspiro[2.5]octan-5-yl]methanol (2.3 g, 16.1 mmol, 89% yield). 1H NMR (400 MHz, Methanol-d4) 3.69-3.61 (m, 1H), 3.45 (dd, 2H), 3.19 (dd, 1H), 2.91 (ddd, 1H), 2.57 (dd, 1H), 2.24 (dd, 1H), 0.85-0.78 (m, 1H), 0.73-0.66 (m, 1H), 0.59-0.50 (m, 2H). LCMS (Method 5): Retention Time=0.52 minutes, [MH]+=144.

### Synthesis of tert-butyl (5S)-5-(hydroxymethyl)-4-oxa-7-azaspiro[2.5]octane-7-carboxylate

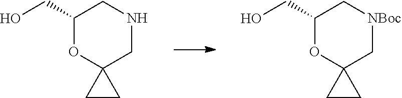 [SMILES: OC[C@@H]1CNCC2(CC2)O1 ; CC(C)(C)OC(=O)N1C[C@@H](CO)OC2(CC2)C1]

To a solution of [(5S)-4-oxa-7-azaspiro[2.5]octan-5-yl]methanol (1.4 g, 9.8 mmol) in dichloromethane (10 mL) and water (20 mL) was added sodium hydroxide (390 mg, 9.8 mmol). A solution of di-tert-butyl dicarbonate (2.1 g, 9.8 mmol) in dichloromethane (10 mL) was added dropwise and the reaction was stirred at room temperature for 2-3 hours. The reaction mixture was poured onto ice-cold water and the products were extracted with dichloromethane. The combined organic layers were washed with brine, dried over anhydrous sodium sulfate and concentrated. The crude material was purified by column chromatography on silica gel eluting with 10-15% ethyl acetate in petroleum ether gradient to give tert-butyl (5S)-5-(hydroxymethyl)-4-oxa-7-azaspiro[2.5]octane-7-carboxylate (1.1 g, 4.5 mmol, 46% yield) as a syrupy liquid. 1H NMR (400 MHz, DMSO-d6) δ 4.72 (t, 1H), 3.93 (br s, 1H), 3.55-3.42 (m, 1H) 3.42-3.17 (m, 4H), 2.67 (br s, 1H), 1.40 (s, 9H), 0.81-0.73 (m, 1H), 0.72-0.61 (m, 1H), 0.61-0.51 (m, 1H), 049-0.39 (m, 1H). LCMS (Method 5): Retention Time=2.40 minutes, [(M−100)H]+=144.

### Synthesis of Synthesis of tert-butyl (2S)-2-(1-hydroxyethyl)morpholine-4-carboxylate

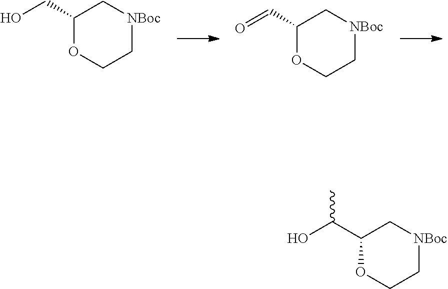 [SMILES: CC(C)(C)OC(=O)N1CCO[C@H](CO)C1 ; CC(C)(C)OC(=O)N1CCO[C@H](C=O)C1 ; CC(O)[C@@H]1CN(C(=O)OC(C)(C)C)CCO1]

To a solution of (S)—N-boc-2-hydroxymethylmorpholine (3.4 g, 15.7 mmol) in dichloromethane (30 mL) cooled to 0° C. was added Dess-Martin periodinane (8.0 g, 18.8 mmol) and the reaction was stirred at room temperature for 16 hours. The reaction mixture was quenched with saturated aqueous sodium bicarbonate solution and diluted with dichloromethane. The resulting precipitate was removed by filtration. The products were extracted with dichloromethane. The combined organic layers were washed with brine, dried over anhydrous sodium sulfate and concentrated. The residue was purified by column chromatography on silica gel eluting with 5-80% tert-butyl methyl ether in heptane gradient to give tert-butyl (2S)-2-formylmorpholine-4-carboxylate (2.2 g, 7.5 mmol, 48% yield) as colourless residue.

To a solution of tert-butyl (2S)-2-formylmorpholine-4-carboxylate (2.1 g, 9.8 mmol) in tetrahydrofuran (30 mL) cooled to 0° C. was added methylmagnesium chloride (3 M in tetrahydrofuran, 3.9 mL, 11.7 mmol) and the reaction was stirred at room temperature for 16 hours. The crude reaction mixture was quenched with saturated aqueous ammonium chloride solution and the products were extracted with ethyl acetate. The combined organics layers were washed with brine, dried over anhydrous magnesium sulfate and concentrated. The crude material was purified by column chromatography on silica gel eluting with 5-60% tert-butyl methyl ether in heptane gradient to give tert-butyl (2S)-2-(1-hydroxyethyl)morpholine-4-carboxylate (999 mg, 4.1 mmol, 42% yield) as a mixture of diastereoisomers in a 3:2 ratio. 1H NMR (400 MHz, Chloroform-d) δ 4.04-3.77 (m, 3H), 3.71-3.61 (m, 0.5H), 3.59-3.47 (m, 1.5H), 3.34-3.24 (m, 0.5H), 3.17 (ddd, 0.5H), 3.00-2.87 (n, 1.5H), 282 (dd, 0.5H), 2.77-2.57 (m, 0.5H), 2.52-2.33 (m, 0.5H), 1.47 (s, 9H), 1.20 (d, 1.5H), 1.18 (d, 1.5H). Mixture of diastereoisomers in a 3:2 ratio but signals assigned as 1:1 ratio.

### Synthesis of 1-(benzylamino)-2-methylpropan-2-ol

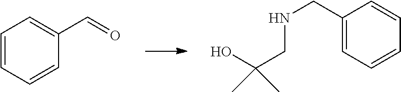

A suspension of benzaldehyde (10.0 g, 94.2 mmol) and 1-amino-2-methylpropan-2-ol (8.4 g, 94.2 mmol) in ethanol (40 mL) was stirred at room temperature for 3 hours. The reaction was cooled to 0° C. and sodium borohydride (3.9 g, 103.7 mmol) was added portion wise. The reaction was stirred at room temperature overnight. The reaction mixture was quenched with ice cold water and the products were extracted with ethyl acetate. The combined organic layers were dried over anhydrous sodium sulfate and concentrated to give 1-(benzylamino)-2-methylpropan-2-ol (14.6 g, 81.4 mmol, 86% yield) as a colourless solid. 1H NMR (400 MHz, DMSO-d6) δ 7.36-7.27 (m, 4H), 7.26-7.17 (m, 1H), 4.20 (br s, 1H), 3.72 (s, 2H), 2.36 (s, 2H), 1.09 (s, 6H). LCMS (Method 5): Retention Time=1.46 minutes, [MH]+=180.

### Synthesis of [(2S)-4-benzyl-6,6-dimethylmorpholin-2-yl]methanol

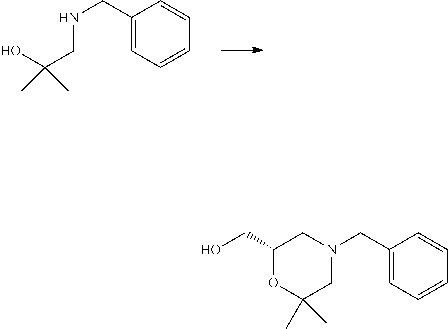 [SMILES: CC(C)(O)CNCc1ccccc1 ; CC1(C)CN(Cc2ccccc2)C[C@@H](CO)O1]

To a solution of 1-(benzylamino)-2-methylpropan-2-ol (12.0 g, 66.9 mmol) in toluene (200 mL) was added ((R)-(−)-epichlorohydrin (9.3 mg, 100.4 mmol) followed by the slow addition of lithium perchlorate (7.1 g, 66.9 mmol). The reaction was stirred at room temperature for 72 hours. Sodium methoxide (25% w/w methanolic solution, 14.2 mL, 66.9 mmol) was added and the reaction was stirred at room temperature for 16 hours. The reaction mixture was quenched with water and the products were extracted with ethyl acetate. The combined organic layers were washed with brine, dried over anhydrous sodium sulfate and concentrated. The crude material was purified by column chromatography on silica gel eluting with 30-50% ethyl acetate in petroleum ether gradient to give [(2S)-4-benzyl-6,6-dimethylmorpholin-2-yl]methanol (8.0 g, 34.0 mmol, 51% yield) as syrupy liquid. 1H NMR (400 MHz, DMSO-d6): δ 7.33-7.25 (m, 51H), 4.58-4.55 (m, 1H), 3.70-3.68 (m, 1H), 3.45-3.33 (m, 3H), 3.20-3.18 (m, 1H), 2.81-2.79 (m, 1H), 2.51-2.47 (m, 1H), 1.75-1.72 (m, 1H), 1.65-1.59 (m, 1H), 1.27 (s, 3H), 1.05 (s, 3H). LCMS (Method 5): Retention Time=0.99 minutes, [MH]+=236.

### Synthesis of [(2S)-6,6-dimethylmorpholin-2-yl]methanol

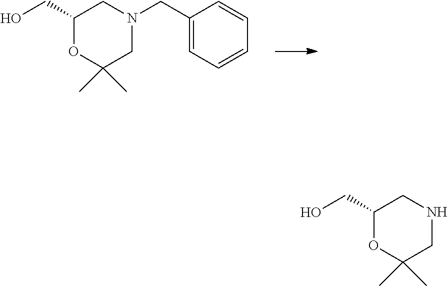

To a solution of [(2S)-4-benzyl-6,6-dimethylmorpholin-2-yl]methanol (13.5 g, 57.4 mmol) in methanol (200 mL) under nitrogen was added palladium(II) hydroxide (20% loading wet support, 1.4 g, 1.0 mmol), a hydrogen bladder was attached and the reaction was stirred at room temperature for 2-3 hours. The reaction mixture was filtered through Celite® eluting with methanol and the filtrate was concentrated to give [(2S)-6,6-dimethylmorpholin-2-yl]methanol (8.3 g, 57.2 mmol, 100% yield). No purification was carried out and the product was used crude in the next step. 1H NMR (400 MHz, DMSO-d6): δ 4.49 (br s 1H), 3.55-3.52 (m, 1H), 3.29-3.14 (m, 2H), 2.83-2.80 (m, 1H), 2.51-2.50 (m, 1H), 2.35-2.17 (m, 1H), 1.20 (s, 3H), 1.00 (s, 3H). LCMS (Method 5): Retention Time=128 minutes, [MH]+=146.

### Synthesis of tert-butyl (6S)-6-(hydroxymethyl)-2,2-dimethylmorpholine-4-carboxylate

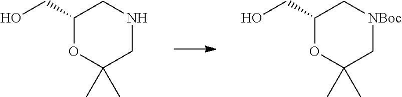

To a solution of [(2S)-6,6-dimethylmorpholin-2-yl]methanol (8.3 g, 57.2 mmol) in a mixture of dichloromethane (100 mL) and water (50 mL) was added di-tert-butyl dicarbonate (12.5 g, 57.2 mmol) and aqueous sodium hydroxide solution (2 N, 28.6 mL, 57.2 mmol). The reaction was stirred at room temperature for 4-5 hours. The reaction mixture was quenched with ice cold water and the products were extracted with dichloromethane. The combined organics layers were washed with brine, dried over anhydrous sodium sulfate and concentrated. The crude material was purified by column chromatography on silica gel eluting with 10-20% ethyl acetate in petroleum ether gradient to give tert-butyl (6S)-6-(hydroxymethyl)-2,2-dimethylmorpholine-4-carboxylate (11.2 g, 45.7 mmol, 80% yield) as syrupy liquid. 1H NMR (400 MHz, DMSO-d6) δ 4.68 (t, 1H), 4.04-3.80 (m, 1H), 3.73-3.52 (m, 2H), 3.37 (ddd, 1H), 3.29-3.20 (m, 1H), 2.75-2.56 (m, 1H), 2.48-2.35 (m, 1H), 1.40 (s, 9H), 1.11 (s, 6H). LCMS (Method 5): Retention Time=1.80 minutes, [(M−56)H]+=190.

### Synthesis of (2S)-1-amino-3-(benzyloxy)propan-2-ol

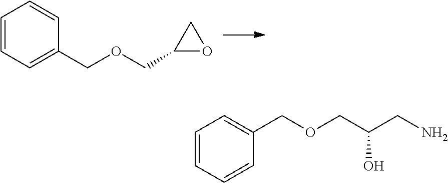 [SMILES: c1ccc(COC[C@@H]2CO2)cc1 ; NC[C@H](O)COCc1ccccc1]

A solution of (2S)-2-[(benzyloxy)methyl]oxirane (10.0 g 60.9 mmol) in ammonium hydroxide (25-30% in water, 100 mL) was stirred at room temperature for 16 hours. The volatiles were removed under reduced pressure and the resulting mixture was azeotroped with toluene to give (2S)-1-amino-3-(benzyloxy)propan-2-ol (11.1 g, 61.2 mmol, 100% yield). 1H NMR (400 MHz, Deuterium Oxide) δ 7.47-7.30 (m, 5H), 4.62-4.51 (m, 2H), 3.83-3.73 (m, 1H), 3.56 (dd, 1H), 3.52-3.42 (m, 1H), 2.74-2.65 (m, 1H), 2.64-2.54 (m, 1H). LCMS (Method 4-Column 7): Retention Time=1.14 minutes, [MH]+=182.

### Synthesis of (2R)—N-[(2S)-3-(benzyloxy)-2-hydroxypropyl]-2-chloropropanamide

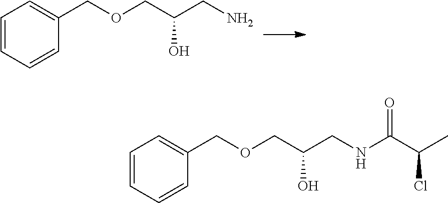 [SMILES: NC[C@H](O)COCc1ccccc1 ; C[C@@H](Cl)C(=O)NC[C@H](O)COCc1ccccc1]

To a solution of (2S)-1-amino-3-phenylmethoxypropan-2-ol (11.0 g, 60.7 mmol) in ethanol (90 mL) was added methyl (2R)-2-chloropropanoate (8.2 g, 66.8 mmol) dropwise. The reaction was stirred at 80° C. for 28 hours. The solvents were removed under reduced pressure and the residue was diluted with ethyl acetate. The organic layers were washed with 1 N hydrochloric acid solution and brine, dried over anhydrous sodium sulfate and concentrated. The crude material was purified by column chromatography on silica gel eluting with 25% ethyl acetate in hexane to give (2R)—N-[(2S)-3-(benzyloxy)-2-hydroxypropyl]-2-chloropropanamide (6.2 g, 22.8 mmol, 38% yield) as a pale yellow oil. 1H NMR (400 MHz, DMSO-d6) δ 8.26-8.12 (m, 1H), 7.44-7.19 (m, 5H), 5.04 (d, 1H), 4.60-4.50 (m, 1H), 4.48 (s, 2H), 3.75-3.63 (m, 1H), 3.44-3.36 (m, 1H), 3.31-3.20 (m, 1H), 3.09-2.93 (m, 1H), 1.49 (d, 3H). LCMS (Method 8-Column 2): Retention Time=7.83 minutes, [MH]+=272.

### Synthesis of (2S,6S)-6-[(benzyloxy)methyl]-2-methylmorpholin-3-one

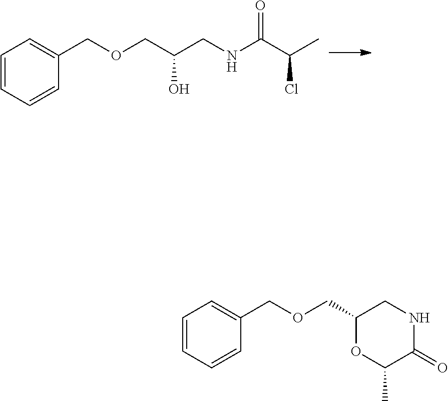 [SMILES: C[C@@H](Cl)C(=O)NC[C@H](O)COCc1ccccc1 ; C[C@@H]1O[C@H](COCc2ccccc2)CNC1=O]

To a solution of (2R)-2-chloro-N-[(2S)-2-hydroxy-3-phenylmethoxypropyl]propanamide (6.2 g, 22.8 mmol) in tetrahydrofuran (310 mL) cooled to 0° C. was added sodium hydride (57-63% w/w oil dispersion, 3.7 g, 91.3 mmol). The reaction was stirred at room temperature for 3 hours. The reaction was quenched with isopropyl alcohol (14 mL) acidified by the addition of Dowex® 50H⁺ resin (1.5 g) and filtered. The filtrate was washed with water and the products were extracted with ethyl acetate. The combined organic layers were dried over anhydrous sodium sulfate and concentrated. The crude material was purified by column chromatography on silica gel eluting with 2% methanol in dichloromethane to give (2S,6S)-6-[(benzyloxy)methyl]-2-methylmorpholin-3-one (3.9 g, 16.6 mmol, 73% yield) as a pale yellow oil. 1H NMR (400 MHz, DMSO-d6) δ 7.85 (d, 1H), 7.40-7.24 (m, 5H), 4.55-4.48 (m, 2H), 4.09 (q, 1H), 3.98-3.86 (m, 1H), 3.55-3.43 (m, 2H), 3.21-3.06 (m, 2H), 1.25 (d, 3H). LCMS (Method 8-Column 2): Retention Time=7.75 minutes, [MH]+=236.

### Synthesis of (2S,6S)-2-[(benzyloxy)methyl]-6-methylmorpholine

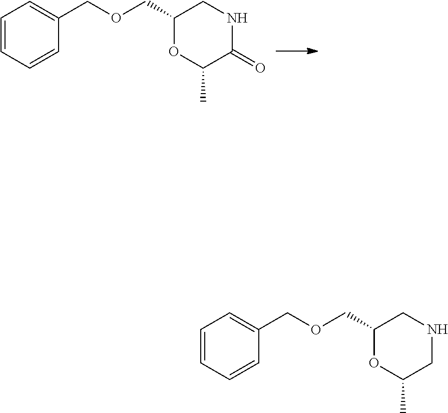 [SMILES: C[C@@H]1O[C@H](COCc2ccccc2)CNC1=O ; C[C@H]1CNC[C@@H](COCc2ccccc2)O1]

To a solution of (2S,6S)-2-methyl-6-(phenylmethoxymethyl)morpholin-3-one (3.8 g, 16.1 mmol) in tetrahydrofuran (30 mL) cooled to 0° C. under nitrogen was added lithium aluminium hydride (1 M solution in tetrahydrofuran, 48.5 mL, 48.5 mmol). The reaction was stirred at room temperature for 16 hours. The reaction mixture was quenched with saturated aqueous ammonium chloride solution and the products were extracted with dichloromethane. The combined organic layers were dried over anhydrous sodium sulfate and concentrated under reduced pressure. The crude material was purified by column chromatography on silica gel eluting with 8% methanol in dichloromethane to give (2S,6S)-2-[(benzyloxy)methyl]-6-methylmorpholine (2.8 g, 12.7 mmol, 78% yield) as a yellow oil. 1H NMR (400 MHz, DMSO-d6) δ 7.40-723 (m, 5H), 4.48 (s, 2H), 3.84-3.75 (m, 1H), 3.74-363 (m, 1H), 3.45 (dd, 1H), 3.40 (dd, 1H), 3.05-2.94 (m, 2H), 2.64-2.52 (m, 1H), 2.42 (dd, 1H), 1.07 (d, 3H). LCMS (Method 8-Column 2): Retention Time=10.50 minutes, [MH]+=222.

### Synthesis of tert-butyl (2S,6S)-2-[(benzyloxy)methyl]-6-methylmorpholine-4-carboxylate

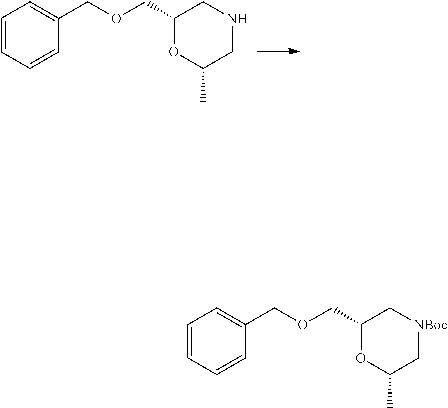 [SMILES: C[C@H]1CNC[C@@H](COCc2ccccc2)O1 ; C[C@H]1CN(C(=O)OC(C)(C)C)C[C@@H](COCc2ccccc2)O1]

To a solution of (2S,6S)-2-methyl-6-(phenylmethoxynethyl)morpholine (2.8 g, 12.7 mmol) in dichloromethane (50 mL) was added di-tert-butyl dicarbonate (6.8 g, 31.6 mmol), 4-(dimethylamino)pyridine (0.1 g, 0.82 mmol) and triethylamine (4.2 mL, 31.6 mmol). The reaction was stirred at room temperature for 3 hours. The reaction mixture was poured onto water and the products were extracted with dichloromethane. The combined organic layers were dried over anhydrous sodium sulfate and concentrated. The crude material was purified by column chromatography on silica gel eluting with 8% ethyl acetate in hexane to give tert-butyl (2S,6S)-2-[(benzyloxy)methyl]-6-methylmorpholine-4-carboxylate (3.2 g, 9.6 mmol, 76% yield) as a light yellow oil. 1H NMR (400 MHz, DMSO-d6) δ 7.42-7.24 (m, 5H), 4.49 (s, 2H), 3.94-3.67 (m, 2H), 3.62-3.51 (m, 1H), 3.51-3.38 (m, 3H), 1.40 (s, 9H), 1.07 (d, 3H). LCMS (Method 8-Column 2): Retention Time=9.94 minutes, [MH]+=266.

### Synthesis of tert-butyl (2S,6S)-2-(hydroxymethyl)-6-methylmorpholine-4-carboxylate

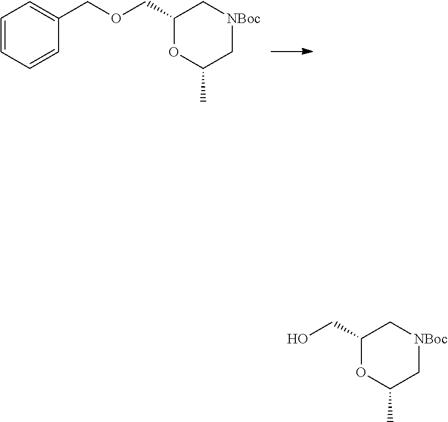 [SMILES: C[C@H]1CN(C(=O)OC(C)(C)C)C[C@@H](COCc2ccccc2)O1 ; C[C@H]1CN(C(=O)OC(C)(C)C)C[C@@H](CO)O1]

To a solution of (tert-butyl (2S,6S)-2-[(benzyloxy)methyl]-6-methylmorpholine-4-carboxylate (2.8 g, 8.7 mmol) in methanol (30 mL) was added palladium(II) hydroxide (20% loading wet support, 1.0 g, 0.71 mmol) and the reaction was stirred at room temperature for 1.5 hours. The reaction mixture was filtered through Celite®, and the filtrate was concentrated under reduced pressure. The crude material was purified by column chromatography on silica gel eluting with 15-25% ethyl acetate in hexane gradient to give tert-butyl (2S,6S)-2-(hydroxymethyl)-6-methylmorpholine-4-carboxylate (1.4 g, 6.1 mmol, 70% yield) as a yellow liquid. 11H NMR (400 MHz, Chloroform-d) δ 4.03-3.75 (m, 2H), 3.73-3.64 (m, 1H), 3.62-3.50 (m, 3H), 2.80-2.39 (m, 2H), 1.46 (s, 9-H), 1.19 (d, 3H). LCMS (Method 4-Column 1): Retention Time=1.33 minutes, [(M−56)H]+=177.

### Synthesis of (2S)—N-[(2S)-3-(benzyloxy)-2-hydroxypropyl]-2-chloropropanamide

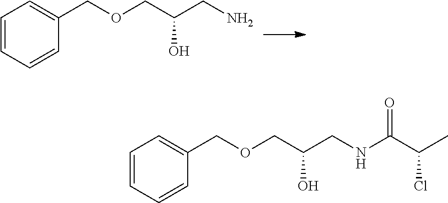 [SMILES: NC[C@H](O)COCc1ccccc1 ; C[C@H](Cl)C(=O)NC[C@H](O)COCc1ccccc1]

To a solution of (2S)-1-amino-3-phenylmethoxypropan-2-ol (10.5 g, 57.9 mmol) in ethanol (34 mL) was added methyl (2S)-2-chloropropanoate (7.8 g, 63.7 mmol) dropwise over 15 minutes. The reaction was heated to 75° C. for 16 hours. The solvents were removed under reduced pressure and the resulting residue was diluted with ethyl acetate. The organic layer was washed with 1 N hydrochloric acid solution and brine, dried over anhydrous sodium sulfate and concentrated. The crude material was purified by column chromatography on silica gel eluting with 2-5% methanol in dichloromethane gradient to give (2S)—N-[(2S)-3-(benzyloxy)-2-hydroxypropyl]-2-chloropropanamide (8.0 g, 25.9 mmol, 45% yield) as a yellow liquid. 1H NMR (400 MHz, DMSO-d6) δ 8.27-8.14 (m, 1H), 7.40-7.24 (m, 5H), 5.03 (d, 1H), 4.53 (q, 1H), 4.50-4.45 (m, 2H), 3.77-3.63 (m, 1H), 3.29-3.18 (m, 1H), 3.10-2.97 (m, 1H), 1.49 (d, 3H). LCMS (Method 4-Column 1): Retention Time=1.38 minutes, [MH]+=272.

### Synthesis of (2R,6S)-6-[(benzyloxy)methyl]-2-methylmorphlin-3-one

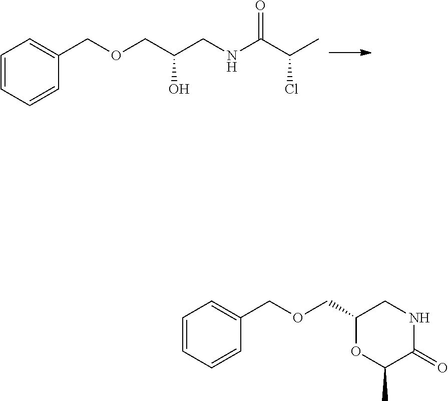 [SMILES: C[C@H](Cl)C(=O)NC[C@H](O)COCc1ccccc1 ; C[C@H]1O[C@H](COCc2ccccc2)CNC1=O]

To a solution of (2S)-2-chloro-N-[(2S)-2-hydroxy-3-phenylmethoxypropyl]propanamide (8.0 g, 29.4 mmol) in tetrahydrofuran (200 mL) cooled to 0° C. was added portion-wise sodium hydride (57-63% w/w oil dispersion, 4.7 g, 117.8 mmol). The reaction was stirred at 0° C. for 5 minutes then at room temperature for 6 hours. The reaction mixture was quenched with isopropyl alcohol (30 mL) and acidified by portion-wise addition of Dowex® 50H⁺ resin (50 g). The reaction was filtered, and the resin was washed well with ethyl acetate. The filtrate was then concentrated and the crude material was purified by column chromatography on silica gel eluting with 2-5% methanol in dichloromethane to give (2R,6S)-6-[(benzyloxy)methyl]-2-methylmorpholin-3-one (5.5 g, 23.4 mmol, 79% yield). 1H NMR (400 MHz, DMSO-d6) δ 7.91 (s, 1H), 7.42-7.21 (m, 5H), 4.51 (s, 2H), 4.19 (q, 1H), 4.03 (dq, 1H), 3.58-3.44 (m, 2H), 3.25-3.03 (m, 2H), 1.29 (d, 3H). LCMS (Method 4-Column 1): Retention Time=no UV signal, [MH]+=236.

### Synthesis of (2S,6R)-2-[(benzyloxy)methyl]-6-methylmorpholine

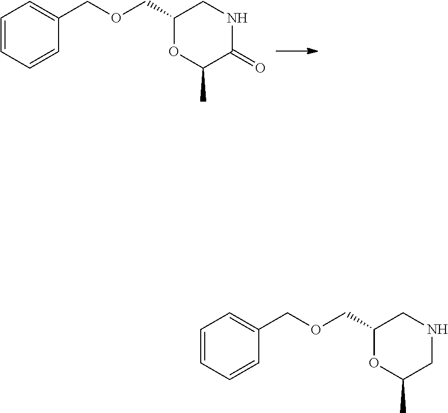 [SMILES: C[C@H]1O[C@H](COCc2ccccc2)CNC1=O ; C[C@@H]1CNC[C@@H](COCc2ccccc2)O1]

To a solution of (2R,6S)-2-methyl-6-(phenylmethoxymethyl)morpholin-3-one (5.5 g, 23.4 mmol) in tetrahydrofuran (30 mL) cooled to 0° C. under nitrogen was added lithium aluminium hydride (1 M solution in tetrahydrofuran, 25.7 mL, 25.7 mmol). The reaction was stirred at room temperature for 2.5 hours. The reaction was cooled to 0° C. and quenched with water followed by sodium hydroxide solution (1 N aqueous, 1 mL). The reaction was then filtered through Celite®, washing well with dichloromethane. The combined organics were washed with water, dried over anhydrous sodium sulfate and concentrated. The crude material was purified by column chromatography on silica gel eluting with 2-5% methanol in dichloromethane gradient to give (2S,6R)-2-[(benzyloxy)methyl]-6-methylmorpholine (3.6 g, 16.3 mmol, 70% yield) as a yellow liquid. 1H NMR (400 MHz, DMSO-d6) δ 7.41-7.23 (m, 5H), 4.58-4.41 (m, 2H), 3.91-3.73 (m, 2H), 3.65-3.53 (m, 2H), 2.87-2.78 (m, 2H), 2.67 (dd, 1H), 2.44 (dd, 1H), 1.09 (d, 3H). LCMS (Method 4-Column 7): Retention Time=0.95 minutes, [MH]+=222.

### Synthesis of tert-butyl (2S,6R)-2-[(benzyloxy)methyl]-6-methylmorpholine-4-carboxylate

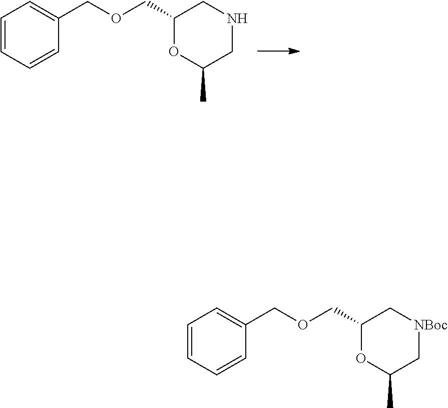 [SMILES: C[C@@H]1CNC[C@@H](COCc2ccccc2)O1 ; C[C@@H]1CN(C(=O)OC(C)(C)C)C[C@@H](COCc2ccccc2)O1]

To a solution of (2S,6R)-2-methyl-6-(phenylmethoxymethyl)morpholine (3.6 g, 16.3 mmol) in dichloromethane (25 mL) were added di-tert-butyl dicarbonate (9.2 g, 42.3 mmol), 4-(dimethylamino)pyridine (120 mg, 0.98 mmol) and triethylamine (4.3 mL, 42.3 mmol). The reaction was stirred at room temperature for 3 hours. The reaction was poured onto water and the products were extracted with ethyl acetate. The combined organics were dried over anhydrous sodium sulfate and concentrated. The crude material was purified by column chromatography on silica gel eluting with 6-8% ethyl acetate in hexane to give tert-butyl (2S,6R)-2-[(benzyloxy)methyl]-6-methylmorpholine-4-carboxylate (4.0 g, 12.1 mmol, 74% yield) as a light yellow oil. 1H NMR (400 MHz, DMSO-d6) δ 7.42-7.24 (m, 5H), 4.50 (s, 2H), 3.95-3.71 (m, 2H), 3.61-3.18 (m, 5H), 3.12-2.74 (m, 1H), 1.39 (s, 9H), 1.07 (d, 3H). LCMS (Method 4-Column 2): Retention Time=2.20 minutes, [(M−56)H]+=266.

### Synthesis of tert-butyl (2S,6R)-2-(hydroxymethyl)-6-methylmorpholine-4-carboxylate

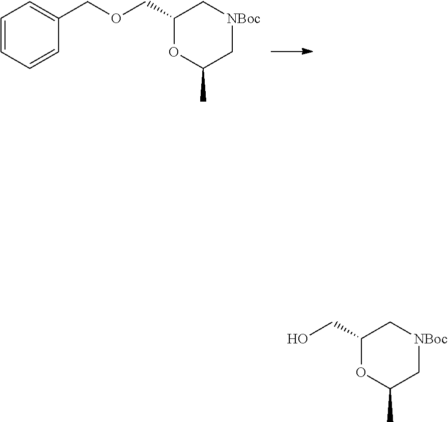 [SMILES: C[C@@H]1CN(C(=O)OC(C)(C)C)C[C@@H](COCc2ccccc2)O1 ; C[C@@H]1CN(C(=O)OC(C)(C)C)C[C@@H](CO)O1]

To a solution of (tert-butyl (2S,6R)-2-[(benzyloxy)methyl]-6-methylmorpholine-4-carboxylate (5.0 g, 15.6 mmol) in methanol (32 mL) was added palladium(II) hydroxide (20% loading wet support, 1.0 g, 0.71 mmol) and the reaction was stirred at room temperature for 1.5 hours. The reaction was filtered through Celite® and the filtrate was concentrated. The crude material was purified by column chromatography on silica gel eluting with 15-25% ethyl acetate in hexane gradient to give tert-butyl (2S,6R)-2-(hydroxymethyl)-6-methylmorpholine-4-carboxylate (3.3 g, 14.0 mmol, 92% yield) as a yellow oil. 1H NMR (400 MHz, DMSO-d6) δ 4.68 (dd, 1H), 3.90-3.76 (m, 1H), 3.70-3.57 (m, 1H), 3.50-3.34 (m, 3H), 3.30-2.87 (m, 2H), 1.40 (s, 9H), 1.07 (d, 3H). LCMS (Method 4-Column 2): Retention Time=1.26 minutes, [(M−100)H]+=132.

### Synthesis of 3-(benzylamino)-2,2-dimethylpropan-1-ol

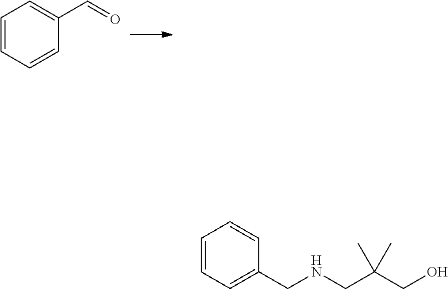

A solution of 3-amino-2,2-dimethylpropan-1-ol (10.0 g, 96.9 mmol) and benzaldehyde (10.8 g, 101.7 mmol) in benzene (170 mL) was refluxed for 4 hours using a Dean-Stark apparatus to remove water. The reaction was evaporated to yield an oily intermediate. To a solution of the imine intermediate in methanol (150 mL) cooled to 0° C. was added sodium borohydride (5.5 g, 145.1 mmol) in three portions. The reaction was stirred at room temperature for 16 hours. The reaction mixture was quenched with sodium hydroxide solution (6 N aqueous, 25 mL) and the solvents were evaporated. Water was added to the residue and the products were extracted with diethyl ether. The combined organic layers were dried over anhydrous sodium sulfate and concentrated. The crude material was purified by column chromatography on neutral alumina eluting with 15-20% ethyl acetate in hexane gradient to give 3-(benzylamino)-2,2-dimethylpropan-1-ol (17.0 g, 62.0 mmol, 91% yield) as a yellow oil. 1H NMR (400 MHz, Chloroform-d) δ 7.45-7.23 (m, 5H), 3.79 (s, 2H), 3.51 (s, 2H), 2.66 (s, 2H), 0.95 (s, 6H). LCMS (Method 4-Column 7): Retention Time=1.15 minutes, [MH]+=194.

### Synthesis of 3-{benzyl[(2S)-3-(benzyloxy)-2-hydroxypropyl]amino}-2,2-dimethpropan-1-ol

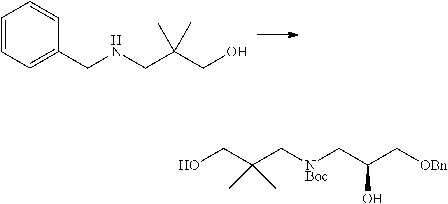 [SMILES: CC(C)(CO)CNCc1ccccc1 ; CC(C)(CO)CN(C[C@H](O)COCc1ccccc1)C(=O)OC(C)(C)C]

To a solution of 3-(benzylamino)-2,2-dimethylpropan-1-ol (17.0 g, 87.9 mmol) in 2-propanol (200 mL) was added (2S)-2-[(benzyloxy)methyl]oxirane (15.8 g, 96.4 mmol) and the reaction was heated to 50° C. for 16 hours. The volatiles were removed under reduced pressure and the crude material was purified by column chromatography on silica gel eluting with 15-20% ethyl acetate in hexane gradient to give 3-{benzyl[(2S)-3-(benzyloxy)-2-hydroxypropyl]amino}-2,2-dimethylpropan-1-ol (25.0 g, 69.4 mmol, 79% yield) as a viscous oil. 1H NMR (400 MHz, Chloroform-d) δ 7.46-7.20 (m, 10H), 4.51 (s, 2H), 4.06-3.96 (m, 1H), 3.80 (d, 1H), 3.58 (d, 1H), 3.47-3.32 (m, 4H), 2.71-2.45 (m, 4H), 0.99 (s, 3H), 0.90 (s, 3H). LCMS (Method 4-Column 7): Retention Time=1.49 minutes, [MH]+=358.

### Synthesis of 3-{benzyl[(2S)-3-(benzyloxy)-2-hydroxypropyl]amino}-2,2-dimethylpropyl methanesulfonate

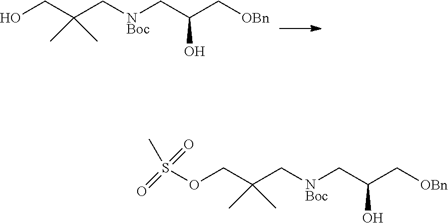 [SMILES: CC(C)(CO)CN(C[C@H](O)COCc1ccccc1)C(=O)OC(C)(C)C ; CC(C)(COS(C)(=O)=O)CN(C[C@H](O)COCc1ccccc1)C(=O)OC(C)(C)C]

Note: The reaction was performed as 6×1 g parallel experiments. To a solution of 3-{benzyl[(2S)-3-(benzyloxy)-2-hydroxypropyl]amino}-2,2-dimethylpropan-1-ol (1.0 g, 2.8 mmol) in dichloromethane (6.7 mL) cooled to 0° C. was slowly added N,N-diisopropylamine (0.5 mL, 2.8 mmol) and methanesulfonyl chloride (217 μL, 2.8 mmol). The reaction was stirred at 0° C. for 45 minutes and then was poured onto an aqueous saturated sodium bicarbonate solution and the products were extracted with dichloromethane. The combined organic layers from the 6 separate reactions were dried over anhydrous sodium sulfate and concentrated to give 3-{benzyl[(2S)-3-(benzyloxy)-2-hydroxypropyl]amino}-2,2-dimethylpropyl methanesulfonate (7.5 g, 17.2 mmol, 102% yield) as a viscous oil. No purification was carried out, the material was used crude in the next step. LCMS (Method 4-Column 7): Retention Time=1.89 minutes, [MH]+=436.

### Synthesis of (2S)-4-benzyl-2-[(benzyloxy)methyl]-6,6-dimethyl-1,4-oxazepane

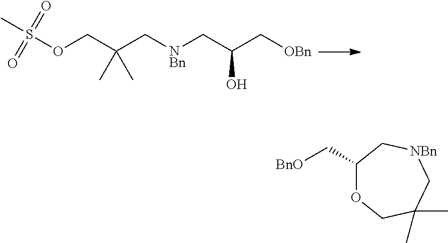 [SMILES: CC(C)(COS(C)(=O)=O)CN(Cc1ccccc1)C[C@H](O)COCc1ccccc1 ; CC1(C)CO[C@H](COCc2ccccc2)CN(Cc2ccccc2)C1]

Note: The reaction was performed as 7×1 g parallel experiments. To a solution of 3-{benzyl[(2S)-3-(benzyloxy)-2-hydroxypropyl]amino}-2,2-dimethylpropyl methanesulfonate (1.0 g, 2.3 mmol) in tetrahydrofuran (5 mL) cooled to 0° C. was added sodium hydride (57-63% w/w oil dispersion, 129 mg, 3.2 mmol) portion-wise. The reaction was stirred at room temperature for 16 hours. The reaction mixture was poured onto water and the products were extracted with ethyl acetate. The combined organic layers were dried over anhydrous sodium sulfate and concentrated. The combined crude material was purified by column chromatography on silica gel eluting with 1% ethyl acetate in hexane to give (2S)-4-benzyl-2-[(benzyloxy)methyl]-6,6-dimethyl-1,4-oxazepane (0.9 g, 2.7 mmol, 17% yield) as a viscous oil. 1H NMR (400 MHz, DMSO-d6) δ 7.48-7.07 (m, 10H), 4.43-4.29 (m, 2H), 3.73-3.47 (m, 3H), 3.41-3.22 (m, 4H), 2.84-2.70 (m, 1H), 2.43-2.20 (m, 3H), 0.80 (s, 3H), 0.69 (s, 3H). LCMS (Method 9): Retention Time=3.18 minutes, [MH]+=340.

### Synthesis of tert-butyl (2S)-2-(hydroxymethyl)-66-dimethyl-1,4-oxazepane-4-carboxylate

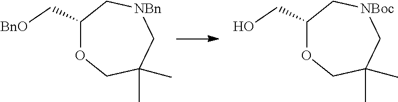 [SMILES: CC1(C)CO[C@H](COCc2ccccc2)CN(Cc2ccccc2)C1 ; CC1(C)CO[C@H](CO)CN(C(=O)OC(C)(C)C)C1]

To a solution of (2S)-4-benzyl-2-[(benzyloxy)methyl]-6,6-dimethyl-1,4-oxazepan (1.5 g, 4.4 mmol) in ethanol (30 mL) was added di-tert-butyl dicarbonate (1.2 g, 5.3 mmol) and palladium on carbon (10% w/w, 2.0 g, 1.9 mmol). The reaction was stirred in a hydrogenator at 200 psi for 24 hours. The reaction was filtered through Celite® eluting with methanol and the filtrate was concentrated under reduced pressure. The crude material was purified by column chromatography on silica gel eluting with 25% ethyl acetate in hexane to give (tert-butyl (2S)-2-(hydroxymethyl)-6,6-dimethyl-1,4-oxazepane-4-carboxylate (1.0 g, 3.9 mmol, 87% yield) as a viscous oil. 1H NMR (400 MHz, DMSO-d6) δ 4.79-4.60 (m, 1H), 3.78-3.36 (m, 4H), 3.31-2.96 (m, 4H), 1.39 (s, 9H), 0.97-0.63 (m, 6H). LCMS (Method 4-Column 7): Retention Time=1.56 minutes, [(M-56)H]+=204.

### Synthesis of 2-ethenyl-3-phenyloxirane

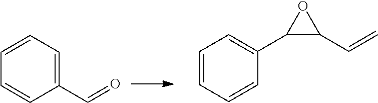

The reaction was performed in five parallel batches combined during workup.

To a solution of benzaldehyde (960 μL, 9.4 mmol) in tert-butanol (7 mL) were added allylbromide (2.4 mL, 28.3 mmol), potassium carbonate (6.5 g, 47.1 mmol) and tetrahydrothiophene (84 μL, 0.94 mmol). The reaction mixture was heated to reflux for 16 hours. The reaction mixture was cooled to room temperature, filtered and the products were extracted with hexane. The combined organic layers were dried over anhydrous sodium sulfate and concentrated under reduced pressure to give 2-ethenyl-3-phenyloxirane as a mixture of the cis and trans isomers (6.0 g, 41.0 mmol, 87% yield) as a pale-yellow liquid. The crude material was used for the next step without purification. 1H NMR (400 MHz, Chloroform-d) (1:1 mixture of cis and trans isomers) δ 7.38-7.27 (m, 5H), 5.78-5.66 (m, 1H), 5.59-5.49 (m, 1H), 5.37-5.25 (m, 1H), 4.25 (d, 0.5H), 3.78 (d, 0.5H), 3.67 (dd, 0.5H), 3.37 (dd, 0.5H).

### Synthesis of rel-(1R,2R)-2-aminocyclopentan-1-ol

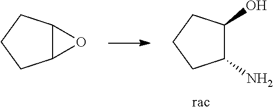 [SMILES: C1CC2OC2C1 ; N[C@@H]1CCC[C@H]1O]

To a solution of 6-oxabicyclo[3.1.0]hexane (10.0 g, 118.9 mmol) in ethanol (33 mL) was added ammonium hydroxide (25-30% in water, 200 mL, 118.9 mmol). The reaction mixture was stirred at room temperature for 24 hours. The volatiles were removed under reduced pressure to give rel-(1R, 2R)-2-aminocyclopentan-1-ol (8.8 g, 73.9 mmol, 62% yield) as a pale yellow oil. No purification was carried out and the material was used crude in the next step. 1H NMR (400 MHz, DMSO-d6) δ 3.84-3.68 (m, 1H), 3.05-2.83 (m, 1H), 1.98-1.77 (m, 2H), 1.71-1.55 (m, 2H), 1.54-1.38 (m, 1H), 1.32-1.17 (m, 1H). LCMS (Method 4-Column 3): Retention Time=0.70 minutes, [MH]+=102.

### Synthesis of N-[rel-(1R,2R)-2-hydroxycyclopentyl]acetamide

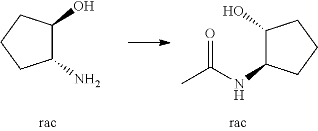

To a solution of rel-(1R,2R)-2-aminocyclopentan-1-ol (5.3 g, 52.4 mmol) in tetrahydrofuran (100 mL) cooled to 0° C., was added triethylamine (7.3 mL, 52.4 mmol), followed by dropwise addition of a solution of acetyl chloride (4.1 g, 52.4 mmol) in tetrahydrofuran (50 mL). The reaction was stirred at room temperature for 16 hours. The reaction mixture was filtered and the volatiles were removed under reduced pressure to give N-[rel-(1R,2R)-2-hydroxycyclopentyl]acetamide (8.3 g, 48.7 mmol, 93% yield) as a pale yellow oil. No purification was carried out and the material was used crude in the next step. 1H NMR (400 MHz, Deuterium Oxide) δ 4.01-3.90 (m, 1H), 3.90-3.79 (m, 1H), 2.07-1.94 (m, 1H), 1.91 (s, 3H), 1.88-1.79 (m, 1H), 1.74-1.59 (m, 2H), 1.59-1.45 (m, 1H), 1.45-1.31 (m, 1H). LCMS (Method 4-Column 2): Retention Time=0.69 minutes, [MH]+=144.

### Synthesis of rel-(3aS,6aR)-2-methyl-3aH,4H,5H,6H,6aH-cyclopenta[d][1,3]oxazole

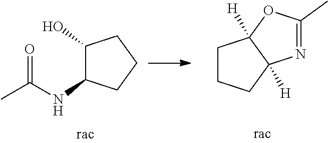

A solution of N-[rel-(1R,2R)-2-hydroxycyclopentyl]acetamide (11.3 g, 78.9 mmol) in chloroform (50 mL) was added dropwise to a flask containing thionyl chloride (23.5 mL, 323.6 mmol), maintaining the temperature between −10° C. and −5° C. under a nitrogen atmosphere. The reaction was stirred at room temperature for 16 hours. The reaction mixture was concentrated under reduced pressure to give rel-(3aS,6aR)-2-methyl-3aH,4H,5H,6H,6aH-cyclopenta[d][1,3]oxazole (15.5 g, 123.8 mmol, 157% yield). The crude material was used for next step. 1H NMR (400 MHz, DMSO-d6) δ 5.66 (dd, 1H), 4.83-4.68 (m, 1H), 2.38 (s, 3H), 2.11-1.96 (m, 1H), 1.92-1.64 (m, 4H), 1.66-1.48 (m, 1H). MS: [MH]+=126.

### Synthesis of rel-(1R,2S)-2-aminocyclopentan-1-ol hydrochloride

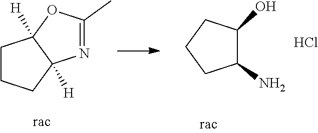

A solution of rel-(3aS,6aR)-2-methyl-3aH,4H,5H,6H,6aH-cyclopenta[d][1,3]oxazole (15.0 g, 119.8 mmol) in 10% aqueous hydrochloric acid (10.0 mL, 119.8 mmol) was heated to 100° C. for 1 hour. The reaction mixture was cooled to room temperature and concentrated under reduced pressure. The crude residue was triturated with methanol to give rel-(1R,2S)-2-aminocyclopentan-1-ol hydrochloride (9.6 g, 69.5 mmol, 58% yield) as a pale yellow solid used for the next step without further purification. 1H NMR (400 MHz, Deuterium Oxide) S 4.27-4.16 (m, 1H), 3.48-3.41 (m, 1H), 2.11-1.97 (m, 1H), 1.97-1.84 (m, 1H), 1.84-1.72 (m, 1H), 1.75-1.47 (m, 3H). MS: [MH]+=101.

### Synthesis of N-[rel-(1S,2R)-2-hydroxycyclopentyl]-4-methylbenzene-1-sulfonamide

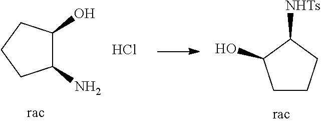

To a solution of rel-(1R,2S)-2-aminocyclopentan-1-ol hydrochloride (9.5 g, 69.0 mmol) in dichloromethane (15 mL) were added triethylamine (2.9 mL, 207.1 mmol), followed by p-toluenesulfonyl chloride (13.1 g, 69.0 mmol). The reaction was stirred at room temperature for 16 hours. The reaction mixture was poured into water and the products were extracted with dichloromethane. The organic layers were combined, dried over anhydrous sodium sulfate and concentrated. The crude material was purified by column chromatography on silica gel eluting with 2% methanol in dichloromethane to give N-[rel-(1S,2R)-2-hydroxycyclopentyl]-4-methylbenzenesulfonamide (10.4 g, 39.1 mmol, 57% yield) as a pale yellow solid. 1H NMR (400 MHz, DMSO-d6) S 7.72 (d, 2H), 7.36 (d, 2H), 7.21 (d, 1H), 4.62 (d, 1H), 3.76-3.64 (m, 11H), 3.20 (ddd, 1H), 2.37 (s, 3H), 1.68-1.49 (m, 2H), 1.49-1.19 (m, 4H). LCMS (Method 4-Column 2): Retention Time=1.91 minutes, [MH]+=256.

### Synthesis of rel-(2R,4aS,7aR)-4-(4-methylbenzenesulfonyl)-2-[(1E)-2-phenylethenyl]-octahydrocyclopenta[b][1,4]oxazine and rel-(2R,4aR,7aS)-4-(4-methylbenzenesulfonyl)-2-[(1E)-2-phenylethenyl]-octahydrocyclopenta[b][1,4]oxazine

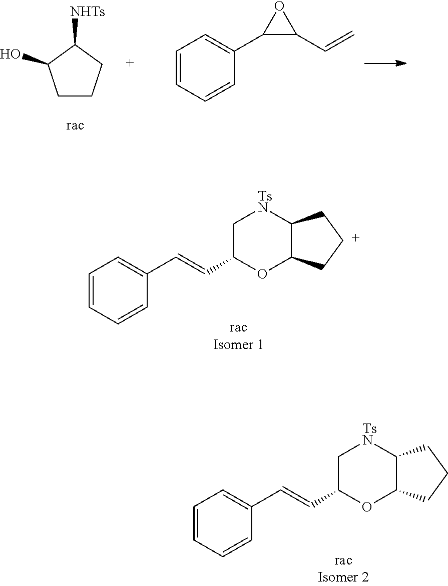

To a degassed solution of 2-ethenyl-3-phenyloxirane (7.4 g, 50.9 mmol) and N-[rel-(1R,2S)-2-hydroxycyclopentyl]-4-methylbenzenesulfonamide (10.0 g, 39.2 mmol) in dichloromethane (30 mL) was added palladium-tetrakis(triphenylphosphine) (450 mg, 0.39 mmol) and the reaction mixture was stirred at room temperature for 16 hours. Iron(III) chloride hexahydrate (1.1 g, 3.9 mmol) was added under inert atmosphere and the reaction mixture was stirred at room temperature for a further 16 hours. The reaction mixture was poured into water and the products extracted with dichloromethane. The combined organic layers were dried over anhydrous sodium sulfate and concentrated. The resulting crude material was purified by column chromatography on silica gel eluting with 6% ethyl acetate in n-hexane to give a mixture of the two diastereomeric products, which were triturated with a n-hexane and dichloromethane mixture to give Isomer 1: rel-(2R,4aS,7aR)-4-(4-methylbenzenesulfonyl)-2-[(1E)-2-phenylethenyl]-octahydrocyclopenta[b][1,4]oxazine (2.9 g, 6.4 mmols, 16% yield) as a yellow solid. The filtrate was concentrated under reduced pressure to give Isomer 2: (2S,4aR,7aS)-4-(4-methylbenzenesulfonyl)-2-[(1E)-2-phenylethenyl]-octahydrocyclopenta[b][1,4]oxazine (6.2 g, 13.6 mmol, 35% yield) as a yellow oil.

### Isomer 1: rel-(2R,4aS,7aR)-4-(4-methylbenzenesulfonyl)-2-[(1E)-2-phenylethenyl]-octahydrocyclopenta[b][1,4]oxazine

1H NMR (400 MHz, DMSO-d6) δ 7.71 (d, 2H), 7.40 (d, 2H), 7.37-7.31 (m, 4H), 7.31-7.23 (m, 1H), 6.61 (dd, TH), 6.36 (dd, 1H), 4.54-4.45 (m, 1H), 4.17-4.10 (m, 1H), 3.93-3.81 (m, 1H), 3.44 (dd, 1H), 3.22 (dd, 1H), 2.38 (s, 3H), 1.82-1.60 (m, 2H), 1.58-1.36 (m, 4H). LCMS (Method 4-Column 2): Retention Time=2.65 minutes, [MH]+=384.

### Isomer 2: rel-(2R,4aS,7aS)-4-(4-methylbenzenesulfonyl)-2-[(1E)-2-phenylethenyl]-octahydrocyclopenta[b][1,4]oxazine

1H NMR (400 MHz, DMSO-d6) δ 7.73 (d, 2H), 7.48-7.22 (m, 7H), 6.65 (d, 1H), 6.26 (dd, 1H), 4.08-3.88 (m, 2H), 3.88-3.74 (m, 1H), 3.59 (dd, 1H), 2.70 (dd, 1H), 2.40 (s, 3H), 1.86-1.60 (m, 2H), 1.63-1.44 (m, 2H), 1.44-1.26 (m, 2H). LCMS (Method 4-Column 2): Retention Time=2.73 minutes, [MH]+=384.

### Synthesis of [rel-(2S,4aR,7aS)-4-(4-methylbenzenesulfonyl)-octahydrocyclopenta[b],[1,4]oxazin-2-yl]methanol

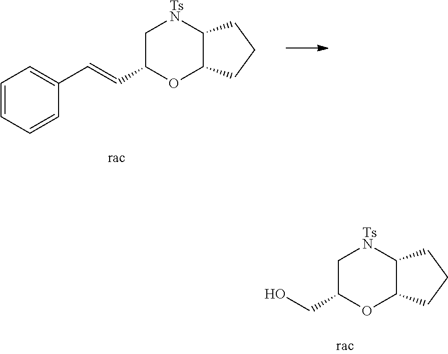 [SMILES: Cc1ccc(S(=O)(=O)N2C[C@@H](/C=C/c3ccccc3)O[C@H]3CCC[C@H]32)cc1 ; Cc1ccc(S(=O)(=O)N2C[C@@H](CO)O[C@H]3CCC[C@H]32)cc1]

To a solution of rel-(2R,4aR,7aS)-4-(4-methylbenzenesulfonyl)-2-[(1E)-2-phenylethenyl]-octahydrocyclopenta[b][1,4]oxazine (5.5 g, 14.3 mmol) in water (10 mL) and acetone (80 mL) was added N-methylmorpholine N-oxide (3.4 g, 28.7 mmol), followed by osmium tetroxide (109 mg, 0.43 mmol). The reaction was stirred at room temperature for 16 hours. The reaction mixture was quenched by addition of a saturated aqueous sodium thiosulfate solution (70 mL) and the products were extracted with ethyl acetate. The combined organic layers were dried over anhydrous sodium sulfate and concentrated to give 200 mg of crude intermediate. The diol intermediate was re-dissolved in water (40 mL) and ethanol (50 mL) and sodium periodate (18.4 g, 86.0 mmol) was added. The reaction mixture was stirred at room temperature for 16 hours. The reaction mixture was poured into water and the products were extracted with diethyl ether. The combined organic layers were dried over anhydrous sodium sulfate and concentrated. The resulting residue was dissolved in ethanol (50 mL) and sodium borohydride (2.2 g, 57.4 mmol) was added. The reaction mixture was stirred at room temperature for 16 hours. The volatiles were removed under reduced pressure and the residue was poured into water and extracted with ethyl acetate. The combined organic layers were dried over anhydrous sodium sulfate and concentrated. The resulting crude material was purified by column chromatography on silica gel eluting with 25% ethyl acetate in n-hexane to give [rel-(2S,4aR,7aS)-4-(4-methylbenzenesulfonyl)-octahydrocyclopenta[b][1,4]oxazin-2-yl]methanol (2.5 g, 7.7 mmol, 54% yield) as a colourless oil. 1H NMR (400 MHz, DMSO-d6) δ 7.68 (d, 2H), 7.42 (d, 2H), 4.85-4.74 (m, 1H), 3.98-3.83 (m, 1H), 3.77-3.66 (m, 1H), 3.54 (dd, 1H), 3.44-3.38 (m, 1H), 3.32-3.23 (m, 2H), 2.64-2.55 (m, 1H), 2.40 (s, 3H), 1.78-1.55 (m, 2H), 1.53-1.25 (m, 4H). LCMS (Method 8-Column 2): Retention Time=8.38 minutes, [MH]+=312.

### Synthesis of [rel-(2R,4aS,7aR)-4-(4-methylbenzenesulfonyl)-octahydrocyclopenta[b][1,4]oxazin-2-yl]methanol

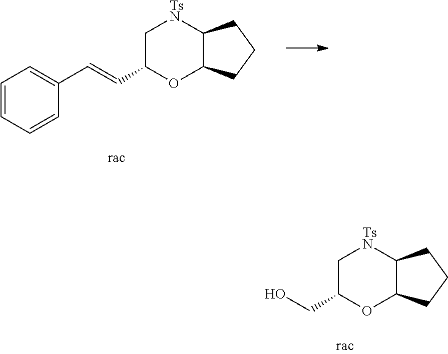 [SMILES: Cc1ccc(S(=O)(=O)N2C[C@@H](/C=C/c3ccccc3)O[C@@H]3CCC[C@@H]32)cc1 ; Cc1ccc(S(=O)(=O)N2C[C@@H](CO)O[C@@H]3CCC[C@@H]32)cc1]

To a solution of rel-(2S,4aS,7aR)-4-(4-methylbenzenesulfonyl)-2-[(1E)-2-phenylethenyl]-octahydrocyclopenta[b][1,4]oxazine (2.5 g, 6.5 mmol) in water (5 mL) and acetone (40 mL) was added N-methylmorpholine N-oxide (1.5 g, 13.0 mmol), followed by osmium tetroxide (50 mg, 0.20 mmol). The reaction was stirred at room temperature for 16 hours. The reaction mixture was quenched by addition of a saturated aqueous sodium thiosulfate solution (70 mL) and the products were extracted with ethyl acetate. The combined organic layers were dried over anhydrous sodium sulfate and concentrated to give 200 mg of crude intermediate. The diol intermediate was dissolved in water (20 mL) and ethanol (30 mL) and sodium periodate (8.3 g, 39.1 mmol) was added. The reaction mixture was stirred at room temperature for 16 hours. The reaction mixture was poured into water and the products were extracted with diethyl ether. The combined organic layers were dried over anhydrous sodium sulfate and concentrated. The resulting residue was dissolved in ethanol (30 mL) and sodium borohydride (990 mg, 26.1 mmol) was added. The reaction was stirred at room temperature for 16 hours. The volatiles were removed under reduced pressure and the crude residue was poured into water and the products were extracted with ethyl acetate. The combined organic layers were dried over anhydrous sodium sulfate and concentrated. The crude material was purified by column chromatography on silica gel eluting with 25% ethyl acetate in n-hexane to give [rel-(2R,4aS,7aR)-4-(4-methylbenzenesulfonyl)-octahydrocyclopenta[b][1,4]oxazin-2-yl]methanol (1.6 g, 4.7 mmol, 73% yield) as a colourless oil. 1H NMR (400 MHz, DMSO-d6) δ 7.67 (d, 2H), 7.41 (d, 2H), 4.73 (dd, 1H), 3.98-3.93 (m, 1H), 3.78-3.64 (m, 2H), 3.58-3.46 (m, 2H), 3.32-3.26 (m, 1H), 3.04 (dd, 1H), 2.40 (s, 3H), 1.74-1.57 (m, 2H), 1.52-1.34 (m, 4H). LCMS (Method 8-Column 2): Retention Time=8.33 minutes, [MH]+=312.

### Synthesis of [rel-(2S,4aR,7aS)-octahydrocyclopenta[b][1,4]oxazin-2-yl]methanol

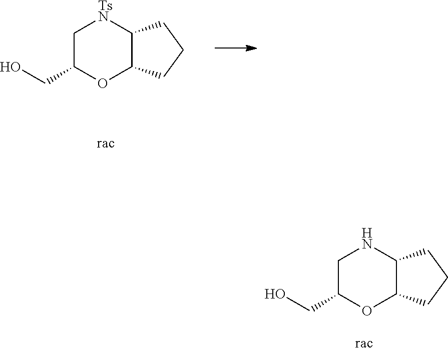 [SMILES: Cc1ccc(S(=O)(=O)N2C[C@@H](CO)O[C@H]3CCC[C@H]32)cc1 ; OC[C@@H]1CN[C@@H]2CCC[C@@H]2O1]

To a solution of [rel-(2S,4aR,7aS)-4-(4-methylbenzenesulfonyl)-octahydrocyclopenta[b][1,4]oxazin-2-yl]methanol (2.4 g, 7.7 mmol) in hydrobromic acid (30-33% in acetic acid, 84 mL) was added phenol (2.2 g, 23.1 mmol) and the reaction was stirred at room temperature for 16 hours. The reaction mixture was cooled to 0° C., basified to pH=8 by portion-wise addition of solid sodium hydroxide and concentrated. The products were extracted with a mixture of ethyl acetate and dichloromethane (1:1), followed by a mixture of methanol and dichloromethane (1:9). The combined organic layers were dried over anhydrous sodium sulfate and concentrated to give crude [rel-(2S,4aR,7aS)-octahydrocyclopenta[b][1,4]oxazin-2-yl]methanol (1.3 g, 8.3 mmol, 107% yield) as a pale yellow oil. The crude material was used in the next step without further purification. MS: [MH]+=158.

### Synthesis of [rel-(2S,4aS,7aR)-octahydrocyclopenta[b][1,4]oxazin-2-yl]methanol

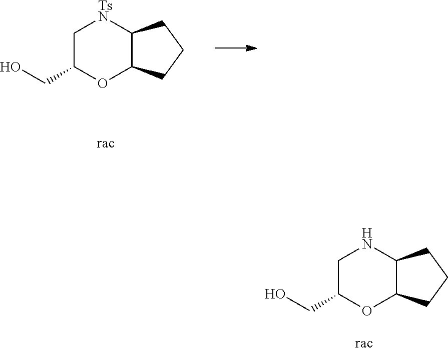 [SMILES: Cc1ccc(S(=O)(=O)N2C[C@@H](CO)O[C@@H]3CCC[C@@H]32)cc1 ; OC[C@@H]1CN[C@H]2CCC[C@H]2O1]

To a solution of [rel-(2S,4aS,7aR)-4-(4-methylbenzenesulfonyl)-octahydrocyclopenta[b][1,4]oxazin-2-yl]methanol (1.4 g, 4.5 mmol) in hydrobromic acid (30-33% in acetic acid, 50 mL) was added phenol (1.3 g, 13.5 mmol) and the reaction was stirred at room temperature for 16 hours. The reaction mixture was cooled to 0° C., basified to pH=8 by portion-wise addition of solid sodium hydroxide and concentrated under reduced pressure. The products were extracted with a mixture of ethyl acetate and dichloromethane (1:1), followed by a mixture of methanol and dichloromethane (1:9). The combined organic layers were dried over anhydrous sodium sulfate and concentrated to give crude [rel-(2S,4aS,7aR)-octahydrocyclopenta[b][1,4]oxazin-2-yl]methanol (5.0 g, 31.8 mmol, 660% yield) as a yellow oil. The crude material was used in the next step without further purification. MS: [MH]+=158.

### Synthesis of tert-butyl rel-(2S,4aR,7aS)-2-(hydroxymethyl)-octahydrocyclopenta[b][1,4]oxazine-4-carboxylate

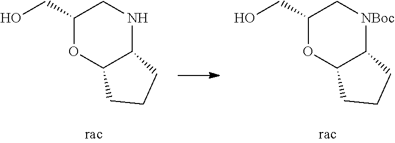 [SMILES: OC[C@@H]1CN[C@@H]2CCC[C@@H]2O1 ; CC(C)(C)OC(=O)N1C[C@@H](CO)O[C@H]2CCC[C@H]21]

To a solution of [rel-(2S,4aR,7aS)-octahydrocyclopenta[b][1,4]oxazin-2-yl]methanol (1.2 g, 7.6 mmol) in dichloromethane (50 mL) were added triethylamine (3.2 mL, 22.9 mmol) and 4-dimethylaminopyridine (93 mg, 0.76 mmol). The reaction mixture was stirred at room temperature for 5 minutes before addition of di-tert-butyl dicarbonate anhydride (1.7 g, 7.6 mmol) and the reaction mixture was stirred at room temperature for a further 4 hours. The reaction mixture was poured into water and the products were extracted with dichloromethane. The combined organic layers were dried over anhydrous sodium sulfate and concentrated under reduced pressure. The crude material was purified by column chromatography on silica gel eluting with 20% ethyl acetate in n-hexane to give tert-butyl rel-(2S,4aR,7aS)-2-(hydroxymethyl)-octahydrocyclopenta[b][1,4]oxazine-4-carboxylate (90 mg, 0.35 mmol, 5% yield) as a pale yellow oil. 1H NMR (400 MHz, Chloroform-d) δ 4.22-4.03 (m, 1H), 4.05-3.92 (m, 1H), 3.91-3.81 (m, 1H), 3.81-3.42 (m, 4H), 2.91-2.66 (m, 1H), 2.04-1.52 (m, 6H), 1.46 (s, 9H). LCMS (Method 4-Column 2): Retention Time=1.82 minutes, [(M−100)H]+=158. HPLC: Retention Time=6.61 minutes.

### Synthesis of tert-butyl rel-(2S,4aS,7aR)-2-(hydroxymethyl)-octahydrocyclopenta[b][1,4]oxazine-4-carboxylate

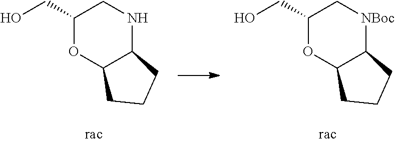 [SMILES: OC[C@@H]1CN[C@H]2CCC[C@H]2O1 ; CC(C)(C)OC(=O)N1C[C@@H](CO)O[C@@H]2CCC[C@@H]21]

To a solution of [rel-(2S,4aS,7aR)-octahydrocyclopenta[b][1,4]oxazin-2-yl]methanol (0.8 g, 5.1 mmol) in dichloromethane (30 mL) was added triethylamine (2.1 mL, 15.3 mmol) and 4-dimethylaminopyridine (62 mg, 0.51 mmol) and the reaction mixture was stirred at room temperature for 5 minutes before addition of di-tert-butyl dicarbonate anhydride (1.1 g, 5.1 mmol) and the reaction mixture was stirred at room temperature for a further 4 hours. The reaction mixture was poured into water and the products were extracted with dichloromethane. The combined organic layers were dried over anhydrous sodium sulfate and concentrated under reduced pressure. The crude material was purified by column chromatography eluting with 20% ethyl acetate in n-hexane to give tert-butyl rel-(2S,4aS,7aR)-2-(hydroxymethyl)-octahydrocyclopenta[b][1,4]oxazine-4-carboxylate (570 mg, 2.1 mmol, 41% yield) as a pale yellow oil. 1H NMR (400 MHz, Chloroform-d) δ 4.21-3.99 (m, 2H), 3.94-3.80 (m, 2H), 3.71-3.55 (m, 2H), 3.29 (d, J=13.9 Hz, 1H), 1.99-1.75 (m, 5H), 1.67-1.54 (m, 2H), 1.46 (s, 9H). LCMS (Method 4-Column 2): Retention Time=1.79 minutes, [(M-100)H]+=158. HPLC: Retention Time=6.40 minutes.

### Synthesis of oxiran-2-ylmethoxy-tri(propan-2-yl)silane

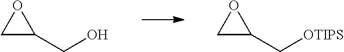

To a solution of oxiran-2-ylmethanol (20.0 g, 270.0 mmol) in N,N-dimethylformamide (150 mL) were added triisopropyl silyl chloride (58 mL, 270.0 mmol), triethyl amine (45 mL, 323.8 mmol) and N,N-dimethylamino pyridine (1.7 g, 13.5 mmol) and the reaction was stirred at room temperature for 4 hours. The reaction mixture was partitioned between water and diethyl ether. The products were extracted with diethyl ether and the combined organic layers were dried over anhydrous sodium sulfate and concentrated. The crude material was purified by column chromatography on silica gel eluting with 1% ethyl acetate in hexane to give oxiran-2-ylmethoxy-tri(propan-2-yl)silane (14.5 g, 62.9 mmol, 23% yield) as a colourless liquid. 1H NMR (400 MHz, Chloroform-d) δ 3.92 (dd, 1H), 3.75 (dd, 1H), 3.12 (ddd, 1H), 2.78 (dd, 1H), 2.67 (dd, 1H), 1.15-0.99 (m, 21H).

### Synthesis of benzyl(2,2-dimethoxyethyl)amine

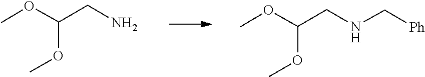

To a solution of 2,2-dimethoxyethanamine (15.0 g, 142.7 mmol) in methanol (150 mL) was added benzaldehyde (14.6 mL, 142.7 mmol) and the reaction was stirred at room temperature for 16 hours. The reaction mixture was cooled to 0° C. and sodium borohydride (8.1 g, 214.0 mmol) was added and the reaction was stirred at room temperature for a further 16 hours. 2 M aqueous hydrochloric acid solution was added to adjust to pH=9. The volatiles were removed under reduced pressure and the reaction mixture was diluted with water and the pH re-adjusted to pH=9. The products were extracted with dichloromethane. The combined organic layers were dried over anhydrous sodium sulfate and concentrated to give benzyl(2,2-dimethoxyethyl)amine (27.0 g, 138.3 mmol, 97% yield) as a colourless liquid. The material was used crude in the next step. 1H NMR (400 MHz, Chloroform-d) δ 7.35-7.29 (m, 4H), 7.27-7.22 (m, 1H), 4.49 (t, 1H), 3.80 (s, 2H), 3.36 (s, 6H), 2.75 (d, 2H). LCMS (Method 4-Column 7): Retention Time=1.21 minutes, [MH]+=196.

### Synthesis of 5-benzyl-3-methoxy-11-methyl-10,10-bis(propan-2-yl)-2,9-dioxa-5-aza-10-siladodecan-7-ol

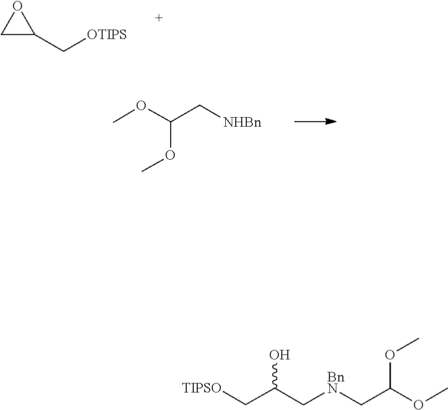

To a solution of oxiran-2-ylmethoxy-tri(propan-2-yl)silane (14.5 g, 62.9 mmol) in ethanol (150 mL) was added benzyl(2,2-dimethoxyethyl)amine (12.3 g, 62.9 mmol) The reaction was heated to 80° C. for 16 hours. The cooled reaction mixture was concentrated under reduced pressure to give 5-benzyl-3-methoxy-11-methyl-10,10-bis(propan-2-yl)-2,9-dioxa-5-aza-10-siladodecan-7-ol (26.0 g, 61.1 mmol, 97% yield) as a colourless liquid. The material was used crude in the next step. 1H NMR (400 MHz, Chloroform-d) δ 7.35-7.20 (m, 5H), 4.36 (t, 1H), 3.84-3.77 (m, 1H), 3.76-3.67 (m, 3H), 3.66-3.60 (m, 1H), 3.56-3.45 (m, 1H), 3.30 (s, 3H), 3.25 (s, 3H), 2.80-2.68 (m, 2H), 2.68-2.55 (m, 2H), 1.12-0.99 (m, 21H). LCMS (Method 4-Column 7): Retention Time=2.39 minutes, [MH]+=426.

### Synthesis of 4-benzyl-2-methoxy-6-({[tris(propan-2-yl)silyl]oxy}methyl)morpholine

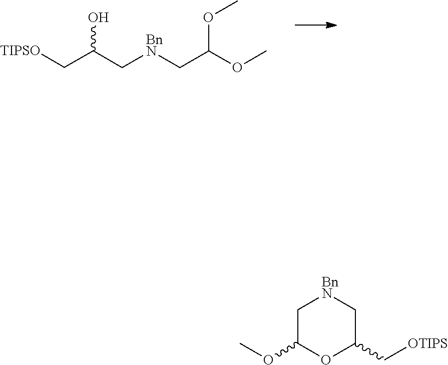

A mixture of 5-benzyl-3-methoxy-11-methyl-10,10-bis(propan-2-yl)-2,9-dioxa-5-aza-10-siladodecan-7-ol (26.0 g, 61.1 mmol) and para-toluenesulfonic acid (4.2 g, 24.4 mmol) was heated to 115° C. for 16 hours. The cooled reaction mixture was diluted with saturated aqueous sodium bicarbonate solution and the products were extracted with ethyl acetate. The organic layers were then dried over anhydrous sodium sulfate and concentrated. The crude product was purified by column chromatography on silica gel eluting with 10% ethyl acetate in n-hexane to give 4-benzyl-2-methoxy-6-({[tris(propan-2-yl)silyl]oxy}methyl)morpholine as 5:7 mixture of diastereoisomers (23.0 g, 58.4 mmol, 96% yield) as a pale yellow oil. 1H NMR (400 MHz, Chloroform-d) (5:7 mixture of diastereoisomers, but reporter as 1:1) S 7.35-7.27 (m, 5H), 4.50 (dd, 0.5H), 4.11-4.01 (m, 0.5H), 3.88-3.79 (m, 1H), 3.79-3.70 (m, 1H), 3.68-3.49 (m, 3H), 3.47 (s, 1.5H), 3.39 (s, 1.5H), 2.99-2.80 (m, 2H), 1.98-1.80 (m, 2H), 1.13-0.97 (m, 21H). LCMS (Method 4-Column 7): Retention Time=2.39 & 2.68 minutes, [MH]+=394.

### Synthesis of benzyl 2-({[tris(propan-2-yl)silyl]oxy}methyl)-3,4-dihydro-2H-oxazine-4-carboxylate

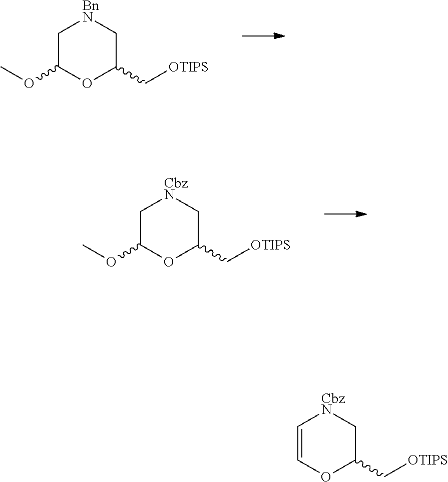

To a solution of 4-benzyl-2-methoxy-6-({[tris(propan-2-yl)silyl]oxy}methyl)morpholine (10.0 g, 25.4 mmol) in dichloromethane (100 mL) was added benzyl chloroformate (5.8 mL, 40.6 mmol) and the reaction was stirred at room temperature for 16 hours. The solvents were removed under reduced pressure and the residue was dissolved in toluene (800 mL) and p-toluenesulfonic acid (1.7 g, 10.2 mmol) was added. The reaction mixture was heated to reflux with a Dean-Stark apparatus for 2 hours. The cooled reaction mixture was quenched by addition of saturated aqueous sodium bicarbonate solution and the products were extracted with diethyl ether. The organic layers were combined, dried over anhydrous sodium sulfate and concentrated. The crude material was purified by column chromatography on silica gel eluting with 5% ethyl acetate in hexane to give benzyl 2-methoxy-6-({[tris(propan-2-yl)silyl]oxy)}methyl)morpholine-4-carboxylate (4.8 g, 11.8 mmol, 49% yield) as a pale yellow oil. 1H NMR (400 MHz, Chloroform-d) mixture of rotamers S 7.42-7.30 (m, 5H), 6.31 (d, 05H), 6.20 (dd, 0.5H), 6.01 (d, 0.5H), 5.90 (d, 0.5H), 5.27-5.05 (m, 2H), 4.21-4.09 (m, 0.5H), 4.09-4.01 (m, 0.5H), 4.01-3.93 (m, 1H), 3.93-3.83 (m, 1H), 3.83-3.66 (m, 1H), 3.43 (dd, 0.5H), 3.31 (dd, 0.5H), 1.10-1.00 (m, 21H). LCMS (Method 4-Column 2): Retention Time=3.59 minutes, [MH]+=406.

### Synthesis of benzyl 3-({[tris(propan-2-yl)silyl]oxy}methyl)-2-oxa-5-azabicyclo[4.1.0]heptane-5-carboxylate

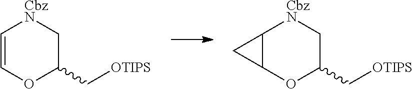

To a solution of benzyl 2-({[tris(propan-2-yl)silyl]oxy}methyl-3,4-dihydro-2H-oxazine-4-carboxylate (4.8 g, 11.8 mmol) in benzene (60 mL) cooled to 0° C. were added diiodomethane (47.6 g, 177.5 mmol) and diethyl zinc (1 M solution in hexanes) (177 mL, 177.5 mmol). The reaction mixture was warmed to room temperature and stirred for 3 hours. The reaction mixture was quenched by addition of aqueous saturated solution of sodium bicarbonate and the products were extracted with diethyl ether. The combined organic layers were dried over anhydrous sodium sulfate and concentrated. The crude product was purified by column chromatography on silica gel eluting with 5% ethyl acetate in hexane to give benzyl 3-({[tris(propan-2-yl)silyl]oxy}methyl)-2-oxa-5-azabicyclo[4.1.0]heptane-5-carboxylate (1.8 g, 4.3 mmol, 36% yield) as a colourless liquid. 1H NMR (400 MHz, Chloroform-d) δ 7.49-7.28 (m, 5H), 5.27-5.11 (m, 2H), 3.98-3.50 (m, 5H), 2.98-2.75 (m, 2H), 1.42-1.15 (m, 2H), 1.12-0.96 (m, 18H), 0.94-0.78 (m, 3H). LCMS (Method 4-Column 2): Retention Time=3.53 minutes, [MH]+=420.

### Synthesis of benzyl 3-(hydroxymethyl)-2-oxa-5-azabicyclo[4.1.0]heptane-5-carboxylate

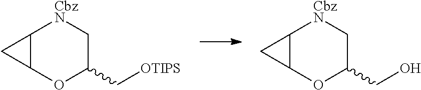

To a solution of benzyl 3-({[tris(propan-2-yl)silyl]oxy}methyl)-2-oxa-5-azabicyclo[4.1.0]heptane-5-carboxylate (2.8 g, 6.7 mmol) in tetrahydrofuran (140 mL) cooled to 0° C. was added dropwise tetra-n-butyl ammonium fluoride (TBAF, 1 M solution in tetrahydrofuran, 6.8 mL, 23.4 mmol). The reaction mixture was stirred for 3 hours and quenched by addition of saturated aqueous sodium bicarbonate solution and the products were extracted with ethyl acetate. The combined organic layers were dried over anhydrous sodium sulfate and concentrated. The crude material was purified by column chromatography on silica gel eluting with 35% ethyl acetate in hexane to give benzyl 3-(hydroxymethyl)-2-oxa-5-azabicyclo[4.1.0]heptane-5-carboxylate (1.7 g, 6.5 mmol, 97% yield) as a colourless oil. 1H NMR (400 MHz, Chloroform-d) δ 7.44-7.30 (m, 5H), 5.27-5.11 (m, 2H), 3.79-3.73 (m, 1H), 3.72-3.60 (m, 3H), 2.97-2.76 (m, 2H), 1.96-1.84 (m, 1H), 0.88-0.80 (m, 2H). LCMS (Method 4-Column 7): Retention Time=1.37 minutes, [MH]+=264.

### Synthesis of rel-(2S,3R)-3-aminobutan-2-ol

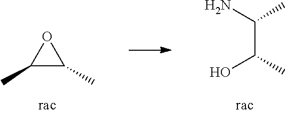

Rel-(2R,3R)-2,3-dimethyloxirane (2.2 g, 30.5 mmol) was dissolved in ammonium hydroxide (28% in water, 12 mL, 30.5 mmol) and the reaction mixture was stirred at room temperature for 72 hours. The solvents were removed under reduced pressure to give crude rel-(2S,3R)-3-aminobutan-2-ol (2.0 g, 22.4 mmol, 74% yield) as a light-yellow oil and was used in the next step without purification. 1H NMR (400 MHz, Chloroform-d) δ 3.65 (qd, 1H), 2.92 (qd, 1H), 1.09 (d, 3H), 1.00 (d, 3H). LCMS (Method 9): Retention Time=0.32 minutes, [MH]+=90.

### Synthesis of N-[rel-(2S,3R)-3-hydroxybutan-2-yl]-4-methylbenzene-1-sulfonamide

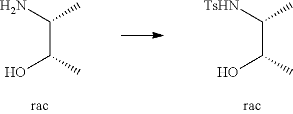

To the solution of rel-(2S,3R)-3-aminobutan-2-ol (6.0 g, 67.3 mmol) in dichloromethane (100 mL) was added triethyl amine (7.5 g, 74.0 mmol) at 0° C., followed by p-toluenesulfonyl chloride (12.8 g, 67.3 mmol) 15 minutes later. The reaction mixture was then allowed to warm to room temperature and stirring was continued for 48 hours. The reaction mixture was poured into water and the products extracted with dichloromethane. The combined organic layers were dried over anhydrous sodium sulfate and concentrated. The crude residue was purified by column chromatography eluting with 15% ethyl acetate in hexane to give N-[rel-(2S,3R)-3-hydroxybutan-2-yl]-4-methylbenzene-1-sulfonamide (9.0 g, 37.0 mmol, 55% yield) as a colourless solid. 1H NMR (400 MHz, Chloroform-d) δ 7.77 (d, 2H), 7.31 (d, 2H), 4.84 (d, 1H), 3.78 (qd, 1H), 3.29 (dqd, 1H), 2.43 (s, 3H), 1.10 (d, 3H), 0.95 (d, 3H). LCMS (Method 4-Column 1): Retention Time=1.51 minutes, [MH]+=244.

### Synthesis of rel-(2S,3R,6S)-2,3-dimethyl-4-(4-methylbenzenesulfonyl)-6-[(1E)-2-phenylethenyl]morpholine and rel-(2S,3R,6R)-2,3-dimethyl-4-(4-methylbenzenesulfonyl)-6-[(1E)-2-phenylethenyl]morpholine

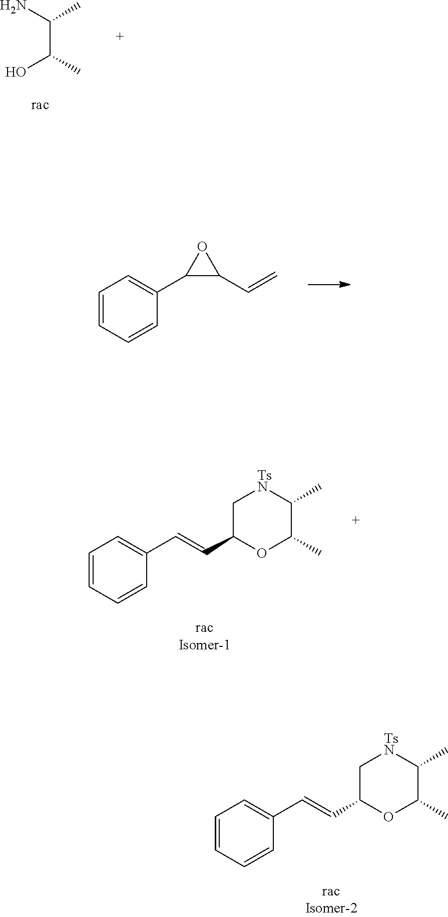

To a degassed solution of N-[rel-(2S,3R)-3-hydroxybutan-2-yl]-4-methylbenzene-1-sulfonamide (6.0 g, 24.7 mmol) and 2-ethenyl-3-phenyloxirane (4.7 g, 32.1 mmol) in dichloromethane (15 mL) was added tetrakis(triphenylphosphine)palladium (280 mg, 0.25 mmol) and the reaction was stirred at room temperature for 16 hours. After this time, iron (III) trichloride hexahydrate (670 mg, 2.5 mmol) was added and the reaction was stirred for further 16 hours. The reaction mixture was poured into water and the products were extracted with ethyl acetate. The combined organic layers were dried over anhydrous sodium sulfate and concentrated. The crude residue was purified by column chromatography on silica gel eluting with 6-8% ethyl acetate in hexane to isolate both diastereoisomers.

### Isomer 1: rel-(2S,3R,6S)-2,3-dimethyl-4-(4-methylbenzenesulfonyl)-6-[(1E)-2-phenylethenyl]morpholine: obtained as a yellow liquid (3.0 g, 8.1 mmol, 33% yield)

1H NMR (400 MHz, Chloroform-d) δ 7.70 (d, 2H), 7.43-7.28 (m, 7H), 6.66 (d, 1H), 6.10 (dd, TH), 4.14 (ddd, 1H), 3.91-3.83 (m, 1H), 3.84-3.76 (m, 1H), 3.65 (dd, 1H), 2.87 (dd, 1H), 2.43 (s, 3H), 1.13 (d, 3H), 0.95 (d, 3H). LCMS (Method 4-Column 1): Retention Time=2.51 minutes, [MH]+=372.

### Isomer 2: rel-(2S,3R,6R)-2,3-dimethyl-4-(4-methylbenzenesulfonyl)-6-[(1E)-2-phenylethenyl]morpholine: obtained as a colourless solid (2.0 g, 5.4 mmol, 22% yield)

1H NMR (400 MHz, Chloroform-d) δ 7.70 (d, 2H), 7.38-7.21 (m, 7H), 6.62 (dd, 1H), 6.27 (dd, 1H), 4.53-4.43 (m, 1H), 4.14-4.03 (m, 1H), 3.90-3.78 (m, 1H), 3.59 (d, 1H), 3.39 (dd, 1H), 2.41 (s, 3H), 1.05 (d, 3H), 1.02 (d, 3H). LCMS (Method 4-Column 1): Retention Time=2.39 minutes, [MH]+=372.

### Synthesis of rel-(2S,3R,6S)-2,3-dimethyl-4-(4-methylbenzenesulfonyl)-6-[(1E)-2-phenylethenyl]morpholine

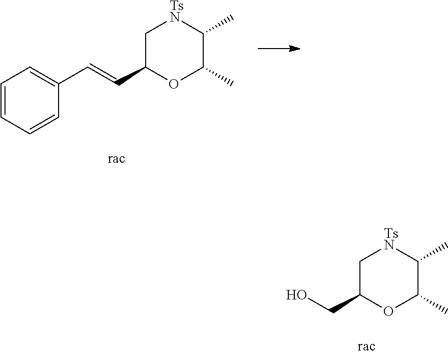 [SMILES: Cc1ccc(S(=O)(=O)N2C[C@H](/C=C/c3ccccc3)O[C@@H](C)[C@H]2C)cc1 ; Cc1ccc(S(=O)(=O)N2C[C@H](CO)O[C@@H](C)[C@H]2C)cc1]

To a solution of rel-(2S,3R,6S)-2,3-dimethyl-4-(4-methylbenzenesulfonyl)-6-[(1E)-2-phenylethenyl]morpholine (3.0 g, 8.1 mmol) in acetone (55 mL) and water (7 mL) were added N-methylmorpholine N-oxide (1.9 g, 16.1 mmol) and osmium tetroxide (2.5 g, 0.24 mmol) and the reaction was stirred at room temperature for 16 hours. The reaction was quenched by addition of a saturated aqueous solution of sodium thiosulfate and the products were extracted with ethyl acetate. The combined organic layers were dried over anhydrous sodium sulfate and concentrated. The crude diol intermediate was dissolved in water (27 mL) and ethanol (35 mL) and sodium periodate (10.4 g, 48.5 mmol) was added. The reaction was stirred at room temperature for 16 hours. The reaction mixture was poured into water and the products extracted with diethyl ether. The combined organic layers were dried over anhydrous sodium sulfate and concentrated. The crude material was dissolved in ethanol (50 mL) and sodium borohydride (1.2 g, 32.3 mmol) was added. The reaction mixture was stirred at room temperature for 16 hours. The volatiles were removed under reduced pressure and the crude material was poured into a saturated aqueous solution of ammonium chloride and the products were extracted with ethyl acetate. The combined organic layers were dried over anhydrous sodium sulfate and concentrated. The crude residue was purified by column chromatography on silica gel eluting with 19% ethyl acetate in hexane to give [(rel-2R,5R,6S)-5,6-dimethyl-4-(4-methylbenzenesulfonyl)morpholin-2-yl]methanol (1.8 g, 6.0 mmol, 74%) as a yellow oil. 1H NMR (400 MHz, DMSO-d6) δ 7.68 (d, 2H), 7.42 (d, 2H), 4.82-4.74 (m, 1H), 3.81-3.69 (m, 1H), 3.60-3.46 (m, 2H), 3.44-3.37 (m, 1H), 3.32-3.26 (m, 2H), 2.78-2.62 (m, 1H), 2.40 (s, 3H), 0.97 (d, 3H), 0.81 (d, 3H). LCMS (Method 4-Column 1): Retention Time=1.55 minutes, [MH]+=300.

### Synthesis of rel-(2S,3R,6R)-2,3-dimethyl-4-(4-methylbenzenesulfonyl)-6-[(1E)-2-phenylethenyl]morpholine

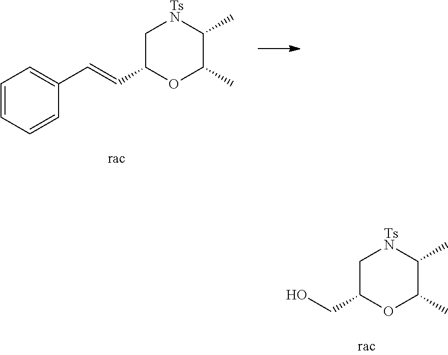 [SMILES: Cc1ccc(S(=O)(=O)N2C[C@@H](/C=C/c3ccccc3)O[C@@H](C)[C@H]2C)cc1 ; Cc1ccc(S(=O)(=O)N2C[C@@H](CO)O[C@@H](C)[C@H]2C)cc1]

To a solution of rel-(2S,3R,6R)-2,3-dimethyl-4-(4-methylbenzenesulfonyl)-6-[(1E)-2-phenylethenyl]morpholine (2.0 g, 5.4 mmol) in acetone (35 mL) and water (4.5 mL) was added N-methylmorpholine N-oxide (1.3 g, 10.8 mmol) and osmium tetroxide (1.6 g, 0.16 mmol) and the reaction was stirred at room temperature for 16 hours. The reaction was quenched by addition of a saturated aqueous solution of sodium thiosulfate and the products were extracted with ethyl acetate. The combined organic layers were dried over anhydrous sodium sulfate and concentrated. The crude diol intermediate was then dissolved in water (18 mL) and ethanol (23 mL) and sodium periodate (6.9 g, 32.3 mmol) was added. Reaction was stirred at room temperature for 16 hours. The reaction mixture was poured into water and the products were extracted with diethyl ether. The combined organic layers were dried over anhydrous sodium sulfate and concentrated. The crude material was then dissolved in ethanol (32 mL) and sodium borohydride (810 mg, 21.5 mmol) was added and the reaction was stirred at room temperature for 16 hours. The volatiles were removed under reduced pressure and the crude material was poured into a saturated aqueous solution of ammonium chloride and the products were extracted with ethyl acetate. The combined organic layers were dried over anhydrous sodium sulfate and concentrated. The crude residue was purified by column chromatography on silica gel on eluting with 21% ethyl acetate in hexane to give [rel-(2S,5R,6S)-5,6-dimethyl-4-(4-methylbenzenesulfonyl)morpholin-2-yl]methanol (1.2 g, 4.0 mmol, 74% yield) as a yellow oil. 1H NMR (400 MHz, DMSO-d6) δ 7.72-7.62 (m, 2H), 7.46-7.36 (m, 2H), 4.72-4.63 (m, 1H), 3.81-3.70 (m, 1H), 3.70-3.58 (m. 2H), 3.53-3.34 (m, 3H), 3.15-3.02 (m, 1H), 2.40 (s, 3H), 0.94-0.89 (m, 3H), 0.86-0.79 (m, 3H) LCMS (Method 4-Column 1): Retention Time=1.51 minutes, [MH]+=300.

### Synthesis of tert-butyl rel-(2S,3R,6R)-6-(hydroxymethyl)-2,3-dimethylmorpholine-4-carboxylate

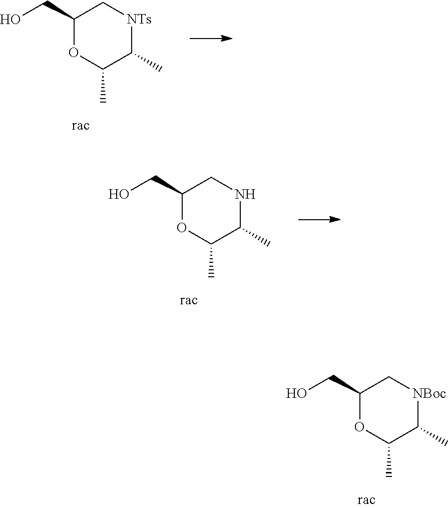

To a solution of [rel-(2R,5R,6S)-5,6-dimethyl-4-(4-methylbenzenesulfonyl)morpholin-2-yl]methanol (1.8 g, 6.0 mmol) in hydrobromic acid (30-33% solution in acetic acid, 40 mL, 6.0 mmol) was added (1.7 g, 18.0 mmol) was added and the reaction was stirred at room temperature for 16 hours. The reaction mixture was poured into water and the products were extracted with a 10% methanol in dichloromethane mixture. The combined organic layers were dried over anhydrous sodium sulfate and concentrated to give intermediate [rel-(2R,5R,6S)-5,6-dimethylmorpholin-2-yl]methanol (870 mg, 6.0 mmol) as a yellow oil, no purification was carried out. MS: [MH]+=146.5

To a solution of the crude intermediate in dichloromethane (40 mL) was added triethyl amine (1.8 g, 18.0 mmol) and 4-(dimethylamino)pyridine (70 mg, 0.6 mmol). The reaction was cooled to 0° C. and di-tert-butyl dicarbonate (1.3 g, 6.0 mmol) was added. The reaction mixture was warmed to room temperature and it was stirred for 16 hours. The reaction mixture was poured into water and the products were extracted with dichloromethane. The combined organic layers were dried over anhydrous sodium sulfate and concentrated. The crude residue was purified by column chromatography on silica gel eluting with 12% diethyl ether in petroleum ether to give tert-butyl rel-(2S,3R,6R)-6-(hydroxymethyl)-2,3-dimethylmorpholine-4-carboxylate (400 mg, 1.6 mmol, 27% yield) as a pale yellow oil. 1H NMR (400 MHz, Chloroform-d) δ 4.06-3.96 (m, 0.5H), 3.88-3.80 (m, 0.5H), 3.79-3.63 (m, 3H), 3.63-3.48 (m, 2H), 2.88 (dd, 0.5H), 2.76 (dd, 0.5H), 1.52-1.39 (m, 9H), 1.16-1.09 (m, 3H), 1.09-1.03 (m, 3H). LCMS (Method 4-Column 1): Retention Time=1.45 minutes, [MH]+=146. HPLC: Retention Time=5.99 minutes.

### Synthesis of tert-butyl rel-(2S,3R,6S)-6-(hydroxymethyl)-2,3-dimethylmorpholine-4-carboxylate

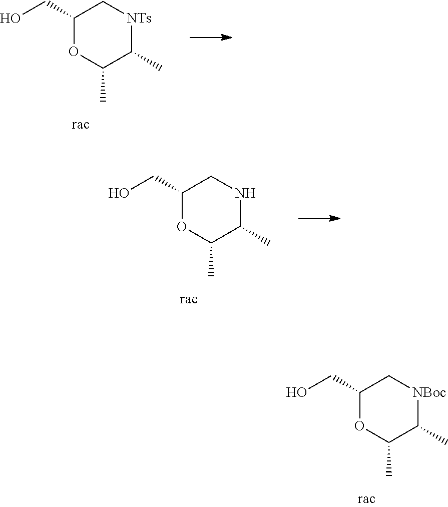

To a solution of [rel-(2S,5R,6S)-5,6-dimethyl-4-(4-methylbenzenesulfonyl)morpholin-2-yl]methanol (1.2 g, 4.0 mmol) in hydrobromic acid solution (30-33% in acetic acid, 30 mL, 4.0 mmol) was added phenol (1.1 g, 12.0 mmol) was added. The reaction was stirred at room temperature for 16 hours. On completion, the reaction mixture was poured into water and the products were extracted with a mixture of methanol in dichloromethane (10%). The combined organic layers were dried over anhydrous sodium sulfate and concentrated to give intermediate [rel-(2R,5R,6S)-5,6-dimethylmorpholin-2-yl]methanol (580.0 mg, 4.0 mmol) as a yellow liquid. No purification was carried out on the intermediate. MS: [MH]+=146.

To a solution of the crude intermediate in dichloromethane (40 mL) was added triethylamine (1.2 g, 12.0 mmol) and 4-(dimethylamino)pyridine (50.0 mg, 0.40 mmol). The reaction mixture was cooled to 0° C. before addition of di-tert-butyl dicarbonate (870 mg, 4.0 mmol). Following the addition, the reaction mixture was warmed to room temperature and was stirred for 16 hours. The reaction mixture was poured into water and the products were extracted with dichloromethane. The combined organic layers were dried over anhydrous sodium sulfate and concentrated. The crude residue was purified by column chromatography on silica gel eluting with 12% diethyl ether in petroleum ether to give tert-butyl tert-butyl rel-(2S,3R,6S)-6-(hydroxymethyl)-2,3-dimethylmorpholine-4-carboxylate (160 mg, 0.65 mmol, 16% yield) as a pale yellow oil. 1H NMR (400 MHz, Chloroform-d) δ 4.02-3.83 (m, 4H), 3.65 (d, 1H), 3.60-3.52 (m, 1H), 3.30 (dd, 1H), 1.46 (s, 9H), 1.15 (d, 3H), 1.09 (d, 3H). LCMS (Method 4-Column 1): Retention Time=1.43 minutes, [MH])=146. HPLC: Retention Time=5.73 minutes.

### Synthesis of ethyl 3-(benzylamino)-3-methylbutanoate

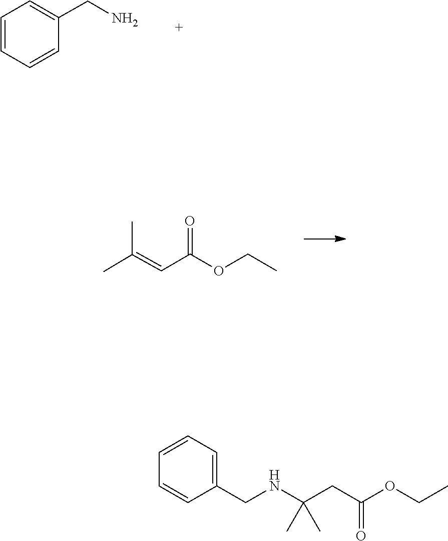

A solution of ethyl 3-methylbut-2-enoate (25.0 g, 195.1 mmol) and benzylamine (20.9 g, 195.1 mmol) in ethanol (200 mL) was heated to 90° C. for 48 hours. The reaction mixture was concentrated under reduced pressure and the crude material was purified by column chromatography on silica gel eluting with 20% ethyl acetate in hexane to give ethyl 3-(benzylamino)-3-methylbutanoate (12.0 g, 51.0 mmol, 26% yield) as a yellow oil. 1H NMR (400 MHz, Chloroform-d) δ 7.43-7.28 (m, 4H), 7.25-7.20 (m, 1H), 4.14 (q, 2H), 3.73 (s, 2H), 2.53 (s, 2H), 1.30-1.20 (m, 9H). LCMS (Method 4-Column 7): Retention Time=1.00 minutes, [MH]+=236.

### Synthesis of 3-(benzylamino)-3-methylbutan-1-ol

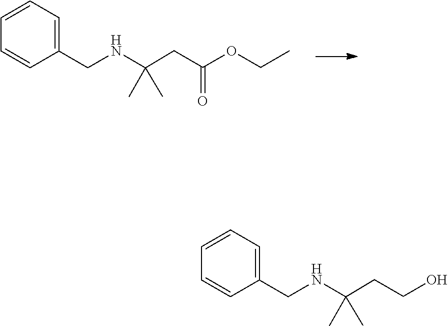 [SMILES: CCOC(=O)CC(C)(C)NCc1ccccc1 ; CC(C)(CCO)NCc1ccccc1]

To a solution of ethyl 3-(benzylamino)-3-methylbutanoate (12.0 g, 51.0 mmol) in tetrahydrofuran (250 mL), cooled to 0° C. under a nitrogen atmosphere, was added dropwise a solution of lithium aluminium hydride (2.5 M in tetrahydrofuran, 41 mL, 82.0 mmol). The reaction mixture warned to room temperature slowly and stirring was continued for 2 hours. The reaction mixture was cooled to 0° C. and quenched by addition of water. The mixture was filtered through Celite® washing with ethyl acetate. The products were extracted with ethyl acetate and the combined organic layers were dried over anhydrous sodium sulfate and concentrated to give 3-(benzylamino)-3-methylbutan-1-ol (9.5 g, 49.1 mmol, 96% yield) as an off-white solid. 1H NMR (400 MHz, Chloroform-d) δ 7.40-7.21 (m, 5H), 3.90-3.84 (m, 2H), 3.76 (s, 2H), 1.69-1.63 (m, 2H), 1.26 (s, 6H). LCMS (Method 4-Column 7): Retention Time=0.79 minutes, [MH]+=194.

### Synthesis of 3-{benzyl[(2S)-3-(benzyloxy)-2-hydroxypropyl]amino}-3-methylbutan-1-ol

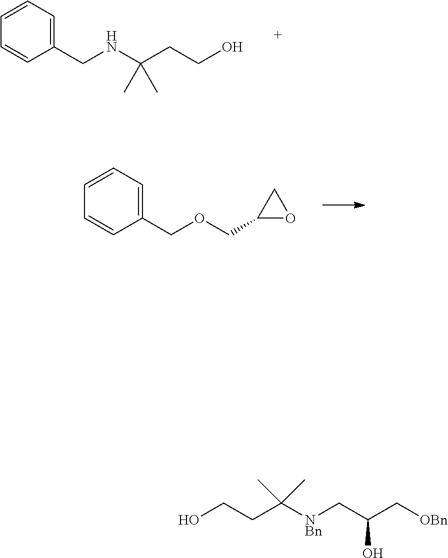 [SMILES: CC(C)(CCO)NCc1ccccc1 ; c1ccc(COC[C@@H]2CO2)cc1 ; CC(C)(CCO)N(Cc1ccccc1)C[C@H](O)COCc1ccccc1]

To a solution of 3-(benzylamino)-3-methylbutan-1-ol (9.5 g, 49.1 mmol) was added (2S)-2-(phenylmethoxymethyl)oxirane (9.7 g, 59.0 mmol) in 2-propanol (80 mL) and the reaction mixture was heated to 70° C. for 16 hours. After this time the reaction mixture was concentrated under reduced pressure and the resulting crude residue was purified by column chromatography on silica gel eluting with 2% methanol in dichloromethane to give 3-{benzyl[(2S)-3-(benzyloxy)-2-hydroxypropyl]amino}-3-methylbutan-1-ol (9.5 g, 26.6 mmol, 54% yield) as a yellow oil. 1H NMR (400 MHz, Chloroform-d) δ 7.39-7.18 (m, 10H), 4.48-4.34 (m, 2H), 4.06-3.90 (m, 2H), 3.88-3.72 (m, 1H), 3.61-3.47 (m, 1H), 3.42 (br s, 1H), 3.30-3.15 (m, 2H), 2.90 (dd, 1H), 2.60 (d, 1H), 2.10-1.95 (m, 1H), 1.61-1.51 (m, 1H), 1.22 (s, 3H), 1.16 (s, 3H). LCMS (Method 4-Column 1): Retention Time=1.48 minutes, [MH]+=358.

### Synthesis of (2S)-4-benzyl-2-[(benzyloxy)methyl]-5,5-dimethyl-1,4-oxazepane

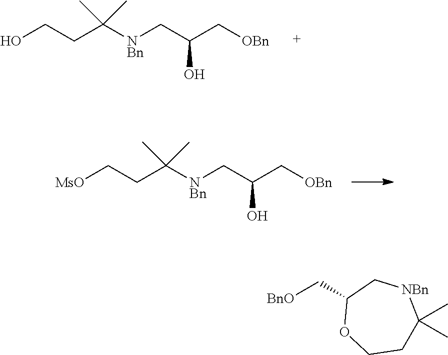 [SMILES: CC(C)(CCO)N(Cc1ccccc1)C[C@H](O)COCc1ccccc1 ; CC(C)(CCOS(C)(=O)=O)N(Cc1ccccc1)C[C@H](O)COCc1ccccc1 ; CC1(C)CCO[C@H](COCc2ccccc2)CN1Cc1ccccc1]

To a solution of 3-{benzyl[(2S)-3-(benzyloxy)-2-hydroxypropyl]amino}-3-methylbutan-1-ol (500 mg, 1.4 mmol) in dichloromethane (7 mL) cooled to 0° C., were added diisopropylethylamine (0.25 mL, 1.4 mmol) and methanesulfonyl chloride (110 μL, 1.4 mmol) and the reaction mixture was stirred for 15 minutes at 0° C. After this time, the reaction mixture was poured into water and the products extracted with dichloromethane. The combined organic layers were dried over anhydrous sodium sulfate and concentrated to give the crude mesylate intermediate as a red oil.

To a solution of the mesylate intermediate in tetrahydrofuran (5 mL) cooled to 0° C. was added portion-wise sodium hydride (60% dispersion in mineral oil, 90 mg, 2.2 mmol). The reaction mixture was warmed to room temperature and stirring was continued for 16 hours. On completion, the reaction mixture was poured into water and the products were extracted with ethyl acetate. The combined organic layers were dried over anhydrous sodium sulfate and concentrated. The two-step sequence described above was run in 16 parallel setups. The crude residues were combined and purified by column chromatography on neutral alumina eluting with 1% ethyl acetate in hexane to give (2S)-4-benzyl-2-[(benzyloxy)methyl]-5,5-dimethyl-1,4-oxazepane (2.0 g, 5.9 mmol, 24% yield) as a yellow liquid. 1H NMR (400 MHz, DMSO-d6) δ 7.42-7.34 (m, 2H), 7.33-7.17 (m, 6H), 7.04-6.96 (m, 2H), 4.23-4.05 (m, 2H), 3.87 (d, 1H), 3.79 (ddd, 1H), 3.71 (ddd, 1H), 3.45-3.35 (m, 1H), 3.35-3.32 (m, 2H), 3.23 (dd, 1H), 3.00 (dd, 1H), 2.69 (dd, 1H), 1.85-1.70 (m, 2H), 1.17 (s, 3H), 1.06 (s, 3H). LCMS (Method 4-Column 1): Retention Time=3.18 minutes, [MH]+=341.

### Synthesis of tert-butyl (2S)-2-(hydroxymethyl)-5,5-dimethyl-1,4-oxazepane-4-carboxylate

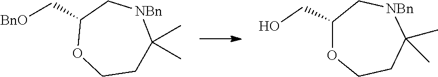 [SMILES: CC1(C)CCO[C@H](COCc2ccccc2)CN1Cc1ccccc1 ; CC1(C)CCO[C@H](CO)CN1Cc1ccccc1]

To a solution of (2S)-4-benzyl-2-[(benzyloxy)methyl]-5,5-dimethyl-1,4-oxazepane in ethanol (10 mL) were added palladium on carbon (10% w/w, 2.0 g, 1.9 mmol) and di-tert-butyl decarbonate (1.6 g, 7.6 mmol). The reaction mixture was placed in a hydrogenator and stirred at room temperature for 96 hours under 200 psi of hydrogen. After this time the reaction mixture was filtered through Celite®, washing with methanol, the filtrate was concentrated under reduced pressure and the crude material was purified by column chromatography on silica gel eluting with 25% ethyl acetate in hexane to give tert-butyl (2S)-2-(hydroxymethyl)-5,5-dimethyl-1,4-oxazepane-4-carboxylate (900 mg, 3.5 mmol, 59% yield) as a pale yellow liquid. 1H NMR (400 MHz, DMSO-d6) δ 4.65 (t, 1H), 3.92 (d, 1H), 3.82 (ddd, 1H), 3.60 (dd, 1H), 3.44-3.37 (m, 1H), 3.29-3.12 (m, 2H), 3.04 (dd, 1H), 2.01 (dd, 1H), 1.72 (dd, 1H), 1.44 (s, 3H), 1.39 (s, 9H), 1.28 (s, 3H). LCMS (Method 4-Column 1): Retention Time=1.73 minutes, [(M−100)H]+=160. HPLC: Retention Time=6.72 minutes.

### Synthesis of ethyl 4-benzyl-2-methylmorpholine-2-carboxylate

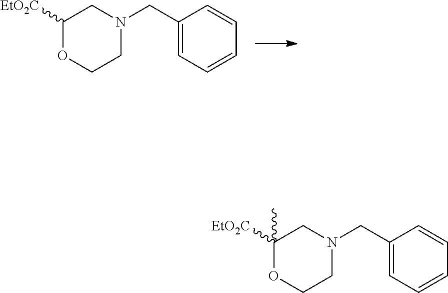

To a solution of ethyl 4-benzylmorpholine-2-carboxylate (4.8 g, 19.3 mmol) in tetrahydrofuran (50 mL) cooled to −78° C. under nitrogen was added a lithium diisopropylamide (2.0 M solution in THF/heptane/ethylbenzene, 14.4 mL, 28.9 mmol) and the reaction was 30 minutes before addition of iodomethane (1.2 mL, 19.3 mmol). The reaction was stirred at −78° C. for a further 1 hour and was quenched by addition of a saturated aqueous ammonium chloride solution. The products were extracted with ethyl acetate. The combined organic layers were washed with brine, dried over anhydrous sodium sulfate and concentrated. The crude material was purified by column chromatography on silica gel eluting with 5-10% ethyl acetate in petroleum ether to give ethyl 4-benzyl-2-methylmorpholine-2-carboxylate (3.6 g, 13.7 mmol, 71% yield) as an oil. 1H NMR (400 MHz, Chloroform-d) δ 7.32-7.24 (m, 5H), 4.23 (qd, 2H), 4.03 (td, 1H), 3.77 (ddd, 1H), 3.55 (d, 1H), 3.40 (d, 1H), 3.23 (dd, 1H), 2.66-2.57 (m, 1H), 2.24 (td, 1H), 1.98 (d, 1H), 1.32 (s, 3H), 1.27 (t, 3H). LCMS (Method 5): Retention Time=1.91 minutes, [MH]+=264.

### Synthesis of [(2S)-4-benzyl-2-methylmorpholin-2-yl]methanol and [(2R)-4-benzyl-2-methylmorpholin-2-yl]methanol

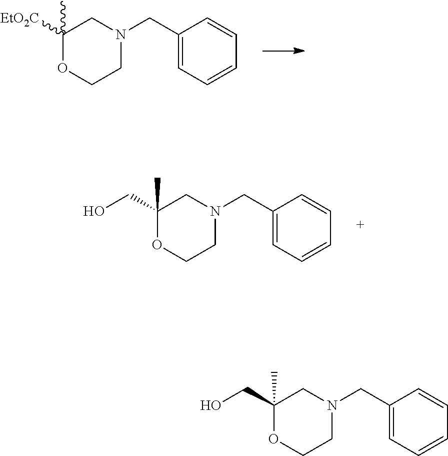

To a solution of ethyl 4-benzyl-2-methylmorpholine-2-carboxylate (3.6 g, 13.7 mmol) in ethanol (50 mL) cooled to 0° C. was added sodium borohydride (1.0 g, 27.3 mmol). The reaction was stirred at room temperature for 16 hours. The reaction mixture was quenched by addition of ice and the products were extracted with ethyl acetate. The combined organic phases were washed with brine, dried over anhydrous sodium sulfate and concentrated. The crude material was purified by column chromatography on silica gel eluting with 20-50% ethyl acetate in petroleum ether gradient to give the title compounds as a mixture of enantiomers. The enantiomers were separated by chiral supercritical fluid chromatography (LUX A1 (250×30 mm), 5 micron column; mobile phase: CO₂: methanol (90:10)) to give: enantiomer 1: [(2S)-4-benzyl-2-methylmorpholin-2-yl]methanol (1.1 g, 4.7 mmol, 38% yield) and enantiomer 2: [(2R)-4-benzyl-2-methylmorpholin-2-yl]methanol (1.0 g, 4.5 mmol, 36% yield). The absolute configurations were not assigned.

### Enantiomer 1: [(2S)-4-benzyl-2-methylmorpholin-2-yl]methanol

1H NMR (400 MHz, DMSO-d6) δ 7.36-7.28 (m, 4H), 7.27-7.19 (m, 1H), 4.58 (t, 1H), 3.70-3.56 (m, 2H), 3.46 (d, 1H), 3.41-3.30 (m, 3H), 2.42-2.32 (m, 1H), 2.28-2.12 (m, 3H), 1.15 (s, 3H). LCMS (Method 6): Retention Time=3.90 minutes, [MH]+=222. SFC: Retention Time=3.76 minutes, 99% ee.

### Enantiomer 2: [(2R)-4-benzyl-2-methylmorpholin-2-yl]methanol

1H NMR (400 MHz, DMSO-d6) δ 7.42-7.07 (m, 5H), 4.57 (t, 1H), 3.70-3.55 (m, 2H), 3.47 (d. 1H), 3.39-3.27 (m, 3H), 2.42-2.33 (m, 1H), 2.28-2.10 (m, 3H), 1.13 (s, 3H). LCMS (Method 6): Retention Time=2.22 minutes, [MH]+=222. SFC: Retention Time=4.35 minutes, 99% ee.

### Synthesis of [(2S)-2-methylmorpholin-2-yl]methanol

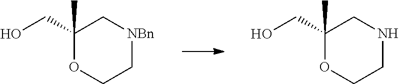

To a solution of [(2S)-4-benzyl-2-methylmorpholin-2-yl]methanol (1.0 g, 4.5 mmol) in methanol (20 mL) was added palladium(II) hydroxide (20% loading wet support, 317 mg, 0.22 mmol) under nitrogen. The reaction mixture was sealed, hydrogen bladder attached and stirring was continued for 6 hours. After this time, the reaction mixture was filtered through a pad of Celite® washed with methanol and the filtrate was concentrated under reduced pressure. The resulting crude product [(2S)-2-methylmorpholin-2-yl]methanol (590 mg 4.5 mmol, 100% yield) was taken for the next step without purification. 1H NMR (400 MHz, Methanol-d4) δ 3.77-3.64 (m, 2H), 3.54 (d, 1H), 3.46 (d, 1H), 2.84 (d, 1H), 2.80-2.72 (m, 2H), 2.62 (d, 1H), 1.20 (s, 3H). LCMS (Method 5): Retention Time=0.52 minutes, [MH]+=132.

### Synthesis of [(2R)-2-methylmorpholin-2-yl]methanol

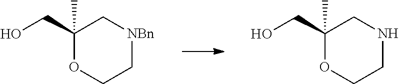

[(2R)-4-benzyl-2-methylmorpholin-2-yl]methanol (1.0 g, 4.52 mmol) was de-benzylated under analogous conditions to isomer 1 to give [(2R)-2-methylmorpholin-2-yl]methanol (600 mg, 4.6 mmol, 101% yield). The crude material was taken to the next step without purification 1H NMR (400 MHz, Methanol-d4) δ 3.77-3.65 (m, 2H), 3.54 (d, 1H), 3.46 (d, 1H), 2.84 (d, 1H), 2.79-2.72 (m, 2H), 2.62 (d, 1H), 1.20 (s, 3H). LCMS (Method 5): Retention Time=0.71 minutes, [MH]+=132.

### Synthesis of tert-butyl (2S)-2-(hydroxymethyl)-2-methylmorpholine-4-carboxylate

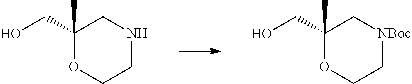

To a solution of [(2S)-2-methylmorpholin-2-yl]methanol (600 mg, 4.6 mmol) in dichloromethane (10 mL) and water (10 mL) was added a solution of sodium hydroxide (4 M, 183 mg, 4.6 mmol), followed by a solution of di-tert-butyl dicarbonate (1.0 g, 4.8 mmol) in dichloromethane (10 mL) and the reaction was stirred at room temperature for 2 hours. The reaction mixture was partitioned between water and dichloromethane and the aqueous layer was extracted with dichloromethane. The combined organic layers were washed with brine, dried over anhydrous sodium sulfate and concentrated. The crude material was purified by column chromatography on silica gel eluting with a gradient of ethyl acetate in petroleum ether to give (2S)-2-(hydroxymethyl)-2-methylmorpholine-4-carboxylate (400 mg, 1.7 mmol, 38% yield) as a colourless oil. 1H NMR (400 MHz, DMSO-d6) δ 4.71 (t, 1H), 3.63-3.50 (m, 2H), 3.48-3.40 (m, 1H), 3.31-3.22 (m, 3H), 3.17-2.99 (m, 2H), 1.40 (s, 9H), 1.05 (s, 3H). LCMS (Method 5): Retention Time=2.23 minutes, [(M−100)H]+=132.

### Synthesis of tert-butyl (2R)-2-(hydroxymethyl)-2-methylmorpholine-4-carboxylate

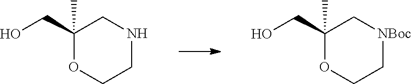

[(2R)-2-methylmorpholin-2-yl]methanol (600 mg, 4.6 mmol) was subjected to analogous procedure as [(2S)-2-methylmorpholin-2-yl]methanol (isomer 1) to give tert-butyl (2R)-2-(hydroxymethyl)-2-methylmorpholine-4-carboxylate (400 mg, 1.7 mmol, 38% yield). 1H NMR (400 MHz, DMSO-d6) δ 4.72 (t, 1H), 3.64-3.50 (m, 2H), 3.49-3.38 (m, 1H), 3.31-3.22 (m, 3H), 3.19-2.98 (m, 2H), 1.40 (s, 9H), 1.05 (s, 3H). LCMS (Method 5): Retention Time=2.23 minutes, [(M−100)H]+=132.

### Synthesis of 1-[(4R)-2,2-dimethyl-1,3-dioxolan-4-yl]ethan-1-one

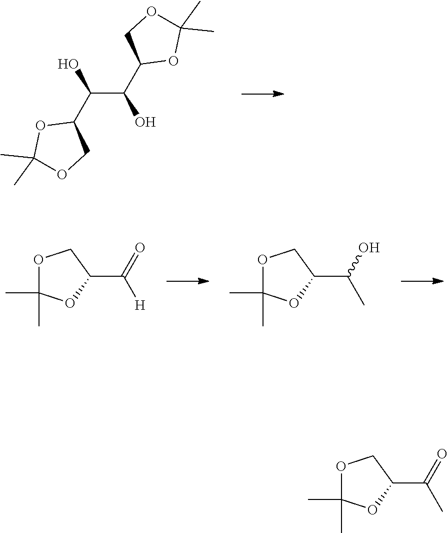

To a solution of (1S,2S)-1,2-bis[(4R)-2,2-dimethyl-1,3-dioxolan-4-yl]ethane-1,2-diol (40.0 g, 152.5 mmol) in dichloromethane (200 mL) warmed to 30° C. was added 10% aqueous sodium bicarbonate solution (20 mL) followed by portion-wise addition of sodium metaperiodate (48.9 g, 228.8 mmol). The reaction mixture was stirred at 30° C. for 2 hours. After this time, the reaction mixture was cooled to room temperature and anhydrous magnesium sulfate was added and stirred for 10 minutes. The reaction mixture was filtered and solvents removed under reduced pressure to give (4R)-2,2-dimethyl-1,3-dioxolane-4-carbaldehyde (31.8 g, 244.4 mmol, 160.2% yield). The material was taken through to the next step without purification.

To a solution of crude (4R)-2,2-dimethyl-1,3-dioxolane-4-carbaldehyde (31.8 g, 244.4 mmol) in tetrahydrofuran (100 mL) cooled to −78° C. was added dropwise methylmagnesium bromide (3 M in diethyl ether, 122 mL, 244.3 mmol). The reaction mixture was stirred for a further 30 minutes at −78° C. before warming to room temperature and stirring was continued for 2 hours. The reaction mixture was cooled to 0° C., quenched by dropwise addition of saturated aqueous ammonium chloride solution and the products were extracted with ethyl acetate. The combined organic layers were washed with water, dried over anhydrous sodium sulfate and distilled to give 1-[(4R)-2,2-dimethyl-1,3-dioxolan-4-yl]ethan-1-ol (28.3 g, 193.6 mmol, 79% yield, collected at 30° C. at 150 mbar). The material was taken through to the next step without purification.

To a solution of dimethyl sulfoxide (71 mL, 997.0 mmol) in dichloromethane (250 mL) cooled to −78° C. were added sequentially oxalyl chloride (34 mL, 396.9 mmol), (1-[(4R)-2,2-dimethyl-1,3-dioxolan-4-yl]ethanol (28.3 g, 193.6 mmol) and triethylamine (283 mL, 2.0 mol) and the reaction mixture was warmed to −20° C. and stirred at this temperature for 1 hour. After this time, the reaction mixture was quenched with water and extracted with dichloromethane. The combined organic layers were washed with water, dried over anhydrous sodium sulfate and distilled at 25° C. at 150 mbar to give 1-[(4R)-2,2-dimethyl-1,3-dioxolan-4-yl]ethan-1-one (36.6 g, 253.9 mmol, 131% yield). The material was taken through to the next step without purification. 1H NMR (400 MHz, Chloroform-d) δ 4.40 (dd, 1H), 4.19 (dd, 1H), 3.99 (dd, 1H), 2.25 (s, 3H), 1.48 (s, 3H), 1.39 (s, 3H).

### Synthesis of benzyl[(rel-1R)-1-[(4S)-2,2-dimethyl-1,3-dioxolan-4-yl]ethyl]amine

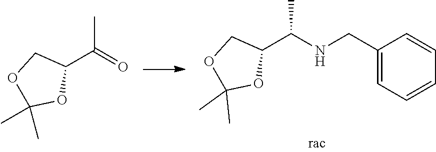

To a solution of (4R)-2,2-dimethyl-1,3-dioxolane-4-carbaldehyde (36.6 g, 253.9 mmol) and benzylamine (32.6 g, 304.6 mmol) in 1,2-dichloroethane (200 mL) was added acetic acid (1.4 mL, 25.4 mmol) and the reaction mixture was cooled to 0° C. Sodium triacetoxyborohydride (80.7 g, 380.8 mmol) was added portion-wise and the reaction mixture was allowed to warm to room temperature and it was stirred for 16 hours. The reaction mixture was poured into an ice-cold saturated aqueous solution of sodium bicarbonate and the products were extracted with dichloromethane. The combined organic phases were washed with water, dried over anhydrous sodium sulfate and concentrated under reduced pressure. The crude residue was purified by column chromatography on silica gel eluting with 30% ethyl acetate in petroleum ether to give benzyl[rel-(1R)-1-[(4S)-2,2-dimethyl-1,3-dioxolan-4-yl]ethyl]amine (14.6 g, 62.0 mmol, 24% yield) as a pale yellow oil. 1H NMR (400 MHz, Chloroform-d) δ 7.35-7.29 (m, 4H), 7.27-7.21 (m, 1H), 4.08-3.96 (m, 2H), 3.94-3.85 (m, 2H), 3.75 (d, 1H), 2.89-2.78 (m, 1H), 1.41 (d, 3H), 1.35 (d, 3H), 1.10 (d, 3H). LCMS (Method 7): Retention Time=3.00 minutes, [MH]+=236.

### Synthesis of rel-(2S,3R)-3-(benzylamino)butane-1,2-diol

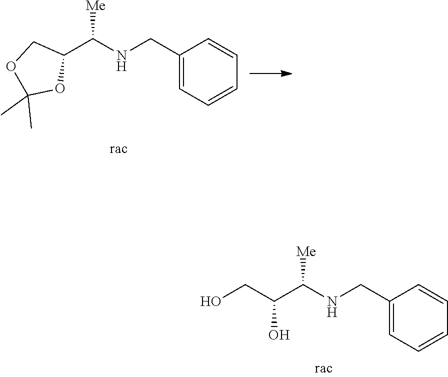 [SMILES: C[C@H](NCc1ccccc1)[C@H]1COC(C)(C)O1 ; C[C@H](NCc1ccccc1)[C@H](O)CO]

To a solution of benzyl[rel-(1R)-1-[(4S)-2,2-dimethyl-1,3-dioxolan-4-yl]ethyl]amine (14.6 g, 62 mmol) in acetone (20 mL) was added p-toluenesulfonic acid monohydrate (23.6 g, 124.1 mmol) and water (40 mL) and the reaction mixture was heated to 80° C. for 3 hours. The cooled reaction mixture was neutralised with aqueous 10% sodium hydroxide and the products were extracted with dichloromethane. The combined organic layers were concentrated under reduced pressure to give rel-(2S,3R)-3-(benzylamino)butane-1,2-diol (10.5 g, 53.6 mmol, 86% yield). The material was taken through to the next step without purification. 1H NMR (300 MHz, DMSO-d6) δ 7.40-7.26 (m, 4H), 7.26-7.16 (m, 1H), 4.64 (br s, 1H), 4.46 (d, 1H), 3.77 (d, 1H), 3.66 (d, 1H), 3.53-3.41 (m, 1H), 3.41-3.26 (m, 2H), 2.70-2.54 (m, 1H), 1.99-1.68 (m, 1H), 0.97 (d, 3H). LCMS (Method 6): Retention Time=2.69 minutes, [MH]+=196.

### Synthesis of rel-(2S,3R)-3-(benzylamino)-1-[(tert-butyldimethylsilyl)oxy]butan-2-ol

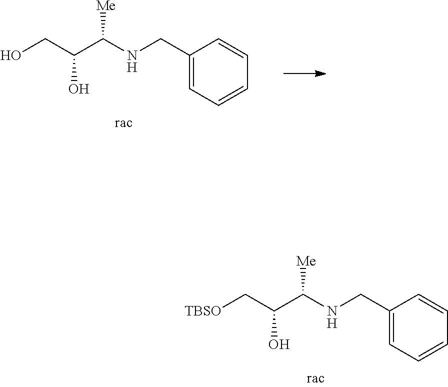 [SMILES: C[C@H](NCc1ccccc1)[C@H](O)CO ; C[C@H](NCc1ccccc1)[C@H](O)CO[Si](C)(C)C(C)(C)C]

To a solution of rel-(2S,3R)-3-(benzylamino)butane-1,2-diol (10.4 g, 53.3 mmol) in dichloromethane (50 mL) cooled to 0° C. was added 1H-imidazole (10.9 g, 159.8 mmol) and tert-butyldimethylchlorosilane (8.0 g, 53.3 mmol). The reaction mixture was stirred at 0° C. for 2 hours. The reaction mixture was poured into water and the products extracted with dichloromethane. The combined organic layers were washed with water, dried over anhydrous sodium sulfate and concentrated. The crude material was purified by column chromatography on silica gel eluting with 4% methanol in dichloromethane to give rel-(2S,3R)-3-(benzylamino)-1-[(tert-butyldimethylsilyl)oxy]butan-2-ol (12.8 g, 41.4 mmol, 78% yield) as a colourless oil. 1H NMR (300 MHz, DMSO-d6) δ 7.43-6.98 (m, 5H), 4.47 (br s, 1H), 3.75 (d, 1H), 3.67 (d, 1H), 3.33 (s, 1H), 2.78-2.58 (m, 1H), 1.85 (br s, 1H), 0.94 (d, 3H), 0.82 (s, 9H), 0.01 (s, 6H). LCMS (Method 5): Retention Time=2.32 minutes, [MH]=310.

### Synthesis of N-benzyl-N-[rel-(2R,3S)-4-[(tert-butyldimethylsilyl)oxy]-3-hydroxybutan-2-yl]-2-chloroacetamide

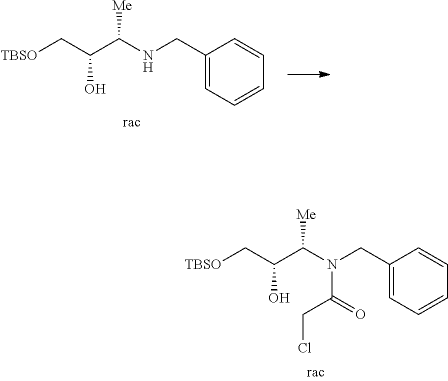 [SMILES: C[C@H](NCc1ccccc1)[C@H](O)CO[Si](C)(C)C(C)(C)C ; C[C@@H]([C@H](O)CO[Si](C)(C)C(C)(C)C)N(Cc1ccccc1)C(=O)CCl]

To a solution of rel-(2S,3R)-3-(benzylamino)-1-[(tert-butyldimethylsilyl)oxy]butan-2-ol (12.8 g, 41.4 mmol) and triethylamine (8.4 g, 82.7 mmol) in dichloromethane (50 mL) cooled to 0° C. was added dropwise chloroacetyl chloride (5.6 g, 49.6 mmol) and the reaction was stirred at this temperature for 30 minutes and then at room temperature for 16 hours. The reaction mixture was poured into water and the products extracted with dichloromethane. The combined organic layers were washed with water, dried over anhydrous sodium sulfate and concentrate. The crude material was purified by column chromatography on silica gel eluting with 9% ethyl acetate in petroleum ether to give N-benzyl-N-[rel-(2S,3S)-4-[(tert-butyldimethylsilyl)oxy]-3-hydroxybutan-2-yl]-2-chloroacetamide (11.6 g, 30.1 mmol, 73% yield) as a colourless oil. 1H NMR (400 MHz, DMSO-d6)(Mixture of rotamers 2:1) 67.51-7.09 (m, 5H), 5.05 (d, 0.6H), 4.87 (d, 0.3H), 4.78-4.57 (m, 2H), 4.45-4.30 (m, 1H), 4.27-3.95 (m, 1H), 3.82-3.72 (m, 0.3H), 3.70-3.59 (m, 0.6H), 3.51-3.37 (m, 2H), 1.10 (d, 2H), 1.04 (d, 1H), 0.87 (s, 9H), 0.07-0.03 (m, 6H). LCMS (Method 5): Retention Time=3.55 minutes, [MH]+=386.

### Synthesis of rel-(5S,6S)-4-benzyl-6-{[(tert-butyldimethylsilyl)oxy]methyl}-5-methylmorpholin-3-one

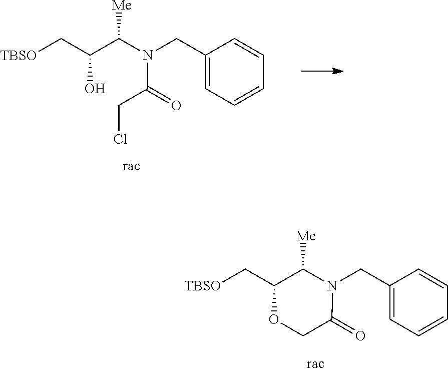 [SMILES: C[C@@H]([C@H](O)CO[Si](C)(C)C(C)(C)C)N(Cc1ccccc1)C(=O)CCl ; C[C@H]1[C@@H](CO[Si](C)(C)C(C)(C)C)OCC(=O)N1Cc1ccccc1]

To a solution of N-benzyl-N-[rel-(2S,3S)-4-[(tert-butyldimethylsilyl)oxy]-3-hydroxybutan-2-yl]-2-chloroacetamide (11.6 g, 30.1 mmol) in tert-butanol (100 mL) was added potassium tert-butoxide (3.5 g, 31.6 mmol) and the reaction mixture was stirred at room temperature for 3 hours. The reaction mixture was poured into a saturated aqueous solution of ammonium chloride and the products were extracted with ethyl acetate. The combined organic phases were washed with water, dried over sodium sulfate and concentrated. The crude residue was purified by column chromatography on silica gel eluting with 5% ethyl acetate in petroleum ether to give rel-(5S,6S)-4-benzyl-6-{[(tert-butyldimethylsilyl)oxy]methyl}-5-methylmorpholin-3-one (8.5 g, 24.3 mmol, 81% yield) as a pale yellow oil. 1H NMR (400 MHz, Chloroform-d) δ 7.36-7.21 (m, 5H), 5.39 (d, 1H), 4.33 (dd, 1H), 4.26 (d, 1H), 3.91 (d, 1H), 3.75-3.63 (m, 2H), 3.45 (dd, 1H), 3.35 (qd, 1H), 1.17 (d, 3H), 0.78 (s, 9H), 0.00 (s, 3H), −0.03 (s, 3H). LCMS (Method 5): Retention Time=3.66 minutes, [MH]+=350.

### Synthesis of rel-(2S,3S)-4-benzyl-2-{[(tert-butyldimethylsilyl)oxy]methyl}-3-methylmorpholine

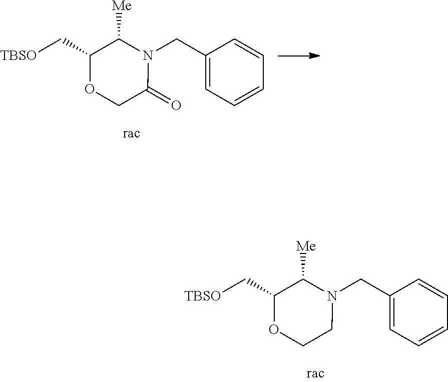 [SMILES: C[C@H]1[C@@H](CO[Si](C)(C)C(C)(C)C)OCC(=O)N1Cc1ccccc1 ; C[C@H]1[C@@H](CO[Si](C)(C)C(C)(C)C)OCCN1Cc1ccccc1]

To a solution of rel-(5S,6S)-4-benzyl-6-{[(tert-butyldimethylsilyl)oxy]methyl}-5-methylmorpholin-3-one (8.5 g, 24.3 mmol) in tetrahydrofuran (30 mL) cooled to 0° C. was added dropwise borane dimethylsulfide (4.6 mL, 48.6 mmol). After stirring at 0° C. for 30 minutes the reaction mixture was warmed to room temperature and stirred for 2 hours. The reaction was cooled to 0° C. and methanol (50 ml) was added dropwise and then heated to reflux for 30 minutes. On completion, the reaction mixture was cooled to room temperature and concentrated. The crude residue was purified by column chromatography on silica gel eluting with 2% ethyl acetate in petroleum ether to give rel-(2S,3S)-4-benzyl-2-{[(tert-butyldimethylsilyl)oxy]methyl}-3-methylmorpholine (7.5 g, 22.4 mmol, 92% yield) as a colourless oil. 1H NMR (400 MHz, Chloroform-d) δ 7.38-7.33 (m, 2H), 7.33-7.27 (m, 2H), 7.26-7.20 (m, 1H), 3.83-3.72 (m, 2H), 3.70-3.50 (m, 4H), 3.44 (dd, 1H), 2.92 (qd, 1H), 2.65 (td, 1H), 2.29 (dd, 1H), 0.93 (d, 3H), 0.85 (s, 9H), 0.05 (s, 6H). LCMS (Method 5): Retention Time=2.44 minutes, [MH]+=336.

### Synthesis of [(2S,3S)-4-benzyl-3-methylmorpholin-2-yl]methanol and [(2R,3S)-4-benzyl-3-methylmorpholin-2-yl]methanol

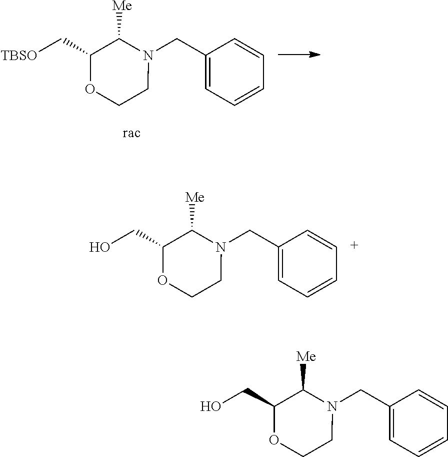 [SMILES: C[C@H]1[C@@H](CO[Si](C)(C)C(C)(C)C)OCCN1Cc1ccccc1 ; C[C@H]1[C@@H](CO)OCCN1Cc1ccccc1 ; C[C@@H]1[C@H](CO)OCCN1Cc1ccccc1]

To a solution of rel-(2S,3S)-4-benzyl-2-([(tert-butyldimethylsilyl)oxy]methyl)-3-methylmorpholine (7.4 g, 22.1 mmol) in tetrahydrofuran (75 mL) cooled to 0° C., was added a solution of tetra-n-butylammonium fluoride (1 M in tetrahydrofuran, 33 mL, 33.1 mmol). The reaction mixture stirred at room temperature for 2 hours. The reaction mixture was poured into water and the products were extracted with dichloromethane. The combined organic layers were washed with water, dried over anhydrous sodium sulfate and concentrated. The crude product was purified by flash chromatography on silica gel eluting with 27% ethyl acetate in petroleum ether to give the desired product as a racemic mixture (4.3 g) as a red oil. The enantiomers were separated by chiral supercritical fluid chromatography (LUX A1 (250×30 mm) 5 micron column; mobile phase: CO₂: 0.5% isopropylamine in isopropyl alcohol (60:40) to give [(2R,3R)-4-benzyl-3-methylmorpholin-2-yl]methanol (420 mg, 1.9 mmol, 9% yield) and [(2S,3S)-4-benzyl-3-methylmorpholin-2-yl]methanol (2.8 g, 12.7 mmol, 57% yield) as red oils.

### Isomer 1:

1H NMR (400 MHz, Chloroform-d) δ 7.36-7.29 (m, 4H), 7.27-7.22 (m, 1H), 3.91 (dt, 1H), 3.78 (dt, 1H), 3.75-3.62 (m, 3H), 3.57-3.47 (m, 2H), 2.83 (qd, 1H), 2.68 (ddd, 1H), 2.32 (dt, 1H), 0.98 (d, 3H). LCMS (Method 6): Retention Time=2.59 minutes, [MH]+=222.

### Isomer 2:

1H NMR (400 MHz, Chloroform-d) δ 7.37-7.28 (m, 4H), 7.27-7.20 (m, 1H), 3.91 (dt, 1H), 3.77 (dt, 1H), 3.74-3.64 (m, 3H), 3.56-3.44 (m, 2H), 2.83 (qd, 1H), 2.68 (ddd, 1H), 2.32 (dt, 1H), 0.98 (d, 3H). LCMS (Method 6): Retention Time=2.59 minutes, [MH]+=222.

### Synthesis of [(2S,3S)-3-methylmorpholin-2-yl]methanol

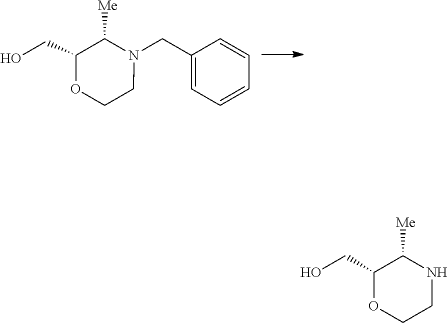 [SMILES: C[C@H]1[C@@H](CO)OCCN1Cc1ccccc1 ; C[C@@H]1NCCO[C@@H]1CO]

To a degassed solution of [(2S,3S)-4-benzyl-3-methylmorpholin-2-yl]methanol (2.7 g, 12.2 mmol) in methanol (60 mL) was added palladium(II) hydroxide (20% loading wet support, 550 mg, 0.39 mmol) and the reaction was stirred at room temperature for 16 hours. After this time, the reaction mixture was filtered through Celite® washing with methanol and the filtrate was concentrated to give [(2S,3S)-3-methylmorpholin-2-yl]methanol (1.5 g, 11.4 mmol, 94% yield) as a colourless oil. 1H NMR (300 MHz, DMSO-d6) δ 4.56 (br s, 1H), 3.68-3.65 (m, 1H), 3.49-3.48 (m, 1H), 3.47-3.37 (m, 2H), 3.25-3.17 (m, 2H), 2.87-2.83 (m, 2H), 0.97 (d, 3H). LCMS (Method 6): Retention Time=0.94 minutes, [MH]+=132.

### Synthesis of tert-butyl (2S,3S)-2-(hydroxymethyl)-3-methylmorpholine-4-carboxylate

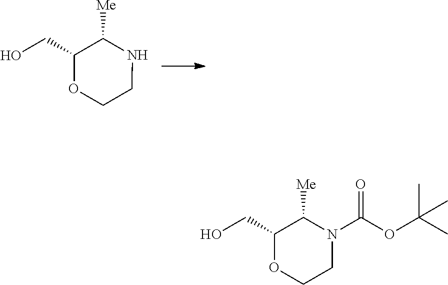 [SMILES: C[C@@H]1NCCO[C@@H]1CO ; C[C@H]1[C@@H](CO)OCCN1C(=O)OC(C)(C)C]

To a solution of [(2S,3S)-3-methylmorpholin-2-yl]methanol (1.3 g, 9.9 mmol) in dichloromethane (60 mL) was added triethyl amine (1.4 g, 13.6 mmol) followed by di-tert-butyl dicarbonate (2.2 g, 10.1 mmol) and the reaction was stirred at room temperature for 3 hours. After this time, the reaction mixture was poured into water and the products were extracted with dichloromethane. The combined organic layers were washed with water, dried over sodium sulfate and concentrated. The crude material was purified by column chromatography on silica gel eluting with 23% ethyl acetate in petroleum ether to give tert-butyl (2S,3S)-2-(hydroxymethyl)-3-methylmorpholine-4-carboxylate (1.9 g, 8.2 mmol, 83% yield) as a colourless oil. 1H NMR (300 MHz, DMSO-d6) δ 4.75 (br s, 1H), 4.03-3.94 (m, 1H), 3.83-3.76 (m, 1H), 3.58-3.52 (m, 1H), 3.41-3.34 (m, 3H), 3.28-3.19 (m, 1H), 3.03-2.92 (m, 1H), 1.40 (s, 9H), 0.99-0.97 (m, 3H). LCMS (Method 6): Retention Time=1.45 minutes, [(M-100)H]+=132.

### Synthesis of 3-bromo-1-tert-butyl-1H-pyrrole

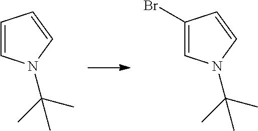

To a solution of 1-tert-butyl-1H-pyrrole (2.0 g, 16.2 mmol) in tetrahydrofuran (30 mL) cooled to −78° C. was added N-bromosuccinimide (2.3 g, 13.0 mmol). After 2 hours, the reaction mixture was treated with water (100 mL) and the products were extracted with ethyl acetate. The combined organic layers were dried over anhydrous sodium sulfate and concentrated. The crude material was purified by column chromatography on silica gel eluting with 2% ethyl acetate in n-hexane to give 3-bromo-1-tert-butyl-1H-pyrrole (2.1 g, 10.4 mmol, 64% yield) as a colourless oil. 1H NMR (400 MHz, Chloroform-d) δ 6.80 (dd, 1H), 6.73 (app t, 1H), 6.14 (dd, 1H), 1.50 (s, 9H). LCMS (Method 4-Column 5): Retention Time=2.47 minutes, [MH]+=202/204.

### Synthesis of 1-tert-butyl-3-(4,4,5,5-tetramethyl-1,3,2-dioxaborolan-2-yl)-1H-pyrrole

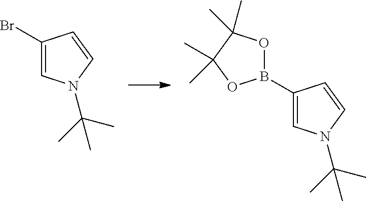

To a solution of 3-bromo-1-tert-butylpyrrole (2.0 g, 9.9 mmol) in toluene (3 mL) was added 4,4,5,5-tetramethyl-1,3,2-dioxaborolane (1.5 g, 11.9 mmol), bis(acetonitrile)dichloropalladium(II) (77 mg, 0.03 mmol), 2-dicyclohexylphosphino-2′,6′-dimethoxybiphenyl (365 mg, 0.09 mmol) and triethylamine (3.5 mL, 24.7 mmol). The reaction was heated to 80° C. for 6 hours. The reaction mixture was partitioned between water and ethyl acetate and the products were extracted with ethyl acetate. The combined organic layers were dried over anhydrous sodium sulfate and concentrated. The crude material was purified by column chromatography on silica gel eluting with 2% ethyl acetate in n-hexane to give 1-tert-butyl-3-(4,4,5,5-tetramethyl-1,3,2-dioxaborolan-2-yl)-1H-pyrrole (0.9 g, 3.6 mmol, 37% yield) as a pale-yellow solid. 1H NMR (400 MHz, DMSO-d6) δ 7.17 (s, 1H), 7.01-6.86 (m, 1H), 6.19 (app t, 1H), 1.46 (s, 9H), 1.22 (s, 12H). LCMS (Method 4-Column 2): Retention Time=2.43 minutes, [MH]+=250.

### Synthesis of 3-[2-(2,2,2-trichloroacetyl)-1H-pyrrol-1-yl]propanenitrile

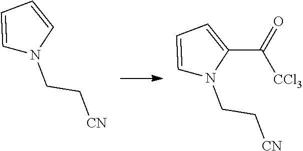

To a solution of 3-(1H-pyrrol-1-yl)propanenitrile (5.0 g, 41.6 mmol) in dichloromethane (100 mL) cooled to 0° C. was added trichloroacetyl chloride (5.7 mL, 49.9 mmol) and the reaction was stirred for 2 hours. The reaction mixture was treated with a mixture of ice and water and the products were extracted with dichloromethane. The combined organic extracts were washed with a saturated aqueous sodium bicarbonate solution and brine, dried over anhydrous sodium sulfate and concentrated. The residue was purified by column chromatography on silica gel eluting with 10-20% ethyl acetate in petroleum ether gradient to give 3-[2-(2,2,2-trichloroacetyl)-1H-pyrrol-1-yl]propanenitrile (4.3 g, 16.2 mmol, 39% yield) as a colourless solid. 1H NMR (400 MHz, Chloroform-d) δ 7.65-7.63 (m, 1H), 7.20-7.18 (m, 1H), 6.37-6.35 (m, 1H), 4.60 (t, 2H), 2.94 (t, 2H).

### Synthesis of 3-[4-bromo-2-(2,2,2-trichloroacetyl)-1H-pyrrol-1-yl]propanenitrile

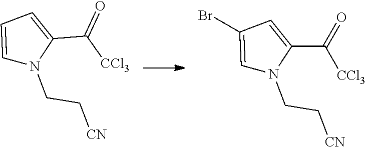

To a solution of 3-[2-(2,2,2-trichloroacetyl)-1H-pyrrol-1-yl]propanenitrile (5.0 g, 18.8 mmol) in dichloromethane (500 mL) cooled to −20° C. was added N-bromosuccinimide (3.4 g, 18.8 mmol) and the reaction mixture was stirred at room temperature for 2 hours. A mixture of ice and water was added, and the products were extracted with ethyl acetate. The combined organic layers were washed with water and brine, dried over anhydrous sodium sulfate and concentrated. The crude material was purified by column chromatography on silica gel eluting with 10-20% ethyl acetate in petroleum ether gradient to give 3-[4-bromo-2-(2,2,2-trichloroacetyl)-1H-pyrrol-1-yl]propanenitrile (4.6 g, 13.4 mmol, 71% yield) as a colourless solid. 1H NMR (400 MHz, DMSO-d6): δ 7.79 (s, 1H), 7.52 (s, 1H), 4.59 (t, 2H), 3.04 (t, 2H).

### Synthesis of methyl 4-bromo-1-(2-cyanoethyl)-1H-pyrrole-2-carboxylate

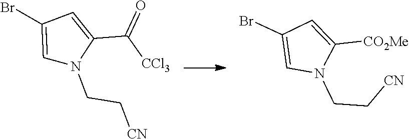

To a solution of 3-[4-bromo-2-(2,2,2-trichloroacetyl)-1H-pyrrol-1-yl]propanenitrile (5.2 g, 15.1 mmol) in methanol (100 mL) was added potassium carbonate (4.2 g, 15.1 mmol) and the reaction was stirred at room temperature for 2 hours. A mixture of ice and water was added, and the products were extracted with ethyl acetate. The combined organic layers were washed with water and brine, then dried over anhydrous sodium sulfate and concentrated. The crude material was purified by column chromatography on silica gel eluting with 5-15% ethyl acetate in petroleum ether gradient to give methyl 4-bromo-1-(2-cyanoethyl)-1H-pyrrole-2-carboxylate (3.6 g, 14.0 mmol, 93% yield) as a colourless solid. 1H NMR (400 MHz, DMSO-d6): δ 7.44 (s, 1H), 6.94 (s, 1H), 4.54 (t, 2H), 3.76 (s, 3H), 3.02 (t, 2H).

### Synthesis of methyl 4-bromo-1-(3-methoxy-3-oxopropyl)-1H-pyrrole-2-carboxylate

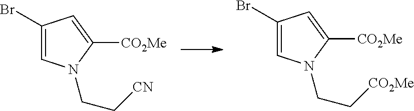 [SMILES: COC(=O)c1cc(Br)cn1CCC#N ; COC(=O)CCn1cc(Br)cc1C(=O)OC]

A solution of methyl 4-bromo-1-(2-cyanoethyl)-1H-pyrrole-2-carboxylate (3.5 g, 13.6 mmol) in a mixture (1:5) of sulfuric acid (15 mL, 13.6 mmol) and methanol (75 mL) was heated to 80° C. for 5 hours in a sealed vessel. A mixture of ice and water was added and the products were extracted with ethyl acetate. The combined organic layers were washed with brine, dried over anhydrous sodium sulfate and concentrated. The crude material was purified by column chromatography on silica gel eluting with 5-10% ethyl acetate in petroleum ether gradient to give methyl 4-bromo-1-(3-methoxy-3-oxopropyl)-1H-pyrrole-2-carboxylate (2.3 g, 7.9 mmol, 58% yield) as a liquid. 1H NMR (400 MHz, DMSO-d6): δ 7.33 (s, 1H), 6.88 (s, 1H), 4.50 (t, 2H), 3.75 (s, 3H), 3.59 (s, 3H), 2.80 (t, 2H).

### Synthesis of 4-bromo-1-(2-carboxyethyl)-1H-pyrrole-2-carboxylic acid

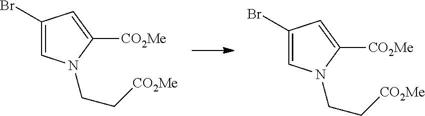 [SMILES: COC(=O)CCn1cc(Br)cc1C(=O)OC ; COC(=O)CCn1cc(Br)cc1C(=O)OC]

To a solution of methyl 4-bromo-1-(3-methoxy-3-oxopropyl)-1H-pyrrole-2-carboxylate (4.6 g, 15.9 mmol) in tetrahydrofuran (100 mL) cooled to 0° C. was added a solution of lithium hydroxide (950 mg, 39.6 mmol) in water (50 mL). The reaction was stirred at room temperature for 4 hours. The reaction mixture was acidified to pH=3 by dropwise addition of concentrated hydrochloric acid. The products were extracted with ethyl acetate. The combined organic layers were washed with brine, dried over anhydrous sodium sulfate and concentrated to give 4-bromo-1-(2-carboxyethyl)-1H-pyrrole-2-carboxylic acid (2.9 g, 11.1 mmol, 70% yield) as a colourless solid. The intermediate was used without purification in the next step. 1H NMR (400 MHz, DMSO-d6): δ 12.49 (bs, 2H), 7.25 (s, 1H), 6.82 (s, 1H), 4.45 (t, 2H), 2.70 (t, 2H). LCMS (Method 5): Retention Time=1.95 minutes, [M−2H]−=260.

### Synthesis of 6-bromo-2,3-dihydro-1H-pyrrolizin-1-one

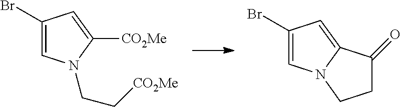 [SMILES: COC(=O)CCn1cc(Br)cc1C(=O)OC ; O=C1CCn2cc(Br)cc21]

To a solution of 4-bromo-1-(2-carboxyethyl)-1H-pyrrole-2-carboxylic acid (0.7 g, 2.7 mmol) in acetic anhydride (20 mL) was added sodium acetate (154 mg, 1.9 mmol) and the reaction mixture was heated to 100° C. for 15 hours. The cooled reaction mixture was cooled and treated with ice cold water and the products were extracted with ethyl acetate. The combined organic layers were washed with brine, dried over anhydrous sodium sulfate and concentrated under reduced pressure. The crude material was purified by column chromatography on silica eluting with 20-40% ethyl acetate in petroleum ether gradient to give 6-bromo-2,3-dihydro-1H-pyrrolizin-1-one (290 mg, 1.5 mmol, 63% yield) as a pale-yellow solid. 1H NMR (400 MHz, DMSO-d6): δ 7.51 (s, 1H), 6.75 (s, 1H), 4.31 (t, 2H), 2.97 (t, 2H). LCMS (Method 5): Retention Time=1.87 minutes, [MH]+=201.

### Synthesis of 6-(4,4,5,5-Tetramethyl-1,3,2-dioxaborolan-2-yl)-2,3-dihydro-1H-pyrrolizin-1-one

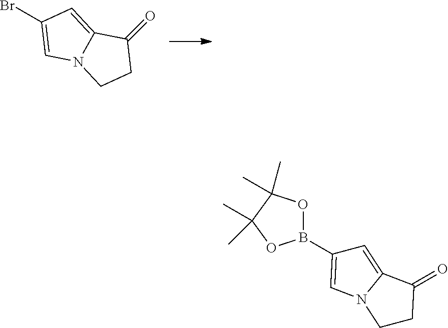 [SMILES: O=C1CCn2cc(Br)cc21 ; CC1(C)OB(c2cc3n(c2)CCC3=O)OC1(C)C]

To a degassed solution of 6-bromo-2,3-dihydropyrrolizin-1-one (600 mg, 3.0 mmol) in N,N-dimethylformamide (10 mL) was added potassium acetate (736 mg, 7.5 mmol), [1,1′-bis(diphenylphosphino)ferrocene]dichloropalladium(II), complex with dichloromethane (244 mg, 0.3 mmol) and bis(pinacolato)diboron (1.1 g, 4.5 mmol). The microwave vial was sealed and evacuated under vacuum and backfilled with nitrogen gas, this was repeated twice more, and the reaction mixture was heated to 100° C. for 2 hours under microwave irradiation. The reaction mixture was partitioned between ethyl acetate and water. The organic layer was washed with brine, dried over anhydrous sodium sulfate and concentrated. The crude material was purified by column chromatography on silica gel eluting with 30-50% ethyl acetate in petroleum ether gradient to give 6-(4,4,5,5-tetramethyl-1,3,2-dioxaborolan-2-yl)-2,3-dihydro-1H-pyrrolizin-1-one (145 mg, 0.6 mmol, 19%) as a solid. 1H NMR (400 MHz, CDCl3): δ 7.40 (s, 1H), 7.05 (s, 1H), 4.32 (t, 2H), 3.13 (t, 2H), 1.34 (s, 12H). LCMS (Method 5): Retention Time=2.22 minutes, [MH]+=248.

### Synthesis of benzyl N-(but-3-en-1-yl)carbamate

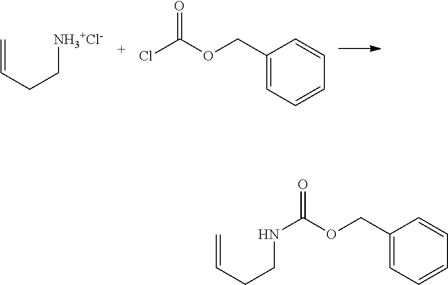

To a solution but-3-en-1-amine hydrochloride (10 g, 93.0 mmol) in dichloromethane (1 L) cooled to 0° C. was added sodium carbonate (4 M aqueous solution, 90 mL, 360.0 mmol) and the reaction was stirred at 0° C. for 15 minutes before dropwise addition of benzyl chloroformate (15.9 g, 93.0 mmol). The reaction mixture was stirred at room temperature for 16 hours. After this time, the reaction mixture was poured into water the layers were separated and the products were extracted with ethyl acetate. The combined organic layers were dried over anhydrous sodium sulfate and concentrated. The crude material was purified by column chromatography on silica gel eluting with 20-30% ethyl acetate in n-hexane gradient to give benzyl N-(but-3-en-1-yl)carbamate (6.0 g, 29.2 mmol, 31% yield) as a yellow oil. 1H NMR (400 MHz, Chloroform-d) δ 7.45-7.29 (m, 5H), 5.83-5.65 (m, 1H), 5.20-4.96 (m, 4H), 4.78 (s, 1H), 3.35-3.17 (m, 2H), 2.37-2.17 (m, 2H). LCMS (Method 4-Column 1): Retention Time=1.94 minutes, [MH]+=206.

### Synthesis of benzyl (1R,5S,7S)-7-(hydroxymethyl)-6-oxa-2-azabicyclo[3.2.1]octane-2-carboxylate (Isomer 1) and benzyl (1S,5R,7S)-7-(hydroxymethyl)-6-oxa-2-azabicyclo[3.2.1]octane-2-carboxylate (Isomer 2)

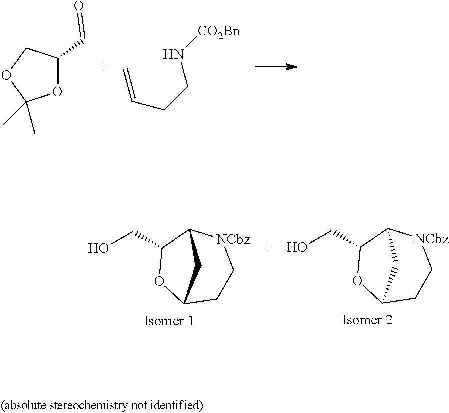

To a solution of benzyl N-(but-3-en-1-yl)carbamate (3.0 g, 14.6 mmol) and 2,3-isopropylidene-(R)-glyceraldeyde (2.3 g, 17.5 mmol) in acetonitrile (250 mL) was added p-toluenesulfonic acid monohydrate (3.9 g, 20.5 mmol). The reaction was stirred at room temperature for 16 hours. After this time additional 2,3-isopropylidene-(R)-glyceraldeyde (2.3 g, 17.5 mmol) and p-toluenesulfonic acid monohydrate (3.9 g, 20.5 mmol) were added. After a further 16 hours, the volatiles were removed under reduced pressure and the crude material was purified by preparative HPLC eluting with isopropanol and heptane to give Isomer 1: benzyl (1R,5S,7S)-7-(hydroxymethyl)-6-oxa-2-azabicyclo[3.2.1]octane-2-carboxylate (280 mg, 1.0 mmol, 7% yield) and Isomer 2: benzyl (1S,5R,7S)-7-(hydroxymethyl)-6-oxa-2-azabicyclo[3.2.1]octane-2-carboxylate (560 mg, 2.0 mmol, 14% yield) as clear oils. Relative stereochemistry arbitrarily assigned.

### Isomer 1: (benzyl (1R,5S,7S)-7-(hydroxymethyl)-6-oxa-2-azabicyclo[3.2.1]octane-2-carboxylate)

1H NMR (400 MHz, Chloroform-d) δ 7.43-7.29 (m, 5H), 5.13 (s, 2H), 4.76-4.52 (m, 2H), 4.16 (dd, 1H), 4.10-3.90 (m, 1H), 3.69-3.53 (m, 1H), 3.53-3.42 (m, 1H), 3.39-3.22 (m, 1H), 2.08-1.85 (m, 1H), 1.84-1.66 (m, 3H). LCMS (Method 4-Column 1): Retention Time=1.41 minutes, [MH]+=278.

### Isomer 2: benzyl (1S,5R,75)-7-(hydroxymethyl)-6-oxa-2-azabicyclo[3.2.1]octane-2-carboxylate

1H NMR (400 MHz, Chloroform-d) δ 7.40-7.30 (m, 5H), 5.18 (s, 2H), 4.69 (br s, 1H), 4.59-4.46 (m, 1H), 4.00 (ddd, 1H), 3.71 (dd, 1H), 3.50-3.34 (m, 2H), 2.17-2.00 (m, 1H), 1.92-1.68 (m, 3H). LCMS (Method 4-Column 1): Retention Time=1.40 minutes, [MH]+=278.

### Synthesis of 1-[(benzylamino)methyl]cyclobutan1-ol

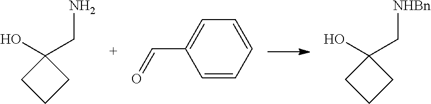

To a solution of benzaldehyde (4.5 g, 42.4 mmol) in ethanol (150 mL) was added 1-(aminomethyl)cyclobutanol (4.3 g, 42.4 mmol) and the reaction mixture was stirred at room temperature for 2 hours. After this time the reaction mixture was cooled to 0° C. and sodium borohydride (1.8 g, 46.6 mmol) was added portion-wise. The reaction mixture was warmed to room temperature and stirring was continued for 16 hours. The reaction mixture was quenched by addition of ice water and the products were extracted with ethyl acetate. The combined organic layers were washed with brine, dried over anhydrous sodium sulphate and concentrated to give 1-[(benzylamino)methyl]cyclobutan-1-ol (52 g, 27.2 mmol, 64% yield) as a colourless solid. The intermediate was used in the next step without further purification. 1H NMR (300 MHz, DMSO-d6) S 7.42-7.02 (m, 5H), 4.89 (s, 1H), 3.74 (s, 2H), 2.54-2.51 (m, 1H), 2.04-1.71 (m, 4H), 1.71-1.49 (m, 1H), 1.49-1.26 (m, 1H). LCMS (Method 5): Retention Time=1.59 minutes, [MH+]=192.

### Synthesis of [(6S)-8-benzyl-5-oxa-8-azaspiro[3.5]nonan-6-yl]methanol

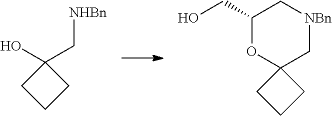

To a solution of 1-[(benzylamino)methyl]cyclobutan1-ol (9.5 g, 49.7 mmol) in toluene (200 mL) was added (R)-(−)-epichlorohydrin (6.9 g, 74.5 mmol) and lithium perchlorate (5.3 g, 49.7 mmol) and the reaction mixture was stirred at room temperature for 3 days. Sodium methoxide (25% w/w methanolic solution, 16 mL, 74.5 mmol) was added and the reaction was stirred at room temperature for a further 1 day. The reaction mixture was quenched by addition of ice-cold water and the products were extracted with ethyl acetate. The combined organic layers were washed with brine, dried over anhydrous sodium sulfate and concentrated. The crude material was purified by column chromatography on silica gel eluting with 20-60% ethyl acetate in petroleum ether gradient to give [(6S)-8-benzyl-5-oxa-8-azaspiro[3.5]nonan-6-yl]methanol (10.0 g, 40.4 mmol, 81% yield) as an oil. LCMS (Method 5): Retention Time=1.64 minutes, [MH]+=248.

### Synthesis of [(6S)-5-oxa-8-azaspiro[3.5]nonan-6-yl]methanol

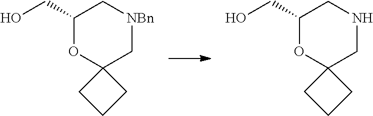 [SMILES: OC[C@@H]1CN(Cc2ccccc2)CC2(CCC2)O1 ; OC[C@@H]1CNCC2(CCC2)O1]

To a solution of [(6S)-8-benzyl-5-oxa-8-azaspiro[3.5]nonan-6-yl]methanol (10.0 g, 40.4 mmol) in methanol (200 mL) under nitrogen was added palladium hydroxide on carbon (20% loading wet support, 2.0 g, 1.4 mmol). The reaction flask was evacuated and refilled with hydrogen gas. After 6 hours, the reaction mixture was filtered through Celite®, washing with additional methanol. The combined filtrates were concentrated under reduced pressure to give [(6S)-5-oxa-8-azaspiro[3.5]nonan-6-yl]methanol (5.6 g, 35.6 mmol, 88% yield) as a yellow oil. The intermediate was used without purification in the next step. LCMS (Method 5): Retention Time=0.80 minutes, [MH]+=158.

### Synthesis of tert-butyl (6S)-6-(hydroxymethyl)-5-oxa-8-azaspiro[3.5]nonane-8-carboxylate

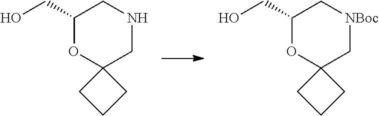 [SMILES: OC[C@@H]1CNCC2(CCC2)O1 ; CC(C)(C)OC(=O)N1C[C@@H](CO)OC2(CCC2)C1]

To a solution of [(6S)-5-oxa-8-azaspiro[3.5]nonan-6-yl]methanol (5.6 g, 35.6 mmol) in dichloromethane (100 mL) and water (50 mL) was added a sodium hydroxide solution (2 M aqueous, 18 mL, 36.0 mmol) and di-tert-butyl decarbonate (7.8 g, 35.6 mmol) and the reaction mixture was stirred at room temperature for 5 hours. The reaction mixture was partitioned between water and dichloromethane. The combined organic layers were washed with brine, dried over anhydrous sodium sulfate and concentrated. The crude material was purified by column chromatography on silica gel, eluting with 10-20% ethyl acetate in n-heptane gradient to give tert-butyl (6S)-6-(hydroxymethyl)-5-oxa-8-azaspiro[3.5]nonane-8-carboxylate (4.5 g, 17.5 mmol, 49% yield) as an oil. 1H NMR (400 MHz, DMSO-d6) δ 4.73 (t, 1H), 4.04-3.72 (m, 2H), 3.46-3.36 (m, 1H), 3.35-3.21 (m, 2H), 2.01-1.70 (m, 5H), 1.67-1.49 (m, 1H), 1.41 (s, 9H). LCMS (Method 5): Retention Time=2.01 minutes, [(M−100)H]+=158.

### Synthesis of 3-{benzyl[(2S)-3-(benzyloxy)-2-hydroxypropyl]amino}propan-1-ol

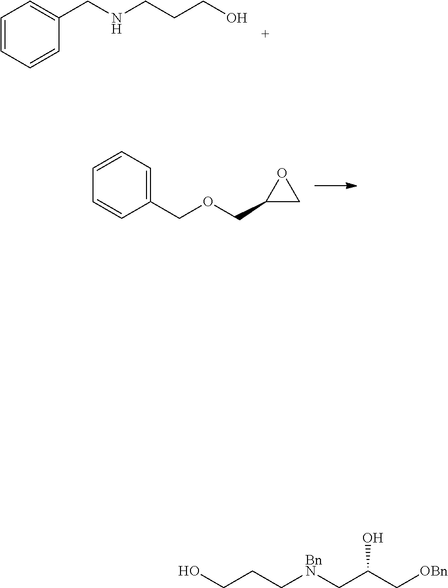 [SMILES: OCCCNCc1ccccc1 ; c1ccc(COC[C@@H]2CO2)cc1 ; OCCCN(Cc1ccccc1)C[C@H](O)COCc1ccccc1]

To a solution of 3-(benzylamino)propan-1-ol (20.0 g, 121.0 mmol) in 2-propanol (200 mL) was added (2S)-2-(phenylmethoxymethyl)oxirane (21.8 g, 132.8 mmol). The reaction was stirred at 40° C. for 16 hours. The reaction mixture was concentrated under reduced pressure and co-distilled with toluene (500 mL). The crude material was purified by column chromatography on neutral alumina eluting with 1% methanol in dichloromethane to give 3-{benzyl[(2S)-3-(benzyloxy)-2-hydroxypropyl]amino}propan-1-ol (31.0 g, 94.1 mmol, 78% yield) as a gummy liquid. 1H NMR (400 MHz, Chloroform-d) δ 7.45-7.17 (m, 10H), 4.53 (s, 2H), 4.06-3.95 (m, 1H), 3.81-3.67 (m, 3H), 3.56 (d, 1H), 3.48 (dd, 1H), 3.41 (dd, 1H), 2.78 (ddd, 1H), 2.72-2.59 (m, 2H), 2.53 (dd, 1H), 1.87-1.64 (m, 2H). LCMS (Method 4-Column 2): Retention Time=1.34 minutes, [MH]+=330.

### Synthesis of 3-{benzyl[(2S)-3-(benzyloxy)-2-hydroxypropyl]amino}propyl methanesulfonate

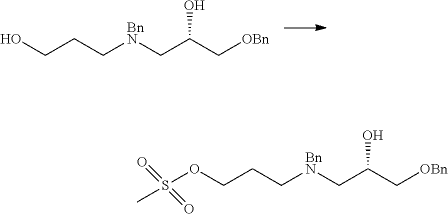 [SMILES: OCCCN(Cc1ccccc1)C[C@H](O)COCc1ccccc1 ; CS(=O)(=O)OCCCN(Cc1ccccc1)C[C@H](O)COCc1ccccc1]

To a solution of 3-{benzyl[(2S)-3-(benzyloxy)-2-hydroxypropyl]amino}propan-1-ol (30.0 g, 91.1 mmol) in dichloromethane (300 mL) cooled to −6° C., was added N,N-diisopropylethylamine (16.0 mL, 91.1 mmol) and then after 5 minutes methanesulfonyl chloride (7.1 mL, 91.1 mmol) was added. The reaction mixture was stirred at −6° C. for further 30 minutes. The reaction mixture was poured onto ice and then diluted with aqueous saturated sodium bicarbonate solution and the products were extracted with dichloromethane. The combined organic layers were dried over anhydrous sodium sulfate and concentrated to give 3-{benzyl[(2S)-3-(benzyloxy)-2-hydroxypropyl]amino}propyl methanesulfonate (37.0 g, 90.8 mmol, 100% yield) as a gummy liquid. 1H NMR (400 MHz, Chloroform-d) δ 7.41-7.23 (m, 10H), 4.56 (s, 2H), 4.29-4.20 (m, 2H), 3.98-3.87 (m, 1H), 3.76 (d, 1H), 3.58-3.39 (m, 3H), 3.14 (s, 1H), 2.93 (s, 3H), 2.77-2.65 (m, 1H), 264-2.51 (m, 3H), 1.97-1.84 (m, 2H). LCMS (Method 4-Column 7): Retention Time=1.46 minutes, [MH]+=408.

### Synthesis of (2S)-4-benzyl-2-[(benzyloxy)methyl]-1,4-oxazepane

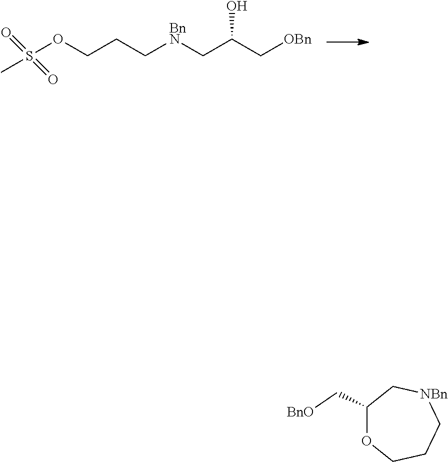 [SMILES: CS(=O)(=O)OCCCN(Cc1ccccc1)C[C@H](O)COCc1ccccc1 ; c1ccc(COC[C@@H]2CN(Cc3ccccc3)CCCO2)cc1]

To a suspension of sodium hydride (57-63% w/w oil dispersion, 3.1 g, 127.1 mmol) in tetrahydrofuran (400 mL) cooled to 0° C. was added a solution of 3-{benzyl[(2S)-3-(benzyloxy)-2-hydroxypropyl]amino}propyl methanesulfonate (37.0 g, 90.8 mmol) in tetrahydrofuran (100 mL). The reaction was stirred at room temperature for 16 hours. The reaction mixture was cooled to 0° C. and quenched by addition of a saturated aqueous solution of sodium bicarbonate and the products were extracted with ethyl acetate. The combined organic layers were dried over anhydrous sodium sulfate and concentrated. The crude material was purified column chromatography on silica gel, eluting with 20% ethyl acetate in hexane to give (2S)-4-benzyl-2-[(benzyloxy)methyl]-1,4-oxazepane (12.3 g, 39.5 mmol, 43% yield). 1H NMR (400 MHz, DMSO-d6) δ 7.54-6.92 (m, 10H), 4.49-4.24 (m, 2H), 3.86-3.64 (m, 3H), 3.61 (s, 2H), 3.42-3.35 (m, 1H), 3.31-3.15 (m, 1H), 2.84 (d, 1H), 2.79-2.64 (m, 1H), 2.47-2.41 (m, 1H), 2.35 (dd, 1H), 1.92-1.76 (m, 1H), 1.76-1.60 (m, 1H). LCMS: Retention Time=7.26 minutes, [MH]+=312. The sample was analysed using the following method: Waters Alliance 2690 and 996 PDA detector with Micro mass ZQ LCMS. Column: Sunfire C18, 150×4.6 mm, 3.5 micron. Column temperature: 35° C. Mobile Phase A: 5 mM ammonium acetate with 0.1% formic acid in Milli-Q water. Mobile Phase B: methanol. Mobile phase gradient details: T=0 minutes (90% A, 10% B; gradient to T=7 minutes (40% A, 60% B); gradient to T=9 minutes (0% A, 100% B); T=14 minutes (0% A, 100% B); gradient to T=14.01 minutes (90% A, 10% B); end of run at T=17 minutes (90% A, 10% B). Flow rate: 1 mL/min, analysis time 17 minutes.

### Synthesis of tert-butyl (2S)-2-(hydroxymethyl)-1,4-oxazepane-4-carboxylate

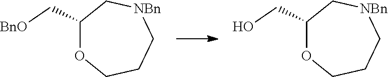 [SMILES: c1ccc(COC[C@@H]2CN(Cc3ccccc3)CCCO2)cc1 ; OC[C@@H]1CN(Cc2ccccc2)CCCO1]

To solution of (2S)-4-benzyl-2-[(benzyloxy)methyl]-1,4-oxazepane (11.3 g, 36.3 mmol) in ethanol (120 mL) were added di-tert-butyl dicarbonate (10.0 mL, 43.5 mmol) and palladium on carbon (10% w/w, 17.0 g, 16.0 mmol). The reaction mixture was placed under an atmosphere of hydrogen and stirred at room temperature for 16 hours. The reaction mixture was filtered through bed of Celite® washing with ethanol (500 mL). The filtrate was concentrated under reduced pressure. The crude material was purified by column chromatography on silica gel eluting with 2% methanol in dichloromethane to give tert-butyl (2S)-2-(hydroxymethyl)-1,4-oxazepane-4-carboxylate (6.7 g, 29.0 mmol, 80% yield) as a gummy liquid. 1H NMR (400 MHz, DMSO-d6) δ 4.78-4.64 (m, 1H), 4.01-3.86 (m, 1H), 3.78-3.62 (m, 1H), 3.62-3.46 (m, 1H), 3.46-3.16 (m, 4H), 3.07-2.86 (m, 1H), 1.84-1.64 (m, 2H), 1.39 (s, 9H). LCMS (Method 4-Column 9): Retention Time=1.48 minutes, [(M−100)H]+=132. HPLC: Retention Time=5.21 minutes.

### Synthesis of 3-{benzyl[(2R)-3-(benzyloxy)-2-hydroxypropyl]amino}propan-1-ol

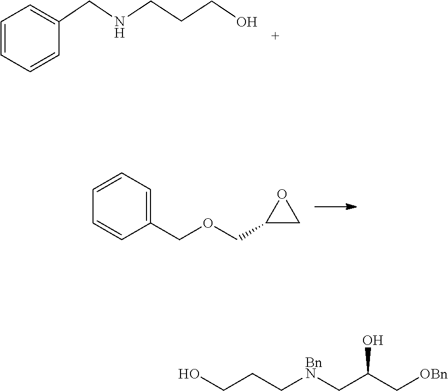 [SMILES: OCCCNCc1ccccc1 ; c1ccc(COC[C@H]2CO2)cc1 ; OCCCN(Cc1ccccc1)C[C@@H](O)COCc1ccccc1]

To solution of 3-(benzylamino)propan-1-ol (10.0 g, 60.5 mmol) in 2-propanol (100 mL) was added (2R)-2-(phenylmethoxymethyl)oxirane (9.9 g, 60.5 mmol). The reaction mixture was stirred at 40° C. for 16 hours. The reaction mixture was concentrated under reduced pressure. The crude material was purified by column chromatography on neutral alumina to give 3-{benzyl[(2R)-3-(benzyloxy)-2-hydroxypropyl]amino}propan-1-ol (16.0 g, 48.6 mmol, 80% yield) as a colourless liquid. 1H NMR (400 MHz, Chloroform-d) δ 7.45-7.23 (m, 10H), 4.53 (s, 2H), 4.06-3.92 (m, 1H), 3.81-3.67 (m, 3H), 3.57 (d, 1H), 3.48 (dd, 1H), 3.41 (dd, 1H), 2.78 (ddd, 1H), 2.72-2.58 (m, 2H), 2.54 (dd, 1H), 1.87-1.64 (m, 2H). LCMS (Method 4-Column 5): Retention Time=1.36 minutes, [MH]+=330.

### Synthesis of 3-{benzyl[(2R)-3-(benzyloxy)-2-hydroxypropyl]amino}propyl methanesulfonate

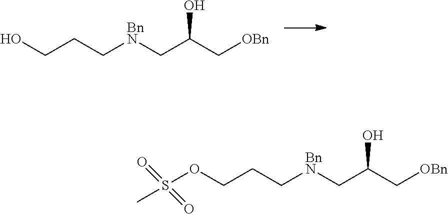 [SMILES: OCCCN(Cc1ccccc1)C[C@@H](O)COCc1ccccc1 ; CS(=O)(=O)OCCCN(Cc1ccccc1)C[C@@H](O)COCc1ccccc1]

To solution of 3-{benzyl[(2R)-3-(benzyloxy)-2-hydroxypropyl]amino}propan-1-ol (16.0 g, 48.6 mmol) in dichloromethane (150 mL) cooled to −6° C. was added N,N-diisopropylethylamine (6.8 mL, 48.6 mmol) and then after 5 minutes was added methanesulfonyl chloride (3.8 mL, 48.6 mmol). The reaction was stirred at −6° C. for a further 30 minutes. The reaction mixture was poured onto ice and then diluted with a saturated aqueous sodium bicarbonate solution. The products were extracted with dichloromethane. The combined organic layers were dried over anhydrous sodium sulfate and concentrated to give 3-{benzyl[(2R)-3-(benzyloxy)-2-hydroxypropyl]amino}propyl methanesulfonate (17.0 g, 25.4 mmol, 52% yield) as a colourless liquid. 1H NMR (400 MHz, Chloroform-d) δ 7.41-7.26 (m, 10H), 4.55 (s, 2H), 4.27-4.20 (m, 2H), 3.99-3.88 (m, 1H), 3.80 (d, 1H), 3.64-3.40 (m, 3H), 2.93 (s, 3H), 2.80-2.66 (m, 1H), 2.69-2.54 (m, 3H), 2.01-1.87 (m, 2H). LCMS (Method 4-Column 7): Retention Time=1.46 minutes, [MH]+=408.

### Synthesis of (2R)-4-benzyl-2-[(benzyloxy)methyl]-1,4-oxazepane

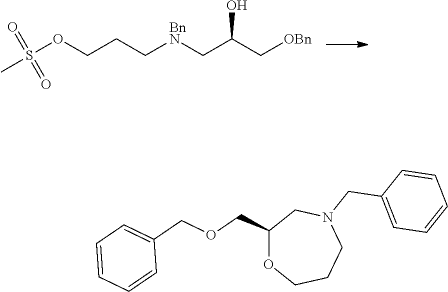 [SMILES: CS(=O)(=O)OCCCN(Cc1ccccc1)C[C@@H](O)COCc1ccccc1 ; c1ccc(COC[C@H]2CN(Cc3ccccc3)CCCO2)cc1]

To a solution of 3-{benzyl[(2R)-3-(benzyloxy)-2-hydroxypropyl]amino}propyl methanesulfonate (17.5 g, 42.9 mmol) in tetrahydrofuran (200 mL) cooled to 0° C. was added portion-wise sodium hydride (2.4 g, 60.1 mmol). The reaction mixture was stirred at room temperature for 16 hours. The reaction mixture was quenched by addition of a saturated aqueous sodium bicarbonate solution and the products were extracted with ethyl acetate. The combined organic layers were dried over anhydrous sodium sulfate and concentrated. The crude material was purified by reverse phase column chromatography eluting with a 50-75% acetonitrile in water gradient. The isolated material was further purified by column chromatography on silica gel eluting with 10-40% ethyl acetate in hexane gradient to give (2R)-4-benzyl-2-[(benzyloxy)methyl]-1,4-oxazepane (3.1 g, 9.4 mmol, 22% yield) as a colourless liquid. 1H NMR (400 MHz, DMSO-d6) δ 7.40-7.17 (m, 10H), 4.47-4.27 (m, 2H), 3.82-3.71 (m, 2H), 3.71-3.63 (m, 1H), 3.61 (s, 2H), 3.40-3.35 (m, 1H), 3.24 (dd, 1H), 2.85 (d, 1H), 2.79-2.66 (m, 1H), 2.49-2.44 (m, 1H), 2.36 (dd, 1H), 1.89-1.75 (m, 1H), 1.78-1.64 (m, 1H). LCMS (Method 8-Column 2): Retention Time=7.23 minutes, [MH]+=312.

### Synthesis of tert-butyl (2R)-2-(hydroxymethyl)-1,4-oxazepane-4-carboxylate

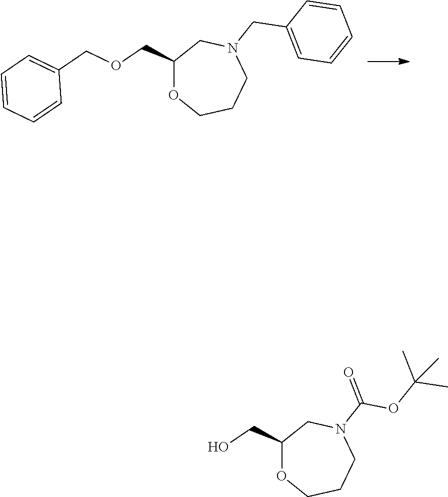 [SMILES: c1ccc(COC[C@H]2CN(Cc3ccccc3)CCCO2)cc1 ; CC(C)(C)OC(=O)N1CCCO[C@@H](CO)C1]

To a solution of (2R)-4-benzyl-2-[(benzyloxy)methyl]-1,4-oxazepane (2.8 g, 9.0 mmol) in ethanol (30 mL) were added di-tert-butyl dicarbonate (2.4 g, 10.8 mmol) and palladium on carbon (10% w/w, 2.8 g, 2.6 mmol). The reaction mixture was stirred under 200 psi of hydrogen at room temperature for 14 hours. The reaction mixture was filtered through Celite® washing with ethyl acetate. The resulting filtrate was concentrated under reduced pressure. The crude material was purified by column chromatography on silica gel eluting with 2-4% methanol in dichloromethane gradient to give tert-butyl (2R)-2-(hydroxymethyl)-1,4-oxazepane-4-carboxylate (0.97 g, 3.9 mmol, 44% yield) as a colourless liquid. 1H NMR (400 MHz, DMSO-d6) δ 4.83-4.60 (m, 1H), 4.02-3.86 (m, 1H), 3.70 (ddd, 1H), 3.58-3.17 (m, 4H), 2.97 (ddd, 1H), 1.84-1.61 (m, 2H), 1.39 (s, 9H). LCMS (Method 4-Column 9): Retention Time=1.48 minutes, [MNa]+=254. HPLC: Retention Time=5.18 minutes.

### Synthesis of tert-butyl (2S)-2-[methoxy(methyl)carbamoyl]morpholine-4-carboxylate

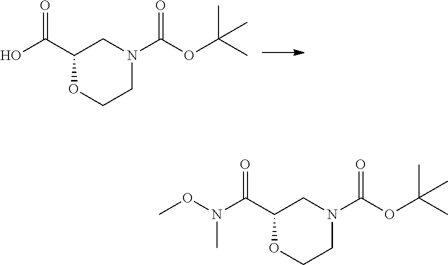 [SMILES: CC(C)(C)OC(=O)N1CCO[C@H](C(=O)O)C1 ; CON(C)C(=O)[C@@H]1CN(C(=O)OC(C)(C)C)CCO1]

To a solution of (S)-4-(tert-butoxycarbonyl)morpholine-2-carboxylic acid (60.0 g, 259.5 mmol) in dichloromethane (1.0 L) were added N,N-diisopropylethyl amine (110 mL, 648.6 mmol) and 1-[bis(dimethylamino)methylene]-1H-1,2,3-triazolo[4,5-b]pyridinium 3-oxide hexafluorophosphate (HATU) (197.2 g, 518.9 mmol) at room temperature. The reaction mixture was stirred at room temperature for 15 minutes then was added portion wise N,O-dimethylhydroxylamine hydrochloride (38.0 g, 389.2 mmol) and the reaction was stirred at room temperature for 16 hours. The reaction mixture was poured into a saturated aqueous sodium bicarbonate solution, the products were extracted with dichloromethane. The combined organic layers were dried over anhydrous sodium sulfate and concentrated to give tert-butyl (2S)-2-[methoxy(methyl)carbamoyl]morpholine-4-carboxylate (165.0 g, 246.6 mmol, 95% yield) as a pale yellow oil. No purification was carried out the material was used crude in the next step LCMS (Method 4-Column 7): Retention Time=1.56 minutes. [MNa]+=297.

### Synthesis of tert-butyl (2S)-2-acetylmorpholine-4-carboxylate

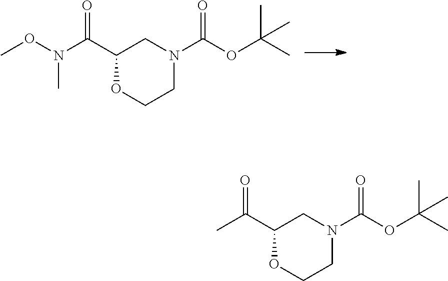

To a solution of tert-butyl (2S)-2-[methoxy(methyl)carbamoyl]morpholine-4-carboxylate (165.0 g, 601.5 mmol) in tetrahydrofuran (800 mL) cooled to 0° C. under a nitrogen atmosphere was added methylmagnesium bromide (3 M in diethyl ether, 275 mL, 825.0 mmol) drop wise. The reaction mixture was stirred at room temperature for 16 hours. The reaction mixture was cooled to 0° C. and quenched by addition of a saturated aqueous ammonium chloride solution. The products were extracted with ethyl acetate, the combined organic layers were dried over anhydrous sodium sulfate and concentrated. The crude material was purified by column chromatography on silica gel eluting with 20% ethyl acetate in n-hexane to give tert-butyl (2S)-2-acetylmorpholine-4-carboxylate (50.0 g, 218.1 mmol, 36% yield) as a pale yellow oil. 1H NMR (400 MHz, Chloroform-d) δ 4.23-4.05 (m, 1H), 3.97 (ddd, 1H), 3.86 (d, 2H), 3.56 (td, 1H), 3.05-2.88 (m, 1H), 2.88-2.65 (m, 1H), 2.23 (s, 3H), 1.46 (s, 9H). LCMS (Method 4-Column 2): Retention Time=2.21 minutes, [MH]+=230.

### Synthesis of tert-butyl (2S)-2-(1-hydroxyethyl)morpholine-4-carboxylate

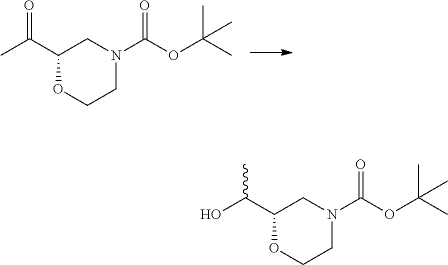 [SMILES: CC(=O)[C@@H]1CN(C(=O)OC(C)(C)C)CCO1 ; CC(O)[C@@H]1CN(C(=O)OC(C)(C)C)CCO1]

To a solution of tert-butyl (2S)-2-acetylmorpholine-4-carboxylate (52.5 g, 229.0 mmol) in methanol (500 mL) cooled to 0° C., was added sodium borohydride (13.1 g, 343.5 mmol) portion wise. The reaction mixture was allowed to warm to room temperature and stirred for 2 hours. The reaction mixture was poured into a saturated aqueous ammonium chloride solution and the volatiles were removed under reduced pressure. The products were extracted with dichloromethane and the combined organic layers were dried over anhydrous sodium sulfate and concentrated to give tert-butyl (2S)-2-(1-hydroxyethyl)morpholine-4-carboxylate as a 1:1 mixture of diastereoisomers (51.0 g, 220.5 mmol, 96% yield) as a pale yellow oil. No purification was carried out, the material was used crude in the next step.

### Isomer 1

LCMS (Method 8-Column 3): Retention Time=10.72 minutes, [MH]+=232.

### Isomer 2

LCMS (Method 8-Column 3): Retention Time=11.41 minutes, [MH]+=232.

### Synthesis of tert-butyl (2S)-2-[(1S)-1-{[(2R)-2-methoxy-2-phenylacetyl]oxy}ethyl]morpholine-4-carboxylate and tert-butyl (2S)-2-[(1R)-1-{[(2R)-2-methoxy-2-phenylacetyl]oxy}ethyl]morpholine-4-carboxylate

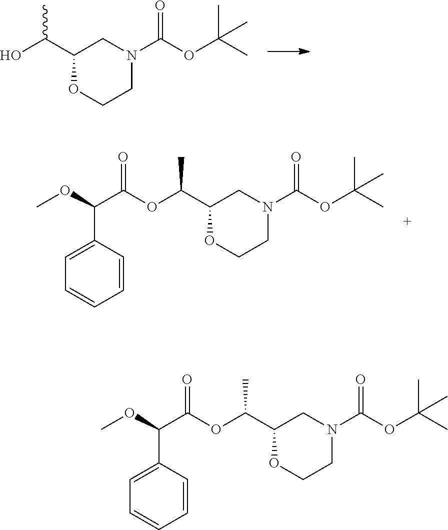

To a solution of tert-butyl (2S)-2-(1-hydroxyethyl)morpholine-4-carboxylate (51.0 g, 220.5 mmol) in dichloromethane (800 mL) were added (2R)-2-methoxy-2-phenylacetic acid (40.3 g, 242.6 mmol) and N,N′-dimethylamino pyridine (6.7 g, 55.1 mmol). The reaction mixture was cooled to 0° C. and N,N′-dicyclohexylcarbodiimide (54.5 g, 264.6 mmol) was added portion wise. The reaction was stirred at room temperature for 16 hours. The reaction mixture was filtrated to remove the N,N′-dicyclohexyl urea by-product and the filtrates were poured into water. The products were extracted with dichloromethane and the combined organic layers were dried over anhydrous sodium sulfate and concentrated. The crude material was purified by column chromatography on silica gel eluting with 8-20% ethyl acetate in n-hexane gradient to give isomer 1: tert-butyl (2S)-2-[(1R)-1-{[(2R)-2-methoxy-2-phenylacetyl]oxy}ethyl]morpholine-4-carboxylate (16.0 g, 42.2 mmols, 19% yield) and isomer 2: tert-butyl (2S)-2-[(1S)-1-{[(2R)-2-methoxy-2-phenylacetyl]oxy}ethyl]morpholine-4-carboxylate (10.3 g, 27.1 mmols, 12% yield).

### Isomer 1: tert-butyl (2S)-2-[(R)-1-{[(?2R)-2-methoxy-2-phenylacetyl]oxy}ethyl]morpholine-4-carboxylate

1H NMR (400 MHz, DMSO-d6) δ 7.40-7.32 (m, 5H), 4.91 (s, 1H), 4.88-4.73 (m, 1H), 4.29 (t, 1H), 3.87-3.58 (m, 3H), 3.33 (s, 3H), 3.29-3.23 (m, 3H), 1.38 (s, 9H), 1.17 (d, 3H). LCMS (Method 4-Column 2): Retention Time=2.88 minutes, [MH]+=380.

### Isomer 2: tert-butyl(2S)-2-[(S)-1-{1[(2R)-2-methoxy-2-phenylacetyl]oxy}ethyl]morpholine-4-carboxylate

1H NMR (400 MHz, DMSO-d6) δ 7.42-7.28 (m, 5H), 5.00-4.91 (m, 1H), 4.90 (s, 1H), 3.81 (d, 1H), 3.76-3.54 (m, 2H), 3.34-3.20 (m, 5H), 2.94-2.73 (m, 1H), 2.73-2.55 (m, 1H), 1.39 (s, 9H), 1.02 (d, 3H). LCMS (Method 4-Column 2): Retention Time=2.92 minutes, [MH]+=380.

### Synthesis of tert-butyl (2S)-2-[(1S)-1-hydroxyethyl]morpholine-4-carboxylate

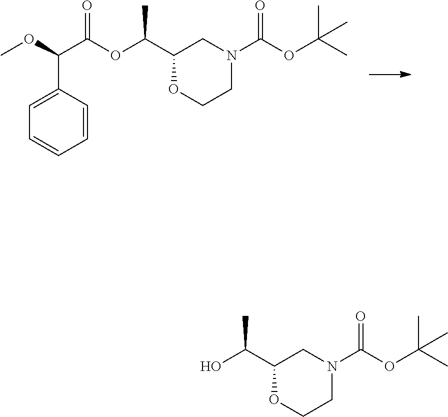 [SMILES: CO[C@@H](C(=O)O[C@@H](C)[C@@H]1CN(C(=O)OC(C)(C)C)CCO1)c1ccccc1 ; C[C@H](O)[C@@H]1CN(C(=O)OC(C)(C)C)CCO1]

To a solution of tert-butyl (2S)-2-[(1S)-1-{[(2R)-2-methoxy-2-phenylacetyl]oxy}ethyl]morpholine-4-carboxylate (9.2 g, 24.3 mmol) in methanol (100 mL) was added potassium carbonate (5.0 g, 36.4 mmol). The reaction was stirred at room temperature for 2 hours. The volatiles were removed under reduced pressure and the reaction was poured into water and the pH of the solution was adjusted to pH=7 using aqueous 1 N hydrochloric acid solution. The products were extracted with dichloromethane and the combined organic layers were dried over anhydrous sodium sulfate and concentrated. The resulting crude material was purified by column chromatography on silica gel eluting with 36% methyl tert-butyl ether in n-heptane to give tert-butyl (2S)-2-[(1S)-1-hydroxyethyl]morpholine-4-carboxylate (4.2 g, 15.4 mmol, 64% yield) as a pale yellow oil. 1H NMR (400 MHz, DMSO-d6) δ 4.68 (d, 1H), 3.86-3.49 (m, 4H), 3.38-3.27 (m, 1H), 3.12 (ddd, 1H), 2.89-2.63 (m, 2H), 1.40 (s, 9H), 1.02 (d, 3H). LCMS (Method 8-column 3): Retention Time=10.82 min, [(M-56)H]+=176.

### Synthesis of tert-butyl (2S)-2-[(1R)-1-hydroxyethyl]morpholine-4-carboxylate

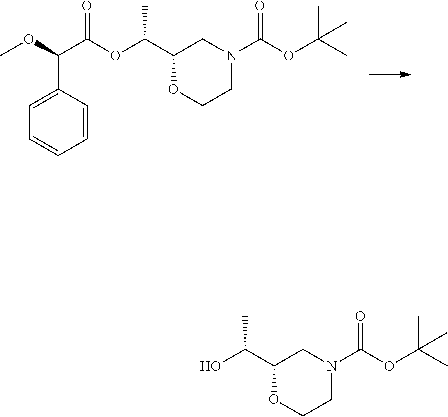 [SMILES: CO[C@@H](C(=O)O[C@H](C)[C@@H]1CN(C(=O)OC(C)(C)C)CCO1)c1ccccc1 ; C[C@@H](O)[C@@H]1CN(C(=O)OC(C)(C)C)CCO1]

To a solution of tert-butyl (2S)-2-[(1R)-1-{[(2R)-2-methoxy-2-phenylacetyl]oxy}ethyl]morpholine-4-carboxylate (11.0 g, 29.0 mmol) in methanol (120 mL) was added potassium carbonate (6.0 g, 43.5 mmol). The reaction mixture was stirred at room temperature for 2 hours. The volatiles were removed under reduced pressure and the reaction was poured into water and the pH of the solution was adjusted to pH=7 using aqueous 1 N hydrochloric acid solution. The products were extracted with dichloromethane and the combined organic layers were dried over anhydrous sodium sulfate and concentrated. The crude material was purified by column chromatography on silica gel eluting with 30% methyl tert-butyl ether in n-heptane to give tert-butyl (2S)-2-[(1R)-1-hydroxyethyl]morpholine-4-carboxylate (4.7 g, 19.5 mmol, 67% yield) as a pale yellow oil. 1H NMR (400 MHz, DMSO-d6) δ 4.78 (d, 1H), 4.01 (d, 1H), 3.80 (d, 1H), 3.69 (d, 1H), 3.47-3.39 (m, 1H), 3.39-3.28 (m, 1H), 2.96 (ddd, 1H), 2.90-2.67 (m, 2H), 1.40 (s, 9H), 1.08 (d, 3H). LCMS (Method 8-Column 3): Retention Time=11.48 minutes, [MH]+=232.

### Synthesis of (2S)-4-benzyl-6,6-dimethylmorpholine-2-carbaldehyde

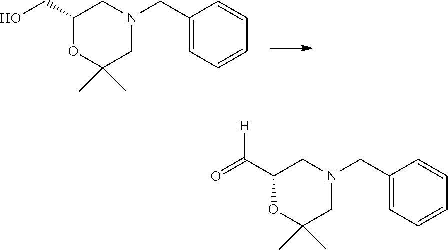 [SMILES: CC1(C)CN(Cc2ccccc2)C[C@@H](CO)O1 ; CC1(C)CN(Cc2ccccc2)C[C@@H](C=O)O1]

To a solution of oxalyl chloride (10.0 mL, 116.9 mmol) in dichloromethane (100 mL) cooled to −78° C. was added dropwise dimethyl sulfoxide (16.6 mL, 233.7 mmol) over 15 minutes. The reaction was stirred at −78° C. for a further 15 minutes. A solution of [(2S)-4-benzyl-6,6-dimethylmorpholin-2-yl]methanol (25.0 g, 106.2 mmols) in dichloromethane (100 mL) was then added over a period of 10 minutes. The reaction mixture was stirred at −78° C. for 30 minutes, then was added triethylamine (74.0 mL, 531.2 mmol) over 10 minutes. Stirring was continued at −78° C. for another 30 minutes. The reaction mixture was quenched with water and the products were extracted with dichloromethane. The combined organic layers were washed with brine, dried over anhydrous sodium sulfate and concentrated to give (2S)-4-benzyl-6,6-dimethylmorpholine-2-carbaldehyde (26.0 g, 111.4 mmol, 105% yield) was taken immediately for the next step without purification. LCMS (Method 5): Retention Time=1.16 minutes, [MH]+=234.

### Synthesis of (1S)-1-[(2S)-4-benzyl-6,6-dimethylmorpholin-2-yl]ethan-1-ol and (1R)-1-[(2S)-4-benzyl-6,6-dimethylmorpholin-2-yl]ethan-1-ol

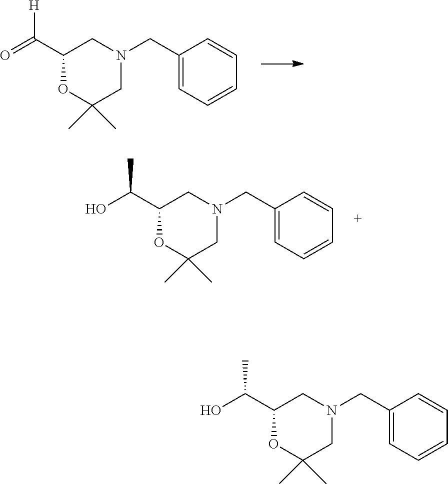

To a solution of (S)-4-benzyl-6,6-dimethylmorpholine-2-carbaldehyde (25.0 g, 107.2 mmol) in tetrahydrofuran (250 mL) cooled to −78° C. under nitrogen was added methyl magnesium bromide (1.4 M in tetrahydrofuran, 115.0 mL, 160.7 mmol). The reaction mixture was stirred at 0° C. for 1-2 hours. The reaction mixture was quenched by addition of a saturated aqueous ammonium chloride solution and the products were extracted with ethyl acetate. The combined organic layers were washed with brine, dried over anhydrous sodium sulfate and concentrated. The crude material was purified by column chromatography in silica gel eluting with 10-16% ethyl acetate in petroleum ether gradient to give (1S)-1-[(2S)-4-benzyl-6,6-dimethylmorpholin-2-yl]ethan-1-ol (6.0 g, 24.1 mmol, 23% yield) as a first eluent as syrupy liquid and (1R)-1-[(2S)-4-benzyl-6,6-dimethylmorpholin-2-yl]ethan-1-ol (5.7 g, 22.9 mmol, 21% yield) as second eluent as syrupy liquid along with 3.5 g of mixed fractions.

### (1S)-1-[(2S)-4-benzyl-6,6-dimethylmorpholin-2-yl]ethan-1-ol

1H NMR (400 MHz, DMSO-d6): δ 7.33-7.31 (m, 4H), 7.27-7.24 (m, 1H), 4.37 (d, 1H), 3.58-3.57 (m, 1H), 3.54-3.51 (m, 1H), 3.49-3.47 (m, 1H), 3.39 (s, 1H), 2.67 (d, 1H), 2.50-2.44 (m, 1H), 1.77-1.68 (m, 2H), 1.25 (s, 3H), 1.06 (s, 3H), 0.97 (d, 3H). LCMS (Method 5): Retention Time=1.27 minutes, [MH]+=250.

### (1R)-1-[(2S)-4-benzyl-6,6-dimethylmorpholin-2-yl]ethan-1-ol

1H NMR (400 MHz, DMSO-d6): δ 7.33-7.31 (m, 4H), 7.27-7.24 (m, 1H), 4.49 (d, 1H), 3.49-3.30 (m, 4H), 2.94 (d, 1H), 2.50-2.46 (m, 1H), 1.73-1.59 (m, 2H), 1.25 (s, 3H), 1.05-1.03 (m, 6H). LCMS (Method 5): Retention Time=1.32 minutes, [MH]+=250.

### Synthesis (1R)-1-[(2S)-6,6-dimethylmorpholin-2-yl]ethan-1-ol

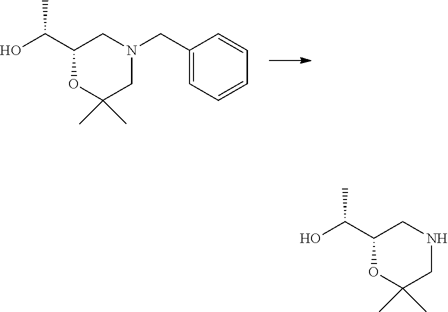 [SMILES: C[C@@H](O)[C@@H]1CN(Cc2ccccc2)CC(C)(C)O1 ; C[C@@H](O)[C@@H]1CNCC(C)(C)O1]

To a solution of (1R)-1-[(2S)-4-benzyl-6,6-dimethylmorpholin-2-yl]ethan-1-ol (5.7 g, 22.9 mmol) in methanol (100 mL) under nitrogen was added palladium hydroxide on carbon (20% loading wet support, 0.9 g, 0.64 mmol) and the reaction was placed under an atmosphere of hydrogen for 2-3 hours. The reaction mixture was filtered through pad of Celite® washing with methanol and the filtrate was concentrated under reduced pressure to give (1R)-1-[(2S)-6,6-dimethylmorpholin-2-yl]ethan-1-ol (3.3 g, 20.7 mmol, 91% yield) The intermediate was taken to the next step without further purification. 1H NMR (400 MHz, MeOD): δ 3.54-3.48 (m, 1H), 3.47-3.43 (m, 1H), 3.05-3.01 (m, 1H), 2.65 (dd, 1H), 2.51 (d, 1H), 2.43-2.37 (m, 1H), 1.29 (s, 3H), 1.17 (s, 3H), 1.15 (d, 3H). LCMS (Method 7): Retention Time=0.22 minutes, [MH]+=160.

### Synthesis of tert-butyl (6S)-6-[(1R)-1-hydroxyethyl]-2,2-dimethylmorpholine-4-carboxylate

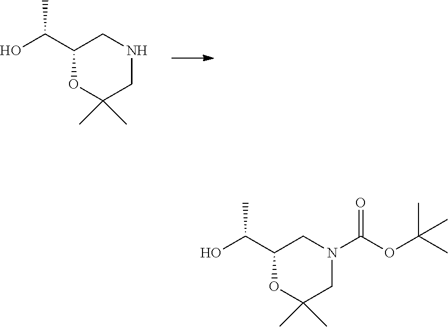 [SMILES: C[C@@H](O)[C@@H]1CNCC(C)(C)O1 ; C[C@@H](O)[C@@H]1CN(C(=O)OC(C)(C)C)CC(C)(C)O1]

To a solution of (1R)-1-[(2S)-6,6-dimethylmorpholin-2-yl]ethan-1-ol (3.3 g, 20.7 mmol) in dichloromethane (100 mL) and water (60 mL) were added sodium hydroxide (2 N, 10.3 mL, 20.7 mmol), followed by a solution of di-tert-butyl dicarbonate (4.5 g, 20.7 mmol) in dichloromethane (30 mL). The reaction was stirred at room temperature for 2 hours. The reaction mixture was partitioned between water and dichloromethane, the aqueous layer was further extracted with dichloromethane and the combined organic layers were washed with brine, dried over anhydrous sodium sulfate, and concentrated. The crude material was purified by column chromatography on silica gel eluting with a ethyl acetate in petroleum ether gradient to give tert-butyl (S)-6-((R)-1-hydroxyethyl)-2,2-dimethylmorpholine-4-carboxylate (4.7 g, 17.9 mmol, 87% yield) as colourless oil. 1H NMR (400 MHz, DMSO-d6) δ 4.67 (d, 1H), 4.16-3.99 (m, 1H), 3.71-3.54 (m, 1H), 3.44-3.28 (m, 1H), 3.21 (ddd, 1H), 2.72-2.27 (m, 2H), 1.40 (s, 9H), 1.10 (s, 3H), 1.08 (s, 3H), 1.05 (d, 3H). LCMS (Method 5): Retention Time=2.68 minutes, [(M-100)H]+=160.

### Synthesis of (1S)-1-[(2S)-6,6-dimethylmorpholin-2-yl]ethan-1-ol

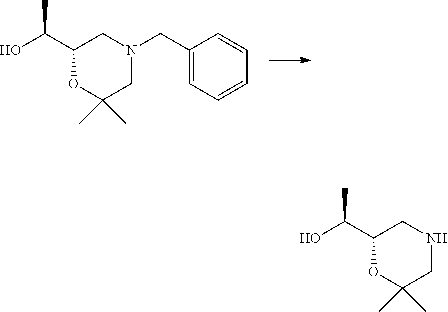 [SMILES: C[C@H](O)[C@@H]1CN(Cc2ccccc2)CC(C)(C)O1 ; C[C@H](O)[C@@H]1CNCC(C)(C)O1]

To a solution of (1S)-1-[(2S)-4-benzyl-6,6-dimethylmorpholin-2-yl]ethan-1-ol (6.0 g, 24.1 mmol) in methanol (100 mL) under nitrogen was added palladium hydroxide on carbon (20% loading wet support, 1.0 g, 0.71 mmol) and the reaction was placed under an atmosphere of hydrogen for 2-3 hours. The reaction mixture was filtered through pad of Celite®, washing with methanol and the filtrate was concentrated under reduced pressure to give (1S)-1-[(2S)-6,6-dimethylmorpholin-2-yl]ethan-1-ol (3.7 g, 23.2 mmol, 97% yield). The intermediate was taken through to the next step without any further purification. 1H NMR (400 MHz, MeOD): δ 3.59-3.50 (m, 2H), 2.83-2.79 (m, 1H), 2.67-2.64 (m, 1H), 2.53-2.50 (m, 1H), 2.50-2.41 (m, 1H), 1.30 (s, 3H), 1.17 (s, 3H), 1.13 (d, 3H). LCMS (Method 5): Retention Time=0.64 minutes, [MH]+=160.

### Synthesis of tert-butyl (6S)-6-[(1S)-1-hydroxyethyl]-2,2-dimethylmorpholine-4-carboxylate

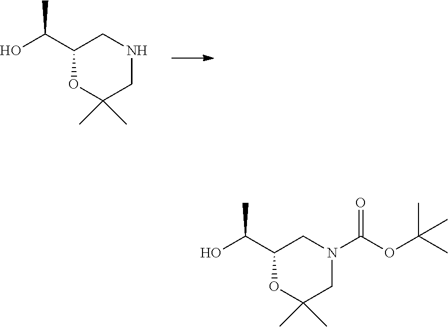 [SMILES: C[C@H](O)[C@@H]1CNCC(C)(C)O1 ; C[C@H](O)[C@@H]1CN(C(=O)OC(C)(C)C)CC(C)(C)O1]

To a solution of (1S)-1-[(2S)-6,6-dimethylmorpholin-2-yl]ethan-1-ol (3.7 g, 23.2 mmol) in dichloromethane (100 mL) and water (70 mL) was added sodium hydroxide (2 N, 11.5 mL, 23.2 mmol), followed by a solution of di-tert-butyl dicarbonate (5.1 g, 23.2 mmol) in dichloromethane (40 mL). The reaction was stirred at room temperature for 2 hours. The reaction mixture was partitioned between water and dichloromethane, the aqueous layer was further extracted with dichloromethane and the combined organic layers were washed with brine, dried over anhydrous sodium sulfate, and concentrated. The crude material was purified by column chromatography on silica gel eluting with ethyl acetate in petroleum ether gradient to give tert-butyl (6S)-6-[(1S)-1-hydroxyethyl]-2,2-dimethylmorpholine-4-carboxylate (4.5 g, 17.4 mmol, 75% yield) as colourless oil. 1H NMR (400 MHz, DMSO-d6) δ 4.51 (d, 1H), 3.88-3.73 (m, 1H), 3.72-3.57 (m, 1H), 3.57-3.48 (m, 1H), 3.43 (ddd, 1H), 2.70-2.52 (m, 2H), 1.40 (s, 10H), 1.12 (s, 3H), 1.09 (s, 3H), 1.00 (d, 3H). LCMS (Method 5): Retention Time=2.65 minutes, [(M−100)H]+=160.

### Synthesis of 4-methyl-2-[2-oxo-2-(propan-2-yloxy)ethyl]pyridine-3-carboxylic acid

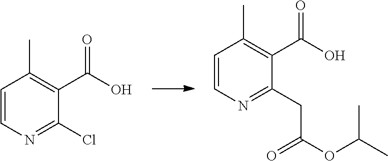 [SMILES: Cc1ccnc(Cl)c1C(=O)O ; Cc1ccnc(CC(=O)OC(C)C)c1C(=O)O]

To a solution of potassium tert-butoxide (6.5 g, 57.7 mmol) in 2-propanol (43 mL) was added ethyl acetoacetate (5.0 g, 38.5 mmol) at room temperature. The reaction mixture was stirred for 1 hour. Copper(II) acetate (350 mg, 1.92 mmol) and 2-chloro-4-methylpyridine-3-carboxylic acid (3.3 g, 19.2 mmol) were added and reaction mixture was heated to 80° C. for 3 hours. The reaction mixture was acidified with acetic acid and the volatiles were removed under reduce pressure. The residue was poured into water and the products were extracted with ethyl acetate. The combined organic layers were dried over anhydrous sodium sulfate and concentrated. The crude material was triturated with mixture of hexane and diethyl ether to give 4-methyl-2-[2-oxo-2-(propan-2-yloxy)ethyl]pyridine-3-carboxylic acid (3.6 g, 13.7 mmol, 71% yield) as a brown solid. 1H NMR (400 MHz, DMSO-d6) δ 13.48 (br s, 1H), 8.37 (s, 1H), 7.25 (s, 1H), 4.88 (hept, 1H), 3.83 (s, 2H), 2.35 (s, 3H), 1.16 (d, 6H). LCMS (Method 4-Column 7): Retention Time=0.90 minutes, [MH]+=238.

### Synthesis of 4-methyl-5,6,7,8-tetrahydro-1,6-naphthyridine-5,7-dione

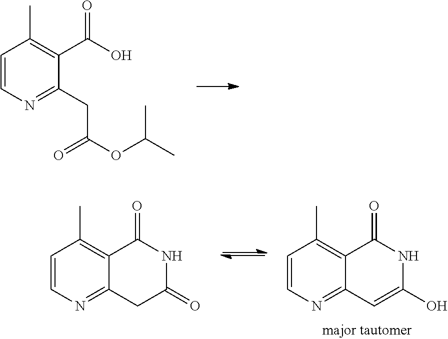 [SMILES: Cc1ccnc(CC(=O)OC(C)C)c1C(=O)O ; Cc1ccnc2c1C(=O)NC(=O)C2 ; Cc1ccnc2cc(O)[nH]c(=O)c12]

To a solution of 4-methyl-2-[2-oxo-2-(propan-2-yloxy)ethyl]pyridine-3-carboxylic acid (1.5 g, 5.7 mmol) in tetrahydrofuran (10 mL) cooled to 0° C., was added triethylamine (1.2 mL, 8.0 mmol) and the reaction was stirred for 20 minutes. Ethyl chloroformate (0.7 mL, 7.4 mmol) was added dropwise maintaining the temperature at 0° C. and the reaction was stirred at room temperature for 48 hours. The reaction mixture was cooled to ° C. and ammonium hydroxide (25-30% in water, 10.0 mL, 5.7 mmol) was added dropwise. The reaction was stirred at room temperature for a further 2 hours. The reaction mixture was neutralised with dilute aqueous hydrochloric acid solution and the volatiles were removed under reduce pressure. The crude material was purified by reverse phase column chromatography eluting with water and acetonitrile as the mobile phase to give 4-methyl-5,6,7,8-tetrahydro-1,6-naphthyridine-5,7-dione (250 mg, 1.18 mmol, 21% yield) as a light brown solid. 1H NMR (400 MHz, DMSO-d6) δ 10.17 (s, 1H), 7.78 (s, 1H), 6.24 (s, 1H), 4.98 (s, 1H), 4.05 (s, 1H), 2.61 (s, 3H). LCMS (Method 4-Column 7): Retention Time=0.73 minutes, [MH]+=177.

### Synthesis of 5,7-dichloro-4-methyl-1,6-naphthyridine

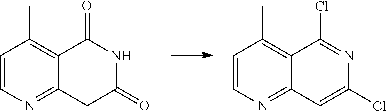

To a mixture of 4-methyl-5,6,7,8-tetrahydro-1,6-naphthyridine-5,7-dione (250 mg, 1.4 mmol) and tetramethylammonium chloride (160 g, 1.5 mmol) was added phosphoryl chloride (2.4 mL, 18.4 mmol). The reaction mixture was heated to 110° C. for 16 hours. The cooled reaction mixture was concentrated under reduce pressure and azeotroped with toluene. The residue was poured into cold water, basified with saturated aqueous sodium bicarbonate solution and the products were extracted with ethyl acetate. The combined organic layer was dried over anhydrous sodium sulfate and concentrated. The crude material was purified by column chromatography on silica gel eluting with an ethyl acetate in hexane gradient to give 5,7-dichloro-4-methyl-1,6-naphthyridine (30 mg, 0.13 mmol, 9% yield) as off colourless solid. 1H NMR (400 MHz, DMSO-d6) S 8.99 (d, 1H), 8.08 (s, 1H), 7.64 (d, 1H), 3.01 (s, 3H). LCMS (Method 4-Column 7): Retention Time=1.78 minutes, [MH]+=213. HPLC: Retention Time=7.98 minutes.

### Synthesis of 2-[2-oxo-2-(propan-2-yloxy)ethyl]-4-(trifluoromethyl)pyridine-3-carboxylic acid

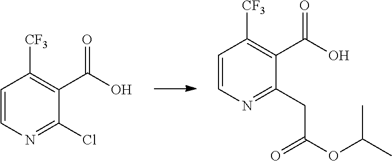 [SMILES: O=C(O)c1c(C(F)(F)F)ccnc1Cl ; CC(C)OC(=O)Cc1nccc(C(F)(F)F)c1C(=O)O]

To a solution of potassium tert-butoxide (7.2 g, 63.8 mmol) in 2-propanol (80 mL) was added ethyl acetoacetate (5.4 mL, 42.6 mmol) at room temperature. The reaction mixture was stirred for 30 minutes. Copper(II) acetate (390 mg, 2.13 mmol) and 2-chloro-4-(trifluoromethyl)pyridine-3-carboxylic acid (4.8 g, 21.3 mmol) were added and reaction mixture was heated to 80° C. for 16 hours. The reaction mixture was acidified with acetic acid and the volatiles were removed under reduce pressure. The residue was poured into water and the products were extracted with ethyl acetate. The combined organic layers were dried over anhydrous sodium sulfate and concentrated. The crude material was purified by reverse phase column chromatography eluting with 5-100% acetonitrile in water gradient to give 2-[2-oxo-2-(propan-2-yloxy)ethyl]-4-(trifluoromethyl)pyridine-3-carboxylic acid (3.6 g, 11.3 mmol, 53% yield) as a brown solid. 1H NMR (400 MHz, DMSO-d6) δ 14.34 (s, 1H), 8.87 (d, 1H), 7.80 (d, 1H), 4.90 (hept, 1H), 3.95 (s, 2H), 1.17 (d, 6H). LCMS (Method 4-Column 8): Retention Time=3.51 minutes, [MH]+=292.

### Synthesis of 4-(trifluoromethyl)-5,6,7,8-tetrahydro-1,6-naphthyridine-5,7-dione

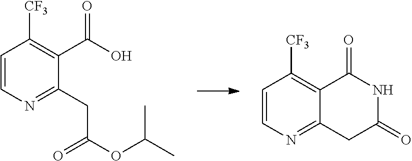 [SMILES: CC(C)OC(=O)Cc1nccc(C(F)(F)F)c1C(=O)O ; O=C1Cc2nccc(C(F)(F)F)c2C(=O)N1]

To a solution of 2-[2-oxo-2-(propan-2-yloxy)ethyl]-4-(trifluoromethyl)pyridine-3-carboxylic acid (3.0 g, 10.3 mmol) in tetrahydrofuran (30 mL) cooled to 0° C., was added triethylamine (2.2 mL, 15.4 mmol) and the reaction was stirred for 20 minutes. Ethyl chloroformate (1.5 mL, 15.4 mmol) was added dropwise maintaining the temperature at 0° C. and the reaction was stirred at room temperature for 4 days. The reaction mixture was quenched by addition of a saturated sodium bicarbonate solution and the products were extracted with ethyl acetate. The combined organic layers were dried over anhydrous sodium sulfate and concentrated under reduced pressure. The resulting intermediate was dissolved in tetrahydrofuran (45 mL) and the reaction mixture was cooled to 0° C. and ammonium hydroxide (25-30% in water, 25.7 mL, 164.8 mmol) was added dropwise. The reaction was stirred at room temperature for a further 2 hours. The reaction mixture was diluted with water and the aqueous layer was washed with ethyl acetate, which was discarded. The aqueous layer was then neutralised with dilute aqueous hydrochloric acid solution and the products were extracted with ethyl acetate, the combined organics were dried over anhydrous sodium sulfate and the concentrated under reduced pressure to give 4-(trifluoromethyl)-5,6,7,8-tetrahydro-1,6-naphthyridine-5,7-dione (1.7 g, 7.2 mmol, 69% yield) as a brick red solid. 1H NMR (400 MHz, DMSO-d6) δ 11.59 (s, 1H), 9.02 (d, 1H), 7.88 (d, 1H), 4.17 (s, 2H). LCMS (Method 4-Column 7): Retention Time=1.20 minutes, [MH]+=231.

### Synthesis of 5,7-dichloro-4-(trifluoromethyl)-1,6-naphthyridine

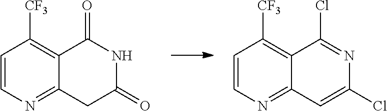 [SMILES: O=C1Cc2nccc(C(F)(F)F)c2C(=O)N1 ; FC(F)(F)c1ccnc2cc(Cl)nc(Cl)c12]

To a mixture of 4-(trifluoromethyl)-5,6,7,8-tetrahydro-1,6-naphthyridine-5,7-dione (1.6 g, 6.7 mmol) and tetramethylammonium chloride (770 g, 7.0 mmol) was added phosphoryl chloride (8.1 mL, 97.1 mmol). The reaction mixture was heated to 130° C. for 24 hours. The cooled reaction mixture was concentrated under reduce pressure and azeotroped with toluene. The residue was poured into cold water, basified with saturated aqueous sodium bicarbonate solution and the products were extracted with ethyl acetate. The combined organic layer was dried over anhydrous sodium sulfate and concentrated. The crude material was purified by column chromatography on silica gel eluting with an ethyl acetate in hexane gradient to give 5,7-dichloro-4-(trifluoromethyl)-1,6-naphthyridine (960 mg, 3.6 mmol, 54% yield) as a light orange solid. 1H NMR (400 MHz, DMSO-d6) δ 9.40 (d, 1H), 8.35 (s, 1H), 8.28 (d, 1H). LCMS (Method 4-Column 7): Retention Time=2.38 minutes, [MH]+=268. HPLC: Retention Time=8.32 minutes.

### Synthesis of 5,7-dichloro-3-iodo-1,6-naphthyridine

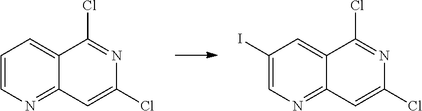 [SMILES: Clc1cc2ncccc2c(Cl)n1 ; Clc1cc2ncc(I)cc2c(Cl)n1]

To a solution of 5,7-dichloro-1,6-naphthyridine (200 mg, 1.0 mmol) in acetic acid (10 mL) was added N-iodosuccinimide (230 mg, 1.0 mmol) and the reaction mixture was heated to reflux for 24 hours. The reaction mixture was diluted with ethyl acetate (50 mL) and washed with an aqueous 2N sodium hydroxide solution followed by brine and then dried over anhydrous sodium sulfate and concentrated under reduced pressure. The crude material was purified by column chromatography on silica gel eluting with 0-50% ethyl acetate in heptane gradient to give 5,7-dichloro-3-iodo-1,6-naphthyridine (43 mg, 0.13 mmol, 13% yield) as a white solid. 1H NMR (400 MHz, Chloroform-d) δ 9.23 (d, 1H), 8.95 (dd, 1H), 7.90 (d, 1H). LCMS (Method 2): Retention Time=3.02 minutes, [MH]+=325.

### Synthesis of 5,7-dichloro-3-ethenyl-1,6-naphthyridine

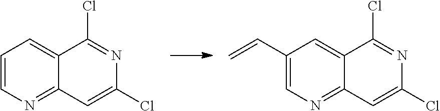

To a suspension of 5,7-dichloro-3-iodo-1,6-naphthyridine (43 mg, 0.13 mmol), 2,4,6-trivinylcyclotriboroxane pyridine complex (16 mg, 0.07 mmol) and potassium carbonate (35 mg, 1% 0.25 mmol) in 1,4-dioxane (1.5 mL) was added tetrakis(triphenylphosphine)palladium (15 mg, 0.01 mmol) and the reaction mixture was heated to 100° C. for 1 hour under microwave irradiation. The cooled reaction mixture was diluted with water and the products were extracted with ethyl acetate. The combined organic layer was washed with brine, dried over anhydrous sodium sulfate and concentrated under reduced pressure to give 5,7-dichloro-3-ethenyl-1,6-naphthyridine (23 mg, 0.10 mmol, 77% yield). 1H NMR (400 MHz, Chloroform-d) δ 9.20 (d, 1H), 8.44 (dd, 1H), 7.91 (d, 1H), 6.91 (dddd, 1H), 6.10 (d, 1H), 5.63 (d, 1H). LCMS (Method 2): Retention Time=2.85 minutes, [MH]+=225.

### Synthesis of 5,7-dichloro-3-methyl-1,6-naphthyridine

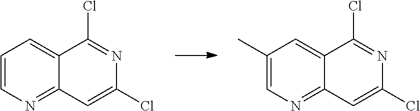

To a suspension of 5,7-dichloro-3-iodo-1,6-naphthyridine (137 mg, 0.42 mmol), trimethylboroxine (70 μL, 0.48 mmol) and potassium carbonate (120 mg, 0.87 mmol) in 1,4-dioxane (2 mL) was added tetrakis(triphenylphosphine)palladium (15 mg, 0.01 mmol). The reaction mixture was heated to 120° C. for 12 hours under microwave irradiation. The reaction mixture was partitioned between water and ethyl acetate, and the aqueous layer was further extracted with ethyl acetate. The combined organic layers were washed with brine, dried over anhydrous sodium sulfate and concentrated. The crude material was purified by column chromatography on silica gel eluting with 0-50% ethyl acetate in heptane gradient to give 5,7-dichloro-3-methyl-1,6-naphthyridine (68 mg. 0.32 mmol, 76% yield) as a white solid. LCMS (Method 2): Retention time=2.71 minutes, [MH]+=213.

### Synthesis of 4-bromo-2-methoxy-6-nitroaniline

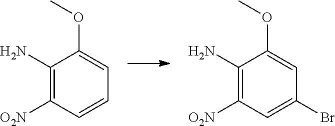

To a solution of 2-methoxy-6-nitroaniline (18.0 g, 107.0 mmol) in acetic acid (260 mL) were added sodium acetate (14.1 g, 171.3 mmol) and bromine (5.9 mL, 117.8 mmol) and the reaction was stirred at room temperature for 30 minutes. The resulting orange solid was collected by filtration and was washed with cold water and dried under vacuum to give 4-bromo-2-methoxy-6-nitroaniline (25.4 g, 102.8 mmol, 96% yield). 1H NMR (400 MHz, DMSO) δ 7.71 (d, 1H), 7.27 (s, 2H), 7.19 (d, 1H), 3.90 (s, 3H). LCMS (Method 4-Column 7): Retention Time=2.23 minutes, [MH]+=246/248.

### Synthesis of 5-bromo-3-methoxybenzene-1,2-diamine

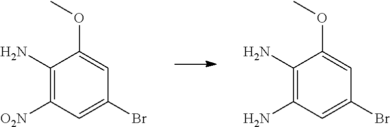

To a solution of 4-bromo-2-methoxy-6-nitroaniline (20.0 g, 81.0 mmol) in mixture of tetrahydrofuran (400 mL) and acetic acid (400 mL) was added portion-wise zinc dust (79.4 g, 1.2 mol) and the reaction was stirred at room temperature for 1 hour. The pH of the reaction mixture was adjusted to pH ˜7 by addition of an aqueous solution of 10% potassium carbonate. The resulting salts was removed by filtration and the filtrate was partitioned between water and ethyl acetate. The products were extracted with ethyl acetate. The combined organic layers were dried over anhydrous sodium sulfate and concentrated to give 5-bromo-3-methoxybenzene-1,2-diamine (18.0 g, 80.8 mmol, 100% yield) as a brown solid. 1H NMR (400 MHz, DMSO-d6) δ 6.40 (d, 1H), 6.34 (d, 1H), 4.78 (s, 2H), 4.11 (s, 2H), 3.71 (s, 3H). LCMS (Method 4-Column 7): Retention Time=1.41 minutes, [MH]+=217.

### Synthesis of 7-bromo-5-methoxyquinoxaline

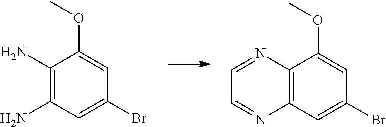

To a solution of 5-bromo-3-methoxybenzene-1,2-diamine (18.0 g, 82.9 mmol) in methanol (900 mL) was added glyoxal (40% solution in water, 36.1 g, 248.8 mmol). The reaction was stirred at room temperature for 16 hours. The volatiles were removed under reduce pressure and the crude material was purified by column chromatography on silica gel eluting with 300% ethyl acetate in n-hexane. The product was further purified by trituration with di-ethyl ether to give 7-bromo-5-methoxyquinoxaline (10.0 g, 41.8 mmol, 50% yield) as a light brown solid. 1H NMR (400 MHz, Chloroform-d) δ 8.86 (d, 1H), 8.83 (d, 1H), 7.89 (d, 1H), 7.20 (d, 1H), 4.11 (s, 3H). LCMS (Method 4-Column 7): Retention Time=1.58 minutes, [MH]+=239.

### Synthesis of 7-bromoquinoxalin-5-ol

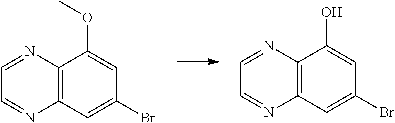

To a solution of 7-bromo-5-methoxyquinoxaline (10.0 g, 41.8 mmol) in dichloromethane (1.0 L) cooled to 0° C. was added boron tribromide (1 M in dichloromethane, 418 mL, 418.3 mmol) and the reaction was stirred at room temperature for 16 hours. The reaction mixture was poured into cold water and the aqueous layer was basified with saturated sodium bicarbonate solution. The products were extracted with dichloromethane, the combined organic layers were dried over anhydrous sodium sulfate and concentrated. The crude material was triturated with diethyl ether and dried under vacuum to give 7-bromoquinoxalin-5-ol (5.4 g, 24.0 mmol, 57% yield) as a light brown solid. 1H NMR (400 MHz, DMSO-d6) δ 11.05 (br s, 1H), 8.95 (s, 1H), 8.89 (s, 1H), 7.73 (s, 1H), 7.29 (s, 1H). LCMS (Method 4-Column 7): Retention Time=1.38 minutes, [MH]+=225. HPLC: Retention Time=3.79 minutes.

### Synthesis of 7-bromo-5-methoxyquinoline

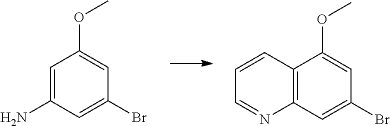

The reaction was performed in 8 batches of 10 g of 3-bromo-5-methoxyaniline each which were combined after work-up.

To a solution of glycerol (74 mL, 1 mol) in nitrobenzene (40 mL) were added 3-bromo-5-methoxyaniline (80 g, 0.4 mol) and sulfuric acid (14 M aqueous solution, 26 mL, 0.4 mol) and the reaction mixture was heated to 100° C. for 16 hours. The reaction was quenched by addition of ice and sodium hydroxide (6 M aqueous solution) and the products were extracted with ethyl acetate and then with 10% methanol in dichloromethane. The combined organic layers were dried over anhydrous sodium sulfate and concentrated. The crude material was purified by column chromatography on silica gel eluting with 15% ethyl acetate in n-hexane to give 7-bromo-5-methoxyquinoline (21.0 g, 81.1 mmol, 21% yield) as a yellow solid. 1H NMR (400 MHz, DMSO-d6) δ 8.95-8.89 (m, 1H), 8.49 (dd, 1H), 7.80 (d, 1H), 7.55 (dd, 1H), 7.22 (d, 1H), 4.02 (s, 3H) LCMS (Method 4-Column 2): Retention Time=2.08 minutes, [MH]+=238.

### Synthesis of 7-bromoquinolin-5-ol

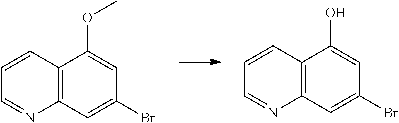

To a solution of 7-bromo-5-methoxyquinoline (21.0 g, 88.2 mmol) in dichloromethane (400 mL) cooled to 0° C., was added boron tribromide (1 M in dichloromethane, 882 mL, 882.1 mmol) and the reaction was stirred at room temperature for 1 day. The reaction mixture was cooled to 0° C. and quenched by addition of water and the pH of the mixture was adjusted by addition of solid sodium carbonate to pH=8-9. The products were extracted with butane 1-ol and then combined organic layers were concentrated. The crude material was purified by column chromatography on silica gel eluting with 6% methanol in dichloromethane to give 7-bromoquinolin-5-ol (13.5 g, 60.3 mmol, 68% yield) as a light brown solid. 1H NMR (400 MHz, DMSO-d6) δ 11.09 (s, 1H), 8.87 (dd, 1H), 8.47 (dd, 1H), 7.65 (d, 1H), 7.50 (dd, 1H), 7.04 (d, 1H). LCMS (Method 4-Column 7): Retention Time=1.47 minutes, [MH]+=224. HPLC: Retention Time=3.79 minutes.

### Synthesis of 7-bromo-5-methoxy-4-methylquinoline

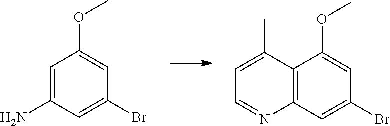

To a solution of 3-bromo-5-methoxyaniline (5.0 g, 24.8 mmol) in 1,4-dioxane (50 mL) was added concentrated sulfuric acid (2.0 mL, 37.1 mmol) and the mixture was heated to reflux for 30 minutes. Then a solution of but-3-en-2-one (2.6 g, 37.1 mmol) in 1,4-dioxane (5 mL) was added and the reaction mixture was heated at 100° C. for 4 hours. The reaction mixture was quenched by addition of an aqueous solution of sodium bicarbonate and the products were extracted with ethyl acetate. The combined organic layer was washed with water, brine, dried over anhydrous sodium sulphate and concentrated under reduced pressure. The crude material was purified by column chromatography on silica gel eluting with 10% ethyl acetate in petroleum ether to give 7-bromo-5-methoxy-4-methylquinoline (1.35 g, 5.35 mmol, 22% yield) as a brown solid. 1H NMR (400 MHz, Chloroform-d): δ 8.68 (d, 1H), 7.93 (d, 1H), 7.17-7.15 (m, 1H), 6.97 (d, 1H), 3.98 (s, 3H), 2.89 (d, 3H). LCMS (Method 5): Retention time=1.97 minutes, [MH]+=252.

### Synthesis of 7-bromo-4-methylquinolin-5-ol

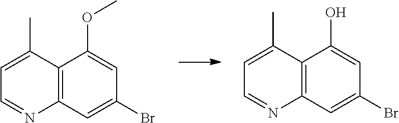

To a solution of 7-bromo-5-methoxy-4-methylquinoline (1.1 g, 4.4 mmol) in dichloromethane (20 mL) cooled to −78° C., was added boron tribromide (1.1 mL, 10.9 mmol) and the reaction was stirred at room temperature overnight. The reaction mixture was quenched with an aqueous solution of sodium bicarbonate and the products were extracted with ethyl acetate. The combined organic layer was washed with water, brine, dried over anhydrous sodium sulphate and concentrated under reduced pressure. The crude product was purified by trituration with n-hexane to give 7-bromo-4-methylquinolin-5-ol (520 mg, 2.2 mmol, 50% yield) as white solid. 1H NMR (400 MHz, DMSO-d6) δ 10.90 (s, 1H), 8.63 (d, 1H), 7.58 (d, 1H), 7.22 (d, 1H), 7.03 (d, 1H), 3.84 (s, 3H). LCMS (Method 5): Retention time=1.47 minutes, [MH]+=240.

### Synthesis of 7-(1-tert-butyl-1H-pyrazol-4-yl)-5-methoxyquinoline

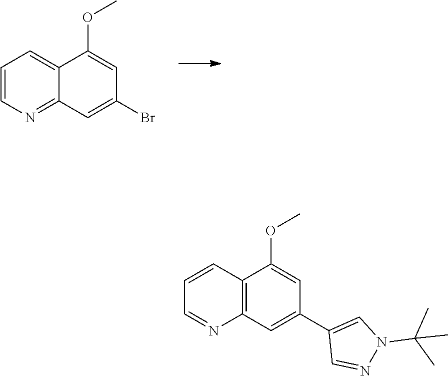 [SMILES: COc1cc(Br)cc2ncccc12 ; COc1cc(-c2cnn(C(C)(C)C)c2)cc2ncccc12]

To a solution of 7-bromo-5-methoxyquinoline (550 mg, 2.3 mmol) in 1,4-dioxane (10 mL) and water (2 mL) were added 1-t-butylpyrazole-4-boronic acid, pinacol ester (690 mg, 2.8 mmol) and sodium carbonate (730 mg, 6.9 mmol). The reaction mixture was purged with nitrogen gas for 30 minutes, followed by addition of [1,1′-bis(diphenylphosphino)ferrocene]dichloropalladium(II) (80 mg, 0.12 mmol). The reaction mixture was then heated to 80° C. for 3 hours. The reaction mixture was poured into water and the products were extracted with ethyl acetate. The combined organic layers were dried over anhydrous sodium sulfate and concentrated. The crude material was purified by column chromatography on silica gel eluting with 2% methanol in dichloromethane to give 7-(1-tert-butylpyrazol-4-yl)-5-methoxyquinoline (550 mg, 1.8 mmol, 78% yield) as a brown solid. 1H NMR (400 MHz, DMSO-d6) δ 8.84 (dd, 1H), 8.56 (d, 1H), 8.42 (ddd, 1H), 8.12 (d, 1H), 7.82 (s, 1H), 7.39 (dd, 1H), 7.33 (d, 1H), 4.07 (s, 3H), 1.59 (s, 9H). LCMS (Method 4-Column 2): Retention Time=1.66 minutes, [MH]+=282.

### Synthesis of 7-(1-tert-butyl-1H-pyrazol-4-yl)quinolin-5-ol

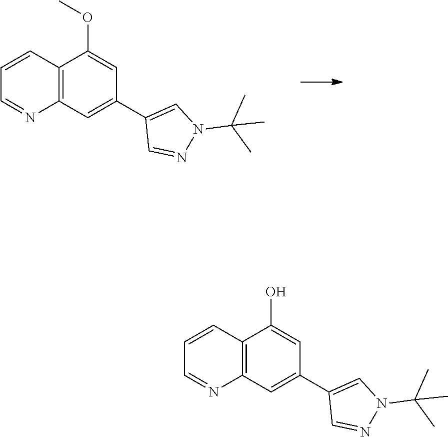 [SMILES: COc1cc(-c2cnn(C(C)(C)C)c2)cc2ncccc12 ; CC(C)(C)n1cc(-c2cc(O)c3cccnc3c2)cn1]

To a solution of 7-(1-tert-butyl-1H-pyrazol-4-yl)-5-methoxyquinoline (550 mg, 2.0 mmol) in dichloromethane (40 mL) cooled to 0° C. was added dropwise boron tribromide (1 M solution in dichloromethane, 39 mL, 39.1 mmol). The reaction mixture was warmed up to room temperature and stirring was continued for one day. The reaction mixture was quenched by dropwise addition of a 1 M aqueous sodium hydroxide solution (50 mL) to adjust the pH=8-9. The products were extracted with ethyl acetate. The combined organic layers were dried over anhydrous sodium sulfate and concentrated under reduced pressure. The resulting crude material was purified by column chromatography on silica eluting with 2% methanol in dichloromethane to give 7-(1-tert-butyl-1H-pyrazol-4-yl)quinolin-5-ol (320 mg, 1.2 mmol, 61% yield) as a brown solid. 1H NMR (400 MHz, DMSO-d6) δ 10.46 (s, 1H), 8.80 (dd, 1H), 8.41 (ddd, 1H), 8.38 (d, 1H), 7.94 (d, 1H), 7.71 (app t, 1H), 7.35 (dd, 1H), 7.15 (d, 1H), 1.58 (s, 9H). LCMS (Method 4-Column 2): Retention Time=1.43 minutes, [MH]+=268.

### 7-(1-tert-butyl-1H-pyrazol-4-yl)-5-{[(2S)-morpholin-2-yl]methoxy}quinoline (1)

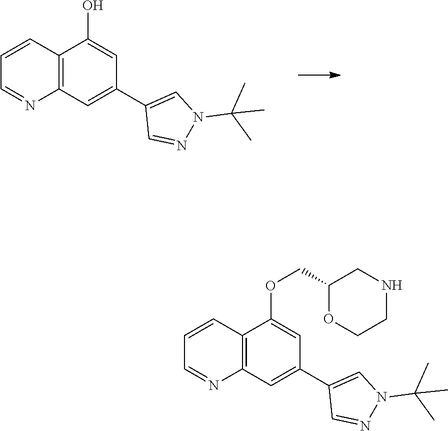 [SMILES: CC(C)(C)n1cc(-c2cc(O)c3cccnc3c2)cn1 ; CC(C)(C)n1cc(-c2cc(OC[C@@H]3CNCCO3)c3cccnc3c2)cn1]

To a solution of 7-(1-tert-butyl-1H-pyrazol-4-yl)quinolin-5-ol (93 mg, 0.26 mmol) in N,N-dimethylformamide (3.0 mL) cooled to 0° C. was added sodium hydride (57-63% w/w oil dispersion, 6 mg, 0.26 mmol). The reaction mixture was warmed to room temperature and stirred for 10 minutes and tert-butyl (2S)-2-(methylsulfonyloxymethyl)morpholine-4-carboxylate (50 mg, 0.17 mmol) was added and the reaction was stirred at room temperature for 2 hours. The reaction mixture was cooled in an ice bath and quenched by addition of water. The reaction mixture was partitioned between ethyl acetate and water. The products were extracted with ethyl acetate and the combined organic layers were washed with brine, dried over anhydrous sodium sulfate and concentrated.

To a solution of the crude intermediate in dichloromethane (5 mL) was added trifluoroacetic acid (0.5 mL) and the reaction mixture was stirred at room temperature for 1 hour. The reaction mixture was concentrated, and the residue was purified by prep-HPLC (purification method 2) to give 7-(1-tert-butyl-1H-pyrazol-4-yl)-5-{[(2S)-morpholin-2-yl]methoxy}quinoline (38 mg, 0.99 mmol, 38% yield) as a colourless solid. 1H NMR (400 MHz, DMSO-d6) δ 8.84 (dd, 1H), 8.55 (d, 11H), 8.42 (ddd, 1H), 8.11 (d, 1H), 7.82 (app t, 1H), 7.41 (dd, 1H), 7.33 (d, 1H), 4.30-4.15 (m, 2H), 3.87 (dddd, 1H), 3.84-3.77 (m, 1H), 3.55 (ddd, 1H), 3.02 (dd, 1H), 2.77-2.62 (m, 3H), 1.59 (s, 9H). LCMS (Method 2): Retention Time=1.51 minutes, [MH]+=367.

### Synthesis of 5-methoxy-7-(1-methyl-1H-pyrazol-4-yl)quinoline

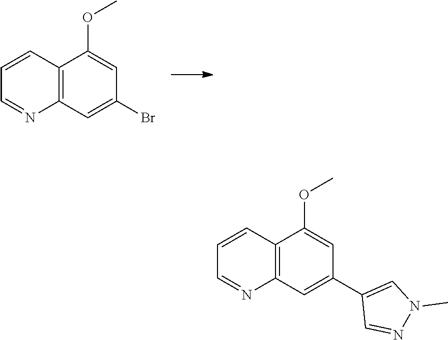 [SMILES: COc1cc(Br)cc2ncccc12 ; COc1cc(-c2cnn(C)c2)cc2ncccc12]

To a solution of 1-methyl-1H-pyrazole-4-boronic acid (630 mg, 5.0 mmol) and 7-bromo-5-methoxyquinoline (0.8 g, 3.4 mmol) in 1,4-dioxane (10 mL) was added potassium carbonate (1.2 g, 8.4 mmol). Nitrogen gas was bubbled through the reaction mixture for 15 minutes. After this time, [1,1′-bis(diphenylphosphino)ferrocene]palladium(II) dichloride (246 mg, 0.34 mmol) was added and the reaction mixture was heated to 70° C. for 16 hours. After this time, the reaction mixture was treated with water and the products were extracted with ethyl acetate. The combined organic layers were dried over anhydrous sodium sulfate and concentrated give 5-methoxy-7-(1-methyl-1H-pyrazol-4-yl)quinoline (720 mg, 2.6 mmol, 77% yield) as a red oil. 1H NMR (400 MHz, DMSO-d6) δ 8.84 (dd, 1H), 8.46-8.38 (m, 2H), 8.11 (s, 1H), 7.77 (s, 1H), 7.40 (dd, 1H), 7.27 (d, 1H), 4.05 (s, 3H), 3.91 (s, 3H). LCMS (Method 4-Column 2): Retention Time=1.35 minutes, [MH]+=240.

### Synthesis of 7-(1-methyl-1H-pyrazol-4-yl)quinolin-5-ol

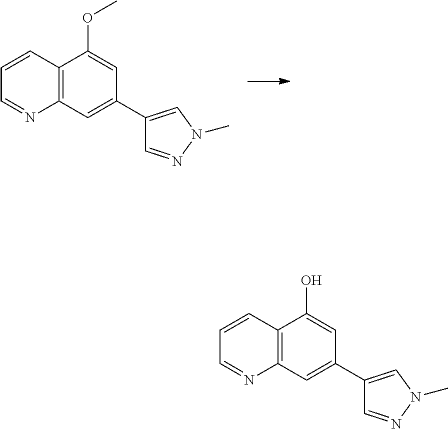 [SMILES: COc1cc(-c2cnn(C)c2)cc2ncccc12 ; Cn1cc(-c2cc(O)c3cccnc3c2)cn1]

To a solution of 5-methoxy-7-(1-methyl-1H-pyrazol-4-yl)quinoline (720 mg, 3.0 mmol) in dichloromethane (50 mL) cooled to 0° C., was added boron tribromide (1 M in dichloromethane, 60 mL, 60.1 mmol) and the reaction mixture was stirred at room temperature for 2 days. The reaction mixture was neutralised by the dropwise addition of sodium hydroxide (1 M aqueous solution) and the products were extracted with ethyl acetate. The combined organic layers were dried over anhydrous sodium sulfate and concentrated. The crude material was purified by column chromatography on silica gel eluting with 4% methanol in dichloromethane to give 74(1-methyl-1H-pyrazol-4-yl)quinolin-5-ol (370 mg, 1.6 mmol, 55% yield) as a light-brown solid. 1H NMR (400 MHz, DMSO-d6) δ 10.54 (s, 1H), 8.80 (dd, 1H), 8.42 (dd, 1H), 8.24 (s, 1H), 7.93 (s, 1H), 7.70-7.59 (m, 1H), 7.36 (dd, 1H), 7.10 (d, 1H), 3.90 (s, 3H). LCMS (Method 4-Column 2): Retention Time=1.21 minutes, [MH]+=226.

### 7-(1-methyl-1H-pyrazol-4-yl)-5-{[(2S)-morpholin-2-yl]methoxy}quinoline (2)

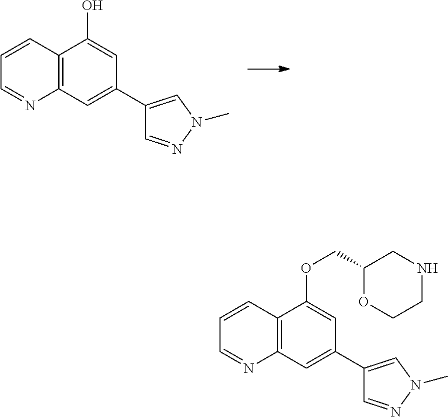 [SMILES: Cn1cc(-c2cc(O)c3cccnc3c2)cn1 ; Cn1cc(-c2cc(OC[C@@H]3CNCCO3)c3cccnc3c2)cn1]

To a solution of 7-(1-methyl-1H-pyrazol-4-yl)quinolin-5-ol (32 mg, 0.14 mmol) in N,N-dimethylformamide (2.5 mL) cooled to 0° C., was added sodium hydride (57-63% oil dispersion 3.4 mg, 0.14 mmol). The reaction mixture was warmed to room temperature before addition of tert-butyl (2S)-2-(methylsulfonyloxymethyl)morpholine-4-carboxylate (50 mg, 0.17 mmol). The reaction mixture was stirred at room temperature for 2 hours. The reaction mixture was cooled in an ice bath and quenched by addition of water and the products were extracted with ethyl acetate. The combined organic layer was washed with water, brine and dried over anhydrous sodium sulfate. The solvents were removed under reduced pressure to give the Boc-protected intermediate.

To a solution of the Boc-protected intermediate in dichloromethane (5 mL) and trifluoroacetic acid (0.5 mL) was added and the reaction mixture was stirred at room temperature for 1 hour. The volatiles were removed under reduced pressure and the crude material was purified by prep-HPLC to give 7-(1-methyl-1H-pyrazol-4-yl)-5-{[(2S)-morpholin-2-yl]methoxy}quinoline (30 mg, 0.09 mmol. 64% yield) as a colourless solid. 1H NMR (600 MHz, Methanol-d4) δ 8.80 (dd, 1H), 8.63 (dd, 1H), 8.19 (s, 1H), 8.02 (s, 1H), 7.75 (s, 1H), 7.45 (dd, 1H), 7.25 (s, 1H), 4.35 (dd, 1H), 4.32 (dd, 1H), 4.17-4.11 (m, 1H), 4.07 (dt, 1H), 3.98 (s, 3H), 3.83 (ddd, 1H), 3.35-3.32 (m, 1H), 3.11-3.03 (m, 3H). LCMS (Method 2): Retention Time=1.36 minutes, [MH]+=325.

### Synthesis of tert-butyl (2S)-2-[(1S)-1-{[7-(1-tert-butyl-1H-pyrazol-4-yl)quinolin-5-yl]oxy}ethyl]morpholine-4-carboxylate (Isomer 1) and tert-butyl (2S)-2-[(1R)-1-{[7-(1-tert-butyl-1H-pyrazol-4-yl)quinolin-5-yl]oxy}ethyl]morpholine-4-carboxylate (Isomer 2)

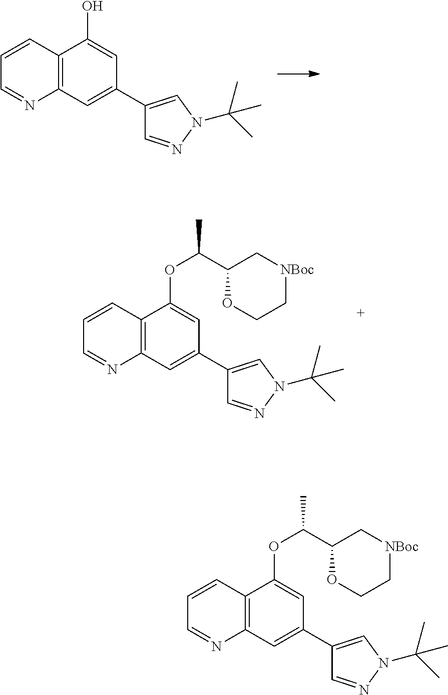 [SMILES: CC(C)(C)n1cc(-c2cc(O)c3cccnc3c2)cn1 ; C[C@H](Oc1cc(-c2cnn(C(C)(C)C)c2)cc2ncccc12)[C@@H]1CN(C(=O)OC(C)(C)C)CCO1 ; C[C@@H](Oc1cc(-c2cnn(C(C)(C)C)c2)cc2ncccc12)[C@@H]1CN(C(=O)OC(C)(C)C)CCO1]

To a suspension of sodium hydride (57-63% oil dispersion, 7 mg, 0.17 mmol) in acetonitrile (1.5 mL) were added 7-(1-tert-butylpyrazol-4-yl)quinolin-5-ol (30 mg, 0.11 mmol) and tert-butyl (2S)-2-(1-methylsulfonyloxyethyl)morpholine-4-carboxylate (52 mg, 0.17 mmol). The reaction mixture was stirred for 1 minute and a solution of sodium iodide (2 mg, 0.01 mmol) in acetonitrile (0.5 mL) was then added. The reaction mixture was stirred at room temperature for 5 minutes before heating under microwave irradiation at 130° C. for 2 hours. The cooled reaction mixture was quenched by slow addition of water and the reaction mixture was partitioned between water and ethyl acetate. The aqueous layer was further extracted with ethyl acetate and the combined organic layers were washed with brine, dried over anhydrous magnesium sulfate and concentrated to give a brown oil. The crude material was purified by column chromatography on silica gel eluting with 5-40% ethyl acetate in heptane gradient to give a mixture of diastereoisomers. The diastereoisomers were separated by prep-HPLC to give Isomer 1: tert-butyl (2S)-2-[(1S)-1-[7-(1-tert-butylpyrazol-4-yl)quinolin-5-yl]oxyethyl]morpholine-4-carboxylate (7 mg, 0.01 mmol, 11% yield) as a pale yellow residue and Isomer 2: tert-butyl (2S)-2-[(1R)-1-[7-(1-tert-butylpyrazol-4-yl)quinolin-5-yl]oxyethyl]morpholine-4-carboxylate (11 mg, 0.02 mmol, 19% yield) as a pale yellow residue.

### tert-butyl (2S)-2-[(1S)-1-{[7-(1-tert-butyl-1H-pyrazol-4-yl)quinolin-5-yl]oxy}ethyl]morpholine-4-carboxylate

1H NMR (400 MHz, Methanol-d4) δ 8.77 (dd, 1H), 8.62 (d, 1H), 8.36 (d, 1H), 8.04 (d, 1H), 7.74 (app t, 1H), 7.41 (dd, 1H), 7.36-7.32 (m, 1H), 5.01-4.91 (m, 1H), 4.06 (d, 1H), 3.96 (d, 1H), 3.91-3.83 (m, 1H), 3.70 (ddd, 1H), 3.57 (td, H), 3.08-2.90 (m, 2H), 1.66 (s, 9H), 1.46-1.39 (m, 12H). LCMS (Method 2): Retention Time=2.73 minutes, [MH]+=481.

### tert-butyl (2S)-2-[(1R)-1-{[7-(1-tert-butyl-1H-pyrazol-4-yl)quinolin-5-yl]oxy}ethyl]morpholine-4-carboxylate

1H NMR (400 MHz, Methanol-d4) δ 8.77 (dd, 1H), 8.60 (d, 1H), 8.37 (d, 1H), 8.04 (d, 1H), 7.75 (app t, 1H), 7.42 (dd, 1H), 7.35 (dd, 1H), 4.92-4.85 (m, 1H), 4.35-4.03 (m, 1H), 3.94 (d, 1H), 3.90-3.73 (m, 1H), 3.70-3.53 (m, 2H), 3.04 (ddd, 2H), 1.65 (s, 9H), 1.53-1.34 (m, 12H). LCMS (Method 2): Retention Time=2.87 minutes, [MH]+=481.

### Synthesis of tert-butyl (2S)-2-({[7-(4,4,5,5-tetramethyl-1,3,2-dioxaborolan-2-yl)quinolin-5-yl]oxy}methyl)morpholine-4-carboxylate

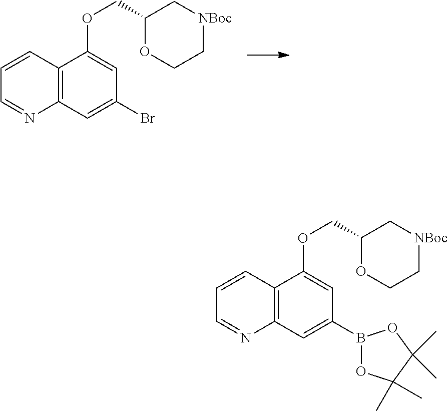 [SMILES: CC(C)(C)OC(=O)N1CCO[C@H](COc2cc(Br)cc3ncccc23)C1 ; CC(C)(C)OC(=O)N1CCO[C@H](COc2cc(B3OC(C)(C)C(C)(C)O3)cc3ncccc23)C1]

To a microwave vial was added 1,1′-bis(diphenylphosphino)ferrocene-palladium(II) dichloride (10 mg, 0.01 mmol), potassium acetate (84 mg, 0.85 mmol) and bis(pinacolato)diboron (79 mg, 0.31 mmol). The vial was sealed and placed under vacuum and backfilled with nitrogen. This was repeated twice more before addition of a degassed solution of tert-butyl (2S)-2-[(7-chloroquinolin-5-yl)oxymethyl]morpholine-4-carboxylate (120 mg, 0.28 mmol) in 1,4-dioxane (4 mL). The reaction mixture was heated at 130° C. for 1 hour under microwave irradiation. The cooled reaction mixture was partitioned between water and ethyl acetate. The aqueous layer was extracted with ethyl acetate and the combined organic layers were washed with brine, dried over anhydrous magnesium sulfate and concentrated under reduced pressure. The crude material was purified by column chromatography on silica gel eluting with 0-100% (2:24:74 aqueous ammonia:ethanol:ethyl acetate mix) in ethyl acetate gradient to give tert-butyl (2S)-2-({[7-(4,4,5,5-tetramethyl-1,3,2-dioxaborolan-2-yl)quinolin-5-yl]oxy}methyl)morpholine-4-carboxylate (131 mg, 0.21 mmol, 74% yield) as a beige residue, impurities still present. Intermediate used in the next step without further purification. 1H NMR (400 MHz, Chloroform-d) δ 8.93 (d, 1H), 8.61 (d, 1H), 8.24 (s, 1H), 7.41 (dd, 1H), 7.20 (s, 1H), 4.31 (dd, 1H), 4.27-4.06 (m, 2H), 4.01-3.82 (m, 3H), 3.65 (td, 1H), 3.11-2.86 (m, 2H), 1.49 (s, 9H), 1.38 (s, 12H). LCMS (Method 2): Retention Time=2.08 minutes, [(M−82)H]+=389.

### Synthesis tert-butyl (2S)-2-[({7-[5-(difluoromethyl)thiophen-3-yl]quinolin-5-yl)oxy}methyl]morpholine-4-carboxylate

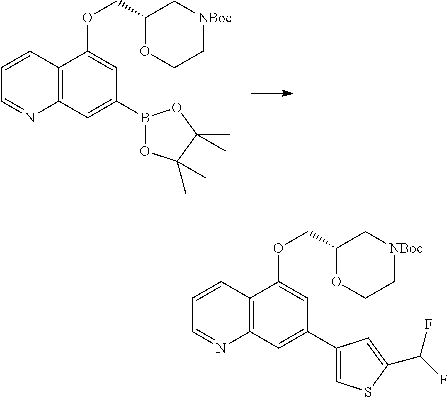 [SMILES: CC(C)(C)OC(=O)N1CCO[C@H](COc2cc(B3OC(C)(C)C(C)(C)O3)cc3ncccc23)C1 ; CC(C)(C)OC(=O)N1CCO[C@H](COc2cc(-c3csc(C(F)F)c3)cc3ncccc23)C1]

To a microwave vial was added 4-bromo-2-(difluoromethyl)thiophene (25 mg, 0.12 mmol), 1,1′-bis(diphenylphosphino)ferrocene-palladium(II) dichloride (4 mg, 0.01 mmol), and potassium acetate (35 mg, 0.35 mmol). The vial was sealed and evacuated and back filled with nitrogen. This was repeated twice more before addition of a degassed solution of tert-butyl (2S)-2-[[7-(4,4,5,5-tetramethyl-1,3,2-dioxaborolan-2-yl)quinolin-5-yl]oxymethyl]morpholine-4-carboxylate (40 mg, 0.06 mmol), in 1,4-dioxane (1 mL). The reaction was heated under microwave irradiation at 130° C. for 1 hour. The cooled reaction mixture was partitioned between water and ethyl acetate. The products were extracted with ethyl acetate and the combined organic layers were washed with brine, dried over anhydrous magnesium sulfate and concentrated to give a brown residue. The residue was purified by column chromatography on silica gel eluting with 5-500% ethyl acetate in heptane gradient to give tert-butyl (2S)-2-[({7-[5-(difluoromethyl)thiophen-3-yl]quinolin-5-yl}oxy)methyl]morpholine-4-carboxylate (18 mg, 0.04 mmol, 32% yield) as a pale brown residue. Intermediate used in the next step without further purification. 1H NMR (400 MHz, Methanol-d4) δ 8.84 (d, 1H), 8.66 (d, 1H), 8.10 (s, 1H), 7.92 (d, 1H), 7.86 (s, 1H), 7.49 (dd, 1H), 7.36 (d, 1H), 7.09 (t, 1H), 4.38 (dd, 1H), 4.32 (dd, 1H), 4.22-4.11 (m, 1H), 4.01-3.93 (m, 2H), 3.89 (d, 1H), 3.63 (td, 1H), 3.16-2.88 (m, 2H), 1.47 (s, 9H). LCMS (Method 2): Retention Time=3.03 minutes, [MH]+=477.

### Synthesis of tert-butyl (2S)-2-[({7-[4-(difluoromethyl)thiophen-2-yl]quinolin-5-yl}oxy)methyl]morpholine-4-carboxylate

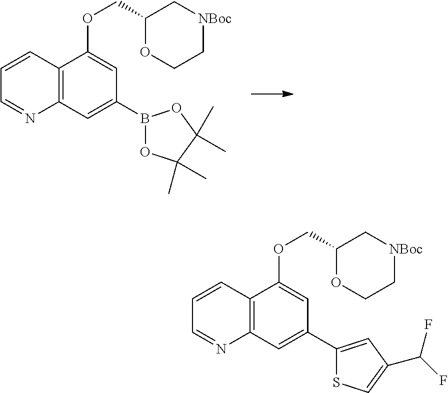 [SMILES: CC(C)(C)OC(=O)N1CCO[C@H](COc2cc(B3OC(C)(C)C(C)(C)O3)cc3ncccc23)C1 ; CC(C)(C)OC(=O)N1CCO[C@H](COc2cc(-c3cc(C(F)F)cs3)cc3ncccc23)C1]

To a microwave vial was added 2-bromo-4-(difluoromethyl)thiophene (18 mg, 0.09 mmol), 1,1′-bis(diphenylphosphino)ferrocene-palladium(II) dichloride (3 mg, 0.004 mmol), and potassium acetate (25 mg, 0.26 mmol). The vial was sealed and evacuated and back filled with nitrogen, this was repeated twice more before addition of a solution of a degassed solution of tert-butyl (2S)-2-({[7-(4,4,5,5-tetramethyl-1,3,2-dioxaborolan-2-yl)quinolin-5-yl]oxy}methyl)morpholine-4-carboxylate (40 mg, 0.09 mmol) in 1,4-dioxane (1 mL)/water (0.1 mL). The reaction was heated at 130° C. under microwave irradiation for 1 hour. The cooled reaction mixture was filtered through a pad of Celite® and the filtrate was partitioned between water and ethyl acetate. The aqueous layer was further extracted with ethyl acetate and the combined organic layers were washed with brine, dried over anhydrous magnesium sulfate and concentrated to give a brown residue. The residue was purified by column chromatography on silica gel eluting with 0-50% ethyl acetate in heptane gradient to give tert-butyl (2S)-2-[({7-[4-(difluoromethyl)thiophen-2-yl]quinolin-5-yl}oxy)methyl]morpholine-4-carboxylate (7.0 mg, 0.01 mmol, 12% yield) as a light brown residue, sample taken through to next step without purification. 1H NMR (400 MHz, Chloroform-d) δ 8.96-8.88 (m, 1H), 8.56 (d, 1H), 7.96 (s, 1H), 7.58-7.52 (m, 2H), 7.41-7.32 (m, 1H), 7.09 (s, 1H), 6.71 (t, 1H), 4.32 (dd, 1H), 4.27-4.11 (m, 2H), 4.01-3.88 (m, 3H), 3.70-3.61 (m, 1H), 3.10-2.85 (m, 2H), 1.49 (s, 9H). LCMS (Method 2): Retention Time=3.10 minutes, [MH]+=477.

### Synthesis of tert-butyl (2S)-2-({[7-(5-tert-butylthiophen-2-yl)quinolin-5-yl]oxy}methyl)morpholine-4-carboxylate

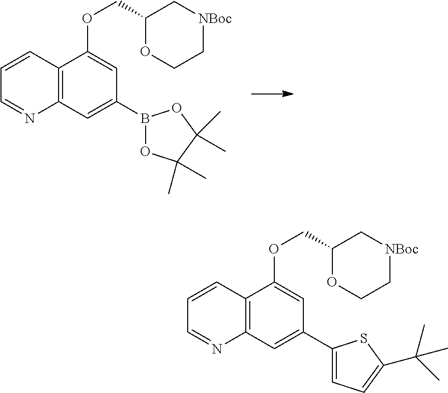 [SMILES: CC(C)(C)OC(=O)N1CCO[C@H](COc2cc(B3OC(C)(C)C(C)(C)O3)cc3ncccc23)C1 ; CC(C)(C)OC(=O)N1CCO[C@H](COc2cc(-c3ccc(C(C)(C)C)s3)cc3ncccc23)C1]

To a microwave vial was added 1,1′-bis(diphenylphosphino)ferrocene-palladium(II) dichloride (3 mg, 0.004 mmol), 2-bromo-5-tert-butylthiophene (19 mg, 0.09 mmol) and potassium acetate (25 mg, 0.26 mmol). The vial was sealed and evacuated and back filled with nitrogen, this was repeated twice more before addition of a degassed solution of a solution of tert-butyl (2S)-2-({[7-(4,4,5,5-tetramethyl-1,3,2-dioxaborolan-2-yl)quinolin-5-yl]oxy)}methyl)morpholine-4-carboxylate (40 mg, 0.09 mmol) in 1,4-dioxane (1 mL)/water (0.1 ML). The reaction was heated at 130° C. under microwave irradiation for 45 minutes. The cooled reaction mixture was filtered through a pad of Celite® and the filtrate was partitioned between water and ethyl acetate. The aqueous layer was further extracted with ethyl acetate and the combined organic layers were washed with brine, dried over anhydrous magnesium sulfate and concentrated to give a brown residue. The crude material was purified by column chromatography on silica gel eluting with 0-50% ethyl acetate in heptane gradient to give intermediate tert-butyl (2S)-2-({[7-(5-tert-butylthiophen-2-yl)quinolin-5-yl]oxy}methyl)morpholine-4-carboxylate (10 mg, 0.02 mmol, 17% yield) as a light brown residue. The intermediate was used without further purification in the next step. 1H NMR (400 MHz, Chloroform-d) δ 8.87 (dd, 1H), 8.52 (d, 1H), 7.93 (s, 1H), 7.35-7.29 (m, 2H), 7.08 (d, 1H), 6.86 (d, 1H), 4.31 (dd, 1H), 4.26-4.16 (m, 2H), 4.02-3.86 (m, 3H), 3.65 (td, 1H), 3.12-2.87 (m, 2H), 1.49 (s, 9H), 1.43 (s, 9H). LCMS (Method 2): Retention Time=3.49 minutes, [MH]+=483.

### Synthesis of 6-(5-{[(2S)-morpholin-2-yl]methoxy}quinolin-7-yl)-2,3-dihydro-1H-pyrrolizin-1-one (3)

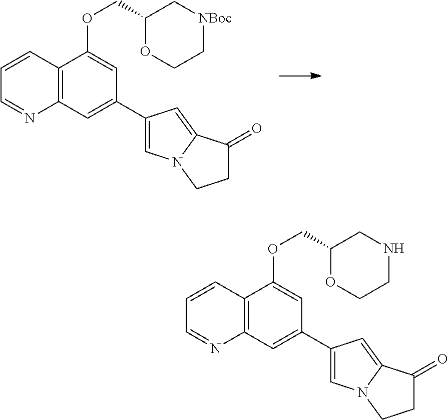 [SMILES: CC(C)(C)OC(=O)N1CCO[C@H](COc2cc(-c3cc4n(c3)CCC4=O)cc3ncccc23)C1 ; O=C1CCn2cc(-c3cc(OC[C@@H]4CNCCO4)c4cccnc4c3)cc21]

To a solution of tert-butyl (2S)-2-[[7-(7-oxo-5,6-dihydropyrrolizin-2-yl)quinolin-5-yl]oxymethyl]morpholine-4-carboxylate (50 mg, 0.11 mmol) in dichloromethane (0.9 mL) was added triethylsilane (50 μL, 0.31 mmol) and trifluoroacetic acid (0.1 mL, 1.3 mmol). The reaction mixture was stirred at room temperature for 15 hours. The reaction mixture was diluted with dichloromethane and the products were extracted with 1 M aqueous solution of hydrochloric acid. The combined aqueous layers were neutralised with sodium hydroxide (10 mL of a 2 M aqueous solution) and the products were extracted with dichloromethane. The combined organic layers were washed with brine, dried over anhydrous sodium sulfate and concentrated to give 6-(5-{([(2S)-morpholin-2-yl]methoxy}quinolin-7-yl)-2,3-dihydro-1H-pyrrolizin-1-one (17 mg, 0.05 mmol, 43% yield) as a white solid. 1H NMR (600 MHz, Methanol-d4) δ 8.78 (dd, 1H), 8.61 (dd, 1H), 7.85 (s, 1H), 7.77 (s, 1H), 7.44 (dd, 1H), 7.25 (d, 1H), 7.18 (d, 1H), 4.43 (t, 2H), 4.28 (dd, 1H), 4.24 (dd, 1H), 4.04-3.99 (m, 1H), 3.98-3.92 (m, 1H), 3.73 (td, 1H), 3.15-3.07 (m, 3H), 2.93-2.80 (m, 3H). LCMS (Method 2): Retention Time=1.34 minutes, [MH]+=364.

### Synthesis of 7-(2,3-dihydro-1H-pyrrolizin-6-yl)-5-{[(2S)-morpholin-2-yl]methoxy}quinoline (4)

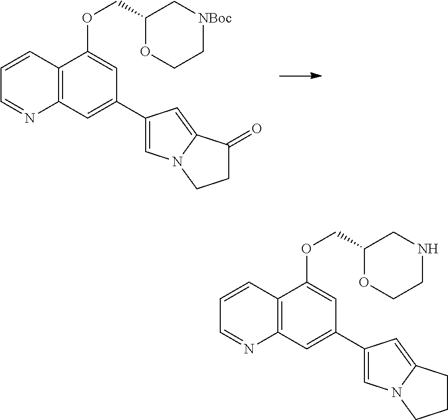 [SMILES: CC(C)(C)OC(=O)N1CCO[C@H](COc2cc(-c3cc4n(c3)CCC4=O)cc3ncccc23)C1 ; c1cnc2cc(-c3cc4n(c3)CCC4)cc(OC[C@@H]3CNCCO3)c2c1]

To a solution of tert-butyl (2S)-2-({[7-(1-oxo-2,3-dihydro-1H-pyrrolizin-6-yl)quinolin-5-yl]oxy}methyl)morpholine-4-carboxylate (50 mg, 0.11 mmol) in methanol (1 mL) was added sodium borohydride (15 mg, 0.40 mmol) and the reaction mixture was stirred at room temperature for 2 hours. The reaction mixture was quenched by addition of water and the products were extracted with dichloromethane. The combined organic layers were washed with brine, dried over anhydrous sodium sulfate and concentrated under reduced pressure to give the crude alcohol intermediate. To a solution of the crude intermediate in dichloromethane (2 mL), was added triethylsilane (0.1 mL, 0.63 mmol) and trifluoroacetic acid (0.1 mL, 1.3 mmol) and the reaction mixture was stirred at room temperature for 4 hours. The reaction mixture was partitioned between 1 M aqueous hydrochloric acid solution and dichloromethane. The aqueous layer was basified with sodium hydroxide (2 M aqueous solution) and the products were extracted with dichloromethane. The combined organic layers were washed with brine, dried over anhydrous sodium sulfate and concentrated under reduced pressure to give 7-(2,3-dihydro-1H-pyrrolizin-6-yl)-5-{[(2S)-morpholin-2-yl]methoxy)}quinoline (16 mg, 0.04 mmol, 38% yield) as a pale-yellow oil. 1H NMR (400 MHz, Methanol-d4) δ 8.71 (dd, 1H), 8.55 (ddd, 1H), 7.64 (app t, 1H), 7.35 (dd, 1H), 7.22 (d, 1H), 7.18 (d, 1H), 6.27-6.25 (m, 1H), 4.25 (dd, 1H), 4.20 (dd, 1H), 4.04-3.97 (m, 3H), 3.96-3.92 (m, 1H), 3.72 (ddd, 1H), 3.09 (dd, 1H), 2.90-2.82 (m, 5H), 2.57-2.45 (m, 2H). LCMS (Method 2): Retention Time=1.43 minutes, [MH]+=350.

### Synthesis of tert-butyl (2S)-2-[(1R)-1-{[7-(1H-pyrrol-3-yl)quinolin-5-yl]oxy}ethyl]morpholine-4-carboxylate

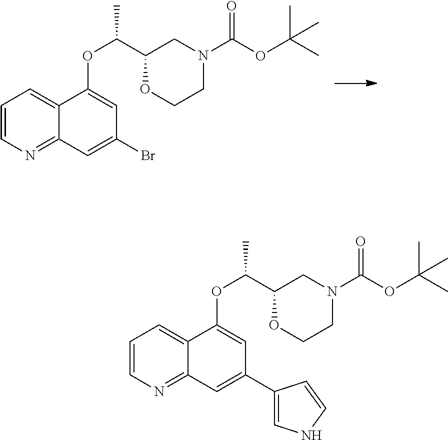 [SMILES: C[C@@H](Oc1cc(Br)cc2ncccc12)[C@@H]1CN(C(=O)OC(C)(C)C)CCO1 ; C[C@@H](Oc1cc(-c2cc[nH]c2)cc2ncccc12)[C@@H]1CN(C(=O)OC(C)(C)C)CCO1]

Pyrrole-3-boronic acid, pinacol ester (132 mg, 0.69 mmol), tert-butyl (2S)-2-[(1R)-1-(7-bromoquinolin-5-yl)oxyethyl]morpholine-4-carboxylate (250 mg, 0.57 mmol), potassium phosphate (364 mg, 1.7 mmol) and 1,1′-bis(diphenylphosphino)ferrocene-palladium(II) dichloride (42 mg, 0.06 mmol) were weighed into a 5 mL thick walled glass vial. Degassed 1,4-dioxane (2.8 mL) and water (0.7 mL) were added and the reaction mixture was heated at 100° C. for 1 hour. The cooled reaction mixture was diluted with ethyl acetate, dried over anhydrous sodium sulfate and concentrated under reduced pressure. The crude material was purified by column chromatography on silica gel eluting with 0-100% ethyl acetate in heptane gradient to give tert-butyl (2S)-2-[(1R)-1-{[7-(1H-pyrrol-3-yl)quinolin-5-yl]oxy}ethyl]morpholine-4-carboxylate (180 mg, 0.35 mmol, 61% yield) as a beige solid. LCMS (Method 2): Retention Time=2.17 minutes, [MH]+=424.

### Synthesis of rel-(1R,2S,5R)-2-(benzyloxy)-6-oxabicyclo[3.1.0]hexane and rel-(1R,2R,5R)-2-(benzyloxy)-6-oxabicyclo[3.1.0]hexane

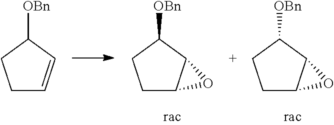 [SMILES: C1=CC(OCc2ccccc2)CC1 ; c1ccc(CO[C@@H]2CC[C@H]3O[C@@H]23)cc1 ; c1ccc(CO[C@H]2CC[C@H]3O[C@@H]23)cc1]

To a solution of cyclopent-2-en-1-yloxymethylbenzene (8.5 g, 48.8 mmol) in dichloromethane (50 mL) cooled to 0° C., was added portionwise 3-chloroperoxybenzoic acid (16.8 g, 97.6 mmol) and the reaction mixture was stirred at room temperature for 16 hours. The reaction mixture was diluted with dichloromethane and the organic layer was washed sequentially with 10% sodium sulfate solution, saturated aqueous sodium bicarbonate solution and water. The organic layer was dried over anhydrous sodium sulfate and was concentrated under reduced pressure. The crude material was purified by column chromatography on silica gel eluting with 6-8% ethyl acetate in hexane to give rel-(1R,2R,5R)-2-phenylmethoxy-6-oxabicyclo[3.1.0]hexane (3.8 g, 20.0 mmol, 41% yield) as a colourless liquid and rel-(1R,2S,5R)-2-phenylmethoxy-6-oxabicyclo[3.1.0]hexane (1.5 g, 7.9 mmol, 16% yield) as a light yellow liquid.

### rel-(1R,2R,5R)-2-phenylmethoxy-6-oxabicyclo[3.1.0]hexane

1H NMR (400 MHz, Chloroform-d) δ 7.40-7.29 (m, 4H), 7.32-7.24 (m, 1H), 4.60 (d, 1H), 4.52 (d, 1H), 4.10 (d, 1H), 3.55 (d, 1H), 3.49 (d, 1H), 1.98 (dd, 1H), 1.92-1.72 (m, 2H), 1.58-1.46 (m, 1H).

### rel-(1R,2S,5R)-2-phenylmethoxy-6-oxabicyclo[3.1.0]hexane

1H NMR (400 MHz, Chloroform-d) δ 7.42-7.27 (m, 5H), 4.64 (s, 2H), 4.05 (t, 1H), 3.50 (d, 1H), 3.43 (d, 1H), 2.10 (dd, 1H), 1.91-1.79 (m, 1H), 1.67-1.55 (m, 1H), 1.52-1.40 (m, 1H).

### Synthesis of rel-(1R,2S,5R)-2-azido-5-(benzyloxy)cyclopentan-1-ol

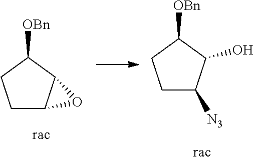 [SMILES: c1ccc(CO[C@@H]2CC[C@H]3O[C@@H]23)cc1 ; [N-]=[N+]=N[C@H]1CC[C@@H](OCc2ccccc2)[C@@H]1O]

To a solution of rel-(1R,2R,5R)-2-phenylmethoxy-6-oxabicyclo[3.1.0]hexane (3.8 g, 20.0 mmol) and ammonium chloride (2.5 g, 45.9 mmol) in methanol (8 mL):water (1 mL) was added sodium azide (6.5 g, 99.9 mmol) and the reaction mixture was heated to 80° C. for 16 hours. The cooled reaction mixture was concentrated under reduce pressure and the residue was partitioned between water and dichloromethane. The aqueous layer was further extracted with dichloromethane and the combined organic layers were dried over anhydrous sodium sulfate and concentrated under reduced pressure to give rel-(1R,2S,5R)-2-azido-5-phenylmethoxycyclopentan-1-ol (4.2 g, 18.0 mmol, 90% yield) as a brown liquid. 1H NMR (400 MHz, Chloroform-d) δ 7.42-7.27 (m, 5H), 4.59 (d, 1H), 4.53 (d, 1H), 3.97 (t, 1H), 3.84-3.70 (m, 1H), 3.69-3.56 (m, 1H), 2.50 (s, 1H), 2.09-1.93 (m, 2H), 1.85-1.68 (m, 2H).

### Synthesis of rel-(1R,2S,5S)-2-azido-5-(benzyloxy)cyclopentan-1-ol

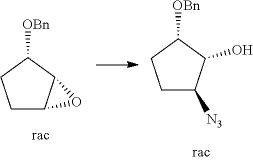 [SMILES: c1ccc(CO[C@H]2CC[C@H]3O[C@@H]23)cc1 ; [N-]=[N+]=N[C@H]1CC[C@H](OCc2ccccc2)[C@@H]1O]

To a solution of rel-(1R,2R,5R)-2-(benzyloxy)-6-oxabicyclo[3.1.0]hexane (2.0 g, 10.5 mmol) in mixture of methanol (8 mL) and water (1 mL) was added sodium azide (3.4 g, 52.6 mmol) and ammonium chloride (1.3 g, 24.2 mmol) and the reaction mixture was heated to 80° C. for 16 hours. The reaction mixture was concentrated under reduce pressure and the crude material was partitioned between water and dichloromethane and the aqueous layer was further extracted with dichloromethane. The combined organic layer was dried over anhydrous sodium sulfate and concentrated under reduced pressure to give rel-(1R,2S,5S)-2-azido-5-(benzyloxy)cyclopentan-1-ol (2.4 g, 9.3 mmol, 88% yield) as a brown liquid. 1H NMR (400 MHz, Chloroform-d) δ 7.41-7.28 (m, 5H), 4.62 (d, 1H), 4.49 (d, 1H), 4.01-3.92 (m, 1H), 3.92-3.79 (m, 2H), 2.74 (br s, 1H), 2.23-2.08 (m, 1H), 2.08-1.92 (m, 1H), 1.87-1.74 (m, 1H), 1.58-1.45 (m, 1H).

### Synthesis of rel-(1S,2S,5R)-2-azido-5-(benzyloxy)cyclopentyl 4-nitrobenzoate

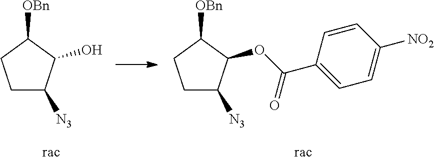 [SMILES: [N-]=[N+]=N[C@H]1CC[C@@H](OCc2ccccc2)[C@@H]1O ; [N-]=[N+]=N[C@H]1CC[C@@H](OCc2ccccc2)[C@H]1OC(=O)c1ccc([N+](=O)[O-])cc1]

To a solution of rel-(1R,2S,5R)-2-azido-5-phenylmethoxycyclopentan-1-ol (4.2 g, 18.0 mmol) in tetrahydrofuran (5 mL) was added 4-nitrobenzoic acid (0.54 g, 3.2 mmol) and triphenylphosphine (0.84 g, 3.2 mmol). The reaction mixture was cooled to 0° C. before addition of diisopropyl azodicarboxylate (0.69 g, 3.4 mmol). The reaction mixture was stirred at room temperature for 16 hours. The reaction mixture was poured in a saturated aqueous ammonium chloride solution and the products were extracted with ethyl acetate. The combined organic layers were dried over anhydrous sodium sulfate and concentrated under reduced pressure. The crude material was purified by column chromatography on silica gel eluting with 10% ethyl acetate in hexane to give rel-(1S,2S,5R)-2-azido-5-(benzyloxy)cyclopentyl 4-nitrobenzoate (2.4 g, 6.3 mmol, 35% yield) as a brown oil. 1H NMR (400 MHz, Chloroform-d) δ 8.31 (d, 2H), 8.18 (d, 2H), 7.38-7.26 (m, 5H), 5.37 (t, 1H), 4.65 (d, 1H), 4.60 (d, 1H), 4.05 (dd, 1H), 3.98-3.89 (m, 1H), 2.20-1.94 (m, 4H).

### Synthesis of rel-(1S,2S,5S)-2-azido-5-(benzyloxy)cyclopentyl 4-nitrobenzoate

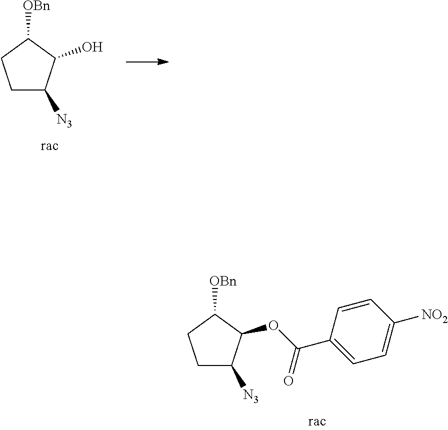 [SMILES: [N-]=[N+]=N[C@H]1CC[C@H](OCc2ccccc2)[C@@H]1O ; [N-]=[N+]=N[C@H]1CC[C@H](OCc2ccccc2)[C@H]1OC(=O)c1ccc([N+](=O)[O-])cc1]

To a solution of triphenylphosphine (5.4 g, 20.5 mmol) and 4-nitrobenzoic acid (3.6 g, 21.2 mmol) in tetrahydrofuran (70 mL) was added dropwise a solution of rel-(1R,2S,5S)-2-azido-5-(benzyloxy)cyclopentan-1-ol (3.3 g, 14.2 mmol) in tetrahydrofuran (20 mL) and the reaction mixture was stirred at room temperature for 15 minutes and the reaction mixture was cooled to 0° C. before dropwise addition of diisopropyl azodicarboxylate (4.6 g, 22.6 mmol). The reaction mixture was warmed to room temperature and stirred for 16 hours. The reaction mixture was partitioned between water and ethyl acetate and aqueous layer was further extracted with ethyl acetate. The combined organic layer was dried over anhydrous sodium sulfate and concentrated under reduced pressure. The crude material was purified by column chromatography on silica gel eluting with 5-15% ethyl acetate in hexane gradient to give rel-(1S,2S,5S)-2-azido-5-(benzyloxy)cyclopentyl 4-nitrobenzoate (3.0 g, 7.9 mmol, 56% yield) as a yellow liquid. 1H NMR (400 MHz, Chloroform-d) δ 8.35-8.27 (m, 2H), 8.22 (d, 2H), 7.41-7.28 (m, 5H), 5.39 (t, 1H), 4.62 (s, 2H), 4.22-4.08 (m, 2H), 2.37-2.11 (m, 2H), 2.02-1.86 (m, 1H), 1.86-1.69 (m, 1H).

### Synthesis of rel-(1S,2S,5R)-2-azido-5-(benzyloxy)cyclopentan-1-ol

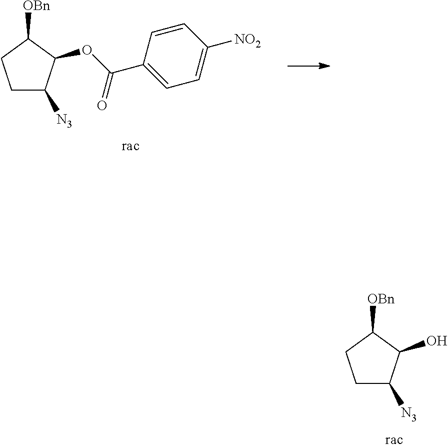 [SMILES: [N-]=[N+]=N[C@H]1CC[C@@H](OCc2ccccc2)[C@H]1OC(=O)c1ccc([N+](=O)[O-])cc1 ; [N-]=[N+]=N[C@H]1CC[C@@H](OCc2ccccc2)[C@H]1O]

To a solution of rel-(1S,2S,5R)-2-azido-5-(benzyloxy)cyclopentyl 4-nitrobenzoate (2.4 g, 6.3 mmol) in methanol (25 mL) was added potassium carbonate (2.6 g, 18.8 mmol) and the reaction mixture was stirred at room temperature for 16 hours. The solvents were removed under reduced pressure and the crude material was diluted with a saturated aqueous ammonium chloride solution. The products were extracted with ethyl acetate and the combined organic layer was dried over anhydrous sodium sulfate and concentrated under reduced pressure. The crude material was purified by column chromatography on neutral alumina eluting with 40% ethyl acetate in hexane to give rel-(1S,2S,5R)-2-azido-5-phenylmethoxycyclopentan-1-ol (1.4 g, 6.0 mmol, 96% yield) as a brown liquid. 1H NMR (400 MHz, Chloroform-d) δ 7.44-7.28 (m, 5H), 4.60 (d, 1H), 4.54 (d, 1H), 3.99 (t, 1H), 3.84-3.71 (m, 11H), 3.71-3.57 (m, 1H), 2.12-1.95 (m, 2H), 1.85-1.67 (m, 2H).

### Synthesis of rel-(1S,2S,5S)-2-azido-5-(benzyloxy)cyclopentan-1-ol

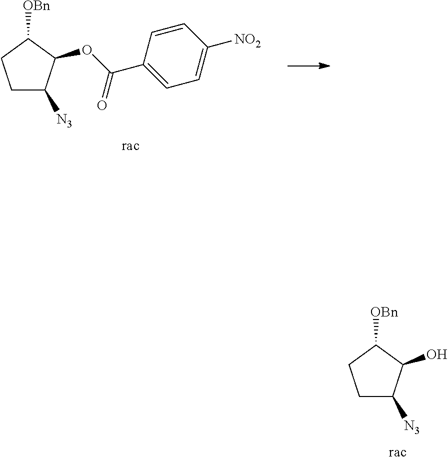 [SMILES: [N-]=[N+]=N[C@H]1CC[C@H](OCc2ccccc2)[C@H]1OC(=O)c1ccc([N+](=O)[O-])cc1 ; [N-]=[N+]=N[C@H]1CC[C@H](OCc2ccccc2)[C@H]1O]

To a stirred solution of rel-(1S,2S,5S)-2-azido-5-(benzyloxy)cyclopentyl 4-nitrobenzoate (3.0 g, 7.9 mmol) in methanol (30 mL) was added potassium carbonate (3.3 g, 23.5 mmol) and the reaction mixture was stirred at room temperature for 16 hours. The reaction mixture was concentrated under reduced pressure and the residue was partitioned between water and ethyl acetate. The aqueous layer was extracted with ethyl acetate and the combined organic layer was dried over anhydrous sodium sulfate and concentrated under reduce pressure. The crude material was purified by column chromatography on silica gel eluting with 0-30% ethyl acetate in hexane gradient to give rel-(1S,2S,5S)-2-azido-5-(benzyloxy)cyclopentan-1-ol (1.4 g, 5.8 mmol, 74% yield) as a light yellow liquid. 1H NMR (400 MHz, Chloroform-d) δ 7.38-7.27 (m, 5H), 4.55 (s, 2H), 4.10 (t, 1H), 4.06-3.99 (m, 1H), 3.92-3.83 (m, 1H), 2.26-2.04 (m, 2H), 1.88-1.76 (m, 1H), 1.73-1.62 (m, 1H).

### Synthesis of methyl 2-1[rel-(1S,2S,5R)-2-azido-5-(benzyloxy)cyclopentyl]oxy)acetate

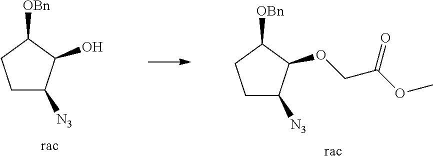 [SMILES: [N-]=[N+]=N[C@H]1CC[C@@H](OCc2ccccc2)[C@H]1O ; COC(=O)CO[C@H]1[C@@H](N=[N+]=[N-])CC[C@H]1OCc1ccccc1]

To a solution of rel-(1S,2S,5R)-2-azido-5-(benzyloxy)cyclopentan-1-ol (1.4 g, 6.0 mmol) in N,N-dimethylformamide (45 mL) cooled to 0° C., was added sodium hydride (60% dispersion in mineral oil, 1.9 g, 48.0 mmol). After stirring for 10 minutes was added methyl 2-bromoacetate (4.6 g, 30.0 mmol) and the reaction mixture was stirred at room temperature for 16 hours. The reaction mixture was quenched by slow addition of water and the products were extracted with diethyl ether. The combined organic layers were dried over anhydrous sodium sulfate and concentrated under reduced pressure. The crude material was purified by column chromatography, on silica gel, eluting with 0-20% ethyl acetate in hexane gradient to give methyl 2-{[rel-(1S,2S,5R)-2-azido-5-(benzyloxy)cyclopentyl]oxy}acetate (1.0 g, 3.3 mmol, 55% yield) as a yellow liquid. 1H NMR (400 MHz, Chloroform-d) δ 7.39-7.27 (m, 5H), 4.55 (d, 1H), 4.51 (d, 1H), 4.28 (d, 1H), 4.22 (d, 1H), 3.98-3.89 (m, 1H), 3.84-3.76 (m, 2H), 3.73 (s, 3H), 2.09-1.92 (m, 2H), 1.86-1.72 (m, 2H). LCMS (Method 4-Column 1): Retention Time=2.51 minutes, [MH]+=306.

### Synthesis of methyl 2-{[rel-(1S,2S,5S)-2-azido-5-(benzyloxy)cyclopentyl]oxy}acetate

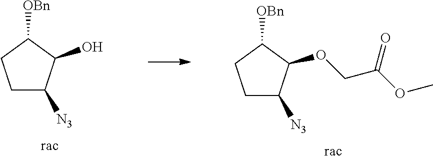 [SMILES: [N-]=[N+]=N[C@H]1CC[C@H](OCc2ccccc2)[C@H]1O ; COC(=O)CO[C@H]1[C@@H](N=[N+]=[N-])CC[C@@H]1OCc1ccccc1]

To solution of rel-(1S,2S,5S)-2-azido-5-(benzyloxy)cyclopentan-1-ol (1.4 g, 6.0 mmol) in N,N-dimethylformamide (50 mL) cooled to 0° C. was added portion-wise sodium hydride (60% dispersion in mineral oil, 1.9 g, 48.0 mmol). After stirring for 15 minutes was added dropwise methyl 2-bromoacetate (4.6 g, 30.0 mmol) and the reaction mixture was stirred at room temperature for 16 hours. The reaction mixture was quenched by slow addition of ice-cold water and the products were extracted with diethyl ether. Combined organic layer was dried over anhydrous sodium sulfate and concentrated under reduced pressure. The crude material was purified by column chromatography on silica gel eluting with 0-20% ethyl acetate in hexane to give methyl 2-{[rel-(1S,2S,5S)-2-azido-5-(benzyloxy)cyclopentyl]oxy}acetate (1.5 g, 4.9 mmol, 82% yield) as a light yellow liquid. 1H NMR (400 MHz, Chloroform-d) δ 7.40-7.27 (m, 5H), 4.55 (s, 2H), 4.32 (d, 1H), 4.24 (d, 1H), 4.13-4.04 (m, 1H), 4.00-3.90 (m, 2H), 3.74 (s, 3H), 2.25-2.13 (m, 1H), 2.08-1.95 (m, 1H), 1.86-1.73 (m, 1H), 1.69-1.60 (m, 1H). LCMS (Method 4-Column 1): Retention Time=2.56 mins, [MH]+=306.

### Synthesis of rel-(4aS,7R,7aS)-7-(benzyloxy)-octahydrocyclopenta[b][1,4]oxazin-3-one

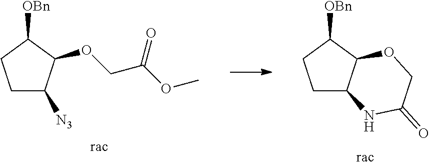 [SMILES: COC(=O)CO[C@H]1[C@@H](N=[N+]=[N-])CC[C@H]1OCc1ccccc1 ; O=C1CO[C@H]2[C@H](CC[C@H]2OCc2ccccc2)N1]

To a solution of methyl 2-{[rel-(1S,2S,5R)-2-azido-5-(benzyloxy)cyclopentyl]oxy}acetate (0.7 g, 2.3 mmol) in ethyl acetate (20 mL) was added palladium on carbon (10% w/w, 0.3 g, 0.28 mmol) and the reaction mixture was stirred at room temperature for 2 hours under an atmosphere of hydrogen. The reaction mixture was filtered through a bed of Celite® and the filtrated was concentrated under reduced pressure. The crude material was triturated with hexane (3 mL) to give rel-(4aS,7R,7aS)-7-(benzyloxy)-octahydrocyclopenta[b][1,4]oxazin-3-one (0.6 g, 2.4 mmol, 106% yield) as a light brown solid. 1H NMR (400 MHz, DMSO-d6) δ 8.38 (s, 1H), 7.42-7.22 (m, 5H), 4.58-4.45 (m, 2H), 4.13 (s, 2H), 3.95-3.87 (m, 1H), 3.72-3.52 (m, 2H), 2.22-2.03 (m, 1H), 1.83-1.58 (m, 2H), 1.58-1.35 (m, 1H). LCMS (Method 4-Column 9): Retention Time=1.67 minutes, [MH]+=248.

### Synthesis of rel-(4aS,7S,7aS)-7-(benzyloxy)-octahydrocyclopenta[b][1,4]oxazin-3-one

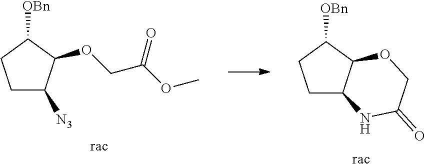 [SMILES: COC(=O)CO[C@H]1[C@@H](N=[N+]=[N-])CC[C@@H]1OCc1ccccc1 ; O=C1CO[C@H]2[C@H](CC[C@@H]2OCc2ccccc2)N1]

To a solution of methyl 2-{[rel-(1S,2S,5S)-2-azido-5-(benzyloxy)cyclopentyl]oxy}acetate (1.5 g, 4.9 mmol) in ethyl acetate (50 mL) was added palladium on carbon (10% w/w, 0.78 g, 0.73 mmol) and the reaction mixture was stirred at room temperature for 2 hours under an atmosphere of hydrogen. The reaction mixture was filtered through a Celite® plug, washing through with ethyl acetate. The filtrate was concentrated under reduce pressure and the crude material was triturated with n-hexane and dried under vacuum to give rel-(4aS,7S,7aS)-7-(benzyloxy)-octahydrocyclopenta[b][1,4]oxazin-3-one (1.1 g, 4.4 mmol, 89% yield) as a brown solid. 1H NMR (400 MHz, Chloroform-d) δ 7.42-7.26 (m, 5H), 6.39 (s, 1H), 4.54 (s, 2H), 4.18 (d, 1H), 4.08 (d, 1H), 4.01 (d, 1H), 3.98-3.93 (m, 1H), 3.82-3.73 (m, 1H), 2.32-2.19 (m, 1H), 2.18-2.08 (m, 1H), 1.82-1.62 (m, 2H). LCMS (Method 4-Column 1): Retention Time=1.84 minutes, [MH]+=248.

### Synthesis of rel-(4aS,7R,7aS)-7-(benzyloxy)-octahydrocydopenta[b][1,4]oxazine

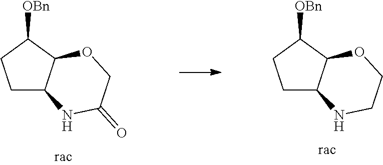 [SMILES: O=C1CO[C@H]2[C@H](CC[C@H]2OCc2ccccc2)N1 ; c1ccc(CO[C@@H]2CC[C@@H]3NCCO[C@@H]32)cc1]

To a solution of lithium aluminium hydride (1 M in tetrahydrofuran, 6.0 mL, 6.0 mmol) in tetrahydrofuran (30 mL) cooled to 0° C. was added dropwise a solution of rel-(4aS,7R,7aS)-7-(benzyloxy)-octahydrocyclopenta[b][1,4]oxazin-3-one (0.6 g, 2.4 mmol) in tetrahydrofuran (10 mL). The reaction mixture was warmed to room temperature and stirring was continued for 16 hours. The reaction mixture was quenched by slow addition of water and the products were extracted with ethyl acetate. The combined organic layers were dried over anhydrous sodium sulfate and concentrated under reduced pressure. The crude material was purified by trituration with hexane (5 mL) to give rel-(4aS,7R,7aS)-7-(benzyloxy)-octahydrocyclopenta[b][1,4]oxazine (470 mg, 2.0 mmol, 83% yield) as a light brown liquid. 1H NMR (400 MHz, DMSO-d6) δ 7.37-7.19 (m, 5H), 4.56-4.43 (m, 2H), 3.84-3.72 (m, 1H), 3.50-3.37 (m, 1H), 3.15-3.06 (m, 1H), 2.74-2.61 (m, 2H), 2.47-2.31 (m, 1H), 2.07-1.89 (m, 1H), 1.71-1.57 (m, 1H), 1.57-1.43 (m, 1H), 1.43-1.31 (m, 2H). LCMS (Method 9): Retention Time=2.25 minutes, [MH]+=234.

### Synthesis of rel-(4aS,7S,7aS)-7-(benzyloxy)-octahydrocyclopenta[b][1,4]oxazine

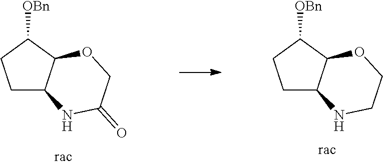 [SMILES: O=C1CO[C@H]2[C@H](CC[C@@H]2OCc2ccccc2)N1 ; c1ccc(CO[C@H]2CC[C@@H]3NCCO[C@@H]32)cc1]

To a solution of lithium aluminium hydride (1 M in tetrahydrofuran 8.9 mL, 8.9 mmol) in tetrahydrofuran (50 mL) cooled to 0° C. under nitrogen was added dropwise a solution of rel-(4aS,7S,7aS)-7-(benzyloxy)-octahydrocyclopenta[b][1,4]oxazin-3-one (1.1 g, 4.5 mmol) in tetrahydrofuran (10 mL) and the reaction mixture was stirred at room temperature for 16 hours. The reaction mixture was quenched by slow addition of water and the product were extracted with ethyl acetate. The combined organic layer was dried over anhydrous sodium sulfate and concentrated under educe pressure to give rel-(4aS,7S,7aS)-7-(benzyloxy)-octahydrocyclopenta[b][1,4]oxazine (1.0 g, 3.6 mmol, 82% yield) as a light brown liquid. 1H NMR (400 MHz, Chloroform-d) δ 7.40-7.26 (m, 5H), 4.58-4.49 (m, 2H), 3.97-3.88 (m, 1H), 3.88-3.82 (m, 1H), 3.82-3.73 (m, 1H), 3.54 (ddd, 1H), 3.42-3.33 (m, 1H), 3.05 (ddd, 1H), 2.67 (dt, 1H), 2.30-2.18 (m, 1H), 1.96-1.60 (m, 4H). LCMS (Method 4-Column 1): Retention Time=1.41 minutes, [MH]+=234.

### Synthesis of tert-butyl rel-(4aS,7R,7aS)-7-(benzyloxy)-octahydrocyclopenta[b][1,4]oxazine-4-carboxylate

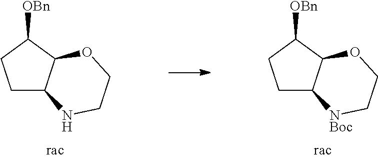 [SMILES: c1ccc(CO[C@@H]2CC[C@@H]3NCCO[C@@H]32)cc1 ; CC(C)(C)OC(=O)N1CCO[C@@H]2[C@H](OCc3ccccc3)CC[C@@H]21]

To a solution of rel-(4aS,7R,7aS)-7-(benzyloxy)-octahydrocyclopenta[b][1,4]oxazine (470 mg, 2.0 mmol) and diisopropylethylamine (780 mg, 6.0 mmol) in dichloromethane (10 mL) cooled to 0° C., di-tert-butyl dicarbonate (1.3 g, 6.0 mmol) and the reaction mixture was stirred at room temperature for 16 hours. The reaction mixture was partitioned between water and dichloromethane. The aqueous layer was further extracted with dichloromethane and the combined organic layers were dried over anhydrous sodium sulfate and concentrated under reduced pressure. The crude material was purified by column chromatography on silica gel, eluting with 10% ethyl acetate in hexane to give tert-butyl rel-(4aS,7R,7aS)-7-(benzyloxy)-octahydrocyclopenta[b][1,4]oxazine-4-carboxylate (370 mg, 1.1 mmol, 55% yield) as light yellow liquid. 1H NMR (400 MHz, DMSO-d6) δ 7.39-7.19 (m, 5H), 4.57-4.43 (m, 2H), 3.97-3.86 (m, 1H), 3.84-3.75 (m, 1H), 3.70 (d, 1H), 3.52 (td, 1H), 3.27 (dd, 1H), 2.90-2.74 (m, 2H), 2.28 (dd, 1H), 2.07-1.93 (m, 1H), 1.89-1.75 (m, 1H), 1.64-1.47 (m, 1H), 1.38 (s, 9H). LCMS (Method 4-Column 1): Retention Time=2.70 minutes, [MH]+=334.

### Synthesis of tert-butyl rel-(4aS,7S,7aS)-7-(benzyloxy)-octahydrocyclopenta[b][1,4]oxazine-4-carboxylate

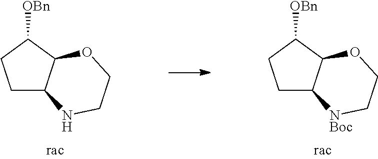 [SMILES: c1ccc(CO[C@H]2CC[C@@H]3NCCO[C@@H]32)cc1 ; CC(C)(C)OC(=O)N1CCO[C@@H]2[C@@H](OCc3ccccc3)CC[C@@H]21]

To a solution of rel-(4aS,7S,7aS)-7-(benzyloxy)-octahydrocyclopenta[b][1,4]oxazine (1.0 g, 4.3 mmol) in dichloromethane (25 mL) cooled to 0° C., was added diisopropylethylamine (1.7 g, 12.9 mmol) and the reaction mixture was stirred for 15 minute before addition of di-tert-butyl dicarbonate (2.8 g, 12.9 mmol). The reaction mixture was stirred at room temperature for 16 hours. The reaction was partitioned between water and dichloromethane. The aqueous layer was further extracted with dichloromethane and the combined organic layer was dried over anhydrous sodium sulfate and concentrated under reduced pressure. The crude material was purified by column chromatography on silica gel eluting with 0-20% ethyl acetate in hexane gradient to give tert-butyl rel-(4aS,7S,7aS)-7-(benzyloxy)-octahydrocyclopenta[b][1,4]oxazine-4-carboxylate (1.0 g, 3.0 mmol, 70% yield) as light yellow liquid. 1H NMR (400 MHz, Chloroform-d) δ 7.42-7.27 (m, 5H), 4.58-4.44 (m, 2H), 4.44-4.11 (m, 1H), 3.90-3.57 (m, 4H), 3.53-3.35 (m, 1H), 3.17-2.94 (m, 1H), 2.29-2.11 (m, 1H), 2.02-1.81 (m, 1H), 1.81-1.60 (m, 2H), 1.47 (s, 9H). LCMS (Method 4-Column 1): Retention Time=2.44 minutes, [(M-56)H]+=278.

### Synthesis of tert-butyl rel-(4aS,7R,7aS)-7-hydroxy-octahydrocyclopenta[b][1,4]oxazine-4-carboxylate

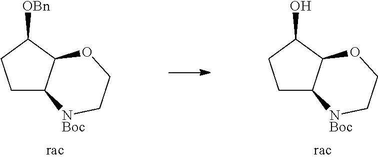 [SMILES: CC(C)(C)OC(=O)N1CCO[C@@H]2[C@H](OCc3ccccc3)CC[C@@H]21 ; CC(C)(C)OC(=O)N1CCO[C@@H]2[C@H](O)CC[C@@H]21]

To a solution of tert-butyl rel-(4aS,7R,7aS)-7-(benzyloxy)-octahydrocyclopenta[b][1,4]oxazine-4-carboxylate (370 mg, 1.1 mmol) in methanol (15 mL) was added palladium hydroxide on carbon (20% loading wet support, 0.2 g, 0.14 mmol) and the reaction was stirred at room temperature under an atmosphere of hydrogen for 16 hours. The reaction mixture was filtered through a pad of Celite® and the filtrate was concentrated under reduced pressure. The crude material was purified by column chromatography on silica gel, eluting with 50-60% ethyl acetate in hexane to give tert-butyl rel-(4aS,7R,7aS)-7-hydroxy-octahydrocyclopenta[b][1,4]oxazine-4-carboxylate (240 mg, 0.99 mmol, 89% yield) as a white semi-solid. 1H NMR (400 MHz, DMSO-d6) δ 5.02 (s, 1H), 3.89 (ddd, 1H), 3.85-3.74 (m, 1H), 3.67 (d, 1H), 3.45 (td, 1H), 3.02 (dd, 1H), 2.84-2.70 (m, 2H), 2.29-2.14 (m, 1H), 2.03-1.85 (m, 1H), 1.87-1.68 (m, 1H), 1.25-1.12 (m, 1H). LCMS (Method 4-Column 1): Retention Time=1.61 minutes, [MH]+=244.

### Synthesis of tert-butyl rel-(4aS,7S,7aS)-7-hydroxy-octahydrocyclopenta[b][1,4]oxazine-4-carboxylate

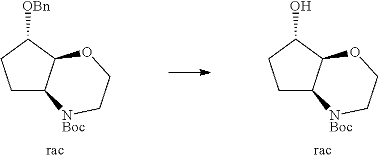 [SMILES: CC(C)(C)OC(=O)N1CCO[C@@H]2[C@@H](OCc3ccccc3)CC[C@@H]21 ; CC(C)(C)OC(=O)N1CCO[C@@H]2[C@@H](O)CC[C@@H]21]

To a solution of tert-butyl rel-(4aS,7S,7aS)-7-(benzyloxy)-octahydrocyclopenta[b][1,4]oxazine-4-carboxylate (1.0 g, 3.0 mmol) in methanol (30 mL) was added palladium hydroxide on carbon (200% loading wet support, 546 mg, 0.39 mmol) and the reaction mixture was stirred at room temperature under hydrogen atmosphere for 16 hours. The reaction mixture was filtered through a Celite® plug and the filtrate was concentrated under reduced pressure. The crude material was purified by column chromatography on silica gel eluting with 25-30% ethyl acetate in hexane gradient to give tert-butyl rel-(4aS,7S,7aS)-7-hydroxy-octahydrocyclopenta[b][1,4]oxazine-4-carboxylate (600 mg, 2.5 mmol, 82% yield) as an off white solid. 1H NMR (400 MHz, Chloroform-d) δ 4.54-4.24 (m, 1H), 4.20-4.03 (m, 1H), 3.92-3.54 (m, 3H), 3.52-3.37 (m, 1H), 3.16-2.90 (m, 1H), 2.35-2.19 (m, 1H), 2.05-1.86 (m, 1H), 1.79-1.68 (m, 1H), 1.59-1.51 (m, 1H), 1.47 (s, 9H). LCMS (Method 4-Column 1): Retention Time=1.65 minutes, [(M−56)H]+=188.

### Synthesis of [(2S,6S)-4-benzyl-6-ethylmorpholin-2-yl]methanol and [(2S,6R)-4-benzyl-6-ethylmorpholin-2-yl]methanol

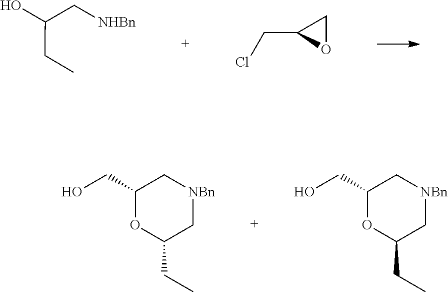

The reaction was performed in 3 parallel batches of 3.3 g of 1-(benzylamino)butan-2-ol, which were combined after work-up. To a solution of 1-(benzylamino)butan-2-ol (9.9 g, 55.2 mmol) in toluene (70 mL) was added dropwise (R)-(−)-epichlorohydrin (7.7 g, 82.8 mmol) followed by portion-wise addition of lithium perchlorate (5.9 g, 55.2 mmol). The reaction was stirred at room temperature for 16 hours. After 16 hours was added dropwise sodium methoxide (30% solution in methanol, 27.9 mL, 165.7 mmol) and the reaction mixture was stirred at room temperature for a further 16 hours. The reaction mixture was partitioned between water and ethyl acetate. The aqueous layer was further extracted with ethyl acetate and the combined organic layer was dried over anhydrous sodium sulfate and concentrated under reduced pressure. The crude material was purified by column chromatography on silica gel eluting with 0-20% ethyl acetate in hexane gradient to give [(2S,6S)-4-benzyl-6-ethylmorpholin-2-yl]methanol (3.6 g, 15.3 mmol, 28% yield) and [(2S,6R)-4-benzyl-6-ethylmorpholin-2-yl]methanol (3.0 g, 12.7 mmol, 23% yield)

### [(2S,6S)-4-benzyl-6-ethylmorpholin-2-yl]methanol

1H NMR (400 MHz, DMSO-d6) δ 7.36-7.19 (m, 5H), 4.61 (t, 1H), 3.54-3.47 (m, 1H), 3.47-3.36 (m, 3H), 3.29-3.19 (m, 2H), 2.79-2.67 (m, 2H), 1.73-1.61 (m, 2H), 1.46-1.26 (m, 2H), 0.84 (t, 3H). LCMS (Method 8-Column 1): Retention Time=4.26 minutes, [MH]+=236.

### [(2S,6R)-4-benzyl-6-ethylmorpholin-2-yl]methanol

1H NMR (400 MHz, DMSO-d6) δ 7.37-7.19 (m, 5H), 4.55 (t, 1H), 3.68-3.59 (m, 1H), 3.59-3.51 (m, 1H), 3.51-3.38 (m, 4H), 2.45-2.30 (m, 2H), 2.23-2.08 (m, 2H), 1.72-1.57 (m, 1H), 1.49-1.35 (m, 1H), 0.80 (t, 3H). LCMS (Method 8-Column 1): Retention Time=4.24 minutes, [MH]+=236.

### Synthesis of tert-butyl (2S,6S)-2-ethyl-6-(hydroxymethyl)morpholine-4-carboxylate

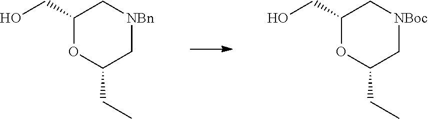 [SMILES: CC[C@H]1CN(Cc2ccccc2)C[C@@H](CO)O1 ; CC[C@H]1CN(C(=O)OC(C)(C)C)C[C@@H](CO)O1]

To a solution of [(2S,6S)-4-benzyl-6-ethylmorpholin-2-yl]methanol (3.4 g, 14.4 mmol) in methanol (68 mL) was added palladium hydroxide on carbon (20% loading wet support, 1.7 g, 1.2 mmol) and the reaction mixture was stirred at room temperature for 16 hours under an atmosphere of hydrogen. The reaction mixture was filtered through a Celite® plug and the filtrate was concentrated under reduced pressure to give intermediate [(2S,6S)-6-ethylmorpholin-2-yl]methanol as a pale yellow liquid.

To a solution of [(2S,6S)-6-ethylmorpholin-2-yl]methanol (2.0 g, 13.8 mmol) in dichloromethane (55 mL): water (18 mL) cooled to 0° C., was added sodium hydroxide (550 mg, 13.8 mmol) and the reaction mixture was stirred for 10 minutes before addition of di-tert-butyl dicarbonate (3.3 g, 15.2 mmol) and the reaction mixture was stirred at room temperature for 16 hours. The reaction was partitioned between water and dichloromethane and the aqueous layer was further extracted with dichloromethane. The combined organic layer was dried over anhydrous sodium sulfate and concentrated under reduced pressure. The crude material was purified by column chromatography on silica gel eluting with 0-30% ethyl acetate in hexane gradient to give tert-butyl (2S,6S)-2-ethyl-6-(hydroxymethyl)morpholine-4-carboxylate (2.4 g, 9.6 mmol, 68% yield) as a colourless liquid. 1H NMR (400 MHz, Chloroform-d) δ 4.14-3.75 (m, 2H), 3.75-3.62 (m, 1H), 3.62-3.47 (m, 2H), 3.44-3.23 (m, 1H), 2.79-2.36 (m, 2H), 2.02-1.88 (m, 1H), 1.58-1.48 (m, 2H), 1.46 (s, 9H), 0.96 (t, 3H). LCMS (Method 4-Column 7: Retention Time=1.84 minutes, [MH]+=246.

### Synthesis of tert-butyl (2R,6S)-2-ethyl-6-(hydroxymethyl)morpholine-4-carboxylate

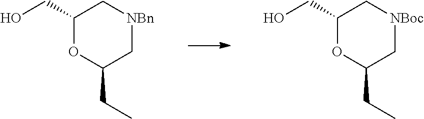 [SMILES: CC[C@@H]1CN(Cc2ccccc2)C[C@@H](CO)O1 ; CC[C@@H]1CN(C(=O)OC(C)(C)C)C[C@@H](CO)O1]

To a solution of [(2S,6R)-4-benzyl-6-ethylmorpholin-2-yl]methanol (2.8 g, 11.9 mmol) in methanol (56 mL) was added palladium hydroxide on carbon (20% loading wet support, 1.4 g, 1.0 mmol) and the reaction mixture was stirred at room temperature for 16 hours under an atmosphere of hydrogen. The reaction mixture was filtered through a Celite® plug and the filtrate was concentrated under reduced pressure to give intermediate [(2S,6R)-6-ethylmorpholin-2-yl]methanol.

To a solution of [(2S,6R)-6-ethylmorpholin-2-yl]methanol (1.6 g, 11.0 mmol) in dichloromethane/water (42:14 mL) cooled to 0° C., was added sodium hydroxide (440 mg, 11.0 mmol) and the reaction mixture was stirred for 10 minutes before addition of di-tert-butyl dicarbonate (2.6 g, 12.1 mmol) and the reaction mixture was stirred at room temperature for 16 hours. The reaction mixture was partitioned between water and dichloromethane. The aqueous layer was further extracted with dichloromethane and the combined organic layer was dried over anhydrous sodium sulfate and concentrated under reduced pressure. The crude material was purified by column chromatography on silica gel eluting with 0-30% ethyl acetate in hexane gradient to give tert-butyl (2R,6S)-2-ethyl-6-(hydroxymethyl)morpholine-4-carboxylate (1.8 g, 7.1 mmol, 62% yield) as a colourless liquid. 1H NMR (400 MHz, Chloroform-d) δ 3.89-3.77 (m, 1H), 3.77-3.13 (m, 7H), 2.09-1.79 (m, 1H), 1.71-1.61 (m, 1H), 1.55-1.48 (m, 1H), 1.46 (s, 8H), 0.94 (t, 3H). LCMS (Method 4-Column 7): Retention Time=1.75 minutes, [MH]+=246.

### Synthesis of rel-(2R)-2-[(2S,4R)-1-benzyl-4-(hydroxymethyl)azetidin-2-yl]-N-tert-butyl-2-hydroxyacetamide

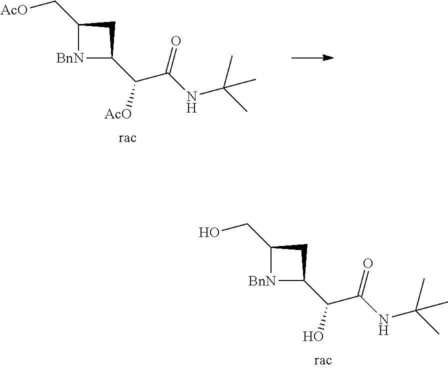 [SMILES: CC(=O)OC[C@H]1C[C@@H]([C@@H](OC(C)=O)C(=O)NC(C)(C)C)N1Cc1ccccc1 ; CC(C)(C)NC(=O)[C@H](O)[C@@H]1C[C@H](CO)N1Cc1ccccc1]

To a solution of [rel-(2R,4S)-4-[(1R)-1-acetyloxy-2-(tert-butylamino)-2-oxoethyl]-1-benzylazetidin-2-yl]methyl acetate (4.7 g, 12.0 mmol) in methanol (50 mL) cooled to 0° C., was added potassium hydroxide (2.0 g, 34.9 mmol). The reaction mixture was stirred for 1.5 hours at 0° C. The reaction mixture was quenched by addition of 10% aqueous ammonium chloride solution (100 mL) and the products were extracted with ethyl acetate. The combined organic layers were washed with water and dried over anhydrous sodium sulfate. The solvents were moved to give rel-(2R)-2-[(2S,4R)-1-benzyl-4-(hydroxymethyl)azetidin-2-yl]-N-tert-butyl-2-hydroxyacetamide (3.4 g, 11.1 mmol, 92% yield) as off white solid. The crude material was used directly for the next step. 1H NMR (400 MHz, Chloroform-d) δ 7.37-7.26 (m, 5H), 7.05 (s, 1H), 3.93 (td, 1H), 3.77 (d, 1H), 3.69 (d, 1H), 3.56 (d, 1H), 3.37-3.25 (m, 2H), 3.25-3.16 (m, 1H), 2.23-2.10 (m, 1H), 2.05-1.93 (m, 1H), 1.36 (s, 9H). LCMS (Method 5): Retention Time=1.47 minutes, [MH]+=307.

### Synthesis of rel-(1R,2S,5S)-6-benzyl-N-tert-butyl-3-oxa-6-azabicyclo[3.1.1]heptane-2-carboxamide

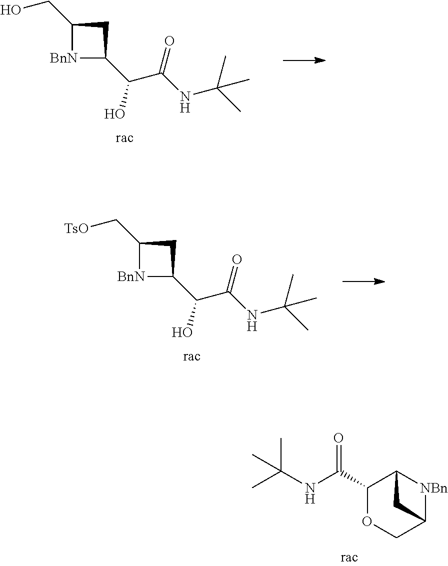 [SMILES: CC(C)(C)NC(=O)[C@H](O)[C@@H]1C[C@H](CO)N1Cc1ccccc1 ; Cc1ccc(S(=O)(=O)OC[C@H]2C[C@@H]([C@@H](O)C(=O)NC(C)(C)C)N2Cc2ccccc2)cc1 ; CC(C)(C)NC(=O)[C@H]1OC[C@@H]2C[C@H]1N2Cc1ccccc1]

To a solution of rel-(2R)-2-[(2S,4R)-1-benzyl-4-(hydroxymethyl)azetidin-2-yl]-N-tert-butyl-2-hydroxyacetamide (3.4 g, 11.1 mmol) in dichloromethane (75 mL) cooled to 0° C., was added 4-dimethylaminopyridine (136 mg, 1.1 mmol) and triethylamine (4.6 mL, 33.3 mmol). Then p-toluenesulfonyl chloride (2.1 g, 11.1 mmol) was added and the reaction mixture was warmed to room temperature and stirred overnight. The reaction mixture was partitioned between 10% aqueous sodium bicarbonate solution and dichloromethane. The aqueous layer was further extracted with dichloromethane and the combined organic layers were washed with water, dried over anhydrous sodium sulfate and concentrated under reduced pressure. The crude material was purified by flash column chromatography eluting with 25% ethyl acetate in petroleum ether to give intermediate [rel-(2R,4S)-1-benzyl-4-[(1R)-2-(tert-butylamino)-1-hydroxy-2-oxoethyl]azetidin-2-yl]methyl 4-methylbenzenesulfonate (3.0 g, 6.6 mmol, 59% yield) as off white solid. LCMS (Method 5): Retention Time=2.01 minutes, [MH]+=461.

To a stirred solution of [rel-(2R,4S)-1-benzyl-4-[(1R)-2-(tert-butylamino)-1-hydroxy-2-oxoethyl]azetidin-2-yl]methyl 4-methylbenzenesulfonate (3.0 g, 6.6 mmol) in N,N-dimethylformamide (20 mL) was added sodium hydride (57-63% oil dispersion, 342 mg, 8.6 mmol). The reaction mixture was stirred at room temperature overnight. The reaction mixture was quenched by addition of 10% aqueous ammonium chloride solution and the products were extracted with ethyl acetate. The combined organic layers were washed with water, dried over anhydrous sodium sulfate and concentrated under reduced pressure. The crude material was purified by flash column chromatography eluting with 60% ethyl acetate in petroleum ether to give rel-(1R,2S,5S)-6-benzyl-N-tert-butyl-3-oxa-6-azabicyclo[3.1.1]heptane-2-carboxamide (430 mg, 1.5 mmol, 23% yield) as brown sticky solid. 1H NMR (400 MHz, Chloroform-d) δ 7.35-7.27 (m, 4H), 7.24-7.17 (m, 1H), 6.78 (s, 1H), 4.30 (dt, 1H), 4.13 (d, 1H), 4.10-4.04 (m, 1H), 3.98-3.82 (m, 3H), 3.38 (t, 1H), 2.99-2.92 (m, 1H), 1.80 (d, 1H), 1.41 (s, 9H). LCMS (Method 7): Retention Time=2.06 minutes, [MH]+=289.

### Synthesis of tert-butyl rel-(1R,2S,5S)-2-(hydroxymethyl)-3-oxa-6-azabicyclo[3.1.1]heptane-6-carboxylate

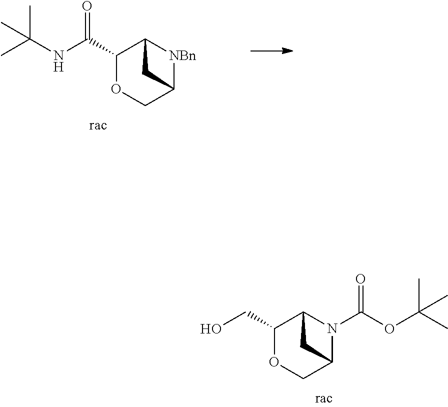 [SMILES: CC(C)(C)NC(=O)[C@H]1OC[C@@H]2C[C@H]1N2Cc1ccccc1 ; CC(C)(C)OC(=O)N1[C@@H]2CO[C@H](CO)[C@H]1C2]

A solution of rel-(1R,2S,5S)-6-benzyl-N-tert-butyl-3-oxa-6-azabicyclo[3.1.1]heptane-2-carboxamide (330 mg, 1.1 mmol) in concentrated hydrochloric acid (33 mL) was stirred at 55° C. for 10 hours. The solvents were removed under reduced pressure to give rel-(1R,2S,5S)-6-benzyl-3-oxa-6-azabicyclo[3.1.1]heptane-2-carboxylic acid, hydrochloride (460 mg, 1.71 mmol) as red hygroscopic solid which was used directly for the next step. LCMS (Method 7): Retention Time=1.42 minutes, [MH]+=233.8

To a solution of rel-(1R,2S,5S)-6-benzyl-3-oxa-6-azabicyclo[3.1.1]heptane-2-carboxylic acid, hydrochloride (430 mg, 1.6 mmol) in tetrahydrofuran (12 mL) cooled to 0° C., was added lithium aluminium hydride (2M in tetrahydrofuran, 3.2 mL, 6.4 mmol). The reaction mixture was stirred at 0-10° C. for 2 hours. The reaction mixture was quenched with 10% aqueous ammonium chloride solution and the products were extracted with ethyl acetate. The combined organic layers were washed water, dried over anhydrous sodium sulfate and the volatiles were removed under reduced pressure to give [rel-(1R,2S,5S)-6-benzyl-3-oxa-6-azabicyclo[3.1.1]heptan-2-yl]methanol (160 mg, 0.73 mmol, 46% yield) as reddish brown oil. LCMS (Method 7): Retention Time=1.10 minutes, [MH]+=220.

A solution of [rel-(1R,2S,5S)-6-benzyl-3-oxa-6-azabicyclo[3.1.1]heptan-2-yl]methanol (165 mg, 0.75 mmol) in methanol (15 mL) was degassed with nitrogen for 5 minutes before addition of palladium on carbon (10% w/w, 50 mg). The reaction mixture was placed under an atmosphere of hydrogen and it was stirred for 16 hours. The reaction mass was filtered through a pad of Celite® washing with methanol. The combined filtrate concentrated under reduced pressure to give [rel-(1R,2S,5S)-3-oxa-6-azabicyclo[3.1.1]heptan-2-yl]methanol (115 mg, 0.89 mmol) as a reddish brown oil. LCMS: Retention Time=0.37 minutes, [MH]+=130. The sample was analysed using the following conditions: Agilent 1290 Infinity 11 series, Column: ZORBAX XDB C-18 (50×4.6 mm) 3.5 μm, Column temperature: 25° C.; Mobile Phase A: 0.1% formic acid in water:acetonitrile (95:5); Mobile Phase B: acetonitrile; Flow rate: 1.5 mL\min, analysis time 5.5 min.

To a solution of rel-(1R,2S,5S)-3-oxa-6-azabicyclo[3.1.1]heptan-2-yl]methanol (110 mg, 0.85 mmol) in dichloromethane (15 mL) was added an aqueous solution of sodium hydroxide (34 mg, 0.85 mmol). The reaction mixture was cooled 0° C. and was added di-tert-butyl dicarbonate (204 mg, 0.94 mmol). The reaction mixture was stirred at room temperature for 5 hours. The reaction mixture was partitioned between water and dichloromethane, and the aqueous layer was further extracted with dichloromethane. The combined organic layer was washed with water and dried over anhydrous sodium sulfate. The crude material was purified by flash column chromatography on silica gel, eluting with 35% ethyl acetate in petroleum ether to give rel-(1R,2S,5S)-2-(hydroxymethyl)-3-oxa-6-azabicyclo[3.1.1]heptane-6-carboxylate (30 mg, 0.13 mmol, 15% yield) as brown oil. 1H NMR (400 MHz, Chloroform-d) δ 4.37-4.10 (m, 3H), 4.05-3.95 (m, 1H), 3.89-3.71 (m, 2H), 3.66-3.57 (m, 1H), 2.79-2.64 (m, 1H), 1.77 (d, 1H), 1.47 (s, 9H).

### Synthesis of 7-chloro-4-ethyl-5-methoxy-1,6-naphthyridine

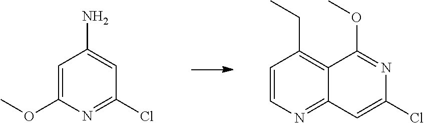

To a solution of 2-chloro-6-methoxypyridin-4-amine (5.1 g, 32.0 mmol) in 1,4-dioxane (100 mL) was added sulfuric acid (3.5 mL, 65.8 mmol) and the reaction mixture was heated to 110° C. before addition of pent-1-en-3-one (4.2 g, 49.3 mmol). The reaction mixture was heated at 110° C. for 5 hours. The reaction mixture was cooled to room temperature and poured into a chilled 10% aqueous sodium bicarbonate solution. The product was extracted with ethyl acetate. The combined organic layer was washed with water, brine, dried over anhydrous sodium sulphate and concentrated under reduced pressure. The crude material was purified by column chromatography on silica gel eluting with a 10-15% ethyl acetate in petroleum ether gradient to give 7-chloro-4-ethyl-5-methoxy-1,6-naphthyridine (1.7 g, 7.6 mmol, 24% yield) as yellow semi-solid. 1H NMR (400 MHz, Chloroform-d): δ 8.83 (d, 1H), 7.54 (s, 1H), 7.26 (d, 1H), 4.17 (s, 3H), 3.31-3.25 (m, 2H), 1.35-1.31 (m, 3H). UPLC: Retention Time=1.79 minutes, [MH]+=222.9. The sample was analysed using the following conditions: Shimadzu LCMS-2020; Column: Acquity UPLC BEH C18 1.7 μm, 50×2.1 mm ID: Column temperature: 25° C.; Mobile Phase A: 0.1% formic acid in water, Mobile Phase B: acetonitrile; Mobile phase gradient details: T=0 minutes (95% A, 5% B); T=2.5 minutes (0% A, 100% B); gradient to T=3 minutes (0% A, 100% B); end of run at T=4.0 minutes (95% A, 5% B). Flow rate: 0.8 mL\min, analysis time 4 minutes.

### Synthesis of 7-chloro-4-ethyl-1,6-naphthyridin-5-ol

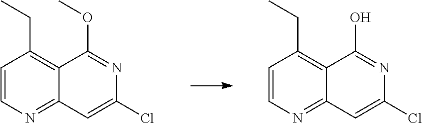

To a solution of 7-chloro-4-ethyl-5-methoxy-1,6-naphthyridine (1.1 g, 4.9 mmol) in dry acetic acid (10 mL) was added concentrated hydrochloric acid (10 mL, 408.7 mmol). The reaction mixture was heated to 140° C. for 15 minutes in the microwave. The cooled reaction mixture was neutralised by slowly pouring into a 10% aqueous sodium bicarbonate solution. The products were extracted with ethyl acetate and the combined organic layers were washed with water, brine, dried over anhydrous sodium sulfate and concentrated to give 7-chloro-4-ethyl-1,6-naphthyridin-5-ol (976 mg, 4.7 mmol, 95% yield) as red solid. No purification was carried out. 1H NMR (400 MHz, DMSO-d6): δ 12.43 (br s, 1H), 8.71 (d, 1H), 7.29 (d, 1H), 6.69 (s, 1H), 3.29-3.23 (m, 2H), 1.22-1.18 (m, 3H). UPLC: Retention Time=1.06 minutes, [MH]+=208.9. The sample was analysed using the following conditions: Shimadzu LCMS-2020; Column: Acquity UPLC BEH C18 1.7 μm, 50×2.1 mm ID; Column temperature: 25° C.; Mobile Phase A: 0.1% formic acid in water, Mobile Phase B: acetonitrile: Mobile phase gradient details: T=0 minutes (95% A, 5% B); T=2.5 minutes (0% A, 100% B); gradient to T=3 minutes (0% A, 100% B); end of run at T=4.0 minutes (95% A, 5% B). Flow rate: 0.8 mL\min, analysis time 4 minutes.

### Synthesis of 5,7-dichloro-4-ethyl-1,6-napthyridine

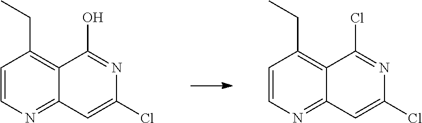

To a solution of 7-chloro-4-ethyl-1,6-naphthyridin-5-ol (1.4 g, 6.7 mmol) in phosphorous oxychloride (10.0 mL, 106.6 mmol) was added tetramethylammonium chloride (880 mg, 8.1 mmol). The reaction mixture was heated to 100° C. for 16 hours. The cooled reaction mixture was quenched by slow addition into a 10% aqueous sodium bicarbonate solution. The products were extracted in ethyl acetate and the combined organic layers were washed with water, brine, dried over anhydrous sodium sulfate and concentrated. The crude material was purified by column chromatography on silica gel eluting with 15% ethyl acetate in petroleum ether to give 5,7-dichloro-4-ethyl-1,6-naphthyridine (610 mg, 2.7 mmol, 40% yield) as light brown solid. 1H NMR (400 MHz, Chloroform-d): δ 8.95 (d, 1H), 795 (s, 1H), 7.42 (d, 1H), 3.56-3.51 (m, 2H), 1.45-1.41 (m, 3H). LCMS (Method 6): Retention Time=2.19 minutes, [MH]+=227. The sample was analysed using the following conditions: Agilent 1290 Infinity II series, Column: X-Bridge C8 (50×4.6 mm), 3.5 μm. Column temperature: 25° C. Mobile Phase A: H₂O+0.10% TFA, Mobile Phase B: MeCN. Mobile phase gradient details: T=0 minutes (95% A, 5% B); T=2.5 minutes (5% A, 95% B); gradient to T=4 minutes (5% A, 95% B); end of run at T=4.5 minutes (95% A, 5% B). Flow rate: 1.5 mL/min, analysis time 6.0 min. UV detection: maximum absorption.

### Synthesis of 5,7-dichloro-1,6-naphthyridin-4-ol

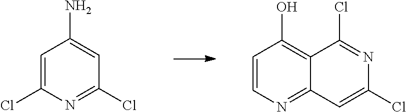 [SMILES: Nc1cc(Cl)nc(Cl)c1 ; Oc1ccnc2cc(Cl)nc(Cl)c12]

To a solution of 2,6-dichloropyridin-4-amine (10.0 g, 61.4 mmol) in 2-propanol (60 mL) was added 5-(methoxymethylidene)-2,2-dimethyl-1,3-dioxane-4,6-dione (12.0 g, 64.4 mmol) and the reaction mixture was heated to 100° C. for 1 hour. The reaction mixture was cooled to room temperature and the white precipitate was collected by filtration, washed with 2-propanol and dried under reduced pressure.

The crude intermediate was dissolved in 1,2-dichlorobenzene (48.0 mL, 424.5 mmol) in 100 mL sealed tube and the reaction mixture was stirred at 250° C. for 30 minutes. The reaction mixture was cooled to room temperature and concentrated under reduced pressure. The crude material was trituration with diethyl ether (3×70 mL) followed by ethyl acetate (3.50 mL) to give 5,7-dichloro-1,6-naphthyridin-4-ol (5.5 g, 23.5 mmol, 38% yield) as beige coloured solid. 1H NMR (400 MHz, DMSO-d6) δ 12.10 (s, 1H), 7.92 (app t, 1H), 7.46 (s, 1H), 6.16 (d, 1H). LC MS (Method 4-Column 7): Retention Time: 1.25 minutes, [MH]+=2.15.

### Synthesis of 5,7-dichloro-4-methoxy-1,6-naphthyridine

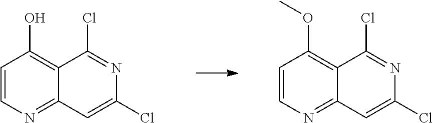

To a solution of 5,7-dichloro-1,6-naphthyridin-4-ol (2.6 g, 12.1 mmol) in toluene (80 mL) in a 100 mL sealed tube, were added silver carbonate (6.7 g, 24.2 mmol) and iodomethane (6.7 mL, 107.6 mmol) and the reaction mixture was heated to 100° C. for 2 hours. The cooled reaction mixture was concentrated under reduced pressure and purified by column chromatography on silica gel eluting with 2-5% methanol in dichloromethane gradient to give 5,7-dichloro-4-methoxy-1,6-naphthyridine (790 mg, 3.43 mmol, 28% yield). 1H NMR (400 MHz, DMSO-d6) δ 8.96 (d, 1H), 7.97 (s, 1H), 7.28 (d, 1H), 4.08 (s, 3H). LCMS (Method 4-Column 7): Retention Time=1.82 minutes, [MH]+=229.1.

### Synthesis of [(2S,6S)-4-benzyl-6-(propan-2-yl)morpholin-2-yl]methanol and [(2S,6R)-4-benzyl-6-(propan-2-yl)morpholin-2-yl]methanol

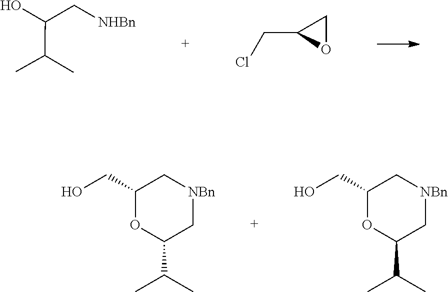

To a solution of 1-(benzylamino)-3-methylbutan-2-ol (1.3 g, 6.63 mmol) in toluene (13 mL) was added dropwise (R)-(−)-epichlorohydrin (920 mg, 9.94 mmol) followed by portionwise addition of lithium perchlorate (700 mg, 6.63 mmol). The reaction was stirred at room temperature for 16 hours. After 16 hours was added dropwise sodium methoxide (30% solution in methanol, 3.8 mL, 19.9 mmol) and the reaction mixture was stirred at room temperature for a further 48 hours. The reaction mixture was partitioned between water and ethyl acetate. The aqueous layer was further extracted with ethyl acetate and the combined organic layer was dried over anhydrous sodium sulfate and concentrated under reduced pressure. The crude material was purified by column chromatography on silica gel eluting with 0-20% ethyl acetate in hexane gradient to give [(2S,6S)-4-benzyl-6-(propan-2-yl)morpholin-2-yl]methanol (470 mg, 1.66 mmol, 25% yield) and [(2S,6R)-4-benzyl-6-(propan-2-yl)morpholin-2-yl]methanol (500 mg, 2.01 mmol, 30% yield).

### [(2S,6S)-4-benzyl-6-(propan-2-yl)morpholin-2-yl]methanol

1H NMR (400 MHz, DMSO-d6) δ 7.47-7.12 (m, 5H), 4.63-4.47 (m, 1H), 3.70-3.58 (m, 1H), 3.54-3.40 (m, 3H), 3.40-3.35 (m, 1H), 3.25-3.15 (m, 1H), 2.46-2.38 (m, 1H), 2.35-2.13 (m, 3H), 2.08-1.94 (m, 1H), 0.87 (d, 3H), 0.72 (d, 3H). LCMS (Method 8-Column 1): Retention Time=4.79 minutes, [MH]+=250.1.

### [(2S,6R)-4-benzyl-6-(propan-2-yl)morpholin-2-yl]methanol

1H NMR (400 MHz, DMSO-d6) δ 7.38-7.20 (m, 5H), 4.61-4.52 (m, 1H), 3.54 (d, 1H), 3.47-3.35 (m, 3H), 3.28-3.20 (m, 1H), 3.19-3.10 (m, 1H), 2.86-2.68 (m, 2H), 1.82-1.68 (m, 1H), 1.68-1.50 (m, 2H), 0.88 (d, 3H), 0.81 (d, 3H). LCMS (Method 8-Column 1): Retention Time=4.79 minutes, [MH]+=250.1.

### Synthesis of tert-butyl (2S,6S)-2-(hydroxymethyl)-6-(propan-2-yl)morpholine-4-carboxylate

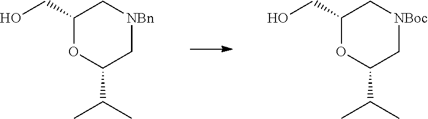 [SMILES: CC(C)[C@H]1CN(Cc2ccccc2)C[C@@H](CO)O1 ; CC(C)[C@H]1CN(C(=O)OC(C)(C)C)C[C@@H](CO)O1]

To a solution of [(2S,6S)-4-benzyl-6-(propan-2-yl)morpholin-2-yl]methanol (430 mg, 1.72 mmol) in methanol (12 mL) was added palladium hydroxide on carbon (220% loading wet support, 206 mg, 0.15 mmol) and the reaction mixture was stirred at room temperature for 16 hours under an atmosphere of hydrogen. The reaction mixture was filtered through a Celite® plug and the filtrate was concentrated under reduced pressure to give intermediate [(2S,6S)-6-(propan-2-yl)morpholin-2-yl]methanol as a colourless oil.

To a solution of [(2S,6S)-6-(propan-2-yl)morpholin-2-yl]methanol (230 mg, 1.44 mmol) in dichloromethane (9 mL): water (3 mL) cooled to 0° C., was added sodium hydroxide (58 mg, 1.44 mmol) and the reaction mixture was stirred for 10 minutes before addition of di-tert-butyl dicarbonate (347 mg, 1.59 mmol) and the reaction mixture was stirred at room temperature for 16 hours. The reaction was partitioned between water and dichloromethane and the aqueous layer was further extracted with dichloromethane. The combined organic layer was dried over anhydrous sodium sulfate and concentrated under reduced pressure. The crude material was purified by column chromatography on silica gel eluting with 10-20% ethyl acetate in hexane gradient to give tert-butyl (2S,6S)-2-(hydroxymethyl)-6-(propan-2-yl)morpholine-4-carboxylate (240 mg, 0.93 mmol, 54% yield) as a colourless oil. 1H NMR (400 MHz, Chloroform-d) δ 3.86-3.76 (m, 1H), 3.72-3.48 (m, 3H), 3.46-3.11 (m, 3H), 1.99-1.69 (m, 3H), 1.46 (s, 9H), 0.97 (d, 3H), 0.93 (d, 3H). LCMS (Method 4-Column 7: Retention Time=1.94 minutes, [MH]+=260.

### Synthesis of tert-butyl (2S,6R)-2-(hydroxymethyl)-6-(propan-2-yl)morpholine-4-carboxylate

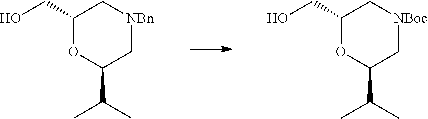 [SMILES: CC(C)[C@@H]1CN(Cc2ccccc2)C[C@@H](CO)O1 ; CC(C)[C@@H]1CN(C(=O)OC(C)(C)C)C[C@@H](CO)O1]

To a solution of [(2S,6R)-4-benzyl-6-(propan-2-yl)morpholin-2-yl]methanol (460 mg, 1.84 mmol) in methanol (12 mL) was added palladium hydroxide on carbon (20% loading wet support, 220 mg, 0.16 mmol) and the reaction mixture was stirred at room temperature for 16 hours under an atmosphere of hydrogen. The reaction mixture was filtered through a Celite® plug and the filtrate was concentrated under reduced pressure to give intermediate [(2S,6R)-6-(propan-2-yl)morpholin-2-yl]methanol.

To a solution of [(2S,6R)-6-(propan-2-yl)morpholin-2-yl]methanol (260 mg, 1.63 mmol) in dichloromethane/water (9:3 mL) cooled to 0° C., was added sodium hydroxide (65 mg, 1.63 mmol) and the reaction mixture was stirred for 10 minutes before addition of di-tert-butyl dicarbonate (392 mg, 1.80 mmol) and the reaction mixture was stirred at room temperature for 16 hours. The reaction mixture was partitioned between water and dichloromethane. The aqueous layer was further extracted with dichloromethane and the combined organic layer was dried over anhydrous sodium sulfate and concentrated under reduced pressure. The crude material was purified by column chromatography on silica gel eluting with 10-20% ethyl acetate in hexane gradient to give tert-butyl (2S,6R)-2-(hydroxymethyl)-6-(propan-2-yl)morpholine-4-carboxylate (310 mg, 1.20 mmol, 65% yield) as a colourless oil. 1H NMR (400 MHz, Chloroform-d) δ 4.15-3.73 (m, 2H), 3.68 (dd, 1H), 3.61-3.45 (m, 2H), 3.18-3.06 (m, 1H), 2.72-2.42 (m, 2H), 1.78-1.66 (m, 1H), 1.46 (s, 9H), 0.98 (d, 3H), 0.93 (d, 3H). LCMS (Method 4-Column 7: Retention Time=2.06 minutes, [MH]+=260.

### Synthesis of [(2S,6S)-4-benzyl-6-tert-butylmorpholin-2-yl]methanol and [(2S,6R)-4-benzyl-6-tert-butylmorpholin-2-yl]methanol

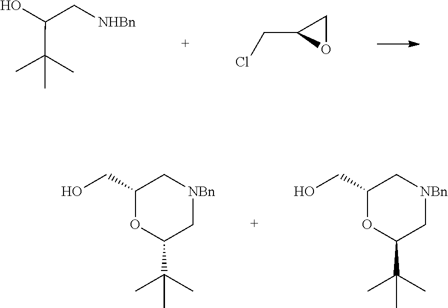

To a solution of 1-(benzylamino)-3,3-dimethylbutan-2-ol (950 mg, 4.58 mmol) in toluene (4 mL) was added dropwise (R)-(−)-epichlorohydrin (636 mg, 6.87 mmol) followed by portionwise addition of lithium perchlorate (488 mg, 4.58 mmol). The reaction was stirred at room temperature for 16 hours. After 16 hours was added dropwise sodium methoxide (30% solution in methanol, 2.6 mL, 13.7 mmol) and the reaction mixture was stirred at room temperature for 13 days. The reaction mixture was partitioned between water and ethyl acetate. The aqueous layer was further extracted with ethyl acetate and the combined organic layer was dried over anhydrous sodium sulfate and concentrated under reduced pressure. The crude material was purified by column chromatography on silica gel eluting with 10-20% ethyl acetate in hexane gradient to give [(2S,6S)-4-benzyl-6-tert-butylmorpholin-2-yl]methanol (140 mg, 0.47 mmol, 10% yield) and [(2S,6R)-4-benzyl-6-tert-butylmorpholin-2-yl]methanol (180 mg, 0.68 mmol, 15% yield).

### [(2S,6S)-4-benzyl-6-tert-butylmorpholin-2-yl]methanol

1H NMR (400 MHz, DMSO-d6) δ 7.36-7.20 (m, 5H), 4.52-4.40 (m, 1H), 3.78-3.70 (m, 1H), 3.69-3.58 (m, 2H), 3.49 (d, 1H), 3.38 (d, 1H), 3.31-3.23 (m, 1H), 2.76-2.63 (m, 2H), 2.06-1.91 (m, 1H), 1.80 (t, 1H), 0.82 (s, 9H). LCMS (Method 8-Column 1): Retention Time=5.77 minutes, [MH]+=264

### [(2S,6R)-4-benzyl-6-tert-butylmorpholin-2-yl]methanol

1H NMR (400 MHz, DMSO-d6) δ 7.36-7.19 (m, 5H), 4.59-4.50 (m, 1H), 3.56 (d, 1H), 3.45-3.34 (m, 3H), 3.28-3.18 (m, 1H), 3.11 (dd, 1H), 2.80-2.70 (m, 2H), 1.80 (t, 1H), 1.60 (t, 1H), 0.85 (s, 9H). LCMS (Method 8-Column 1): Retention Time=5.66 minutes, [MH]+=264.

### Synthesis of tert-butyl (2S,6S)-2-tert-butyl-6-(hydroxymethyl)morpholine-4-carboxylate

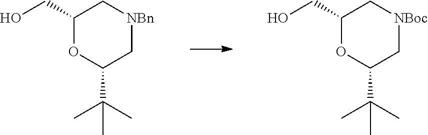 [SMILES: CC(C)(C)[C@H]1CN(Cc2ccccc2)C[C@@H](CO)O1 ; CC(C)(C)OC(=O)N1C[C@@H](CO)O[C@@H](C(C)(C)C)C1]

To a solution of [(2S,6S)-4-benzyl-6-tert-butylmorpholin-2-yl]methanol (200 mg, 0.76 mmol) in methanol (10 mL) was added palladium hydroxide on carbon (20% loading wet support, 96 mg, 0.07 mmol) and the reaction mixture was stirred at room temperature for 16 hours under an atmosphere of hydrogen. The reaction mixture was filtered through a Celite® plug and the filtrate was concentrated under reduced pressure to give intermediate [(2S,6S)-6-tert-butylmorpholin-2-yl]methanol as a pale yellow oil.

To a solution of [(2S,6S)-6-tert-butylmorpholin-2-yl]methanol (120 mg, 0.69 mmol) in dichloromethane (7 mL): water (2 mL) cooled to 0° C., was added sodium hydroxide (28 mg, 0.69 mmol) and the reaction mixture was stirred for 10 minutes before addition of di-tert-butyl dicarbonate (166 mg, 0.76 mmol) and the reaction mixture was stirred at room temperature for 16 hours. The reaction was partitioned between water and dichloromethane and the aqueous layer was further extracted with dichloromethane. The combined organic layer was dried over anhydrous sodium sulfate and concentrated under reduced pressure. The crude material was purified by column chromatography on silica gel eluting with 10-20% ethyl acetate in hexane gradient to give tert-butyl (2S,6S)-2-tert-butyl-6-(hydroxymethyl)morpholine-4-carboxylate (100 mg, 0.37 mmol, 48% yield) as a pale yellow oil. 1H NMR (400 MHz, DMSO-d6) δ 4.60 (t, 1H), 3.88-3.63 (m, 3H), 3.54-3.39 (m. 2H), 3.18 (dd, 1H), 3.06-2.94 (m, 1H), 1.39 (s, 9H), 0.85 (s, 9H). LCMS (Method 4-Column 7: Retention Time=2.18 minutes, [MH]+=274.

### Synthesis of tert-butyl (2R,6S)-2-tert-butyl-6-(hydroxymethyl)morpholine-4-carboxylate

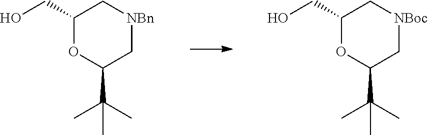 [SMILES: CC(C)(C)[C@@H]1CN(Cc2ccccc2)C[C@@H](CO)O1 ; CC(C)(C)OC(=O)N1C[C@@H](CO)O[C@H](C(C)(C)C)C1]

To a solution of [(2S,6R)-4-benzyl-6-tert-butylmorpholin-2-yl]methanol (260 mg, 0.99 mmol) in methanol (13 mL) was added palladium hydroxide on carbon (20% loading wet support, 125 mg, 0.09 mmol) and the reaction mixture was stirred at room temperature for 16 hours under an atmosphere of hydrogen. The reaction mixture was filtered through a Celite® plug and the filtrate was concentrated under reduced pressure to give intermediate [(2S,6R)-6-tert-butylmorpholin-2-yl]methanol.

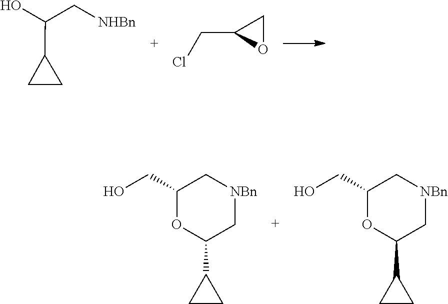

To a solution of 2-(benzylamino)-1-cyclopropylethanol (500 mg, 2.61 mmol) in toluene (7 mL) was added dropwise (R)-(−)-epichlorohydrin (363 mg, 3.92 mmol) followed by portionwise addition of lithium perchlorate (278 mg, 2.61 mmol). The reaction was stirred at room temperature for 16 hours. After 16 hours was added dropwise sodium methoxide (30% solution in methanol, 1.5 mL, 7.84 mmol) and the reaction mixture was stirred at room temperature for 16 hours. The reaction mixture was partitioned between water and ethyl acetate. The aqueous layer was further extracted with ethyl acetate and the combined organic layer was dried over anhydrous sodium sulfate and concentrated under reduced pressure. The crude material was purified by column chromatography on silica gel eluting with 10-20% ethyl acetate in hexane gradient to give [(2S,6S)-4-benzyl-6-cyclopropylmorpholin-2-yl]methanol (220 mg, 0.82 mmol, 31% yield) and [(2S,6R)-4-benzyl-6-cyclopropylmorpholin-2-yl]methanol (270 mg, 1.09 mmol, 42% yield).

### [(2S,6S)-4-benzyl-6-cyclopropylmorpholin-2-yl]methanol

1H NMR (400 MHz, DMSO-d6) δ 7.45-7.14 (m, 5H), 4.56 (t, 1H), 3.83-3.71 (m, 1H), 3.51-3.37 (m, 3H), 2.94-2.82 (m, 1H), 2.42-2.30 (m, 2H), 2.15 (dd, 1H), 1.36-1.17 (m, 2H), 0.92-0.72 (m, 1H), 0.50-0.34 (m, 2H), 0.30-0.16 (m, 1H), 0.16-0.03 (m, 1H). LCMS (Method 8-Column 1): Retention Time=4.39 minutes, [MH]+=248

### [(2S,6R)-4-benzyl-6-cyclopropylmorpholin-2-yl]methanol

1H NMR (400 MHz, DMSO-d6) S 737-7.18 (m, 5H), 4.63 (t, 1H), 3.51 (d, 1H), 3.45-3.36 (m, 3H), 3.29-3.18 (m, 1H), 2.81-2.70 (m, 2H), 1.85 (t, 1H), 1.67 (t, 1H), 0.90-0.80 (m, 1H), 0.80-0.70 (m, 1H), 0.46-0.31 (m, 2H), 0.31-0.19 (m, 1H), 0.19-0.09 (m, 1H). LCMS (Method 8-Column 1): Retention Time=4.35 minutes, [MH]+=248.

### Synthesis of tert-butyl (2S,6S)-2-cyclopropyl-6-(hydroxymethyl)morpholine-4-carboxylate

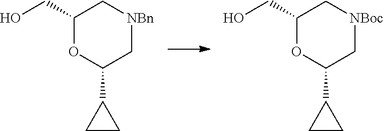 [SMILES: OC[C@@H]1CN(Cc2ccccc2)C[C@H](C2CC2)O1 ; CC(C)(C)OC(=O)N1C[C@@H](CO)O[C@@H](C2CC2)C1]

To a solution of [(2S,6S)-4-benzyl-6-cyclopropylmorpholin-2-yl]methanol (220 mg, 0.89 mmol) in methanol (10 mL) was added palladium hydroxide on carbon (20% loading wet support, 112 mg, 0.08 mmol) and the reaction mixture was stirred at room temperature for 16 hours under an atmosphere of hydrogen. The reaction mixture was filtered through a Celite® plug and the filtrate was concentrated under reduced pressure to give intermediate [(2S,6S)-6-cyclopropylmorpholin-2-yl]methanol as a pale yellow oil.

To a solution of [(2S,6S)-6-cyclopropylmorpholin-2-yl]methanol (130 mg, 0.83 mmol) in dichloromethane (6 mL): water (2 mL) cooled to 0° C., was added sodium hydroxide (33 mg, 0.83 mmol) and the reaction mixture was stirred for 10 minutes before addition of di-tert-butyl dicarbonate (198 mg, 0.91 mmol) and the reaction mixture was stirred at room temperature for 16 hours. The reaction was partitioned between water and dichloromethane and the aqueous layer was further extracted with dichloromethane. The combined organic layer was dried over anhydrous sodium sulfate and concentrated under reduced pressure. The crude material was purified by column chromatography on silica gel eluting with 10-20% ethyl acetate in hexane gradient to give tert-butyl (2S,6S)-2-cyclopropyl-6-(hydroxymethyl)morpholine-4-carboxylate (140 mg, 0.54 mmol, 61% yield) as a pale yellow oil. 1H NMR (400 MHz, DMSO-d6) δ 4.69 (t, 1H), 3.79-3.68 (m, 1H), 3.64-3.37 (m, 4H), 3.21-2.78 (m, 3H), 1.40 (s, 9H), 1.07-0.89 (m, 1H), 0.54-0.38 (m, 2H), 0.37-0.15 (m, 2H). LCMS (Method 4-Column 7: Retention Time=1.79 minutes, [MH]+=258.

### Synthesis of tert-butyl (2R,6S)-2-cyclopropyl-6-(hydroxymethyl)morpholine-4-carboxylate

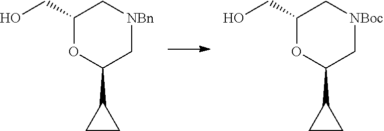 [SMILES: OC[C@@H]1CN(Cc2ccccc2)C[C@@H](C2CC2)O1 ; CC(C)(C)OC(=O)N1C[C@@H](CO)O[C@H](C2CC2)C1]

To a solution of [(2S,6R)-4-benzyl-6-cyclopropylmorpholin-2-yl]methanol (270 mg, 1.09 mmol) in methanol (10 mL) was added palladium hydroxide on carbon (20% loading wet support, 138 mg, 0.10 mmol) and the reaction mixture was stirred at room temperature for 16 hours under an atmosphere of hydrogen. The reaction mixture was filtered through a Celite® plug and the filtrate was concentrated under reduced pressure to give [(2S,6R)-6-cyclopropylmorpholin-2-yl]methanol.

To a solution of [(2S,6R)-6-cyclopropylmorpholin-2-yl]methanol (150 mg, 0.95 mmol) in dichloromethane/water (8:3 mL) cooled to 0° C., was added sodium hydroxide (38 mg, 0.95 mmol) and the reaction mixture was stirred for 10 minutes before addition of di-tert-butyl dicarbonate (229 mg, 1.05 mmol) and the reaction mixture was stirred at room temperature for 16 hours. The reaction mixture was partitioned between water and dichloromethane. The aqueous layer was further extracted with dichloromethane and the combined organic layer was dried over anhydrous sodium sulfate and concentrated under reduced pressure. The crude material was purified by column chromatography on silica gel eluting with 10-20% ethyl acetate in hexane gradient to give tert-butyl (2R,6S)-2-cyclopropyl-6-(hydroxymethyl)morpholine-4-carboxylate (110 mg, 0.43 mmol, 39% yield) as a pale yellow oil. 1H NMR (400 MHz, DMSO-d6) δ 4.77 (t, 1H), 3.95-3.71 (m, 2H), 3.50-3.39 (m, 1H), 3.32-3.19 (m, 2H), 2.75-2.54 (m, 3H), 1.40 (s, 9H), 0.90-0.68 (m, 1H), 0.51-0.39 (m, 2H), 0.37-0.27 (m, 1H), 0.27-0.16 (m, 1H). LCMS (Method 4-Column 7): Retention Time=1.84 minutes, [MH]+=285.

### Synthesis of [(2S,6R)-4-benzyl-6-(difluoromethyl)morpholin-2-yl]methanol and [(2S,6S)-4-benzyl-6-(difluoromethyl)morpholin-2-yl]methanol

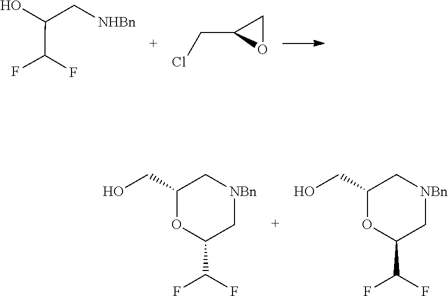

To a solution of 3-(benzylamino)-1,1-difluoropropan-2-ol (1.2 g, 5.96 mmol) in toluene (12 mL) was added dropwise (R)-(−)-epichlorohydrin (552 mg, 5.96 mmol) followed by portionwise addition of lithium perchlorate (634 mg, 5.96 mmol). The reaction was stirred at room temperature for 16 hours. After 16 hours was added dropwise sodium methoxide (30% solution in methanol, 3.4 mL, 17.9 mmol) and the reaction mixture was stirred at room temperature for 16 hours. The reaction mixture was partitioned between water and ethyl acetate. The aqueous layer was further extracted with ethyl acetate and the combined organic layer was dried over anhydrous sodium sulfate and concentrated under reduced pressure. The crude material was purified by column chromatography on silica gel eluting with 10-15% ethyl acetate in hexane gradient to give [(2S,6R)-4-benzyl-6-(difluoromethyl)morpholin-2-yl]methanol (220 mg, 0.82 mmol, 31% yield) and [(2S,6S)-4-benzyl-6-(difluoromethyl)morpholin-2-yl]methanol (270 mg, 1.09 mmol, 42% d yield).

### [(2S,6R)-4-benzyl-6-(difluoromethyl)morpholin-2-yl]methanol

1H NMR (400 MHz, DMSO-d6) δ 7.38-7.20 (m, 5H), 6.55-6.16 (m, 1H), 4.71 (t, 1H), 3.91-3.74 (m, 2H), 3.54-3.43 (m, 3H), 3.43-3.36 (m, 1H), 2.72-2.54 (m, 2H), 2.41-2.28 (, 1H), 2.14 (dd, H). LCMS (Method 8-Column 1): Retention Time=5.05 minutes, [MH]+=258.

### [(2S,6S)-4-benzyl-6-(difluoromethyl)morpholin-2-yl]methanol

1H NMR (400 MHz, DMSO-d6) δ 7.43-7.17 (m, 5H), 5.99 (td, 1H), 4.75 (t, 1H), 3.86-3.72 (m, 1H), 3.58-3.49 (m, 2H), 3.47-3.38 (m, 1H), 3.32-3.17 (m, 2H), 2.83-2.71 (m, 2H), 1.92 (t, 1H), 1.79 (t, 1H). LCMS (Method 8-Column 1): Retention Time=4.21 minutes, [MH]+=258.

### Synthesis of tert-butyl (2R,6S)-2-(difluoromethyl)-6-(hydroxymethyl)morpholine-4-carboxylate

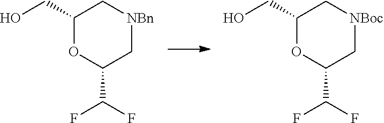 [SMILES: OC[C@@H]1CN(Cc2ccccc2)C[C@H](C(F)F)O1 ; CC(C)(C)OC(=O)N1C[C@@H](CO)O[C@@H](C(F)F)C1]

To a solution of [(2S,6R)-4-benzyl-6-(difluoromethyl)morpholin-2-yl]methanol (600 mg, 2.33 mmol) in methanol (6 mL) was added palladium hydroxide on carbon (20% loading wet support, 1.3 g, 0.93 mmol) and the reaction mixture was stirred at room temperature for 16 hours under an atmosphere of hydrogen. The reaction mixture was filtered through a Celite® plug and the filtrate was concentrated under reduced pressure to give intermediate [(2S,6R)-6-(difluoromethyl)morpholin-2-yl]methanol as a pale yellow oil.

To a solution of [(2S,6R)-6-(difluoromethyl)morpholin-2-yl]methanol (350 mg, 2.09 mmol) in dichloromethane (8 mL) cooled to 0° C., was added an aqueous solution of sodium hydroxide (1 M, 2.1 mL, 2.09 mmol) and the reaction mixture was stirred for 10 minutes before addition of di-tert-butyl dicarbonate (503 mg, 2.30 mmol) and the reaction mixture was stirred at room temperature for 16 hours. The reaction was partitioned between water and dichloromethane and the aqueous layer was further extracted with dichloromethane. The combined organic layer was dried over anhydrous sodium sulfate and concentrated under reduced pressure. The crude material was purified by column chromatography on silica gel eluting with 25% ethyl acetate in hexane to give tert-butyl (2R,6S)-2-(difluoromethyl)-6-(hydroxymethyl)morpholine-4-carboxylate (350 mg, 1.18 mmol, 51% yield) as a pale yellow oil. 1H NMR (400 MHz, DMSO-d6) δ 6.18 (t, 1H), 4.83 (t, 1H), 4.01-3.83 (m, 1H), 3.82-3.72 (m, 1H), 3.72-3.36 (m, 5H), 3.27-2.88 (m, 1H), 1.40 (s, 9H). LCMS (Method 8-Column 1): Retention Time=7.08 minutes, [(M−Boc)H]+=168.

### Synthesis of tert-butyl (2S,6S)-2-(difluoromethyl)-6-(hydroxymethyl)morpholine-4-carboxylate

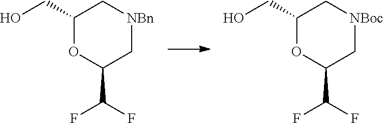 [SMILES: OC[C@@H]1CN(Cc2ccccc2)C[C@@H](C(F)F)O1 ; CC(C)(C)OC(=O)N1C[C@@H](CO)O[C@H](C(F)F)C1]

To a solution of [(2S,6S)-4-benzyl-6-(difluoromethyl)morpholin-2-yl]methanol (600 mg, 2.33 mmol) in methanol (6 mL) was added palladium hydroxide on carbon (20% loading wet support, 1.3 g, 0.93 mmol) and the reaction mixture was stirred at room temperature for 16 hours under an atmosphere of hydrogen. The reaction mixture was filtered through a Celite® plug and the filtrate was concentrated under reduced pressure to give [(2S,6S)-6-(difluoromethyl)morpholin-2-yl]methanol as a pale yellow oil.

To a solution of [(2S,6S)-6-(difluoromethyl)morpholin-2-yl]methanol (340 mg, 2.03 mmol) in dichloromethane (8 mL) cooled to 0° C., was added an aqueous solution of sodium hydroxide (1M, 2.0 mL, 2.03 mmol) and the reaction mixture was stirred for 10 minutes before addition of di-tert-butyl dicarbonate (488 mg, 2.24 mmol) and the reaction mixture was stirred at room temperature for 16 hours. The reaction mixture was partitioned between water and dichloromethane. The aqueous layer was further extracted with dichloromethane and the combined organic layer was dried over anhydrous sodium sulfate and concentrated under reduced pressure. The crude material was purified by column chromatography on silica gel eluting with 25% ethyl acetate in hexane to give tert-butyl (2S,6S)-2-(difluoromethyl)-6-(hydroxymethyl)morpholine-4-carboxylate (300 mg, 1.10 mmol, 47% yield) as a colourless oil. 1H NMR (400 MHz, DMSO-d6) δ 6.06 (td, 1H), 4.89 (t, 1H), 4.05-3.67 (m, 3H), 3.54-3.36 (m, 3H), 2.93-2.58 (m, 2H), 1.41 (s, 9H). LCMS (Method 8-Column 1): Retention Time=7.29 minutes, [MH]+=268.

### Synthesis of [(2S,6R)-4-benzyl-6-(trifluoromethyl)morpholin-2-yl]methanol and [(2S,6S)-4-benzyl-6-(trifluoromethyl)morpholin-2-yl]methanol

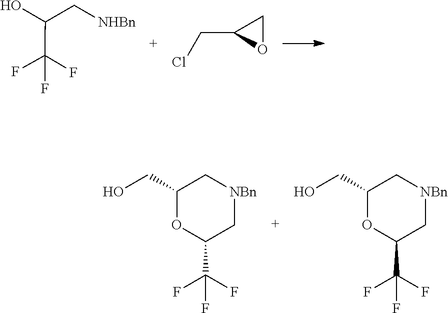

To a solution of 3-(benzylamino)-1,1,1-trifluoropropan-2-ol (3.2 g, 14.6 mmol) in toluene (32 mL) was added dropwise (R)-(−)-epichlorohydrin (2.0 g, 21.9 mmol) followed by portionwise addition of lithium perchlorate (1.6 g, 14.6 mmol). The reaction was stirred at room temperature for 16 hours. After 16 hours was added dropwise sodium methoxide (30% solution in methanol, 8.3 mL, 43.8 mmol) and the reaction mixture was stirred at room temperature for 16 hours. The reaction mixture was partitioned between water and ethyl acetate. The aqueous layer was further extracted with ethyl acetate and the combined organic layer was dried over anhydrous sodium sulfate and concentrated under reduced pressure. The crude material was purified by column chromatography on silica gel eluting with 10-20% ethyl acetate in hexane gradient to give [(2S,6R)-4-benzyl-6-(trifluoromethyl)morpholin-2-yl]methanol (1.0 g, 3.63 mmol, 25% yield) and [(2S,6S)-4-benzyl-6-(trifluoromethyl)morpholin-2-yl]methanol (980 mg, 3.33 mmol, 23% yield).

### [(2S,6R)-4-benzyl-6-(trifluoromethyl)morpholin-2-yl]methanol

1H NMR (400 MHz, DMSO-d6) δ 7.38-7.22 (m, 5H), 4.74 (t, 1H), 4.42-4.16 (m, 1H), 3.94-3.78 (m, 1H), 3.61-3.39 (m, 4H), 2.72-2.51 (m, 3H), 2.28 (dd, 1H). LCMS (Method 8-Column 1): Retention Time=7.66 minutes, [MH]+=276.

### [(2S,6S)-4-benzyl-6-(trifluoromethyl)morpholin-2-yl]methanol

1H NMR (400 MHz, DMSO-d6) δ 7.40-7.24 (m, 5H), 4.82 (t, 1H), 4.29-4.20 (m, 1H), 3.67-3.57 (m, 2H), 3.53 (d, 1H), 3.48-3.40 (m, 1H), 3.34-3.29 (m, 1H), 2.88 (d, 1H), 2.80 (d, 1H), 2.01 (t, 1H), 1.83 (t, 1H). LCMS (Method 8-Column 1): Retention Time=6.89 minutes, [MH]+=276.

### Synthesis of tert-butyl (2S,6R)-2-(hydroxymethyl)-6-(trifluoromethyl)morpholine-4-carboxylate

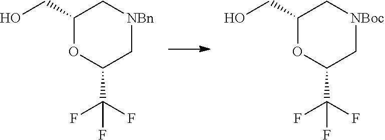 [SMILES: OC[C@@H]1CN(Cc2ccccc2)C[C@H](C(F)(F)F)O1 ; CC(C)(C)OC(=O)N1C[C@@H](CO)O[C@@H](C(F)(F)F)C1]

To a solution of [(2S,6R)-4-benzyl-6-(trifluoromethyl)morpholin-2-yl]methanol (1.0 g, 3.63 mmol) in methanol (20 mL) was added palladium hydroxide on carbon (20% loading wet support, 459 mg, 0.33 mmol) and the reaction mixture was stirred at room temperature for 16 hours under an atmosphere of hydrogen. The reaction mixture was filtered through a Celite® plug and the filtrate was concentrated under reduced pressure to give intermediate [(2S,6R)-6-(trifluoromethyl)morpholin-2-yl]methanol as a pale yellow oil.

To a solution of [(2S,6R)-6-(trifluoromethyl)morpholin-2-yl]methanol (580 mg, 3.13 mmol) in dichloromethane/water (15:5 mL) cooled to 0° C., was added sodium hydroxide (125 mg, 3.13 mmol) and the reaction mixture was stirred for 10 minutes before addition of di-tert-butyl dicarbonate (752 mg, 3.45 mmol) and the reaction mixture was stirred at room temperature for 16 hours. The reaction was partitioned between water and dichloromethane and the aqueous layer was further extracted with dichloromethane. The combined organic layer was dried over anhydrous sodium sulfate and concentrated under reduced pressure. The crude material was purified by column chromatography on silica gel eluting with 15-30% ethyl acetate in hexane to give tert-butyl (2S,6R)-2-(hydroxymethyl)-6-(trifluoromethyl)morpholine-4-carboxylate (700 mg, 2.45 mmol, 67% yield) as a pale yellow oil. 1H NMR (400 MHz, DMSO-d6) δ 4.86 (t, 1H), 4.47-4.30 (m, 1H), 3.90-3.55 (m, 3H), 3.55-3.36 (m, 3H), 3.25-2.88 (m, 1H), 1.40 (s, 9H). LCMS (Method 4-Column 7): Retention Time=1.88 minutes, [(M−^{t}Bu)H]+=230.

### Synthesis of tert-butyl (2S,6S)-2-(hydroxymethyl)-6-(trifluoromethyl)morpholine-4-carboxylate

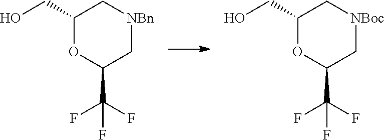 [SMILES: OC[C@@H]1CN(Cc2ccccc2)C[C@@H](C(F)(F)F)O1 ; CC(C)(C)OC(=O)N1C[C@@H](CO)O[C@H](C(F)(F)F)C1]

To a solution of [(2S,6S)-4-benzyl-6-(difluoromethyl)morpholin-2-yl]methanol (980 mg, 3.56 mmol) in methanol (20 mL) was added palladium hydroxide on carbon ((20% loading wet support, 450 mg, 0.32 mmol) and the reaction mixture was stirred at room temperature for 16 hours under an atmosphere of hydrogen. The reaction mixture was filtered through a Celite® plug and the filtrate was concentrated under reduced pressure to give [(2S,6S)-6-(trifluoromethyl)morpholin-2-yl]methanol as a pale yellow oil.

To a solution of [(2S,6S)-6-(trifluoromethyl)morpholin-2-yl]methanol (520 mg, 2.81 mmol) in dichloromethane/water (15:5 mL) cooled to 0° C., was added sodium hydroxide (112 mg, 2.81 mmol) and the reaction mixture was stirred for 10 minutes before addition of di-tert-butyl dicarbonate (674 mg, 3.09 mmol) and the reaction mixture was stirred at room temperature for 16 hours. The reaction mixture was partitioned between water and dichloromethane. The aqueous layer was further extracted with dichloromethane and the combined organic layer was dried over anhydrous sodium sulfate and concentrated under reduced pressure. The crude material was purified by column chromatography on silica gel eluting with 15-30% ethyl acetate in hexane to give tert-butyl (2S,6S)-2-(hydroxymethyl)-6-(trifluoromethyl)morpholine-4-carboxylate (600 mg, 2.10 mmol, 59% yield) as a colourless oil. 1H NMR (400 MHz, DMSO-d6) δ 4.93 (t, 1H), 4.36-4.17 (m, 1H), 4.08-3.81 (m, 2H), 3.60-3.46 (m, 2H), 3.45-3.36 (m, 1H), 2.97-2.61 (m, 2H), 1.42 (s, 9H). LCMS (Method 4-Column 7): Retention Time=1.99 minutes, [MH])=286.

### Synthesis of [(6S)-8-benzyl-2,5-dioxa-8-azaspiro[3.5]nonan-6-yl]methanol

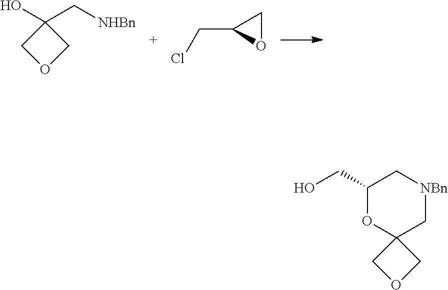

To a solution of 3-[(benzylamino)methyl]oxetan-3-ol (1.4 g, 7.24 mmol) in toluene (15 mL) was added dropwise (R)-(−)-epichlorohydrin (1.0 g, 10.9 mmol) followed by portionwise addition of lithium perchlorate (770 mg, 7.24 mmol). The reaction was stirred at room temperature for 16 hours. After 16 hours was added dropwise sodium methoxide (30% solution in methanol, 4.2 mL, 21.7 mmol) and the reaction mixture was stirred at room temperature for 16 hours. The reaction mixture was partitioned between water and ethyl acetate. The aqueous layer was further extracted with ethyl acetate and the combined organic layer was dried over anhydrous sodium sulfate and concentrated under reduced pressure. The crude material was purified by column chromatography on silica gel eluting with 10-20% ethyl acetate in hexane gradient to give [(6S)-8-benzyl-2,5-dioxa-8-azaspiro[3.5]nonan-6-yl]methanol (1.2 g, 4.57 mmol, 63% yield) as a pale yellow oil. 1H NMR (400 MHz, DMSO-d6) δ 7.45-7.20 (m, 5H), 4.70 (t, 1H), 4.49 (d, 1H), 4.40-4.27 (m, 2H), 4.22 (d, 1H), 3.55-3.49 (m, 1H), 3.48-3.39 (m, 2H), 3.31-3.24 (m, 2H), 3.04 (d, 1H), 2.73 (d, 1H), 2.00 (d, 1H), 1.79 (t, 1H). LCMS (Method 4-Column 7: Retention Time=0.80 minutes, [MH]+=250.

### Synthesis of tert-butyl (6S)-6-(hydroxymethyl)-2,5-dioxa-8-azaspiro[3.5]nonane-8-carboxylate

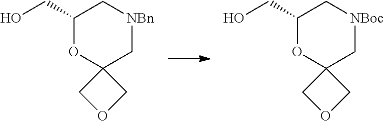 [SMILES: OC[C@@H]1CN(Cc2ccccc2)CC2(COC2)O1 ; CC(C)(C)OC(=O)N1C[C@@H](CO)OC2(COC2)C1]

To a solution of [(6S)-8-benzyl-2,5-dioxa-8-azaspiro[3.5]nonan-6-yl]methanol (1.2 g, 4.81 mmol) in methanol (15 mL) was added palladium hydroxide on carbon (20% loading, 608 mg, 0.87 mmol) and the reaction mixture was stirred at room temperature for 16 hours under an atmosphere of hydrogen. The reaction mixture was filtered through a Celite® plug and the filtrate was concentrated under reduced pressure to give intermediate [(6S)-2,5-dioxa-8-azaspiro[3.5]nonan-6-yl]methanol as a pale yellow oil.

To a solution of [(6S)-2,5-dioxa-8-azaspiro[3.5]nonan-6-yl]methanol (700 mg, 4.40 mmol) in dichloromethane (15 mL): water (5 mL) cooled to 0° C., was added sodium hydroxide (176 mg, 4.40 mmol) and the reaction mixture was stirred for 10 minutes before addition of di-tert-butyl dicarbonate (1.0 g, 4.84 mmol) and the reaction mixture was stirred at room temperature for 16 hours. The reaction was partitioned between water and dichloromethane and the aqueous layer was further extracted with dichloromethane. The combined organic layer was dried over anhydrous sodium sulfate and concentrated under reduced pressure. The crude material was purified by column chromatography on silica gel eluting with 30-40% ethyl acetate in hexane gradient to give tert-butyl (6S)-6-(hydroxymethyl)-2,5-dioxa-8-azaspiro[3.5]nonane-8-carboxylate (780 mg, 3.01 mmol, 63% yield) as a pale yellow oil. 1H NMR (400 MHz, DMSO-d6) δ 4.83 (t, J=5.6 Hz, 1H), 4.47 (d, J=6.6 Hz, 1H), 4.39-4.31 (m, 2H), 4.29 (d, J=6.6 Hz, 1H), 4.18 (d, J=13.6 Hz, 1H), 3.92-3.75 (m, 1H), 3.51-3.41 (m. 1H), 3.39-3.34 (m, 2H), 3.02-2.77 (m, 1H), 2.69-2.54 (m, 1H), 1.42 (s, 9H). LCMS (Method 4-Column 7): Retention Time=1.47 minutes, [MH]+=260.

### Synthesis of rac-(2R)-2-[(2S,4R)-1-benzyl-4-(hydroxymethyl)azetidin-2-yl]-N-tert-butyl-2-hydroxyacetamide

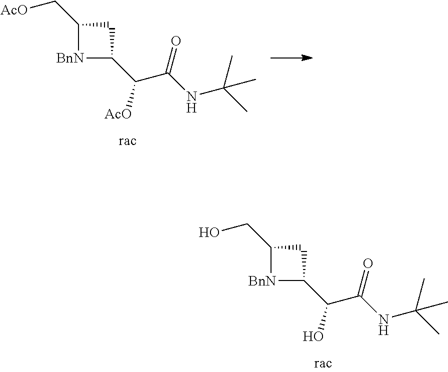 [SMILES: CC(=O)OC[C@@H]1C[C@H]([C@@H](OC(C)=O)C(=O)NC(C)(C)C)N1Cc1ccccc1 ; CC(C)(C)NC(=O)[C@H](O)[C@H]1C[C@@H](CO)N1Cc1ccccc1]

To a solution of [rac-(R)-[(2R,4S)-4-[(acetyloxy)methyl]-1-benzylazetidin-2-yl](tert-butylcarbamoyl)methyl acetate (3.1 g, 7.9 mmol) in methanol (30 mL) cooled to 0° C., was added potassium hydroxide (1.3 g, 23.8 mmol). The reaction mixture was stirred for 1.5 hours at 0° C. The reaction mixture was quenched by addition of 10% aqueous ammonium chloride solution and the products were extracted with ethyl acetate. The combined organic layers were washed with water and dried over anhydrous sodium sulfate. The solvents were moved to give rac-(2R)-2-[(2R,4S)-1-benzyl-4-(hydroxymethyl)azetidin-2-yl]-N-tert-butyl-2-hydroxyacetamide (2.5 g, 8.0 mmol, 101% yield) as off white solid. The crude material was used directly for the next step. 1H NMR (400 MHz, Chloroform-d): δ 7.34-7.29 (m, 5H), 6.59 (br s, 1H), 3.76-3.71 (m, 3H), 3.59 (d, 1H), 3.41-3.40 (m, 2H), 3.37-3.32 (m, 1H), 2.0-1.89 (m, 2H), 1.33 (s, 9H). LCMS (Method 5): Retention Time=1.36 minutes, [MH]+=307.

### Synthesis of rac-(1S,2S,5R)-6-benzyl-N-tert-butyl-3-oxa-6-azabicyclo[3.1.1]heptane-2-carboxamide

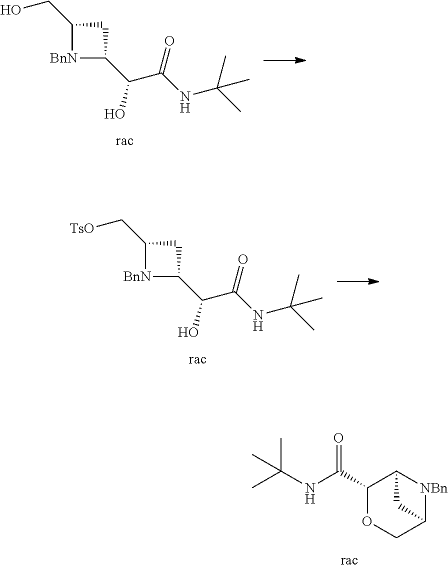 [SMILES: CC(C)(C)NC(=O)[C@H](O)[C@H]1C[C@@H](CO)N1Cc1ccccc1 ; Cc1ccc(S(=O)(=O)OC[C@@H]2C[C@H]([C@@H](O)C(=O)NC(C)(C)C)N2Cc2ccccc2)cc1 ; CC(C)(C)NC(=O)[C@H]1OC[C@H]2C[C@@H]1N2Cc1ccccc1]

To a solution of rac-(2R)-2-[(2R,4S)-1-benzyl-4-(hydroxymethyl)azetidin-2-yl]-N-tert-butyl-2-hydroxyacetamide (2.5 g, 8.03 mmol) in dichloromethane (40 mL) cooled to 0° C., was added 4-dimethylaminopyridine (1 mg, 0.03 mmol) and triethylamine (4.2 mL, 30.1 mmol). Then p-toluenesulfonyl chloride (1.6 g, 8.03 mmol) was added and the reaction mixture was warmed to room temperature and stirred overnight. The reaction mixture was partitioned between 10% aqueous sodium bicarbonate solution and dichloromethane. The aqueous layer was further extracted with dichloromethane and the combined organic layers were washed with water, dried over anhydrous sodium sulfate and concentrated under reduced pressure. The crude material was purified by flash column chromatography eluting with 35% ethyl acetate in petroleum ether to give intermediate [rac-(2S,4R)-1-benzyl-4-[(R)-(tert-butylcarbamoyl)(hydroxy)methyl]azetidin-2-yl]methyl 4-methylbenzene-1-sulfonate (1.0 g, 2.17 mmol, 27% yield) as a pale yellow solid. LCMS (Method 5): Retention Time=1.98 minutes, [MH]+=461.

To a stirred solution of [rac-(2S,4R)-1-benzyl-4-[(R)-(tert-butylcarbamoyl)(hydroxy)methyl]azetidin-2-yl]methyl 4-methylbenzene-1-sulfonate (1.0 g, 2.17 mmol) in N,N-dimethylformamide (25 mL) was added sodium hydride (57-63% oil dispersion, 113 mg, 2.82 mmol). The reaction mixture was stirred at room temperature overnight. The reaction mixture was quenched by addition of 10% aqueous ammonium chloride solution and the products were extracted with ethyl acetate. The combined organic layers were washed with water, dried over anhydrous sodium sulfate and concentrated under reduced pressure. The crude material was purified by flash column chromatography eluting with 22% ethyl acetate in petroleum ether to give rac-(1S,2S,5R)-6-benzyl-N-tert-butyl-3-oxa-6-azabicyclo[3.1.1]heptane-2-carboxamide (494 mg, 1.71 mmol, 80% yield) as a yellow oil. 1H NMR (400 MHz, Chloroform-d) S 7.39-7.37 (m, 2H), 7.34-7.31 (m, 2H), 7.27-7.25 (m, 1H), 6.73 (br s, 1H), 4.56 (d, 1H), 4.33 (d, 1H), 3.93-3.90 (m, 1H), 3.86 (s, 3H), 3.53-3.51 (m, 1H), 2.63-2.61 (m, 1H), 1.64 (d, 1H), 1.39 (s, 9H). LCMS (Method 6): Retention Time=1.56 minutes, [MH]+=289.

### Synthesis of tert-butyl rac-(1S,2S,5R)-2-(hydroxymethyl)-3-oxa-6-azabicyclo[3.1.1]heptane-6-carboxylate

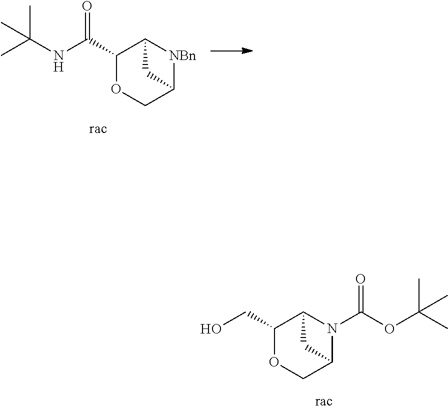 [SMILES: CC(C)(C)NC(=O)[C@H]1OC[C@H]2C[C@@H]1N2Cc1ccccc1 ; CC(C)(C)OC(=O)N1[C@H]2CO[C@H](CO)[C@@H]1C2]

A solution of rac-(1S,2S,5R)-6-benzyl-N-tert-butyl-3-oxa-6-azabicyclo[3.1.1]heptane-2-carboxamide (494 mg, 1.71 mmol) in concentrated hydrochloric acid (49 mL) was stirred at 55° C. for 14 hours. The solvents were removed under reduced pressure to give rac-(1S,2S,5R)-6-benzyl-3-oxa-6-azabicyclo[3.1.1]heptane-2-carboxylic acid, hydrochloride (630 mg, 2.34 mmol) as red hygroscopic solid which was used directly for the next step. LCMS (Method 6): Retention Time=1.01 minutes, [MH]+=234.

To a solution of rac-(1S,2S,5R)-6-benzyl-3-oxa-6-azabicyclo[3.1.1]heptane-2-carboxylic acid, hydrochloride (630 mg, 2.34 mmol) in tetrahydrofuran (25 mL) cooled to 0° C., was added lithium aluminium hydride (2M in tetrahydrofuran, 3.5 mL, 7.01 mmol). The reaction mixture was stirred at 0-10° C. for 2 hours. The reaction mixture was quenched with 10% aqueous ammonium chloride solution and the products were extracted with ethyl acetate. The combined organic layers were washed water, dried over anhydrous sodium sulfate and the volatiles were removed under reduced pressure to give [rac-(1S,2S,5R)-6-benzyl-3-oxa-6-azabicyclo[3.1.1]heptan-2-yl]methanol (295 mg, 1.35 mmol, 58% yield) as red oil.

A solution of [rac-(1S,2S,5R)-6-benzyl-3-oxa-6-azabicyclo[3.1.1]heptan-2-yl]methanol (290 mg, 1.32 mmol) in methanol (30 mL) was degassed with nitrogen for 5 minutes before addition of palladium on carbon (10% w/w, 70 mg). The reaction mixture was placed under an atmosphere of hydrogen, and it was stirred for 16 hours. The reaction mass was filtered through a pad of Celite® washing with methanol. The combined filtrate concentrated under reduced pressure to give [rac-(1S,2S,5R)-3-oxa-6-azabicyclo[3.1.1]heptan-2-yl]methanol (145 mg, 1.12 mmol, 85% yield) as a brown oil. LCMS (Method 5): Retention Time=0.56 minutes, [MH]+=130.

To a solution of [rac-(1S,2S,5R)-3-oxa-6-azabicyclo[3.1.1]heptan-2-yl]methanol (140 mg, 1.08 mmol) in tetrahydrofuran (5 mL) was added triethylamine (180 μL, 1.30 mmol) and 4-dimethylaminopyridine (14 mg, 0.11 mmol). The reaction mixture was cooled 0° C. and was added di-tert-butyl dicarbonate (236 mg, 1.08 mmol). The reaction mixture was stirred at room temperature overnight. The reaction mixture was partitioned between water and dichloromethane, and the aqueous layer was further extracted with dichloromethane. The combined organic layer was washed with water and dried over anhydrous sodium sulfate. The crude material was purified by flash column chromatography on silica gel, eluting with 35% ethyl acetate in petroleum ether to give tert-butyl rac-(1S,2S,5R)-2-(hydroxymethyl)-3-oxa-6-azabicyclo[3.1.1]heptane-6-carboxylate (100 mg, 0.44 mmol, 40% yield) as light brown oil. 1H NMR (400 MHz, Chloroform-d) δ 4.40-4.15 (m, 2H), 4.15-3.93 (m, 2H), 3.83-3.75 (m, 1H), 3.67-3.56 (m, 2H), 2.53-2.44 (m, 1H), 1.93-1.81 (m, 2H), 1.47 (s, 9H). LCMS (Method 6): Retention Time=1.51 minutes, [(M−Boc)H]+=130.

### Biological Assays

### Syk Biochemical Assay

Compounds were serially diluted in 100% DMSO, prior to an intermediate dilution in water. In a low volume white 384-well Proxiplate (Perkin Elmer), 1 μL of compound (0.4% final DMSO) was added to 3 μL of 10 ng/well Syk (Promega) and 0.2 mg/mL Poly-E4Y1 substrate (Promega) in assay buffer (final 40 mM Tris-HCl pH 7.4, 20 mM MgCl₂, 0.1 mg/mL BSA) for 5 minutes. The reaction was initiated by addition of 1 μL of ATP (40 μM final) and allowed to proceed for 60 minutes at room temperature. The reaction was terminated by addition of 5 μL of ADP-Glo Reagent Mix (Promega) containing 1 mM MgCl₂ for 45 minutes at room temperature. 10 μL of Kinase Detection Reagent (Promega) was added and incubated for 10 minutes at room temperature, before luminescence was measured using an Ensight Multimode Plate Reader (Perkin Elmer).

### Ba/F3 Proliferation Assay

Ba/F3-Syk and Ba/F3-WT (wild type) cells were maintained at ≤1×10⁶ cell/mL in RPMI (Life Technologies) with 10% FBS at 37° C. and 5% CO₂ for a maximum of four weeks. Ba/F3-WT additionally contained 2 ng/mL mIL-3 (R&D Systems) to induce cellular growth. Cells were counted and dispensed at 5000 cells/well using a Multidrop Liquid Dispenser in a volume of 45 μL into black 384-well TC treated plates (Greiner) and incubated for four hours in complete media (+mIL-3 for Ba/F3-WT). Compounds were serially diluted in 100% DMSO, prior to an intermediate dilution in RPMI media. 5 μL of compound (0.4% final DMSO) was added to the cells, followed by a 48-hour incubation period. 5 μL of Resazurin (60 μM final) was then added and cells were incubated at 37° C. for three hours, before fluorescence (λex530/λem585) was measured using an Ensight Multimode Plate Reader (Perkin Elmer).

TABLE 16
Biological Results

| | SYK-Ba/F3 | WT-Ba/F3 |
|---|---|---|
| No | EC₅₀ (nM) | EC₅₀ (nM) |
| 2.1 | 92.8 | 1250 |
| 2.2 | 355.3 | 1760 |
| 2.3 | 104.5 | 2350 |
| 2.5 | 76.7 | 2539 |
| 2.6 | 57.2 | 1533 |
| 2.7 | 37.6 | 933.9 |
| 2.8 | 195.6 | 1436 |
| 2.9 | 186.6 | 2356 |
| 2.10 | 39.9 | 2784 |
| 2.11 | 130.7 | — |
| 2.13 | 79 | 2404 |
| 2.14 | 3622 | 4828 |
| 2.15 | 139.5 | 2887 |
| 2.16 | 492.7 | >10000 |
| 2.17 | 2459 | >10000 |
| 2.18 | 78.2 | 1861 |
| 2.19 | 198 | 2784 |
| 2.20 | 87.5 | 4171 |
| 2.21 | 61 | 2697 |
| 2.22 | 30.6 | 693.7 |
| 2.23 | 440.3 | 5804 |
| 2.24 | 134,9 | 1503 |
| 2.25 | 62.1 | 1509 |
| 2.26 | 216.3 | 1447 |
| 2.27 | 305.2 | 1503 |
| 2.28 | 733.5 | >10000 |
| 2.29 | 1020 | >10000 |
| 2.30 | 205.9 | 6968 |
| 2.31 | 1738 | >10000 |
| 2.32 | 48.4 | 4758 |
| 2.33 | 390.5 | 1588 |
| 2.34 | 394.3 | 1563 |
| 2.35 | 667.1 | 618.9 |
| 2.36 | 279 | 2053 |
| 2.37 | 1229 | 2833 |
| 2.38 | 1447 | >10000 |
| 2.39 | 243.2 | 1703 |
| 2.40 | 644.7 | >10000 |
| 2.41 | 126.9 | 2325 |
| 2.42 | 12.5 | 2340 |
| 2.43 | 79.9 | 2519 |
| 2.44 | 53.1 | 2112 |
| 2.45 | 17.8 | 1476 |
| 2.46 | 65 | 3429 |
| 2.47 | 48 | 3267 |
| 2.48 | 119 | 4059 |
| 2.49 | 47.1 | 3047 |
| 2.50 | 151.2 | 6183 |
| 2.51 | 45.5 | 6066 |
| 2.52 | 57.4 | 9828 |
| 2.53 | 128.6 | 2556 |
| 2.54 | 288.1 | 1580 |
| 2.55 | 43.8 | 3270 |
| 2.56 | 32.9 | 1459 |
| 2.57 | 21.6 | >10000 |
| 2.58 | 25.4 | 6712 |
| 2.59 | 58.9 | 984.8 |
| 2.60 | 26.6 | 4071 |
| 2.61 | 9.7 | 2614 |
| 2.62 | 31.4 | 1291 |
| 2.63 | 20.6 | 2870 |
| 2.64 | 253 | >10000 |
| 2.65 | 11.7 | 2535 |
| 2.66 | 14.5 | 1024 |
| 2.67 | 18.6 | 1859 |
| 2.68 | 13.9 | 4372 |
| 2.69 | 13.5 | >10000 |
| 2.70 | 19.1 | 1544 |
| 2.71 | 1328 | >10000 |
| 2.72 | 265.9 | 1902 |
| 2.73 | 32.2 | 3378 |
| 2.74 | 109.1 | 2885 |
| 2.75 | 67.8 | 3924 |
| 2.76 | 44.8 | 2763 |
| 2.77 | 42.7 | 5316 |
| 2.78 | 147.3 | 4854 |
| 2.79 | 595.9 | >10000 |
| 2.80 | 36 | 1603 |
| 2.81 | 73.2 | 2066 |
| 2.82 | 126.9 | 2267 |
| 2.83 | 34.2 | 1646 |
| 2.84 | 22.3 | 2209 |
| 2.85 | 59.3 | 9099 |
| 2.86 | 2160 | >10000 |
| 2.87 | 11.2 | 1098 |
| 2.88 | 17.6 | 1556 |
| 2.89 | 21.7 | 2727 |
| 2.90 | 54.6 | >1000 |
| 2.91 | 51.8 | 1041 |
| 2.92 | 29 | 598.7 |
| 2.93 | 27.7 | 1240 |
| 2.94 | 145.3 | 1422 |
| 2.95 | 22.13 | 1854 |
| 2.96 | 20.1 | 723.6 |
| 2.97 | 347.4 | 4240 |
| 2.98 | 18.6 | 845.5 |
| 2.99 | 17.8 | 1008 |
| 2.100 | 361.9 | 3008 |
| 2.101 | 25.4 | 628.1 |
| 2.102 | 11.7 | 1074 |
| 2.103 | 32.7 | 984.7 |
| 2.104 | 33.1 | 1570 |
| 2.105 | 31.1 | 1548 |
| 2.106 | 55.5 | 575.4 |
| 3.1 | 78.2 | 2655 |
| 3.2 | 23.5 | 2372 |
| 3.3 | 178.9 | 2593 |
| 3.4 | 47.4 | 925.9 |
| 3.5 | 61.8 | 901.5 |
| 3.6 | 23.3 | 2122 |
| 3.7 | 134.2 | 3731 |
| 3.8 | 50.9 | 1971 |
| 3.9 | 167.1 | >10000 |
| 3.10 | 53.2 | 4609 |
| 3.11 | 163 | 2140 |
| 3.12 | 87 | 1682 |
| 3.13 | 35.8 | 813.9 |
| 3.14 | 54.5 | 6494 |
| 3.15 | 30.4 | 5843 |
| 3.16 | 11.9 | 6172 |
| 3.17 | 51.8 | 2224 |
| 3.18 | 23.1 | 5420 |
| 3.19 | 99.1 | 6799 |
| 3.20 | 35.5 | 1959 |
| 3.21 | 13.1 | 614.5 |
| 3.22 | 99 | 9675 |
| 3.23 | 12.1 | 941.2 |
| 3.24 | 28.2 | 2261 |
| 4.1 | 2322 | 7432 |
| 4.2 | 171.3 | >10000 |
| 4.3 | 585.5 | 5006 |
| 6.1 | 38.8 | 2658 |
| 6.2 | 35.6 | 2554 |
| 6.3 | 51.9 | 2455 |
| 6.4 | 74.8 | 2647 |
| 6.5 | 63 | 2135 |
| 6.6 | 81.9 | 1916 |
| 6.7 | 113.2 | 3397 |
| 6.8 | 180.8 | 4879 |
| 6.9 | 11.4 | 3295 |
| 6.10 | 105.9 | 1976 |
| 6.11 | 30.7 | 1681 |
| 6.12 | 66.7 | 994.5 |
| 6.13 | 21.8 | 2051 |
| 6.14 | 14.3 | 1803 |
| 6.15 | 20.3 | 2337 |
| 6.16 | 41.9 | 3119 |
| 6.17 | 79.9 | 2400 |
| 6.18 | 36.2 | 3286 |
| 6.19 | 21.4 | 5015 |
| 6.20 | 32.1 | 2177 |
| 6.21 | 12.6 | 1513 |
| 6.22 | 11.2 | 2092 |
| 6.23 | 28.4 | 2100 |
| 6.24 | 16.4 | 2816 |
| 6.25 | 8.6 | 1762 |
| 6.26 | 21.4 | >10000 |
| 6.27 | 52.2 | 5594 |
| 6.28 | 15 | 2677 |
| 6.29 | 30.7 | 1441 |
| 6.30 | 27.7 | 1392 |
| 6.31 | 49.4 | 3345 |
| 6.32 | 92 | 2019 |
| 6.33 | 49.3 | 3756 |
| 6.34 | 91.8 | 2082 |
| 6.35 | 23.6 | 2298 |
| 6.36 | 81.6 | 1388.1 |
| 6.37 | 60.3 | 560 |
| 6.38 | 25.9 | 972.1 |
| 6.39 | 21.4 | 1517 |
| 6.40 | 59.8 | 1439 |
| 6.42 | 15.1 | 838.1 |
| 6.43 | 14.9 | >1000 |
| 6.44 | 32.9 | >1000 |
| 6.45 | 68.7 | >1000 |
| 7.1 | 47.9 | 2777 |
| 7.2 | 54.6 | 2109 |
| 7.3 | 74 | 2877 |
| 7.4 | 29.7 | 2693 |
| 7.5 | 36 | 2096 |
| 7.6 | 28.5 | 1771 |
| 7.7 | 86.5 | 4327 |
| 7.8 | 19.3 | 3270 |
| 7.9 | 233.7 | 2439 |
| 7.10 | 44.7 | 3877 |
| 7.11 | 14.4 | 1695 |
| 7.12 | 23.3 | 1563 |
| 7.13 | 22.4 | 4015 |
| 7.14 | 22.5 | 3206 |
| 7.15 | 97.5 | 2766 |
| 7.16 | 19.8 | 1711 |
| 7.17 | 15.8 | 1965 |
| 7.18 | 53.7 | 1747 |
| 7.19 | 36.3 | 1870 |
| 7.20 | 40 | 1686 |
| 7.2 | 24.7 | 2485 |
| 8.1 | 29.3 | 1849 |
| 8.2 | 22.1 | 613.7 |
| 8.3 | 5.6 | 833 |
| 8.4 | 8.2 | 644.9 |
| 8.5 | 4.5 | 735.3 |
| 8.6 | 6.97 | 968.7 |
| 8.7 | 15.1 | 620.6 |
| 8.8 | 16.2 | 1274 |
| 8.9 | 20.5 | 1617 |
| 8.10 | 10.7 | 1622 |
| 8.11 | 6.32 | 625.3 |
| 8.12 | 7.8 | 537.1 |
| 8.13 | 7.3 | 371.2 |
| 8.14 | 8.3 | 434.6 |
| 8.15 | 8.9 | 358.1 |
| 8.16 | 9.4 | 822.2 |
| 8.17 | 4.3 | 684.2 |
| 8.18 | 8.4 | 334.7 |
| 8.19 | 6.4 | 871.8 |
| 8.20 | 27.6 | 1861 |
| 8.21 | 10 | 951.2 |
| 8.22 | 12.6 | 654.7 |
| 8.23 | 2.7 | 420.6 |
| 8.24 | 12.9 | >1000 |
| 8.25 | 11.5 | >1000 |
| 10.1 | 487.1 | >10000 |
| 10.2 | 839.1 | >10000 |
| 10.3 | 365.3 | >10000 |
| 10.4 | 663.5 | 8094 |
| 10.5 | 797.6 | 3279 |
| 10.6 | 106.5 | 754.4 |
| 10.7 | 273.8 | 1198 |
| 10.8 | 81.1 | 1904 |
| 10.9 | 147.9 | 7042 |
| 10.10 | 271.1 | 3679 |
| 10.11 | 80.8 | 4140 |
| 10.12 | 86.1 | 1918 |
| 10.13 | 90.9 | 3210 |
| 10.14 | 281 | 2551 |
| 10.15 | 92.2 | 1678 |
| 10.16 | 259 | 1207 |
| 11.1 | 1356 | >10000 |
| 11.2 | 636.7 | 8458 |
| 11.3 | 37.5 | 557.5 |
| 11.4 | 69.7 | 739.8 |
| 11.5 | 90.6 | 1849 |
| 11.6 | 33.4 | 1787 |
| 11.7 | 46.3 | 1073 |
| 11.8 | 7.4 | 550.6 |
| 11.9 | 10 | 1579 |
| 11.10 | 120.9 | 1696 |
| 12.1 | 213.5 | >10000 |
| 12.2 | 29.1 | 1621 |
| 12.3 | 27.3 | 1300 |
| 12.4 | 27.5 | 3074 |
| 12.5 | 50.9 | 1301 |
| 12.6 | 9.9 | 477.9 |
| 12.7 | 9.2 | 989 |
| 12.8 | 1.3 | 702.6 |
| 13.1 | 585.8 | 6358 |
| 13.3 | 1214 | >10000 |
| 13.4 | 213.9 | 5752 |
| 13.5 | 51.7 | 6313 |
| 13.6 | 36.2 | 5557 |
| 13.7 | 714.4 | 1874.1 |
| 13.8 | 238.4 | 5419 |
| 13.9 | 375.5 | 532 |
| 13.10 | 506.5 | 737.3 |
| 13.11 | 1943 | >10000 |
| 13.12 | 74.4 | >5000 |
| 13.13 | 41.8 | 1778 |
| 13.14 | 233.1 | 1377 |
| 13.15 | 47.2 | 1909 |
| 13.16 | 43.0 | 518.5 |
| 14.1 | 1340 | >10000 |
| 14.2 | 81.8 | 1881 |
| 14.3 | 176.4 | 1925 |
| 14.4 | 196.1 | 1902 |
| 14.5 | 428.4 | 1196 |
| 14.6 | 6.1 | 2673 |
| 14.7 | 2.4 | 930.1 |
| 14.8 | 1170 | >10000 |
| 14.9 | 5.9 | 1219 |
| 14.10 | 44.9 | 1069 |
| 14.11 | 6.1 | 803.1 |
| 14.12 | 5.5 | 866.9 |
| 14.13 | 20 | 864.9 |
| 14.14 | 20.6 | 1097.4 |
| 14.15 | 44.0 | 519.3 |
| 1 | 44.2 | 4670 |
| 2 | 165.9 | 2430 |
| 3 | 162.3 | 1951 |
| 4 | 87.7 | 7212 |

TABLE 17
Biological results

| No | SYK Biochem IC₅₀ (nM) |
|---|---|
| 2.1 | 50.8 |
| 2.2 | 130.5 |
| 2.3 | 39 |
| 2.5 | 38.9 |
| 2.6 | 15.6 |
| 2.7 | 12.8 |
| 2.8 | 37.4 |
| 2.9 | 40.6 |
| 2.10 | 14.5 |
| 2.11 | 18.2 |
| 2.13 | 51.6 |
| 2.15 | 74.8 |
| 2.16 | 222.3 |
| 2.17 | 789 |
| 2.18 | 47.2 |
| 2.19 | 68.5 |
| 2.20 | 37.8 |
| 2.21 | 82.8 |
| 2.22 | 28.1 |
| 2.23 | 106.5 |
| 2.24 | 128.5 |
| 2.25 | 39 |
| 2.26 | 131.4 |
| 2.27 | 246.6 |
| 2.32 | 23.5 |
| 2.42 | 12.2 |
| 2.43 | 31.9 |
| 2.44 | 31 |
| 2.45 | 9.2 |
| 2.46 | 19 |
| 2.49 | 71 |
| 2.51 | 8.1 |
| 2.52 | 22.1 |
| 2.55 | 29.7 |
| 2.57 | 10 |
| 2.58 | 9.6 |
| 2.60 | 24.6 |
| 2.63 | 26.4 |
| 2.67 | 29.5 |
| 2.69 | 30.1 |
| 2.76 | 17.6 |
| 2.98 | 28.3 |
| 2.99 | 21.4 |
| 2.100 | 278 |
| 2.101 | 40 |
| 2.102 | 12.9 |
| 2.103 | 30.5 |
| 2.104 | 16.6 |
| 2.105 | 6.21 |
| 2.106 | 23.6 |
| 3.1 | 48.1 |
| 3.2 | 11.8 |
| 3.4 | 29.3 |
| 3.6 | 10.6 |
| 3.8 | 23.7 |
| 3.10 | 35 |
| 3.14 | 57.1 |
| 3.15 | 39.6 |
| 3.17 | 43.7 |
| 3.19 | 51.1 |
| 3.23 | 12.6 |
| 3.24 | 16.4 |
| 6.2 | 16 |
| 6.3 | 25.6 |
| 6.5 | 14.2 |
| 6.9 | 14.1 |
| 6.10 | 21.3 |
| 6.11 | 24.6 |
| 6.13 | 14.6 |
| 6.16 | 28.4 |
| 6.19 | 11.9 |
| 6.20 | 9.1 |
| 6.22 | 14.3 |
| 6.23 | 55.9 |
| 6.25 | 20.5 |
| 6.26 | 32.1 |
| 6.28 | 41.8 |
| 6.30 | 14.1 |
| 7.2 | 10.1 |
| 7.6 | 17.6 |
| 7.10 | 36.7 |
| 7.11 | 9.7 |
| 7.12 | 29.3 |
| 7.14 | 51.2 |
| 7.21 | 25.6 |
| 8.3 | 5.7 |
| 8.4 | 7.4 |
| 8.5 | 5.2 |
| 8.6 | 10.8 |
| 8.8 | 11.2 |
| 8.10 | 11.8 |
| 8.17 | 14.7 |
| 8.19 | 6.5 |
| 10.1 | 209.4 |
| 11.8 | 14.7 |
| 12.8 | 5.1 |
| 12.40 | 12.1 |
| 13.13 | 16.3 |
| 13.14 | 35.0 |
| 13.15 | 19.8 |
| 13.16 | 31.7 |
| 14.6 | 12.9 |
| 14.14 | 33.6 |
| 14.15 | 21.1 |

### Permeability Assay

The assay was performed using Charles River/Agilux default assay conditions and default acetonitrile extraction and bioanalytical conditions. The assay was performed using MDCKII-MDR1 (Pgp) cells (canine MDCK stably transfected with MDR1). Bi-directional (A-B and B-A) flux measurements were carried out in triplicate aliquots from duplicate wells. The compounds were incubated for 2 hours at 37° C. at a concentration of 10 PM. The efflux ratio is the ratio of PₐₚₚB-A/PₐₚₚA-B, and a ratio >2 indicates the compound is effluxed.

Selected compounds of the invention tested for permeability using MDCKII-MDR1 cells demonstrated a permeability in the range of 15-20×10⁻⁶ cm/s (3.1, 3.6, 6.9) and a permeability greater than 20×10⁻⁶ cm/s (2.20, 2.42, 6.2). Selected compounds of the invention tested for permeability using MDCKII-MDR1 cells demonstrated an efflux ratio in the range of 1-2 (3.6, 6.2) and an efflux ratio of 1 or less (2.20, 2.42, 3.1, 6.9).

### Brain Penetration Assays

### Mouse Pharmacokinetics

Male CD-1 mice (6-8 weeks of age, 3 animals per timepoint) were dosed via oral gavage (10 mL/kg) with compound (10 mg/kg, 0.5% methylcellulose in water). Blood samples were taken either non-terminal via saphenous vein or terminal via cardiac puncture and stored in 1.5 mL Eppendorf tubes containing 0.010 mL of 10% K2EDTA, mixed gently and placed in ice before centrifugation. Blood was centrifuged at 10,000 r.p.m. for 2 minutes, plasma harvested and stored at −80° C. Following blood collection at the terminal time points, animals were sacrificed with C02 and brains quickly removed, rinsed with cold saline (0.9% NaCl), surface vasculature ruptured, blotted with dry gauze or tissue and weighed. The whole brain was homogenized in 1.0 mL ice cold phosphate-buffered saline, pH 7.4 and the homogenates stored at −80° C. Compound concentrations were quantified in plasma and brain tissue by LC-MS/MS using a partially validated bioanalytical method.

### Mouse Plasma Protein Binding

The Rapid Equilibrium Dialysis (RED) method was employed with plasma (200 μL, BioIVT, USA) in phosphate buffered saline (PBS, 350 μL) incubated with compound (1 μM) at 37° C. for 5 hours shaking at 450 r.p.m. Acetonitrile (3 volumes) was added and the sample centrifuged for 5 minutes and 1050 r.p.m. then for 15 minutes at 4000 r.p.m. Compound concentrations were quantified via LC-MS/MS from supernatant and free drug was calculated.

### Mouse Brain Tissue Binding

The RED method was employed with mouse brain homogenate (10% w/v prepared in-house)) incubated with compound (1 μM) at 37° C. for 5 hours shaking at 450 r.p.m. Acetonitrile (3 volumes) was added and the sample centrifuged for 5 minutes and 1050 r.p.m. then for 14 minutes at 4000 r.p.m. Compound concentrations were quantified via LC-MS/MS from supernatant and free drug was calculated.

Selected compounds of the invention demonstrated free brain levels in mice at 1 hour post oral dose of 10 mg/kg of up to 10 nM (2.44, 6.26, 6.28, 7.11, 14.06) of up to 25 nM (2.51, 2.66, 2.67, 3.01, 3.6, 6.02, 6.09, 14.02) and of greater than 25 nM (2.20, 2.42, 2.55, 3.10, 6.19). Selected compounds of the invention demonstrated K_{pu,u} (ratio of unbound brain levels to unbound plasma levels) in mice at 1 hour post oral dose of 10 mg/kg in the range of 0.05-0.1 (2.55, 2.66, 3.6, 14.02), in the range of 0.1-0.2 (2.42, 2.67, 3.1, 6.2, 6.9, 6.19, 6.26), in the range of 0.2-0.3 (2.20, 3.10, 6.28, 7.11) and of greater than 0.3 (2.44, 2.51).

In compliance with the statute, the invention has been described in language more or less specific to structural or methodical features. It is to be understood that the invention is not limited to specific features shown or described since the means herein described comprises preferred forms of putting the invention into effect. The invention is, therefore, claimed in any of its forms or modifications within the proper scope of the appended claims appropriately interpreted by those skilled in the art.

### REFERENCES

Andre, Patrick, Toshifumi Morooka, Derek Sim, Keith Abe, Clifford Lowell, Nisha Nanda, Suzanne Delaney, Gail Siu, Yibing Yan, Stan Hollenbach, Anjali Pandey, Huiyun Gao, Yunmei Wang, Kohsuke Nakajima, Sahil A. Parikh, Can Shi, David Phillips, Whyte Owen, Uma Sinha and Daniel I. Simon. 2011. “Critical role for Syk in responses to vascular injury.” Blood 118 (18): 5000-10. https://doi: 10.1182/blood-2011-06-360743
Baecher-Allan, Clare, Belinda J. Kaskow, and Howard L. Weiner. 2018. “Multiple Sclerosis: Mechanisms and Immunotherapy.” Neuron 97 (4): 742-68. https://doi.org/10.1016/j.neuron.2018.01.021.
Black, Morgan, Farhad Ghasemi, Ren X. Sun, William Stecho, Alessandro Datti, Jalna Meens, Nicole Pinto, et al. 2020. “Spleen Tyrosine Kinase Expression Is Correlated with Human Papillomavirus in Head and Neck Cancer.” Oral Oncology 101: 104529. https://doi.org/10.1016/j.oraloncology.2019.104529.
Braegelmann, Christine, Michael Holzel, Valerie Ludbrook, Marion Dickson, Nil Turan, Sandra Ferring-Schmitt, Sonja Sternberg, Thomas Bieber, Annegret Kuhn, and Joerg Wenzel. 2016. “Spleen Tyrosine Kinase (SYK) Is a Potential Target for the Treatment of Cutaneous Lupus Erythematosus Patients.” Experimental Dermatology 25 (5): 375-79. https://doi.org/10.111/exd.12986.
Bukong, Terence N., Arvin Iracheta-Vellve, Banishree Saha, Aditya Ambade, Abhishek Satishchandran, Benedek Gyongyosi, Patrick Lowe, Donna Catalano, Karen Kodys, and Gyongyi Szabo. 2016. “Inhibition of Spleen Tyrosine Kinase Activation Ameliorates Inflammation, Cell Death, and Steatosis in Alcoholic Liver Disease.” Hepatology (Baltimore, Md.) 64 (4): 1057-71. https://doi.org/10.1002/hep.28680.
Bussel, James, Donald M. Arnold, Elliot Grossbard, Jiri Mayer, Jacek Trelinski, Wojciech Homenda, Andrzej Hellmann, et al. 2018. “Fostamatinib for the Treatment of Adult Persistent and Chronic Immune Thrombocytopenia: Results of Two Phase 3, Randomized, Placebo-Controlled Trials.” American Journal of Hematology 93 (7): 921-30. https://doi.org/10.1002/ajh.25125.
Coffey, Greg, Francis DeGuzman, Mayuko Inagaki, Yvonne Pak, Suzanne M. Delaney, Dan Ives, Andreas Betz, et al. 2012. “Specific Inhibition of Spleen Tyrosine Kinase Suppresses Leukocyte Immune Function and Inflammation in Animal Models of Rheumatoid Arthritis.” The Journal of Pharmacology and Experimental Therapeutics 340 (2): 350-59. https://doi.org/10.1124/jpet.111.188441.
Currie, Kevin S., Jeffrey E. Kropf, Tony Lee, Peter Blomgren, Jianjun Xu, Zhongdong Zhao, Steve Gallion, et al. 2014. “Discovery of GS-9973, a Selective and Orally Efficacious Inhibitor of Spleen Tyrosine Kinase.” Journal of Medicinal Chemistry 57 (9): 3856-73. https://doi.org/10.1021/jm500228a.
Disanto, Giulio, Geir Kjetil Sandve, Vito A. G. Ricigliano, Julia Pakpoor, Antonio J. Berlanga-Taylor, Adam E. Handel, Jens Kuhle, et al. 2014. “DNase Hypersensitive Sites and Association with Multiple Sclerosis.” Human Molecular Genetics 23 (4): 942-48. https://doi.org/10.1093/hmg/ddt489.
Fan, Yannan, Ziqian Zhang, Chunsuo Yao, Jinye Bai, Hui Yang, Pei Ma, Yiyao Fan, et al. 2019. “Amurensin H, a Derivative From Resveratrol, Ameliorates Lipopolysaccharide/Cigarette Smoke-Induced Airway Inflammation by Blocking the Syk/NF-KB Pathway.” Frontiers in Pharmacology 10: 1157. https://doi.org/10.3389/fphar.2019.01157.
Fuller, James P., Jeffrey B. Stavenhagen, and Jessica L. Teeling. 2014. “New Roles for Fc Receptors in Neurodegeneration—the Impact on Immunotherapy for Alzheimer's Disease.” Frontiers in Neuroscience 8. https://doi.org/10.3389/fnins.2014.00235.
Geahlen, Robert L. 2014. “Getting Syk Spleen Tyrosine Kinase as a Therapeutic Target.” Trends in Pharmacological Sciences 35 (8): 414-22. https://doi.org/10.1016/j.tips.2014.05.007.
Ghosh, Soumitra, and Robert L. Geahlen. 2015. “Stress Granules Modulate SYK to Cause Microglial Cell Dysfunction in Alzheimer's Disease.” EBioMedicine 2 (11): 1785-98. https://doi.org/10.1016/j.ebiom.2015.09.053.
Grammatikos, Alexandros P., Debjani Ghosh, Amy Devlin, Vasileios C. Kyttaris, and George C. Tsokos. 2013. “Spleen Tyrosine Kinase (Syk) Regulates Systemic Lupus Erythematosus (SLE) T Cell Signaling.” PLOS ONE 8 (8): e74550. https./doi.org/10.1371/journal.pone.0074550.
Guerreiro, Rita, Aleksandra Wojtas, Jose Bras, Minerva Carrasquillo, Ekaterina Rogaeva, Elisa Majounie, Carlos Cruchaga, et al. 2013. “TREM2 Variants in Alzheimer's Disease.” The New England Journal of Medicine 368 (2): 117-27. https://doi.org/10.1056/NEJMoa1211851.
Hatterer, Eric, Aurelien Benon, Naura Chounlamountri, Chantal Watrin, Julie Angibaud, Emmanuel Jouaneau, Helen Boudin, Jdme Honnorat, Veronique Pellier-Monnin and Nelly Noraz. 2011. “Syk kinase is phosphorylated in specific areas of the developing nervous system.” Neuroscience research 70 (2): 172-82. https://doi:10.1016/j.neures.2011.02.008.
He, Yue, Liang Xu, Bo Li, Zhenni Guo, Qin Hu, Zongduo Guo, Junjia Tang, et al. 2015. “Mincle/Syk Signaling Pathway Contributes to Neuroinflammation after Subarachnoid Hemorrhage in Rats.” Stroke; a Journal of Cerebral Circulation 46 (8): 2277-86. https://doi.org/10.1161/STROKEAHA.115.010088.
Hitchcock, Stephen A., and Lewis D. Pennington. “Structure—brain exposure relationships.” Journal of medicinal chemistry 49, no. 26 (2006): 7559-7583
International Multiple Sclerosis Genetics Consortium, Wellcome Trust Case Control Consortium 2, Stephen Sawcer, Garrett Hellenthal, Matti Pirinen, Chris C. A. Spencer, Nikolaos A. Patsopoulos, et al. 2011. “Genetic Risk and a Primary Role for Cell-Mediated Immune Mechanisms in Multiple Sclerosis.” Nature 476 (7359): 214-19. https://doi.org/10.1038/nature10251.
Kang, Yaqi, Xinrui Jiang, Dalian Qin, Long Wang, Jing Yang, Anguo Wu, Feihong Huang, Yun Ye, and Jianming Wu. 2019. “Efficacy and Safety of Multiple Dosages of Fostamatinib in Adult Patients With Rheumatoid Arthritis: A Systematic Review and Meta-Analysis.” Frontiers in Pharmacology 10 (August). https://doi.org/10.3389/fphar.2019.00897.
Kim, Younyoung, Minyoung Kong, and Chaeyoung Lee. 2013. “Association of Intronic Sequence Variant in the Gene Encoding Spleen Tyrosine Kinase with Susceptibility to Vascular Dementia.” The World Journal of Biological Psychiatry: The Official Journal of the World Federation of Societies of Biological Psychiatry 14 (3): 220-26. https://doi.org/10.3109/15622975.2011.559272.
Kittaka, Mizuho, Tetsuya Yoshimoto, Collin Schlosser, Robert Rottapel, Mikihito Kajiya, Hidemi Kurihara, Ernst J. Reichenberger, and Yasuyoshi Ueki. 2020. “Alveolar Bone Protection by Targeting the SH3BP2-SYK Axis in Osteoclasts.” Journal of Bone and Mineral Research: The Official Journal of the American Society for Bone and Mineral Research 35 (2): 382-95. https://doi.org/10.1002fjbmr.3882.
Kurniawan, Dhadhang Wahyu, Gert Storm, Jai Prakash, and Ruchi Bansal. 2020. “Role of Spleen Tyrosine Kinase in Liver Diseases.” World Journal of Gastroenterology 26 (10): 1005-19. https://doi.org/10.3748/wjg.v26.i10.1005.
Lamb, David J., Stefan Lutz Wollin, Andreas Schnapp, Daniel Bischoff, Klaus J. Erb, Thierry Bouyssou, Bernd Guilliard, et al. 2016. “BI 1002494, a Novel Potent and Selective Oral Spleen Tyrosine Kinase Inhibitor, Displays Differential Potency in Human Basophils and B Cells.” The Journal of Pharmacology and Experimental Therapeutics 357 (3): 554-61. https://doi.org/10.1124/jpet.116.233155.
Lee, Sun-Hwa, and Kyoungho Suk. 2018. “Kinase-Based Taming of Brain Microglia Toward Disease-Modifying Therapy.” Frontiers in Cellular Neuroscience 12 (December). https://doi.org/10.3389/fncel.2018.00474.
Liu, Kang-Han, Nan Zhou, Yan Zou, Yi-Ya Yang, Sha-Xi OuYang, and Yu-Mei Liang. 2019. “Spleen Tyrosine Kinase (SYK) in the Progression of Peritoneal Fibrosis Through Activation of the TGF-B1/Smad3 Signaling Pathway.” Medical Science Monitor: International Medical Journal of Experimental and Clinical Research 25 (December): 9346-56. https://doi.org/10.12659/MSM.917287.
Mielcarska, Matylda B., Magdalena Bossowska-Nowicka, Karolina P. Gregorczyk-Zboroch, Zbigniew Wyzewski, Lidia Szulc-D4browska, Malgorzata Gierynska, and Felix N. Toka. 2019. “Syk and Hrs Regulate TLR3-Mediated Antiviral Response in Murine Astrocytes.” Oxidative Medicine and Cellular Longevity 2019 (April). https://doi.org/10.1155/2019/6927380.
Mócsai, Attila, Jürgen Ruland, and Victor L. J. Tybulewicz. 2010. “The SYK Tyrosine Kinase: A Crucial Player in Diverse Biological Functions.” Nature Reviews Immunology 10 (6): 387-402. https://doi.org/10.1038/nri2765.
Moncayo, Gerald, Michal Grzmil, Tatiana Smirnova, Pawel Zmarz, Roland M. Huber, Debby Hynx, Hubertus Kohler, et al. 2018. “SYK Inhibition Blocks Proliferation and Migration of Glioma Cells and Modifies the Tumor Microenvironment.” Neuro-Oncology 20 (5): 621-31. https://doi.org/10.1093/neuonc/noyOO8.
Munshi, Manit, Xia Liu, Jiaji G. Chen, Lian Xu, Nickolas Tsakmaklis, Maria G. Demos, Amanda Kofides, Maria Luisa Guerrera, Cristina Jimenez, Gloria G. Chan, Zachary R. Hunter, M. Lia Palomba, Kimon V. Argyropoulos, Kirsten Meid, Andrew Keezer, Joshua Gustine, Toni Dubeau, Jorge J. Castillo, Christopher J. Patterson, Jinhua Wang, Sara J. Buhrlage, Nathanael S. Gray, Steven P. Treon and Guang Yang. 2020, “SYK is activated by mutated MYD88 and drives pro-survival signalling in MYD88 driven B-cell lymphomas.” Blood Cancer Journal 10 (1): 12. https://doi: 10.1038/s41408-020-0277-6
Nizami, Sohaib, Hazel Hall-Roberts, Sharat Warrier, Sally A. Cowley, and Elena Di Daniel. 2019. “Microglial Inflammation and Phagocytosis in Alzheimer's Disease: Potential Therapeutic Targets.” British Journal of Pharmacology 176 (18): 3515-32. https://doi.org/10.1111/bph.14618.
Pavel, Ana B., Teresa Song, Hyun-Je Kim, Ester Del Duca, James G. Krueger, Celina Dubin, Xiangyu Peng, Hui Xu, Ning Zhang, Yeriel D. Estrada, Louis Denis, Niranjan Rao, Sandeep Gupta, David J. Zammit, Robert Bissonnette and Emma Guttman-Yassky. 2019. “Oral Janus kinase/SYK inhibition (ASN002) suppresses inflammation and improves epidermal barrier markers in patients with atopic dermatitis.” The Journal of Allergy and Clinical Immunology 144 (4): 1011-24. https://doi:10.1016/j.jaci.2019.07.013
Paris, Daniel, Ghania Ait-Ghezala, Corbin Bachmeier, Gary Laco, David Beaulieu-Abdelahad, Yong Lin, Chao Jin, Fiona Crawford, and Michael Mullan. 2014. “The Spleen Tyrosine Kinase (Syk) Regulates Alzheimer Amyloid-O Production and Tau Hyperphosphorylation.” Journal of Biological Chemistry 289 (49): 33927-44. https://doi.org/10.1074fjbc.M114.608091.
Pine, Polly R., Betty Chang, Nathan Schoettler, Mona L. Banquerigo, Su Wang, Angela Lau, Feifei Zhao, Elliott B. Grossbard, Donald G. Payan, and Ernest Brahn. 2007. “Inflammation and Bone Erosion Are Suppressed in Models of Rheumatoid Arthritis Following Treatment with a Novel Syk Inhibitor.” Clinical Immunology (Orlando, Fla.) 124 (3): 244-57. https://doi.org/10.1016/j.clim.2007.03.543.
Qu, Chen, Dandan Zheng, Sai Li, Yingjun Liu, Anna Lidofsky, Jacinta A. Holmes, Jianning Chen, et al. 2018. “Tyrosine Kinase SYK Is a Potential Therapeutic Target for Liver Fibrosis.” Hepatology (Baltimore, Md.) 68 (3): 1125-39. https://doi.org/10.1002/hep.29881.
Ryu, Jihye, Jeyoung Woo, Jimin Shin, Hyunju Ryoo, Younyoung Kim, and Chaeyoung Lee. 2014. “Profile of Differential Promoter Activity by Nucleotide Substitution at GWAS Signals For Multiple Sclerosis.” Medicine 93 (28). https://doi.org/10.1097/MD.0000000000000281.
Schweig, Jonas Elias, Hailan Yao, David Beaulieu-Abdelahad, Ghania Ait-Ghezala, Benoit Mouzon, Fiona Crawford, Michael Mullan, and Daniel Paris. 2017. “Alzheimer's Disease Pathological Lesions Activate the Spleen Tyrosine Kinase.” Acta Neuropathologica Communications 5 (1): 69. https://doi.org/10.1186/s40478-017-0472-2.
Schweig, Jonas Elias, Hailan Yao, Kyle Coppola, Chao Jin, Fiona Crawford, Michael Mullan, and Daniel Paris. 2019. “Spleen Tyrosine Kinase (SYK) Blocks Autophagic Tau Degradation in Vitro and in Vivo.” Journal of Biological Chemistry 294 (36): 13378-95. https://doi.org/10.1074/jbc.RA119.008033.
Sedlik, Christine, Daniel Orbach, Philippe Veron, Edina Schweighoffer, Francesco Colucci, Romina Gamberale, Andrea Ioan-Facsinay, et al. 2003. “A Critical Role for Syk Protein Tyrosine Kinase in Fc Receptor-Mediated Antigen Presentation and Induction of Dendritic Cell Maturation.” Journal of Immunology (Baltimore, Md.: 1950) 170 (2): 846-52. https://doi.org/10.4049/jimmunol.170.2.846.
Shao, Yuxin, Su Zhang, Yanfen Zhang and Zhongcheng Liu. 2021. “Recent advance of spleen tyrosine kinase in diseases and drugs.” International Immunopharmacology 90: 107168. https://doi.org/10.1016/j.intimp.2020.107168
Sierksma, Annerieke, Ashley Lu, Renzo Mancuso, Nicola Fattorelli, Nicola Thrupp, Evgenia Salta, Jesus Zoco, et al. 2020. “Novel Alzheimer Risk Genes Determine the Microglia Response to Amyloid-P but Not to TAU Pathology.” EMBO Molecular Medicine 12 (3): e10606. https://doi.org/10.15252/emmm.201910606.
Turner, Martin, Edina Schweighoffer, Francesco Colucci, James P Di Santo, and Victor L Tybulewicz. 2000. “Tyrosine Kinase SYK: Essential Functions for Immunoreceptor Signalling.” Immunology Today 21 (3): 148-54. https://doi.org/10.1016/S0167-5699(99)01574-1.
Wager, Travis T., Xinjun Hou, Patrick R Verhoest, and Anabella Villalobos, “Moving beyond Rules: The Development of a Central Nervous System Multiparameter Optimization (CNS MPO) Approach To Enable Alignment of Druglike Properties” ACS Chemical Neuroscience 2010 1 (6), 435-449. DOI: 10.1021/cn100008c
Wex, Eva, Thierry Bouyssou, Matthias J. Duechs, Klaus J. Erb, Florian Gantner, Michael P. Sanderson, Andreas Schnapp, Birgit E. Stierstorfer, and Lutz Wollin. 2011. “Induced Syk Deletion Leads to Suppressed Allergic Responses but Has No Effect on Neutrophil or Monocyte Migration in Vivo.” European Journal of Immunology 41 (11): 3208-18. https://doi.org/10.1002/eji.201141502.
Wong, Brian R., Elliott B. Grossbard, Donald G. Payan, and Esteban S. Masuda. 2004. “Targeting Syk as a Treatment for Allergic and Autoimmune Disorders.” Expert Opinion on Investigational Drugs 13 (7): 743-62. https://doi.org/10.1517/13543784.13.7.743.
Xu, Pengfei, Xiaohao Zhang, Qian Liu, Yi Xie, Xiaolei Shi, Jingjing Chen, Yunzi Li, Hongquan Guo, Rui Sun, Ye Hong, Xinfeng Liu and Gelin Xu. 2019. “Microglia TREM-1 receptor mediates neuroinflammatory injury via interaction with SYK in experimental ischemic stroke.” Cell Death and Disease 10, 555. https://doi.org/10.1038/s41419-019-1777-9.
Ye, Xin-Chun, Qi Hao, Wei-Jing Ma, Qiu-Chen Zhao, Wei-Wei Wang, Han-Han Yin, Tao Zhang, et al. 2020. “Dectin-1/Syk Signaling Triggers Neuroinflammation after Ischemic Stroke in Mice.” Journal of Neuroinflammation 17 (1): 17. https://doi.org/10.1186/s12974-019-1693-z.
Yi, Young-Su, Young-Jin Son, Chongsuk Ryou, Gi-Ho Sung, Jong-Hoon Kim, and Jae Youl Cho. 2014. “Functional Roles of Syk in Macrophage-Mediated Inflammatory Responses.” Review Article. Mediators of Inflammation. Jun. 18, 2014. https://doi.org/10.1155/2014/270302.
Yu, Yu, Yohan Suryo Rahmanto, Yao-An Shen, Laura Ardighieri, Ben Davidson, Stephanie Gaillard, Ayse Ayhan, et al. 2019. “Spleen Tyrosine Kinase Activity Regulates Epidermal Growth Factor Receptor Signaling Pathway in Ovarian Cancer.” EBioMedicine 47 (September): 184-94. https://doi.org/10.1016/j.ebiom.2019.08.055.
Zeng, Ke-Wu, Shu Wang, Xin Dong, Yong Jiang, Hong-Wei Jin, and Peng-Fei Tu. 2014. “Sesquiterpene Dimmer (DSF-27) Inhibits the Release of Neuroinflammatory Mediators from Microglia by Targeting Spleen Tyrosine Kinase (Syk) and Janus Kinase 2 (Jak2): Two Major Non-Receptor Tyrosine Signaling Proteins Involved in Inflammatory Events.” Toxicology and Applied Pharmacology 275 (3): 244-56. https://doi.org/i0.1016/j.taap.2014.01.014.
Zhang, Jinghui, Claudia A. Benavente, Justina McEvoy, Jacqueline Flores-Otero, Li Ding, Xiang Chen, Anatoly Ulyanov, et al. 2012. “A Novel Retinoblastoma Therapy from Genomic and Epigenetic Analyses.” Nature 481 (7381): 329-34. https://doi.org/10.1038/nature10733.

1. A compound of formula (I) or a pharmaceutically acceptable salt or prodrug thereof: 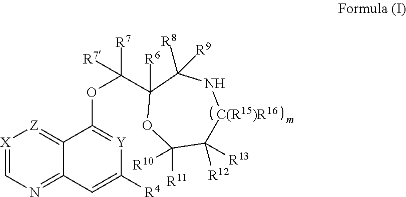
wherein:
Z is CR¹ or N;
Y is CH or N;
X is CR²; and
no more than one of X, Y or Z is N;
wherein:
R¹ is selected from the group consisting of: hydrogen, C₁₋₆alkyl, C₁₋₆fluoroalkyl, C₂₋₆alkenyl, C₂₋₆fluoroalkenyl, C₂₋₆alkynyl, C₂₋₆fluoroalkynyl, C₃₋₆cycloalkyl, halo, —O—C₁₋₆alkyl, —O—C₁₋₆fluoroalkyl, —O—C₂₋₆alkenyl, —O—C₂₋₆fluoroalkenyl, —O—C₂₋₆alkynyl, —O—C₂₋₆fluoroalkynyl and cyano;
R² is selected from the group consisting of: hydrogen, C₁₋₆alkyl, C₁₋₆fluoroalkyl, C₂₋₆alkenyl, C₂₋₆fluoroalkenyl, C₂₋₆alkynyl, C₂₋₆fluoroalkynyl, halo, —O—C₁₋₆alkyl, —O—C₁₋₆fluoroalkyl, —O—C₂₋₆alkenyl, —O—C₂₋₆fluoroalkenyl, —O—C₂₋₆alkynyl, —O—C₂₋₆fluoroalkynyl and cyano;
R⁴ is 5-membered cycloalkene or 5-membered heteroaryl, each of which is optionally fused to form a 5:6, or 5:5 aromatic or heteroaromatic bicycle; wherein each R⁴ is optionally substituted;
m is 0 or 1;
R⁶ is selected from the group consisting of: H, C₁₋₆alkyl, C₁₋₆fluoroalkyl, C₃₋₆cycloalkyl, C₃₋₆fluorocycloalkyl, C₂₋₆alkenyl, C₂₋₆fluoroalkenyl, C₂₋₆alkynyl and C₂₋₆fluoroalkynyl;
R⁷ and R^{7′} are independently selected from the group consisting of: H, fluoro, C₁₋₆alkyl, C₁₋₆fluoroalkyl, C₃₋₆cycloalkyl, C₃₋₆fluorocycloalkyl, C₂₋₆alkenyl, C₂₋₆fluoroalkenyl, C₂₋₆alkynyl and C₂₋₆fluoroalkynyl; or R⁷ and R^{7′} together form a 3 to 6-membered cycloalkyl ring, a 3 to 6-membered fluorocycloalkyl ring, or a 4 to 6-membered oxygen containing heterocyclic ring;
R⁸ and R⁹ are independently selected from the group consisting of: H, C₁₋₆alkyl, C₁₋₆fluoroalkyl, C₃₋₆cycloalkyl, C₃₋₆fluorocycloalkyl, C₂₋₆alkenyl, C₂₋₆fluoroalkenyl, C₂₋₆alkynyl and C₂₋₆fluoroalkynyl; or R⁸ and R⁹ together form a 3 to 6-membered cycloalkyl ring, a 3 to 6-membered fluorocycloalkyl ring, or a 4 to 6-membered oxygen containing heterocyclic ring;
R¹⁰ and R¹¹ are independently selected from the group consisting of: H, C₁₋₆alkyl, fluoro, C₁₋₆fluoroalkyl, C₃₋₆cycloalkyl, C₃₋₆fluorocycloalkyl, C₂₋₆alkenyl, C₂₋₆fluoroalkenyl, C₂₋₆alkynyl and C₂₋₆fluoroalkynyl; or R¹⁰ and R¹¹ together form a 3 to 6-membered cycloalkyl ring, a 3 to 6-membered fluorocycloalkyl ring, or a 4 to 6-membered oxygen containing heterocyclic ring;
R¹² and R¹³ are independently selected from the group consisting of: H, C₁₋₆alkyl, fluoro, C₁₋₆fluoroalkyl, C₃₋₆cycloalkyl, C₃₋₆fluorocycloalkyl, C₂₋₆alkenyl, C₂₋₆fluoroalkenyl, C₂₋₆alkynyl and C₂₋₆fluoroalkynyl; or R¹² and R¹³ together form a 3 to 6-membered cycloalkyl ring, a 3 to 6-membered fluorocycloalkyl ring, or a 4 to 6-membered oxygen containing heterocyclic ring;
R¹⁵ and R¹⁶ are independently selected from the group consisting of: H, C₁₋₆alkyl, C₁₋₆fluoroalkyl, C₃₋₆cycloalkyl, C₃₋₆fluorocycloalkyl, C₂₋₆alkenyl, C₂₋₆fluoroalkenyl, C₂₋₆alkynyl and C₂₋₆fluoroalkynyl; or R¹⁵ and R¹⁶ together form a 3 to 6-membered cycloalkyl ring, a 3 to 6-membered fluorocycloalkyl ring, or a 4 to 6-membered oxygen containing heterocyclic ring;
or wherein:
one of R⁷ or R^{7′} and one of R⁸ or R⁹ together form a 5 or 6-membered cycloalkyl ring, a 5 or 6-membered fluorocycloalkyl ring, or a 5 or 6-membered oxygen containing heterocyclic ring;
one of R⁷ or R^{7′} and R⁶ together form a 3 to 6-membered cycloalkyl ring, a 3 to 6-membered fluorocycloalkyl ring, or a 4 to 6-membered oxygen containing heterocyclic ring;
R⁶ and one of R⁸ or R⁹ together form a 5 or 6-membered cycloalkyl ring, a 5 or 6-membered fluorocycloalkyl ring, or a 5 or 6-membered oxygen containing heterocyclic ring;
R⁶ and one of R¹⁰ or R¹¹ together form a 4 to 6-membered heterocyclyl ring or a 4 to 6-membered fluoroheterocyclyl ring;
one of R⁸ or R⁹ and one of R¹² or R¹³ together form a 4 to 7-membered heterocyclyl ring or a 4 to 7-membered fluoroheterocyclyl ring;
one of R⁸ or R⁹ and one of R¹⁵ or R¹⁶ together form a 5 to 7-membered heterocyclyl ring or a 5 to 7-membered fluoroheterocyclyl ring;
one of R¹⁰ or R¹¹ and one of R¹² or R¹³ together form a 3 to 6-membered cycloalkyl ring, a 3 to 6-membered fluorocycloalkyl ring, or a 5 or 6-membered oxygen containing heterocyclic ring;
one of R¹⁰ or R¹¹ and one of R¹⁵ or R¹⁶ together form a 5 or 6-membered cycloalkyl ring or a 5 or 6-membered fluorocycloalkyl ring;
one of R⁸ or R⁹ and one of R¹⁰ or R¹¹ together form a 5 to 7-membered heterocyclyl ring or a 5 to 7-membered fluoroheterocyclyl ring; and/or
one of R¹² or R¹³ and one of R¹⁵ or R¹⁶ together form a 3 to 6-membered cycloalkyl ring, a 3 to 6-membered fluorocycloalkyl ring, or a 5 or 6-membered oxygen containing heterocyclic ring.

2. A compound of formula (I) or a pharmaceutically acceptable salt or prodrug thereof: 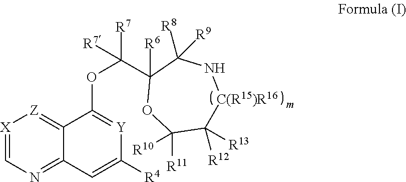
wherein:
Z is CR¹ or N;
Y is CH or N;
X is CR²; and
no more than one of X, Y or Z is N;
wherein:
R¹ is selected from the group consisting of: hydrogen, C₁₋₆alkyl, C₁₋₆fluoroalkyl, C₂₋₆alkenyl, C₂₋₆fluoroalkenyl, C₂₋₆alkynyl, C₂₋₆fluoroalkynyl, C₃₋₆cycloalkyl, halo, —O—C₁₋₆alkyl, —O—C₁₋₆fluoroalkyl, —O—C₂₋₆alkenyl, —O—C₂₋₆fluoroalkenyl, —O—C₂₋₆alkynyl, —O—C₂₋₆fluoroalkynyl and cyano;
R² is selected from the group consisting of: hydrogen, C₁₋₆alkyl, C₁₋₆fluoroalkyl, C₂₋₆alkenyl, C₂₋₆fluoroalkenyl, C₂₋₆alkynyl, C₂₋₆fluoroalkynyl, halo, —O—C₁₋₆alkyl, —O—C₁₋₆fluoroalkyl, —O—C₂₋₆alkenyl, —O—C₂₋₆fluoroalkenyl, —O—C₂₋₆alkynyl, —O—C₂₋₆fluoroalkynyl and cyano;
R⁴ is 5-membered cycloalkene or 5-membered heteroaryl, each of which is optionally fused to form a 5:6, or 5:5 aromatic or heteroaromatic bicycle; wherein each R⁴ is optionally substituted by one or more R⁵; wherein each R⁵ is independently selected from the group consisting of: —R¹⁴, —R¹⁴-cycloalkyl-R¹⁹, —R¹⁴-cyclofluoroalkyl-R¹⁹, —R¹⁴-heterocyclyl-R¹⁹, —R¹⁴-fluoroheterocyclyl-R¹⁹, —R¹⁴-heteroaryl-R¹⁹, —R¹⁴-aryl-R¹⁹, -cycloalkyl-R¹⁹, -cyclofluoroalkyl-R¹⁹, -heterocyclyl-R¹⁹, -fluoroheterocyclyl-R¹⁹, -heteroaryl-R¹⁹, -aryl-R¹⁹, —R¹⁴—O—R¹⁹, Cl, F, cyano, —OR¹⁹, —SR¹⁹, —SOR¹⁹, —SO₂R¹⁹, —N(R¹⁹)₂,—N(R¹⁹)COR¹⁹, —CON(R¹⁹)₂,—N(R¹⁹)CON(R¹⁹)₂,—N(R¹⁹)COOR¹⁹, —OCON(R¹⁹)₂, —N(R¹⁹)SO₂R¹⁹, —SO₂N(R¹⁹)₂, and ═O; wherein each R¹⁴ is independently selected from the group consisting of C₁₋₆alkyl, C₂₋₆alkenyl, C₂₋₆alkynyl, C₁₋₆fluoroalkyl, C₂₋₆fluoroalkenyl, C₂₋₆fluoroalkynyl and C₃₋₆cycloalkyl; wherein each R¹⁹ is independently selected from the group consisting of H, C₁₋₆alkyl, C₂₋₆alkenyl, C₂₋₆alkynyl, C₁₋₆fluoroalkyl, C₂₋₆fluoroalkenyl, C₂₋₆fluoroalkynyl and C₃₋₆cycloalkyl;
m is 0 or 1;
R⁶ is selected from the group consisting of: H, C₁₋₆alkyl, C₁₋₆fluoroalkyl, C₃₋₆cycloalkyl, C₃₋₆fluorocycloalkyl, C₂₋₆alkenyl, C₂₋₆fluoroalkenyl, C₂₋₆alkynyl and C₂₋₆fluoroalkynyl;
R⁷ and R^{7′} are independently selected from the group consisting of: H, fluoro, C₁₋₆alkyl, C₁₋₆fluoroalkyl, C₃₋₆cycloalkyl, C₃₋₆fluorocycloalkyl, C₂₋₆alkenyl, C₂₋₆fluoroalkenyl, C₂₋₆alkynyl and C₂₋₆fluoroalkynyl; or R⁷ and R^{7′} together form a 3 to 6-membered cycloalkyl ring, a 3 to 6-membered fluorocycloalkyl ring, or a 4 to 6-membered oxygen containing heterocyclic ring;
R⁸ and R⁹ are independently selected from the group consisting of: H, C₁₋₆alkyl, C₁₋₆fluoroalkyl, C₃₋₆cycloalkyl, C₃₋₆fluorocycloalkyl, C₂₋₆alkenyl, C₂₋₆fluoroalkenyl, C₂₋₆alkynyl and C₂₋₆fluoroalkynyl; or R⁸ and R⁹ together form a 3 to 6-membered cycloalkyl ring, a 3 to 6-membered fluorocycloalkyl ring, or a 4 to 6-membered oxygen containing heterocyclic ring;
R¹⁰ and R¹¹ are independently selected from the group consisting of: H, C₁₋₆alkyl, fluoro, C₁₋₆fluoroalkyl, C₃₋₆cycloalkyl, C₃₋₆fluorocycloalkyl, C₂₋₆alkenyl, C₂₋₆fluoroalkenyl, C₂₋₆alkynyl and C₂₋₆fluoroalkynyl; or R¹⁰ and R¹¹ together form a 3 to 6-membered cycloalkyl ring, a 3 to 6-membered fluorocycloalkyl ring, or a 4 to 6-membered oxygen containing heterocyclic ring;
R¹² and R¹³ are independently selected from the group consisting of: H, C₁₋₆alkyl, fluoro, C₁₋₆fluoroalkyl, C₃₋₆cycloalkyl, C₃₋₆fluorocycloalkyl, C₂₋₆alkenyl, C₂₋₆fluoroalkenyl, C₂₋₆alkynyl and C₂₋₆fluoroalkynyl; or R¹² and R¹³ together form a 3 to 6-membered cycloalkyl ring, a 3 to 6-membered fluorocycloalkyl ring, or a 4 to 6-membered oxygen containing heterocyclic ring;
R¹⁵ and R¹⁶ are independently selected from the group consisting of: H, C₁₋₆alkyl, C₁₋₆fluoroalkyl, C₃₋₆cycloalkyl, C₃₋₆fluorocycloalkyl, C₂₋₆alkenyl, C₂₋₆fluoroalkenyl, C₂₋₆alkynyl and C₂₋₆fluoroalkynyl; or R¹⁵ and R¹⁶ together form a 3 to 6-membered cycloalkyl ring, a 3 to 6-membered fluorocycloalkyl ring, or a 4 to 6-membered oxygen containing heterocyclic ring;
or wherein:
one of R⁷ or R^{7′} and one of R⁸ or R⁹ together form a 5 or 6-membered cycloalkyl ring, a 5 or 6-membered fluorocycloalkyl ring, or a 5 or 6-membered oxygen containing heterocyclic ring;
one of R⁷ or R^{7′} and R⁶ together form a 3 to 6-membered cycloalkyl ring, a 3 to 6-membered fluorocycloalkyl ring, or a 4 to 6-membered oxygen containing heterocyclic ring;
R⁶ and one of R⁸ or R⁹ together form a 5 or 6-membered cycloalkyl ring, a 5 or 6-membered fluorocycloalkyl ring, or a 5 or 6-membered oxygen containing heterocyclic ring;
R⁶ and one of R¹⁰ or R¹¹ together form a 4 to 6-membered heterocyclyl ring or a 4 to 6-membered fluoroheterocyclyl ring;
one of R⁸ or R⁹ and one of R¹² or R¹³ together form a 4 to 7-membered heterocyclyl ring or a 4 to 7-membered fluoroheterocyclyl ring;
one of R⁸ or R⁹ and one of R¹⁵ or R¹⁶ together form a 5 to 7-membered heterocyclyl ring or a 5 to 7-membered fluoroheterocyclyl ring;
one of R¹⁰ or R¹¹ and one of R¹² or R¹³ together form a 3 to 6-membered cycloalkyl ring, a 3 to 6-membered fluorocycloalkyl ring, or a 5 or 6-membered oxygen containing heterocyclic ring;
one of R¹⁰ or R¹¹ and one of R¹⁵ or R¹⁶ together form a 5 or 6-membered cycloalkyl ring or a 5 or 6-membered fluorocycloalkyl ring;
one of R⁸ or R⁹ and one of R¹⁰ or R¹¹ together form a 5 to 7-membered heterocyclyl ring or a 5 to 7-membered fluoroheterocyclyl ring; and/or
one of R¹² or R¹³ and one of R¹⁵ or R¹⁶ together form a 3 to 6-membered cycloalkyl ring, a 3 to 6-membered fluorocycloalkyl ring, or a 5 or 6-membered oxygen containing heterocyclic ring.

3. The compound of claim 1, wherein the compound is a compound of Formula (II):

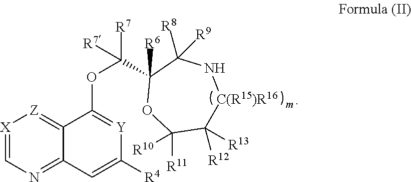

4. The compound of claim 1, wherein the compound is a compound of Formula (III):

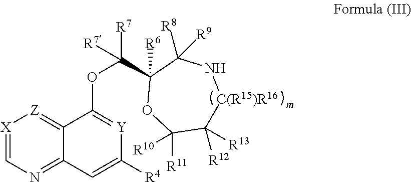

5. The compound of claim 1, wherein Y is N and Z is CR¹.

6. (canceled)

7. (canceled)

8. The compound of claim 1, wherein R⁴ is selected from the group consisting of: cyclopentenyl, pyrrolyl, 2,3-dihydro-pyrrolizinyl, pyrazolyl, 4,5,6,7-tetrahydropyrazolo[1,5-a]pyridinyl, thiophenyl, 1,2-oxazolyl, 1,3-thiazolyl, and 1,2-thiazolyl; wherein said R⁴ groups are optionally substituted by one or more R⁵; wherein each R⁵ is independently selected from the group consisting of: —R¹⁴, —R¹⁴-cycloalkyl-R¹⁹, —R¹⁴-cyclofluoroalkyl-R¹⁹, —R¹⁴-heterocyclyl-R¹⁹, —R¹⁴-fluoroheterocyclyl-R¹⁹, —R¹⁴-heteroaryl-R¹⁹, —R¹⁴-aryl-R¹⁹, -cycloalkyl-R¹⁹, -cyclofluoroalkyl-R¹⁹, -heterocyclyl-R¹⁹, -fluoroheterocyclyl-R¹⁹, -heteroaryl-R¹⁹, -aryl-R¹⁹, —R¹⁴—O—R¹⁹, Cl, F, cyano, —OR¹⁹, —SR¹⁹, —SOR¹⁹, —SO₂R¹⁹, —N(R¹⁹)₂, —N(R¹⁹)COR¹⁹, —CON(R¹⁹)₂,—N(R¹⁹)CON(R¹⁹)₂,—N(R¹⁹)COOR¹⁹, —OCON(R¹⁹)₂, —N(R¹⁹)SO₂R¹⁹, —SO₂N(R¹⁹)₂, and ═O; wherein each R¹⁴ is independently selected from the group consisting of C₁₋₆alkyl, C₂₋₆alkenyl, C₂₋₆alkynyl, C₁₋₆fluoroalkyl, C₂₋₆fluoroalkenyl, C₂₋₆fluoroalkynyl and C₃₋₆cycloalkyl; wherein each R¹⁹ is independently selected from the group consisting of H, C₁₋₆alkyl, C₂₋₆alkenyl, C₂₋₆alkynyl, C₁₋₆fluoroalkyl, C₂₋₆fluoroalkenyl, C₂₋₆fluoroalkynyl and C₃₋₆cycloalkyl.

9. The compound of claim 1, wherein R⁴ is selected from the group consisting of: 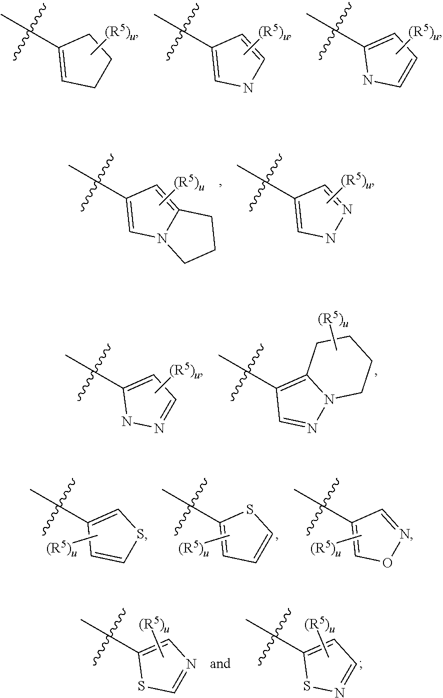
wherein u is an integer from 0 to the maximum number of substituent positions on said group;
wherein each R⁵ is independently selected from the group consisting of: —R¹⁴, —R¹⁴-cycloalkyl-R¹⁹, —R¹⁴-cyclofluoroalkyl-R¹⁹, —R¹⁴-heterocyclyl-R¹⁹, —R¹⁴-fluoroheterocyclyl-R¹⁹, —R¹⁴-heteroaryl-R¹⁹, —R¹⁴-aryl-R¹⁹, -cycloalkyl-R¹⁹, -cyclofluoroalkyl-R¹⁹, -heterocyclyl-R¹⁹, -fluoroheterocyclyl-R¹⁹, -heteroaryl-R¹⁹, -aryl-R¹⁹, —R¹⁴—O—R¹⁹, Cl, F, cyano, —OR¹⁹, —SR¹⁹, —SOR¹⁹, —SO₂R¹⁹, —N(R¹⁹)₂, —N(R¹⁹)COR¹⁹, —CON(R¹⁹)₂, —N(R¹⁹)CON(R¹⁹)₂, —N(R¹⁹)COOR¹⁹, —OCON(R¹⁹)₂, —N(R¹⁹)SO₂R¹⁹, —SO₂N(R¹⁹)₂, and ═O; wherein each R¹⁴ is independently selected from the group consisting of C₁₋₆alkyl, C₂₋₆alkenyl, C₂₋₆alkynyl, C₁₋₆fluoroalkyl, C₂₋₆fluoroalkenyl, C₂₋₆fluoroalkynyl and C₃₋₆cycloalkyl; wherein each R¹⁹ is independently selected from the group consisting of H, C₁₋₆alkyl, C₂₋₆alkenyl, C₂₋₆alkynyl, C₁₋₆fluoroalkyl, C₂₋₆fluoroalkenyl, C₂₋₆fluoroalkynyl and C₃₋₆cycloalkyl.

10. The compound of claim 8, wherein each R⁵ is independently selected from the group consisting of: —R¹⁴, —R¹⁴-cycloalkyl-R¹⁹, -R¹⁴cyclofluoroalkyl-R¹⁹, —R¹⁴-heterocyclyl-R¹⁹, —R¹⁴-fluoroheterocyclyl-R¹⁹, —R¹⁴-heteroaryl-R¹⁹, —R¹⁴-aryl-R¹⁹, -cycloalkyl-R¹⁹, -cyclofluoroalkyl-R¹⁹, -heterocyclyl-R¹⁹, -fluoroheterocyclyl-R¹⁹, -heteroaryl-R¹⁹, -aryl-R¹⁹, —R¹⁴—O—R¹⁹, Cl, F, cyano, —OR¹⁹, —SR¹⁹ and ═O; wherein each R¹⁴ is independently selected from the group consisting of C₁₋₆alkyl, C₂₋₆alkenyl, C₂₋₆alkynyl, C₁₋₆fluoroalkyl, C₂₋₆fluoroalkenyl and C₂₋₆fluoroalkynyl; wherein each R¹⁹ is independently selected from the group consisting of H, C₁₋₆alkyl, C₂₋₆alkenyl, C₂₋₆alkynyl, C₁₋₆fluoroalkyl, C₂₋₆fluoroalkenyl and C₂₋₆fluoroalkynyl.

11. The compound of claim 1, wherein R⁸ and R⁹ are independently selected from the group consisting of: hydrogen, C₁₋₆alkyl and C₁₋₆fluoroalkyl.

12. The compound of claim 1, wherein R¹⁰ and R¹¹ are independently selected from the group consisting of: H, C₁₋₆alkyl, fluoro and C₁₋₆fluoroalkyl; or R¹⁰ and R¹¹ together form a 3 to 6-membered cycloalkyl ring, a 3 to 6-membered fluorocycloalkyl ring, or a 4 to 6-membered oxygen containing heterocyclic ring.

13. The compound of claim 1, wherein R¹² and R¹³ are independently selected from the group consisting of: H, C₁₋₆alkyl, fluoro and C₁₋₆fluoroalkyl.

14. The compound of claim 1, wherein m is 0.

15. The compound of claim 1, wherein in the compound of Formula (I), 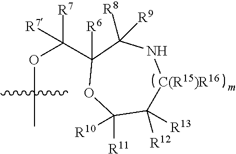
is selected from the group consisting of:

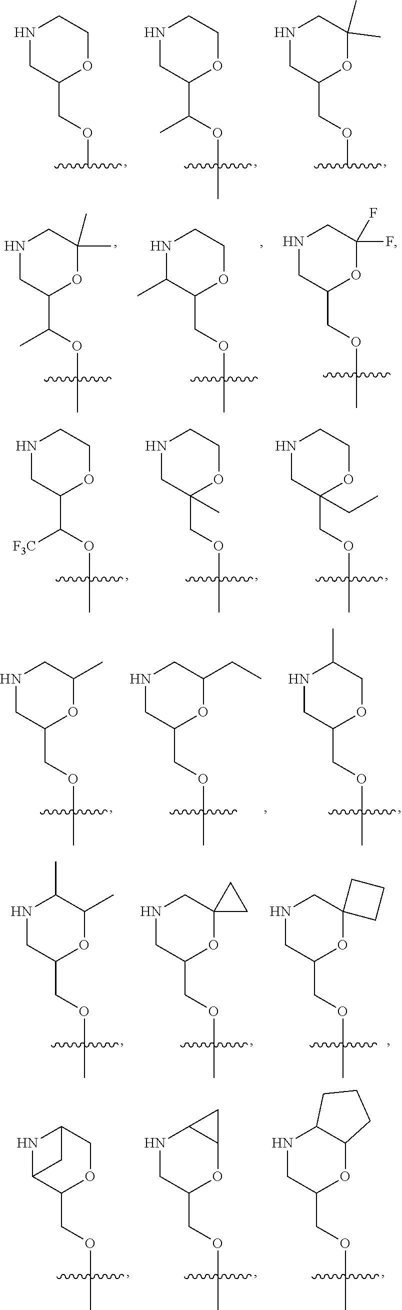

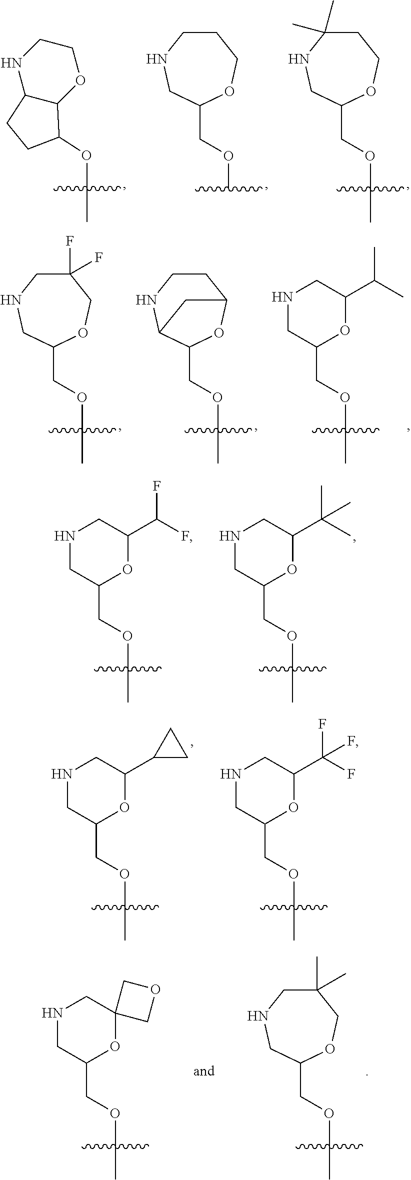

16. The compound of claim 1, wherein the compound of Formula (I) is selected from the group consisting of:

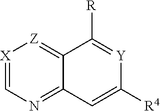

| No | X | Y | Z | R | R⁴ |
|---|---|---|---|---|---|
| 2.44 | CH | N | CH | 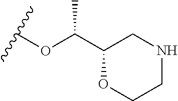 [SMILES: *O[C@H](C)[C@@H]1CNCCO1] | 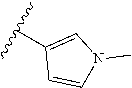 [SMILES: *c1ccn(C)c1] |
| 2.51 | CH | N | CH | 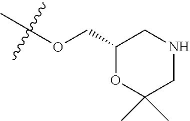 [SMILES: *OC[C@@H]1CNCC(C)(C)O1] | 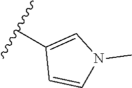 [SMILES: *c1ccn(C)c1] |
| 2.20 | CH | N | CH | 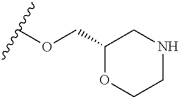 [SMILES: *OC[C@@H]1CNCCO1] | 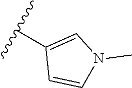 [SMILES: *c1ccn(C)c1] |
| 3.10 | CH | CH | CH | 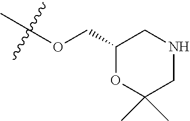 [SMILES: *OC[C@@H]1CNCC(C)(C)O1] | 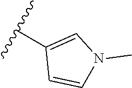 [SMILES: *c1ccn(C)c1] |
| 6.28 | CH | N | CH | 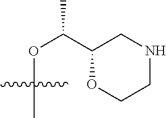 [SMILES: *O[C@H](C)[C@@H]1CNCCO1] | 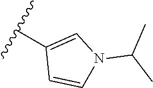 [SMILES: *c1ccn(C(C)C)c1] |
| 6.26 | CH | N | CH | 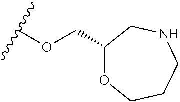 [SMILES: *OC[C@@H]1CNCCCO1] | 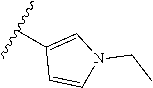 [SMILES: *c1ccn(CC)c1] |
| 3.1 | CH | CH | CH | 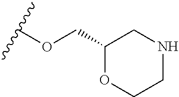 [SMILES: *OC[C@@H]1CNCCO1] | 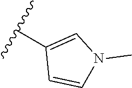 [SMILES: *c1ccn(C)c1] |
| 2.42 | CH | N | CH | 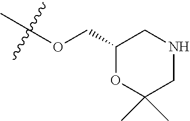 [SMILES: *OC[C@@H]1CNCC(C)(C)O1] | 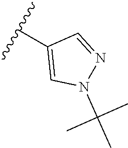 [SMILES: *c1cnn(C(C)(C)C)c1] |
| 6.9 | CH | N | CH | 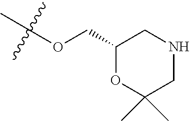 [SMILES: *OC[C@@H]1CNCC(C)(C)O1] | 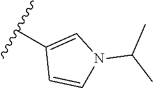 [SMILES: *c1ccn(C(C)C)c1] |
| 6.19 | CH | N | CH | 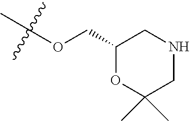 [SMILES: *OC[C@@H]1CNCC(C)(C)O1] | 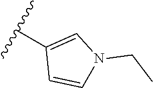 [SMILES: *c1ccn(CC)c1] |
| 2.67 | CH | N | CH | 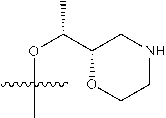 [SMILES: *O[C@H](C)[C@@H]1CNCCO1] | 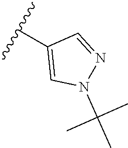 [SMILES: *c1cnn(C(C)(C)C)c1] |
| 6.2 | CH | N | CH | 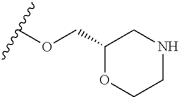 [SMILES: *OC[C@@H]1CNCCO1] | 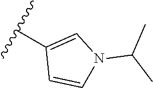 [SMILES: *c1ccn(C(C)C)c1] |
| 14.6 | CH | N | C-CH₃ | 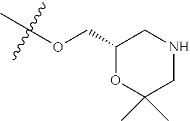 [SMILES: *OC[C@@H]1CNCC(C)(C)O1] | 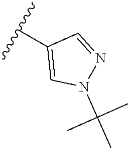 [SMILES: *c1cnn(C(C)(C)C)c1] |
| 3.6 | CH | CH | CH | 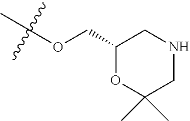 [SMILES: *OC[C@@H]1CNCC(C)(C)O1] | 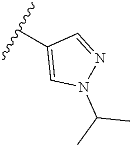 [SMILES: *c1cnn(C(C)C)c1] |
| 7.11 | CH | CH | CH | 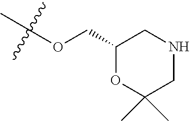 [SMILES: *OC[C@@H]1CNCC(C)(C)O1] | 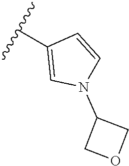 [SMILES: *c1ccn(C2COC2)c1] |
| 2.55 | CH | N | CH | 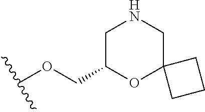 [SMILES: *OC[C@@H]1CNCC2(CCC2)O1] | 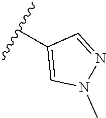 [SMILES: *c1cnn(C)c1] |
| 2.66 | CH | N | CH | 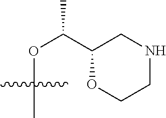 [SMILES: *O[C@H](C)[C@@H]1CNCCO1] | 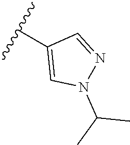 [SMILES: *c1cnn(C(C)C)c1] |
| 12.8 | CH | CH | N | 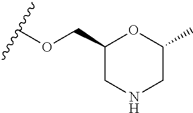 [SMILES: *OC[C@@H]1CNC[C@@H](C)O1] | 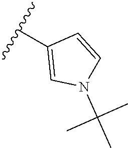 [SMILES: *c1ccn(C(C)(C)C)c1] |
| 4 | CH | CH | CH | 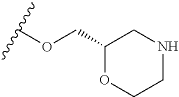 [SMILES: *OC[C@@H]1CNCCO1] | 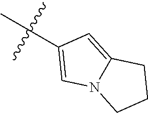 [SMILES: *c1cc2n(c1)CCC2] |
| 14.2 | CH | CH | CH | 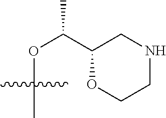 [SMILES: *O[C@H](C)[C@@H]1CNCCO1] | 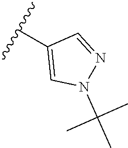 [SMILES: *c1cnn(C(C)(C)C)c1] |
| 1 | CH | CH | CH | 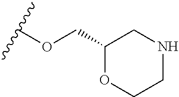 [SMILES: *OC[C@@H]1CNCCO1] | 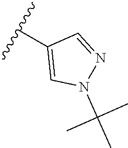 [SMILES: *c1cnn(C(C)(C)C)c1] |
| 6.22 | CH | N | CH | 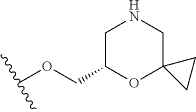 [SMILES: *OC[C@@H]1CNCC2(CC2)O1] | 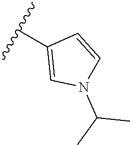 [SMILES: *c1ccn(C(C)C)c1] |
| 2.99 | CH | N | CH | 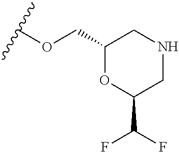 [SMILES: *OC[C@@H]1CNC[C@@H](C(F)F)O1] | 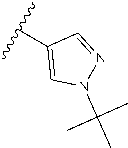 [SMILES: *c1cnn(C(C)(C)C)c1] |

17. A pharmaceutical composition comprising an effective amount of the compound of claim 1, or a pharmaceutically acceptable salt or prodrug thereof; wherein the pharmaceutical composition further comprises a pharmaceutically acceptable carrier, diluent and/or excipient.

18. (canceled)

19. A method of treating or preventing a disease, disorder or condition associated with spleen tyrosine kinase activity in a subject, the method comprising administering to the subject an effective amount of the compound of claim 1 or a pharmaceutically acceptable salt or prodrug thereof.

20. The method of claim 19, wherein the disease, disorder or condition associated with spleen tyrosine kinase activity may be selected from one or more of the group consisting of: glioblastoma, cancer, osteoporosis, rheumatoid arthritis, liver disease, fibrosis, periodontal diseases, diabetes, inflammation, Graves' disease, lung diseases or disorders, kidney disease, epidermolysis bullosa acquisita, Wiskott-Aldrich syndrome, agammaglobulinemia, Nasu-Hakola disease, allergy, microbial infection, fungal infection, autoimmune hypersensitivity disease, bleeding disorders, thrombocytopenia, bone or skeletal disorders, nail disease, chronic mucocutaneous candidiasis, a neurological disease or disorder, a neuroinflammatory disease, stroke, traumatic brain injury, and subarachnoid haemorrhage.

21. The method of claim 19, wherein the disease, disorder or condition associated with spleen tyrosine kinase activity is in the Central Nervous System.
